(19) United States
(12) Patent Application Publication
(10) Pub. No.: US 2018/0212158 A1
(43) Pub. Date: Jul. 26, 2018

(54) ORGANIC LIGHT-EMITTING DIODE MATERIALS

(71) Applicant: President and Fellows of Harvard College, Cambridge, MA (US)

(72) Inventors: Alan Aspuru-Guzik, Cambridge, MA (US); Rafael Gomez-Bombarelli, Cambridge, MA (US); Timothy D. Hirzel, Quincy, MA (US); Jorge Aguilera-Iparraguirre, Roslindale, MA (US)

(21) Appl. No.: 15/744,571

(22) PCT Filed: Jul. 13, 2016
(86) PCT No.: PCT/US16/42052

§ 371 Date: Jan. 12, 2018

Related U.S. Application Data
(60) Provisional application No. 62/277,316, filed on Jan. 11, 2016.
(60) Provisional application No. 62/239,556, filed on Oct. 9, 2015.
(60) Provisional application No. 62/208,190, filed on Aug. 21, 2015.
(60) Provisional application No. 62/191,766, filed on Jul. 13, 2015.

(51) Int. Cl. H01L 51/00; C07D 471/04; C09K 11/06; C07D 209/88; C07D 519/00; C07D 417/04; C07D 403/14; C07D 487/04

(52) U.S. Cl. CPC H01L 51/0072; C07D 471/04; C09K 11/06; H01L 51/0067; H01L 51/0061; H01L 51/5012; C07D 519/00; C07D 417/04; C07D 403/14; C07D 487/04; C09K 2211/1018; C07D 209/88

(57) ABSTRACT

Described herin are molecules for use in organic light emitting diodes. Example molecules comprise at least one acceptor moiety A, at least one donor moiety D, and optionally one or more bridge moieties B. Each moiety A is covalently attached to either the moiety B or the moeity D, each moiety D is covalently attached to either the moeity B or the moeity A, and each B is covalently attached to at least one moiety A and at least one moiety D. Values and preferred values of moieties A, D and B are defined herein.

### RELATED APPLICATIONS

This application claims the benefit of U.S. Provisional Application No. 62/191766, filed on Jul. 13, 2015; U.S. Provisional Application No. 62/208190, filed on Aug. 21, 2015; U.S. Provisional Application No. 62/239556, filed on Oct. 9, 2015; U.S. Provisional Application No. 62/277316, filed on Jan. 11, 2016. The entire teachings of each application above are incorporated herein by reference.

### BACKGROUND OF THE INVENTION

An organic light emitting diode (OLED) is a light-emitting diode (LED) in which a film of organic compounds is placed between two conductors and emits light in response to excitation, such as an electric current. OLEDs are useful in displays such as television screen, computer monitors, mobile phones, and tablets. A problem inherent in OLED displays is the limited lifetime of the organic materials. OLEDs which emit blue light, in particular, degrade at a significantly increased rate as compared to green or red OLEDs.

OLED materials rely on the radiative decay of molecular excited states (excitons) generated by recombination of electrons and holes in a host transport material. The nature of excitation results in interactions between electrons and holes that split the excited states into bright singlets (with a total spin of 0) and dark triplets (with a total spin of 1). Since the recombination of electrons and holes affords a statistical mixture of four spin states (one singlet and three triplet sublevels), conventional OLEDs have a maximum theoretical efficiency of 25%.

To date, OLED material design has focused on harvesting the remaining energy from the normally dark triplets into an emissive state. Recent work to create efficient phosphors, which emit light from the normally dark triplet state, have resulted in green and red OLEDs. Other colors, such as blue, however, require higher energy excited states which enhance the degradation process of the OLED.

The fundamental limiting factor to the triplet-singlet transition rate is a value of the parameter |H_{fi}/Δ|², where H_{fi}, is the coupling energy due to hyperfine or spin-orbit interactions, and Δ is the energetic splitting between singlet and triplet states. Traditional phosphorescent OLEDs rely on the mixing of singlet and triplet states due to spin-orbital (SO) interaction, increasing H_{fi} and affording a lowest emissive state shared between a heavy metal atom and an organic ligand. This results in energy harvesting from all higher singlet and triplet states, followed by phosphorescence (relatively short-lived emission from the excited triplet). The shortened triplet lifetime reduces triplet exciton annihilation by charges and other excitons. Recent work by others suggests that the limit to the performance of phosphorescent materials has been reached.

### SUMMARY OF THE INVENTION

Thus, a need exists for OLEDs which can reach higher excitation states without rapid degradation. It has now been discovered that thermally activated delayed fluorescence (TADF), which relies on minimization of Δ as opposed to maximization of H_{fi}, can transfer population between singlet levels and triplet sublevels in a relevant timescale, such as, for example, 110 μs. The compounds described herein are capable of fluorescing or phosphorescing at higher energy excitation states than compounds previously described.

Accordingly, in one embodiment, the present invention is a molecule represented by one of structural formulas (I), (II), (IIIA)-(IIIE), (IIIC), (IV), (VA)-(VL), (VI), (VIIA)-(VIIE), or (VIIIA)-(VIIIF).

In another embodiment, the present invention is a molecule represented by one of the structural formulas in Tables M, N, O, Q, B, or R.

In another embodiment, the present invention is an organic light-emitting device comprising a first electrode, a second electrode, and an organic layer between the first electrode and the second electrode. The organic layer comprises at least one light-emitting molecule selected from the compounds disclosed herein.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1-21 represent Table 1 which lists example embodiments of the present invention.

FIGS. 22 to 40 represent Table 2 which lists example embodiments of the present invention.

FIGS. 41 to 48 represent Table 3 which lists example embodiments of the present invention.

FIGS. 49 to 57 represent Table 4 which lists example embodiments of the present invention.

FIGS. 58 to 72 represent Table 5 which lists example embodiments of the present invention.

FIGS. 73 to 89 represent Table 6 which lists example embodiments of the present invention.

FIGS. 90 to 91 represent Table 7 which lists example embodiments of the present invention.

FIGS. 92 to 93 represent Table 8 which lists example embodiments of the present invention.

FIGS. 94 to 98 represent Table 9 which lists example embodiments of the present invention.

FIGS. 99-104 represent Table 10, which lists example embodiments of the present invention.

FIGS. 105-107 represent Table 11, which lists example structures.

FIGS. 108-120 represent Table 12, which lists example embodiments of the present invention.

### DETAILED DESCRIPTION OF THE INVENTION

A description of example embodiments of the invention follows.

### Glossary

The term “alkyl,” as used herein, refers to a saturated aliphatic branched or straight-chain monovalent hydrocarbon radical having the specified number of carbon atoms. Thus, “C₁-C₆ alkyl” means a radical having from 1-6 carbon atoms in a linear or branched arrangement. Examples of “C₁-C₆ alkyl” include, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl. An alkyl can be optionally substituted with halogen, —OH, C₁-C₆ alkyl, C₁-C₆ alkoxy, —NO₂, —CN, and —N(R¹)(R²) wherein R¹ and R² are each independently selected from —H and C₁-C₃ alkyl.

The term “alkenyl,” as used herein, refers to a straight-chain or branched alkyl group having one or more carbon-carbon double bonds. Thus, “C₂-C₆ alkenyl” means a radical having 2-6 carbon atoms in a linear or branched arrangement having one or more double bonds. Examples of “C₂-C₆ alkenyl” include ethenyl, propenyl, butenyl, pentenyl, hexenyl, butadienyl, pentadienyl, and hexadienyl. An alkenyl can be optionally substituted with the substituents listed above with respect to alkyl.

The term “alkynyl,” as used herein, refers to a straight-chain or branched alkyl group having one or more carbon-carbon triple bonds. Thus, “C₂-C₆ alkynyl” means a radical having 2-6 carbon atoms in a linear or branched arrangement having one or more triple bonds. Examples of C₂-C₆ “alkynyl” include ethynyl, propynyl, butynyl, pentynyl, and hexynyl. An alkynyl can be optionally substituted with the substituents listed above with respect to alkyl.

The term “cycloalkyl,” as used herein, refers to a saturated monocyclic or fused polycyclic ring system containing from 3-12 carbon ring atoms. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. A cycloalkyl can be optionally substituted with the substituents listed above with respect to alkyl.

The term “amino,” as used herein, means an “—NH₂,” an “NHRₚ,” or an “NRₚR_{q},” group, wherein Rₚ and R_{q} can be alkyl, alkenyl, alkynyl, alkoxy, cycloalkyl, aryl, and heteroaryl. Amino may be primary (NH₂), secondary (NHRₚ) or tertiary (NRₚR_{q}).

The term “alkylamino,” as used herein, refers to an “NHRₚ,” or an “NRₚR_{q}” group, wherein Rₚ and R_{q} can be alkyl, alkenyl, alkynyl, alkoxy, or cycloalkyl. The term “dialkylamino,” as used herein, refers to an “NRₚR_{q}” group, wherein Rₚ and R_{q} can be alkyl, alkenyl, alkynyl, alkoxy, or cycloalkyl.

The term “alkoxy”, as used herein, refers to an “alkyl-O” group, wherein alkyl is defined above. Examples of alkoxy group include methoxy or ethoxy groups. The “alkyl” portion of alkoxy can be optionally substituted as described above with respect to alkyl.

The term “aryl,” as used herein, refers to an aromatic monocyclic or polycyclic ring system consisting of carbon atoms. Thus, “C₆-C₁₈ aryl” is a monocylic or polycyclic ring system containing from 6 to 18 carbon atoms. Examples of aryl groups include phenyl, indenyl, naphthyl, azulenyl, heptalenyl, biphenyl, indacenyl, acenaphthylenyl, fluorenyl, phenalenyl, phenanthrenyl, anthracenyl, cyclopentacyclooctenyl or benzocyclooctenyl. An aryl can be optionally substituted with halogen, —OH, C₁-C₆ alkyl, C₂-C₆ alkenyl, C₂-C₆ alkynyl, C₁-C₆ haloalkyl, C₁-C₆ alkoxy, C₆-C₁₈ aryl, C₆-C₁₈ haloaryl, (5-20 atom) heteroaryl, —C(O)C₁-C₃ haloalkyl, —S(O)₂—, —NO₂, —CN, and oxo.

The terms “halogen,” or “halo,” as used herein, refer to fluorine, chlorine, bromine, or iodine.

The term “heteroaryl,” as used herein, refers a monocyclic or fused polycyclic aromatic ring containing one or more heteroatoms, such as oxygen, nitrogen, or sulfur. For example, a heteroaryl can be a “5-20 atom heteroaryl,” which means a 5 to 20 membered monocyclic or fused polycyclic aromatic ring containing at least one heteroatom. Examples of heteroaryl groups include pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl. A heteroaryl can be optionally substituted with the same substituents listed above with respect to aryl.

In other embodiments, a “5-20 member heteroaryl” refers to a fused polycyclic ring system wherein aromatic rings are fused to a heterocycle. Examples of these heteroaryls include:

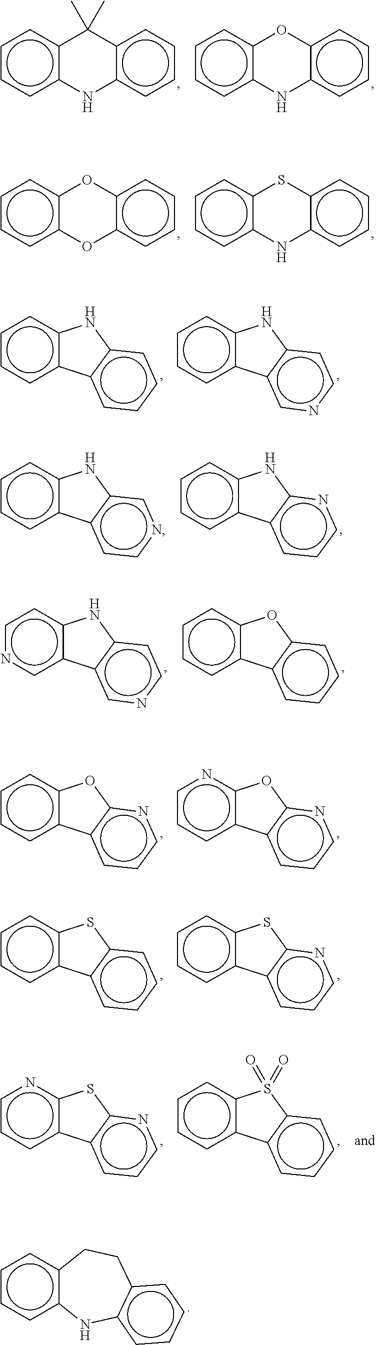

The term “haloalkyl,” as used herein, includes an alkyl substituted with one or more of F, Cl, Br, or I, wherein alkyl is defined above. The “alkyl” portion of haloalkyl can be optionally substituted as described above with respect to alkyl.

The term “haloaryl,” as used herein, includes an aryl substituted with one or more of F, Cl, Br, or I, wherein aryl is defined above. The “aryl” portion of haloaryl can be optionally substituted as described above with respect to aryl.

The term “oxo,” as used herein, refers to ═O.

The term “nitro,” as used herein, refers to —NO₂.

The term “symmetrical molecule,” as used herein, refers to molecules that are group symmetric or synthetic symmetric. The term “group symmetric,” as used herein, refers to molecules that have symmetry according to the group theory of molecular symmetry. The term “synthetic symmetric,” as used herein, refers to molecules that are selected such that no regioselective synthetic strategy is required.

The term “donor,” as used herein, refers to a molecular fragment that can be used in organic light emitting diodes and is likely to donate electrons from its highest occupied molecular orbital to an acceptor upon excitation. In an example embodiment, donors have an ionization potential greater than or equal to −6.5 eV.

The term “acceptor,” as used herein, refers to a molecular fragment that can be used in organic light emitting diodes and is likely to accept electrons into its lowest unoccupied molecular orbital from a donor that has been subject to excitation. In an example embodiment, acceptors have an electron affinity less than or equal to −0.5 eV.

The term “bridge,” as used herein, refers to π-conjugated molecular fragment that can be included in a molecule which is covalently linked between acceptor and donor moieties. The bridge can, for example, be further conjugated to the acceptor moiety, the donor moiety, or both. Without being bound to any particular theory, it is believed that the bridge moiety can sterically restrict the acceptor and donor moieties into a specific configuration, thereby preventing the overlap between the conjugated π system of donor and acceptor moieties. Examples of suitable bridge moieties include phenyl, ethenyl, and ethynyl.

The term “multivalent,” as used herein, refers to a molecular fragment that is connected to at least two other molecular fragments. For example, a bridge moiety, is multivalent.

“ [symbol] ” as used herein, refers to a point of attachment between two atoms.

### Principles of OLED

OLEDs are typically composed of a layer of organic materials or compounds between two electrodes, an anode and a cathode. The organic molecules are electrically conductive as a result of delocalization of π C electronics caused by conjugation over part or all of the molecule. When voltage is applied, electrons from the highest occupied molecular orbital (HOMO) present at the anode flow into the lowest unoccupied molecular orbital (LUMO) of the organic molecules present at the cathode. Removal of electrons from the HOMO is also referred to as inserting electron holes into the HOMO. Electrostatic forces bring the electrons and the holes towards each other until they recombine and form an exciton (which is the bound state of the electron and the hole). As the excited state decays and the energy levels of the electrons relax, radiation is emitted having a frequency in the visible spectrum. The frequency of this radiation depends on the band gap of the material, which is the difference in energy between the HOMO and the LUMO.

As electrons and holes are fermions with half integer spin, an exciton may either be in a singlet state or a triplet state depending on how the spins of the electron and hole have been combined. Statistically, three triplet excitons will be formed for each singlet exciton. Decay from triplet states is spin forbidden, which results in increases in the timescale of the transition and limits the internal efficiency of fluorescent devices. Phosphorescent organic light-emitting diodes make use of spin-orbit interactions to facilitate intersystem crossing between singlet and triplet states, thus obtaining emission from both singlet and triplet states and improving the internal efficiency.

The prototypical phosphorescent material is iridium tris(2-phenylpyridine) (Ir(ppy)₃) in which the excited state is a charge transfer from the Ir atom to the organic ligand. Such approaches have reduced the triplet lifetime to about 1 μs, several orders of magnitude slower than the radiative lifetimes of fully-allowed transitions such as fluorescence. Ir-based phosphors have proven to be acceptable for many display applications, but losses due to large triplet densities still prevent the application of OLEDs to solid-state lighting at higher brightness.

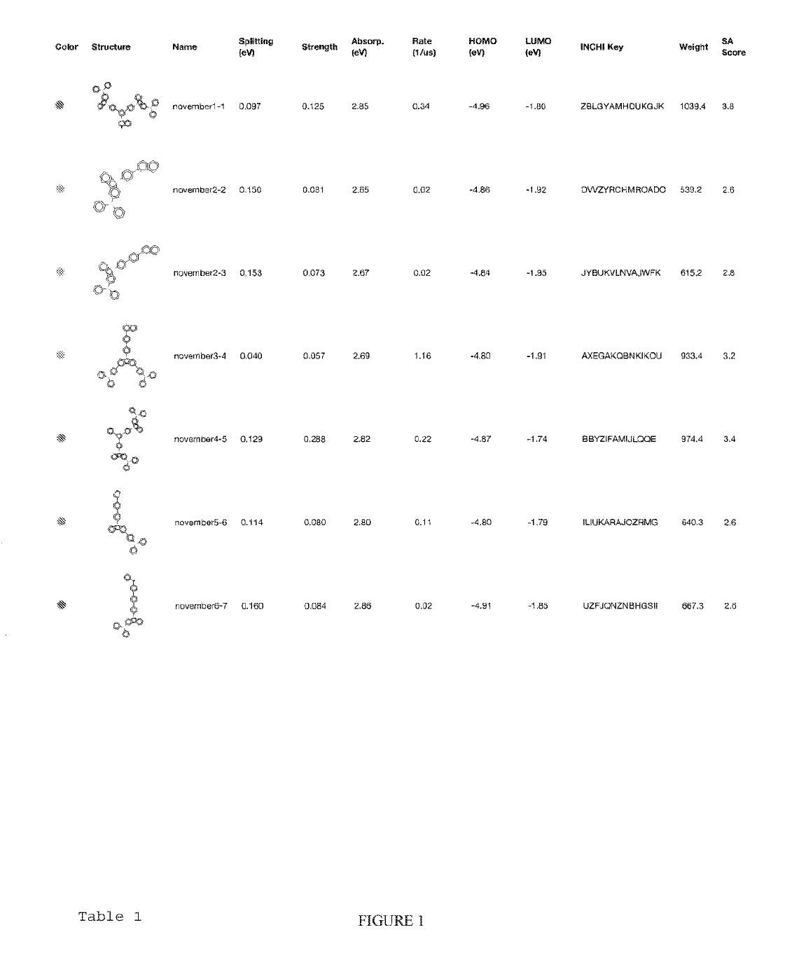

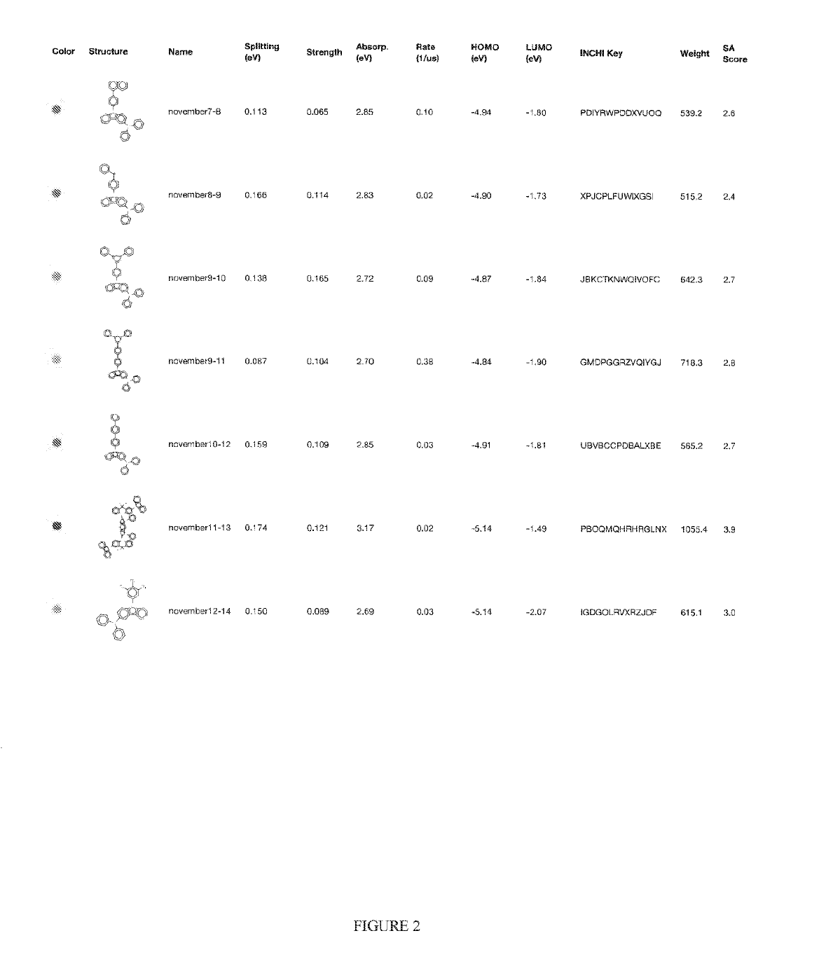

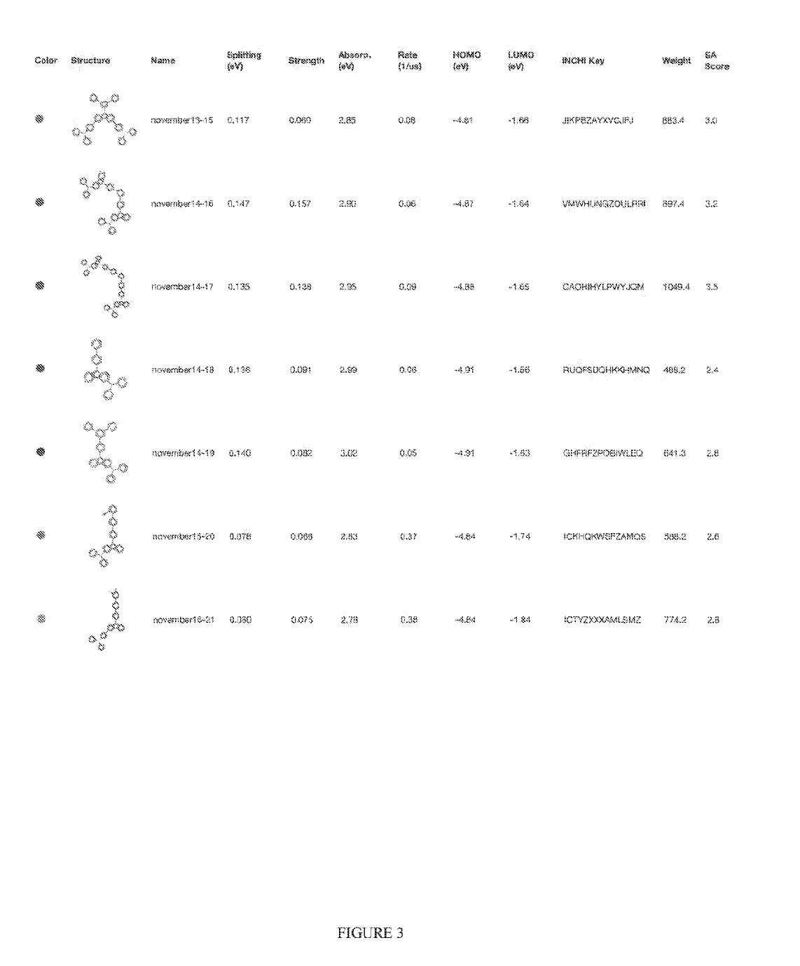

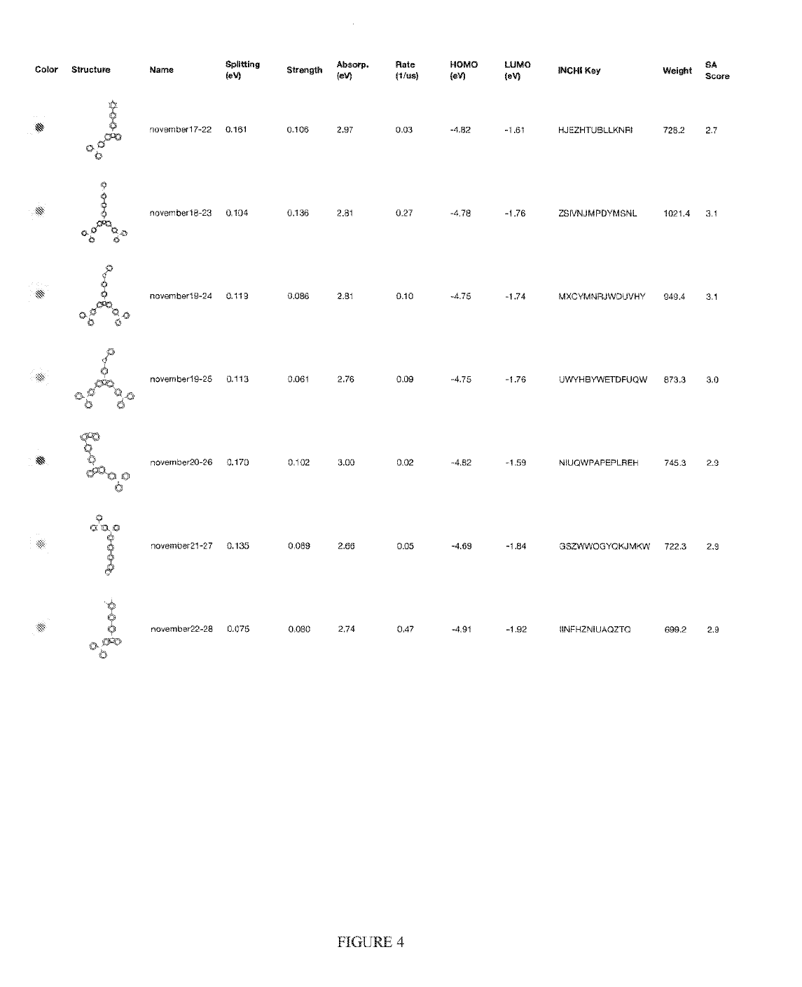

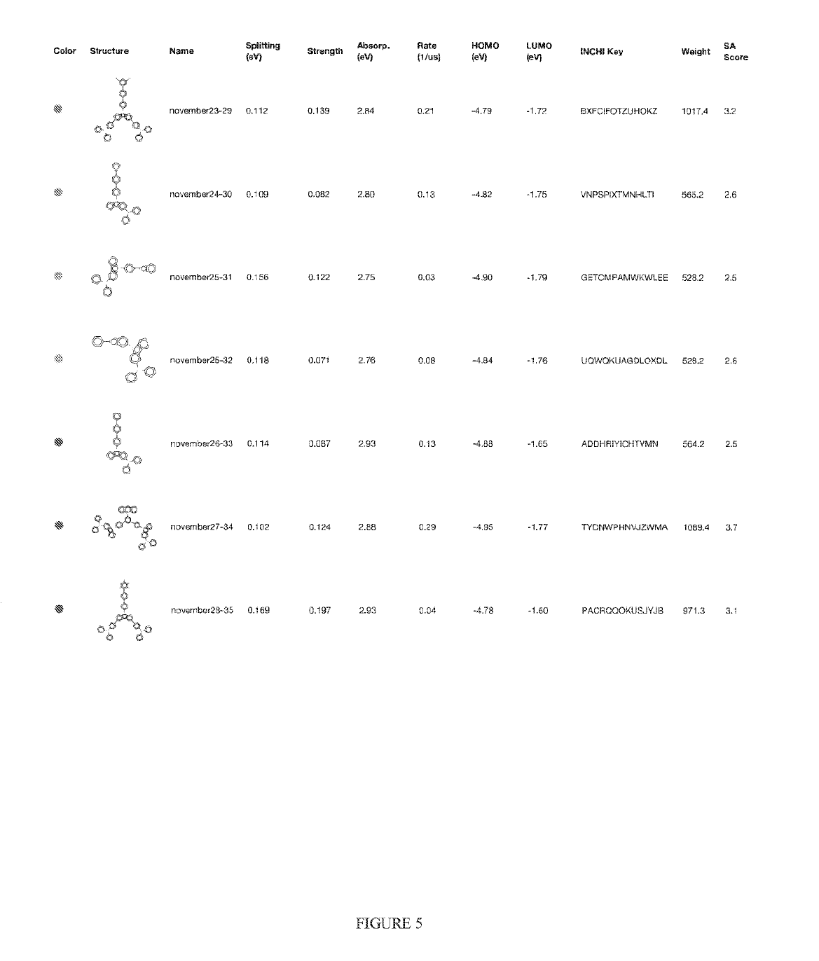

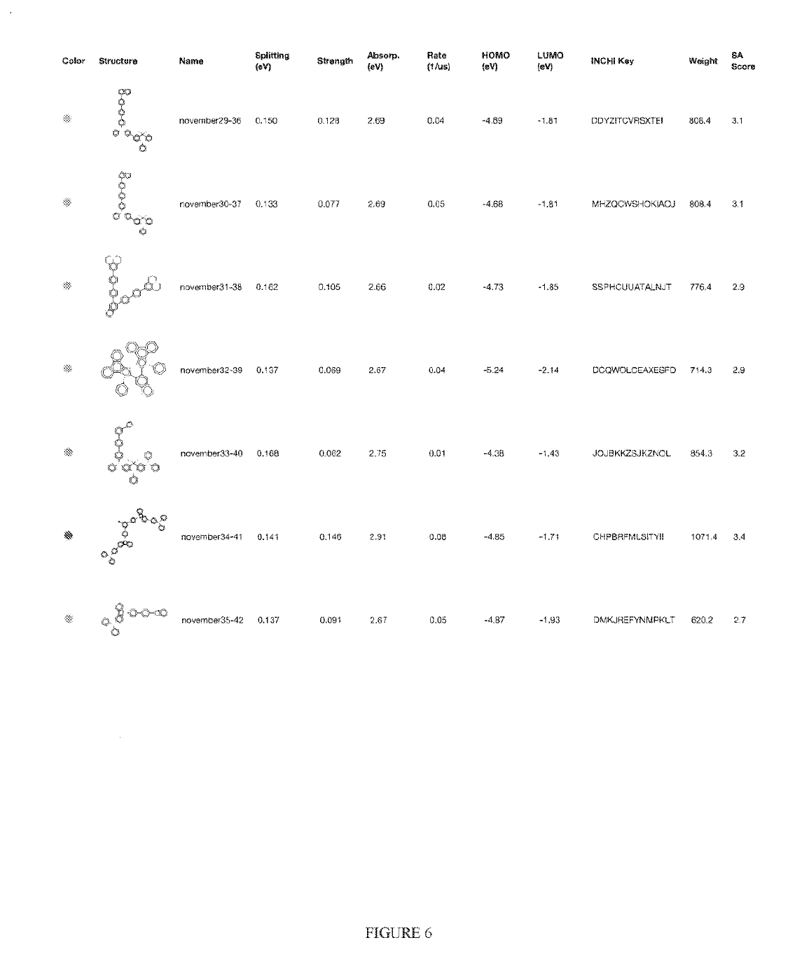

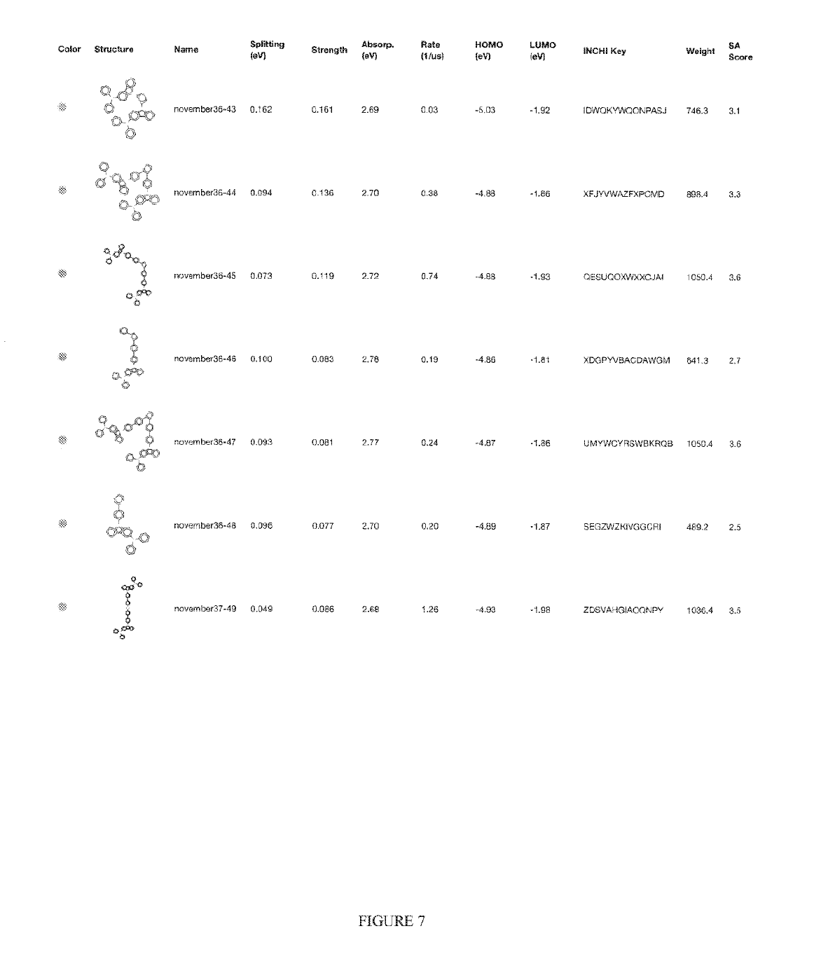

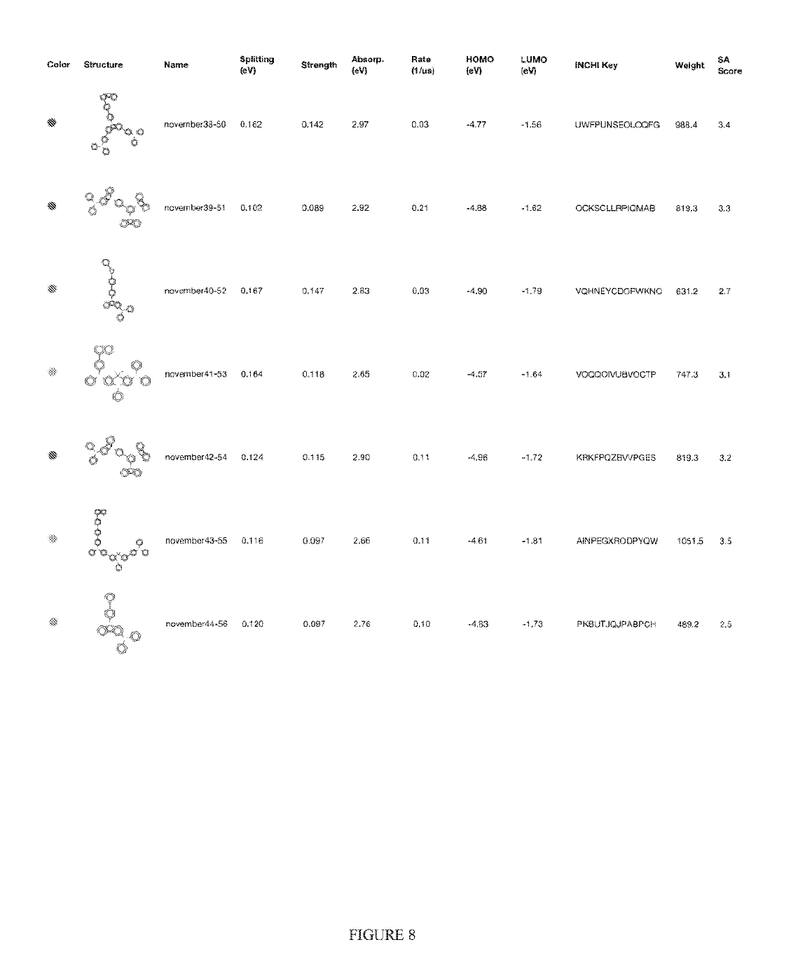

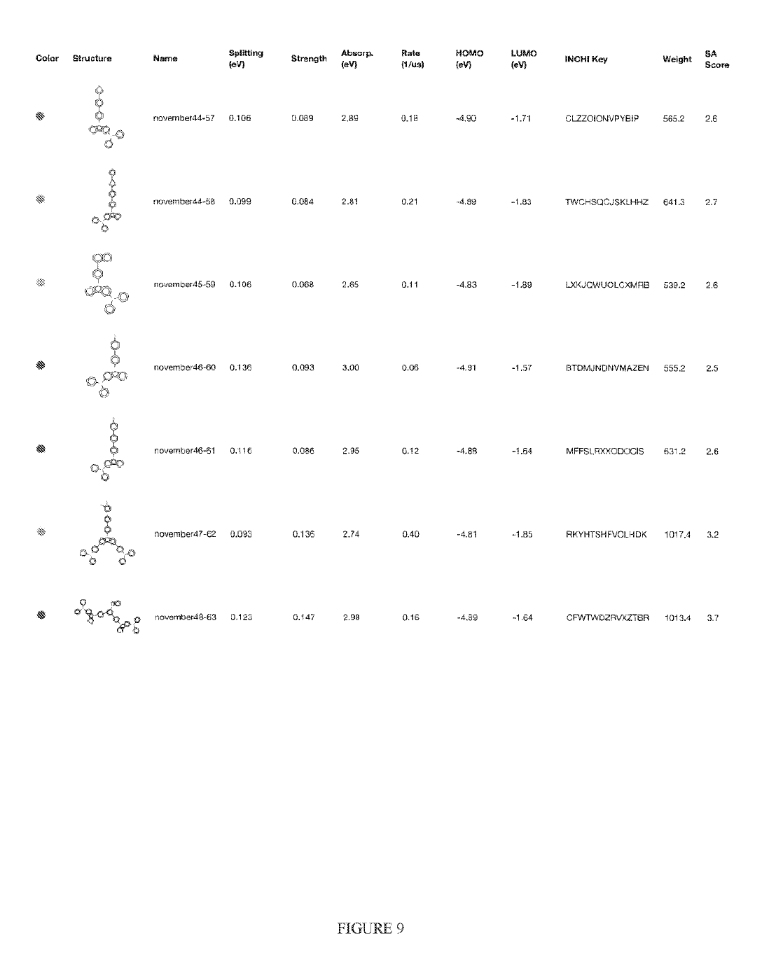

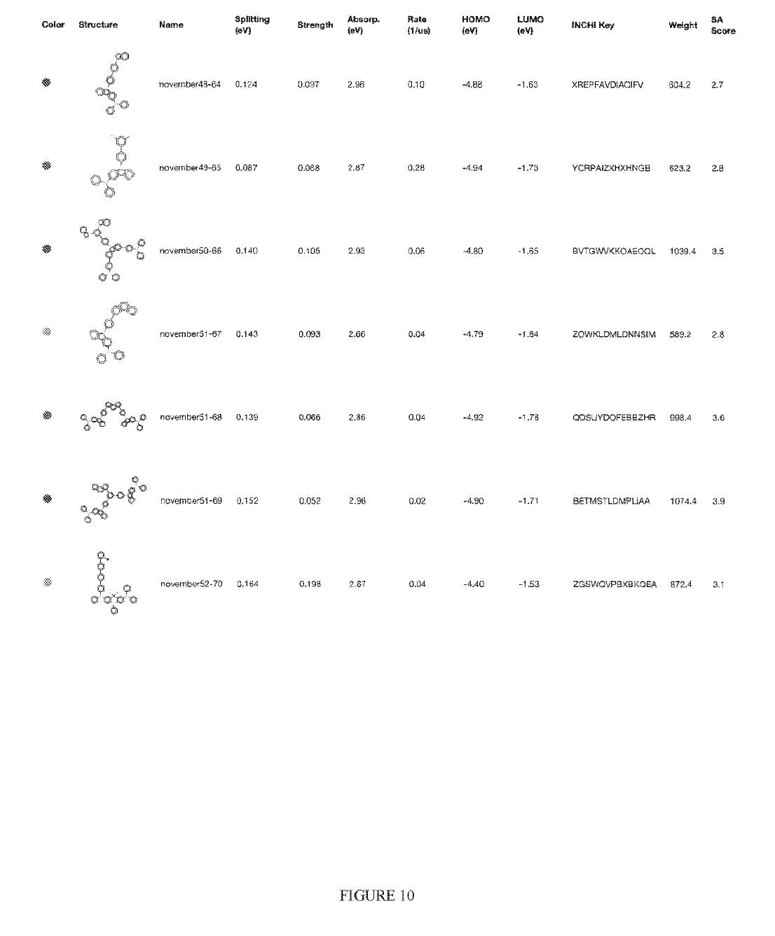

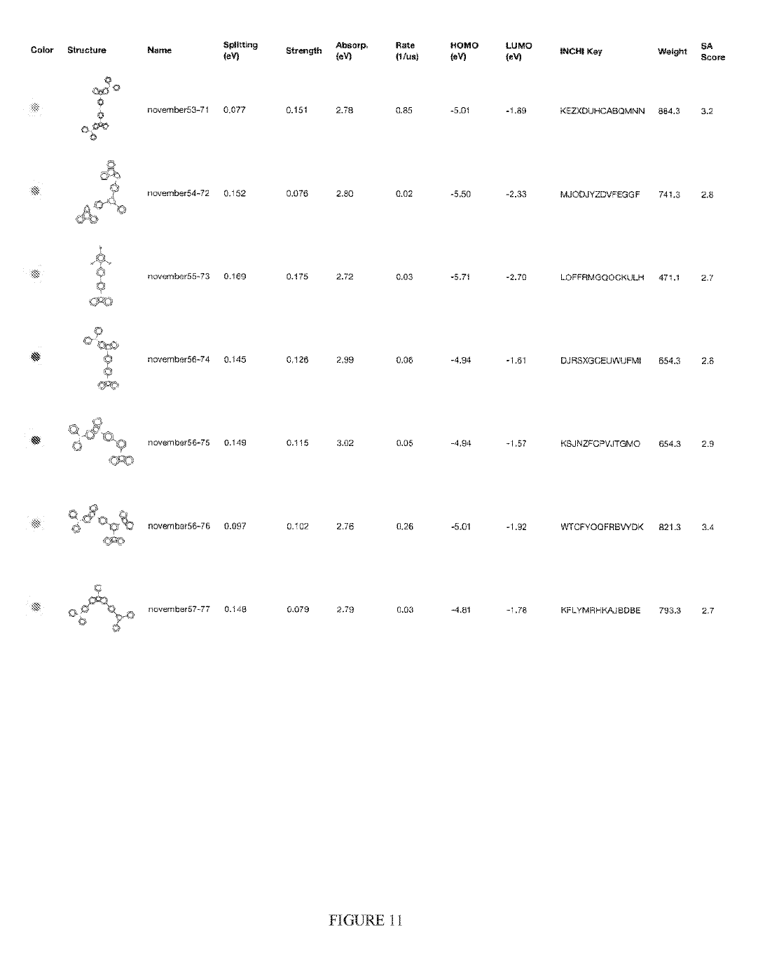

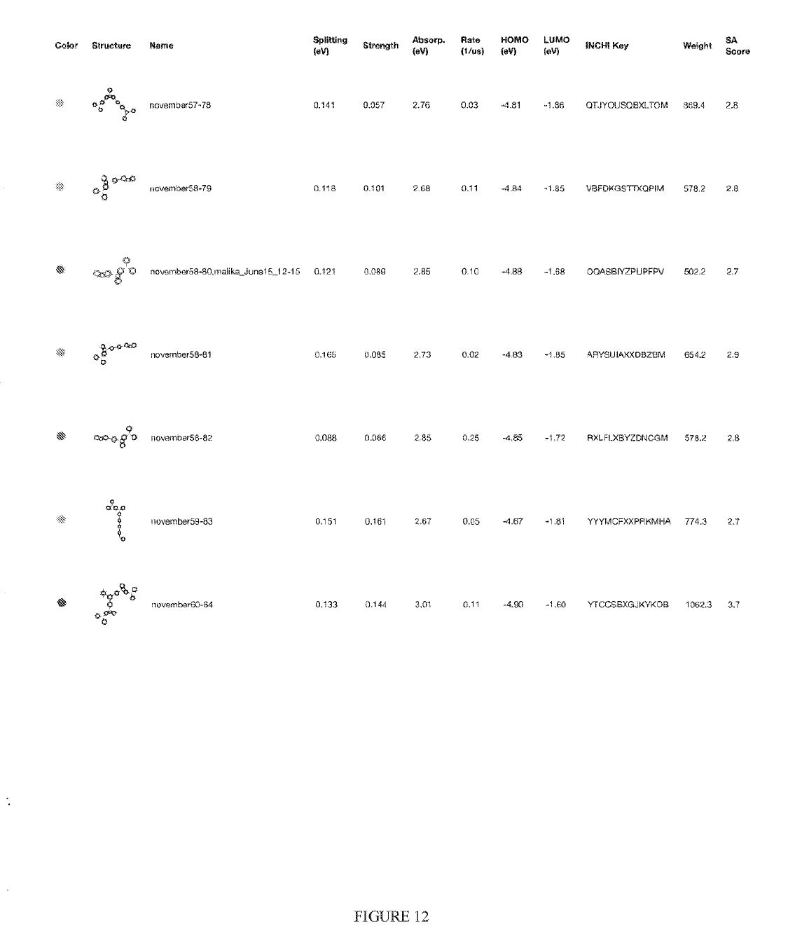

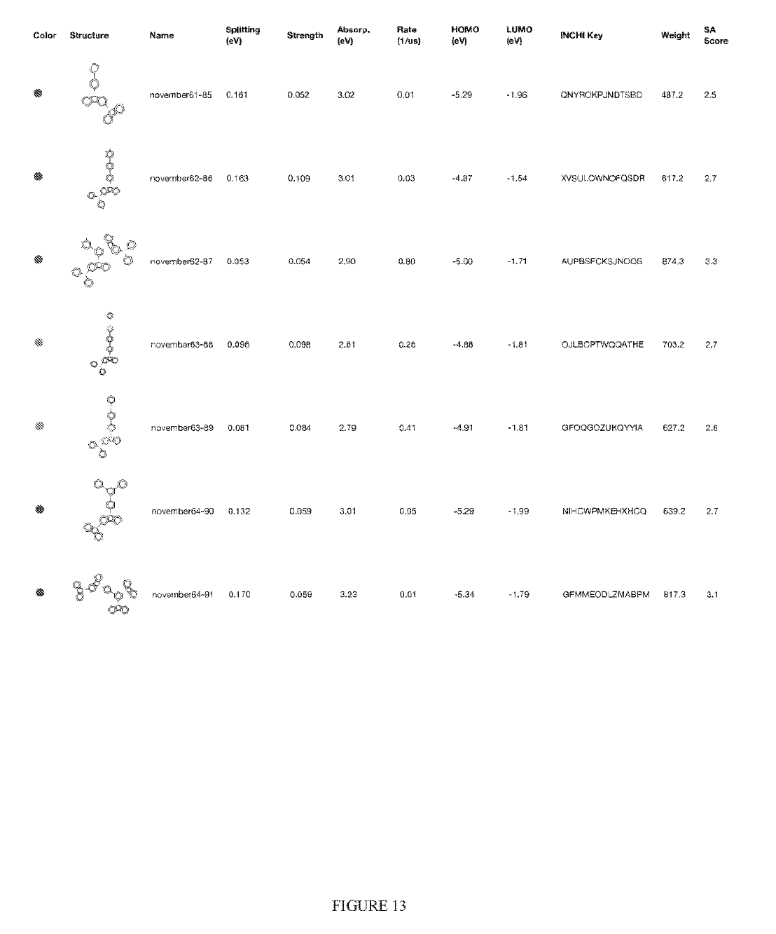

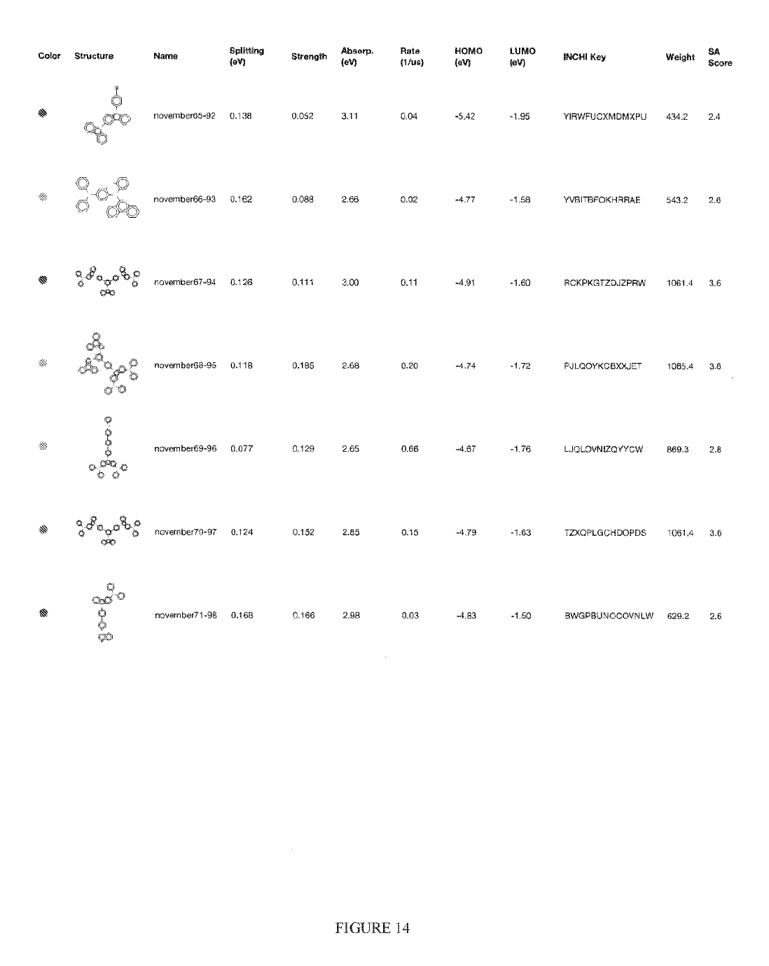

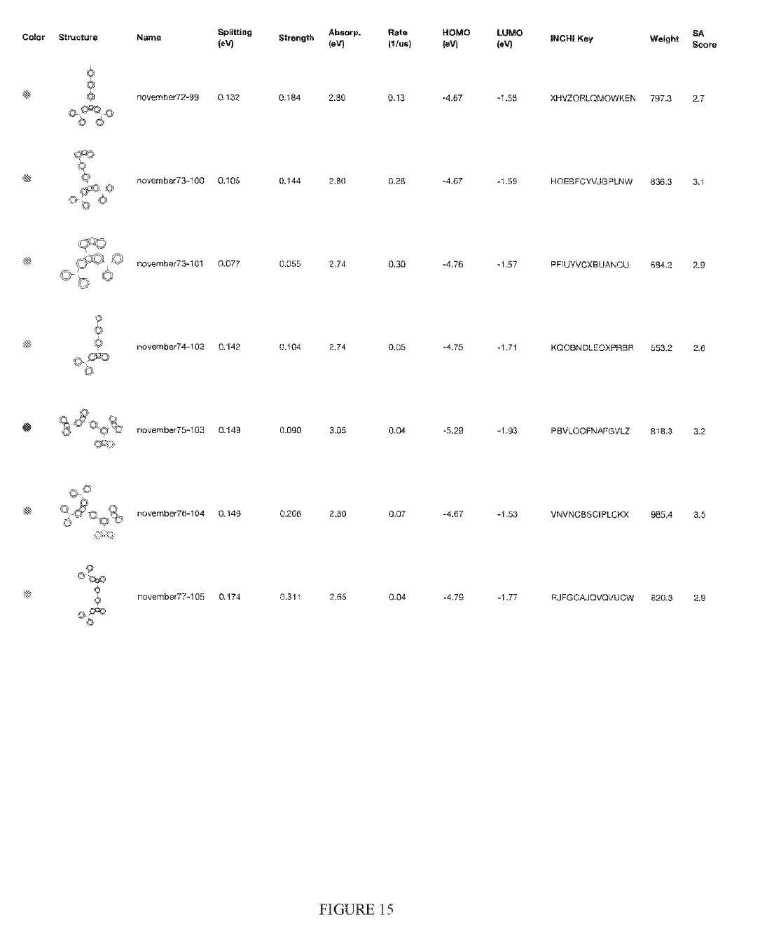

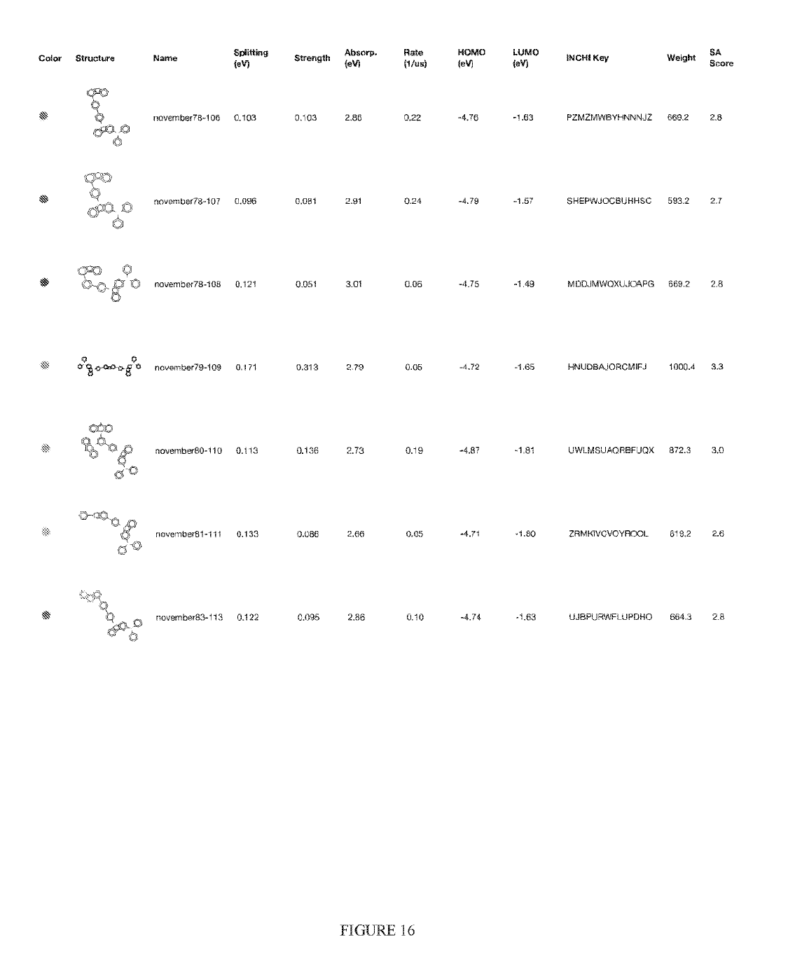

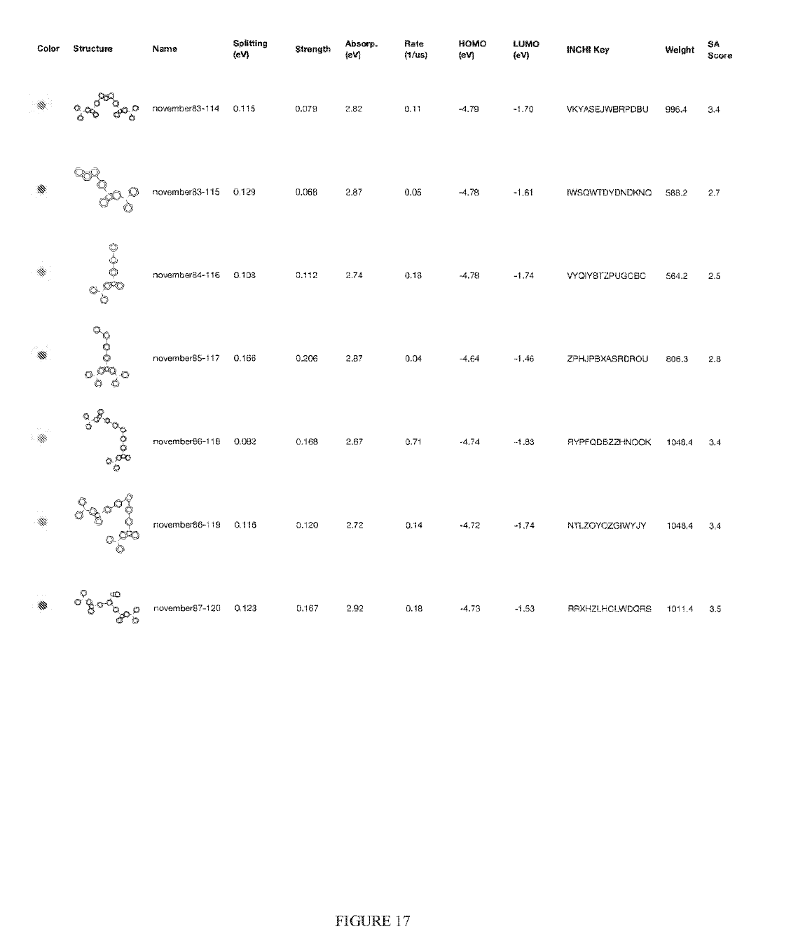

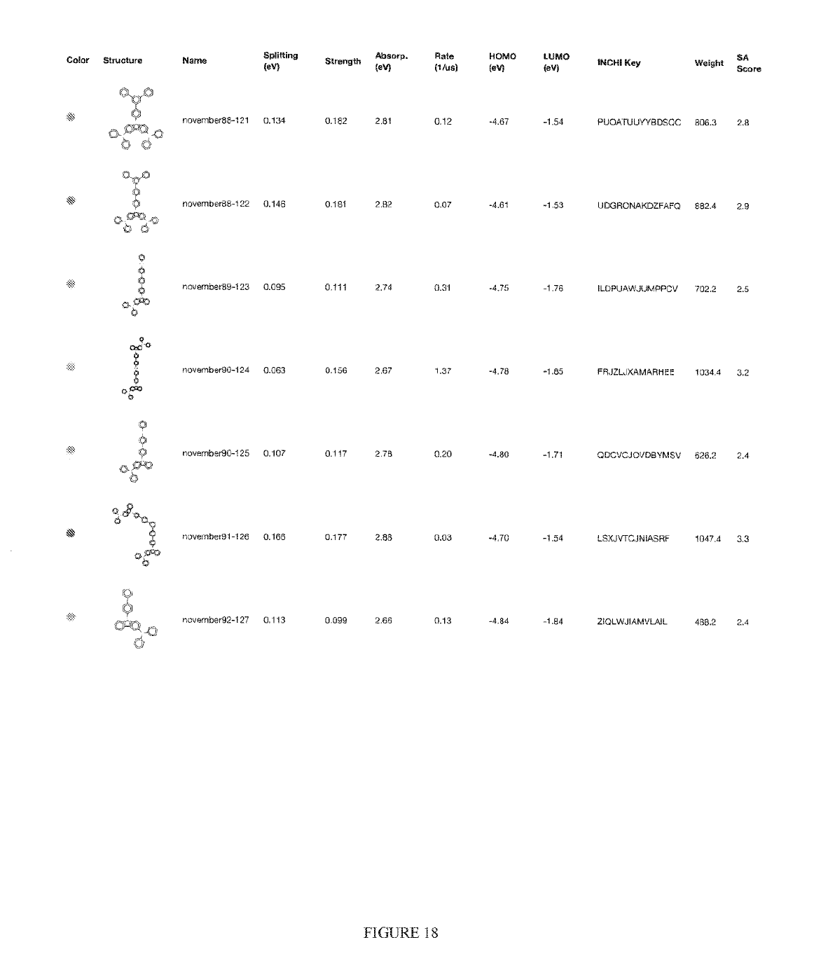

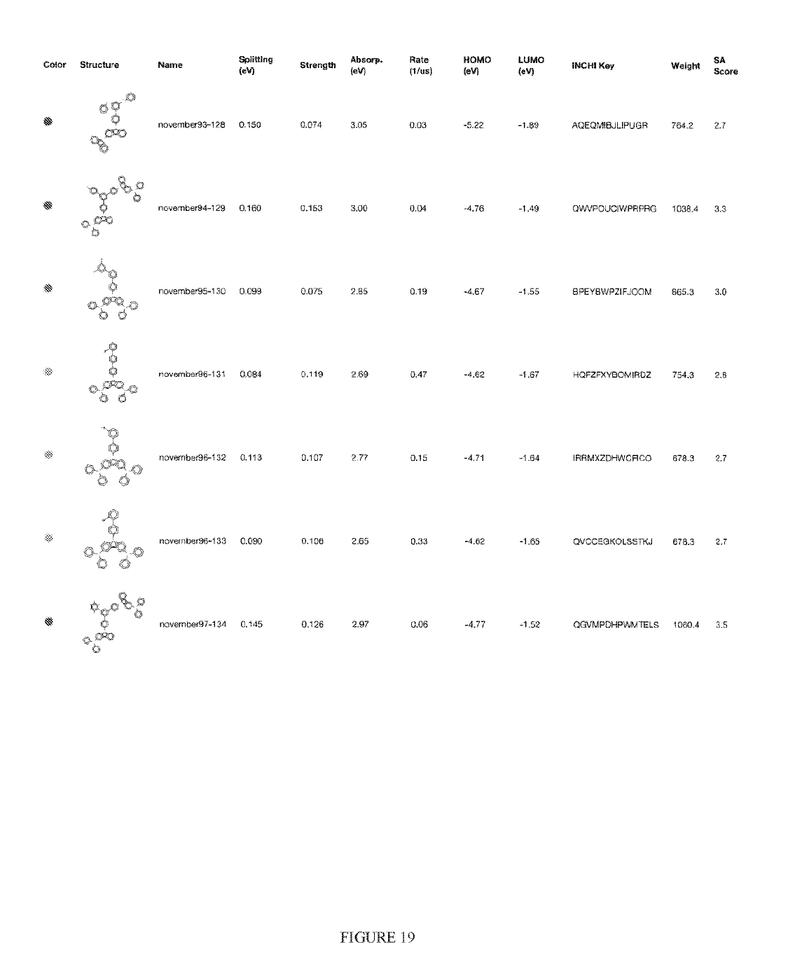

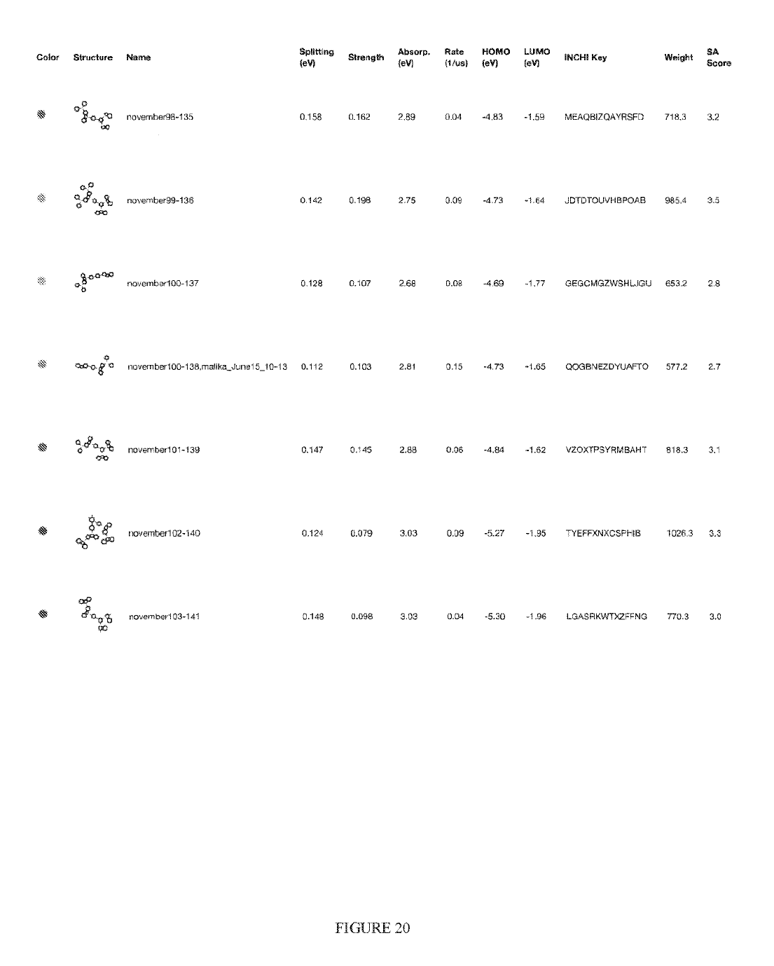

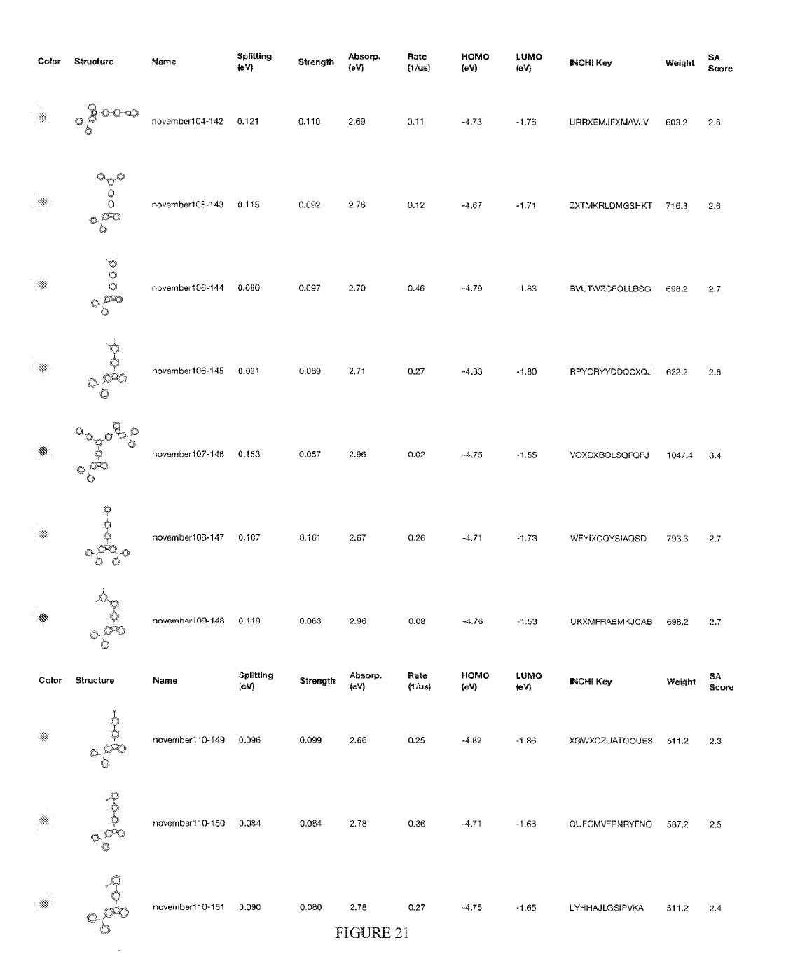

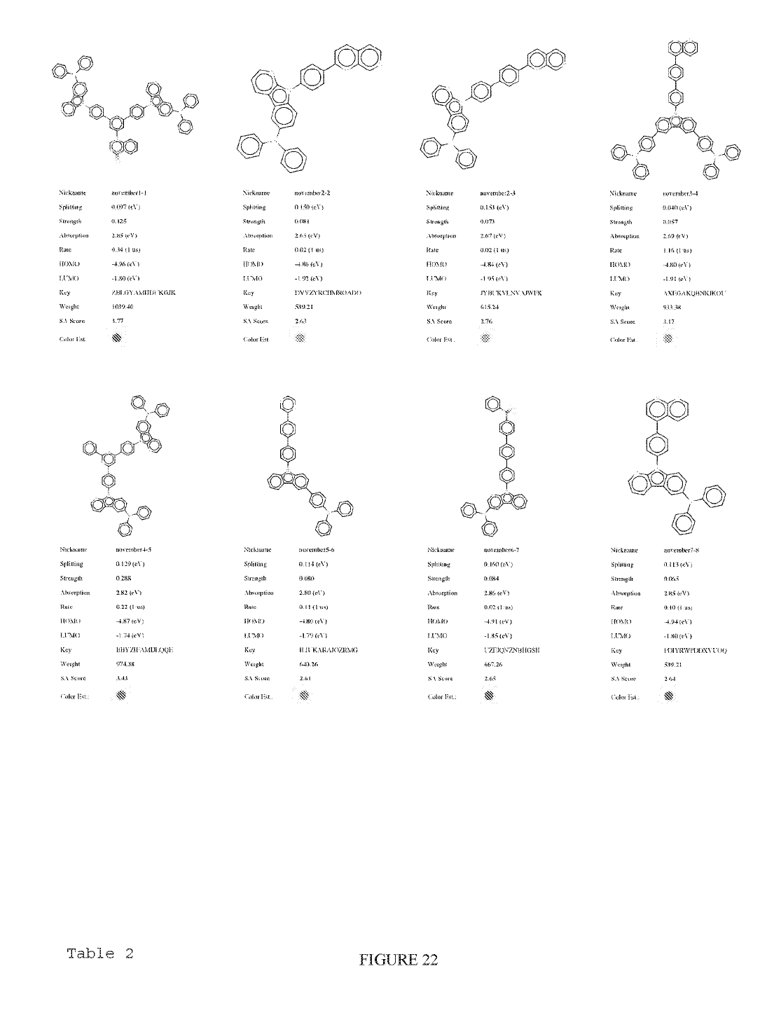

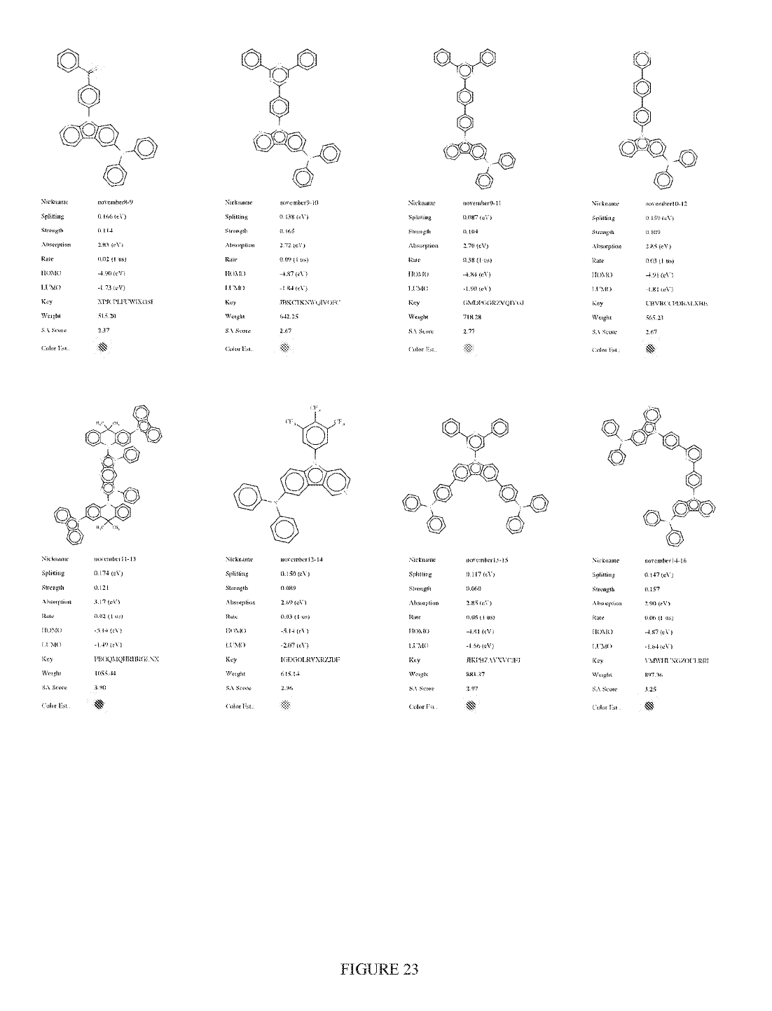

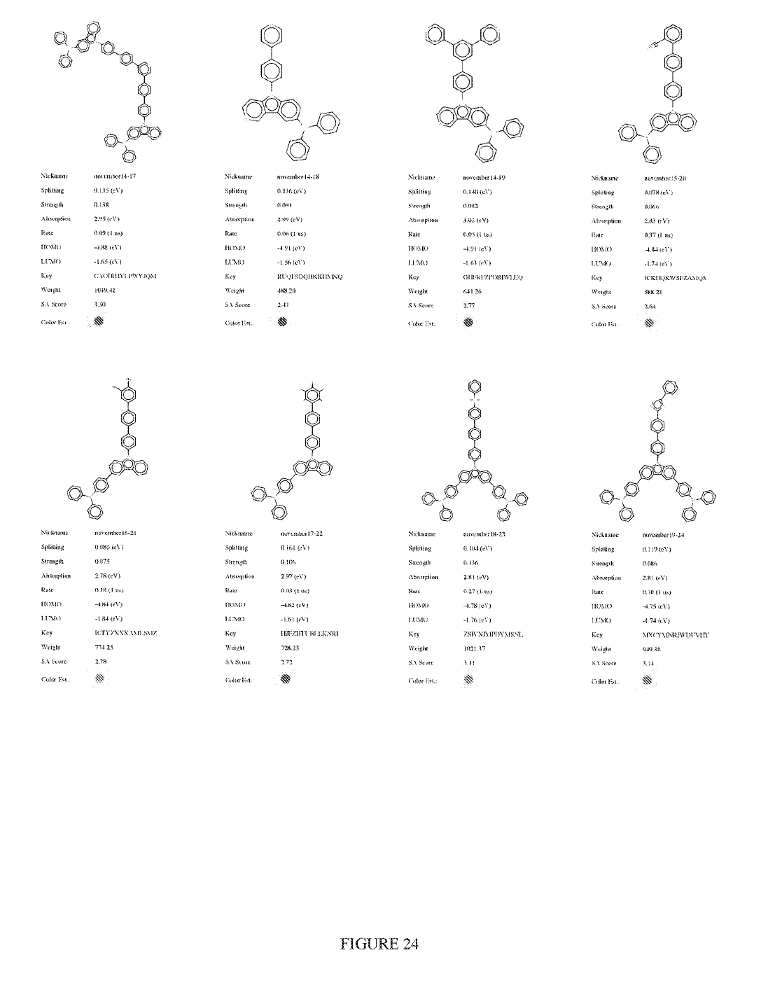

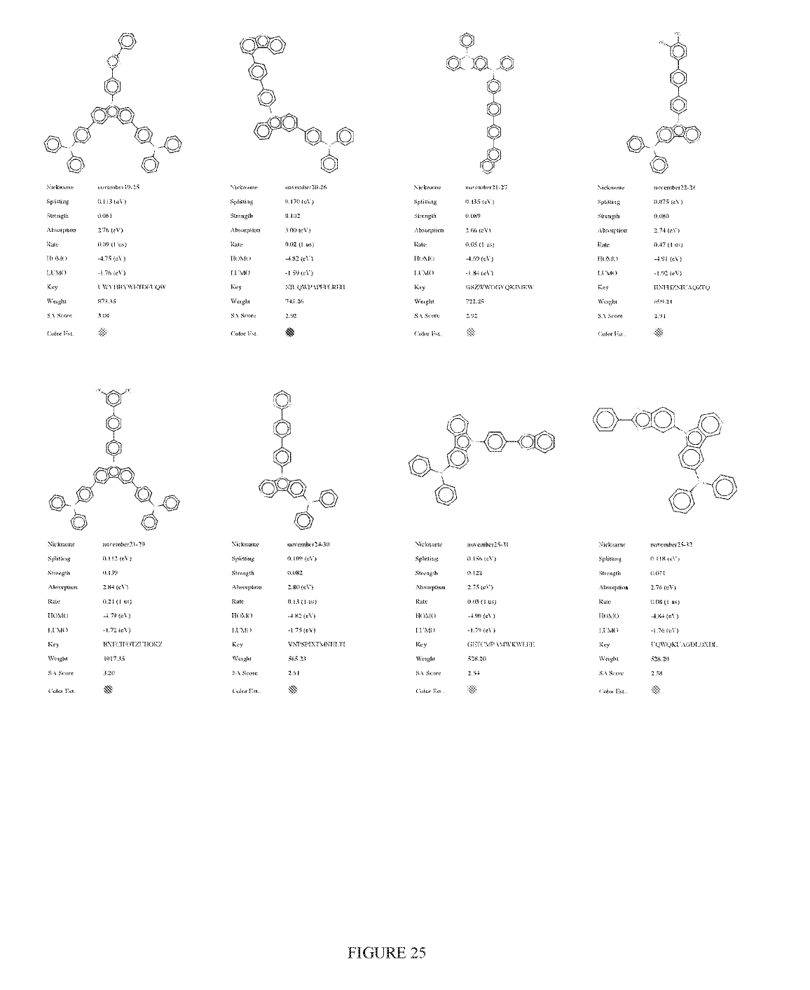

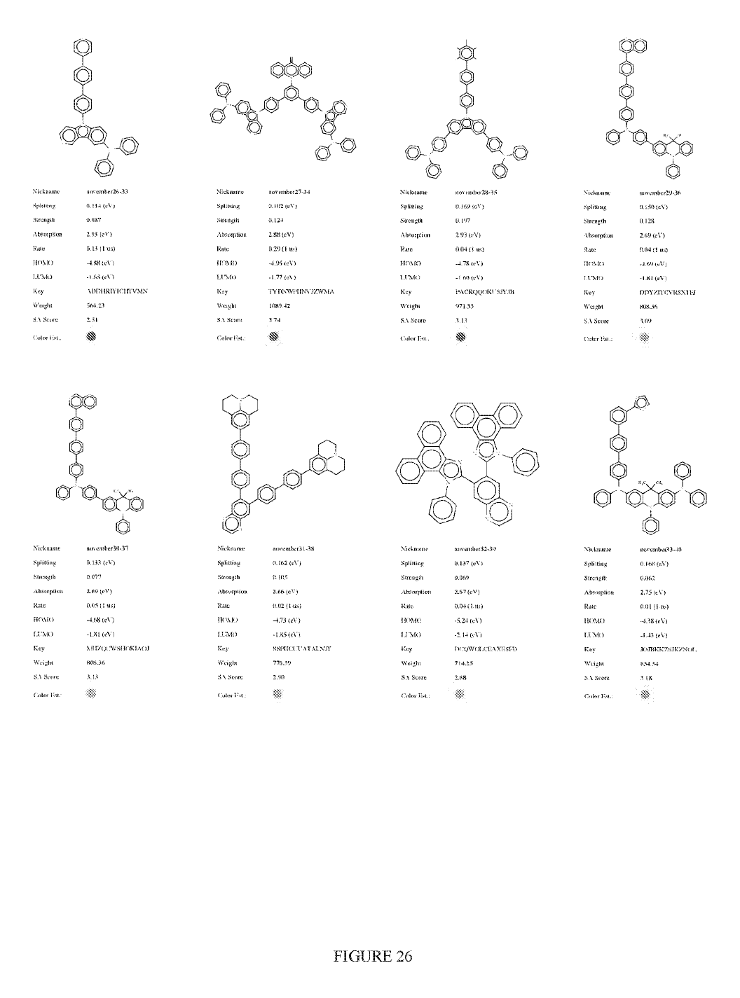

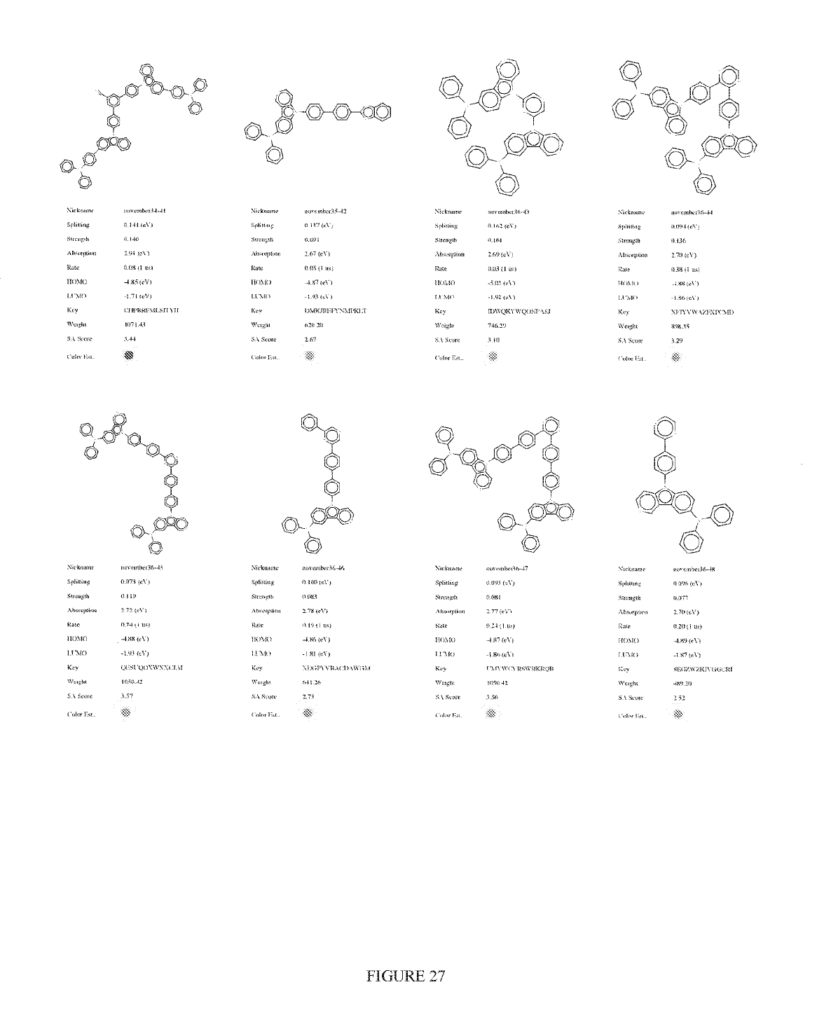

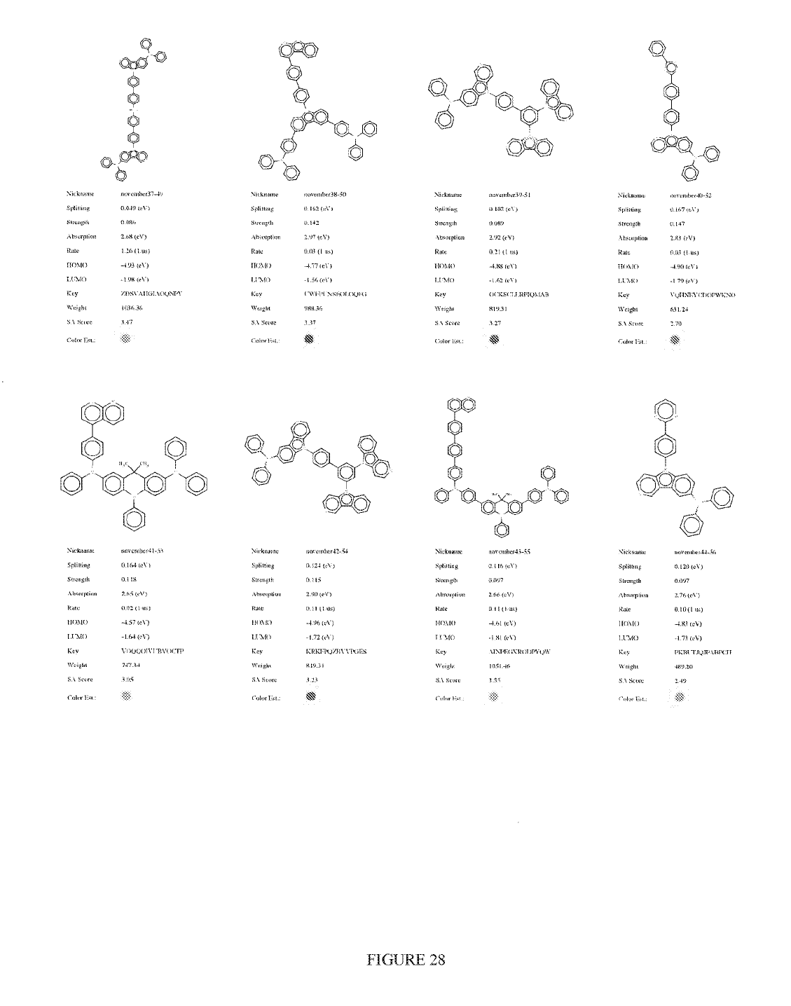

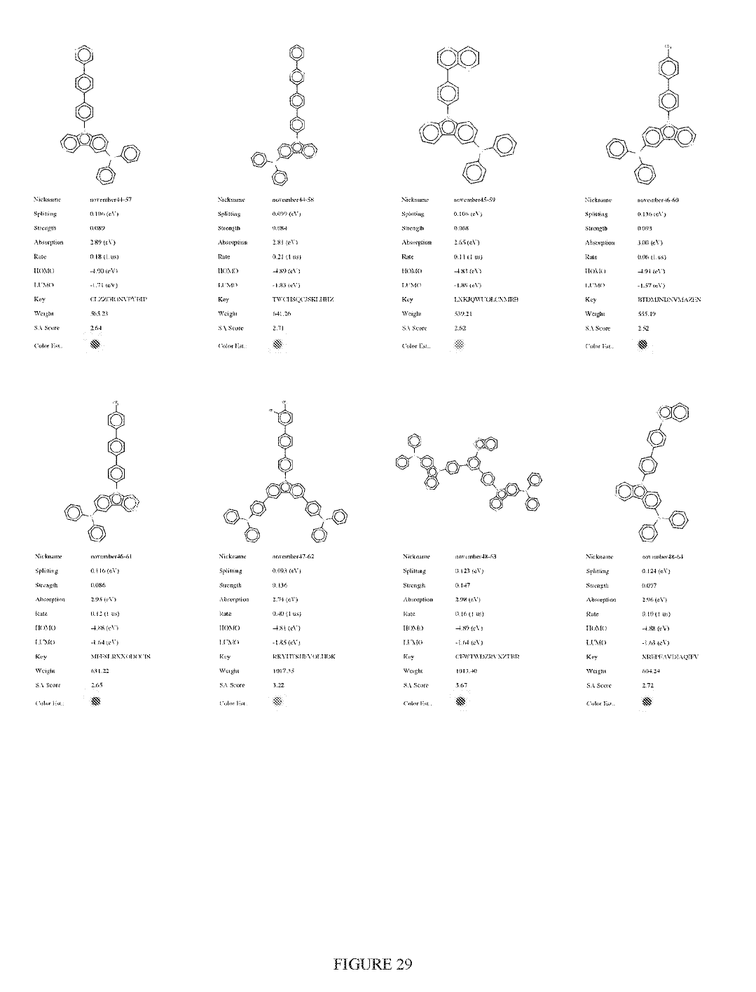

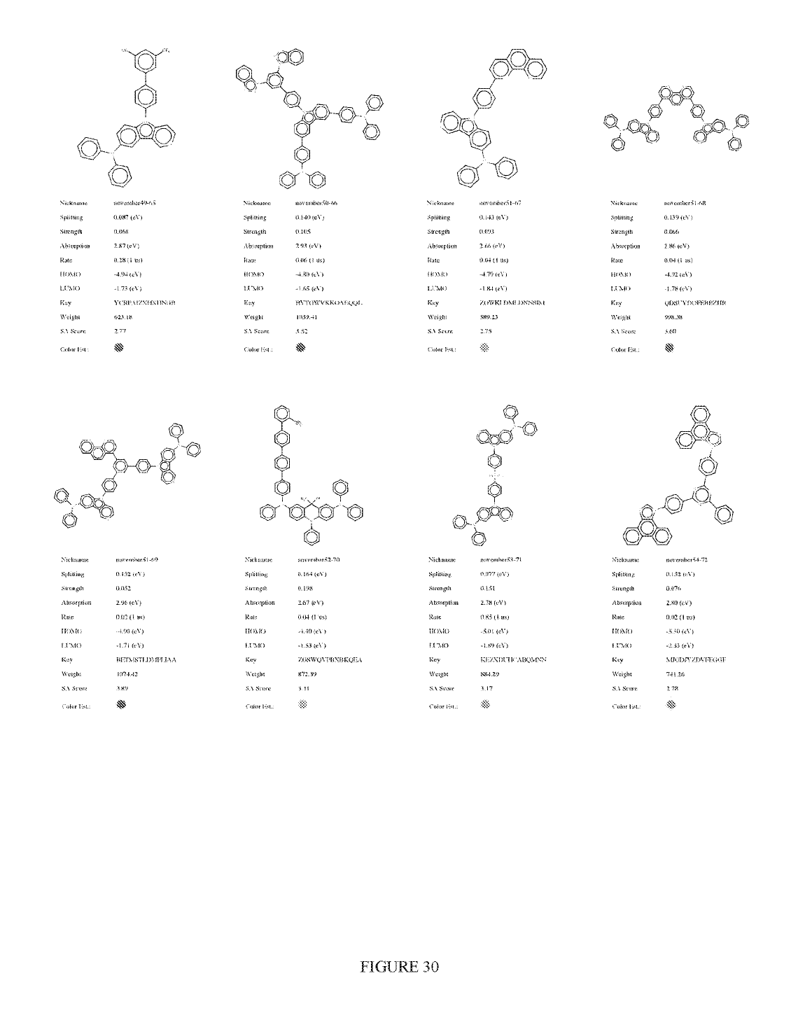

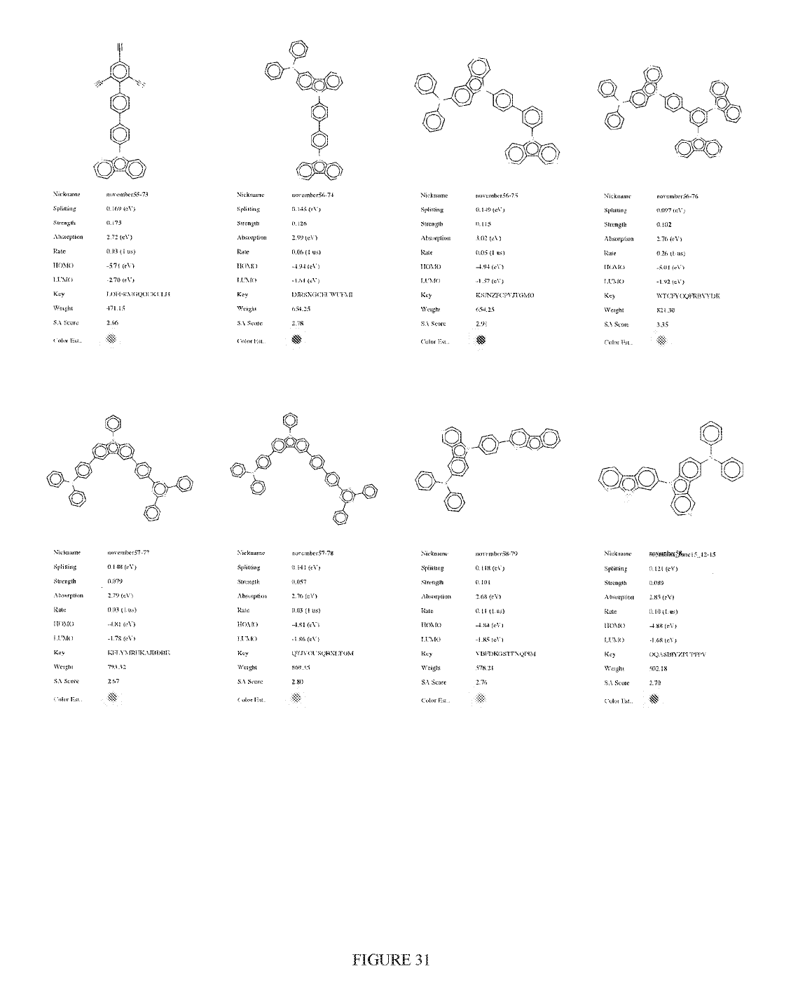

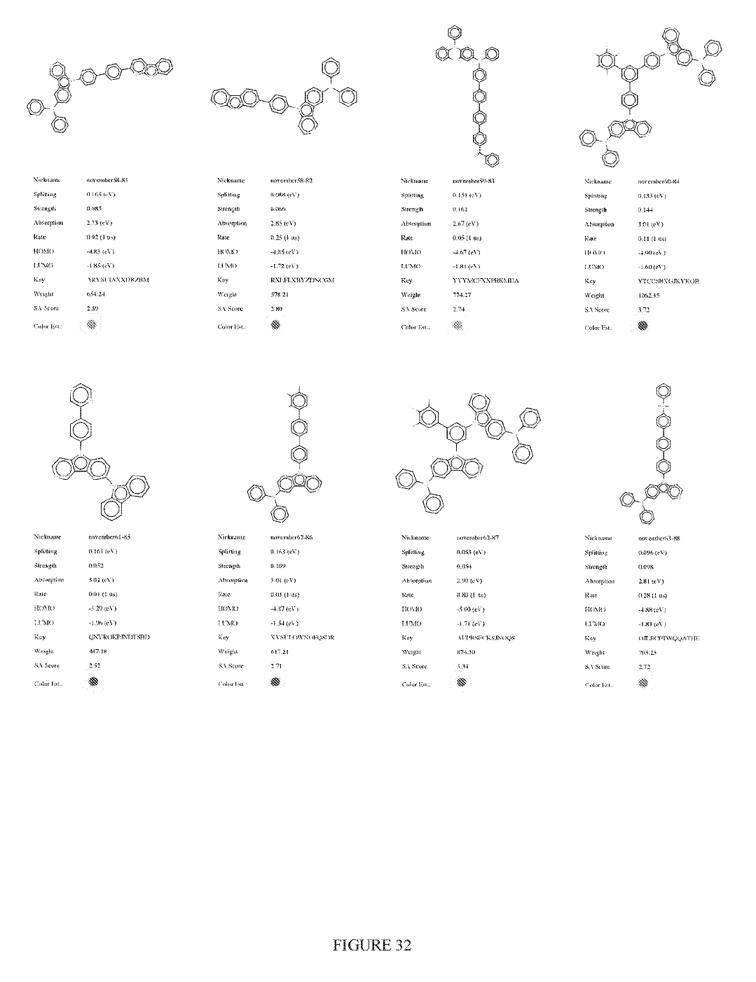

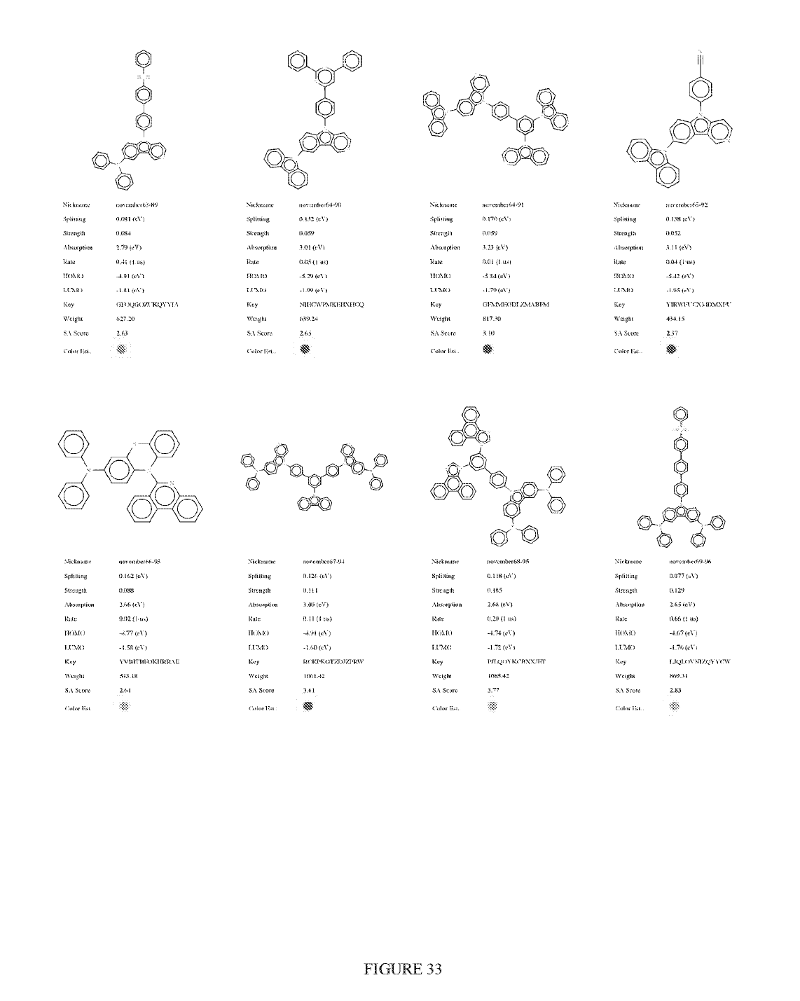

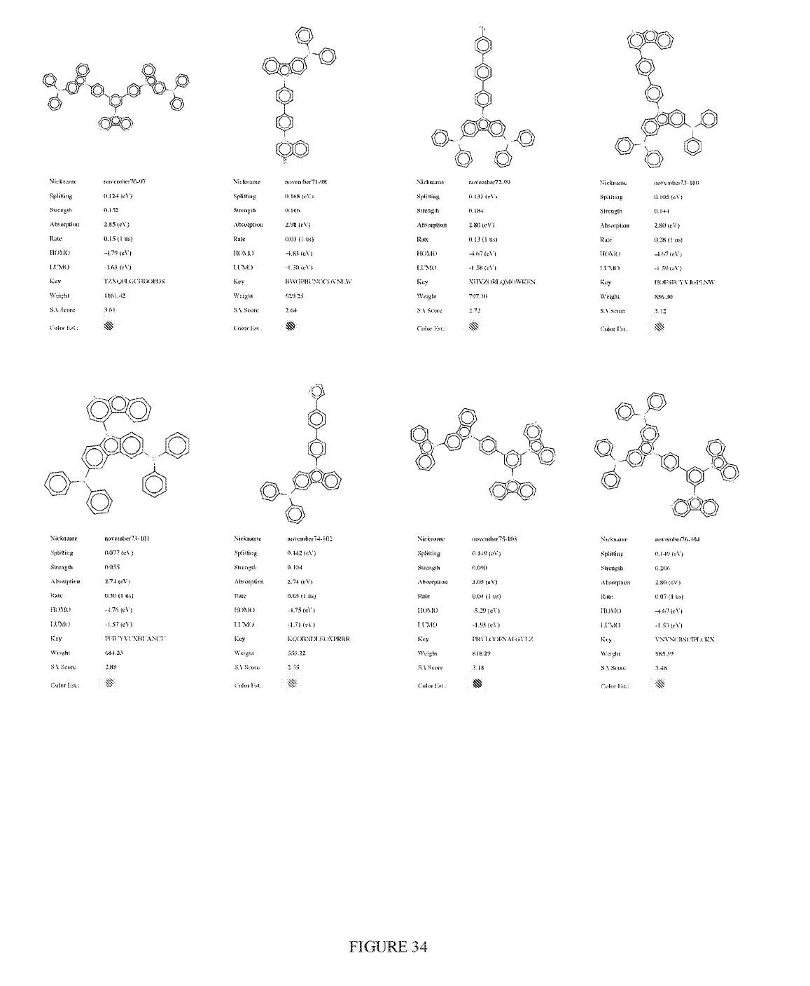

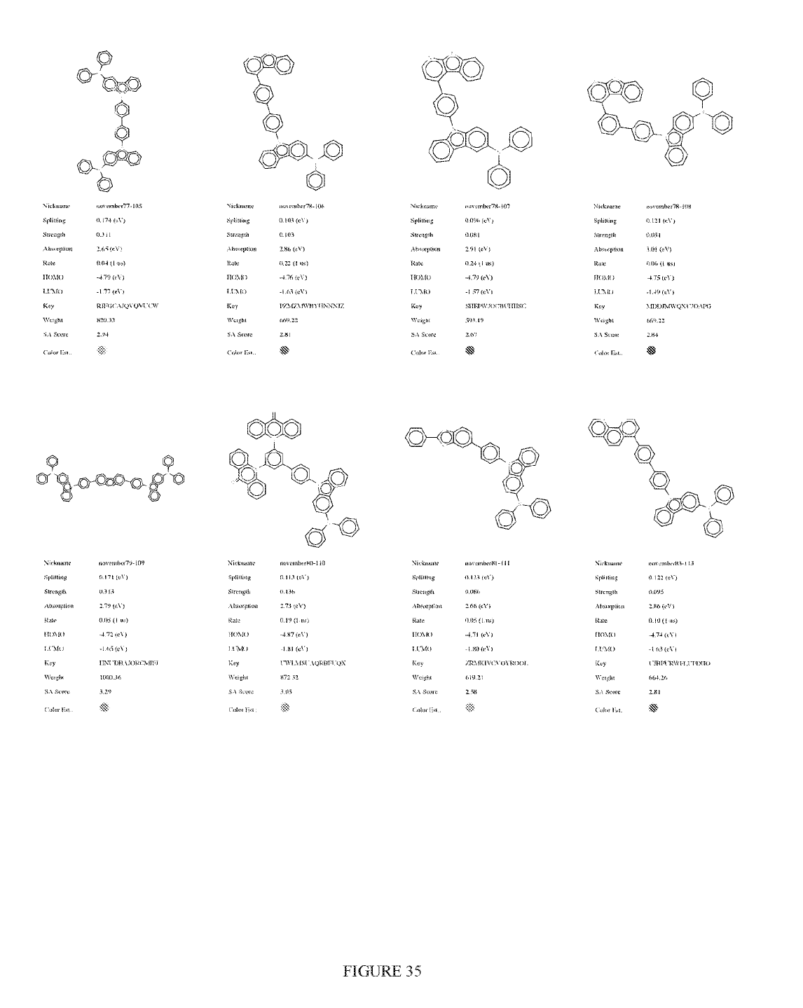

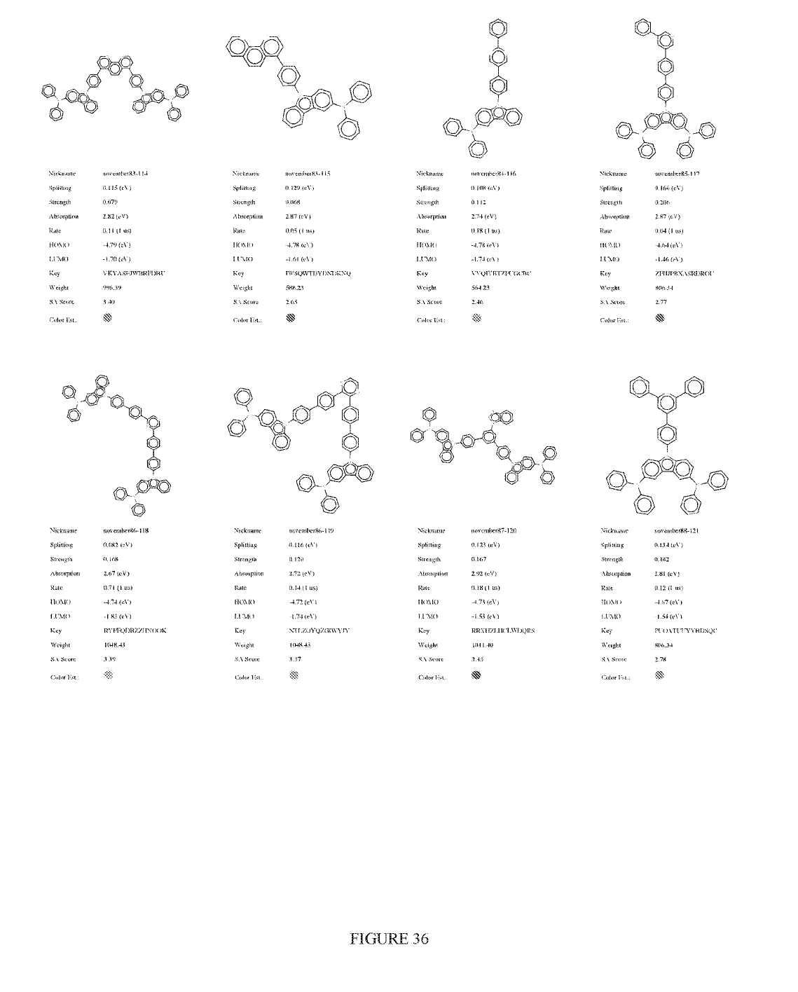

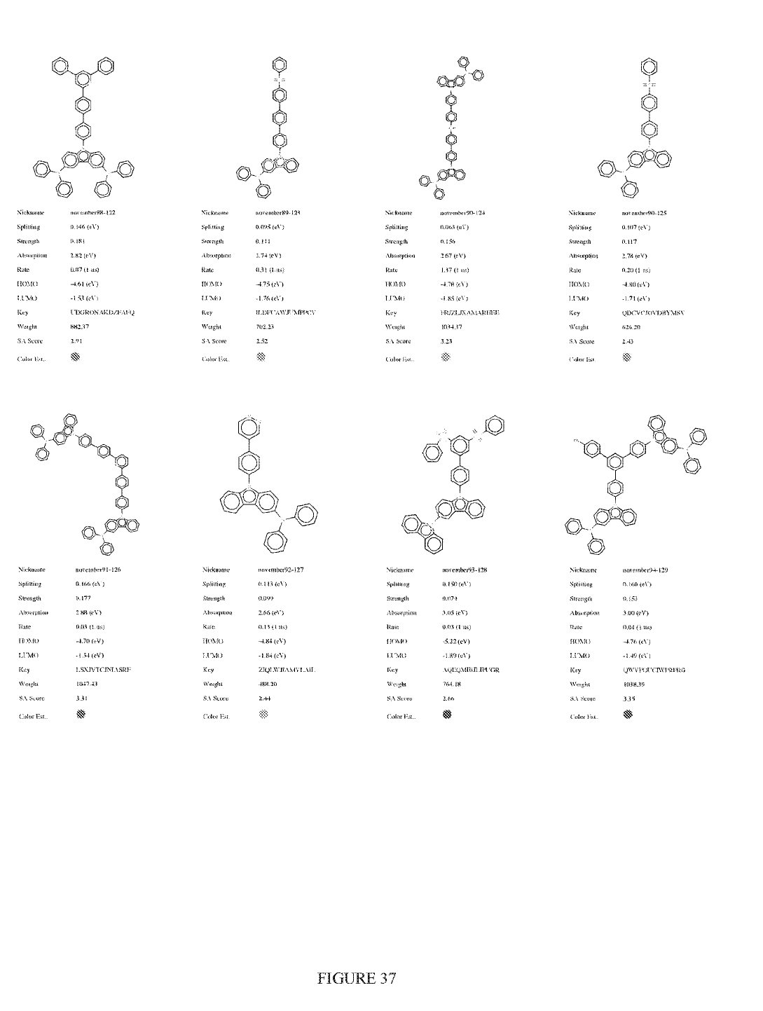

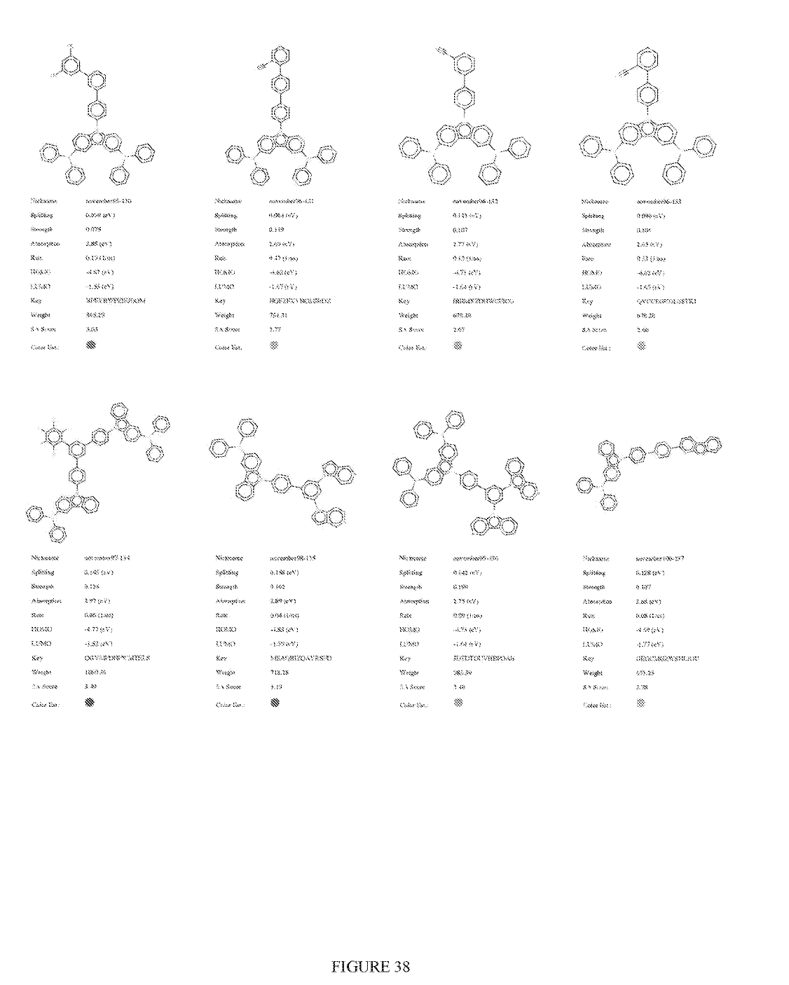

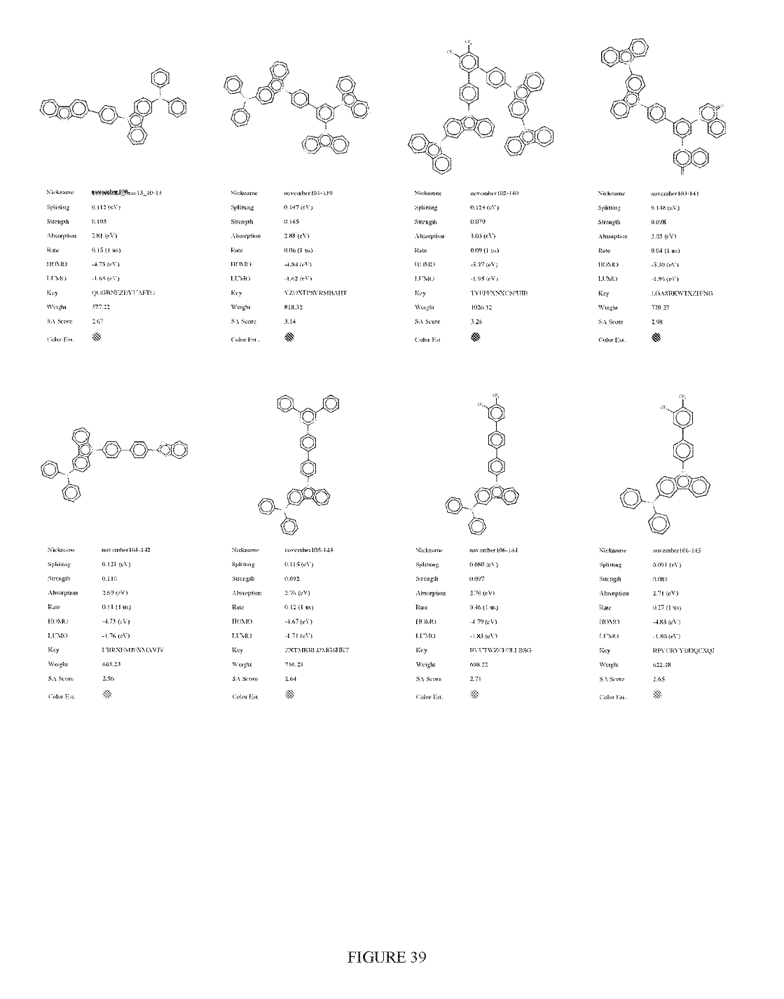

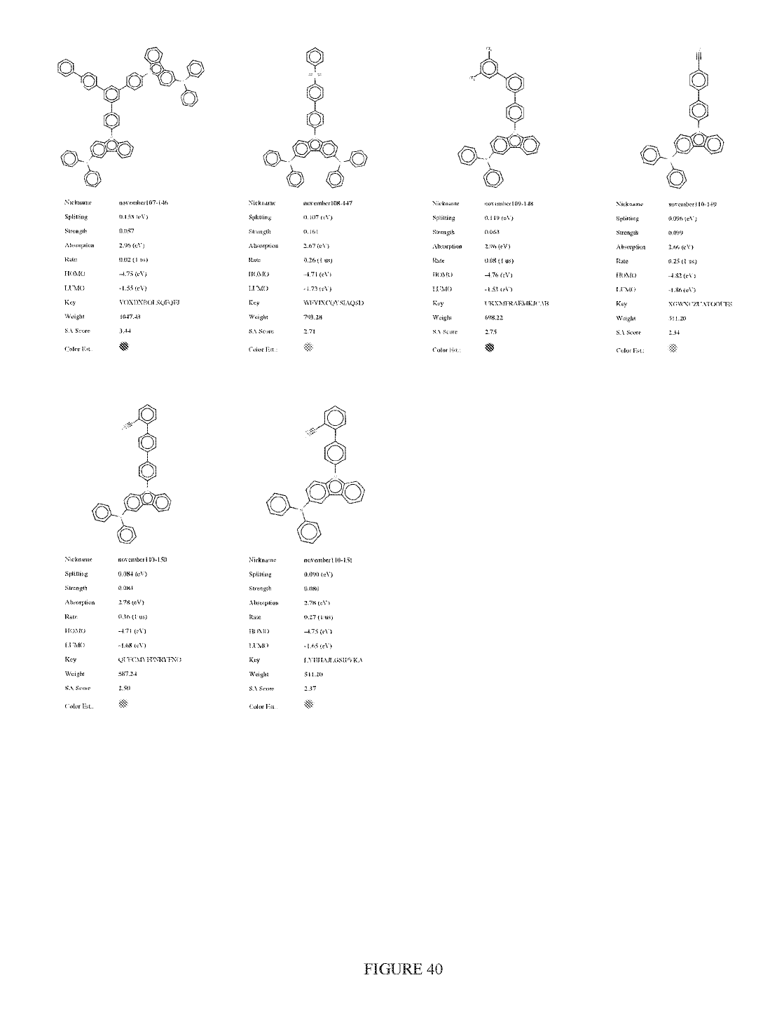

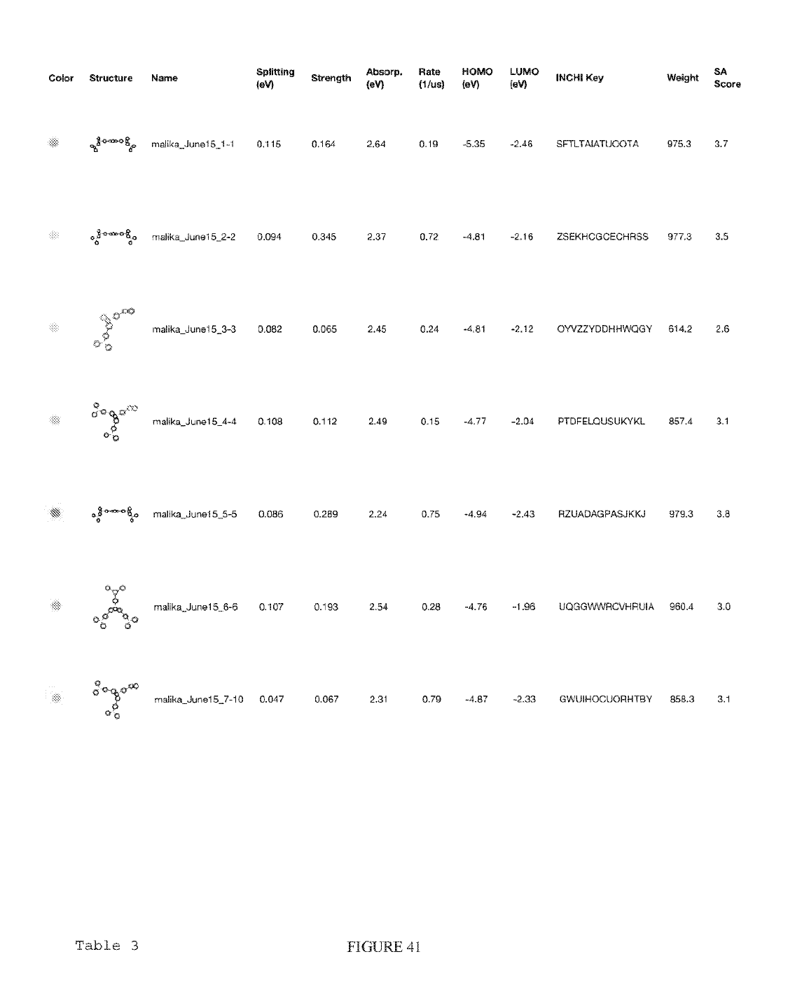

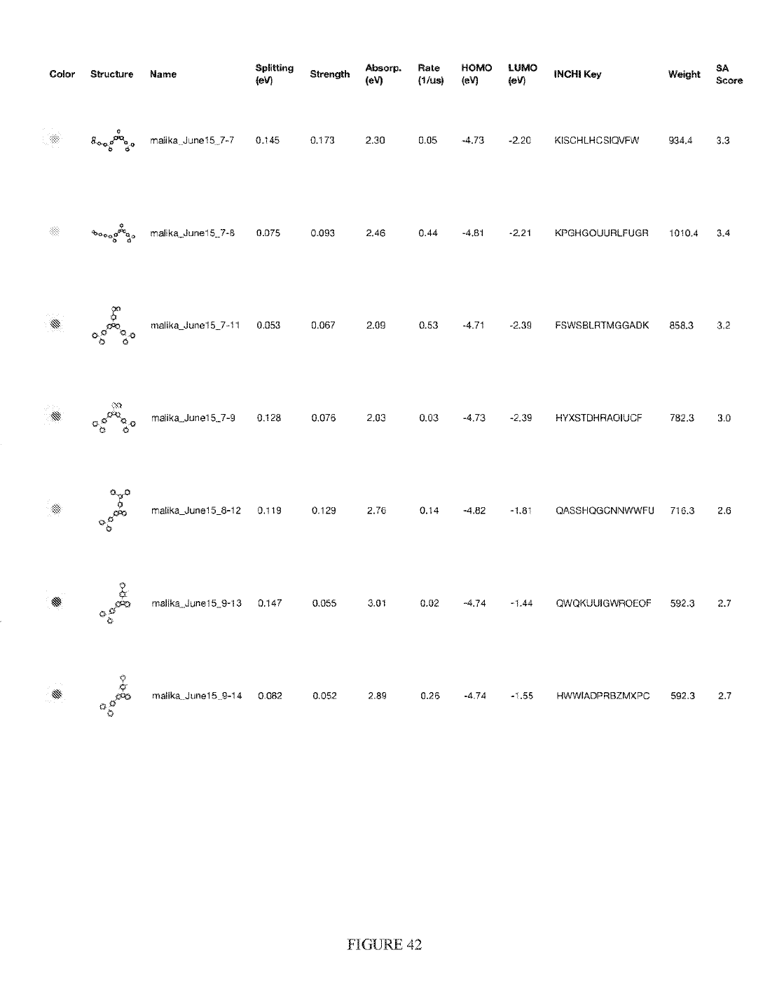

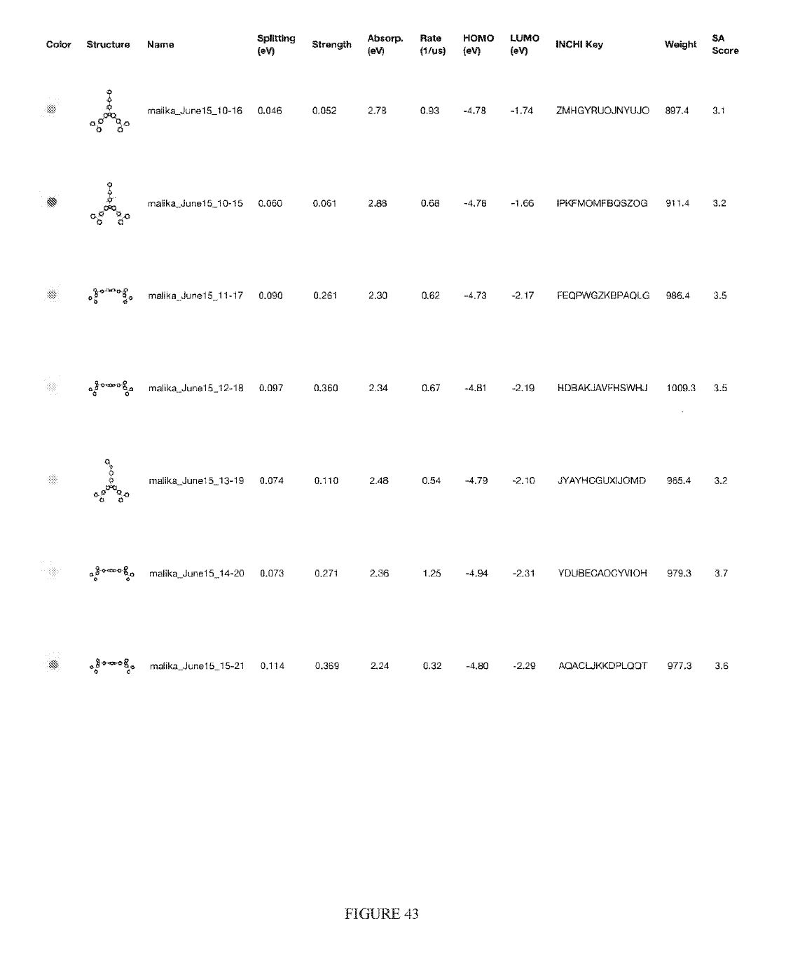

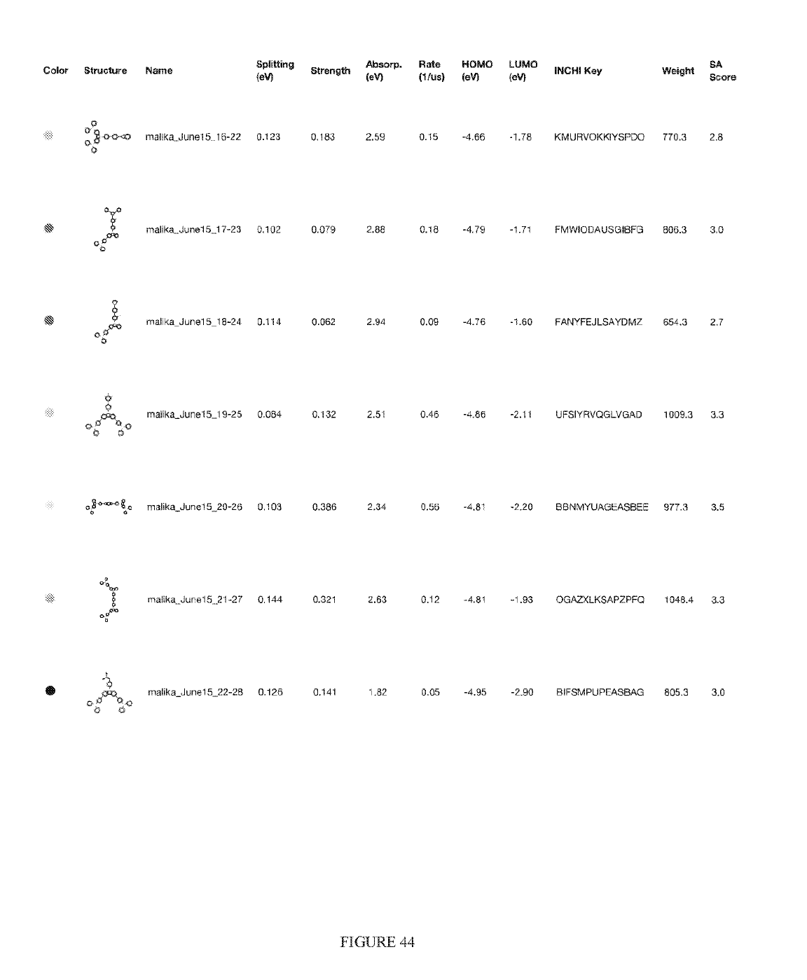

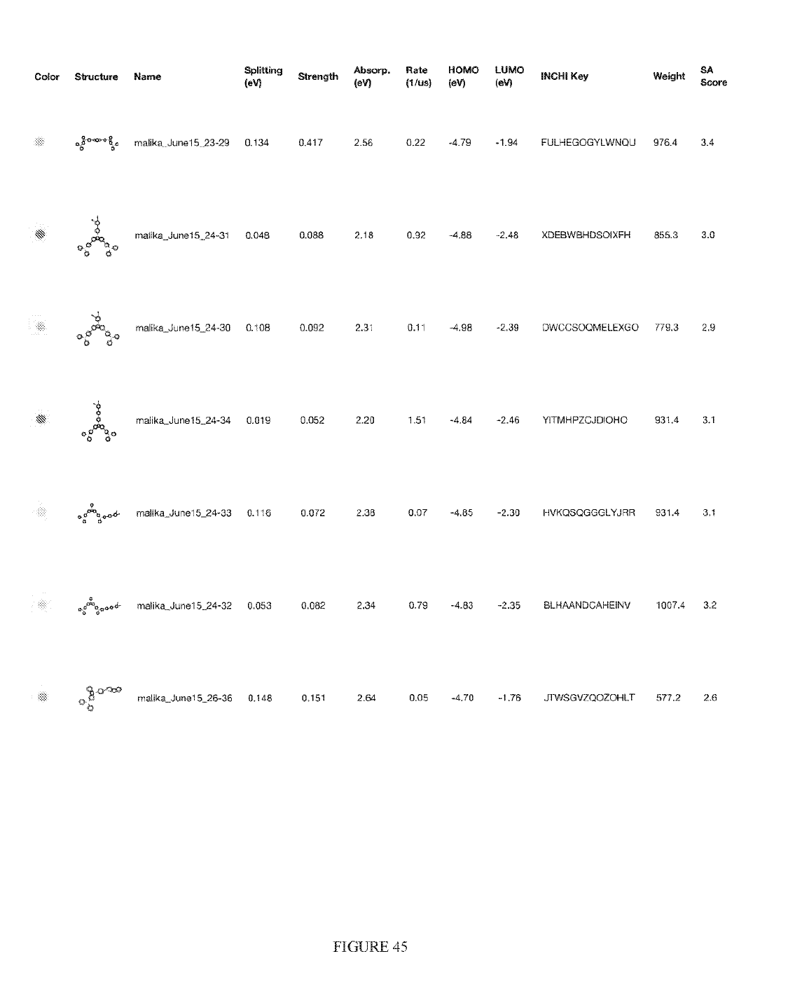

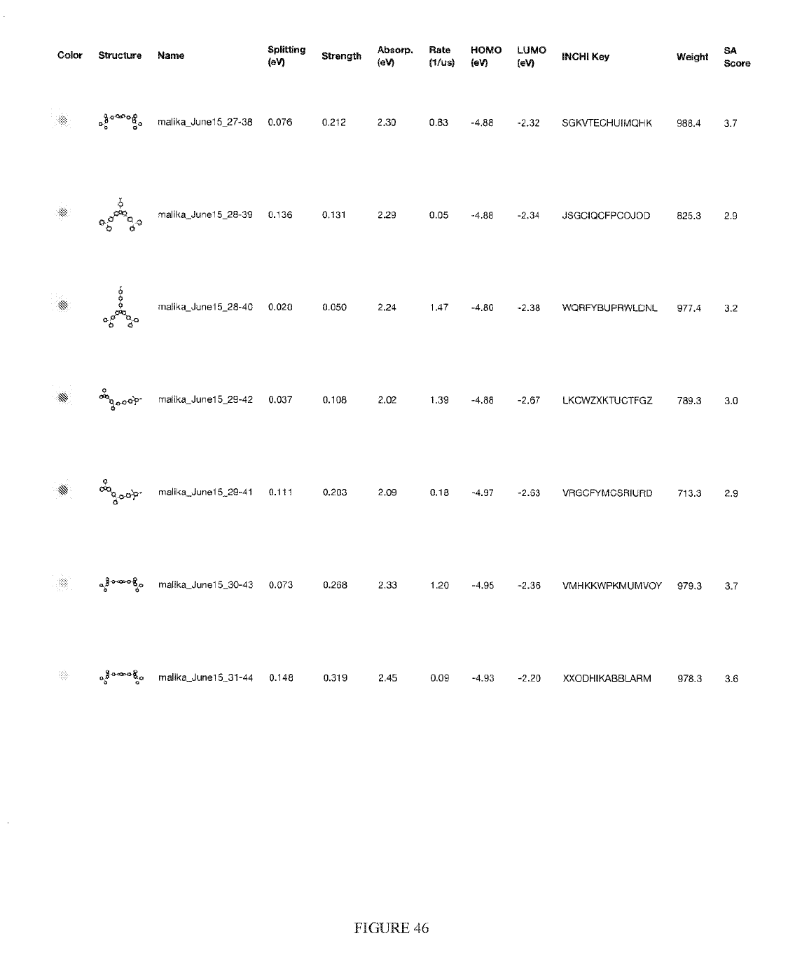

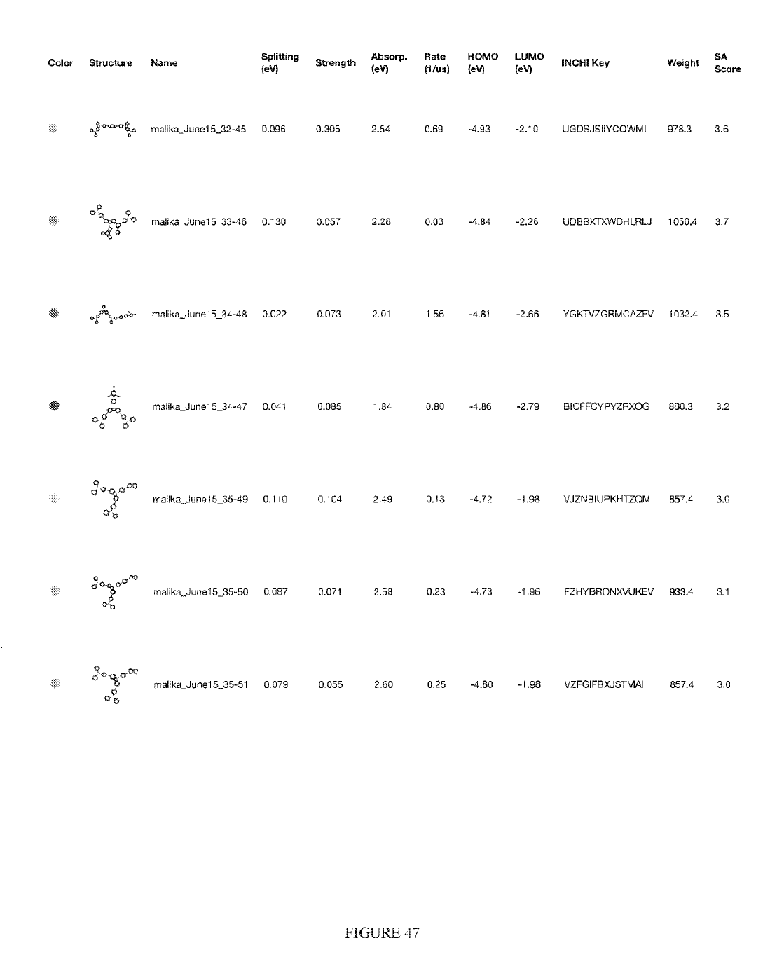

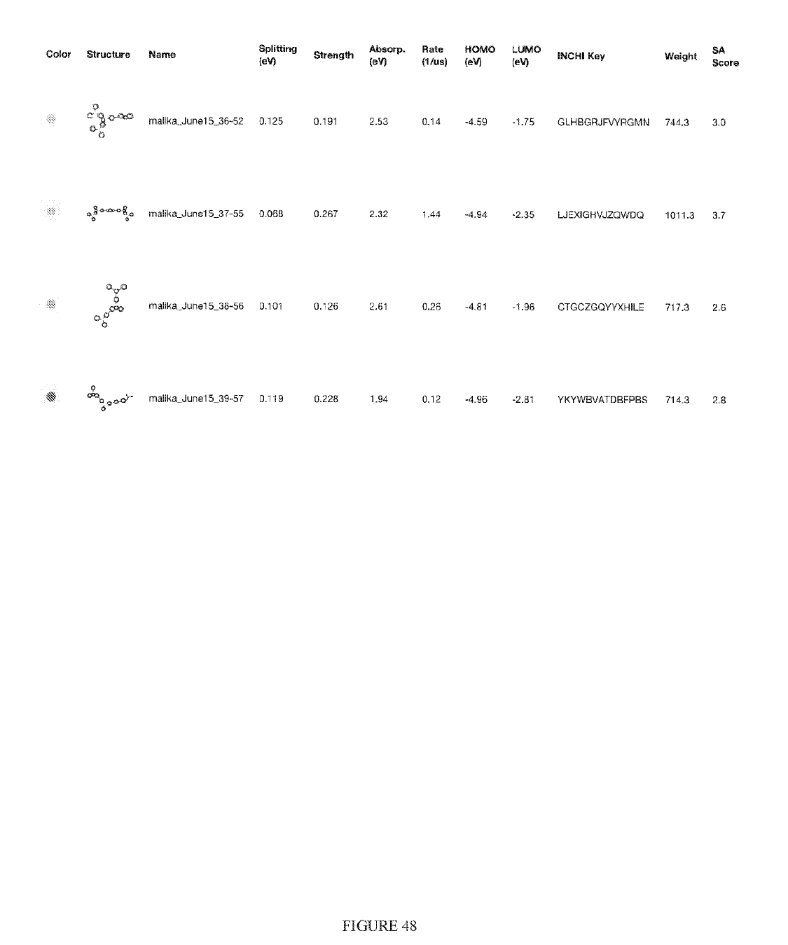

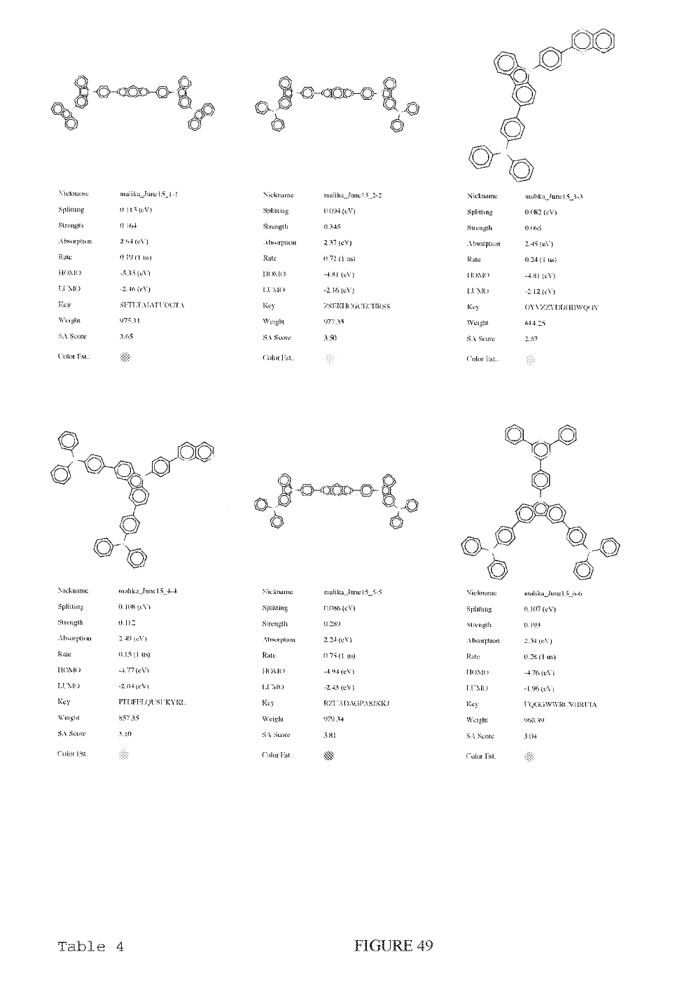

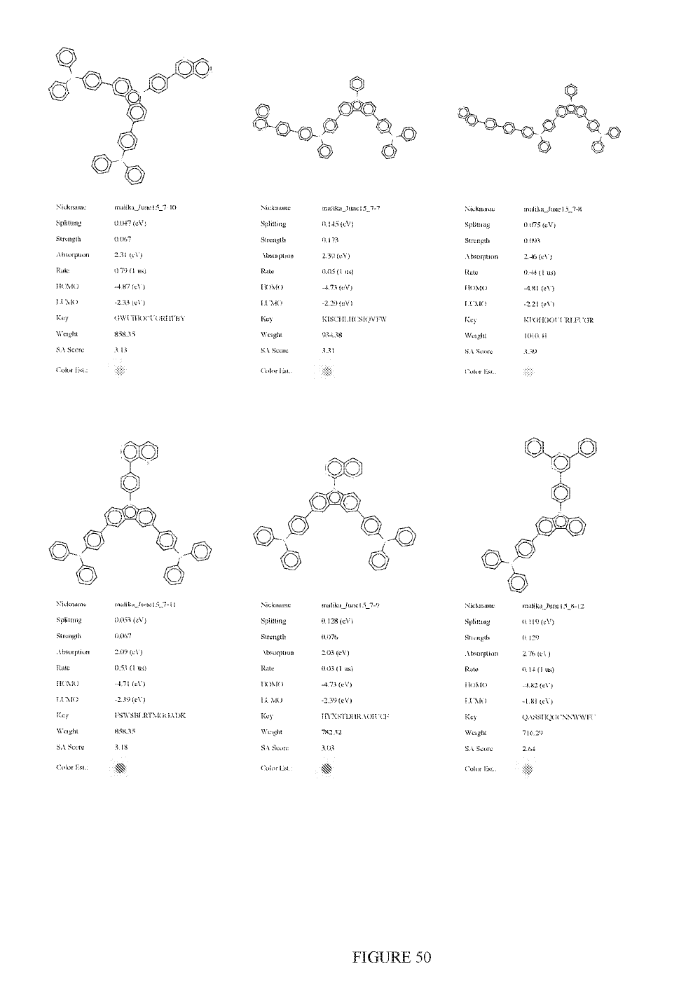

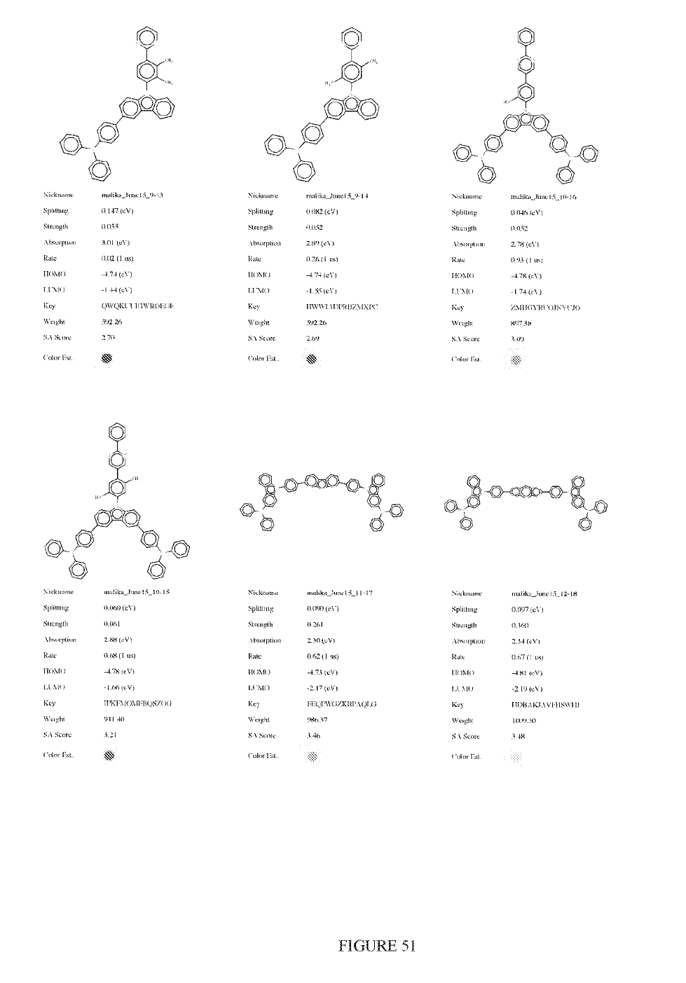

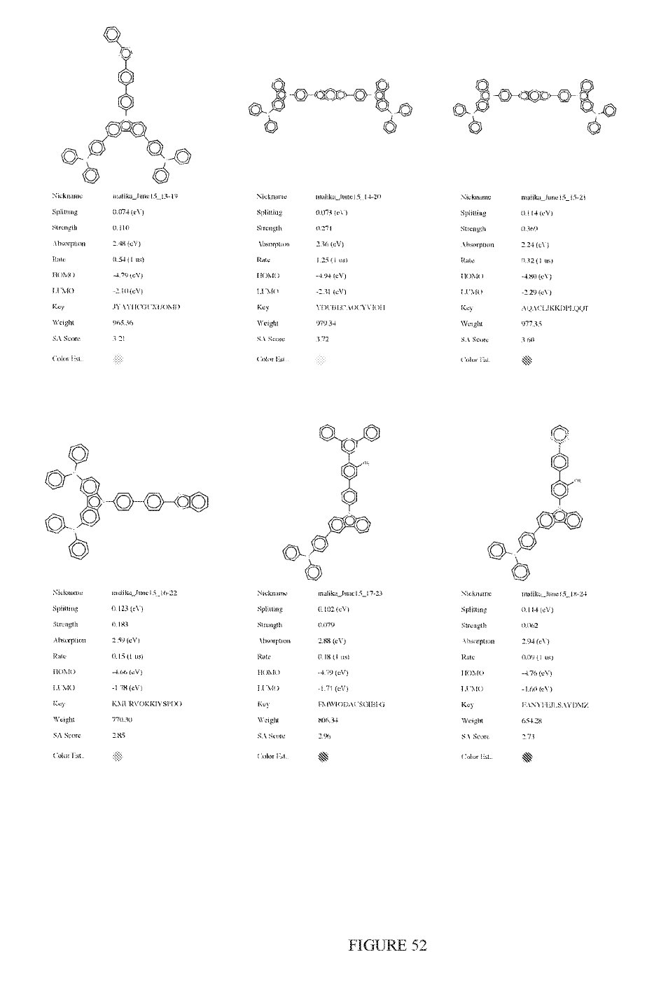

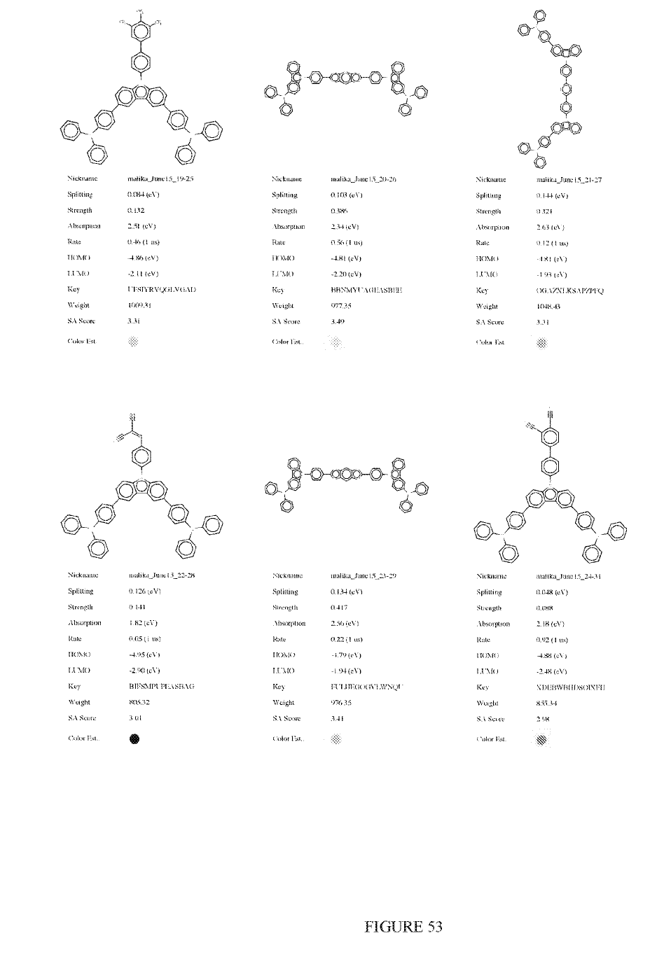

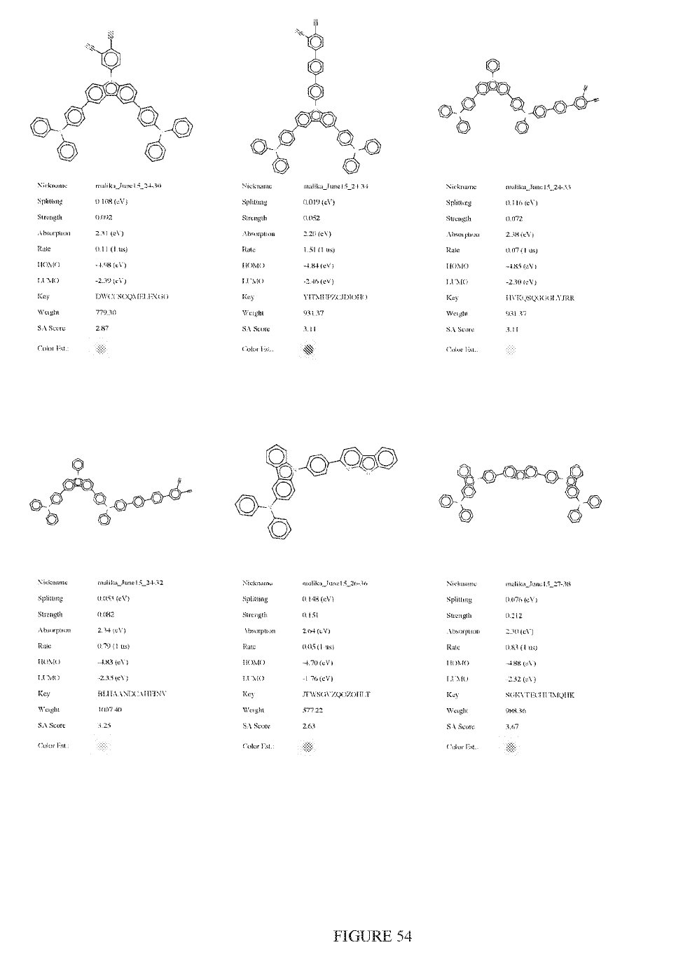

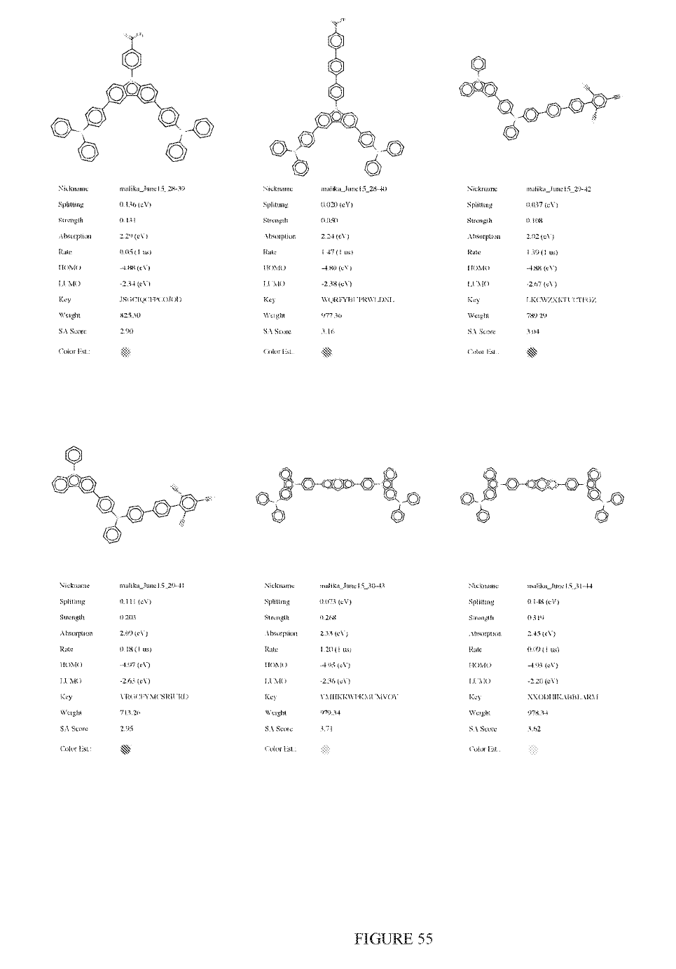

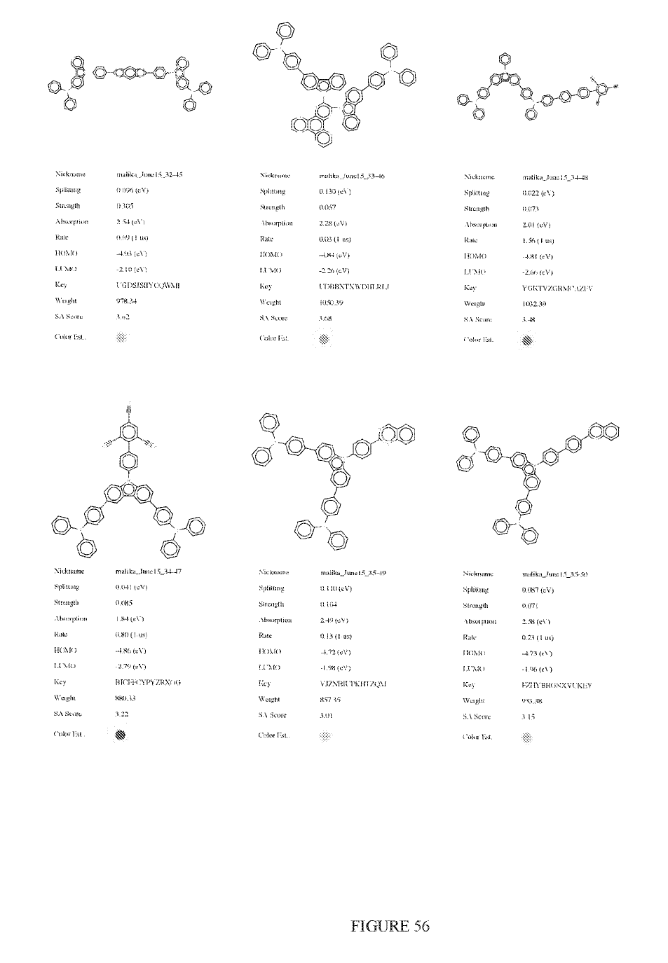

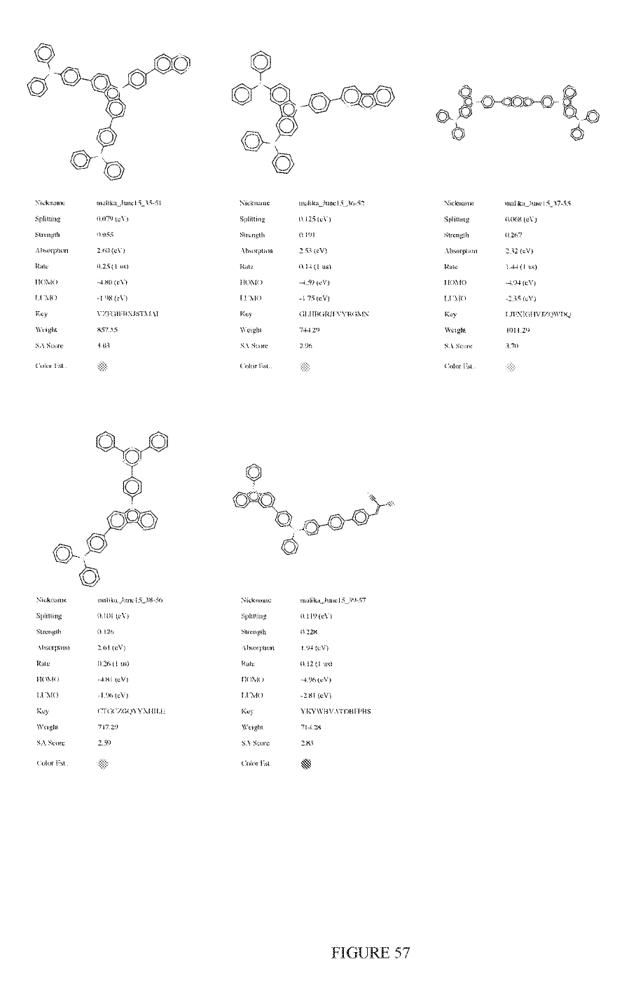

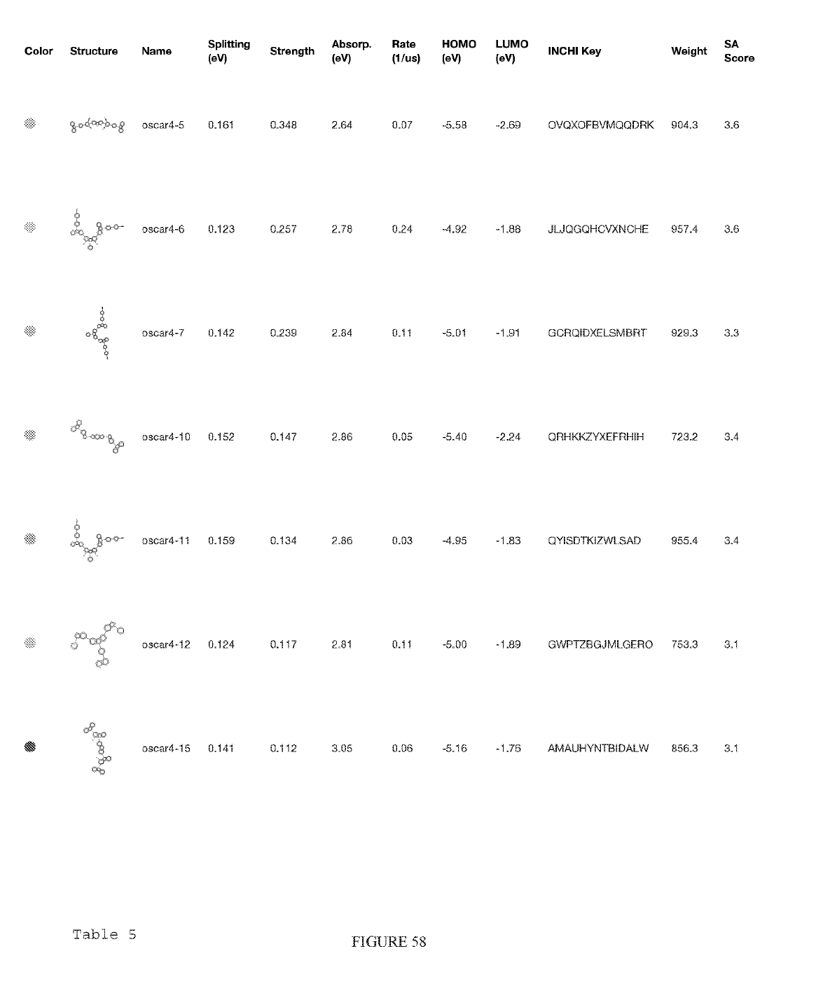

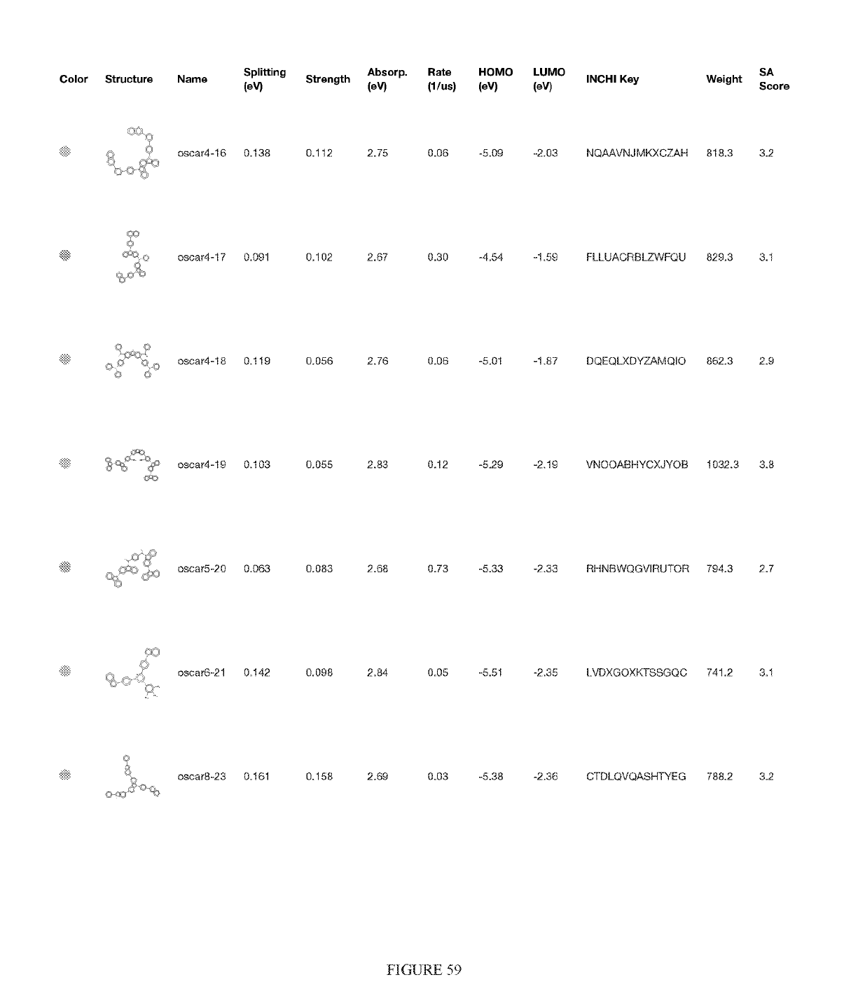

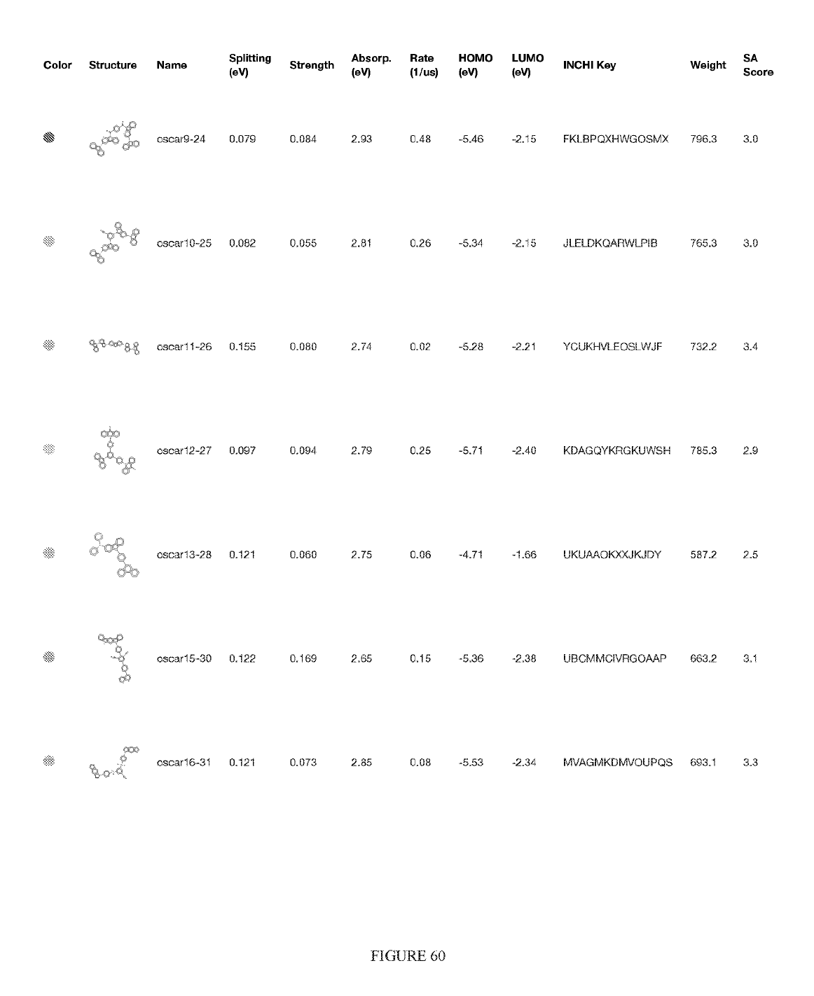

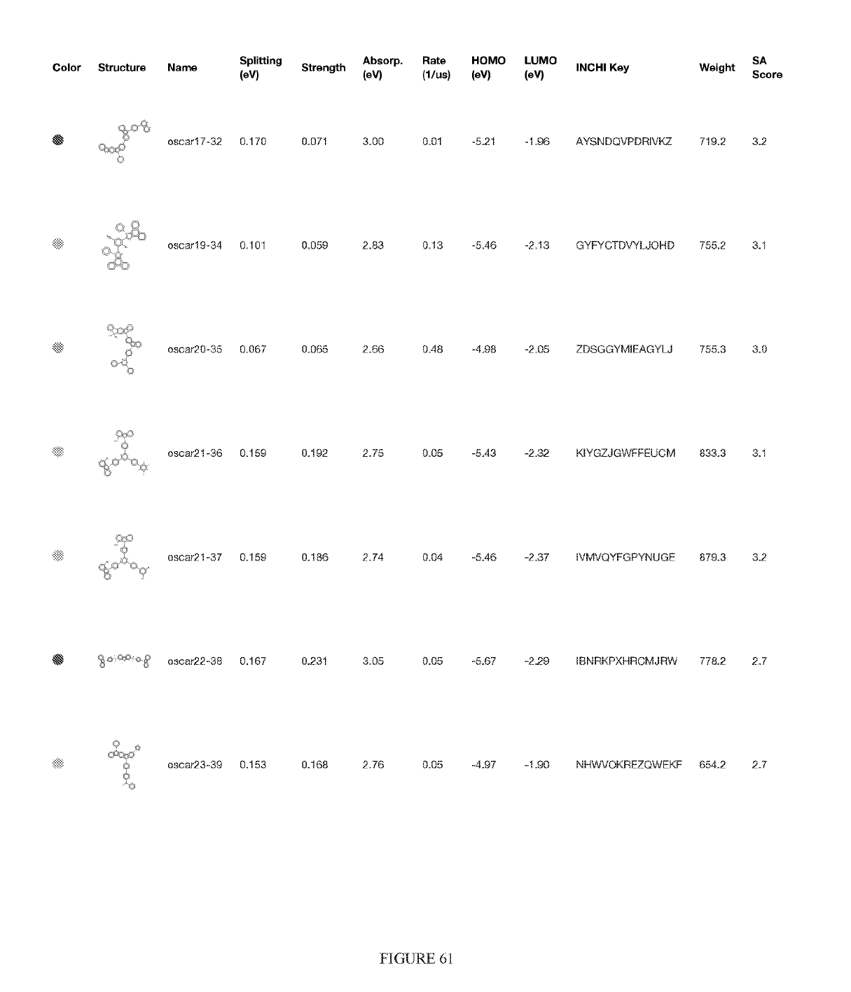

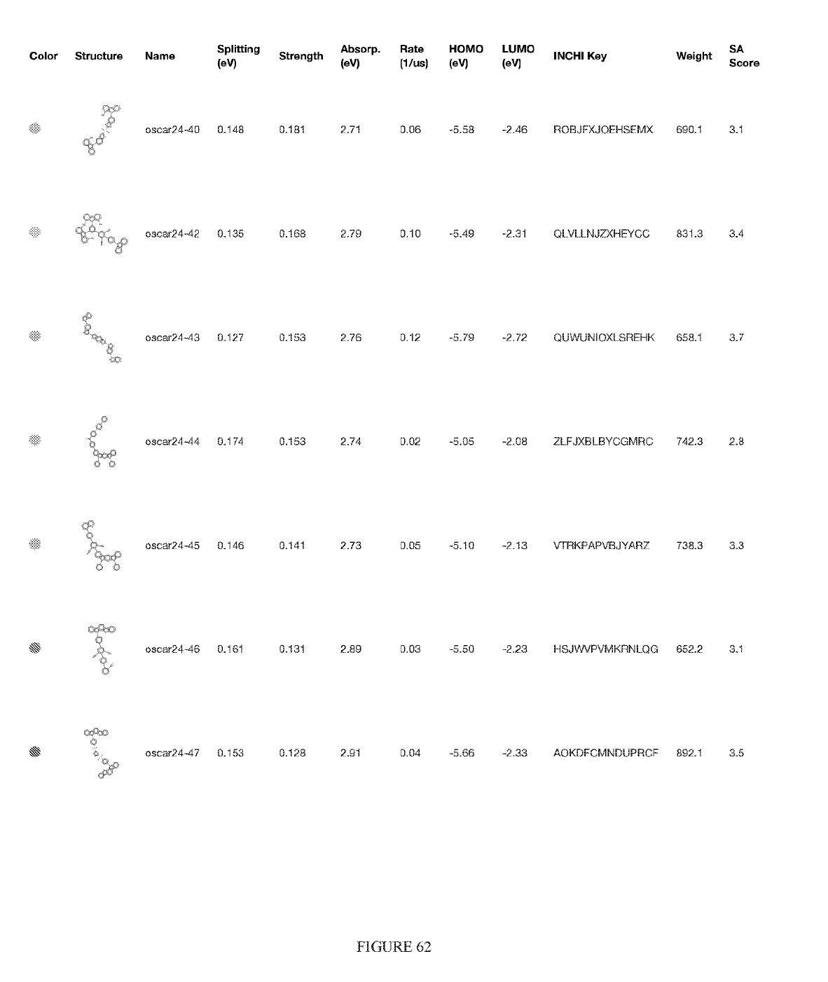

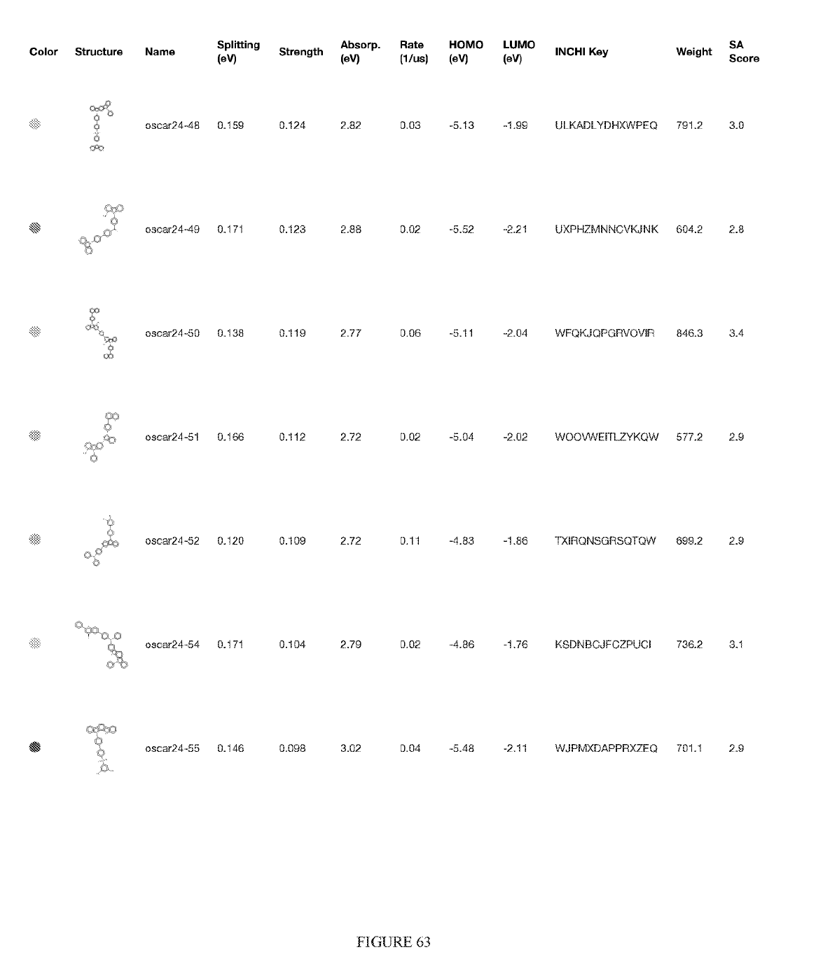

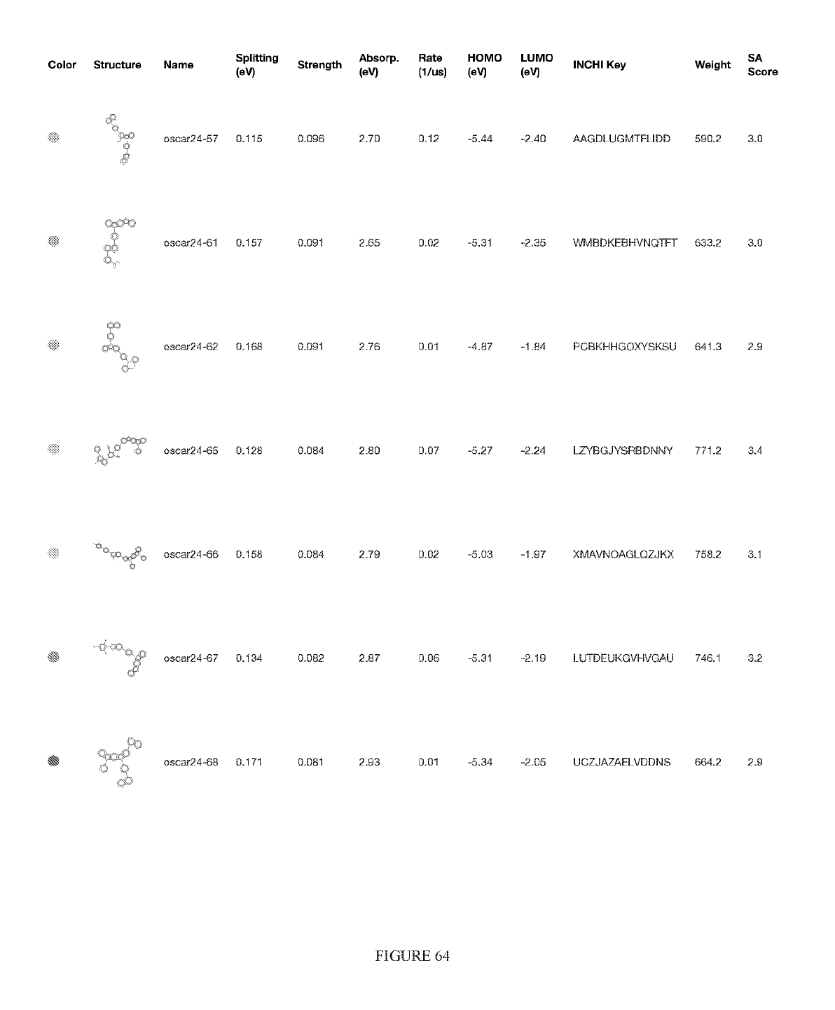

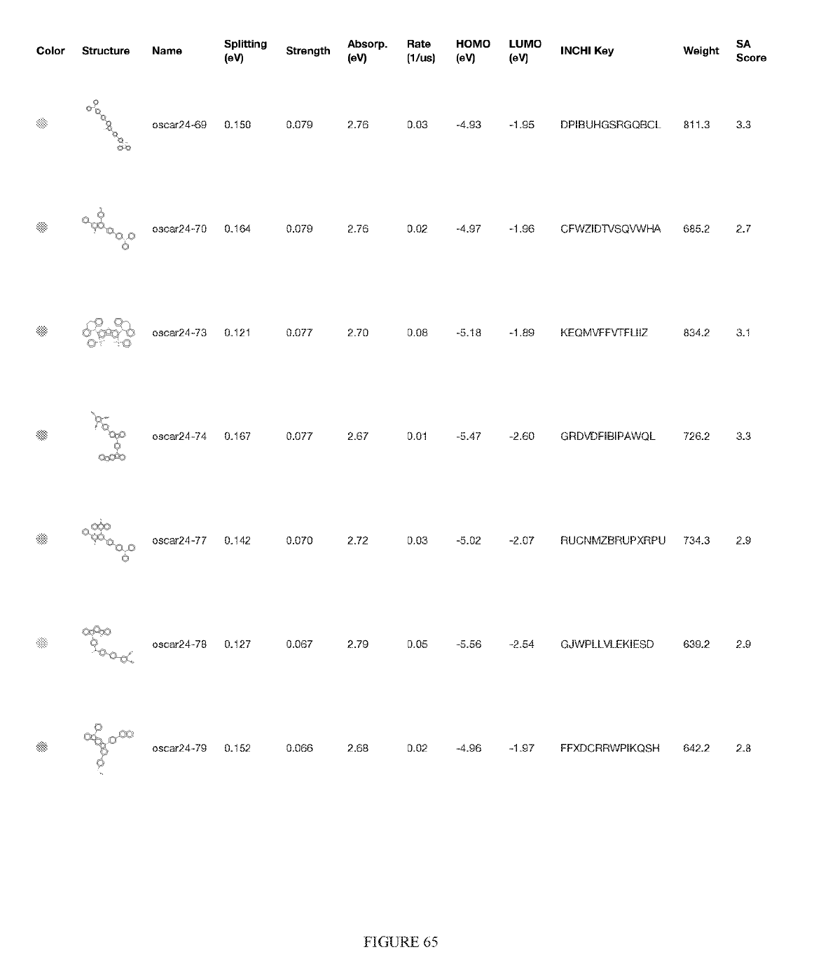

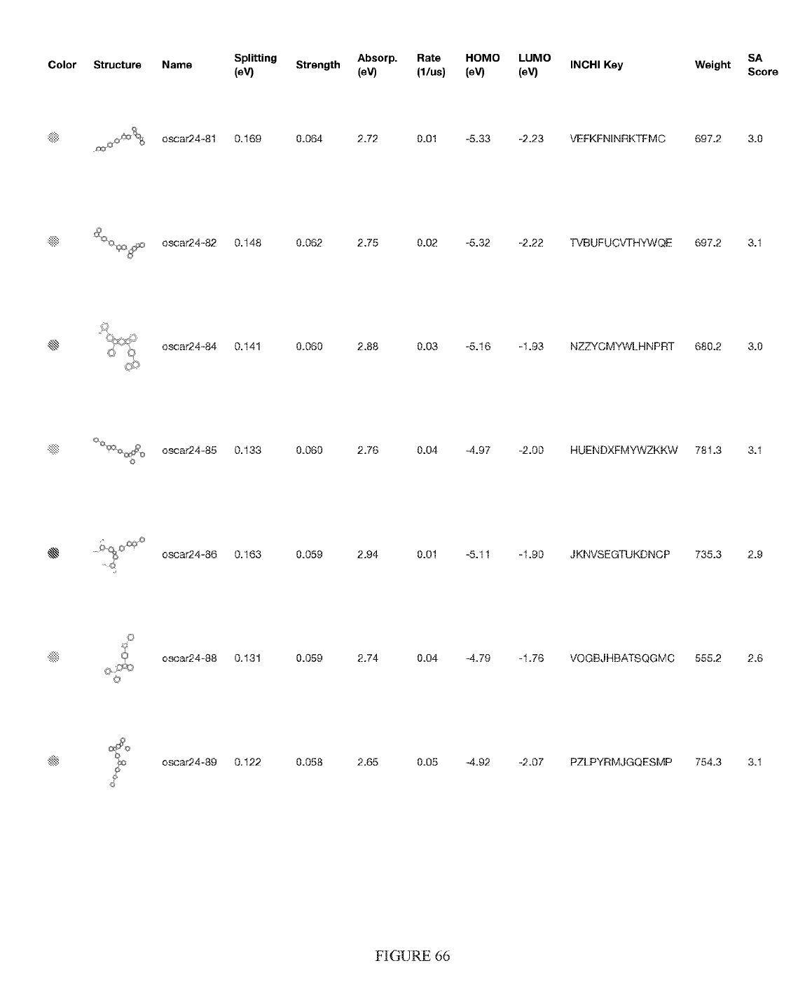

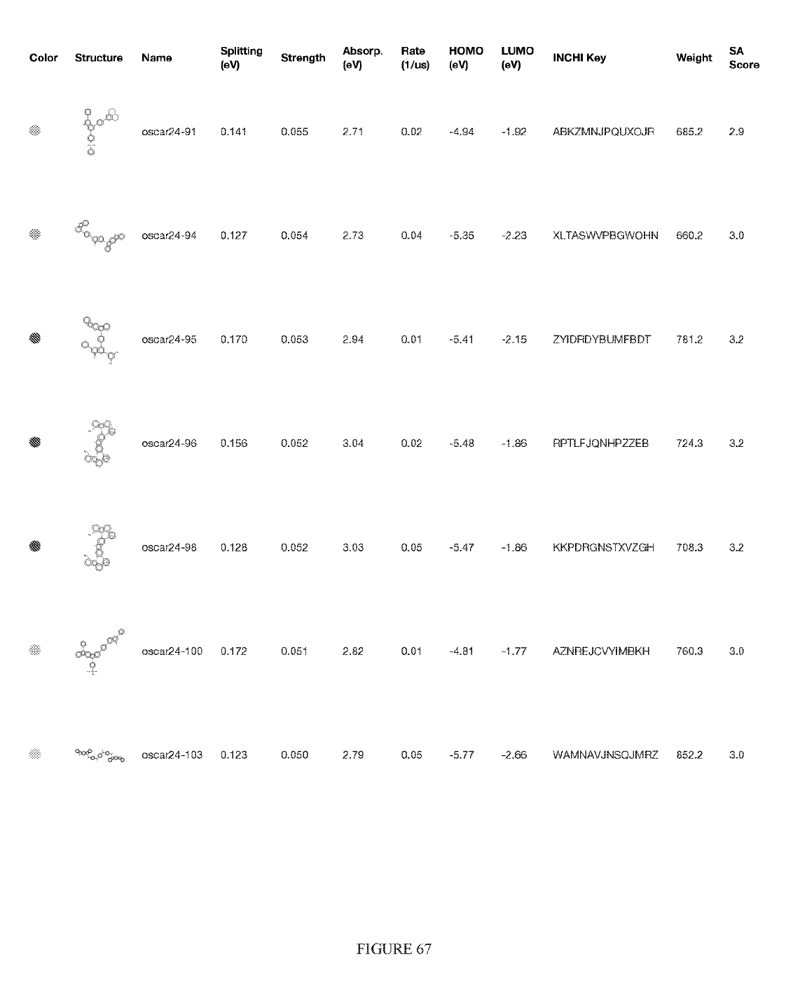

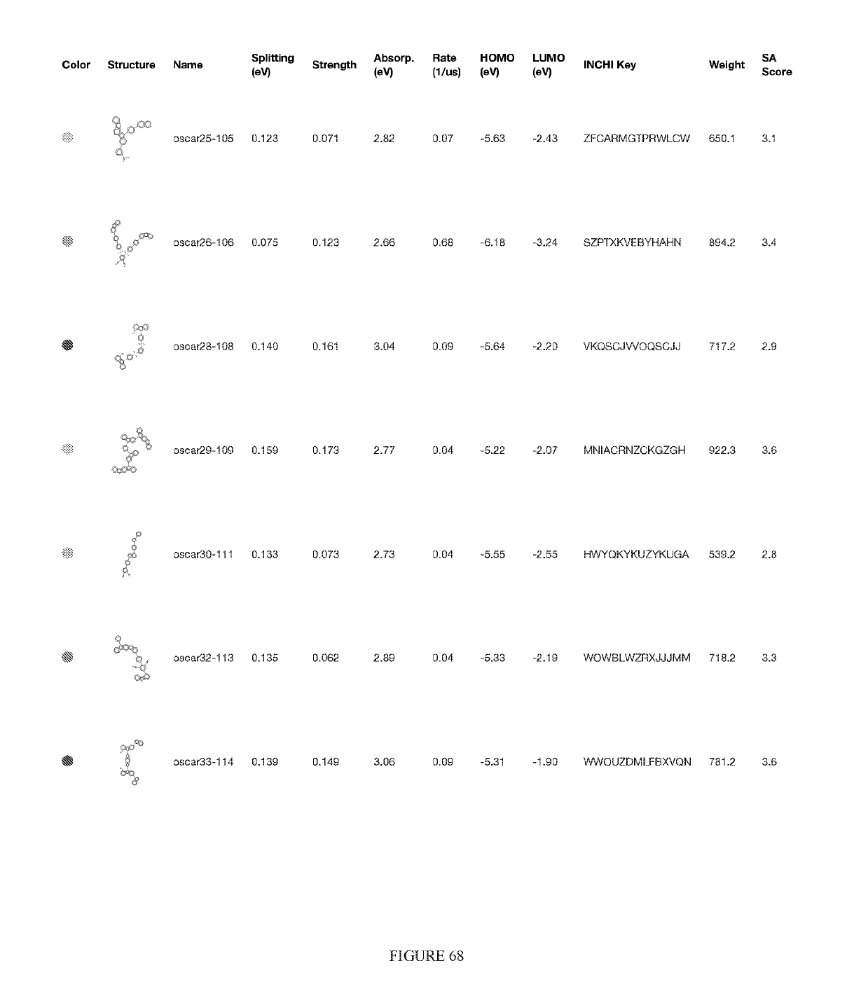

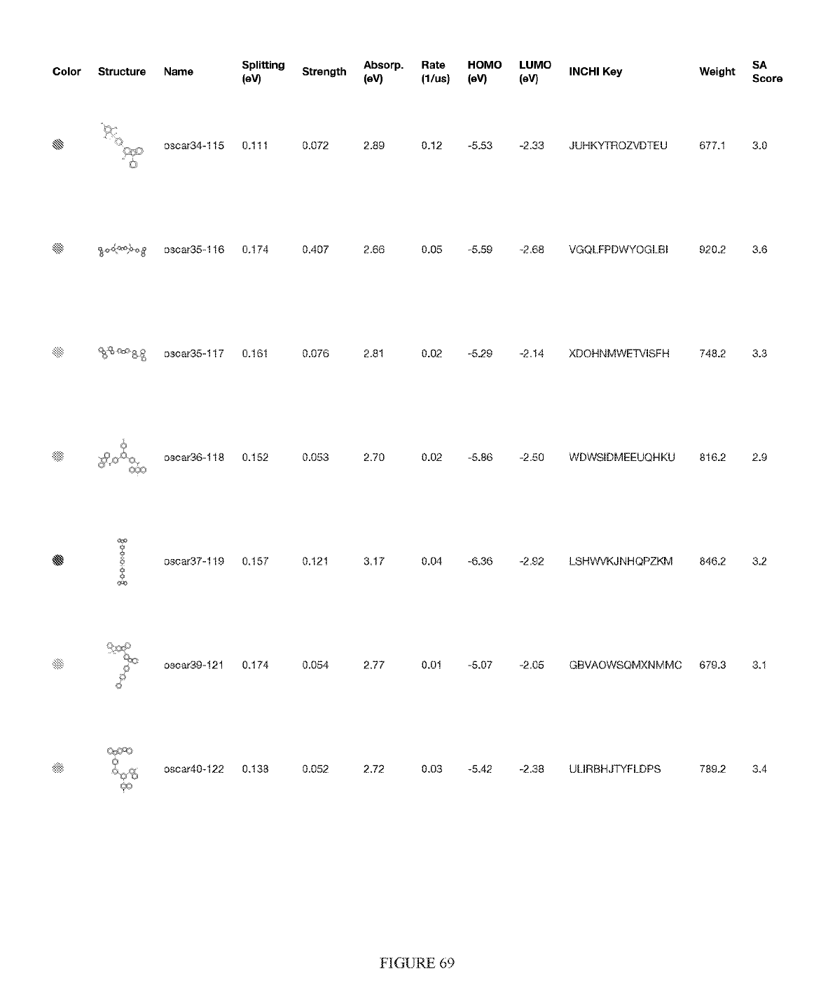

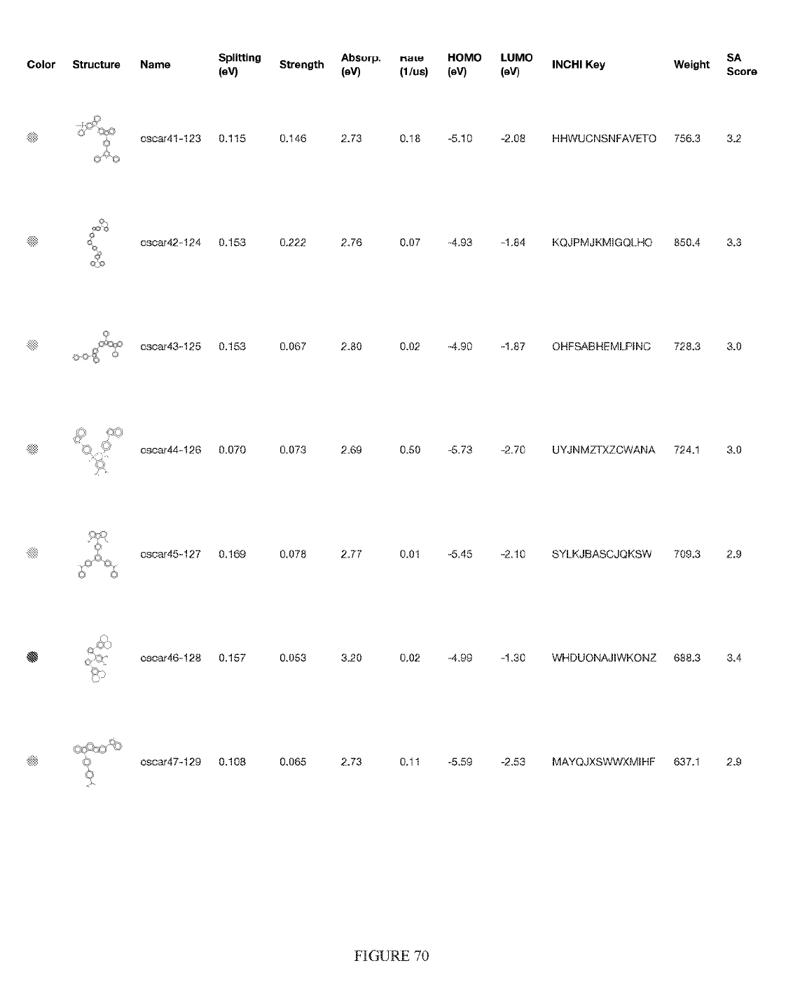

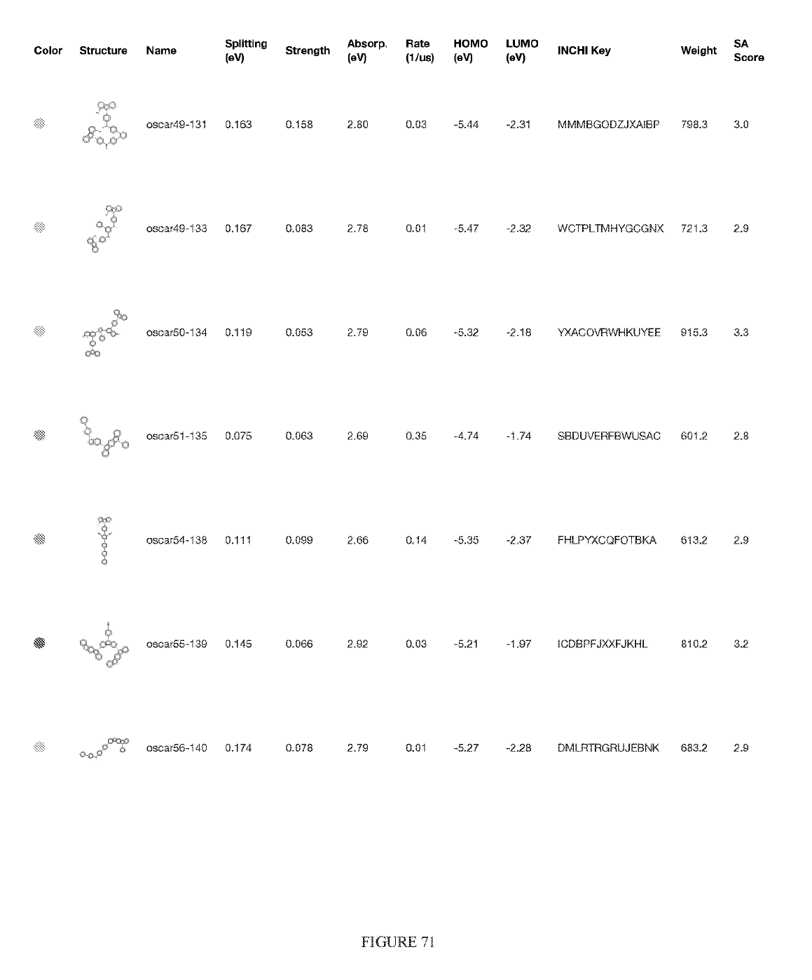

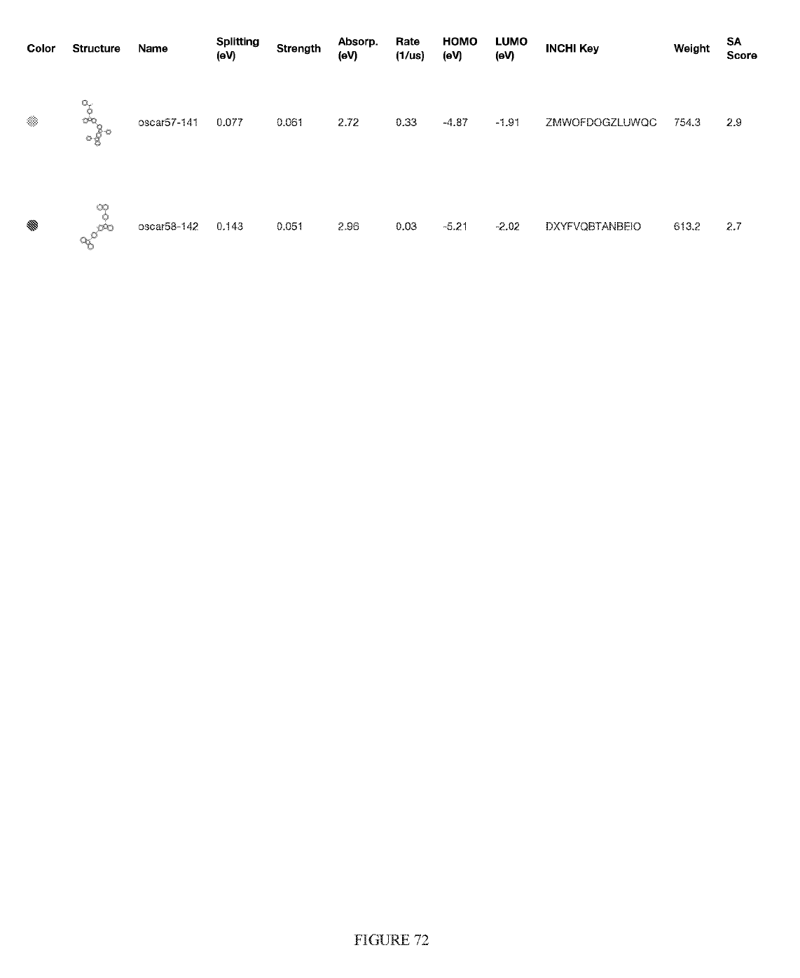

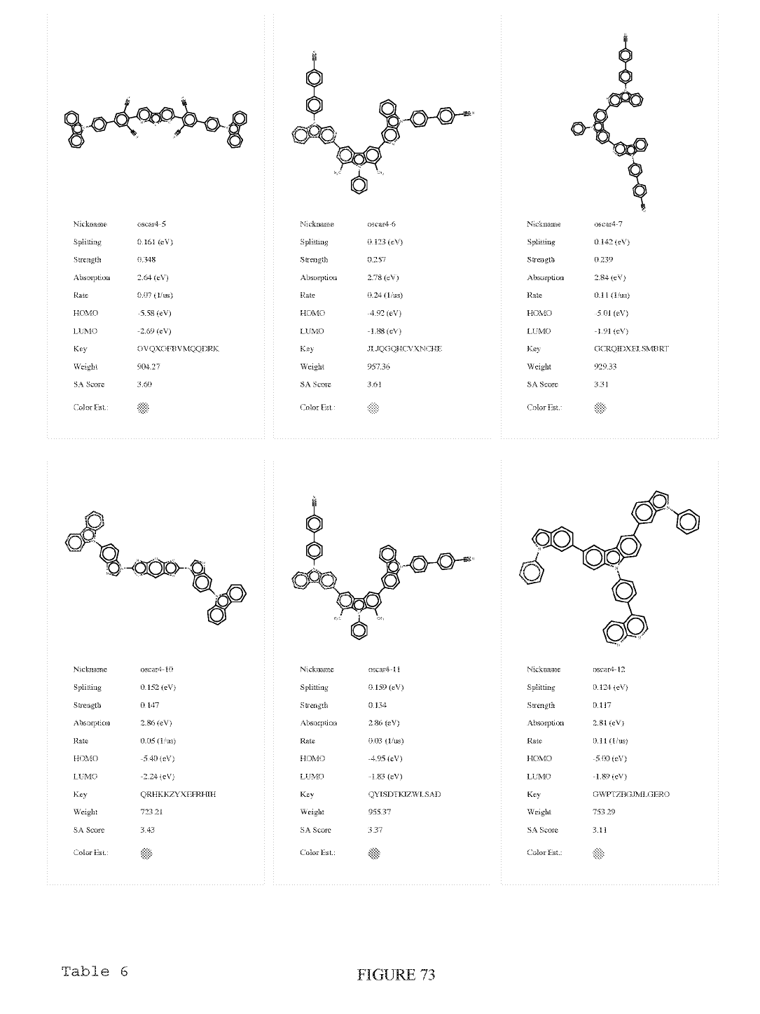

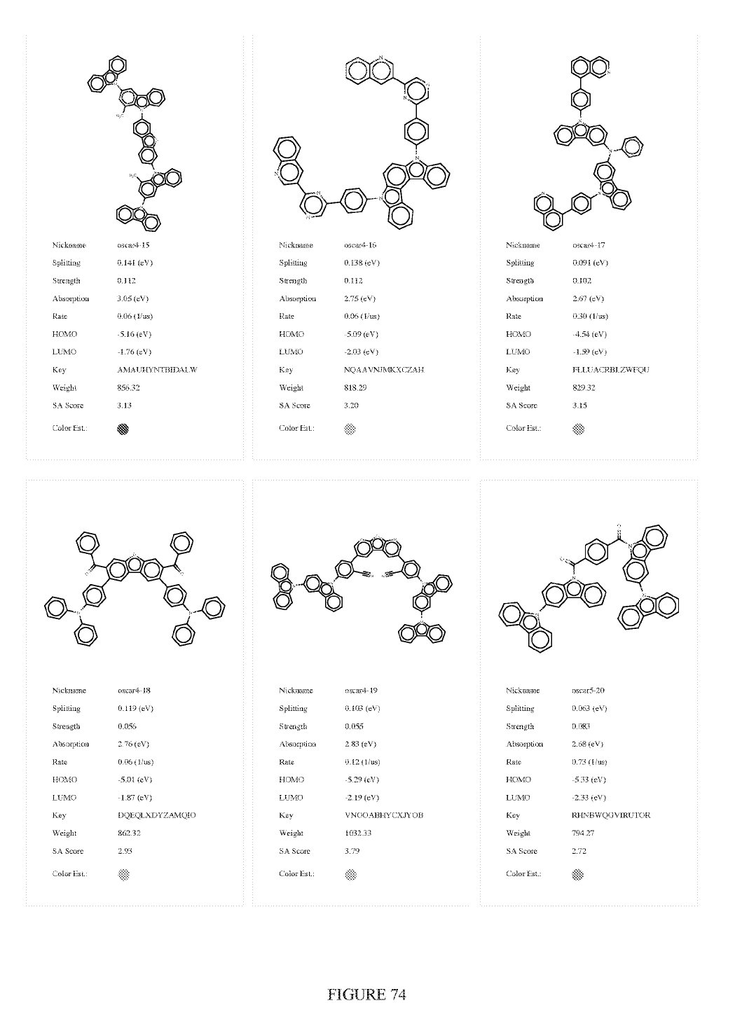

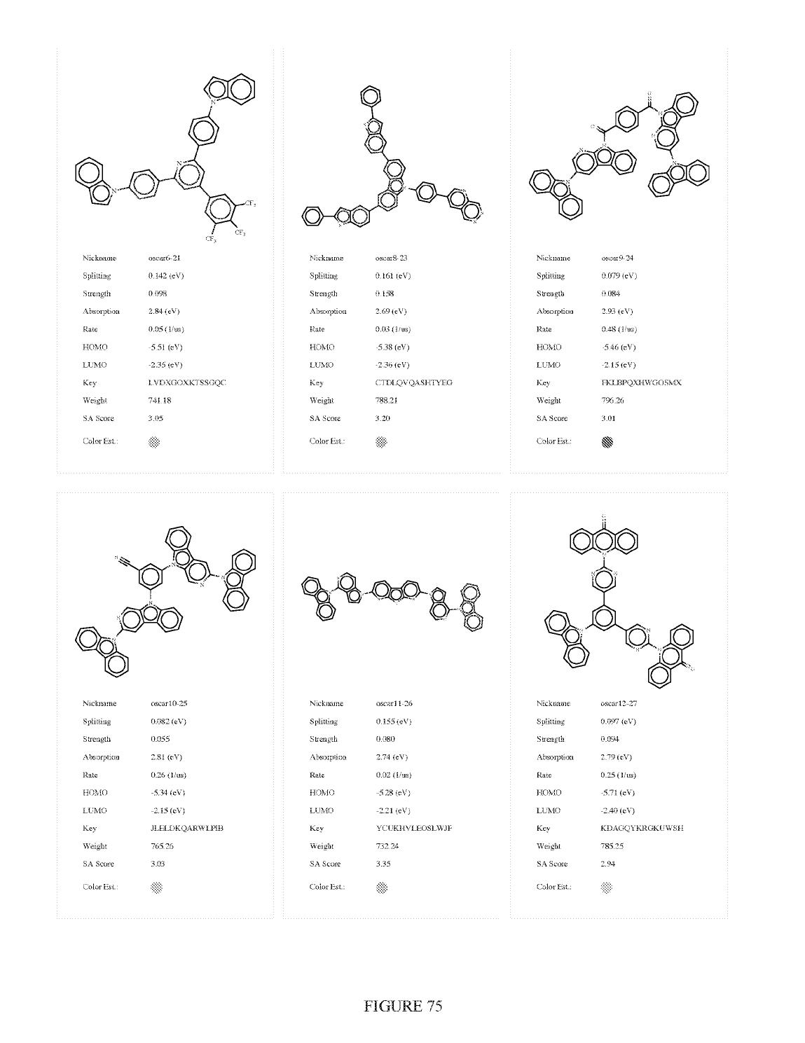

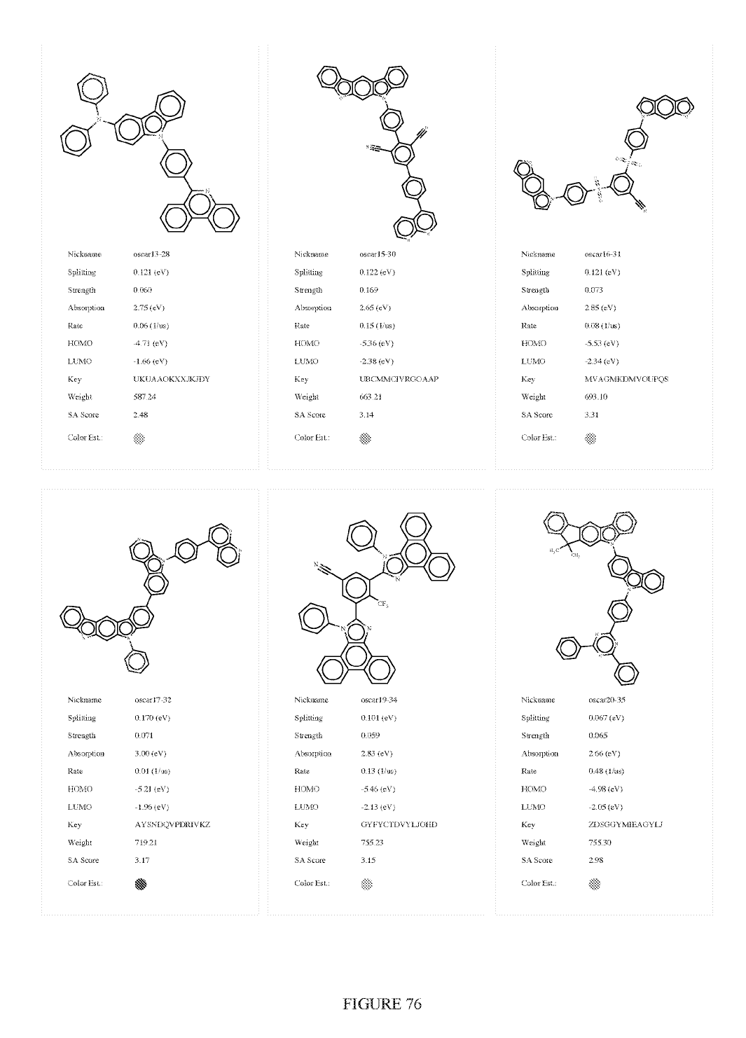

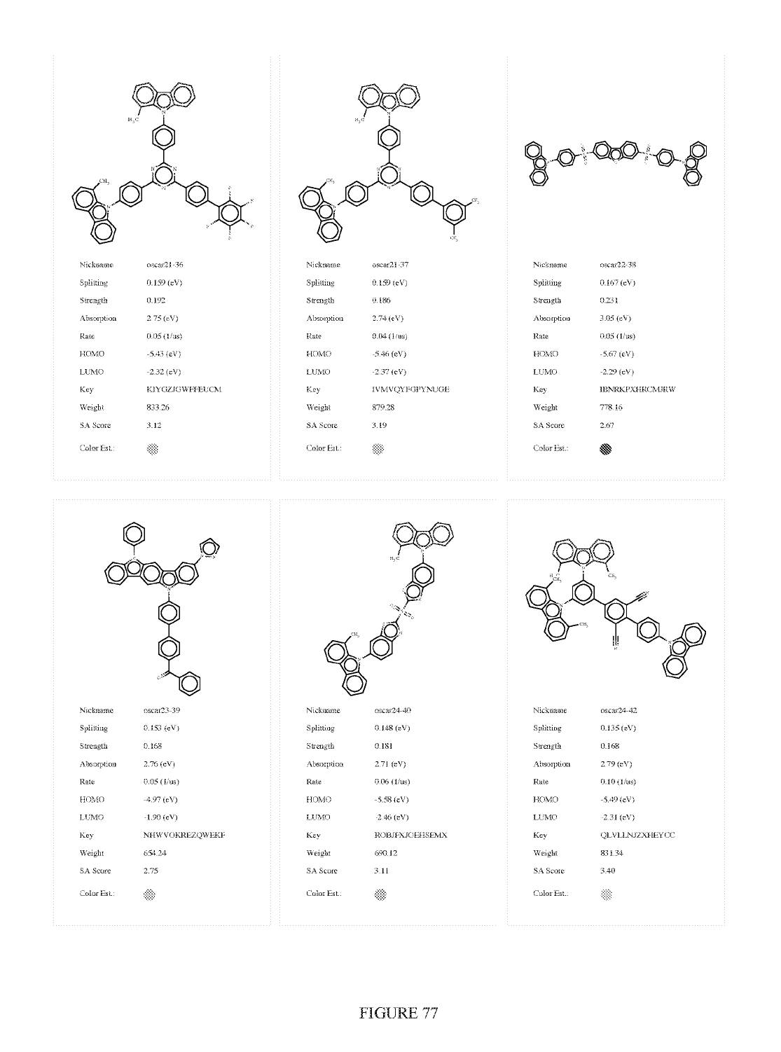

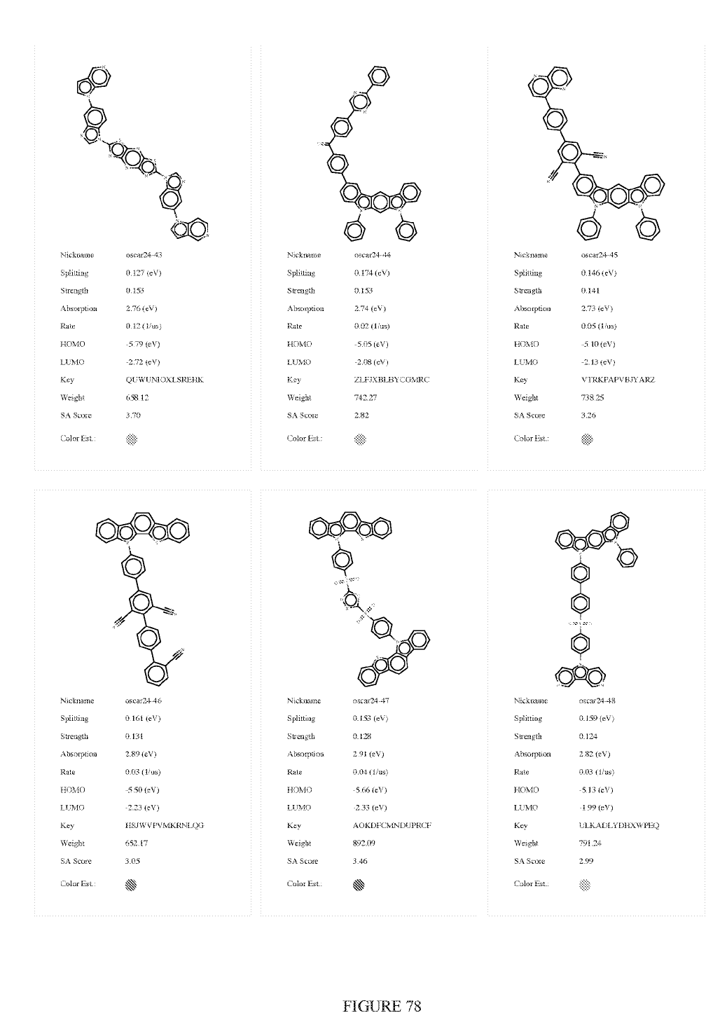

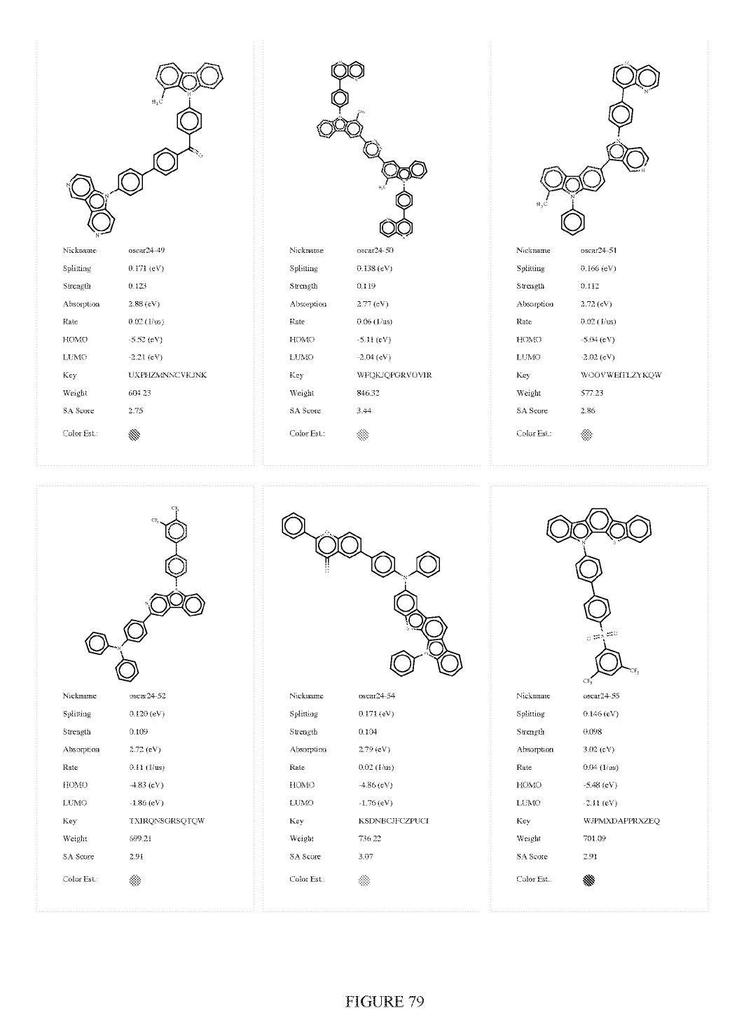

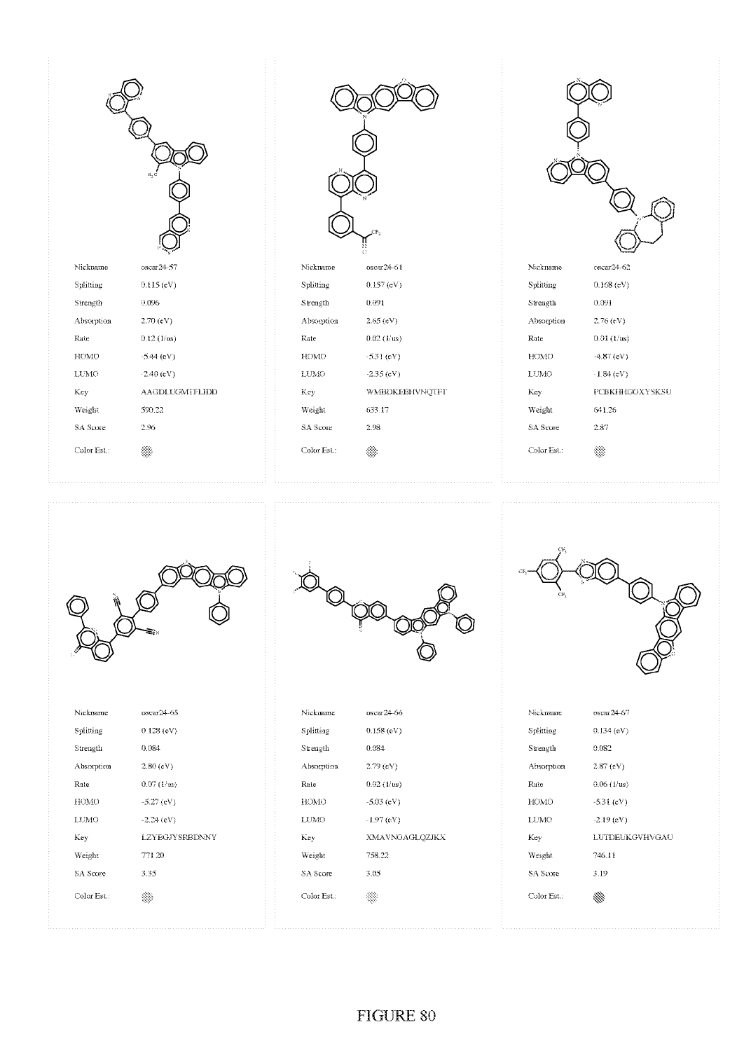

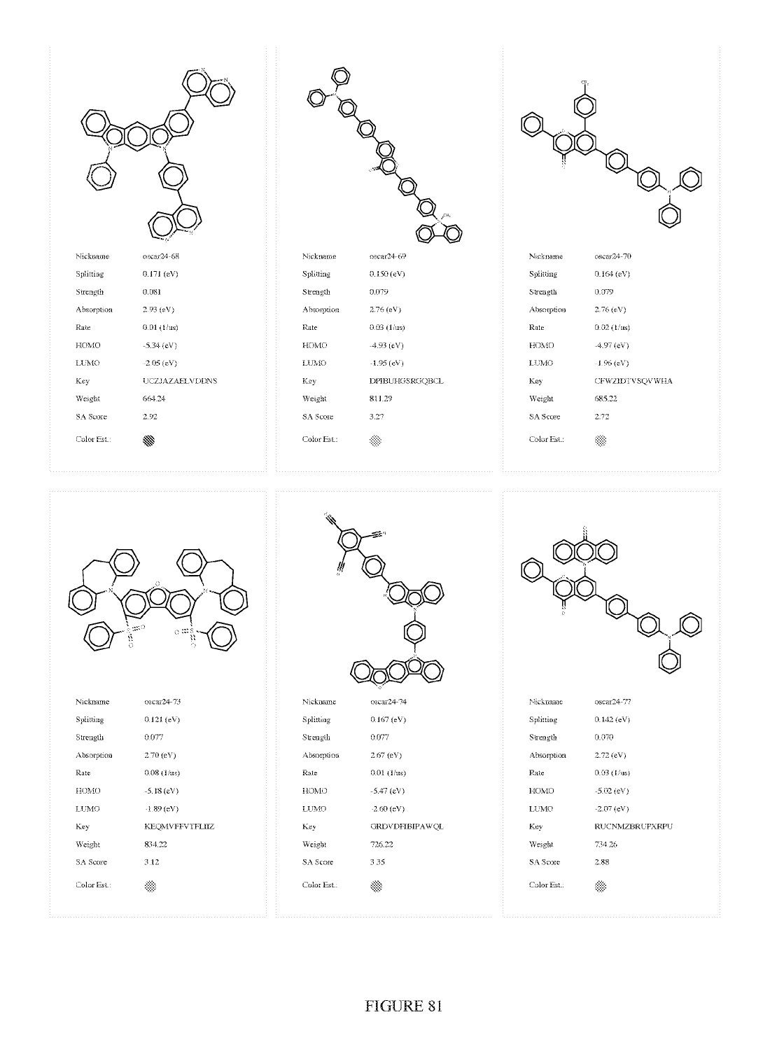

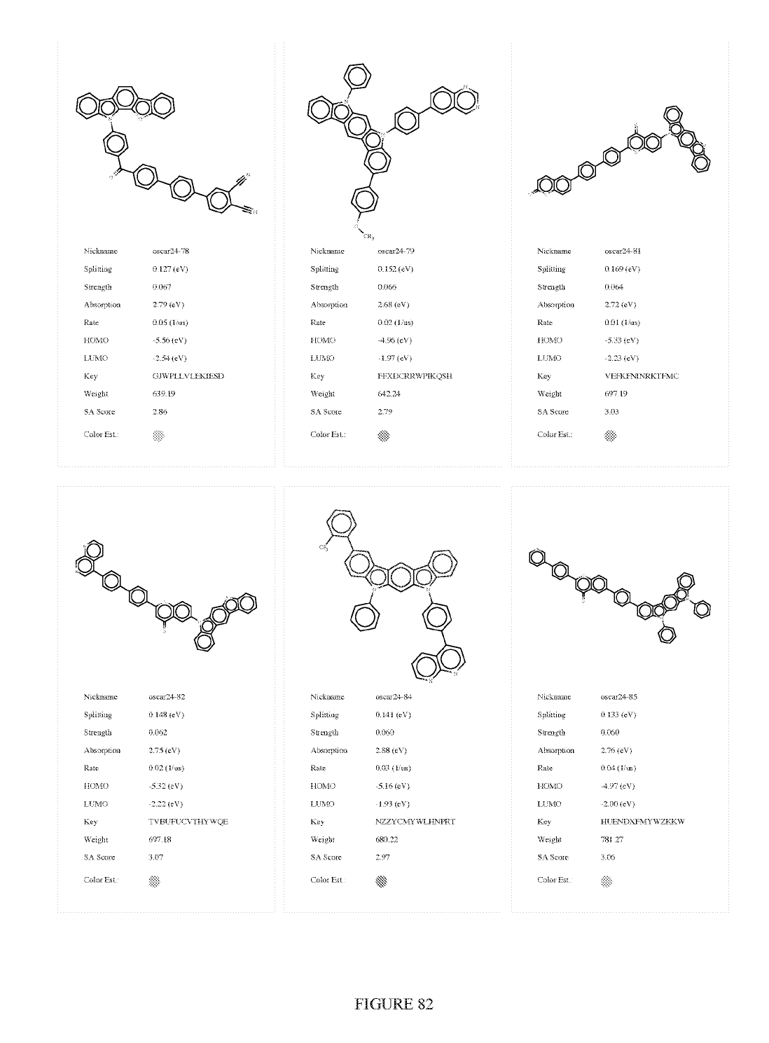

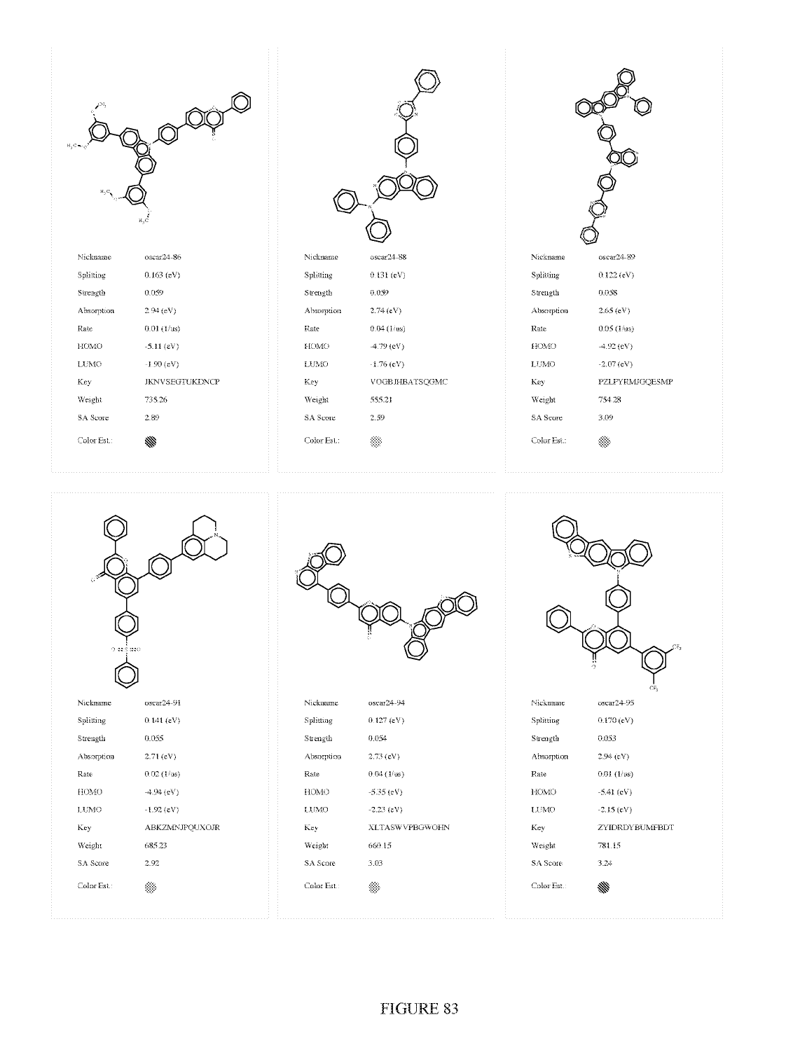

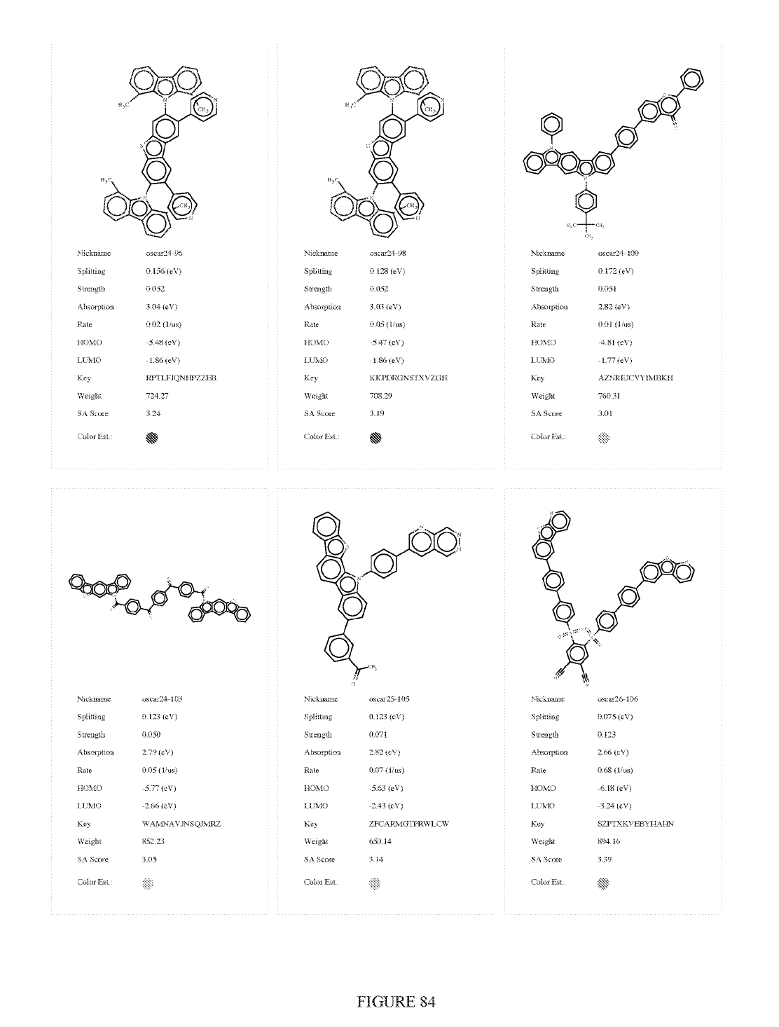

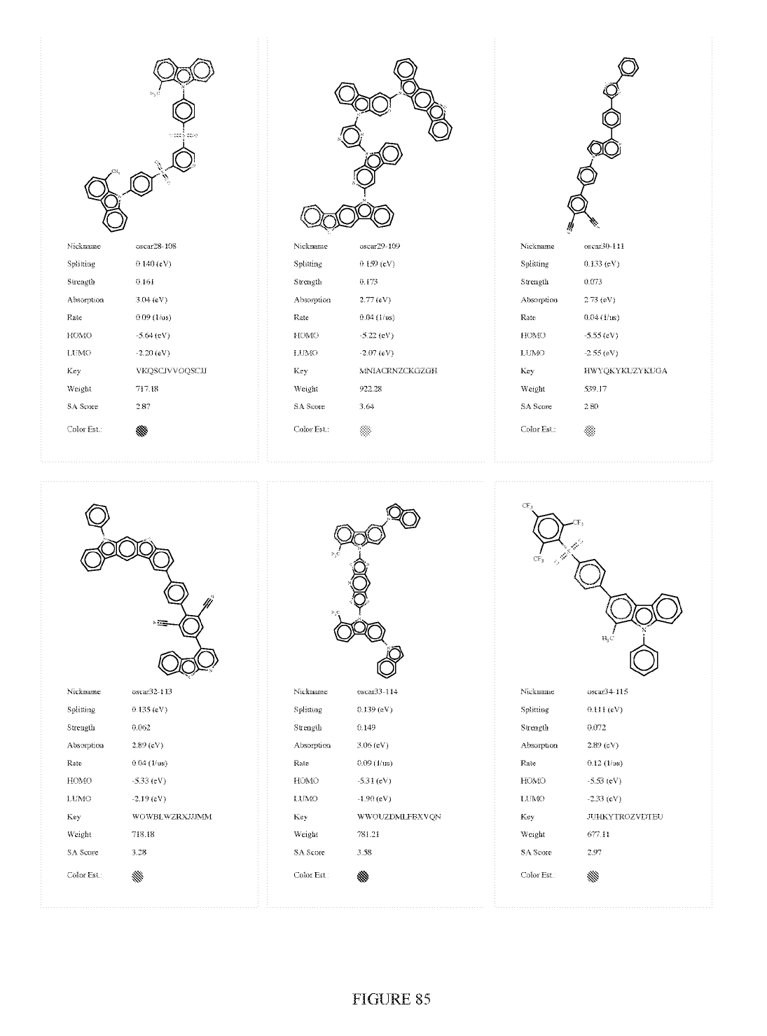

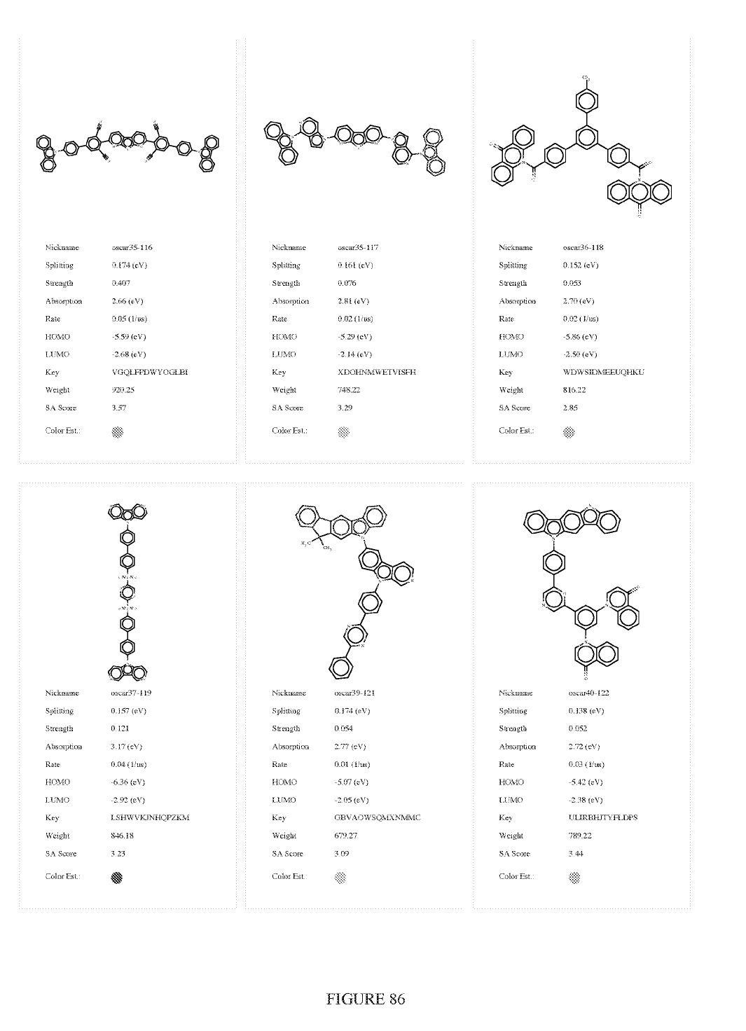

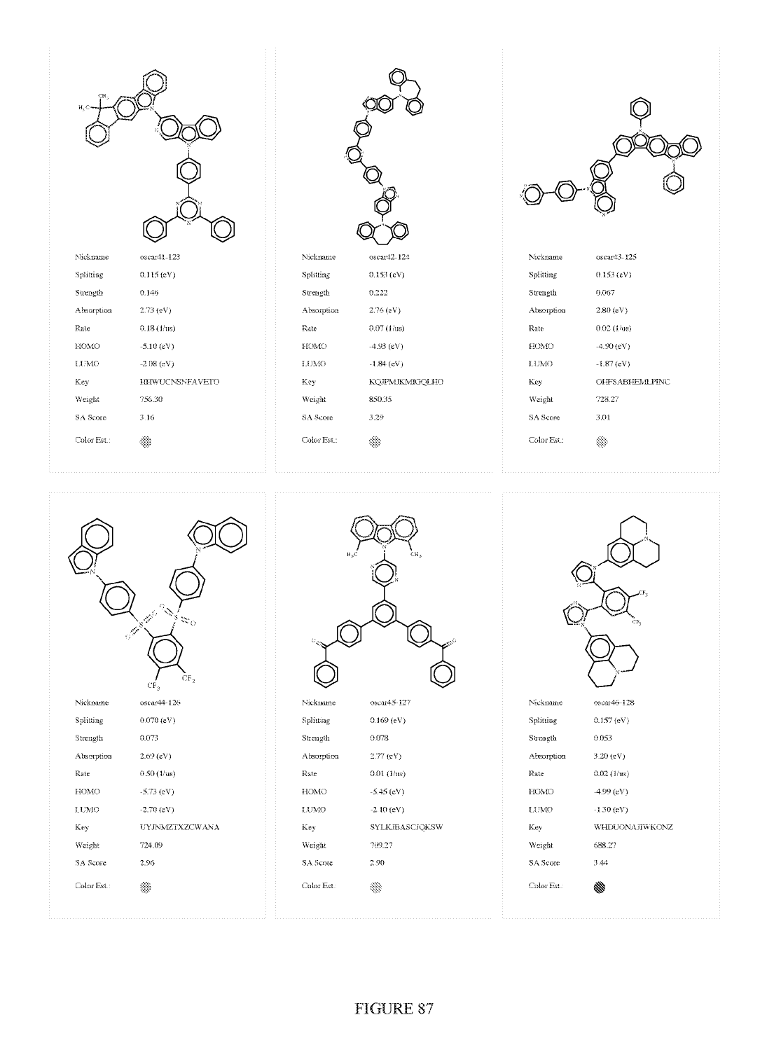

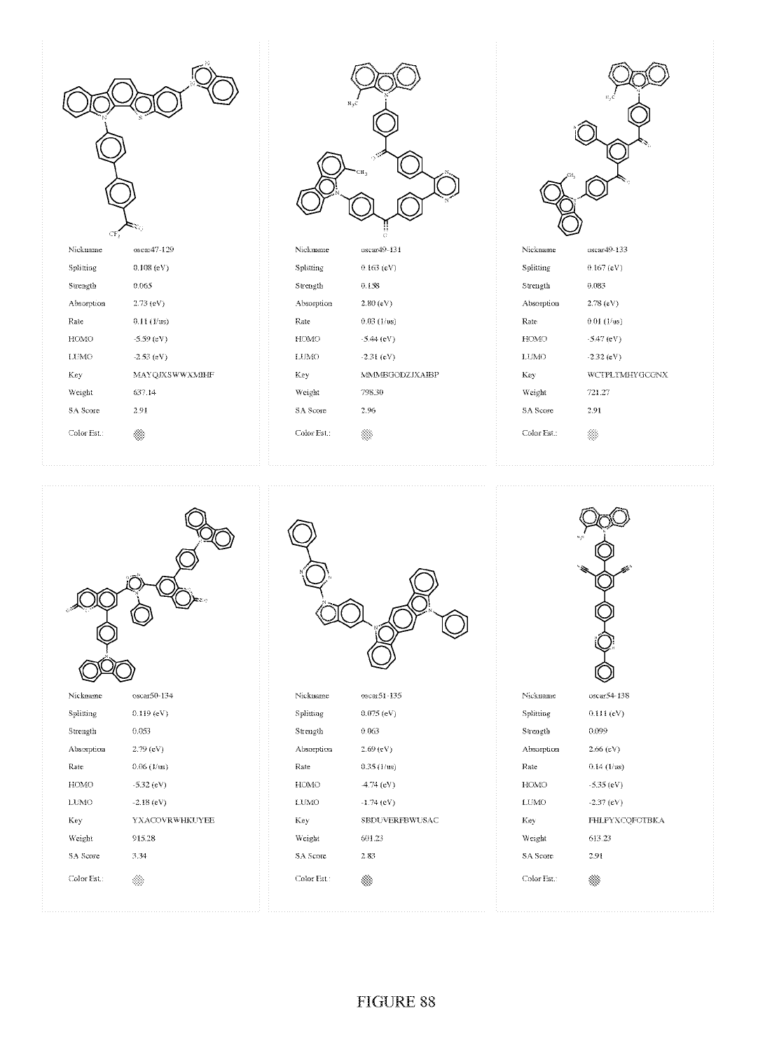

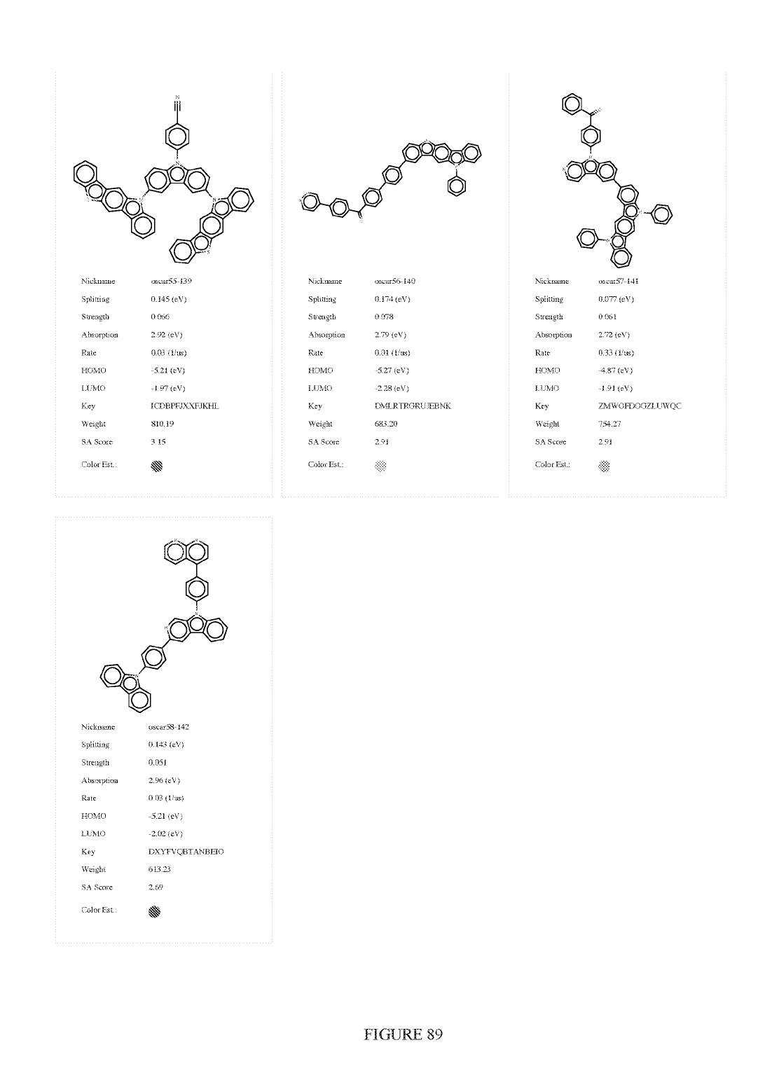

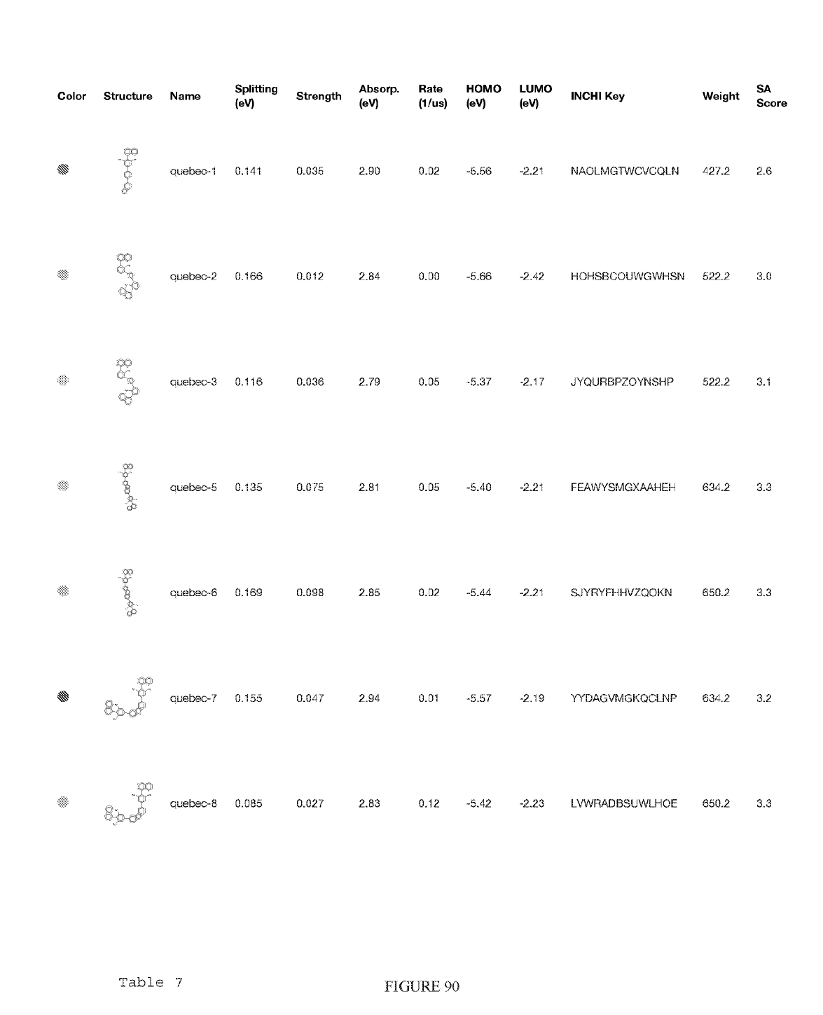

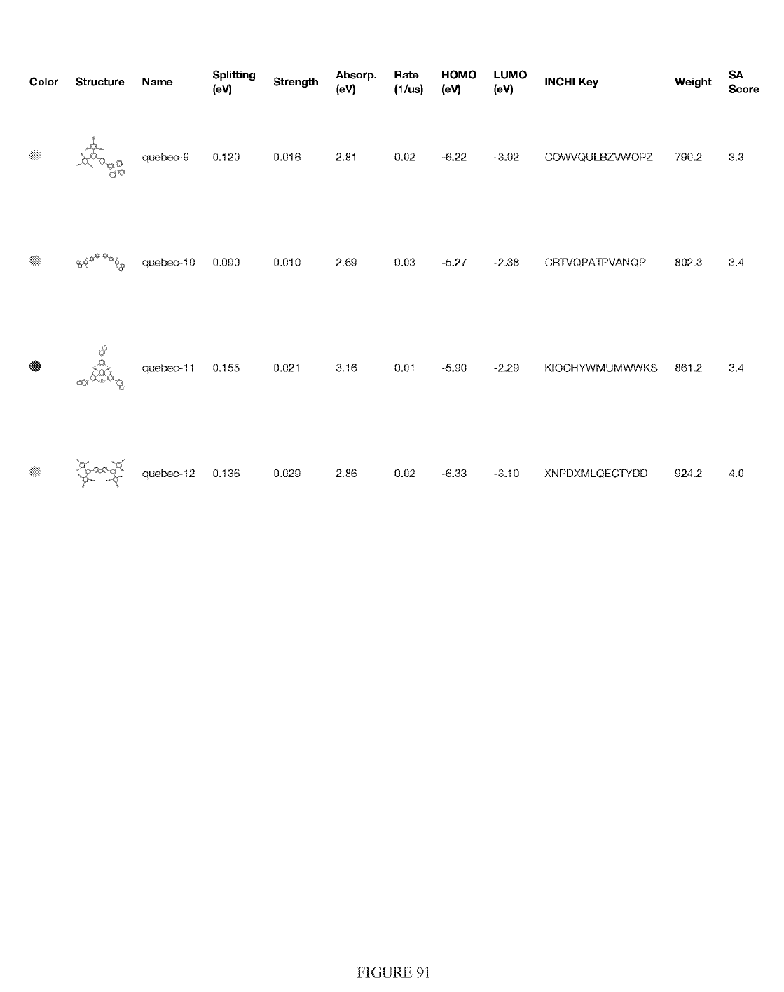

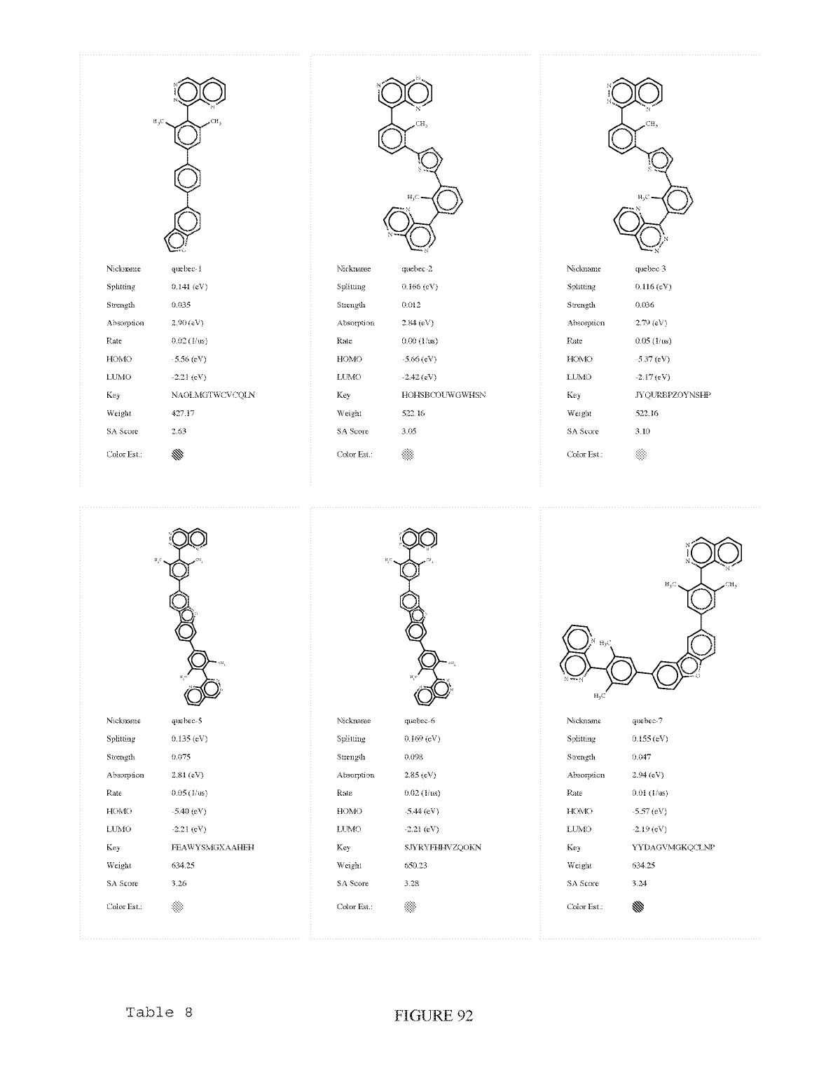

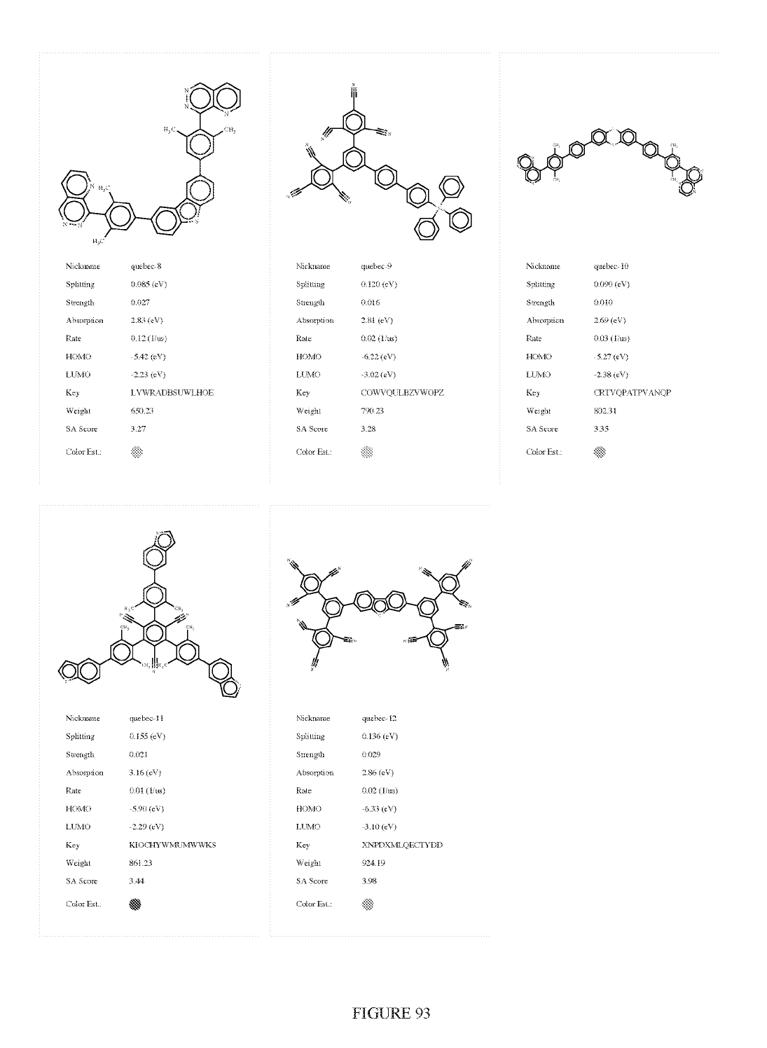

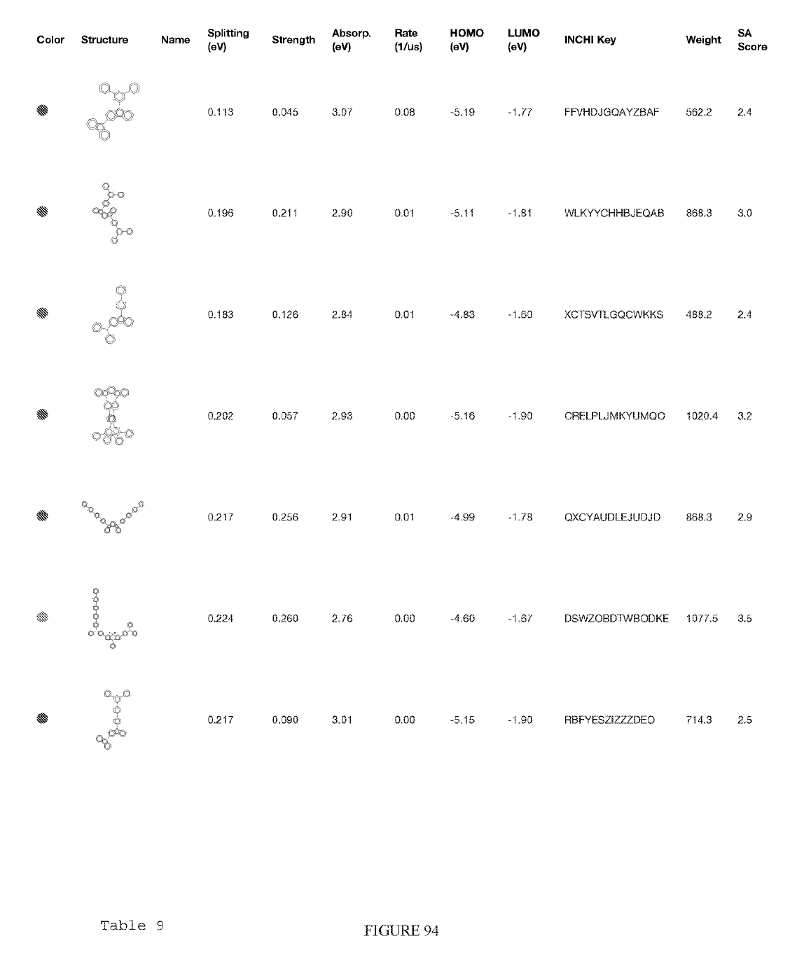

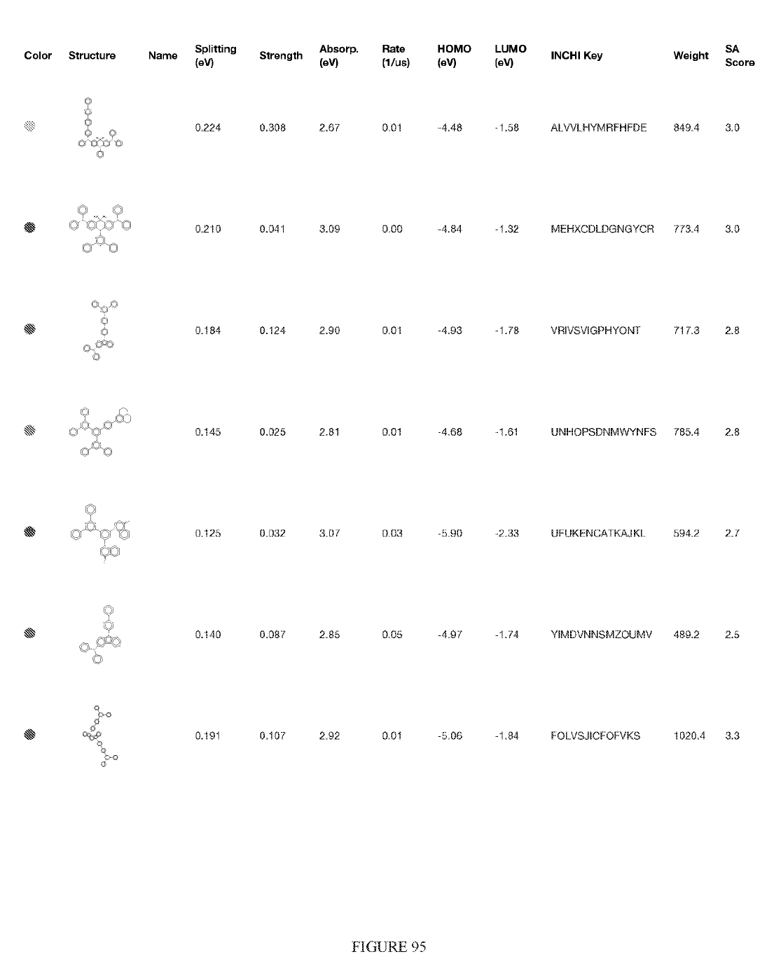

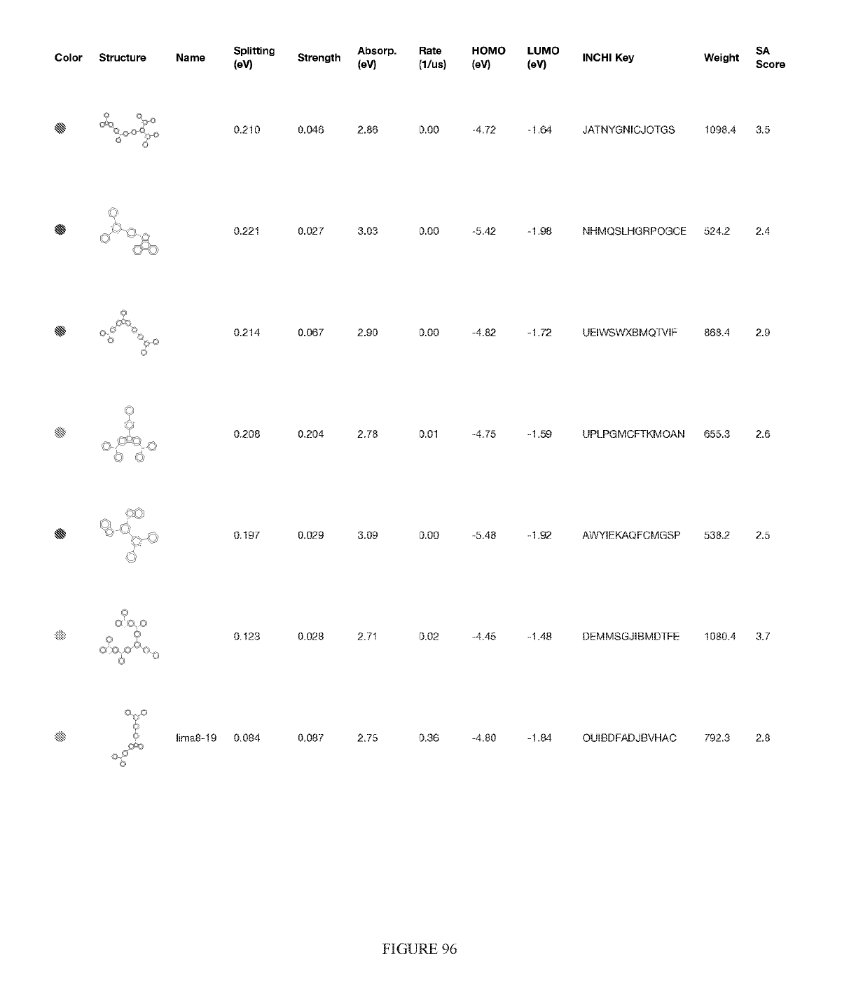

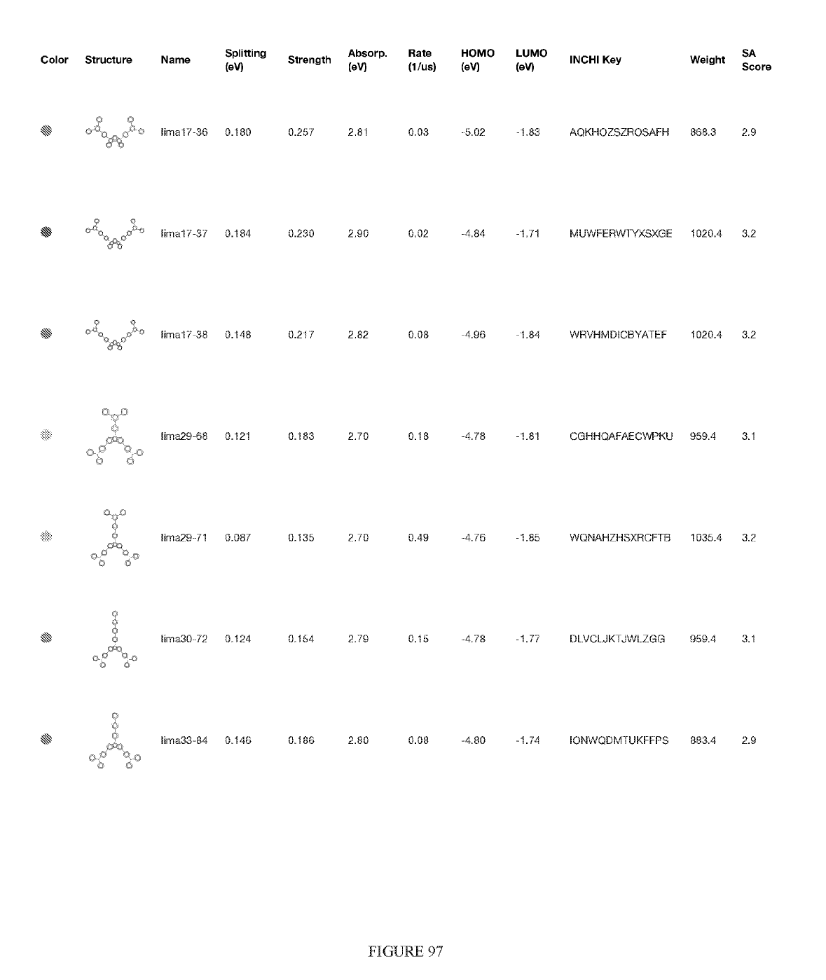

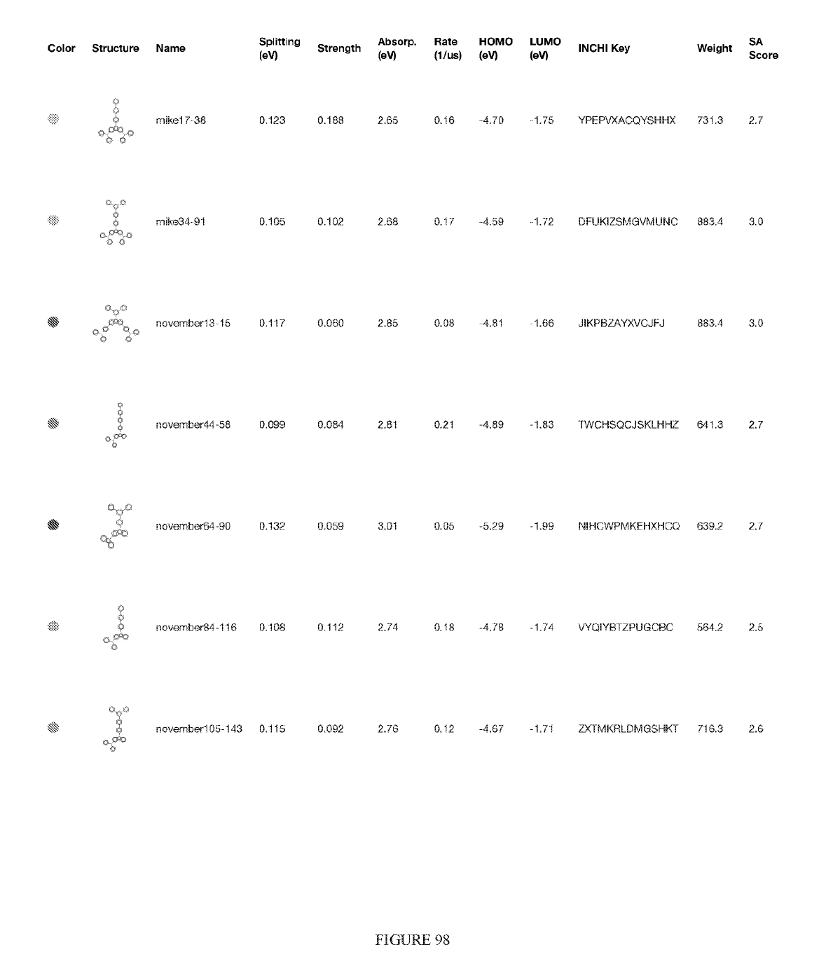

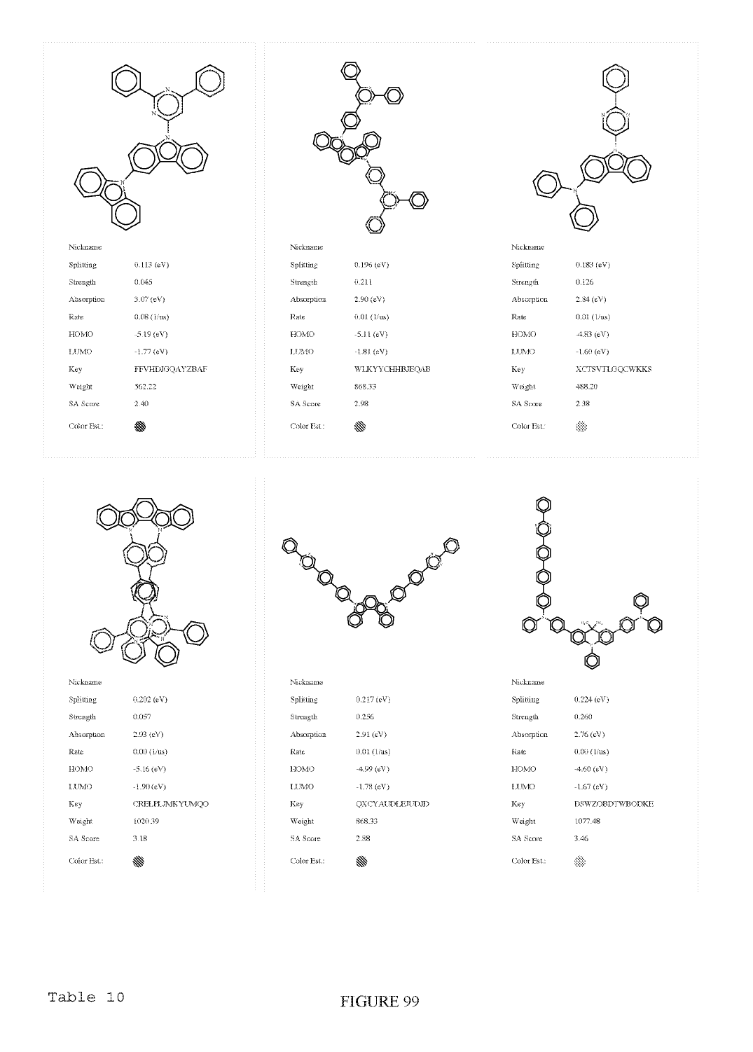

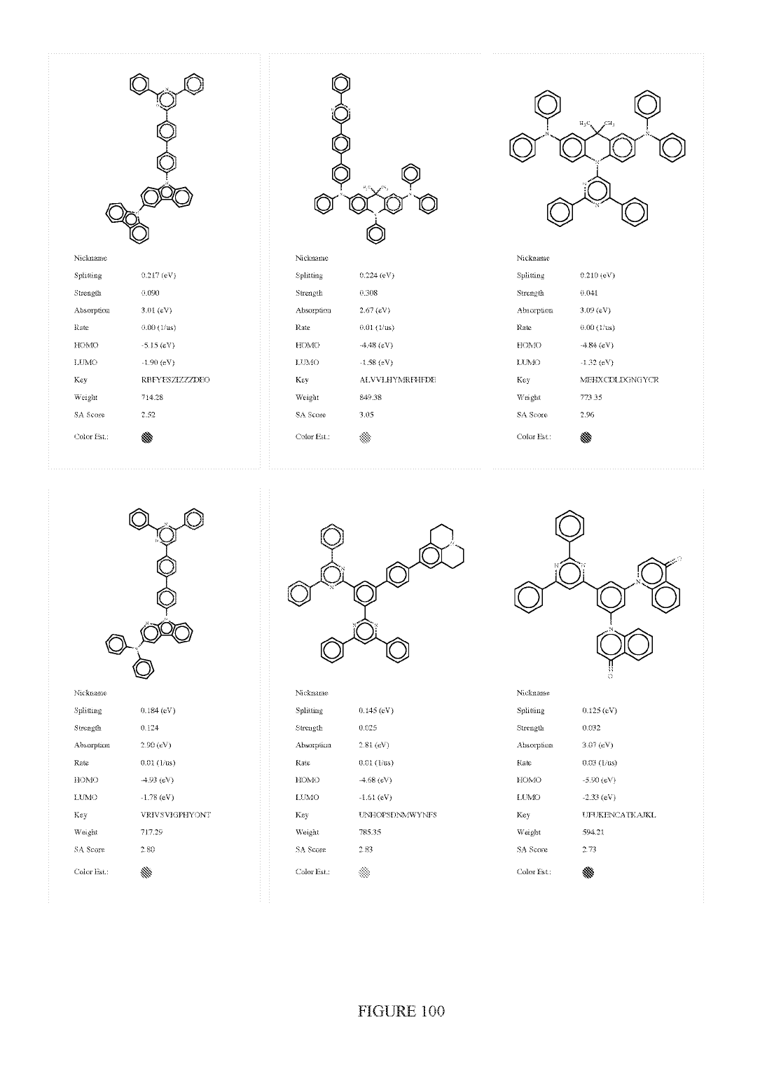

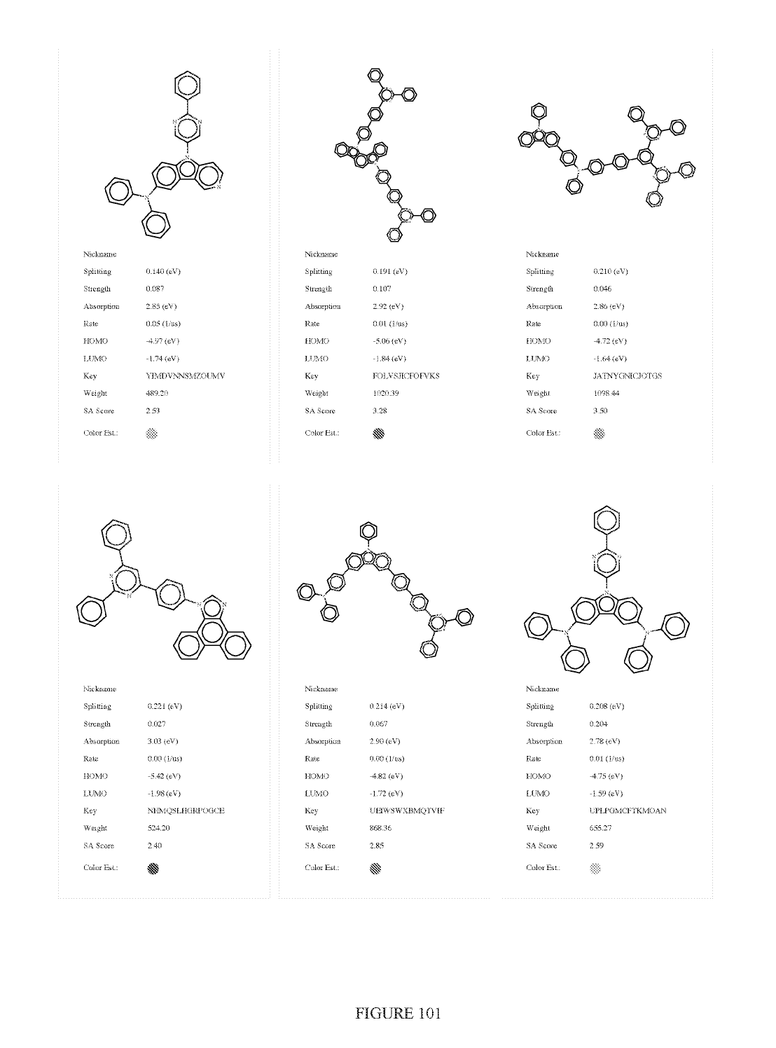

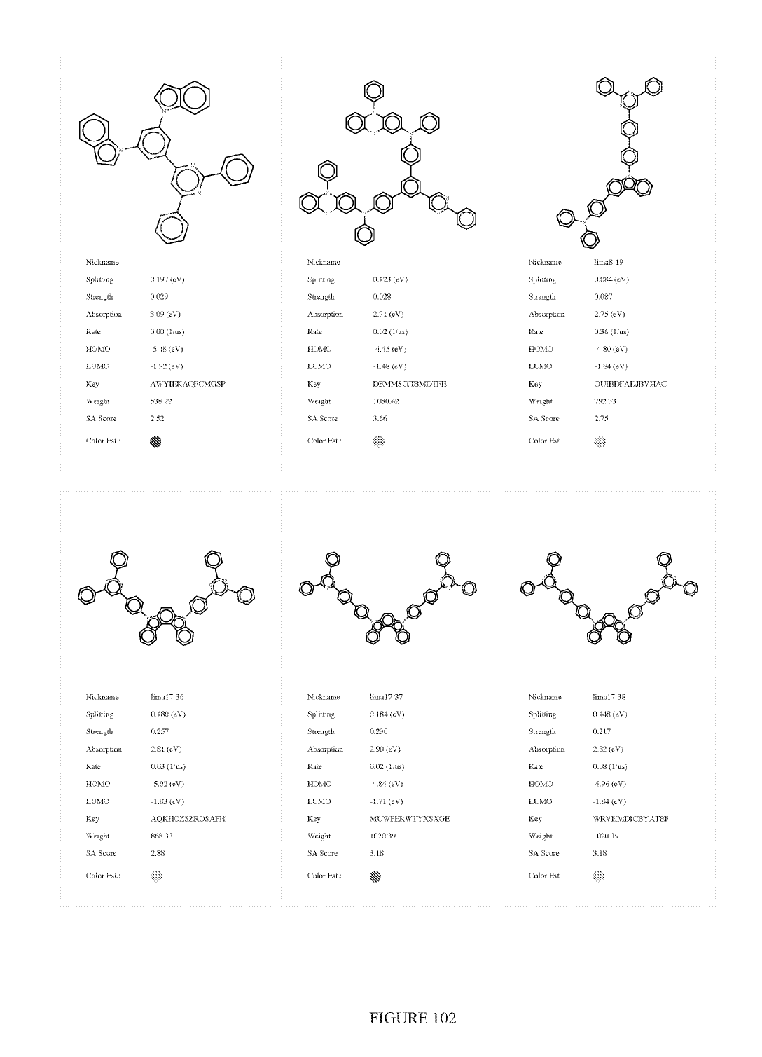

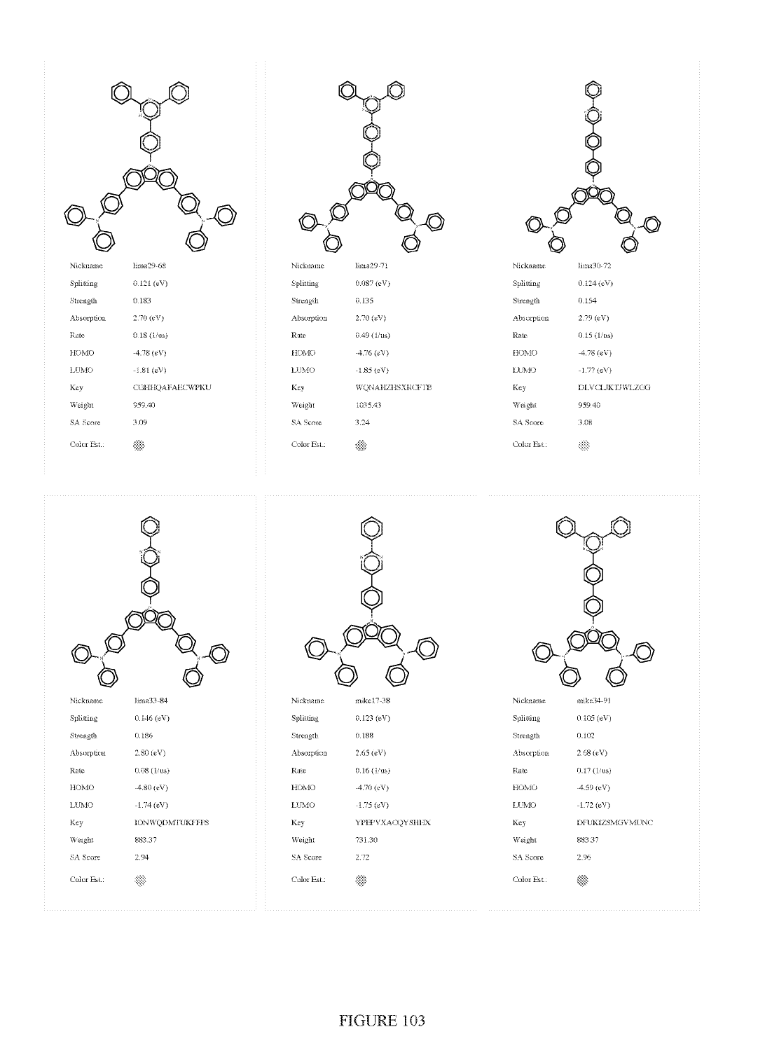

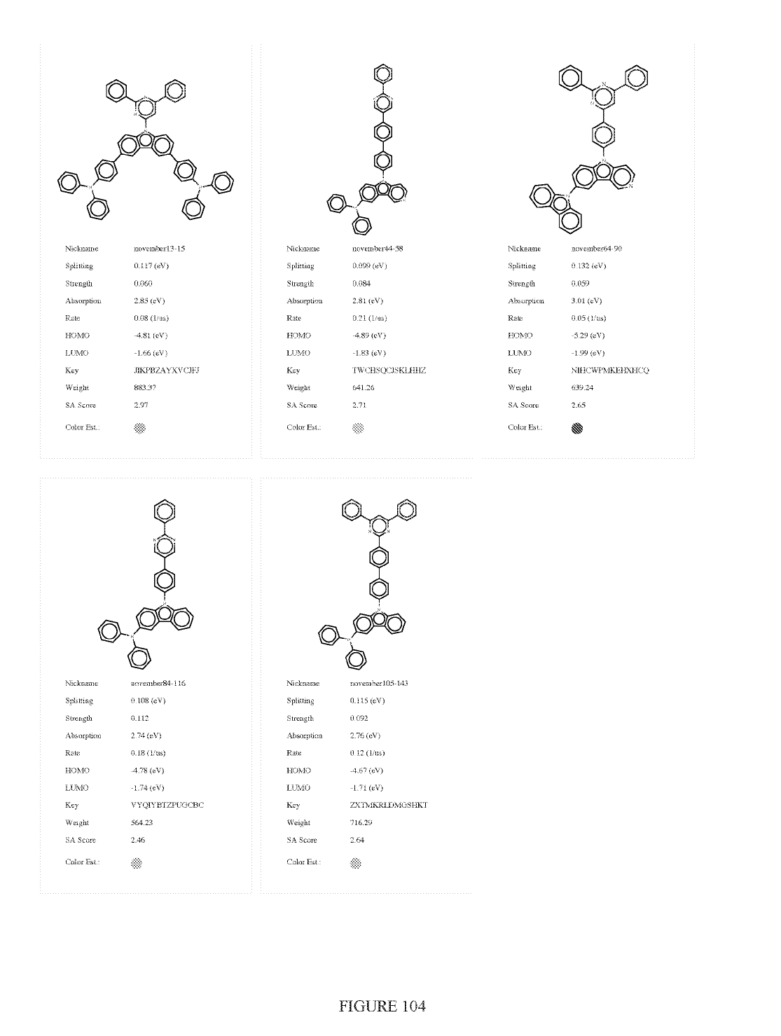

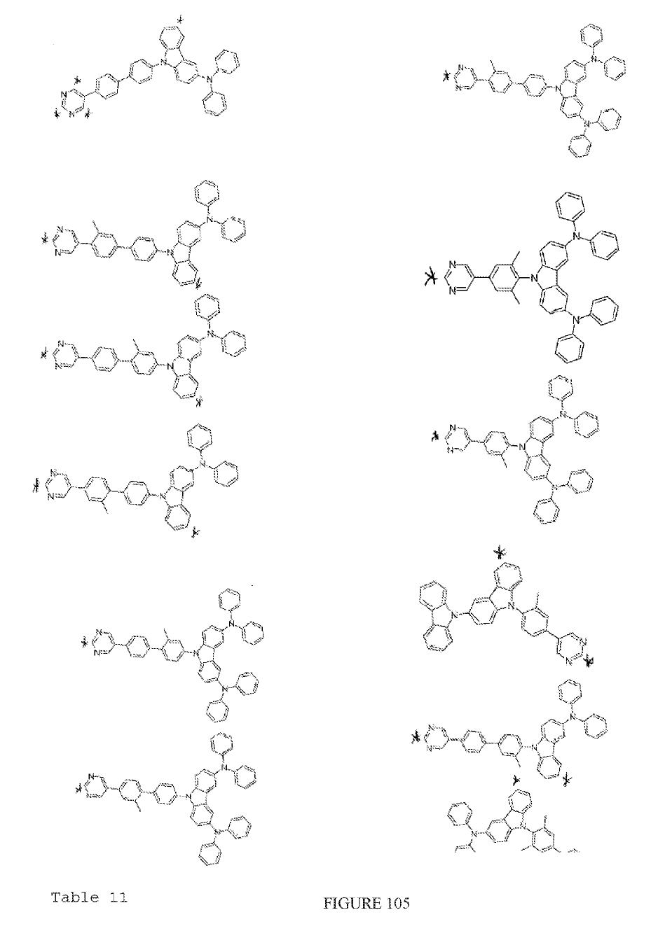

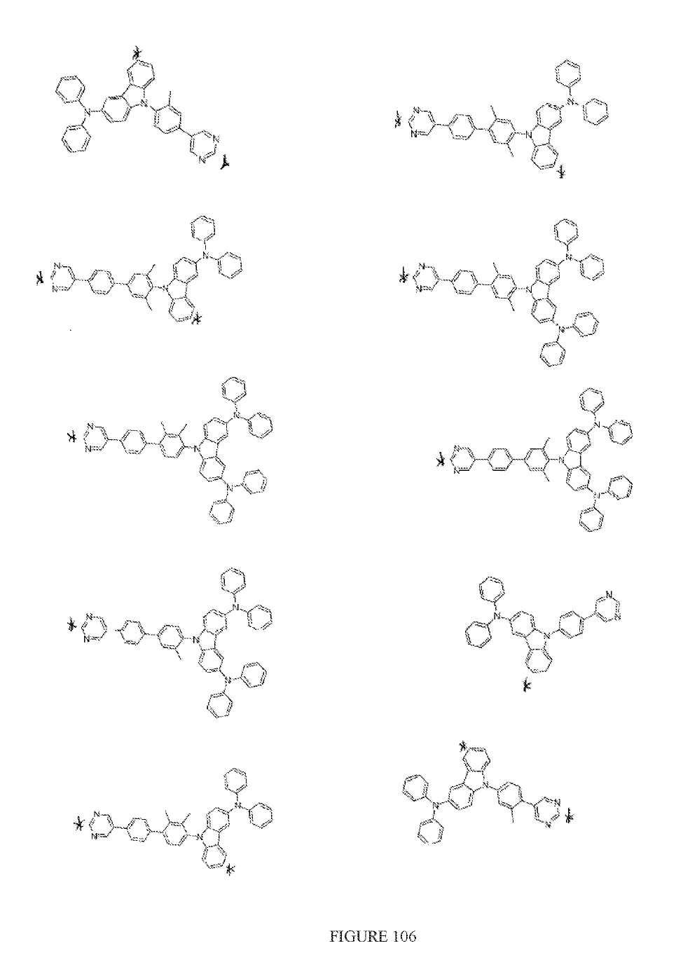

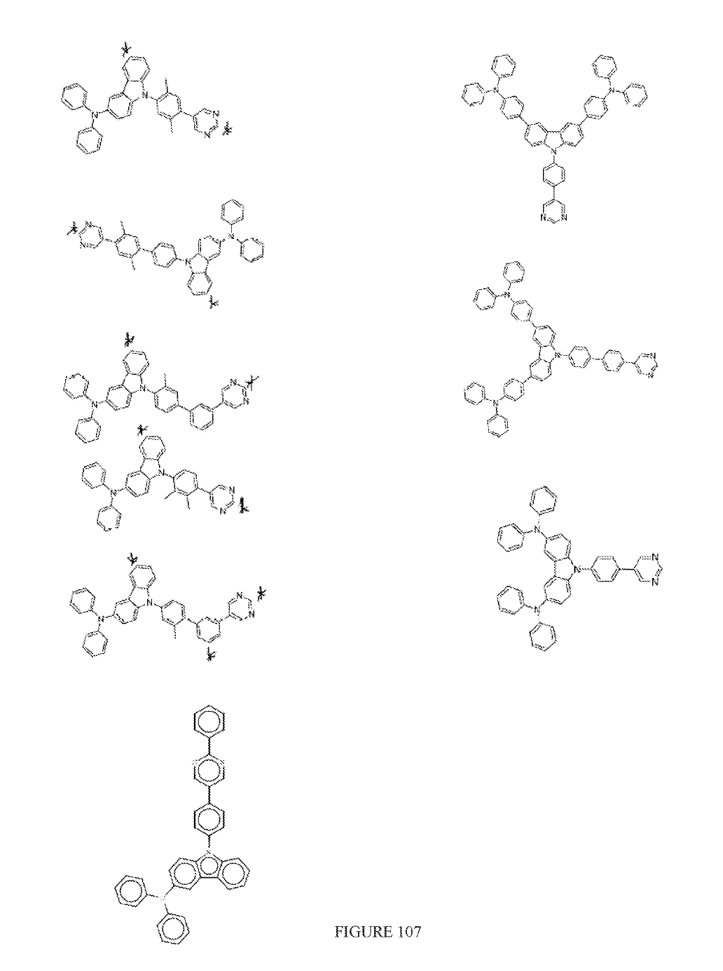

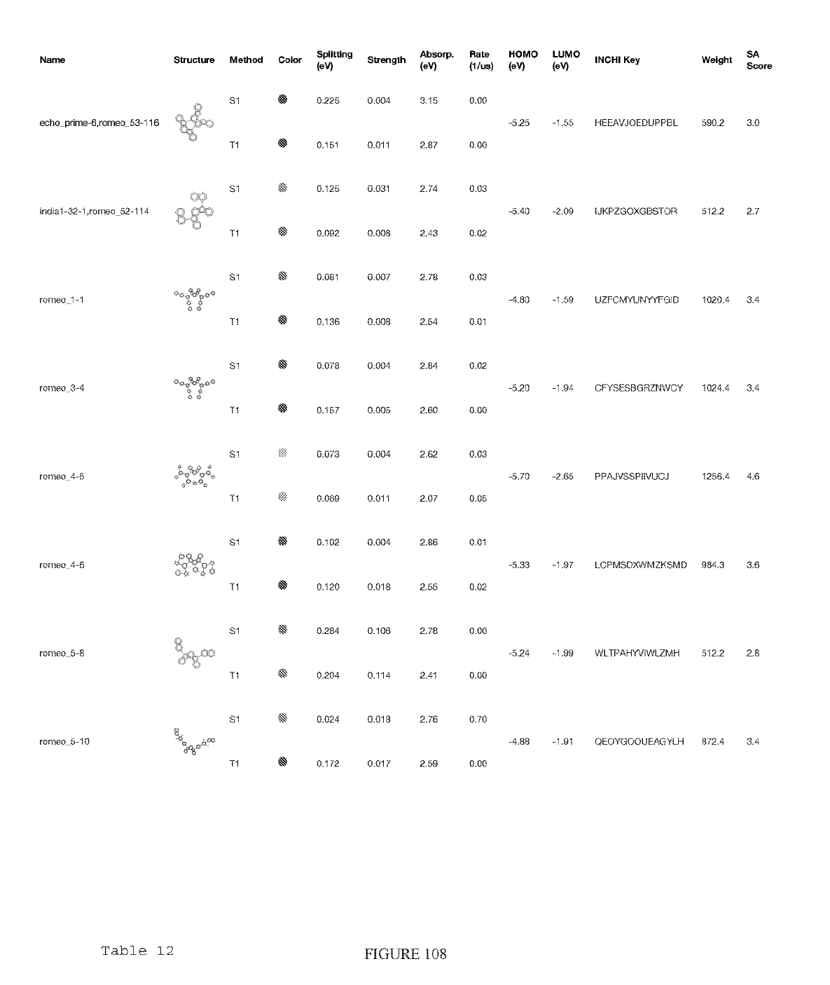

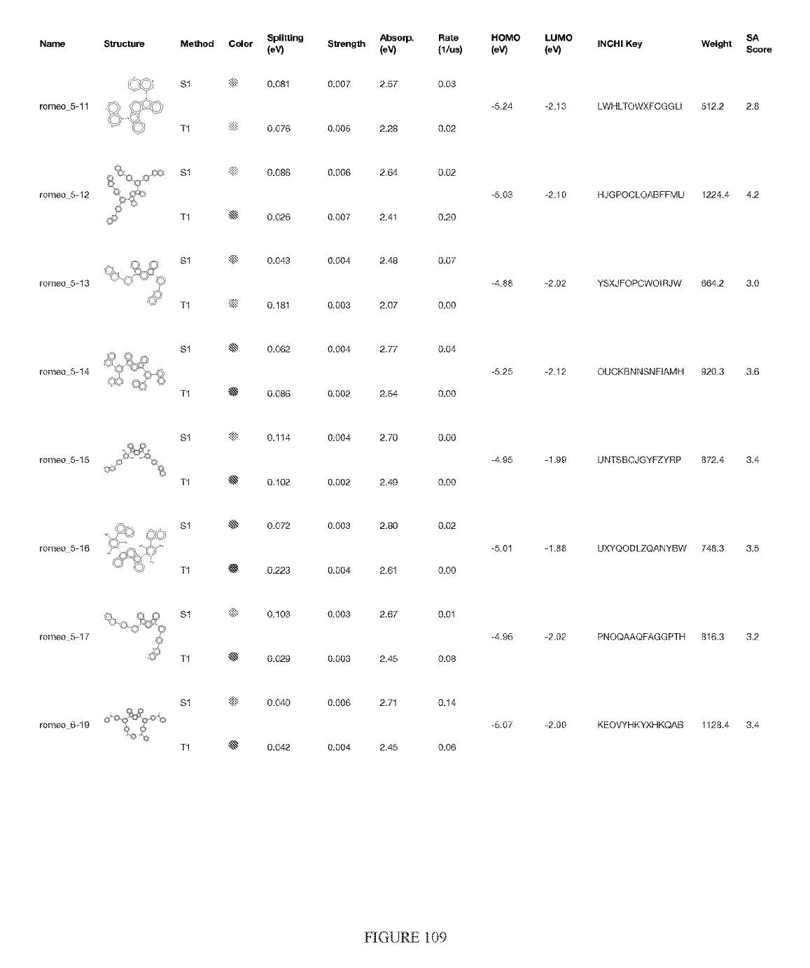

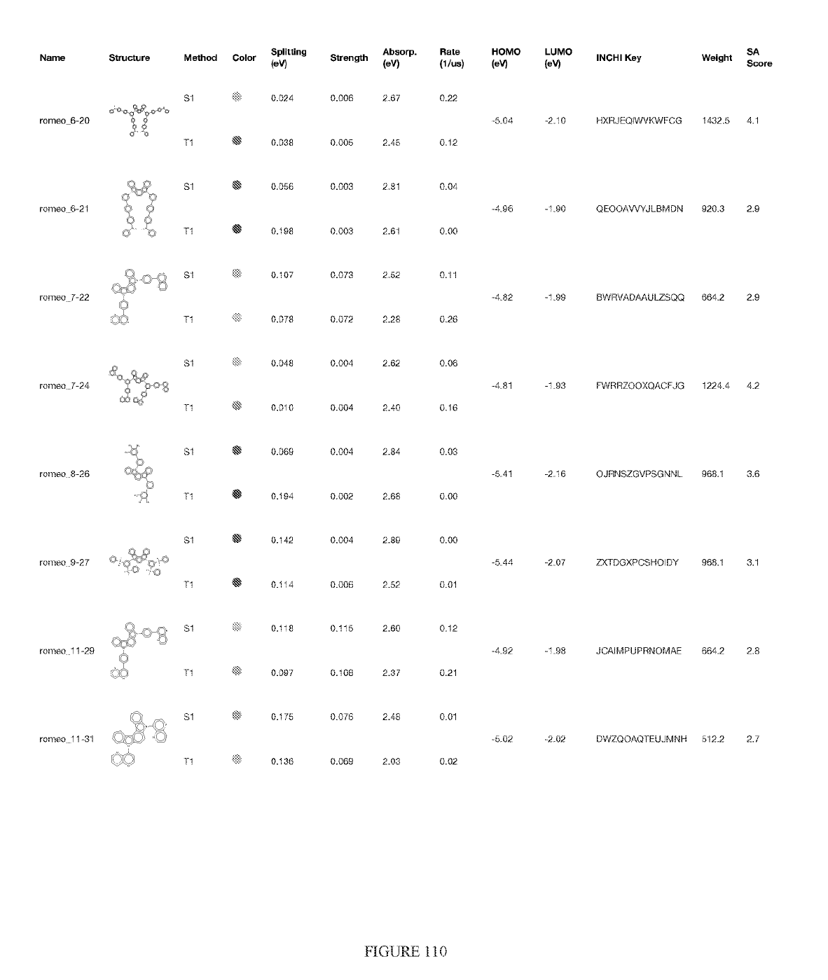

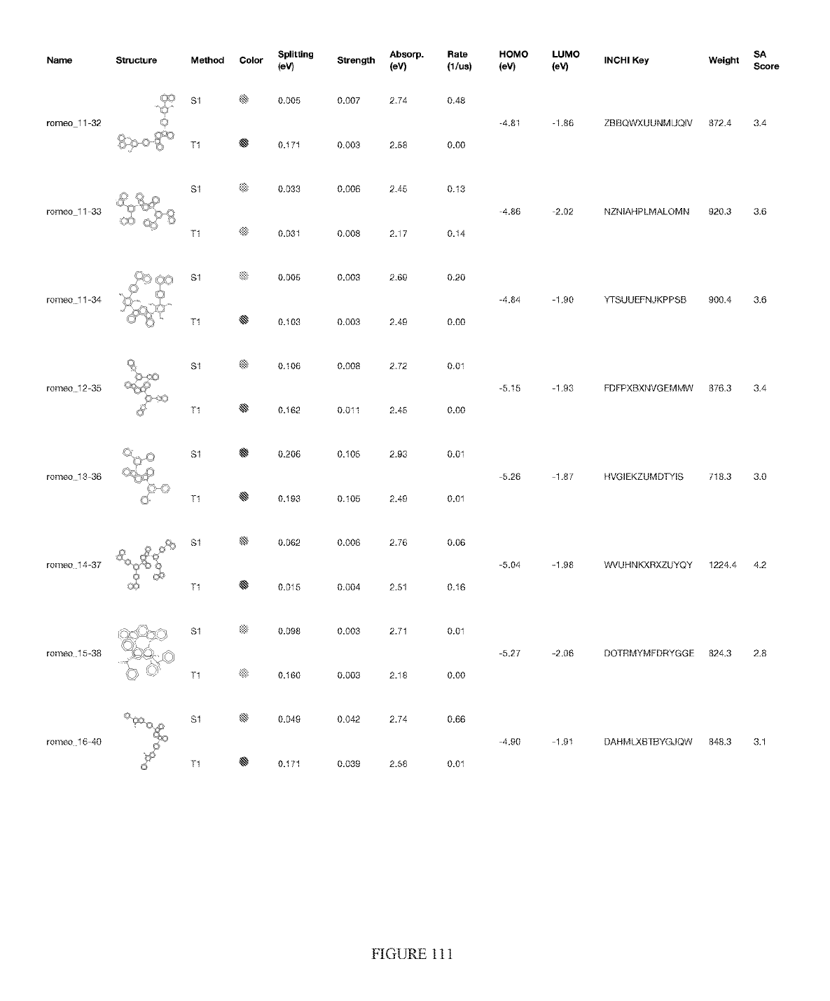

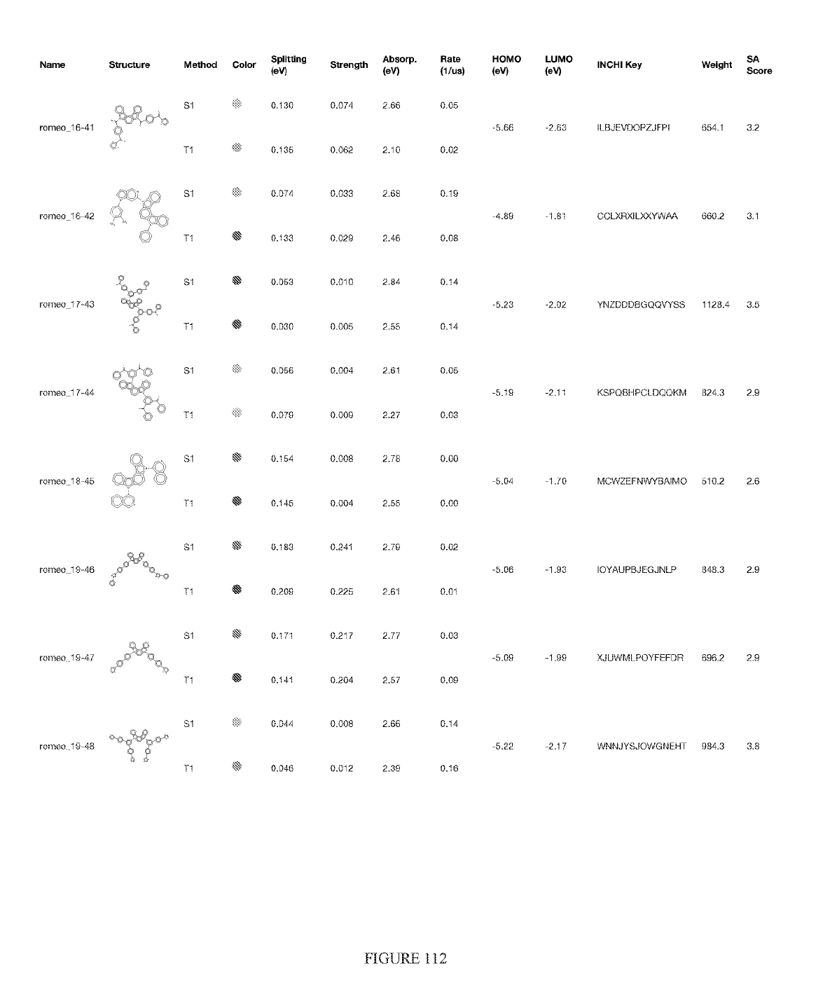

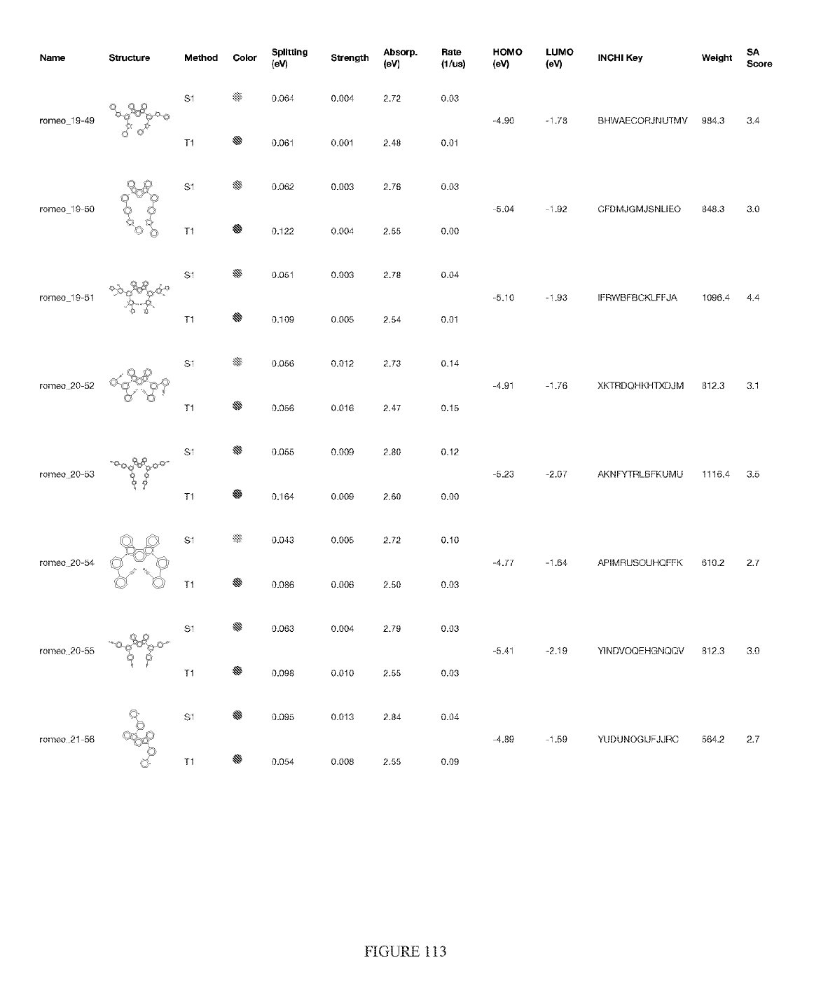

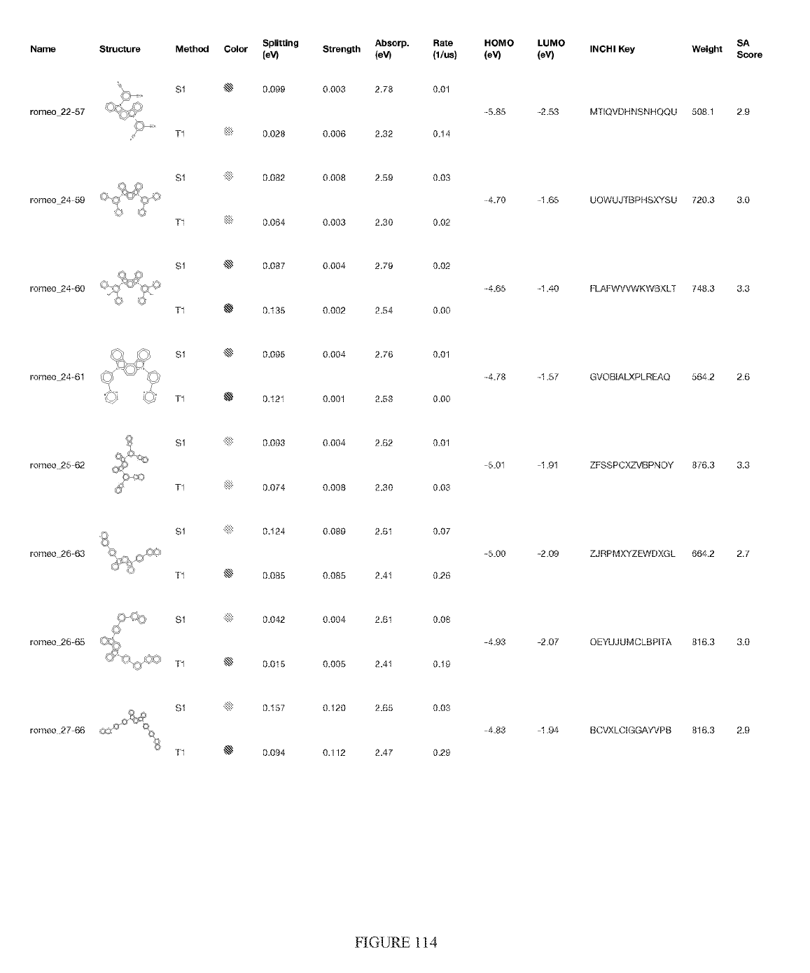

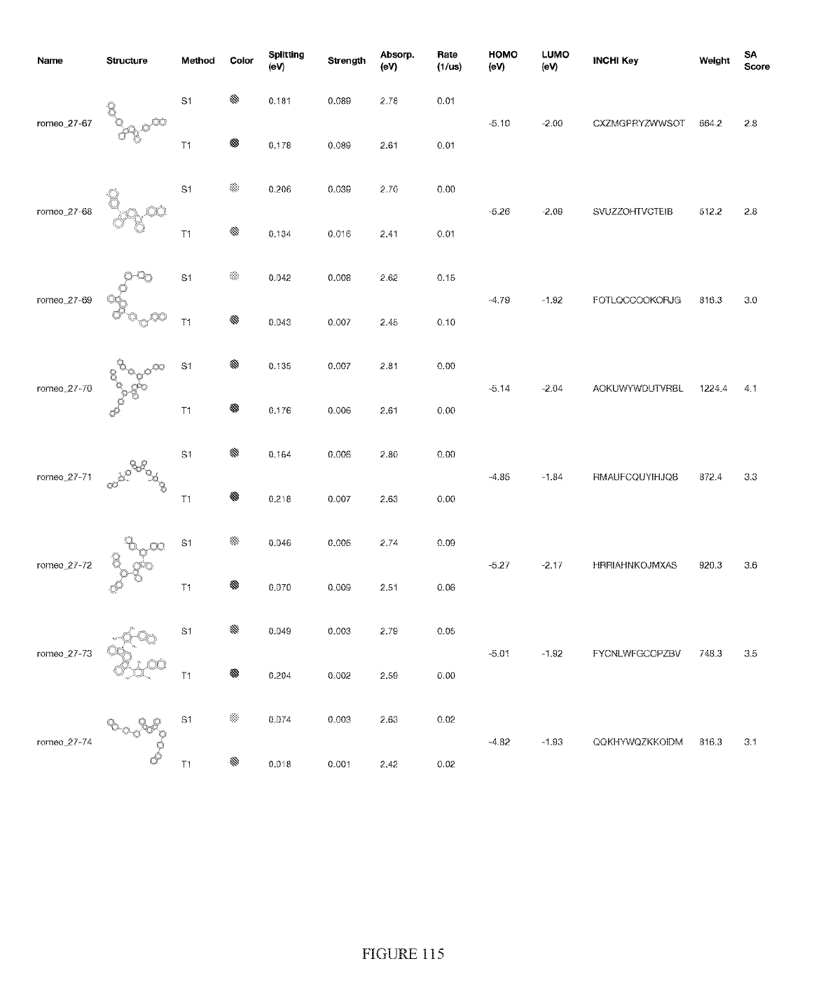

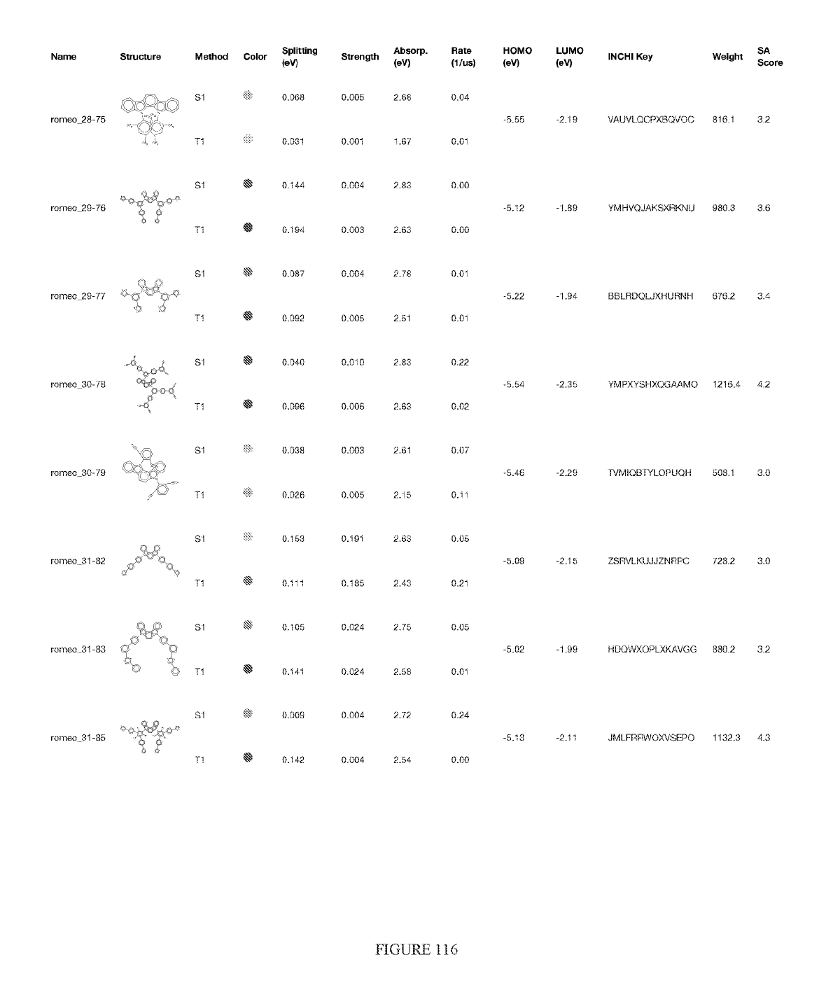

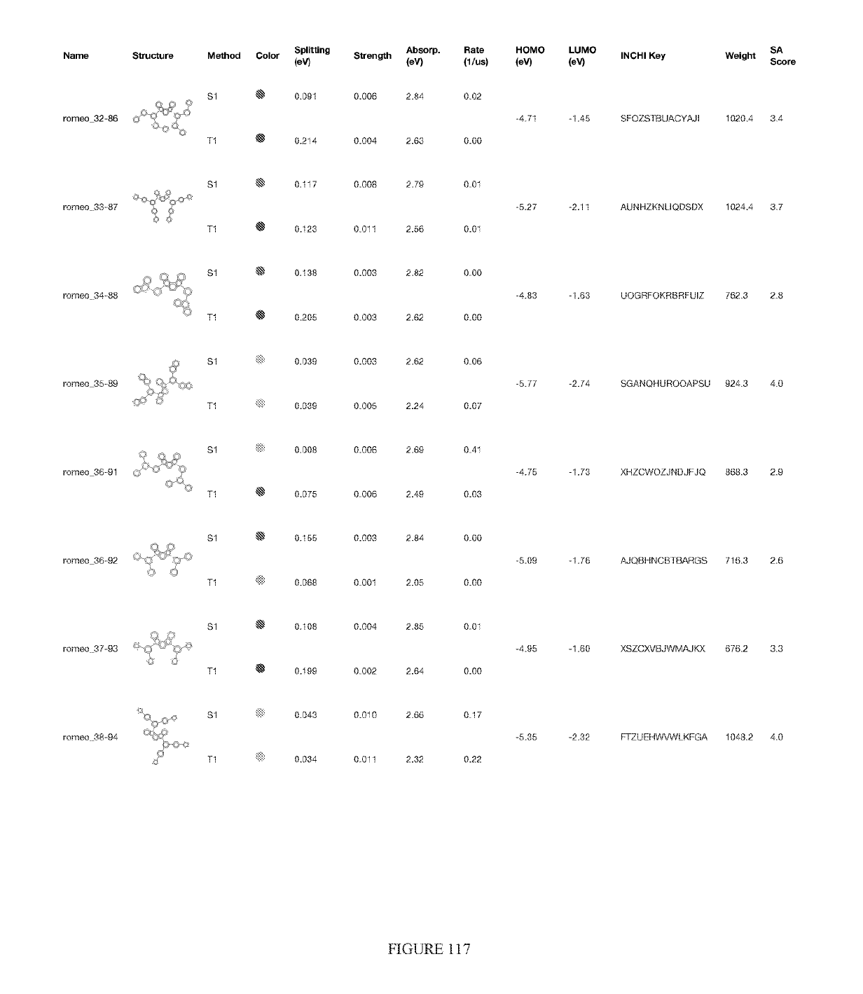

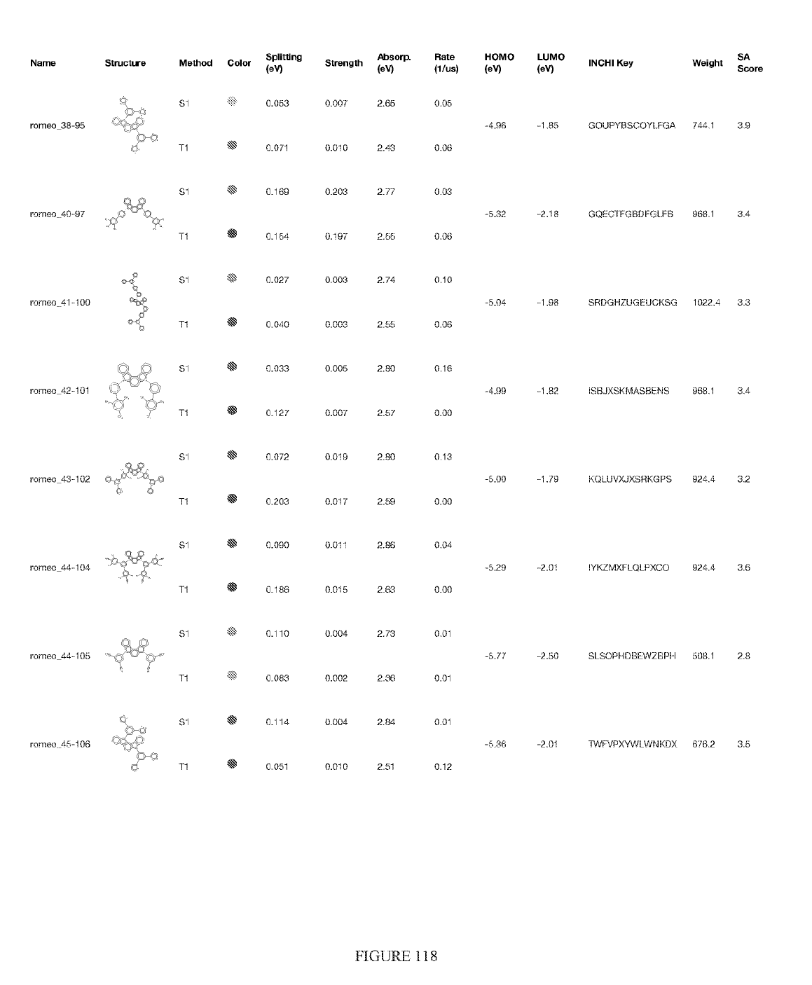

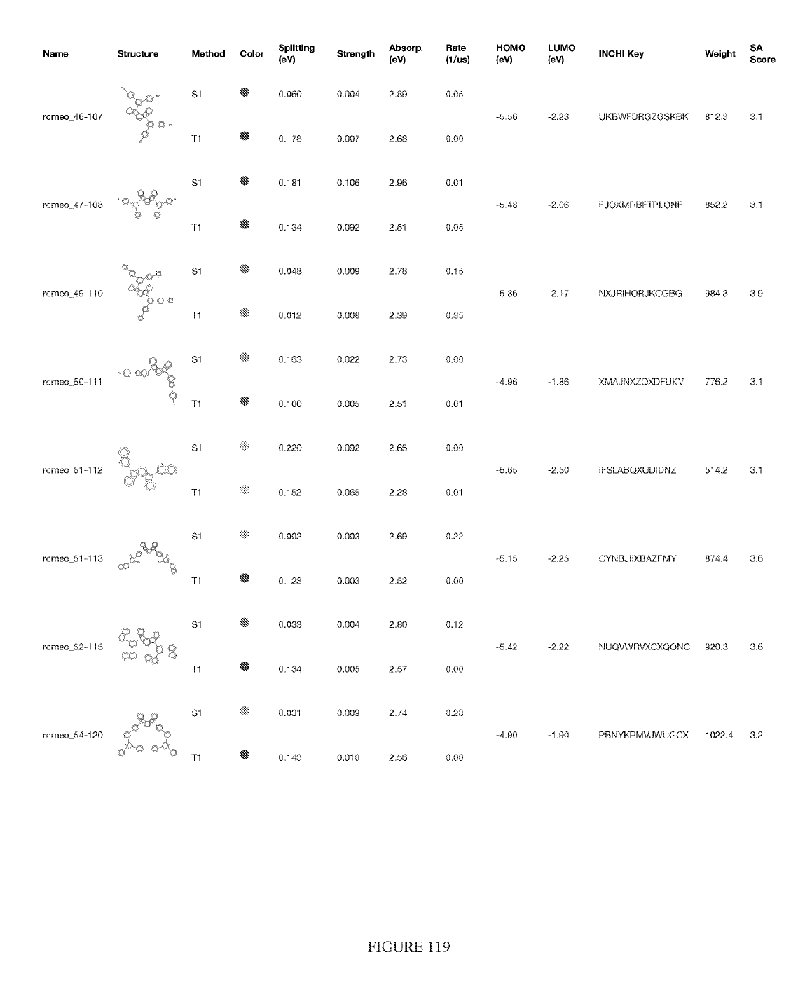

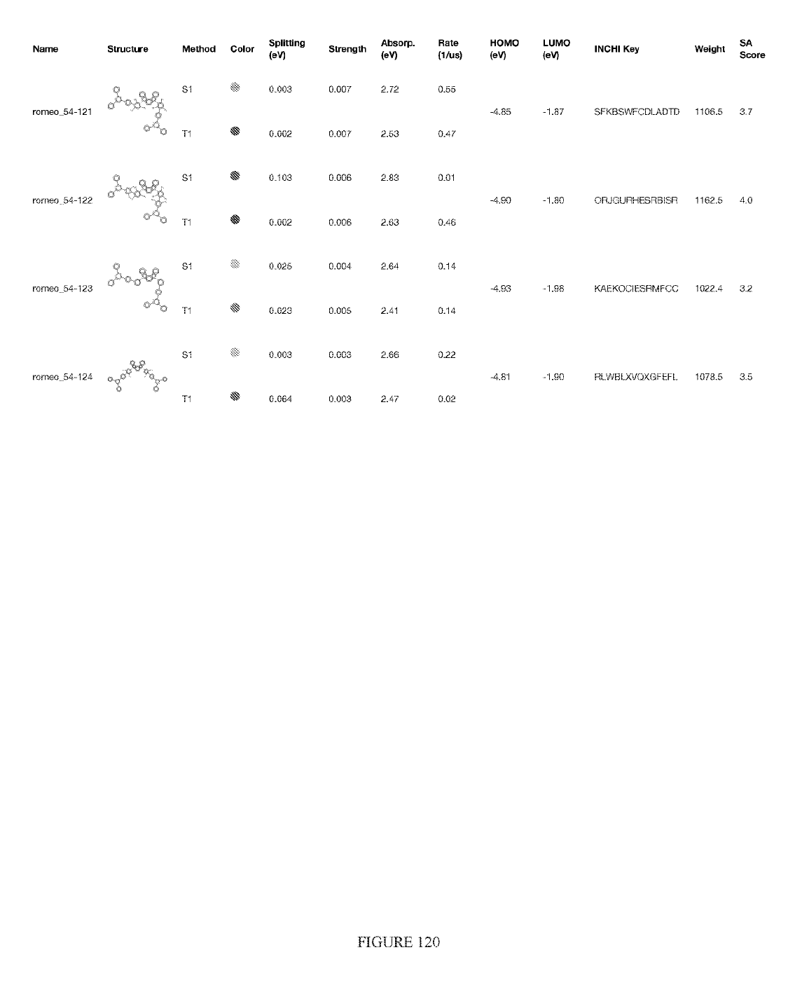

Further, recent research suggests that traditional Iridium based OLEDs may have reached a physical performance limit. As illustrated in FIG. 1, the brightness of an OLED will decrease as the time of decay increases. Since the highest energy triplet state is the origin of the luminescent transition in the Ir-based materials of FIG. 1, increasing the zero-field splitting through additional spin-orbit coupling will eventually lengthen the effective lifetime of the other two triplets. It is believed that this effect is responsible for the asymptote empirically observed at about 1 μs.

The recently developed thermally activated delayed fluorescence (TADF) seeks to minimize energetic splitting between singlet and triplet states (Δ). The reduction in exchange splitting from typical values of 0.4-0.7 eV to a gap of the order of the thermal energy (proportional to k_{B}T, where k_{B} represents the Boltzmann constant, and T represents temperature) means that thermal agitation can transfer population between singlet levels and triplet sublevels in a relevant timescale even if the coupling between states is small.

Example TADF molecules consist of donor and acceptor moieties connected directly by a covalent bond or via a conjugated linker (or “bridge”). A “donor” moiety is likely to transfer electrons from its HOMO upon excitation to the “acceptor” moiety. An “acceptor” moiety is likely to accept the electrons from the “donor” moiety into its LUMO. The donor-acceptor nature of TADF molecules results in low-lying excited states with charge-transfer character that exhibit very low Δ. Since thermal molecular motions can randomly vary the optical properties of donor-acceptor systems, a rigid three-dimensional arrangement of donor and acceptor moieties can be used to limit the non-radiative decay of the charge-transfer state by internal conversion during the lifetime of the excitation.

It is beneficial, therefore, to decrease energetic splitting between singlet and triplet states (Δ), and to create a system with increased reversed intersystem crossing (RISC) capable of exploiting triplet excitons. Such a system, it is believed, will result in decreased emission lifetimes. Systems with these features will be capable of emitting blue light without being subject to the rapid degradation prevalent in blue OLEDs known today.

### Compounds of the Invention

The molecules of the present invention, when excited via thermal or electronic means, can produce light in the blue or green region of the visible spectrum. The molecules comprise molecular fragments including at least one donor moiety, at least one acceptor moiety, and optionally, a bridge moiety.

Electronic properties of the example molecules of the present invention can be computed using known ab initio quantum mechanical computations. By scanning a library of small chemical compounds for specific quantum properties, molecules can be constructed which exhibit the desired spin-orbit/thermally activated delayed fluorescence (SO/TADF) properties described above.

It could be beneficial, for example, to build molecules of the present invention using molecular fragments with a calculated triplet state above 2.75 eV. Therefore, using a time-dependent density functional theory using, as a basis set, the set of functions known as 6-31G* and a Becke, 3-parameter, Lee-Yang-Parr hybrid functional to solve Hartree-Fock equations (TD-DFT/B3LYP/6-31G*), molecular fragments (moieties) can be screened which have HOMOs above a specific threshold and LUMOs below a specific threshold, and wherein the calculated triplet state of the moieties is above 2.75 eV.

Therefore, for example, a donor moiety (“D”) can be selected because it has a HOMO energy (e.g., an ionization potential) of greater than or equal to −6.5 eV. An acceptor moiety (“A”) can be selected because it has, for example, a LUMO energy (e.g., an electron affinity) of less than or equal to −0.5 eV. The bridge moiety (“B”) can be a rigid conjugated system which can, for example, sterically restrict the acceptor and donor moieties into a specific configuration, thereby preventing the overlap between the conjugated π system of donor and acceptor moieties.

Accordingly, in a first aspect, the present invention is a molecule comprising at least one acceptor moiety A, at least one donor moiety D, and optionally, a bridge moiety B. The moiety D, for each occurrence independently, is a monocyclic or fused polycyclic aryl or heteroaryl having between 5 and 20 atoms, optionally substituted with one or more substituents. The moiety A, for each occurrence independently, is —CF₃, —CN, or a monocyclic or fused polycyclic aryl or heteroaryl having between 5 and 20 atoms, optionally substituted with one or more substituents. The moiety B, for each occurrence independently, is phenyl optionally substituted with one to four substituents. Each moiety A is covalently attached to either the moiety B or the moiety D, each moiety D is covalently attached to either the moiety B or the moiety A, and each moiety B is covalently attached to at least one moiety A and at least one moiety D. At least one moiety A is selected from list AN1 or at least one moiety D is selected from list DN1.

### List AN1

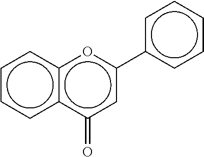

### List DN1

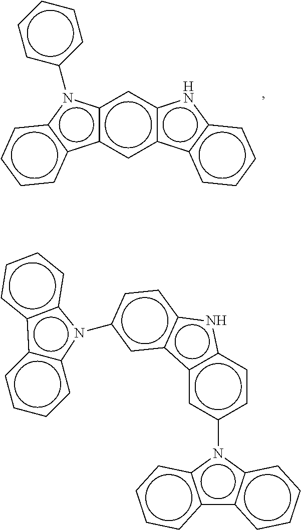

In an example embodiment of the first aspect, each moiety A is bonded either to moiety B or moiety D, each moiety B is bonded either to moiety A, moiety D, or a second moiety B, and each moiety D is bonded either to moiety A or moiety B. In another example embodiment of the first aspect, the moieties A are different than the moieties D.

The foregoing rules of connection mean that the moiety A cannot be connected to another moiety A, the moiety D cannot be connected to another moiety D, and that each moiety B is multivalent, and therefore must be connected to at least two other moieties, either a moiety A, a moiety D, or a second moiety B. It is understood that within a molecule no molecular fragment represented by A is the same as any molecular fragment represented by D.

In a second aspect, the present invention is a molecule comprising at least one acceptor moiety A, at least one donor moiety D, and optionally, one or more bridge moieties B; wherein A, D, and B are defined above with respect to the first aspect of the present invention, and wherein at least one moiety A is selected from list AN1 or at least one moiety D is selected from list DN1. In addition to the moieties recited above in the first aspect, the moiety D can be —N(C₆-C₁₈aryl)₂. In addition to the moieties recited above with respect to the first aspect, the moiety A, can be —S(O)₂—. In addition to the moieties recited above with respect to the first aspect, the moiety B can be C₂-C₆ alkenyl, C₂-C₆ alkynyl, or C₅-C₁₂ cycloalkyl optionally substituted with one to four substituents.

In a third aspect, the present invention is a molecule defined by the structural formula (G-I)

(A)ₘ-(B)₁-(D)ₚ (G-I)

wherein A, B, and D are defined above with respect to the first and second aspects, at least one moiety A is selected from list AN1, and at least one moiety D is selected from list DN1, and

the moiety D, for each occurrence independently, is optionally substituted with one or more substituents each independently selected from C₁-C₆ alkyl, C₂-C₆ alkenyl, C₂-C₆ alkynyl, C₆-C₁₈ aryl, (5-20 atom) heteroaryl, C₁-C₆ alkoxy, amino, C₁-C₃ alkylamino, C₁-C₃ dialkylamino, or oxo;

the moiety A, for each occurrence independently, is optionally substituted with one or more substituents independently selected from C₁-C₆ alkyl, C₂-C₆ alkenyl, C₂-C₆ alkynyl, C₆-C₁₈ aryl, (5-20 atom) heteroaryl, C₁-C₆ alkoxy, —C(O)C₁-C₃ haloalkyl, —S(O₂)H, —NO₂, —CN, oxo, halogen, or C₆-C₁₈ haloaryl;

the moiety B, for each occurrence independently, is optionally substituted with one to four substituents, each independently selected from C₁-C₆ alkyl, C₂-C₆ alkenyl, C₂-C₆ alkynyl, C₆-C₁₈ aryl, or (5-20 atom) heteroaryl;

m is an integer greater than 1;

p is an integer greater than 1; and

l is either 0 or an integer greater than one. In an example embodiment, l is greater than 1. In another example embodiment, 1 is 0, 1, or 2.

In a fourth aspect, the present invention is a molecule defined by the structural formula (G-I)

(A)ₘ-(B)ₗ-(D)ₚ (G-I)

wherein A, B, and D are defined above with respect to the first or second aspects of the present invention, at least one moiety A is selected from list AN1, and at least one moiety D is selected from list DN1, and

the moiety D, for each occurrence independently, is optionally substituted, in addition to the substituents described above with respect to the third aspect of the present invention, with —N(C₆-C₁₈ aryl)₂;

the moiety A, for each occurrence independently, is optionally substituted as described above with respect to the third aspect of the present invention;

the moiety B, for each occurrence independently, is optionally substituted as described above with respect to the third aspect of the present invention;

m is an integer greater than 1;

p is an integer greater than 1; and

l is either 0 or an integer greater than one. In an example embodiment, l is greater than 1. In another example embodiment, 1 is 0, 1, or 2.

In a fifth aspect, the present invention is molecule defined by the structural formula (G-I)

(A)ₘ-(B)ₗ-(D)ₚ (G-I)

wherein A, B, and D are defined above with respect to the first and second aspects of the present invention, at least one moiety A is selected from list AN1, and at least one moiety D is selected from list DN1, and

the moiety D, for each occurrence independently, is optionally substituted as described above with respect to the third and fourth aspects, and further wherein, each alkyl, alkenyl, alkynyl, aryl, and heteroaryl optionally further substituted with one or more substituents selected from C₁-C₆ alkyl, 5-20 atom heteroaryl, or —N(C₆-C₁₈aryl)₂;

the moiety A, for each occurrence independently, is optionally substituted as described above with respect to the third aspect of the present invention;

the moiety B, for each occurrence independently, is optionally substituted as described above with respect to the third aspect of the present invention;

m is an integer greater than 1;

p is an integer greater than 1; and

l is either 0 or an integer greater than one. In an example embodiment, l is greater than 1. In another example embodiment, 1 is 0, 1, or 2.

In a sixth aspect, the present invention is a molecule as defined above with respect to the first or second aspects of the present invention, and wherein the moiety D, for each occurrence independently, can be selected from List D1.

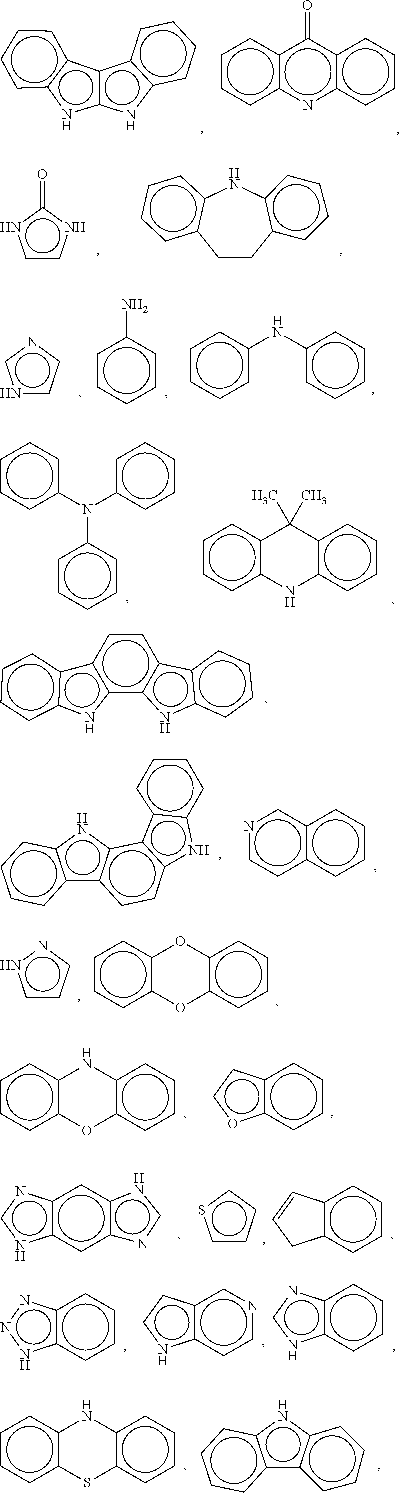

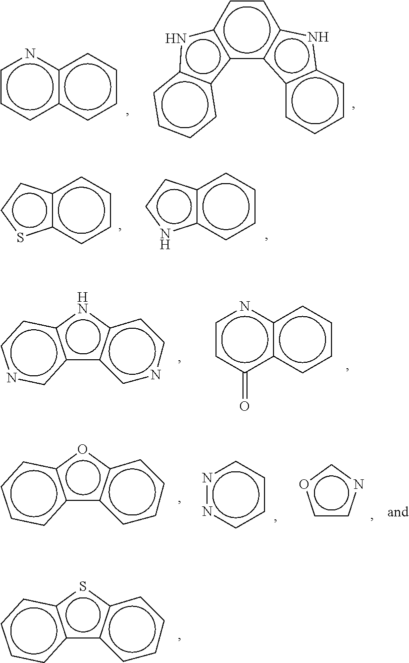

and wherein the moiety D can be optionally substituted as described above with respect to the third, fourth, and fifth aspects of the present invention.

In a seventh aspect, the present invention is a molecule as defined above with respect to the first or second aspects of the present invention, and wherein the moiety D, for each occurrence independently, can be selected from List D1, List D2, or both.

### List D2

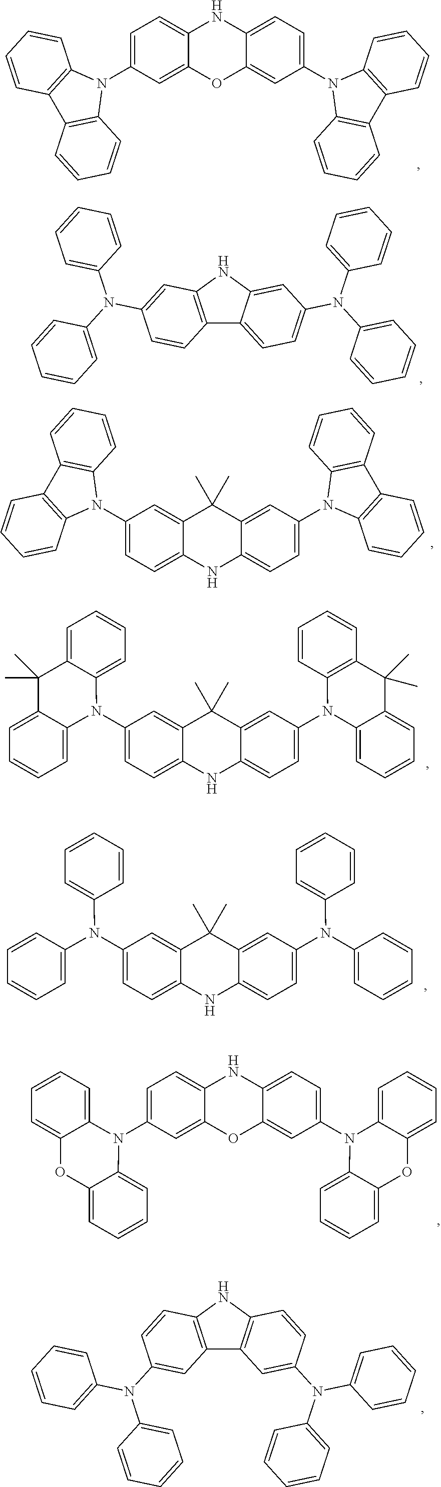

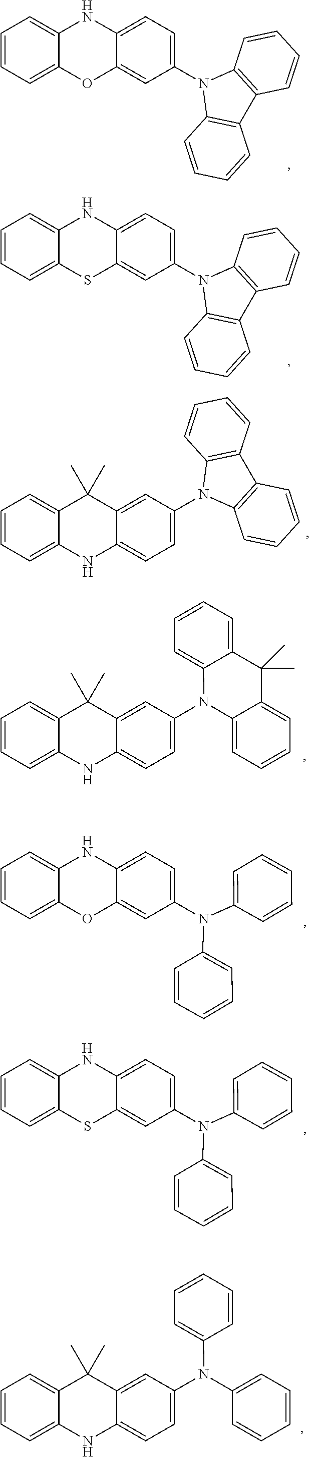

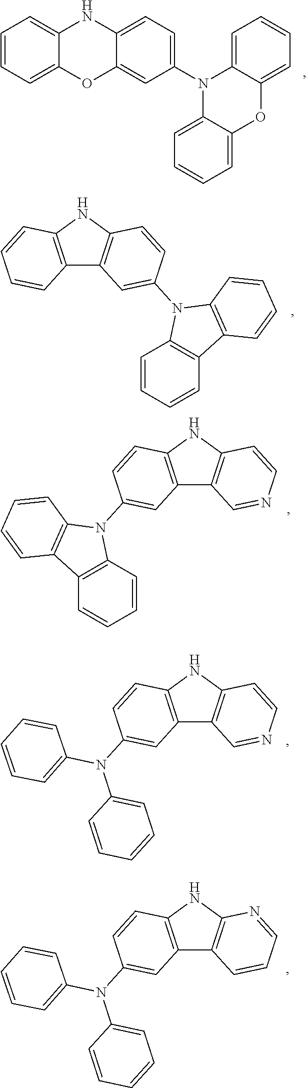

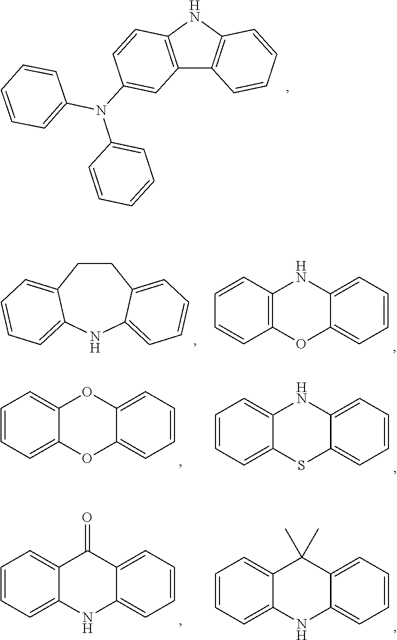

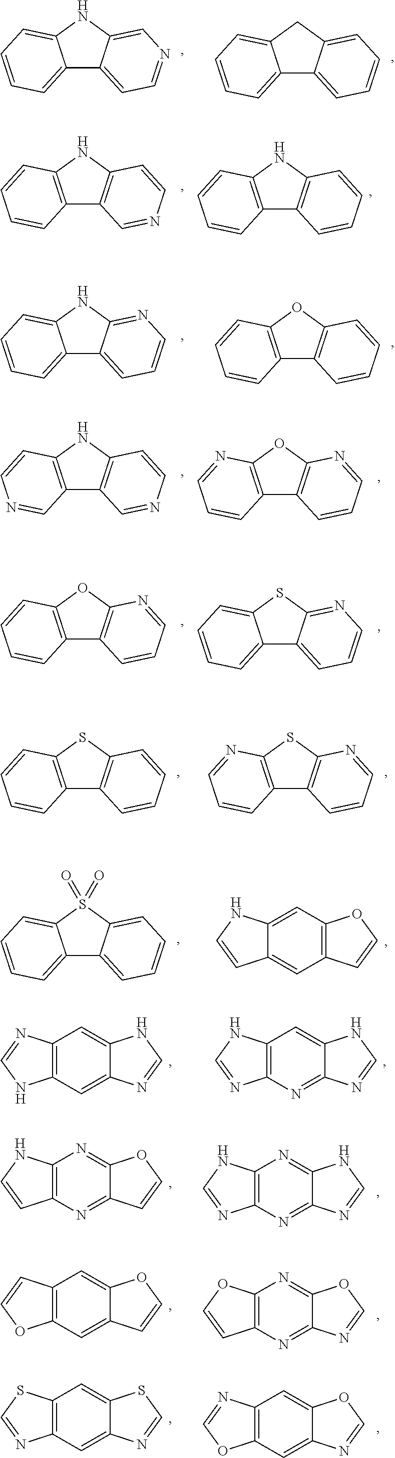

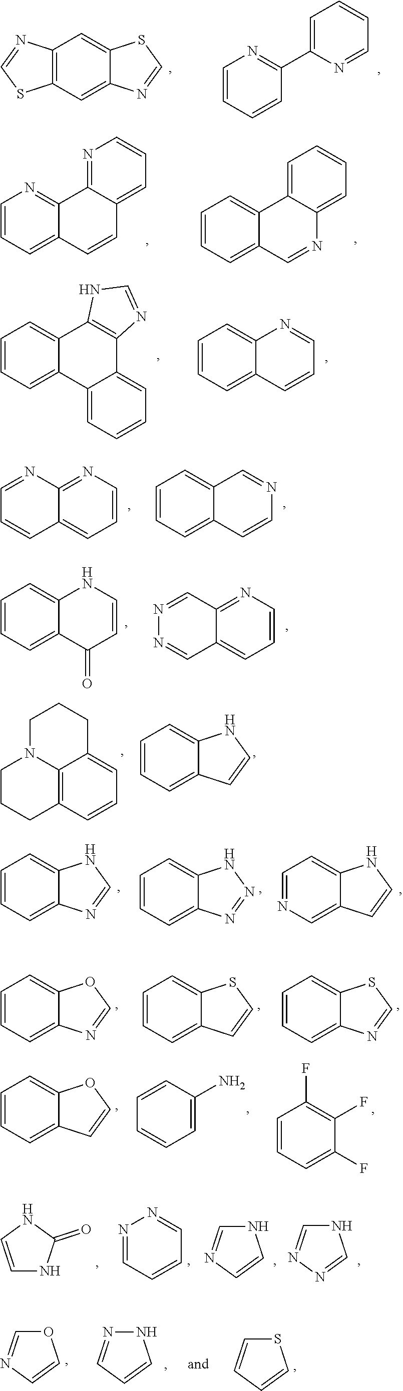

and wherein the moiety D can be optionally substituted as described above with respect to the third, fourth, and fifth aspects of the present invention.

In a eighth aspect, the present invention is a molecule as defined above with respect to the first or second aspects of the present invention, and wherein the moiety D, for each occurrence independently, can be selected from List D1, List D2, List D3, or any combination thereof.

### List D3

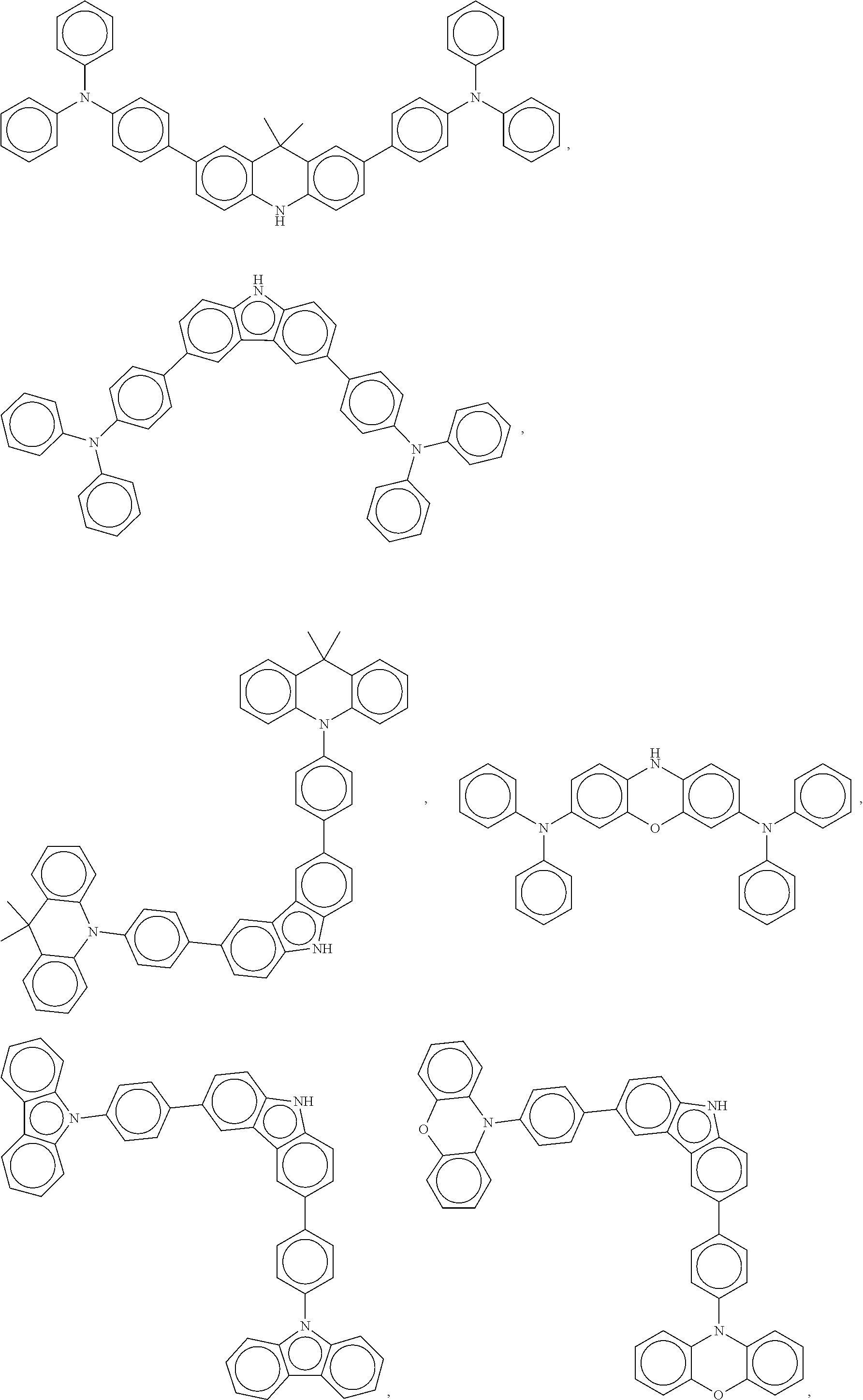

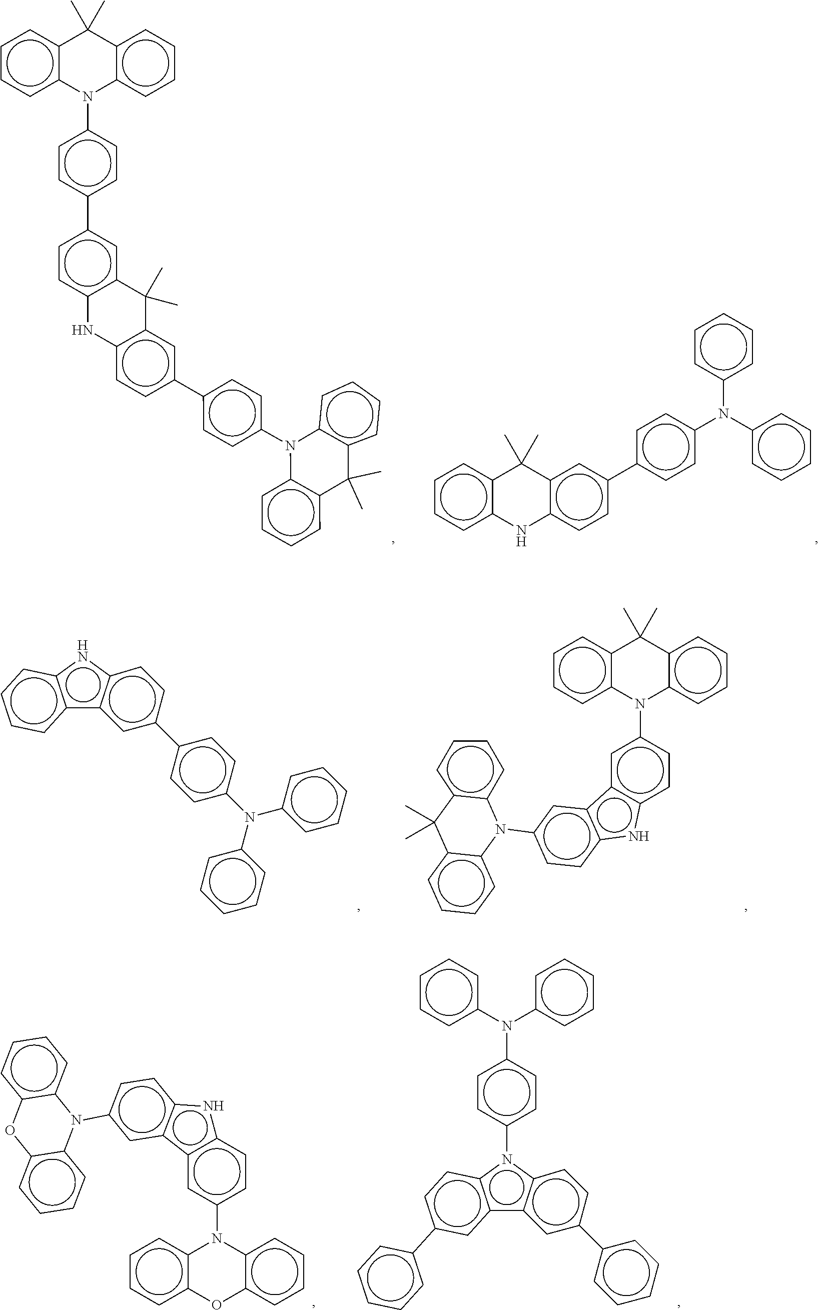

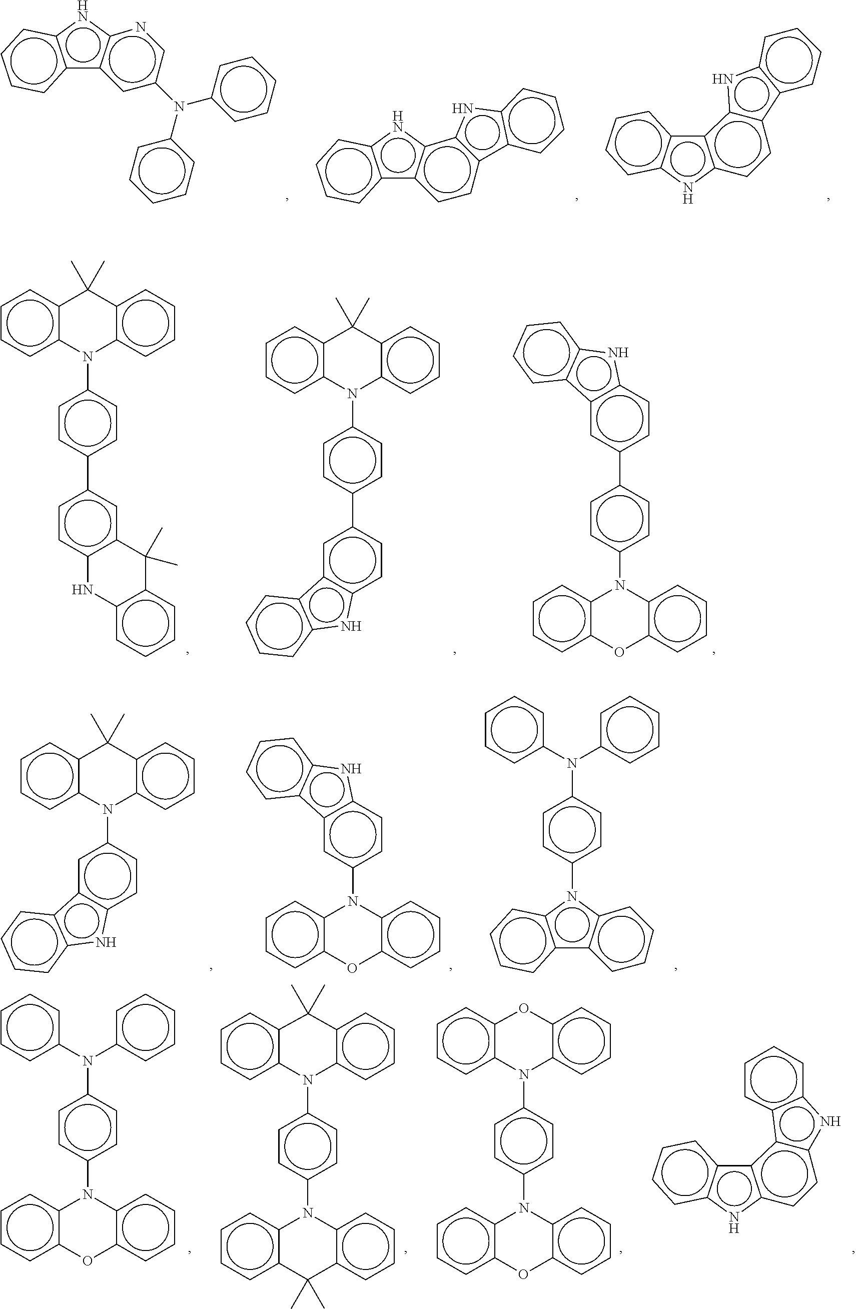

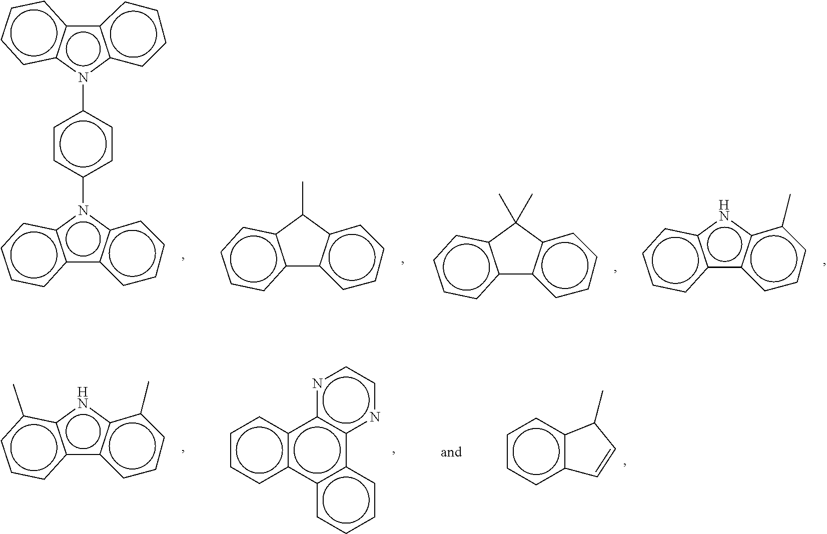

and wherein the moiety D can be optionally substituted as described above with respect to the third, fourth, and fifth aspects of the present invention.

In a ninth aspect, the present invention is a molecule as defined above with respect to the first or second aspects of the present invention, and wherein the moiety A, for each occurrence independently, can be selected from List A1.

### List A1

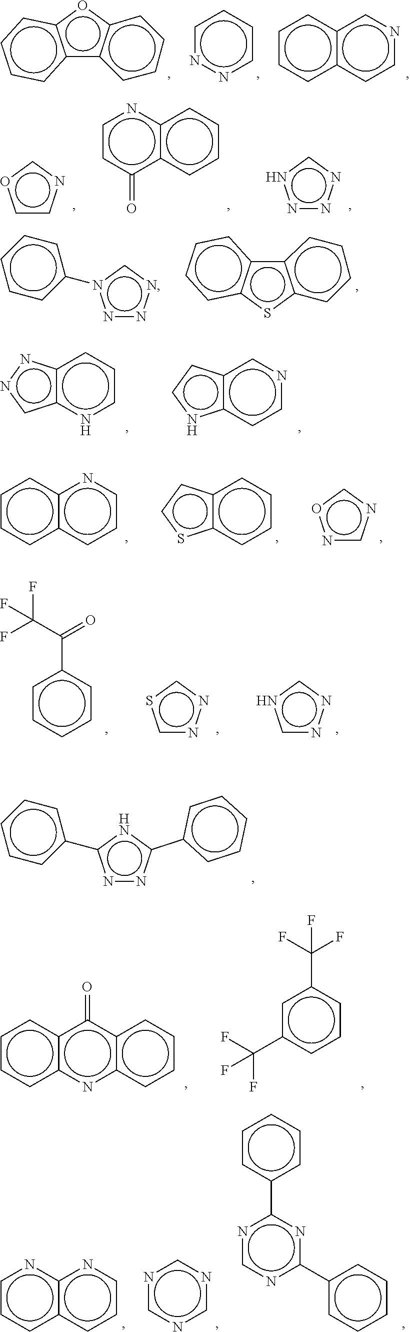

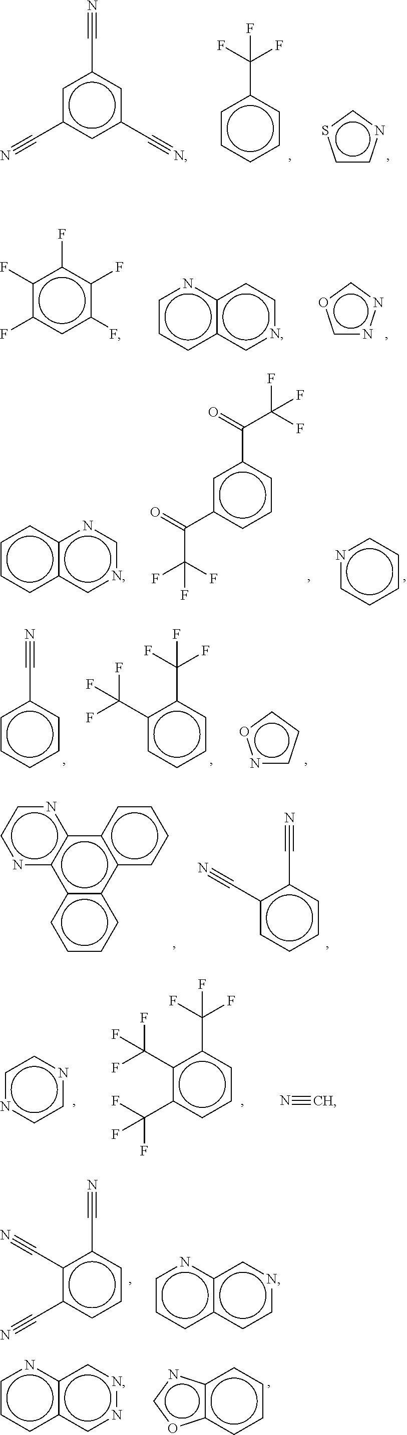

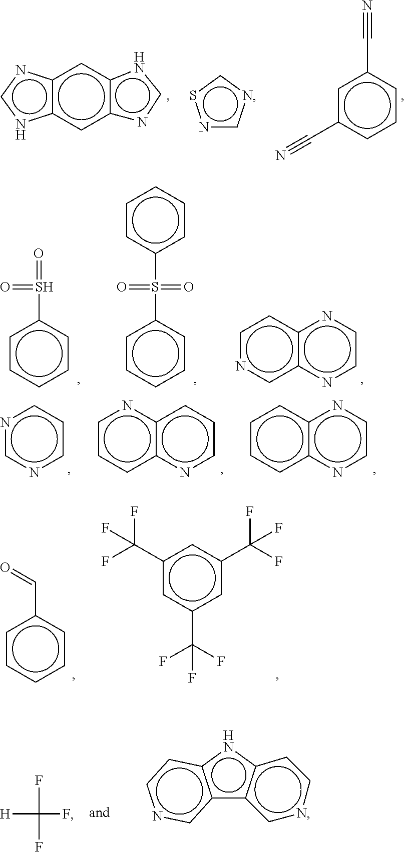

and wherein the moiety A can be optionally substituted as described above with respect to the third, fourth, and fifth aspects of the present invention.

In a tenth aspect, the present invention is a molecule as defined above with respect to the first, second, third, aspects of the present invention, and wherein the moiety A, for each occurrence independently, can be selected from List A1, List A2, or both.

### List A2

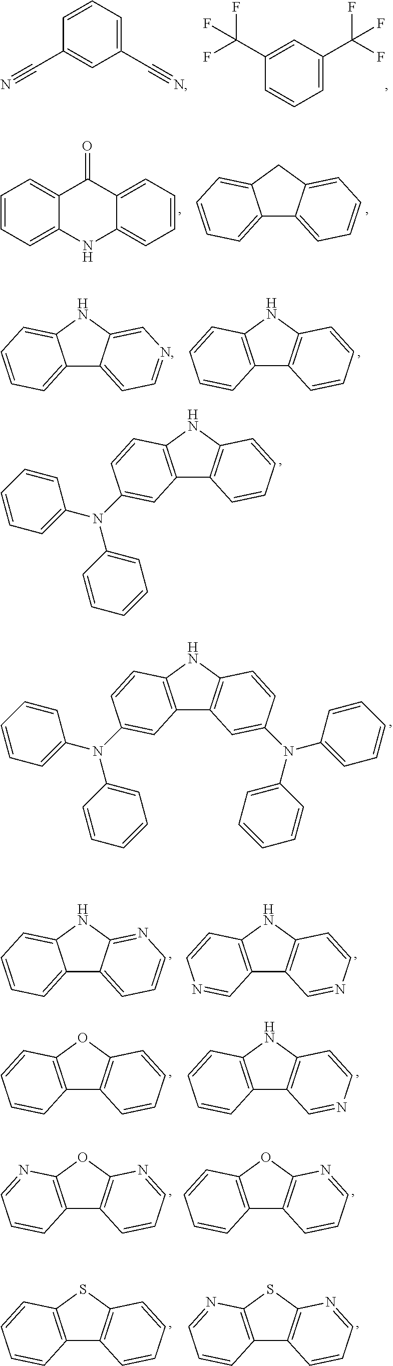

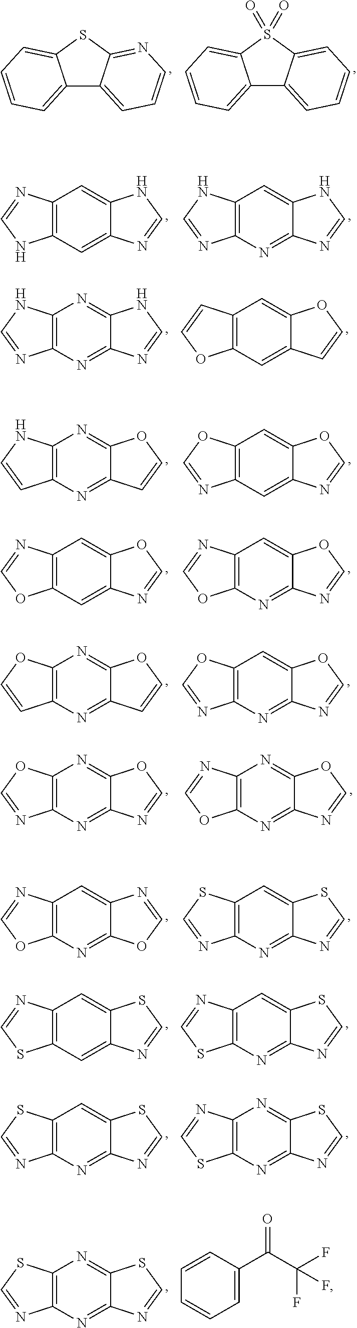

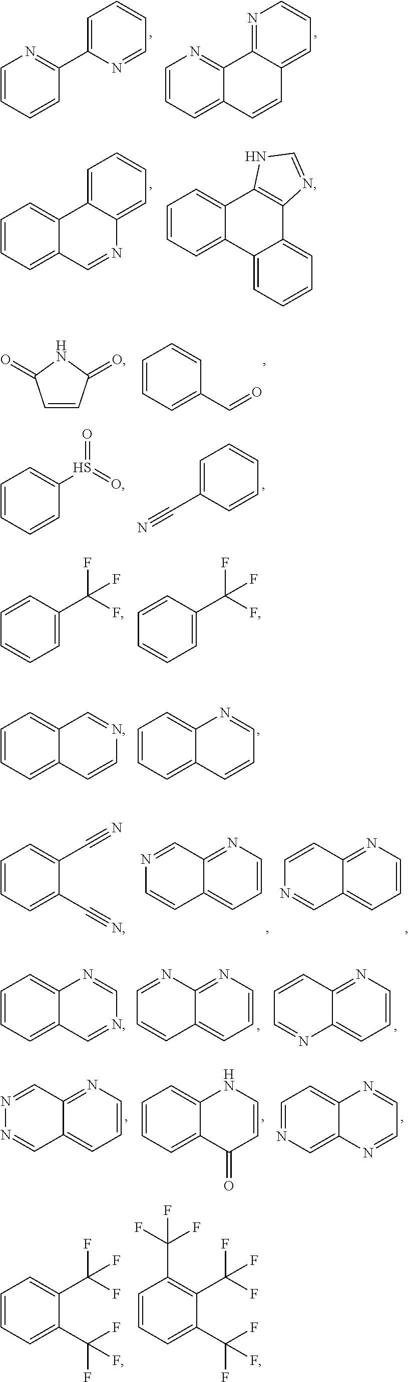

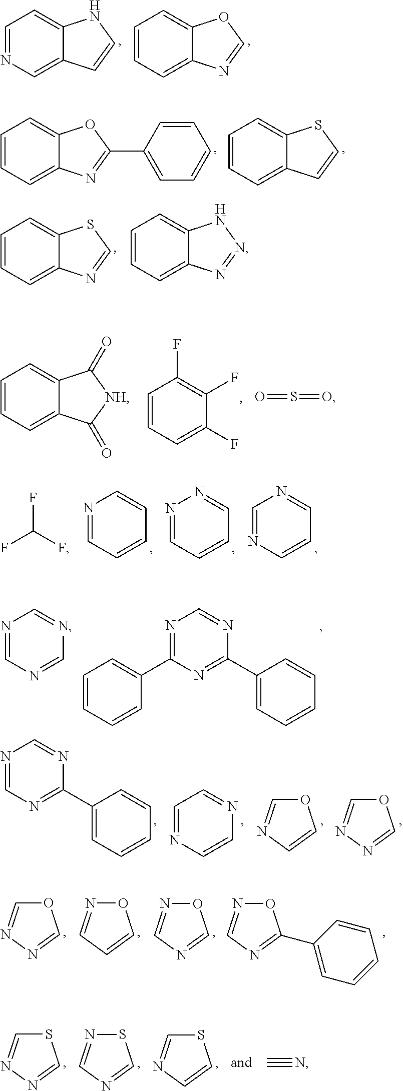

and wherein the moiety A can be optionally substituted as described above with respect to the third, fourth, and fifth aspects of the present invention.

In a eleventh aspect, the present invention is a molecule as defined above with respect to the first or second aspects of the present invention, and wherein the moiety A, for each occurrence independently, can be selected from List A1, List A2, List A3, or any combination thereof.

### List A3

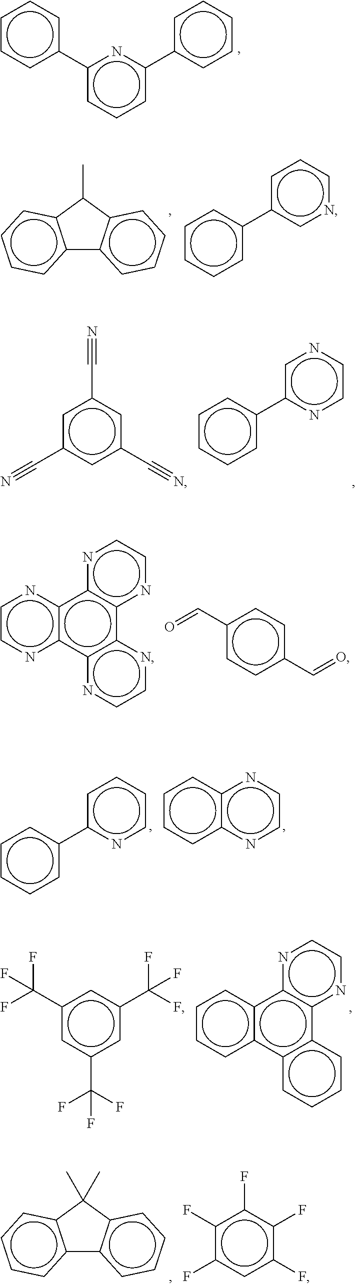

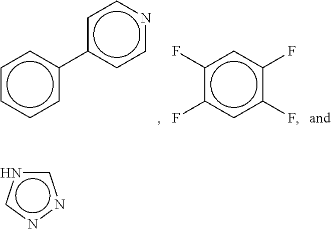

and wherein the moiety A can be optionally substituted as described above with respect to the third, fourth, and fifth aspects of the present invention.

In a twelfth aspect, the present invention is a molecule as defined above with respect to the first or second aspects of the present invention, and wherein the moiety B, for each occurrence independently, can be selected from List B1:

### List B 1

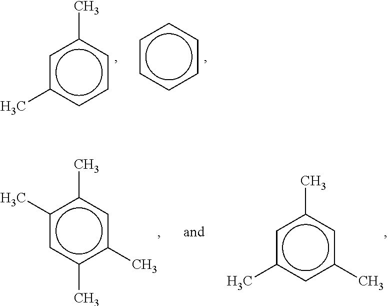

and wherein the moiety B can be optionally substituted as described above with respect to the third, fourth, and fifth aspects of the present invention.

In a thirteenth aspect, the present invention is a molecule as defined above with respect to the first or second aspects of the present invention, and wherein the moiety B, for each occurrence independently, can be selected from List B1, List B2, or both.

### List B2

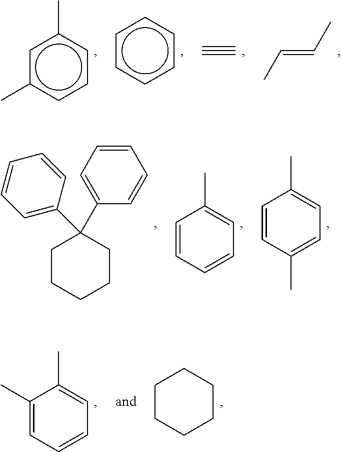

and wherein the moiety B can be optionally substituted as described above with respect to the third, fourth, and fifth aspects of the present invention.

In an example embodiment of the sixth aspect of the present invention, the moiety D, for each occurrence independently, is selected from List D4.

### List D4

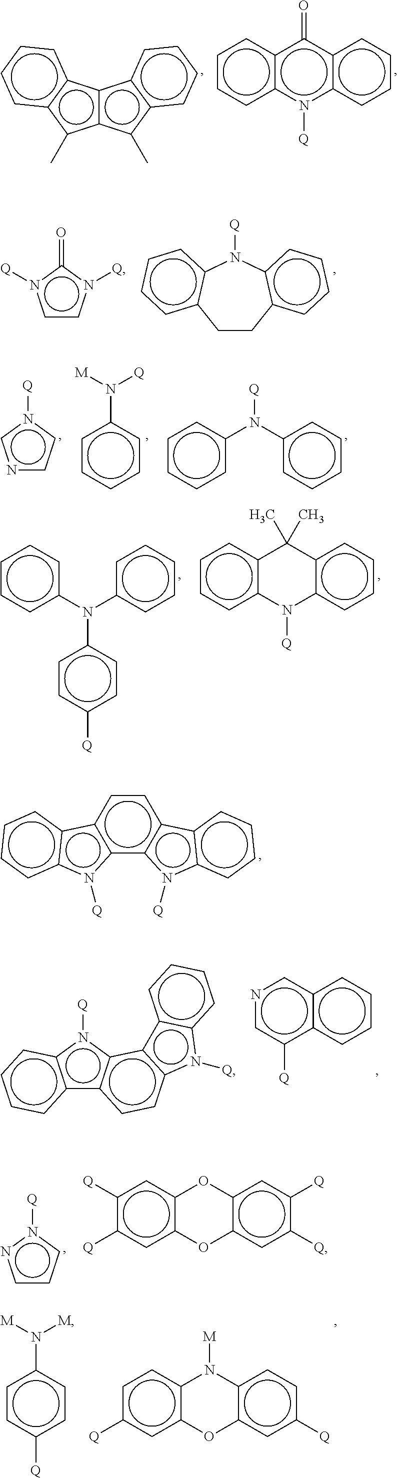

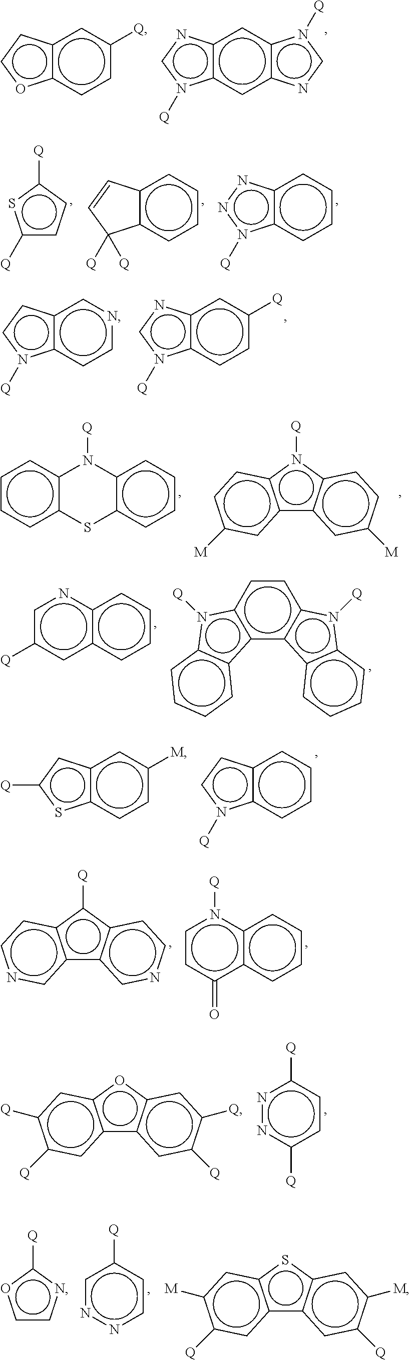

wherein, within each molecule:

Q is the moiety A or a moiety B₀₋₂-A and each M is the moiety A or the moiety B₀₋₂-A,

all groups Q are the same and all groups M are the same, and each group Q is the same or different from any group M, and the moieties A and B are defined above with respect to the first, second, and third aspects of the present invention.

In an example embodiment of the seventh aspect of the present invention, the moiety D, for each occurrence independently, is selected from List D4, List D5, or both.

### List D5

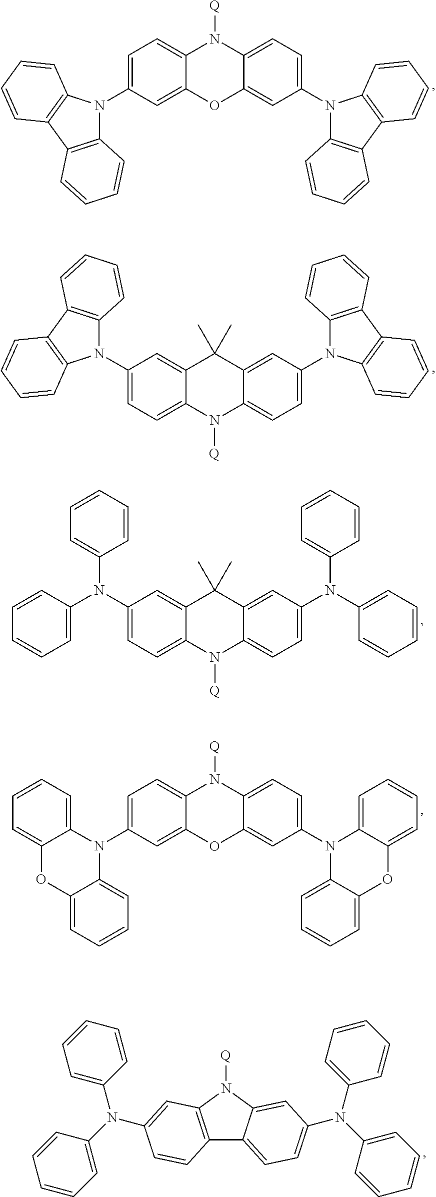

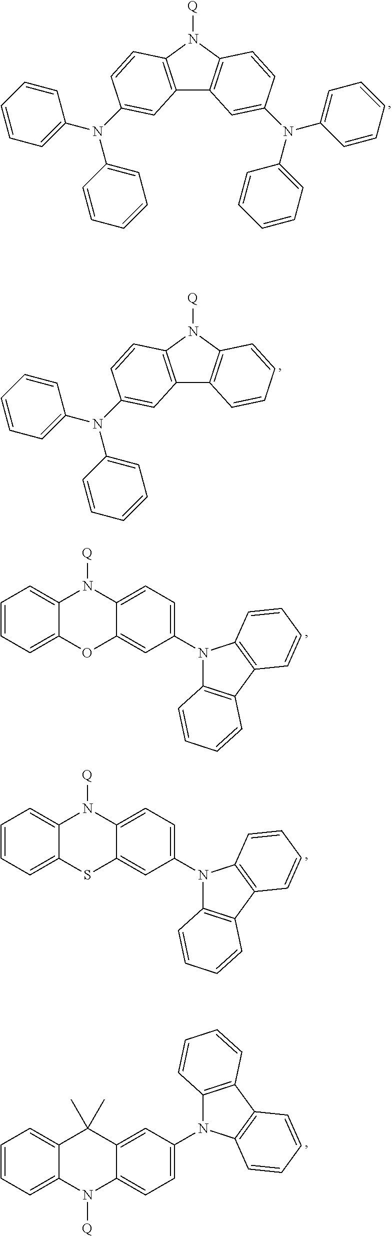

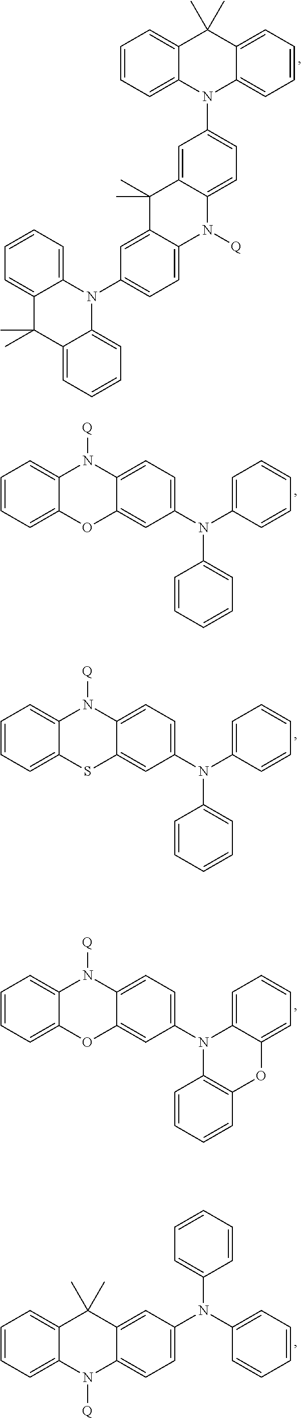

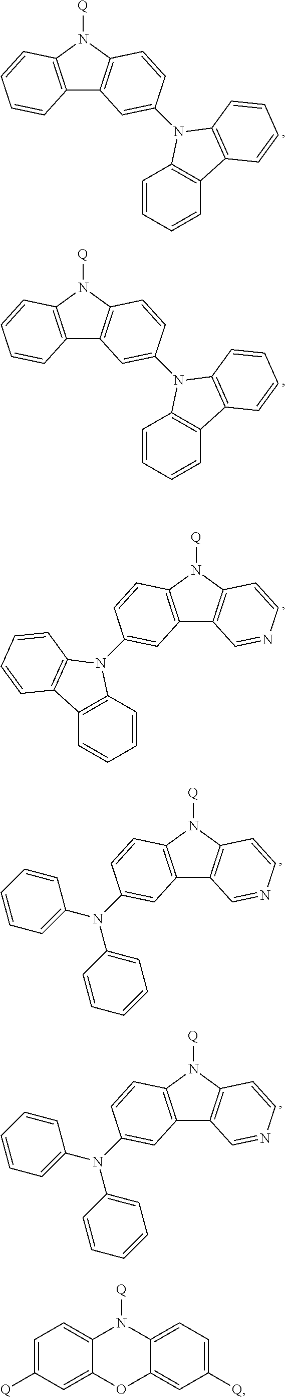

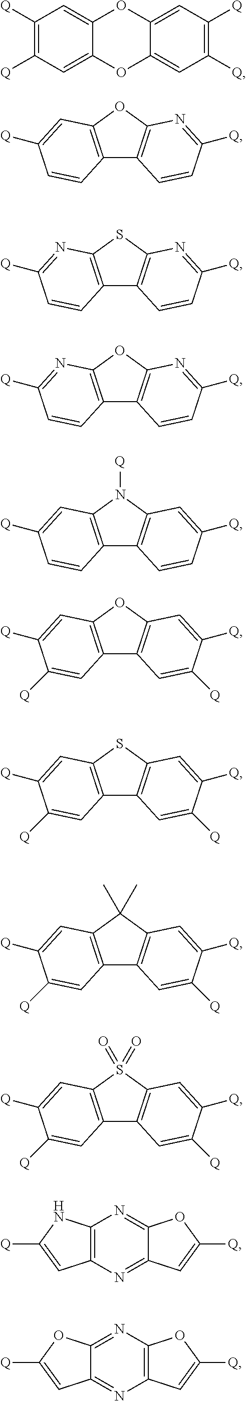

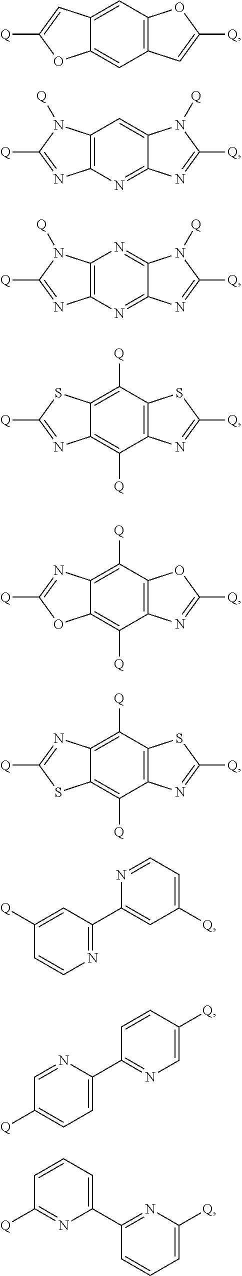

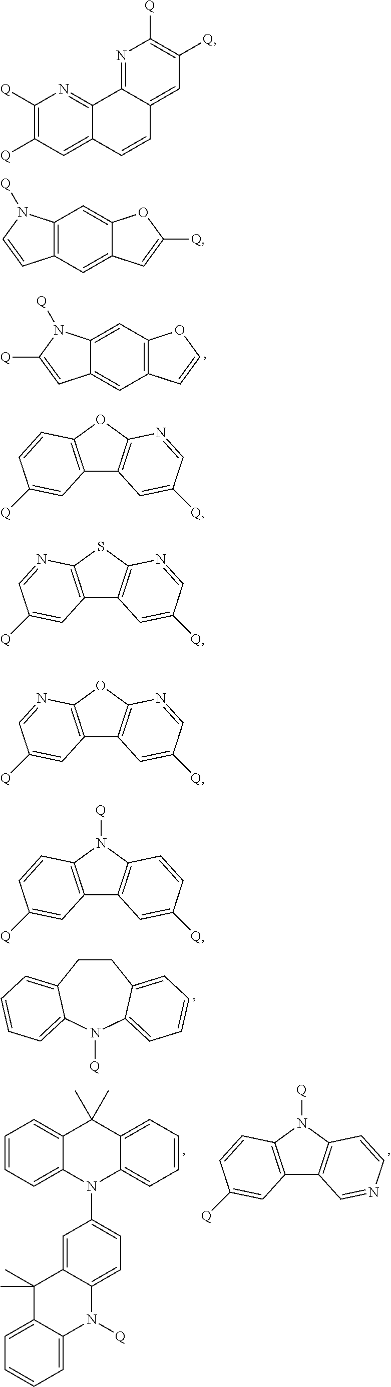

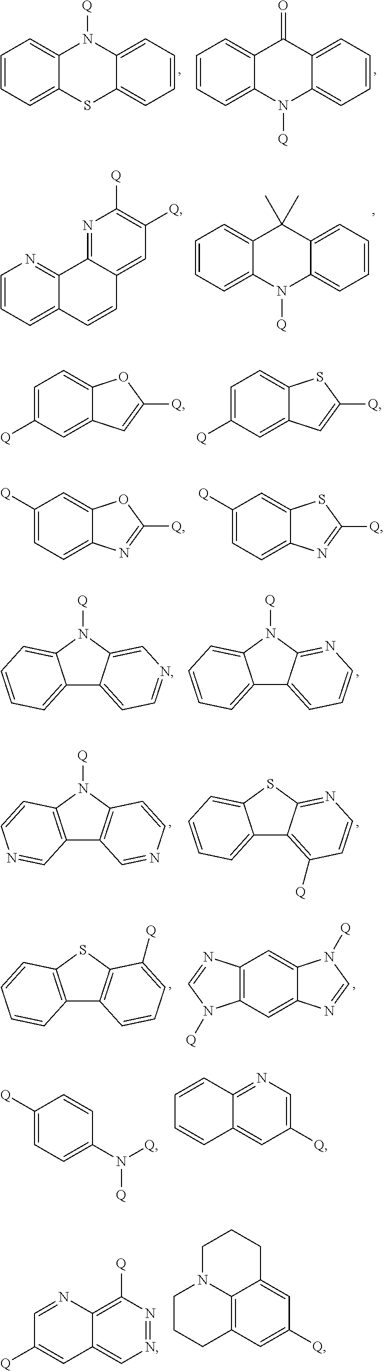

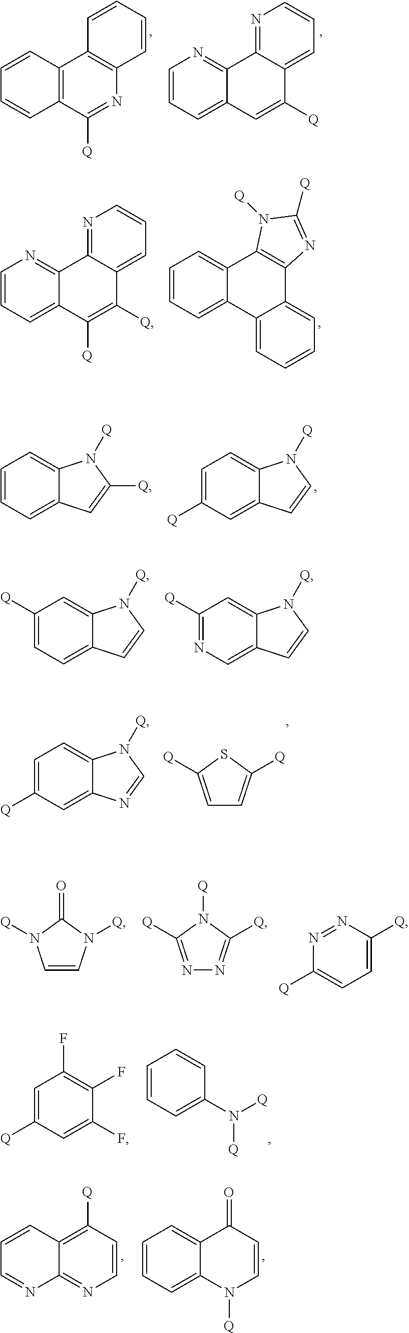

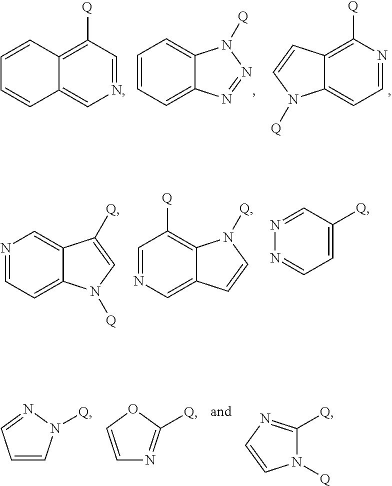

wherein, within each molecule:

Q is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂, and wherein the moieties A and B are defined above with respect to the first, second, and third aspects of the present invention.

In an example embodiment of the seventh and eighth aspects of the present invention, the moiety D, for each occurrence independently, can also be selected from List D6.

### List D6

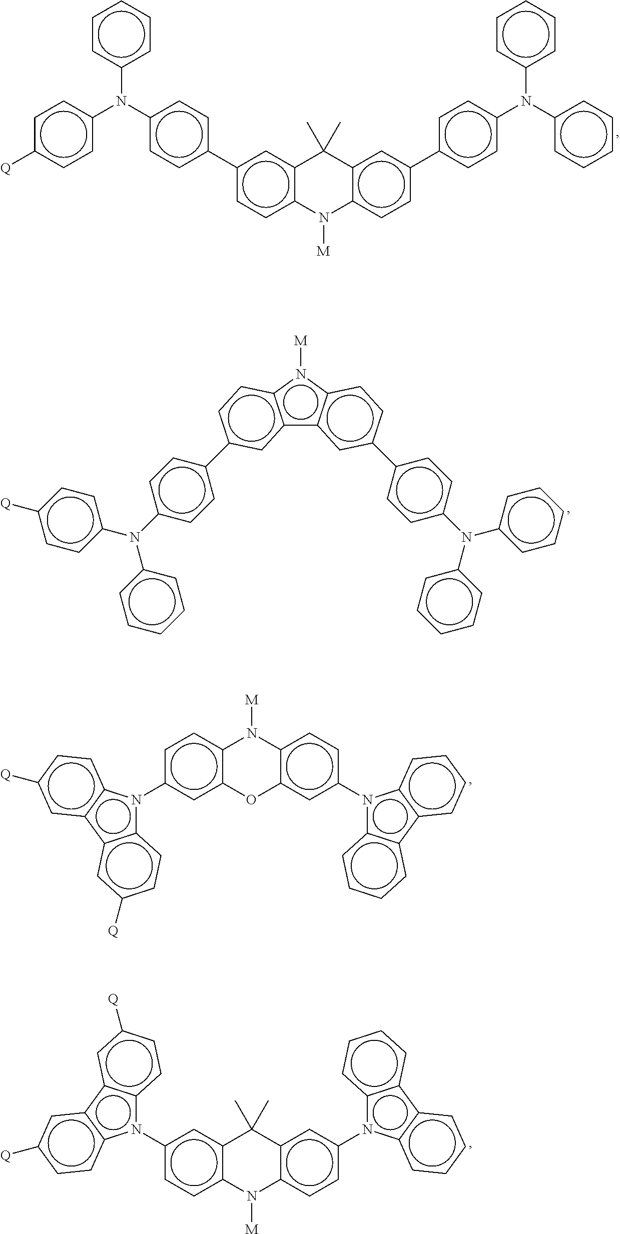

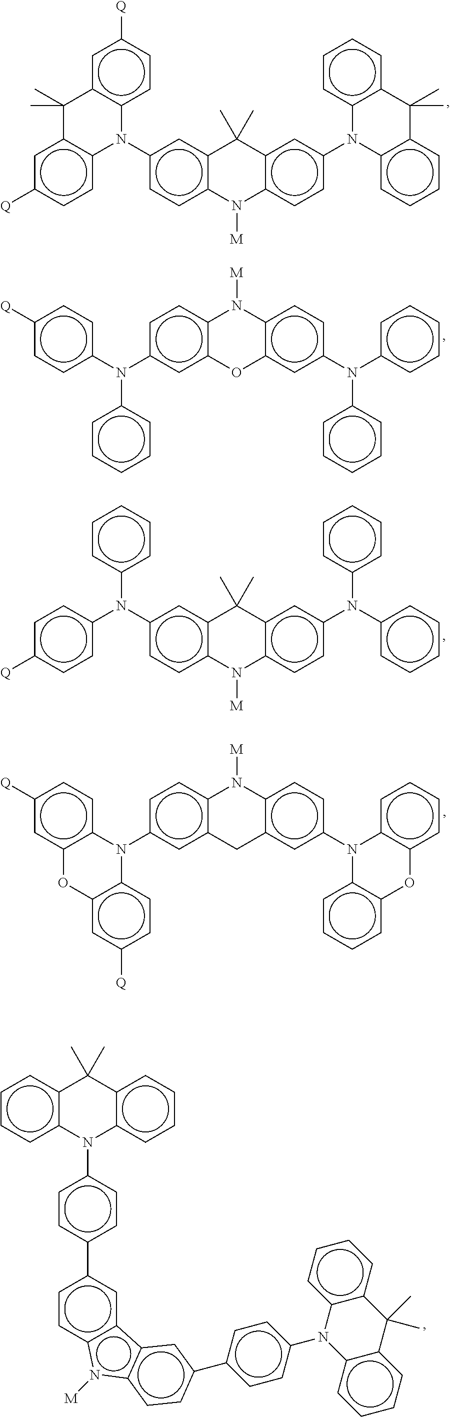

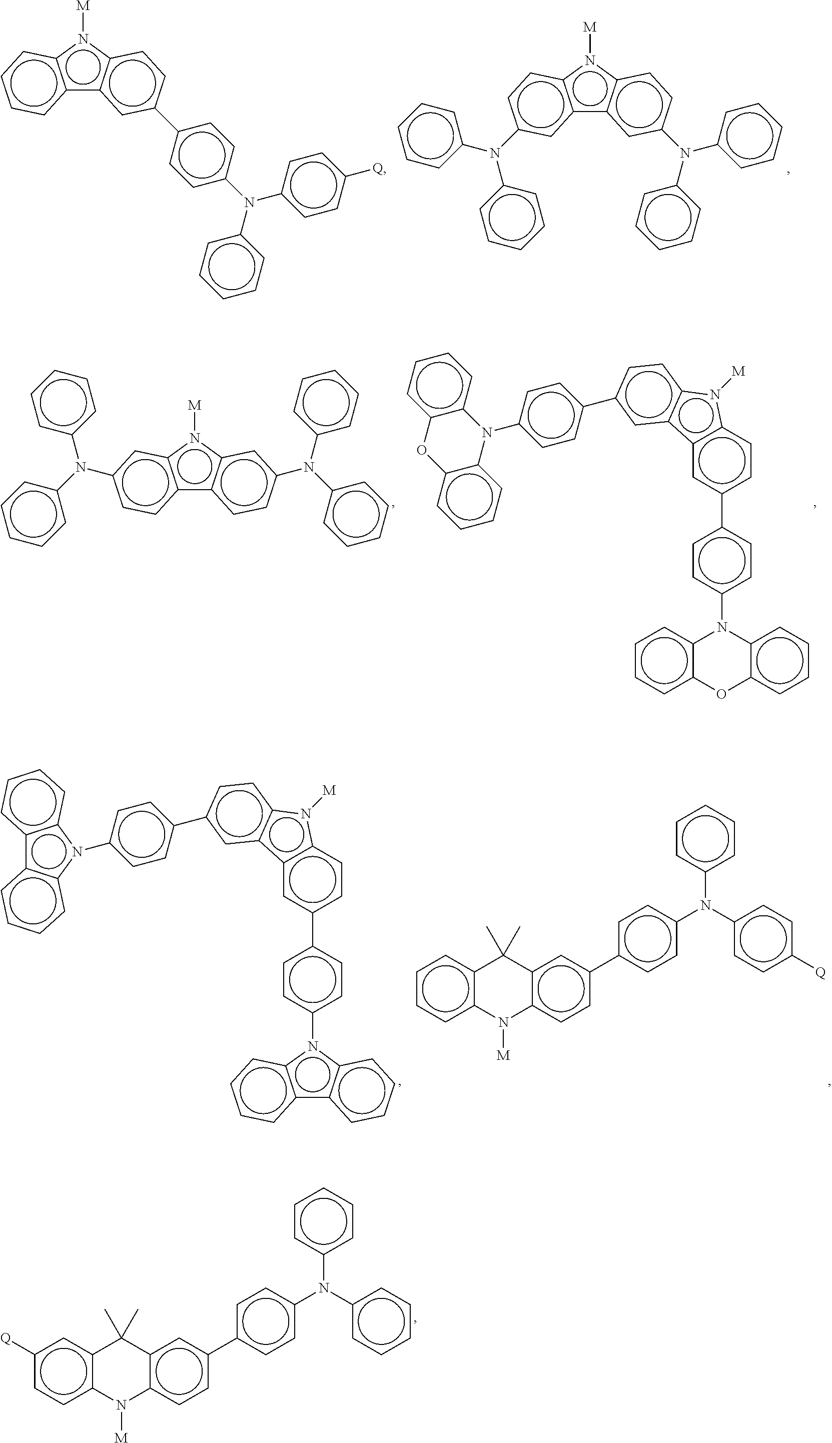

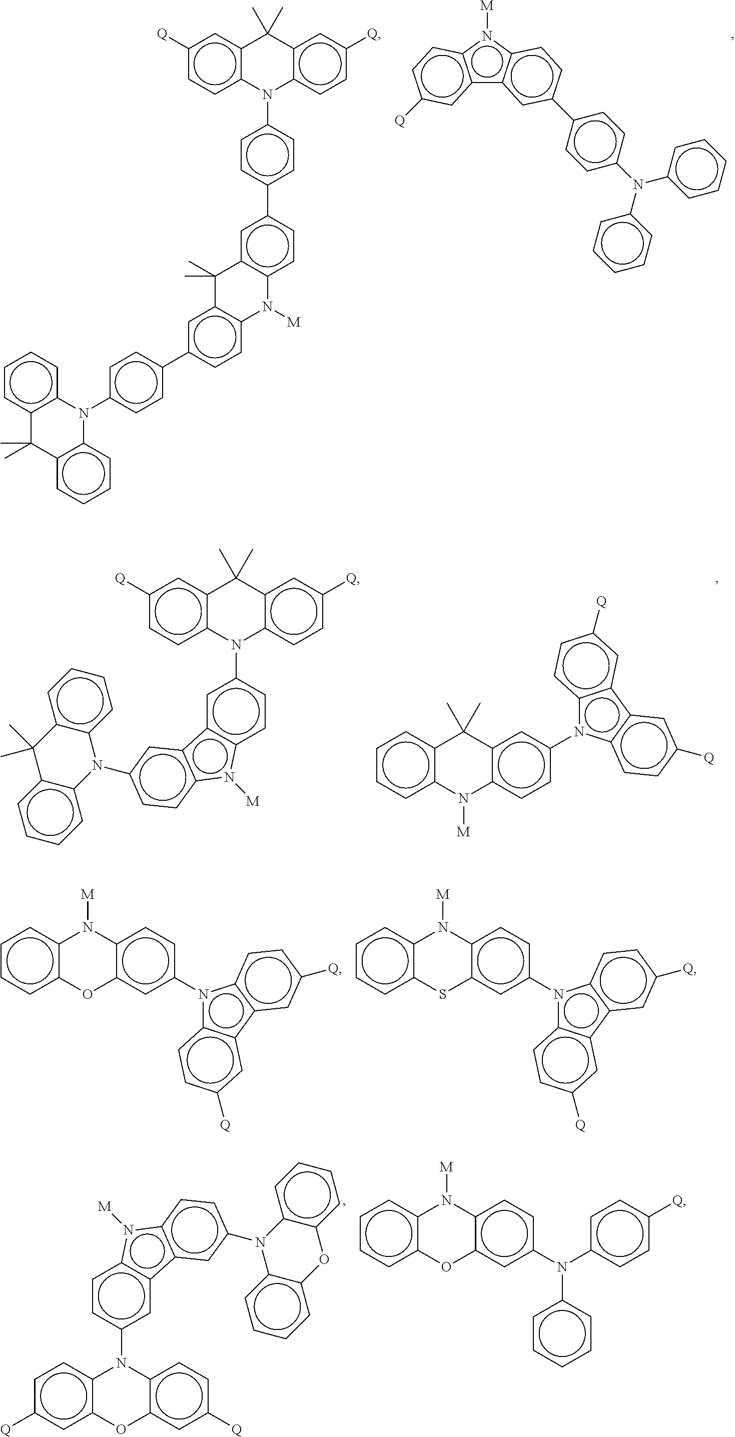

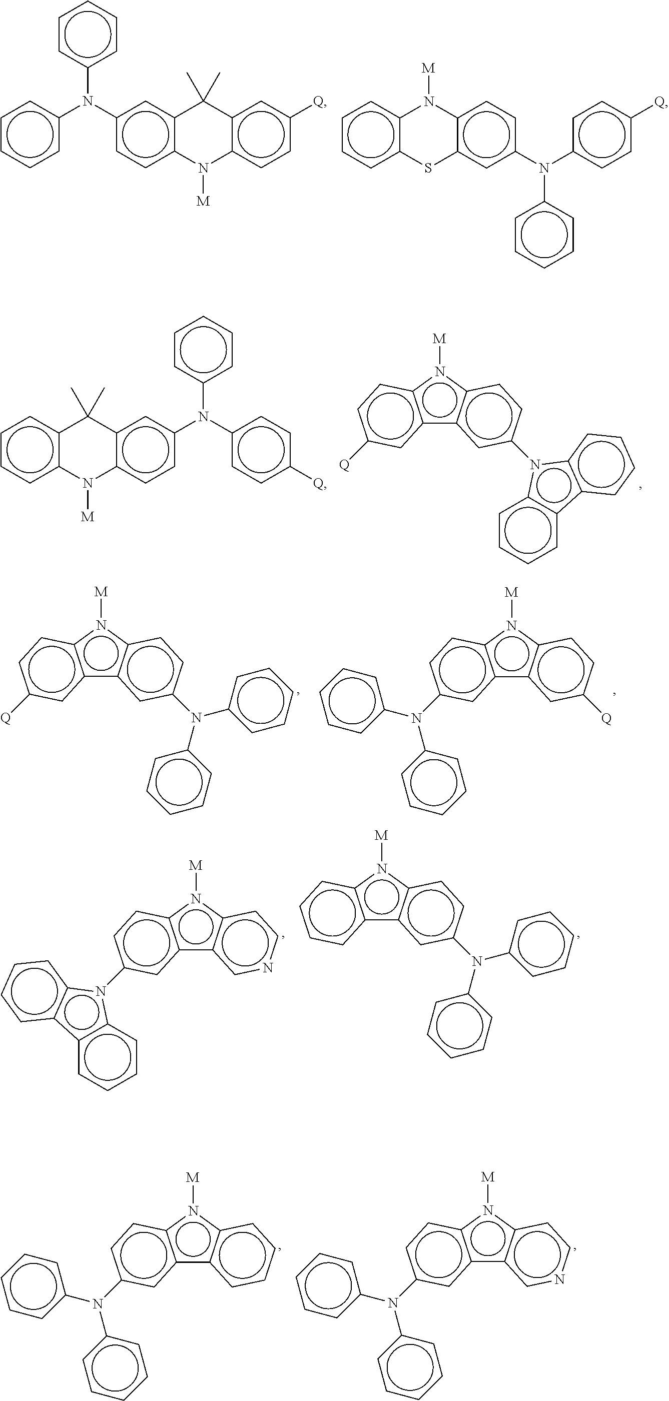

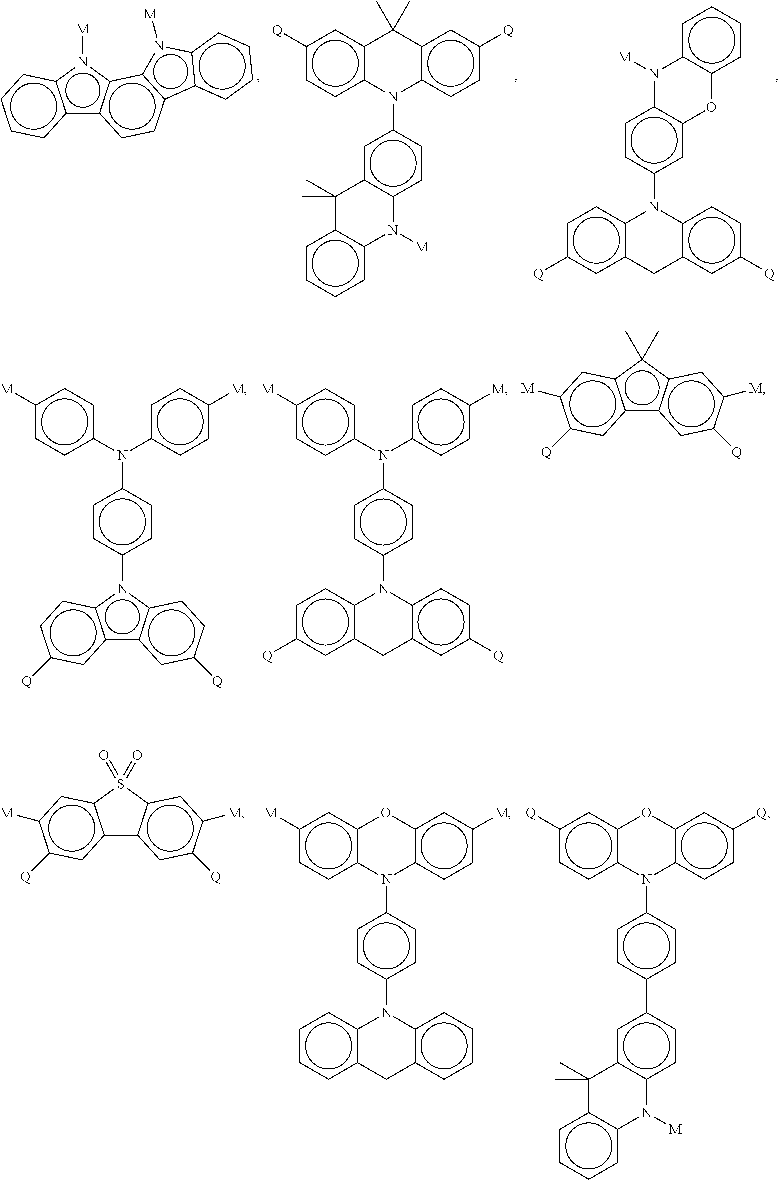

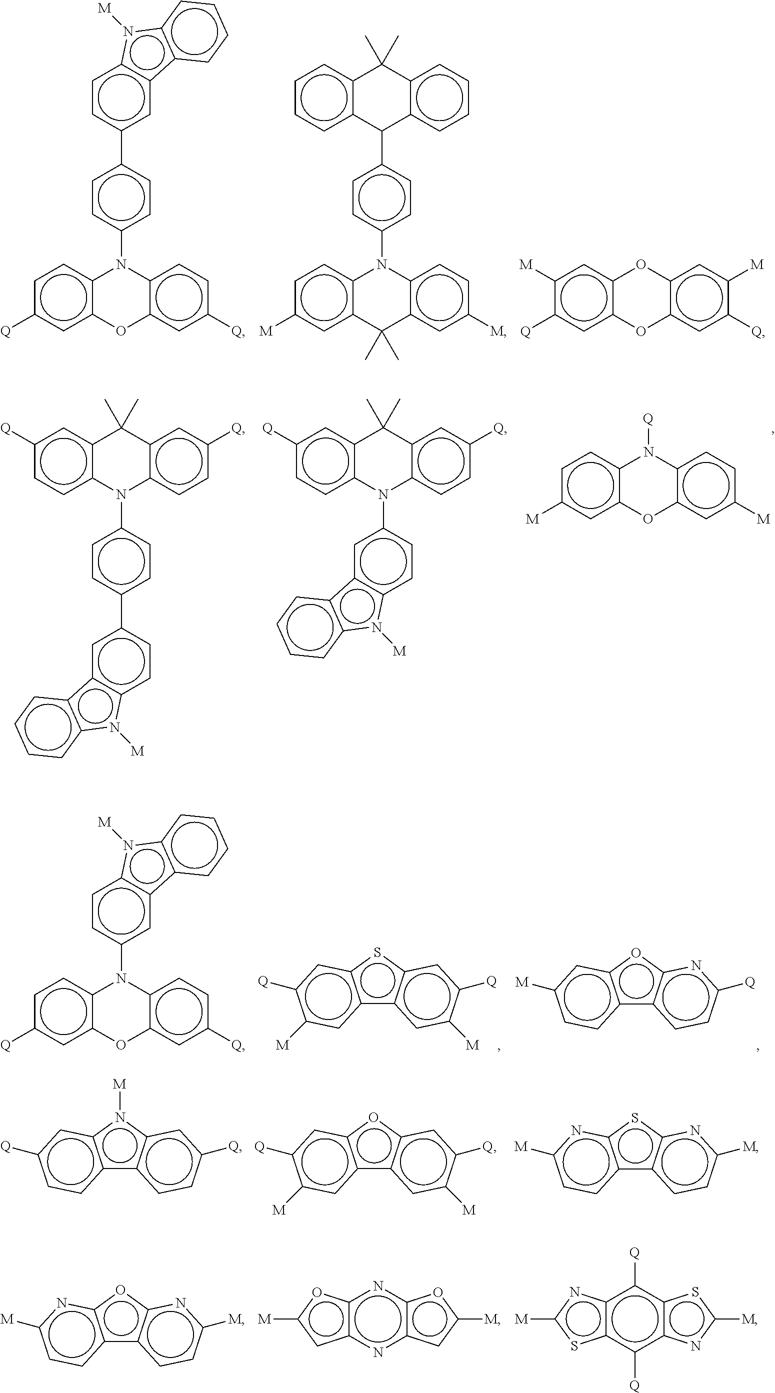

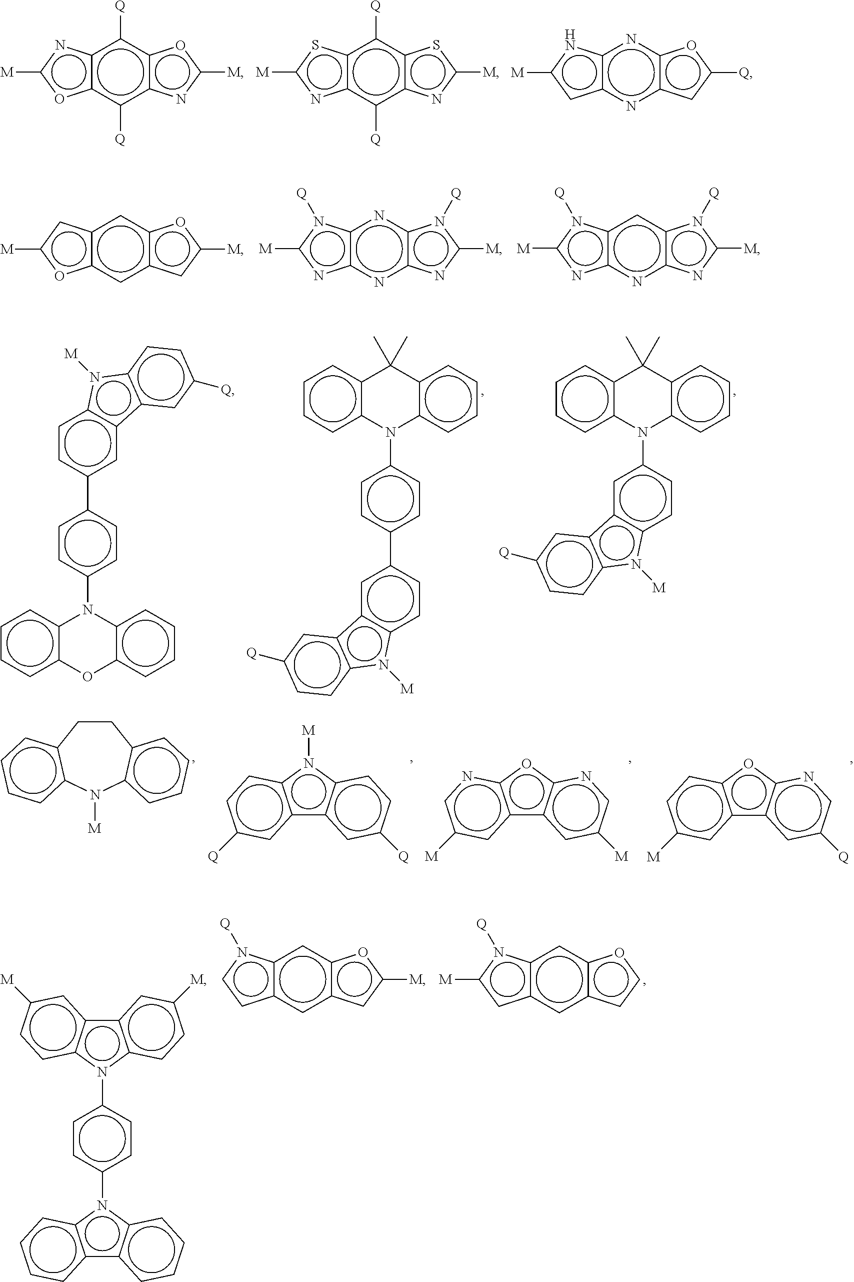

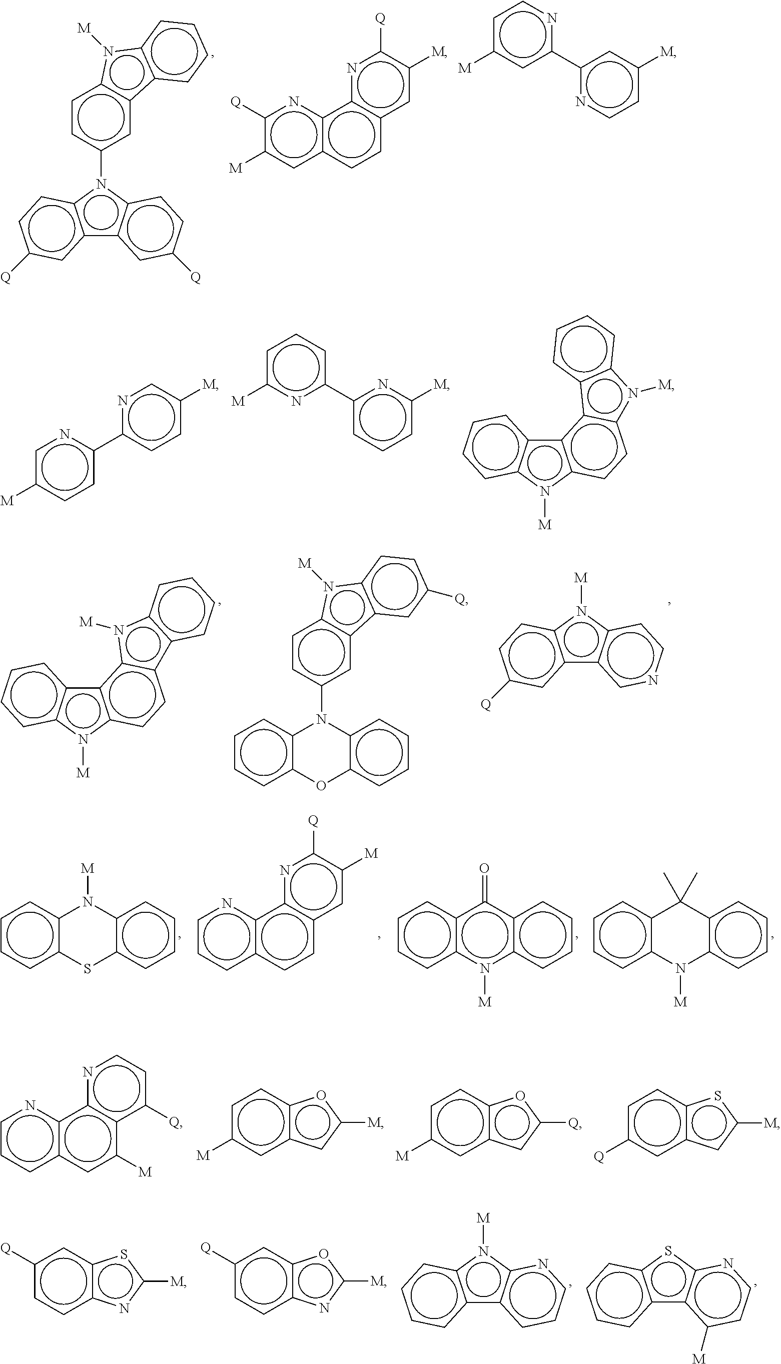

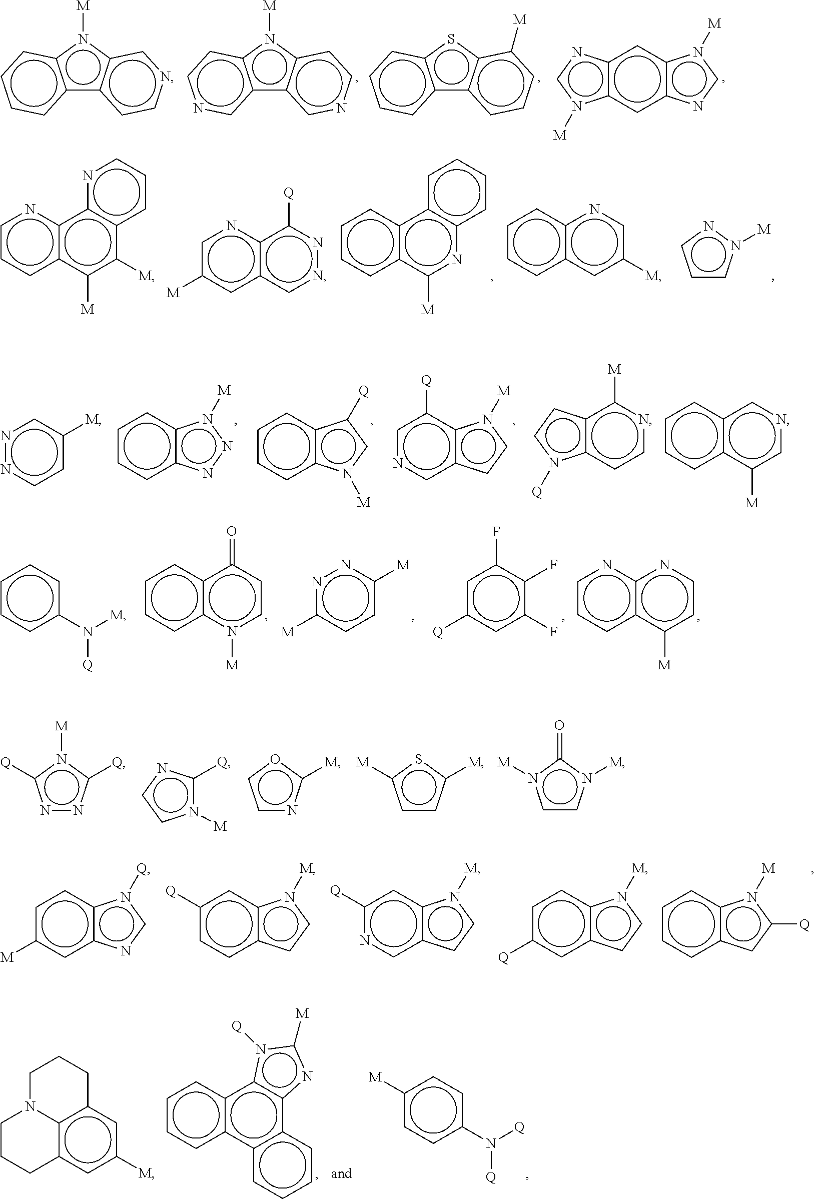

wherein, within each molecule:

Q is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂,

M is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂,

at least one of Q and M is the moiety B₀₋₂-A,

all groups Q are the same and all groups M are the same, and

each group Q is the same or different from any group M, and wherein the moieties A and B are defined above with respect to the first, second, and third aspects of the present invention.

In an example embodiment of the seventh and eighth aspects of the present invention, the moiety D, for each occurrence independently, can also be selected from List DN2.

### List DN2

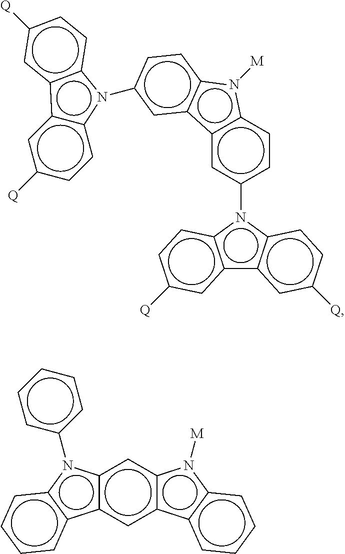

wherein, within each molecule:

Q is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂,

M is independently selected from the group consisting of the moiety A, a moiety B₀₋₂-A, H, C₁-C₃ alkyl, C₆-C₁₈ aryl, oxo, (5-20 atom) heteroaryl, and —N(C₆-C₁₈ aryl)₂,

at least one of Q and M is the moiety B₀₋₂-A,

all groups Q are the same and all groups M are the same, and each group Q is the same or different from any group M, and wherein the moieties A and B are defined above with respect to the first, second, and third aspects of the present invention.

In an example embodiment of the ninth aspect of the present invention, the moiety A, for each occurrence independently, is selected from List A4.

### List A4

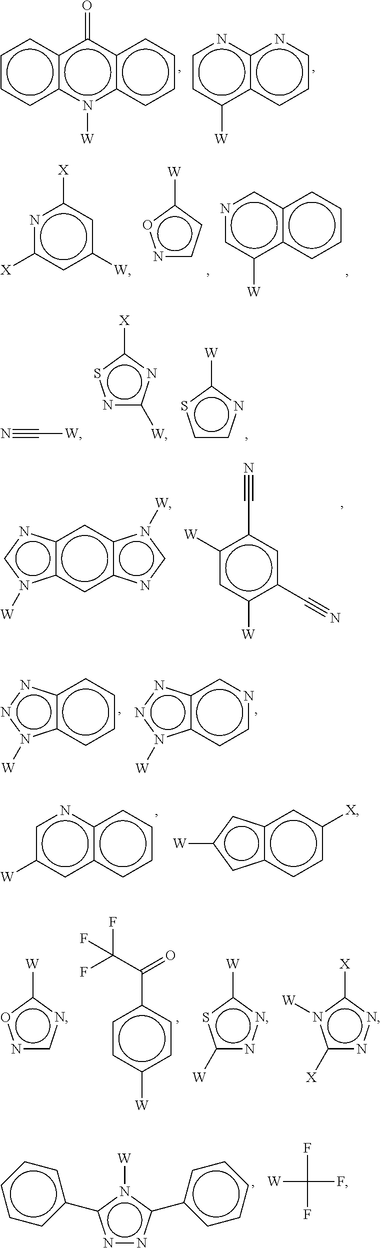

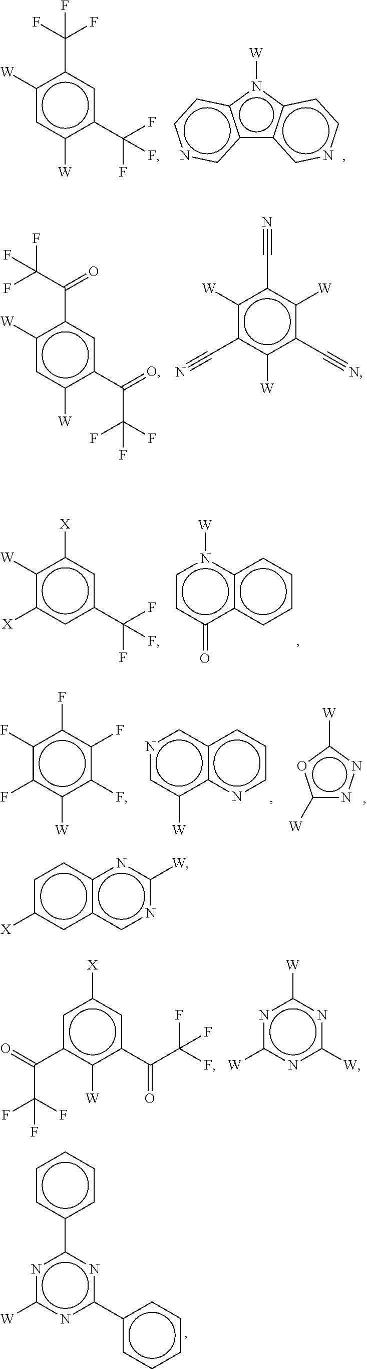

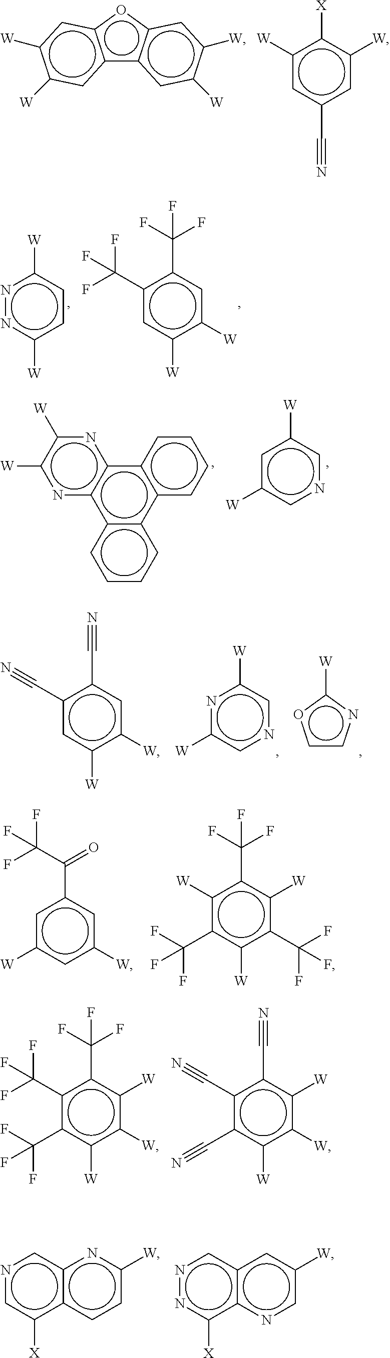

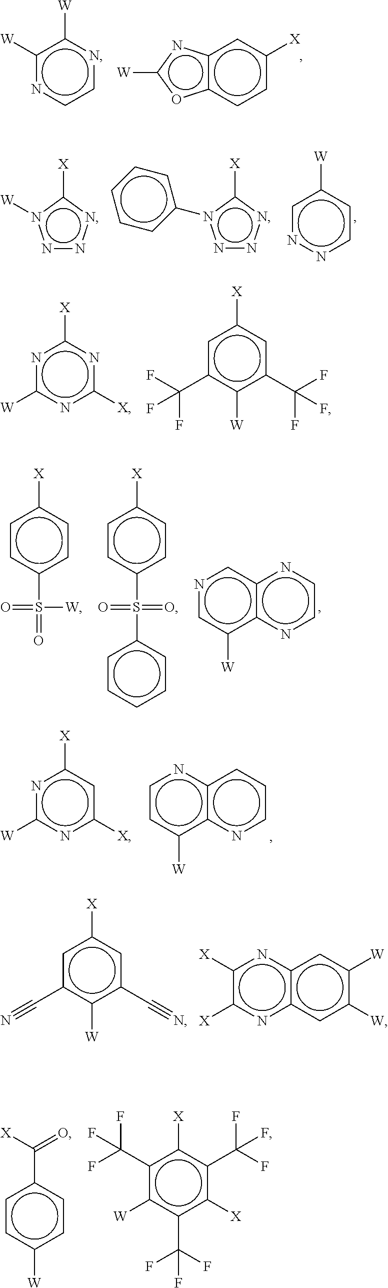

wherein, within each molecule:

W is the moiety D or a moiety B₀₋₂-D and each X is the moiety D or the moiety B₀₋₂-D,

all groups W are the same and all groups X are the same, and

each group W is the same or different from any group X, and wherein the moieties D and B are defined above with respect to the first, second, and third aspects of the present invention.

In an example embodiment of the tenth aspect of the present invention, the moiety A, for each occurrence independently, can be selected from List A4, List A5, or both.

### List A5

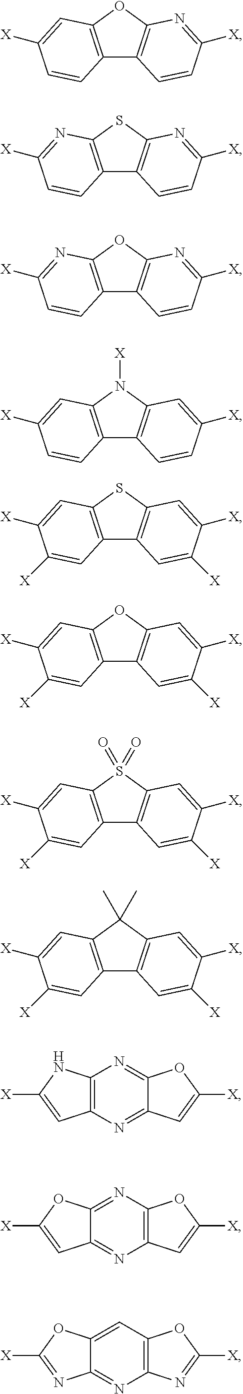

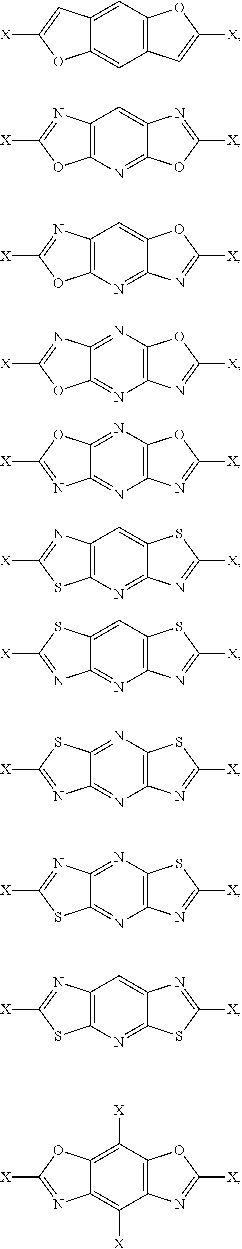

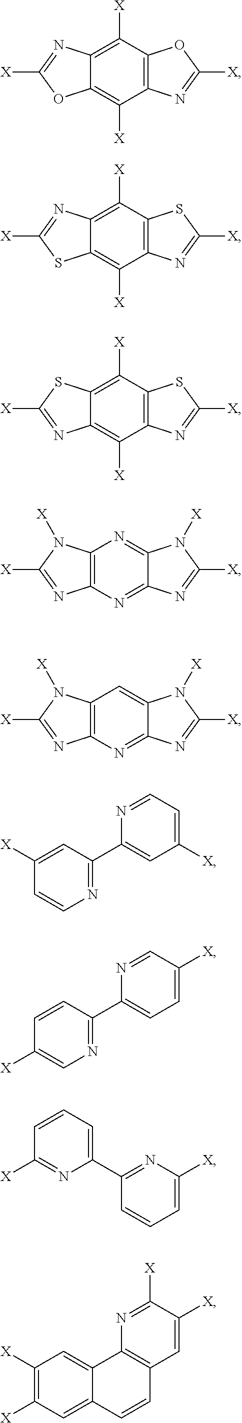

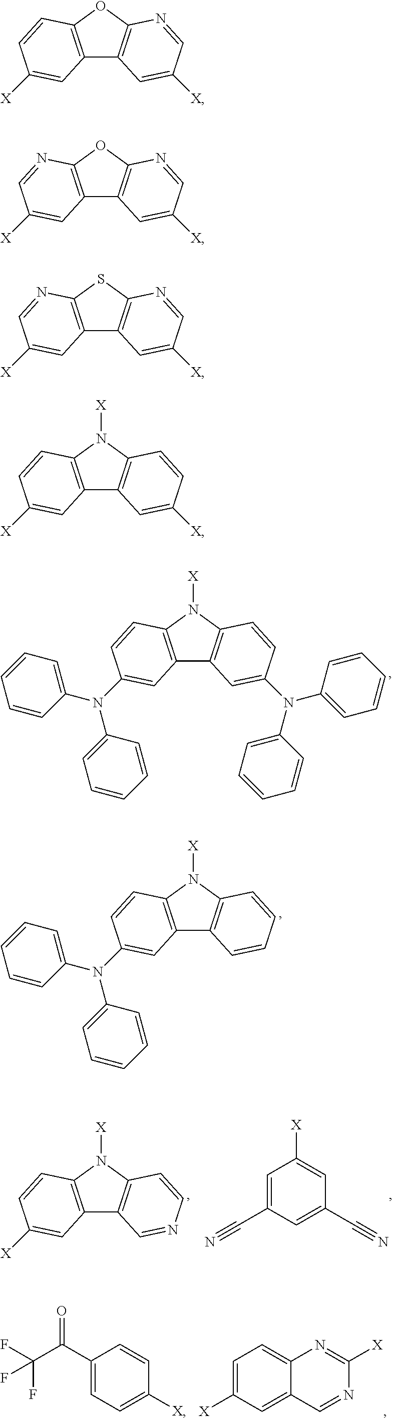

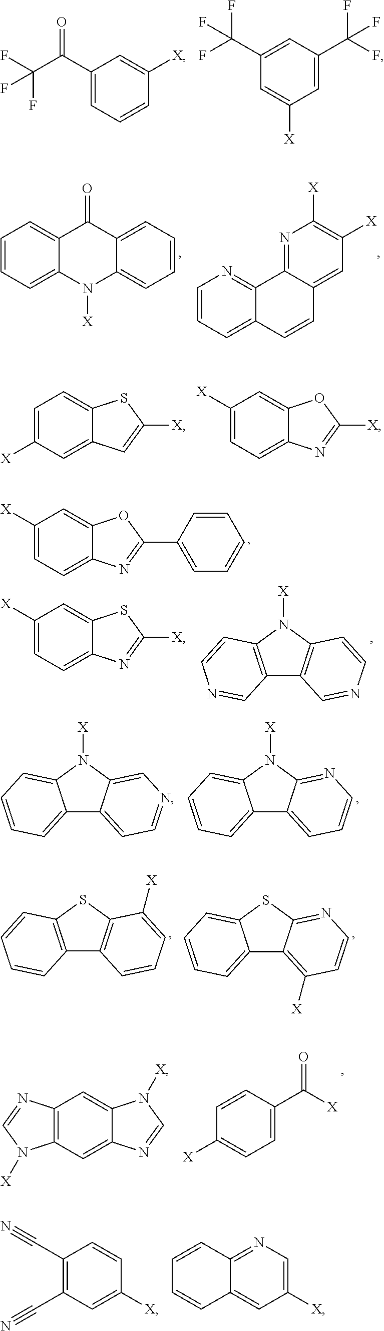

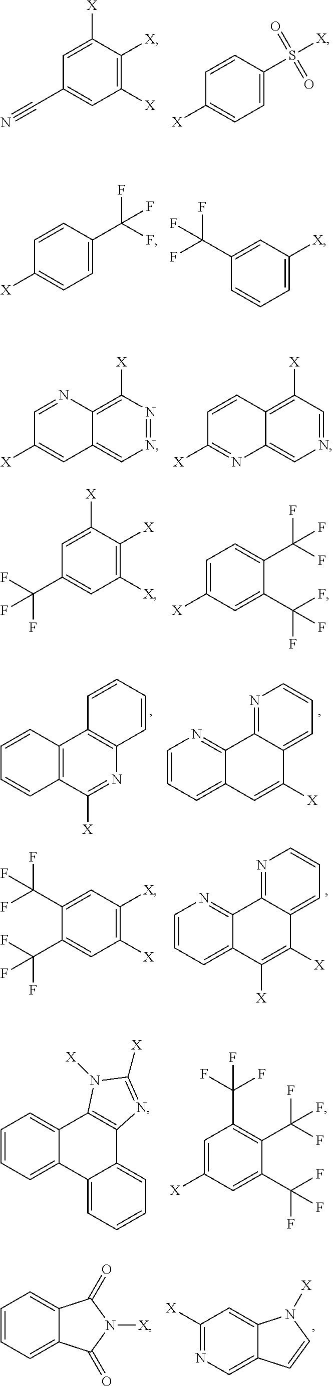

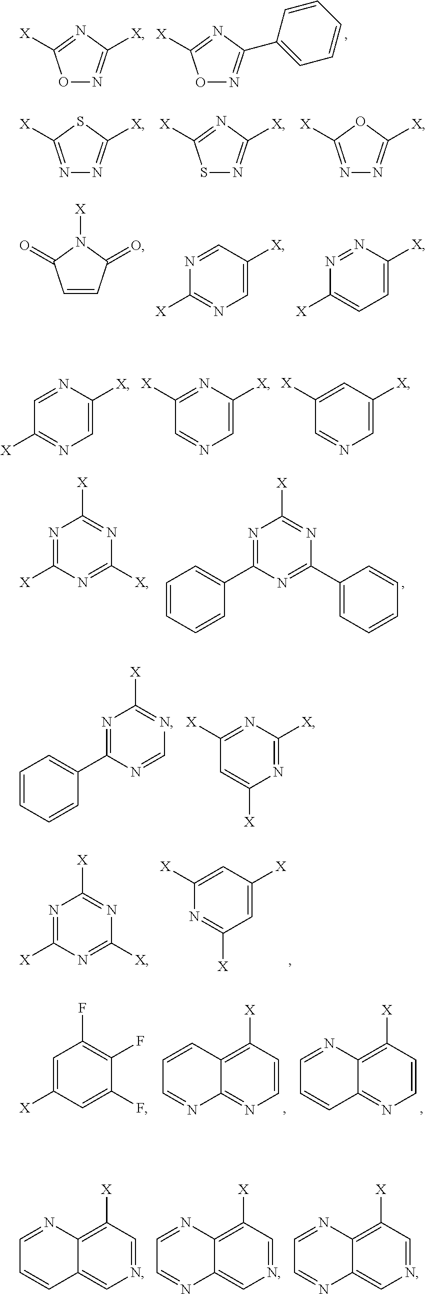

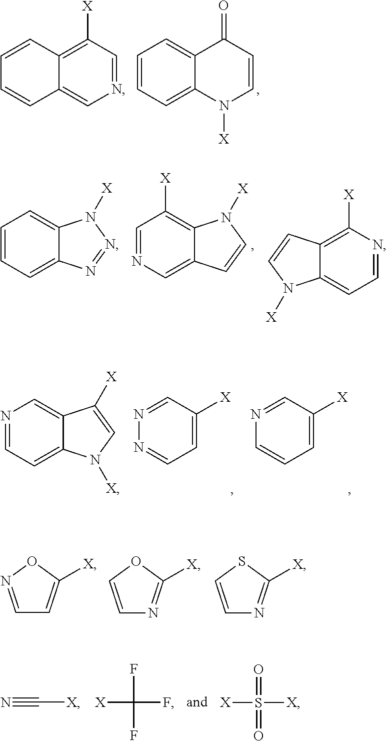

wherein, within each molecule:

X is selected from the group consisting of the moiety D, a moiety B₀₋₂-D, H, C₁-C₃ alkyl, C₆-C₁₈ aryl , oxo, C₁-C₃ haloalkyl, —CN, —CF₃, —C(O)C₁-C₃ haloalkyl, —F, and —S(O₂)H, and wherein the moieties D and B are defined above with respect to the first, second, and third aspects of the present invention.

In an example embodiment of the tenth and eleventh aspects of the present invention, the moiety A, for each occurrence independently, can be selected from List A4, List A5, List A6, or any combination thereof.

### List A6

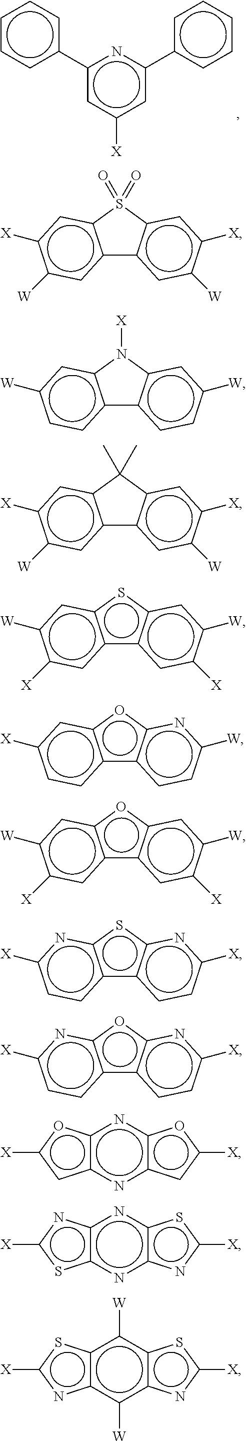

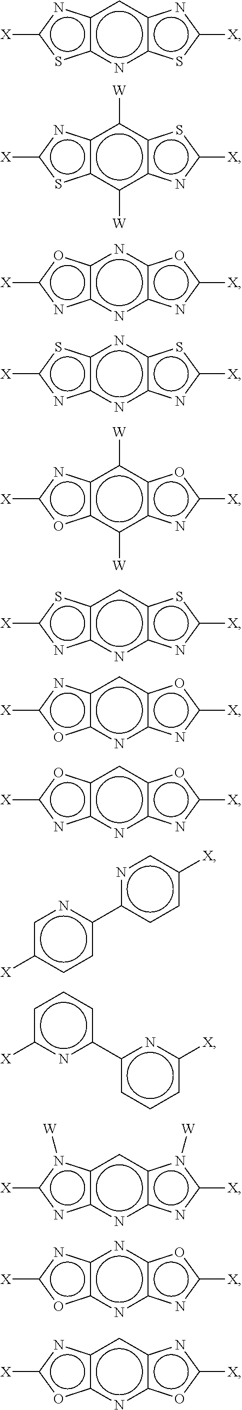

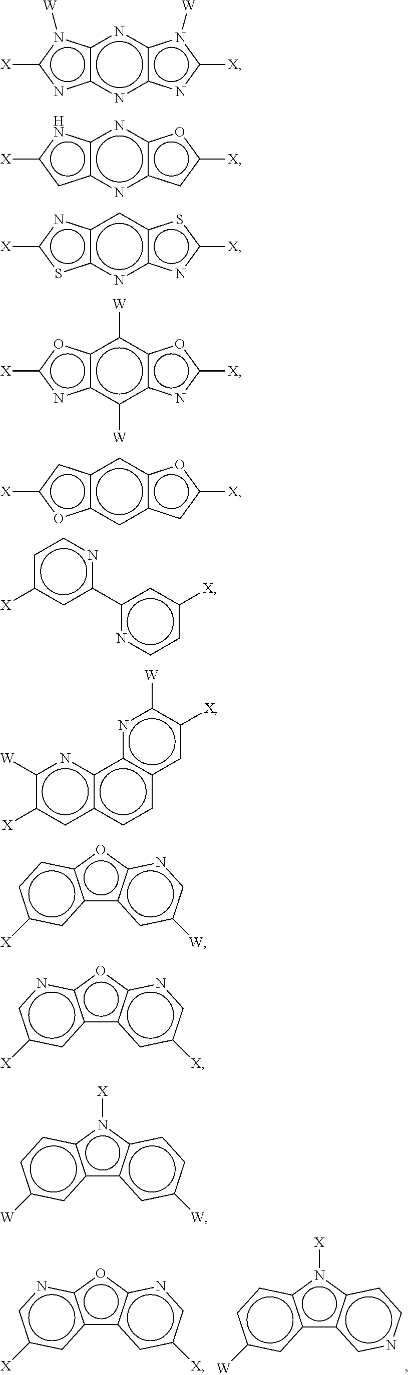

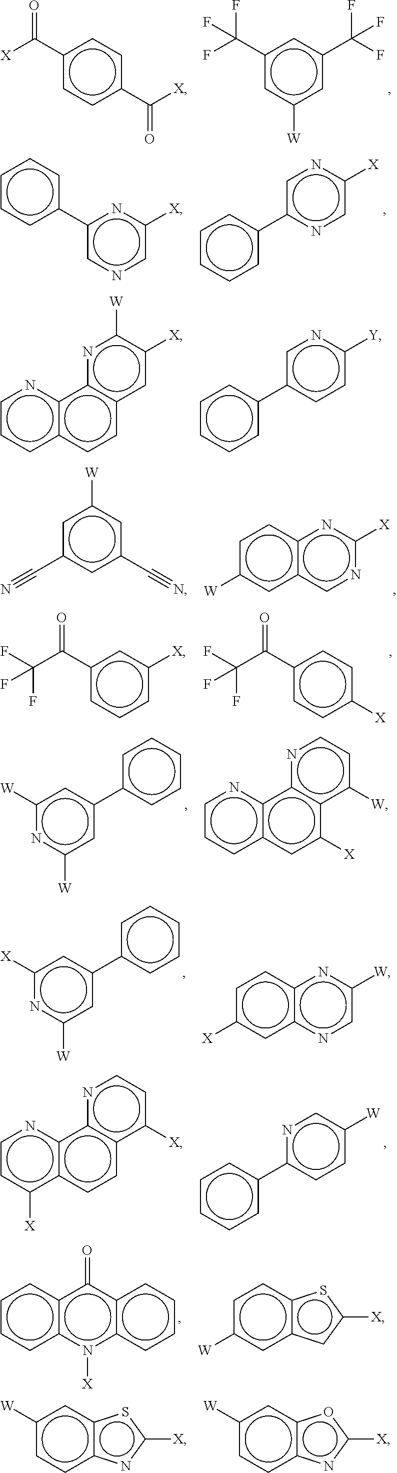

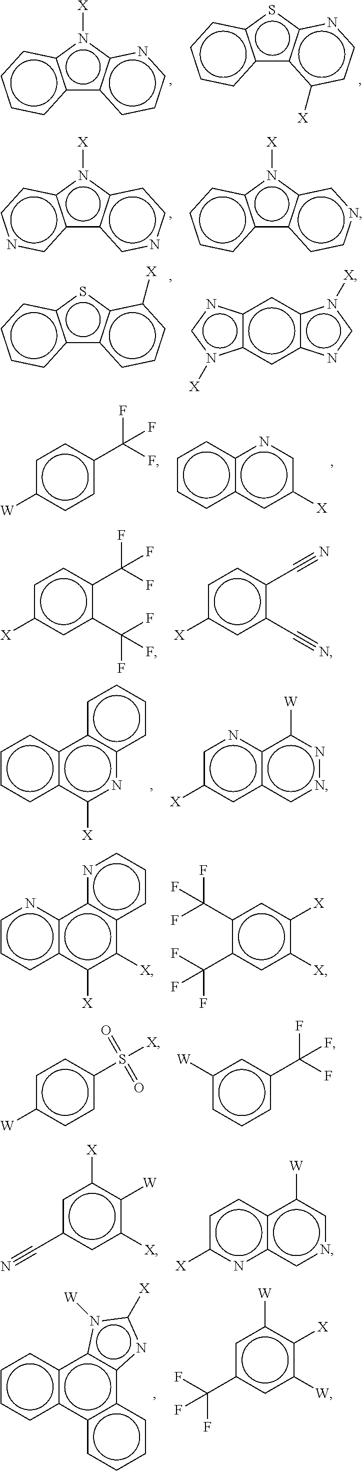

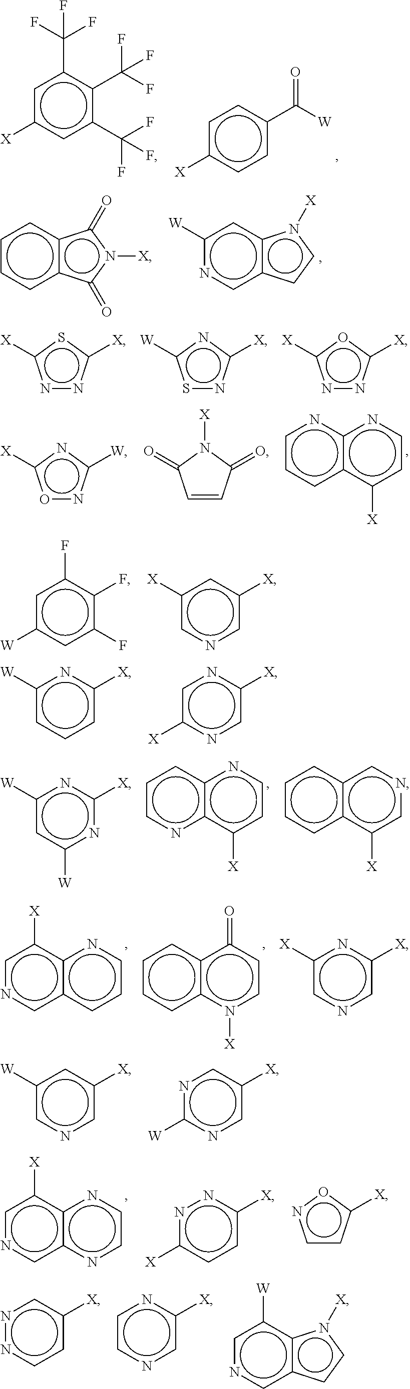

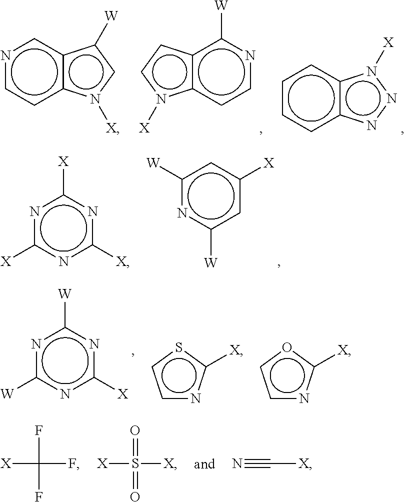

wherein, within each molecule:

X is selected from the group consisting of a moiety B₀₋₂-D, H, C₁-C₃ alkyl, C₆-C₁₈ aryl, oxo, C₁-C₃ haloalkyl, —CN, —CF₃, —C(O)C₁-C₃ haloalkyl, —F, and —S(O₂)H,

W is selected from the group consisting of the moiety B₀₋₂-D, H, C₁-C₃ alkyl, C₁-C₃ acylalkyl, C₆-C₁₈ aryl, oxo, C₁-C₃ haloalkyl, —CN, —CF₃, —C(O)C₁-C₃ haloalkyl, —F, and —S(O₂)H,

at least one of W and X is the moiety B₀₋₂-D,

all groups W are the same and all groups X are the same, and

each group W is the same or different from any group X, and wherein the moieties D and B are defined above with respect to the first, second, and third aspects of the present invention.

In an example embodiment of the tenth and eleventh aspects of the present invention, the moiety A, for each occurrence independently, can be selected from List A4, List A5, List A6, List AN2, or any combination thereof. In certain embodiments, at least one occurrence of the moiety A is selected from List AN2. In certain embodiments, each occurrence of the moiety A is independently selected from List AN2.

### List AN2

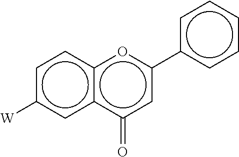

wherein, within each molecule:

W is the moiety D or a moiety B₀₋₂-D and wherein the moieties D and B are defined above with respect to the first, second, and third aspects of the present invention.

In an example embodiment of the twelfth aspect of the present invention, the moiety B, for each occurrence independently, is selected from List B3.

### List B3

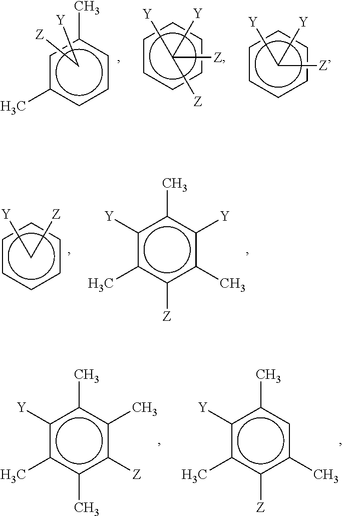

wherein, within each molecule:

Y is the moiety A, the moiety B₀₋₁-A, the moiety D, or the moiety B₀₋₁-D and each Z is the moiety A, a moiety B₀₋₁-A, the moiety D, or a moiety B₀₋₁-D,

within a given molecule all groups Y are the same and all groups Z are the same, and

each group Y is the same or different from any group Z, and wherein the moieties A and D are defined above with respect to the first, second, and third aspects of the present invention.

In an example embodiment of the thirteenth aspect of the present invention, the moiety B, can also be selected from List B3, List B4, or both.

### List B4

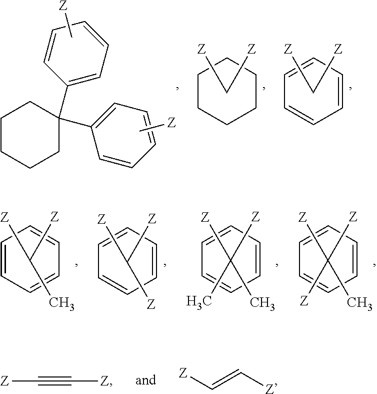

wherein, within each molecule:

Z is independently selected from the group consisting of the moiety A, a moiety B₀₋₁-A, the moiety D, a moiety B₀₋₁-D, H, C₁-C₃ alkyl, and C₆-C₁₈ aryl, and wherein the moieties A and D are defined above with respect to the first, second, and third aspects of the present invention.

In an example embodiment of the twelfth and thirteenth aspects of the present invention, the moiety B, can also be selected from List B3, List B4, List B5, or any combination thereof.

### List B5

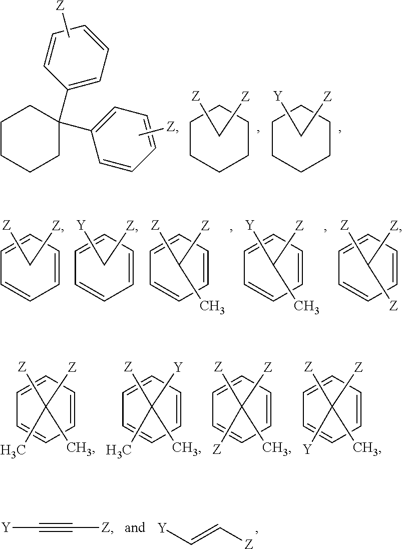

wherein, within each molecule:

Z is the moiety A, a moiety B₀₋₁-A, the moiety D, a moiety B₀₋₁-D, H, C₁-C₃ alkyl, or C₆-C₁₈ aryl,

Y is the moiety A, the moiety B₀₋₁-A, the moiety D, or the moiety B₀₋₁-D and each Z is the moiety A, a moiety B₀₋₁-A, the moiety D, or a moiety B₀₋₁-D,

within a given molecule all groups Y are the same and all groups Z are the same, and

each group Y is the same or different from any group Z, and wherein the moieties A and D are defined above with respect to the first, second, and third aspects of the present invention.

In an example embodiment of the twelfth aspect of the present invention, the moiety B, for each occurrence independently, is selected from List B3, List B4, List B5, List B6, or any combination thereof.

List B6

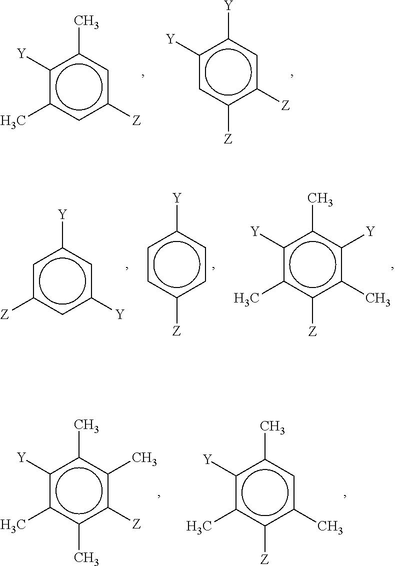

wherein, within each molecule:

Y is the moiety A, the moiety B₀₋₁-A, the moiety D, or the moiety B₀₋₁-D and each Z is the moiety A, a moiety B₀₋₁-A, the moiety D, or a moiety B₀₋₁-D,

within a given molecule all groups Y are the same and all groups Z are the same, and

each group Y is the same or different from any group Z, and wherein the moieties A and D are defined above with respect to the first, second, and third aspects of the present invention.

In an example embodiment of the thirteenth aspect of the present invention, the moiety B, for each occurrence independently, is selected from List B3, List B4, List B5, List B6, List B7, or any combination thereof.

### List B7

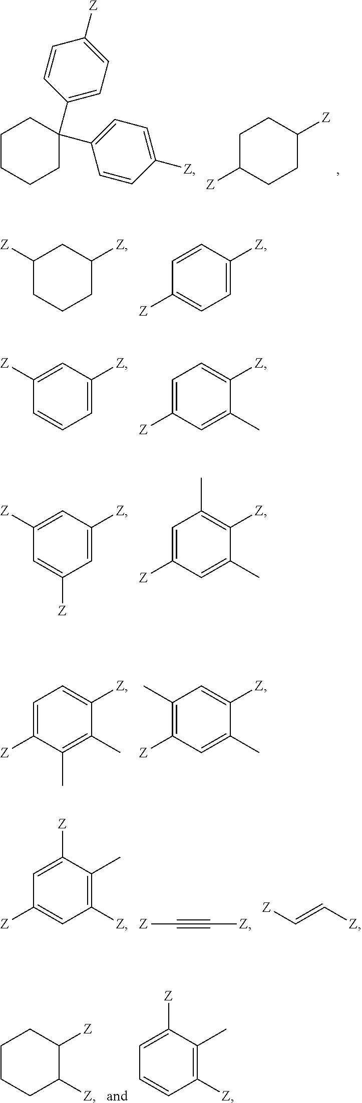

wherein, within each molecule:

Z is the moiety A, the moiety B₀₋₁-A, the moiety D, the moiety B₀₋₁-D, H, C₁-C₃ alkyl, or C₆-C₁₈ aryl, and wherein the moieties A and D are defined above with respect to the first, second, and third aspects of the present invention.

In an example embodiment of the twelfth and thirteenth aspects of the present invention, the moiety B, for each occurrence independently, is selected from List B3, List B4, List B5, List B6, List B7, List B8 or any combination thereof.

### List B8

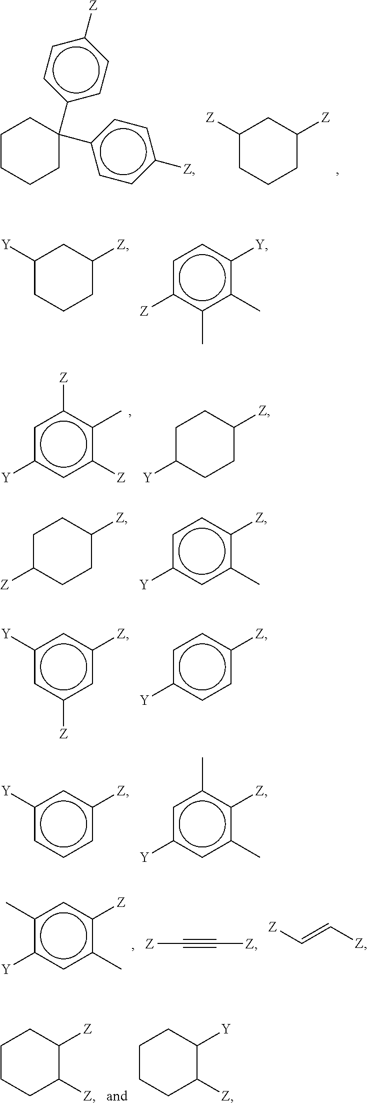

wherein, within each molecule:

Z is the moiety A, the moiety B₀₋₁-A, the moiety D, the moiety B₀₋₁-D, H, C₁-C₃ alkyl, or C₆-C₁₈ aryl,

Y is the moiety A, the moiety B₀₋₁-A, the moiety D, the moiety B₀₋₁-D, H, C₁-C₃ alkyl, or C₆-C₁₈ aryl,

within a given molecule all groups Y are the same and all groups Z are the same, and

each group Y is the same or different from any group Z, and wherein the moieties A and D are defined above with respect to the first, second, and third aspects of the present invention.

In an example embodiment of any one of the first through thirteenth aspects of the present invention described above, the moiety D is optionally substituted with one or more substituents each independently selected from C₁-C₃ alkyl, C₆-C₁₈ aryl, or oxo, and wherein A, B, and D are defined above with respect to the first or second aspects of the present invention.

In an example embodiment of any one of the first through thirteenth aspects of the present invention described above, the moiety D is optionally substituted with one or more substituents each independently selected from (5-20 atom) heteroaryl or —N(C₆-C₁₈aryl)₂, and wherein A, B, and D are defined above with respect to the first or second aspects of the present invention.

In an example embodiment of any one of the first through thirteenth aspects of the present invention described above, the moiety D is optionally substituted with one or more substituents each independently selected from C₁-C₃ alkyl, C₆-C₁₈ aryl, oxo, (5-20 atom) heteroaryl, or —N(C₆-C₁₈aryl)₂, and wherein A, B, and D are defined above with respect to the first or second aspects of the present invention.

In an example embodiment of any one of the first through thirteenth aspects of the present invention described above, the moiety A is optionally substituted with one or more substituents each independently selected from C₁-C₃ alkyl, C₆-C₁₈ aryl, oxo, C₁-C₃ haloalkyl, —CN, —CF₃, —C(O)C₁-C₃ haloalkyl, —F, and —S(O₂)H, and wherein A, B, and D are defined above with respect to the first or second aspects of the present invention.

In an example embodiment of any one of the first through thirteenth aspects of the present invention described above, the moiety B is optionally substituted with C₁-C₃ alkyl, and wherein A, B, and D are defined above with respect to the first or second aspects of the present invention.

In an example embodiment of any one of the first through thirteenth aspects of the present invention described above, the moiety B is optionally substituted with C₆-C₁₈ aryl, and wherein A, B, and D are defined above with respect to the first or second aspects of the present invention.

In an example embodiment of any one of the first through thirteenth aspects of the present invention described above, the moiety B is optionally substituted with one or more substituents each independently selected from C₁-C₃ alkyl or C₆-C₁₈ aryl, and wherein A, B, and D are defined above with respect to the first or second aspects of the present invention.

In a fourteenth aspect, the present invention is a molecule of any one of the structural formulas represented in Tables M, N, O, Q, B, or R and is optionally substituted.

According to certain embodiments of the fourteenth aspect, the present invention is a molecule represented by any one structural formula in Tables M, N, O, Q, B, or R and is optionally substituted. According to certain embodiments, the present invention is a molecule represented by any one structural formula in Tables M, N, O, Q, or R and is optionally substituted. According to certain embodiments, the present invention is a molecule represented by any one structural formula in Tables N′, N″, N′″, Q′, or R′ and is optionally substituted. The variables and substitution patterns on the molecule may be selected as described below with respect to the fourteenth aspect.

According to certain embodiments of the fourteenth aspect, the present invention is a molecule represented by any one structural formula as shown in Table M, N, N′, N″, N′″, O, Q, Q′, B, R, or R′, wherein

at each substitutable carbon independently, the molecule is optionally substituted with R^{C};

R^{C} is selected from a C₁-C₆ alkyl, a C₃-C₆ cycloalkyl, —OH, —CN, a halo, a C₆-C₁₂ aryl, a 5-20 atom heteroaryl, or —N(R¹⁹)₂; and

each R¹⁹, independently, is H, a C₁-C₆ alkyl, a C₅-C₁₂ cycloalkyl, or a C₆-C₁₈ aryl.

According to certain embodiments of the fourteenth aspect, the molecule is represented by one of the structural formulas in Table M, N, N′, N″, N′″, O, Q, Q′, B, R, or R′, and is optionally substituted at any substitutable carbon with R^{C}. According to certain embodiments, no atom is substituted. According to certain embodiments, at least one substitutable carbon is substituted with R^{C}. The variables and base molecules may be selected as described above or below with respect to the fourteenth aspect.

According to certain embodiments of the fourteenth aspect, the molecule is represented by any one compound in Tables M, N, O, Q, B, R, N′, N″, N′″, Q′, or R′ wherein atoms marked with * are optionally substituted with R^{C}. According to certain embodiments, no atom is substituted with R^{C}. According to certain embodiments, at least one atom marked with * is substituted with R^{C}. According to certain embodiments, at least one atom marked with * is substituted with R^{C}, and any substitutable carbon is optionally substituted with R^{C}. According to certain embodiments, at least one atom marked with * is substituted with R^{C}, and no other atom is substituted. The variables and base molecules may be selected as described above or below with respect to the fourteenth aspect.

According to certain embodiments of the fourteenth aspect, each R^{C}, independently, is selected from a C₁-C₆ alkyl, a C₃-C₆ cycloalkyl, —OH, —CN, a halo, a C₆-C₁₂ aryl, a 5-20 atom heteroaryl, or —N(R¹⁹)₂. According to certain embodiments, each R^{C}, independently, is selected from a C₁-C₃ alkyl, a C₃-C₆ cycloalkyl, a C₆-C₁₀ aryl, a 5-20 atom heteroaryl, halo, or —CN. According to certain embodiments of the fourteenth aspect, each R^{C}, independently, is selected from methyl or phenyl. The remainder of the variables, base molecules, and substitution patterns, may be selected as described above or below with respect to the fourteenth aspect.

According to example embodiments of the fourteenth aspect, each R¹⁹, independently, is H, a C₁-C₆ alkyl, a C₅-C₁₂ cycloalkyl, or a C₆-C₁₈ aryl. According to certain embodiments, each R¹⁹, independently, is H, a C₁-C₃ alkyl, a C₃-C₆ cycloalkyl, or phenyl. According to certain embodiments, each R¹⁹ is phenyl. The remainder of the variables, base molecules, and substitution patterns, may be selected as described above or below with respect to the fourteenth aspect.

In an example embodiment of the fourteenth aspect, the present invention is any one molecule selected from compounds M1 to M53, listed in Table M. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE M

| | |
|---|---|
| 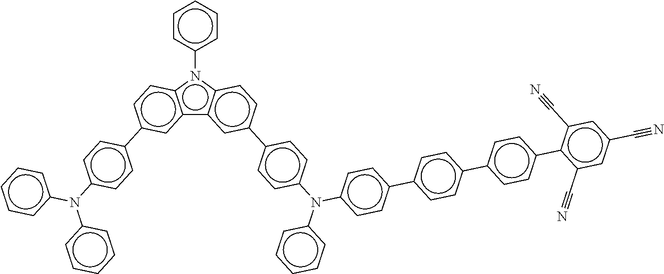 | M1 |
| 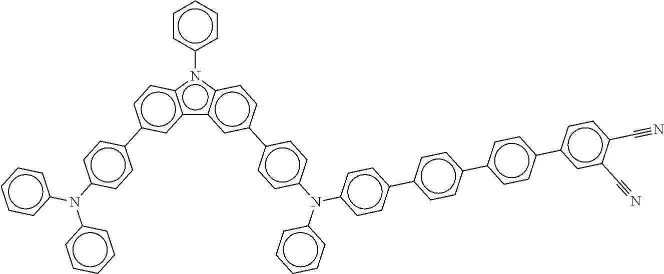 | M2 |
| 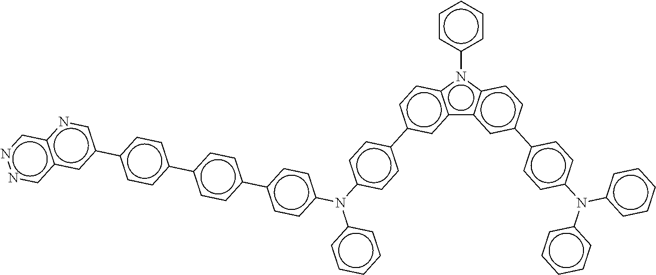 | M3 |
| 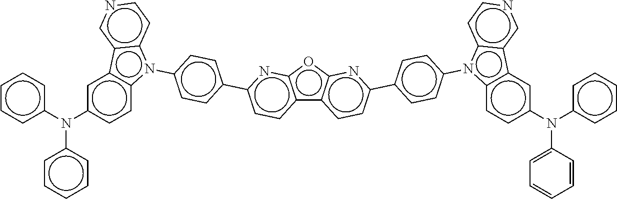 | M4 |
| 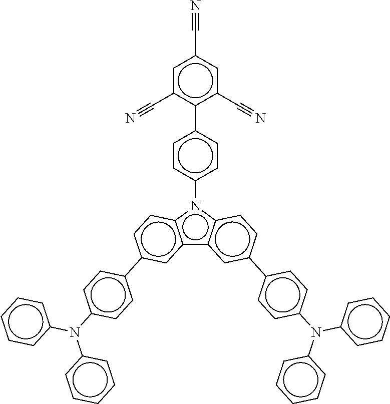 | M5 |
| 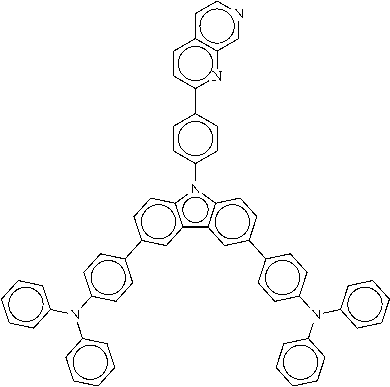 | M6 |
| 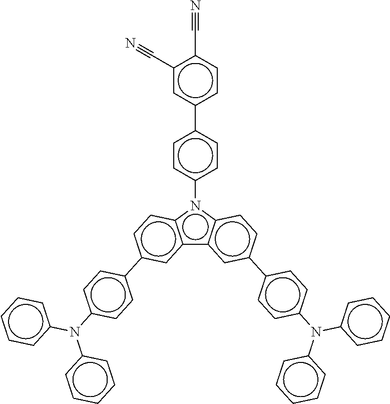 | M7 |
| 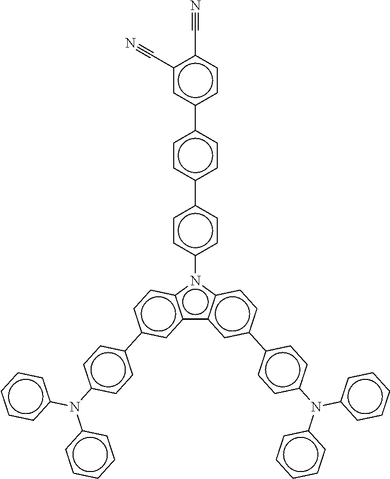 | M8 |
| 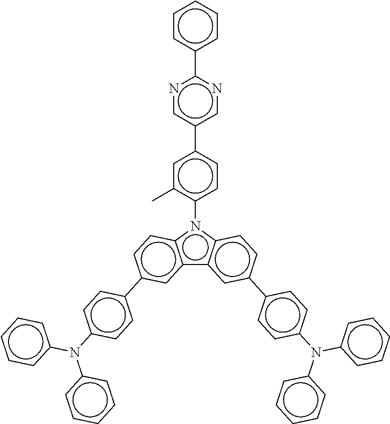 | M9 |
| 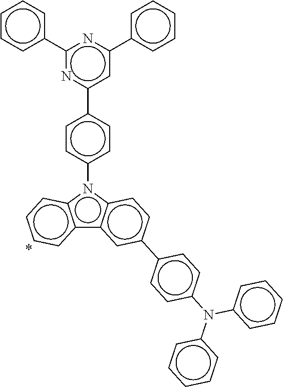 | M10 |
| 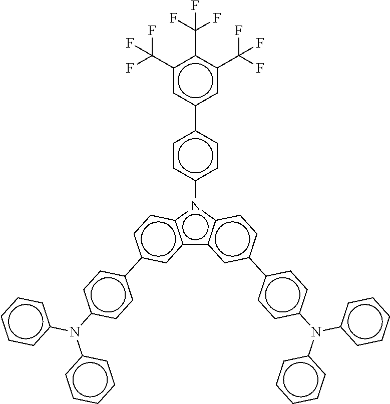 | M11 |
| 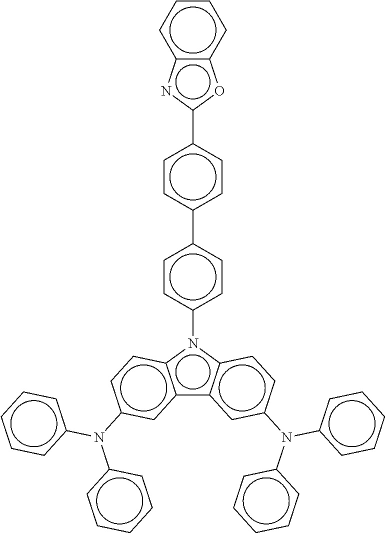 | M12 |
| 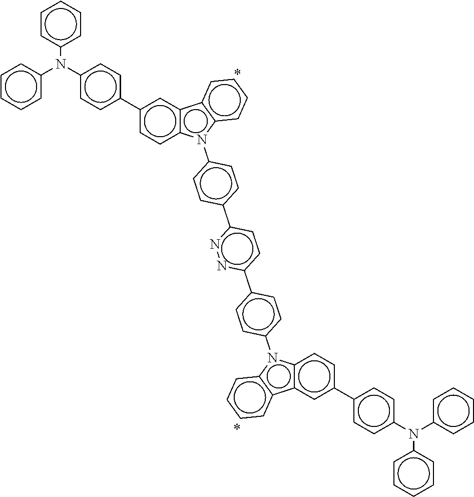 | M13 |
| 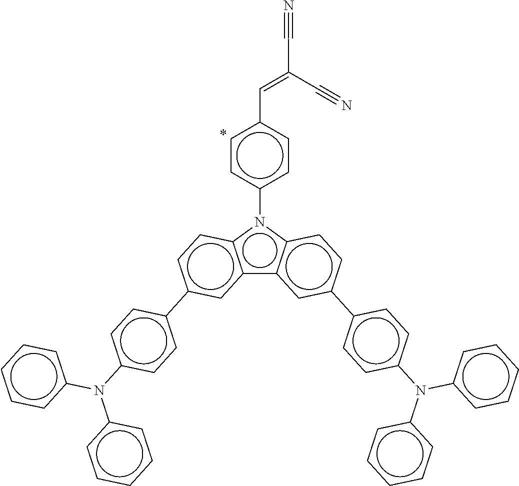 | M14 |
| 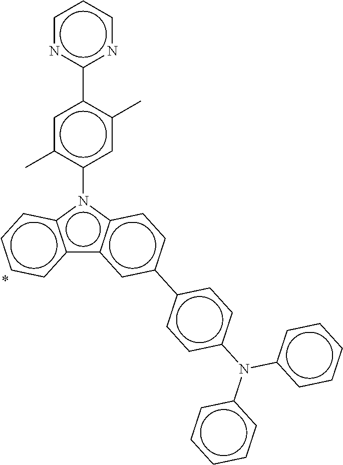 | M15 |
| 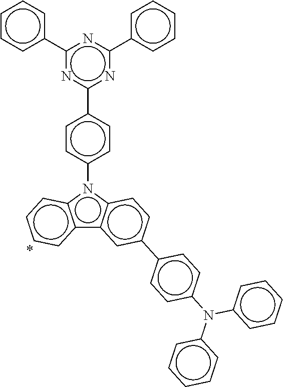 | M16 |
| 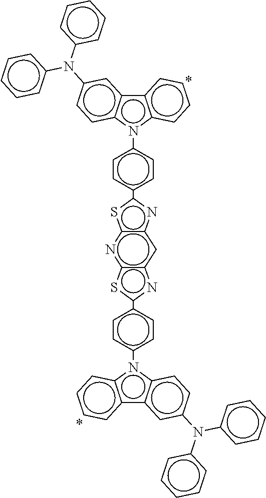 | M17 |
| 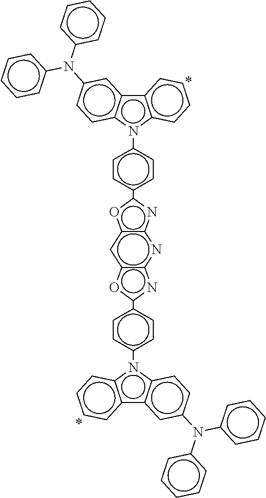 | M18 |
| 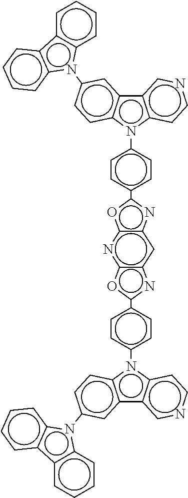 | M19 |
| 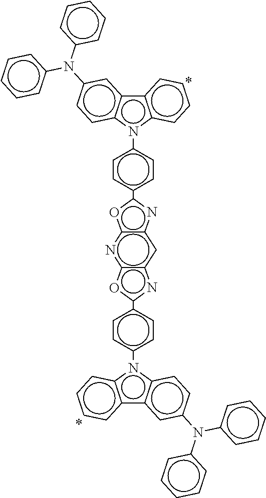 | M20 |
| 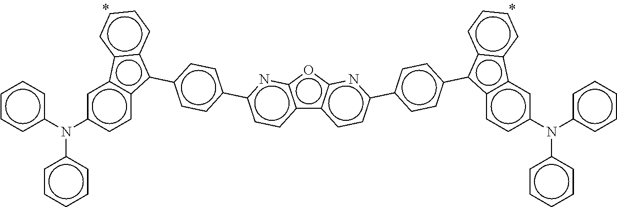 | M21 |
| 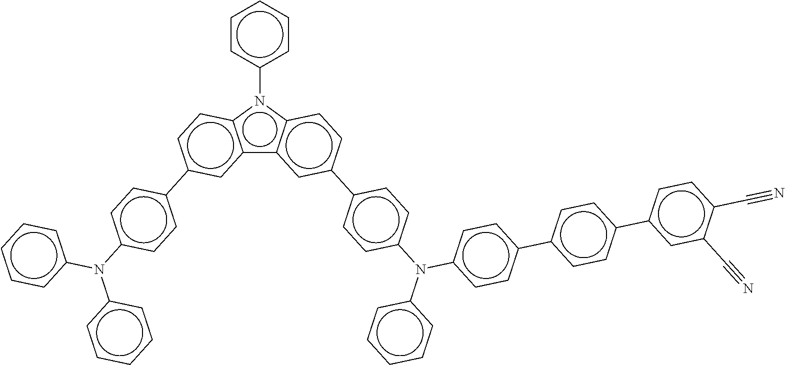 | M22 |
| 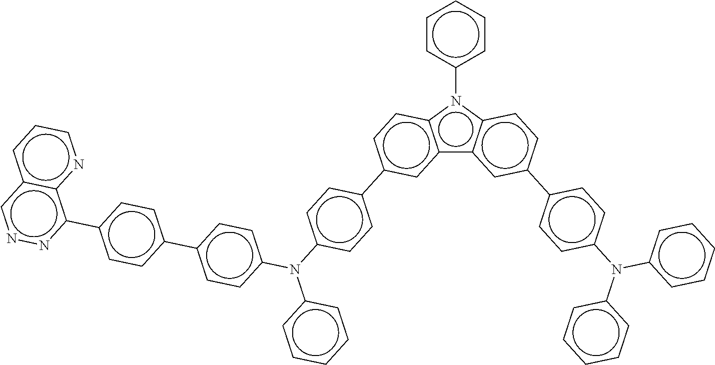 | M23 |
| 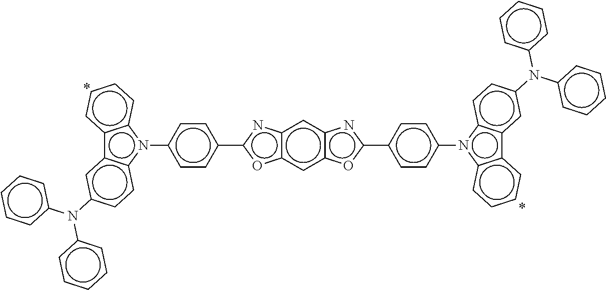 | M24 |
| 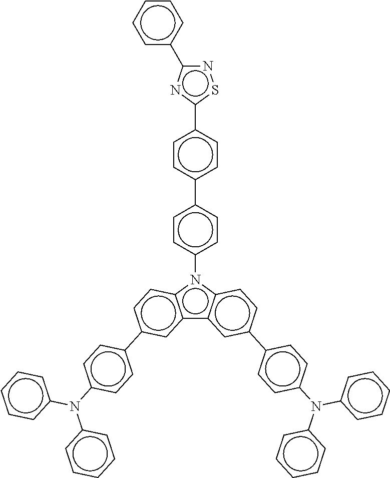 | M25 |
| 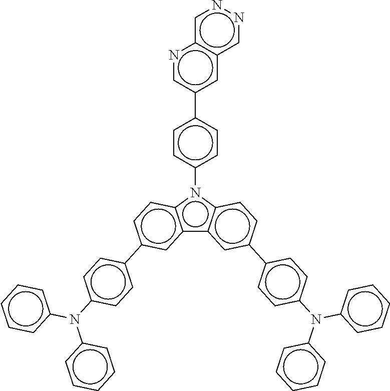 | M26 |
| 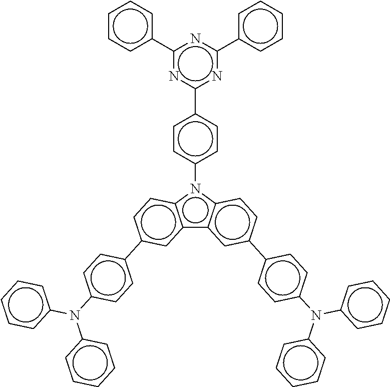 | M27 |
| 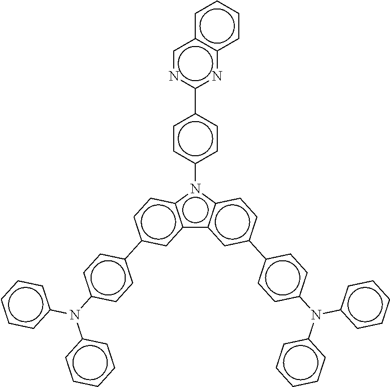 | M28 |
| 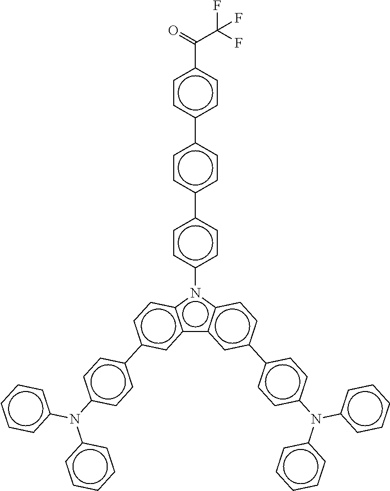 | M29 |
| 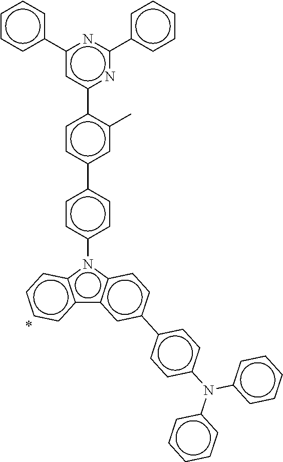 | M30 |
| 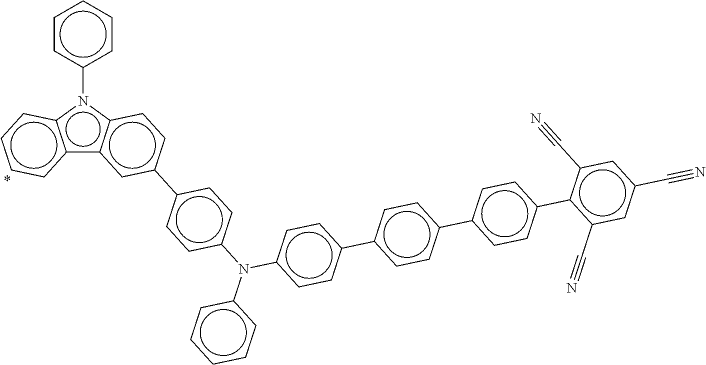 | M31 |
| 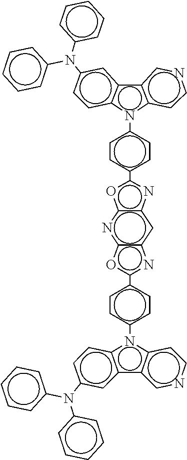 | M32 |
| 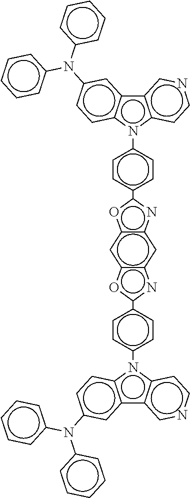 | M33 |
| 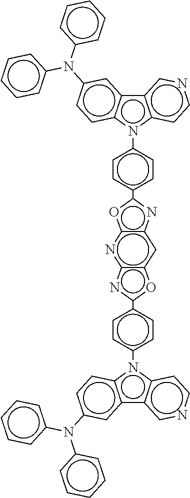 | M34 |
| 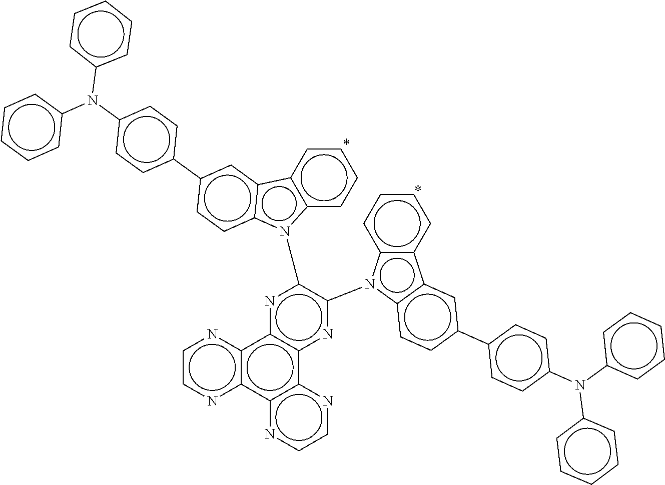 | M35 |
| 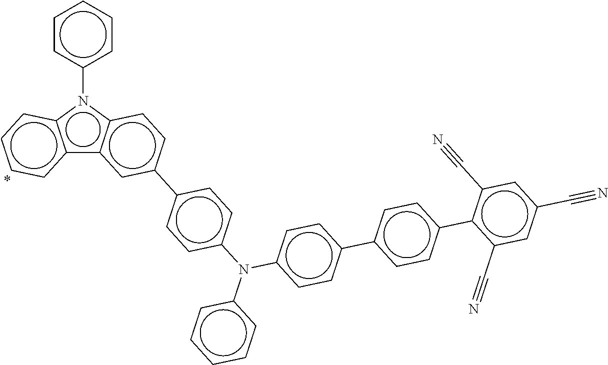 | M36 |
| 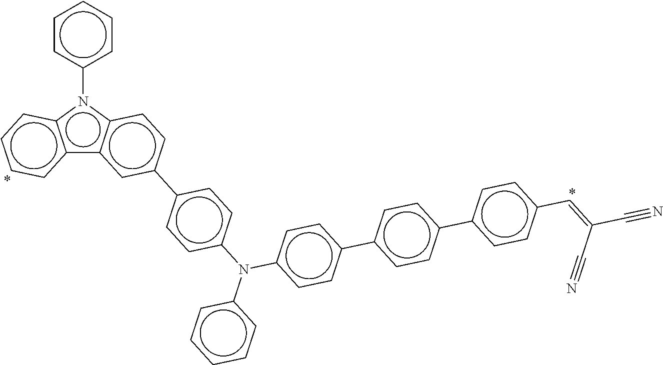 | M37 |
| 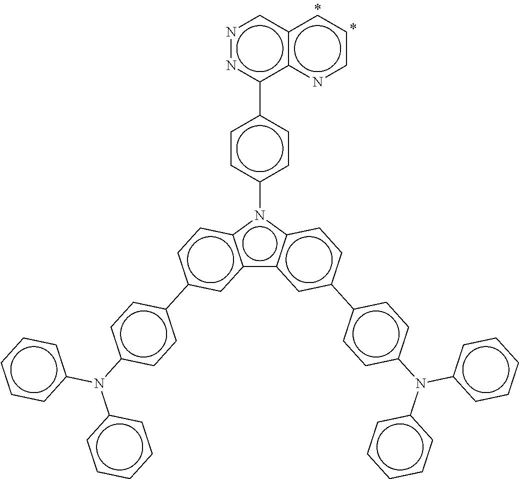 | M38 |
| 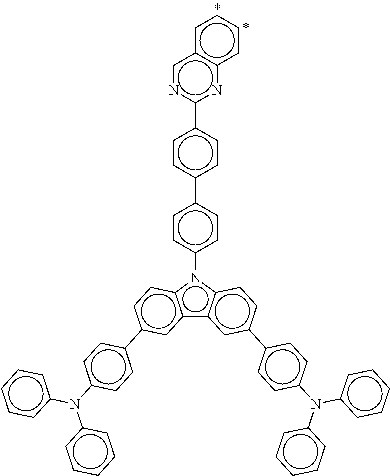 | M39 |
| 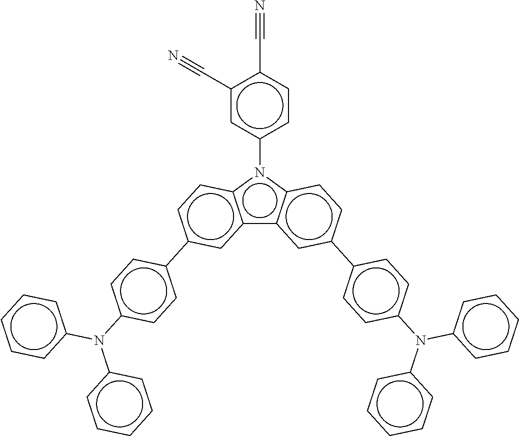 | M40 |
| 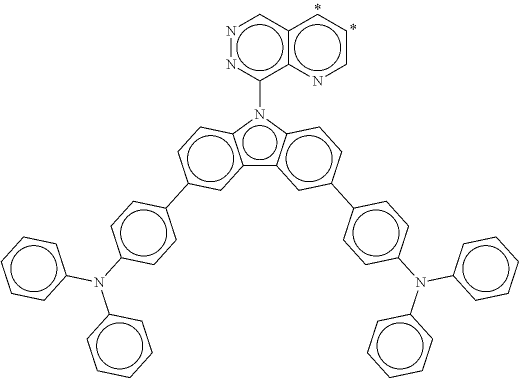 | M41 |
| 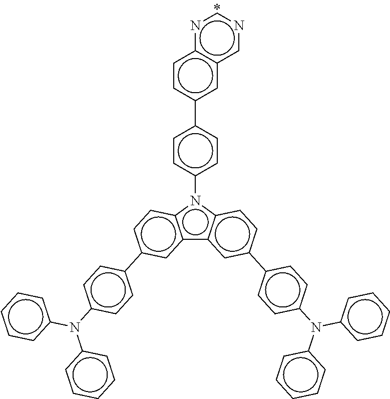 | M42 |
| 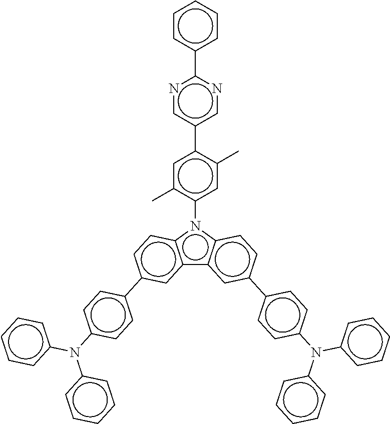 | M43 |
| 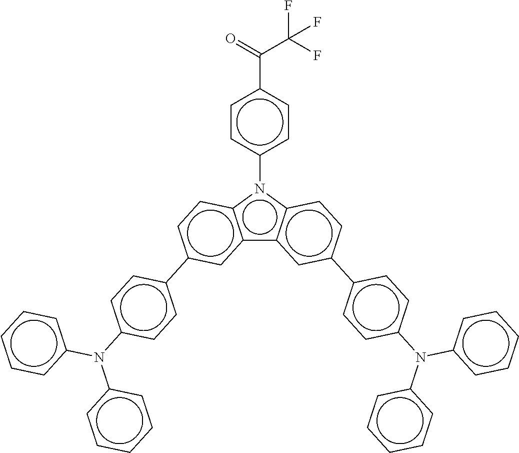 | M44 |
| 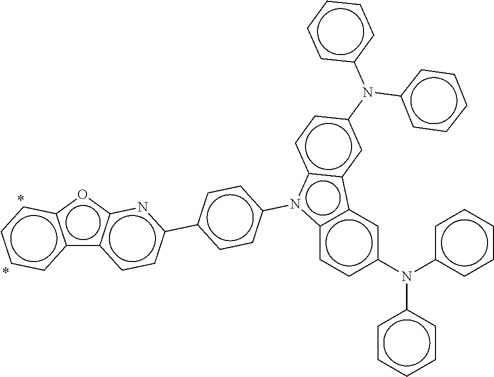 | M45 |
| 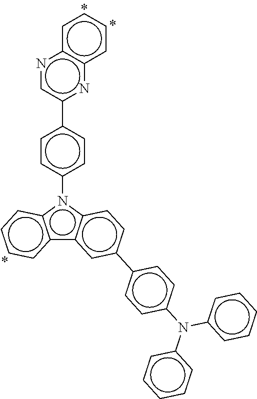 | M46 |
| 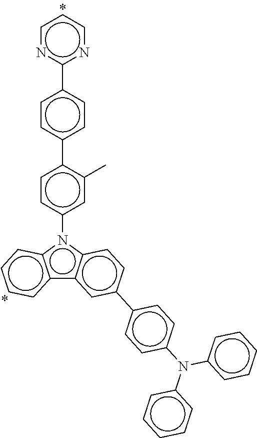 | M47 |
| 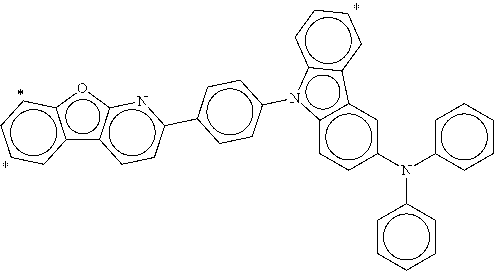 | M48 |
| 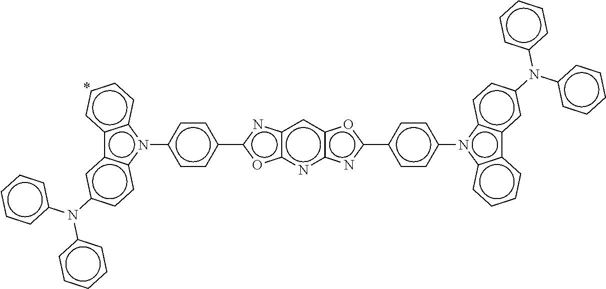 | M49 |
| 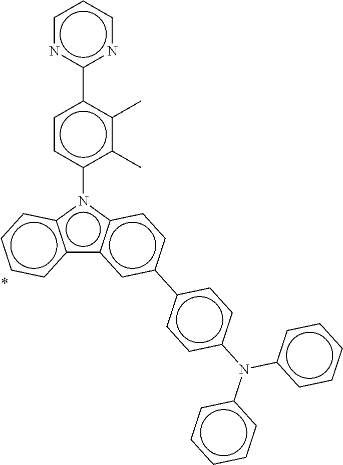 | M50 |
| 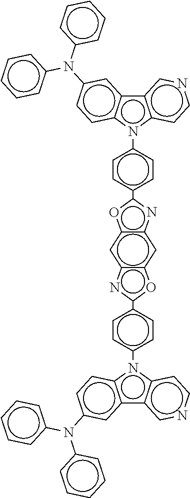 | M51 |
| 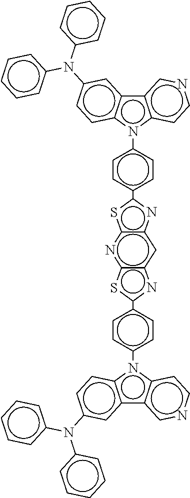 | M52 |
| 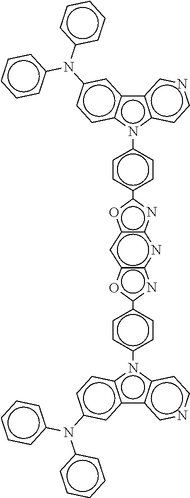 | M53 |

In an example embodiment of the fourteenth aspect, the present invention is any one molecule selected from compounds N1 to N151, listed in Table N. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE N

| | |
|---|---|
| 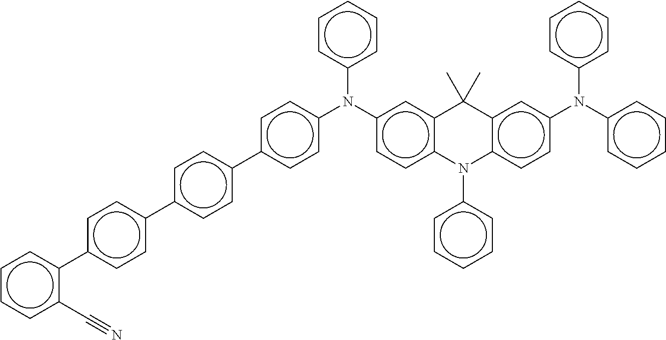 | N1 |
| 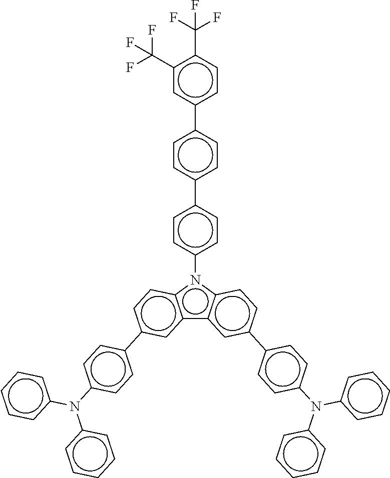 | N2 |
| 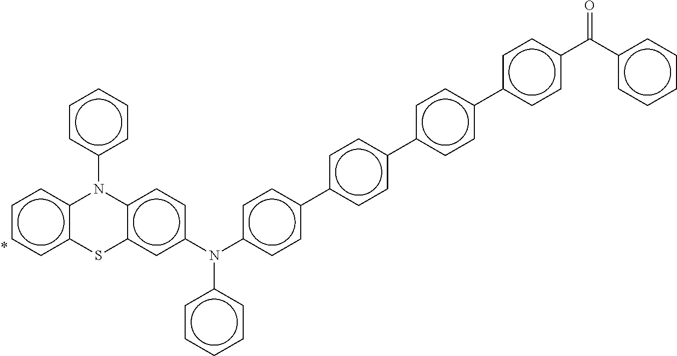 | N3 |
| 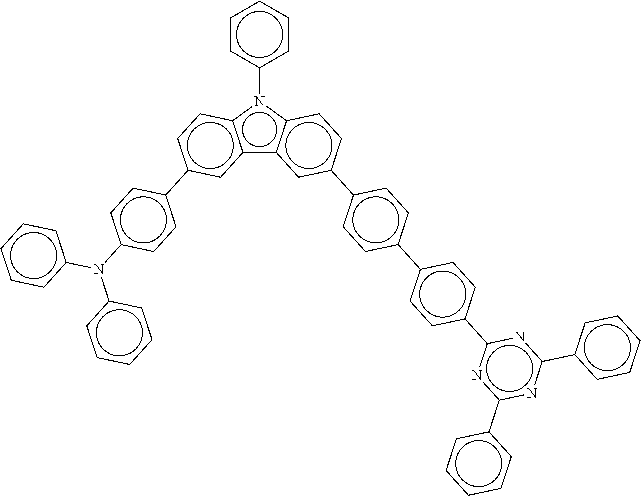 | N4 |
| 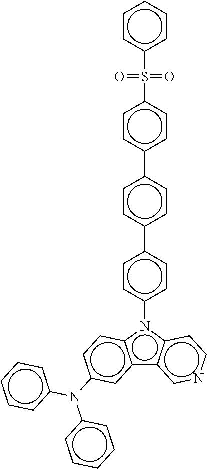 | N5 |
| 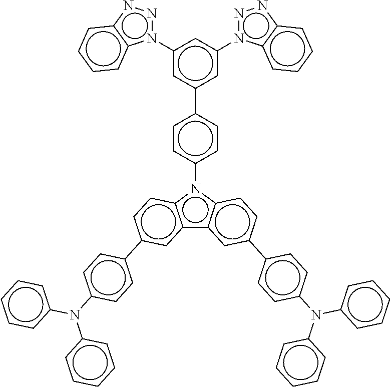 | N6 |
| 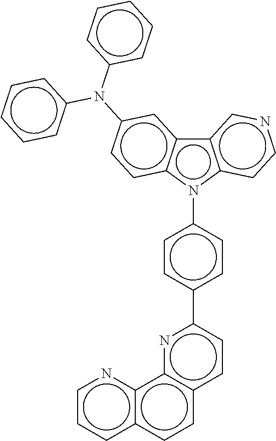 | N7 |
| 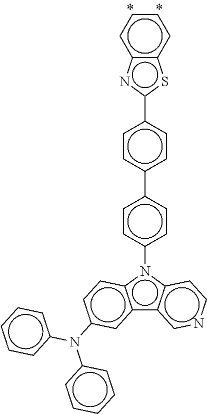 | N8 |
| 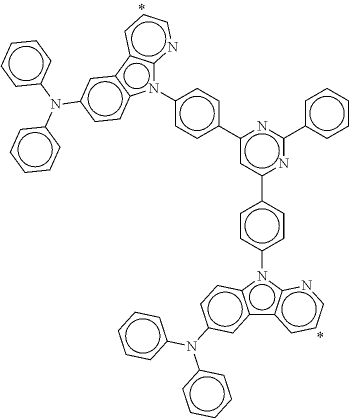 | N9 |
| 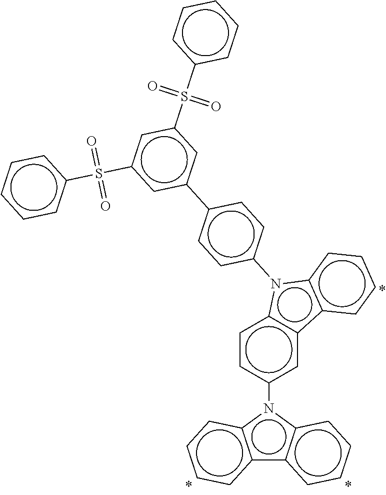 | N10 |
| 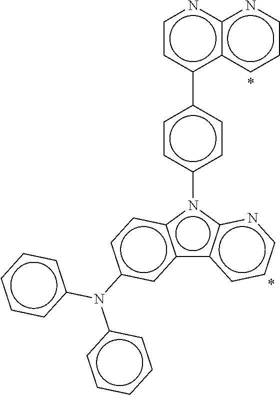 | N11 |
| 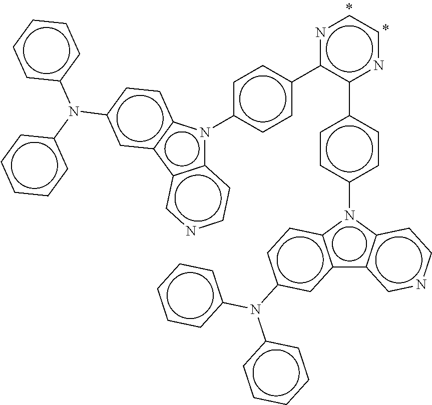 | N12 |
| 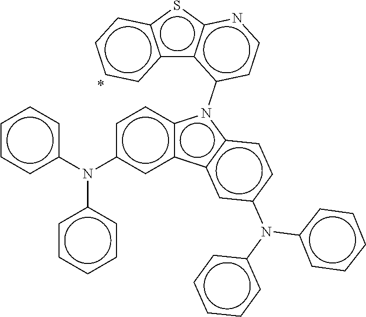 | N13 |
| 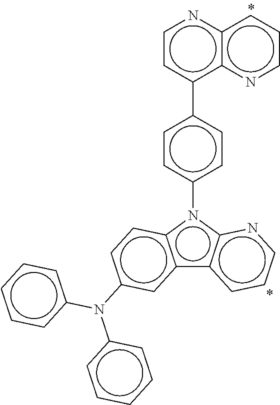 | N14 |
| 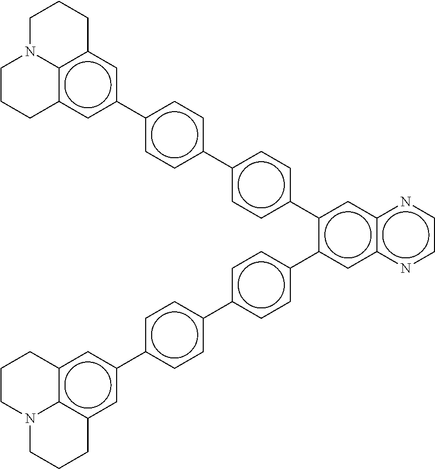 | N15 |
| 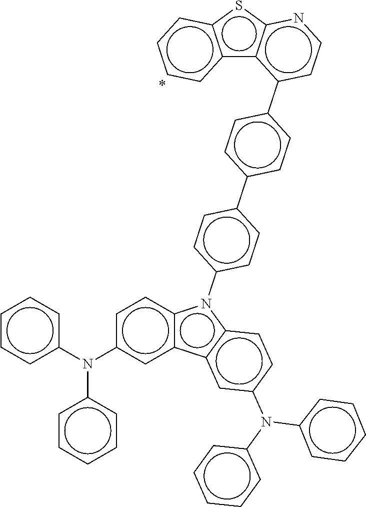 | N16 |
| 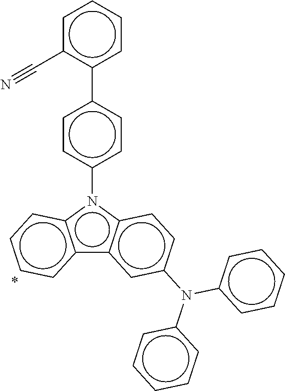 | N17 |
| 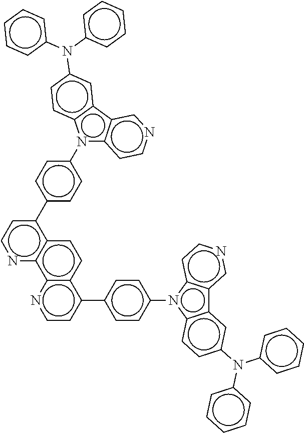 | N18 |
| 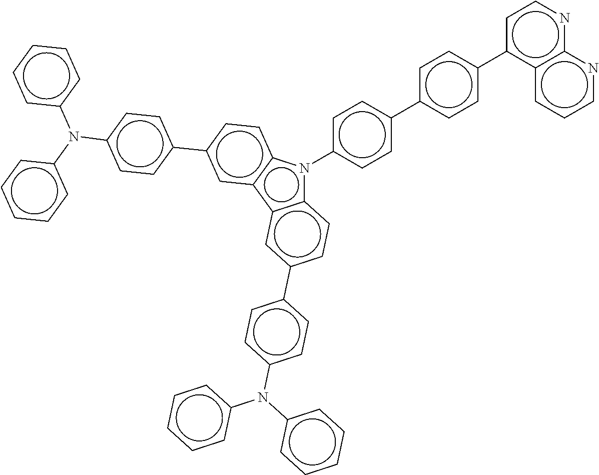 | N19 |
| 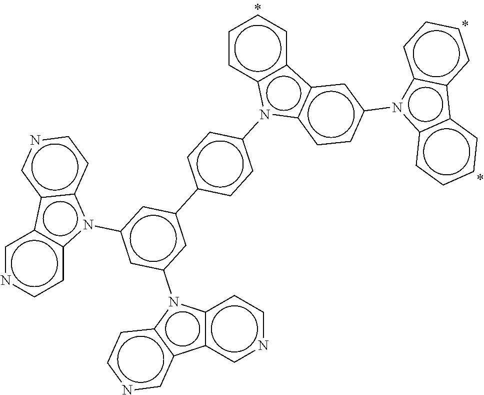 | N20 |
| 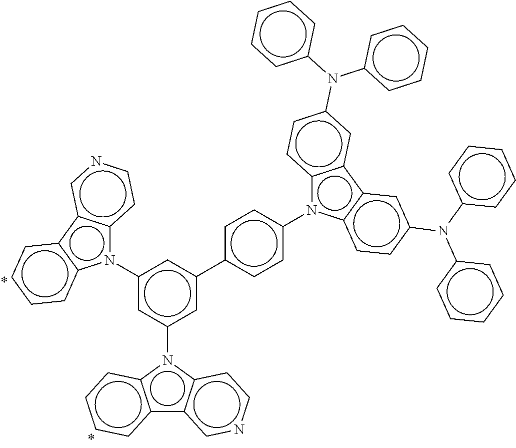 | N21 |
| 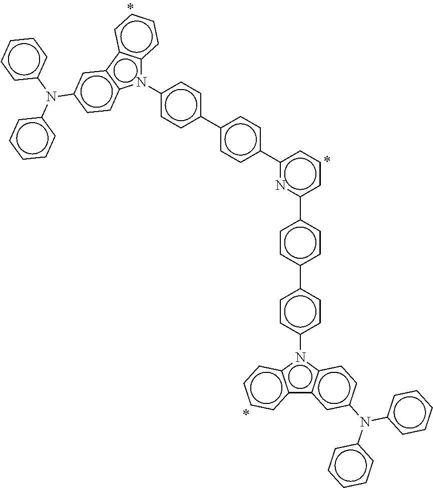 | N22 |
| 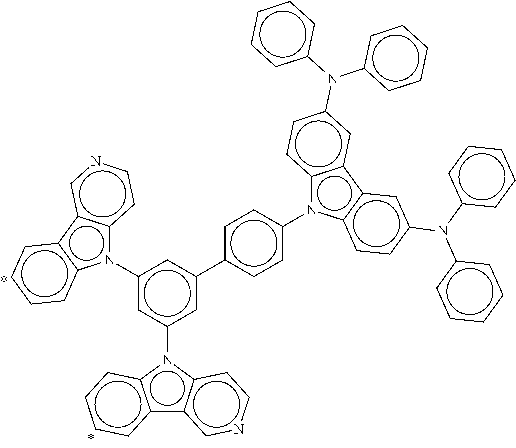 | N23 |
| 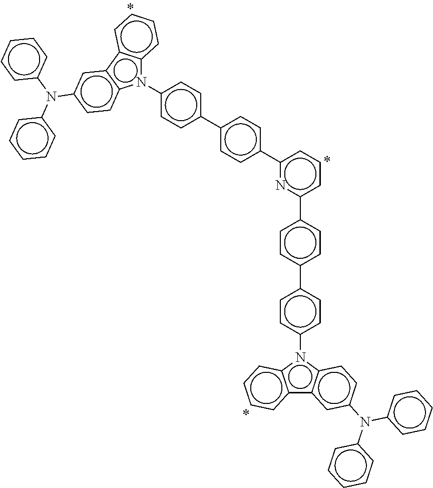 | N24 |
| 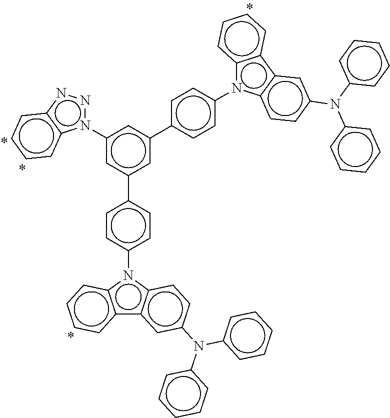 | N25 |
| 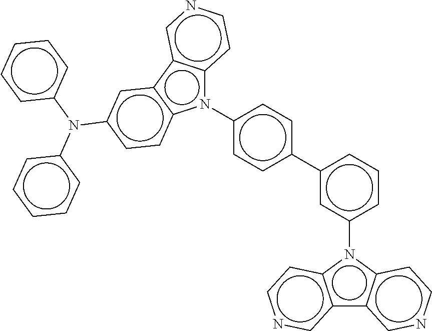 | N26 |
| 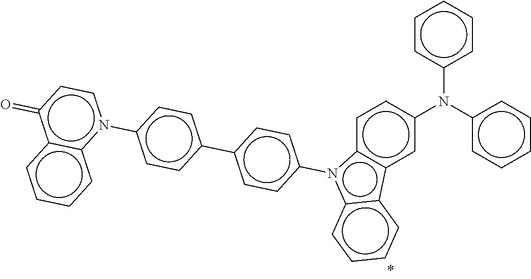 | N27 |
| 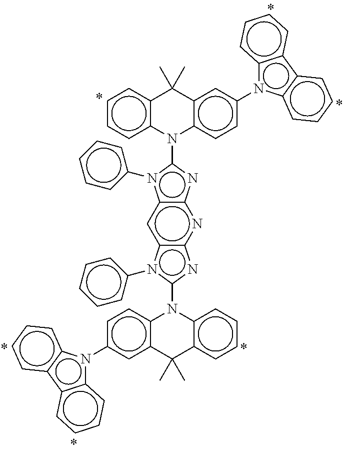 | N28 |
| 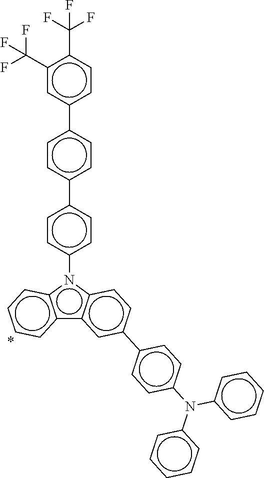 | N29 |
| 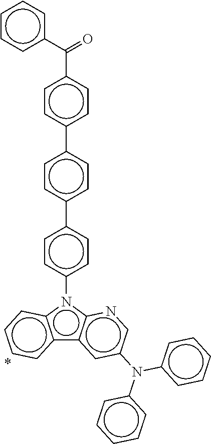 | N30 |
| 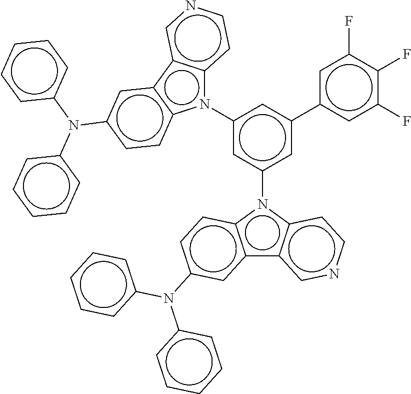 | N31 |
| 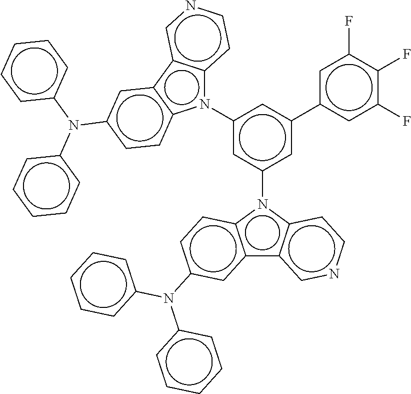 | N32 |
| 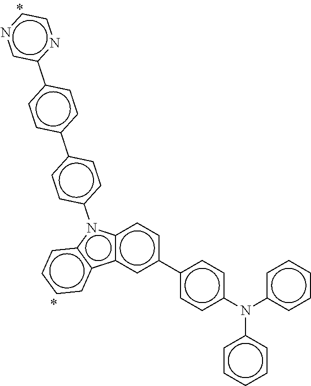 | N33 |
| 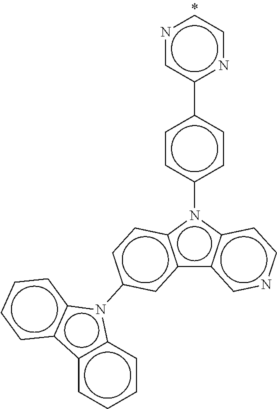 | N34 |
| 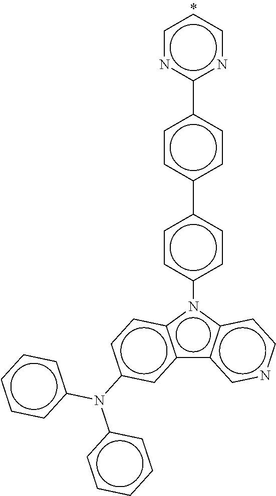 | N35 |
| 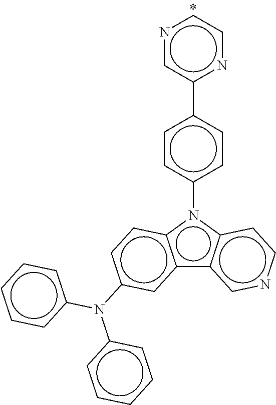 | N36 |
| 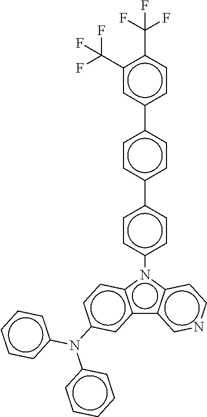 | N37 |
| 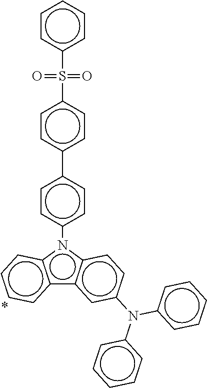 | N38 |
| 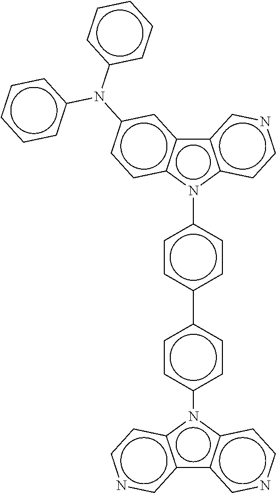 | N39 |
| 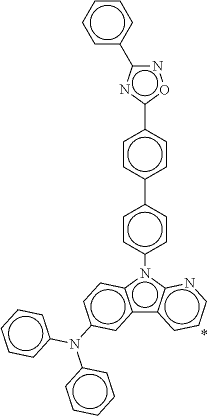 | N40 |
| 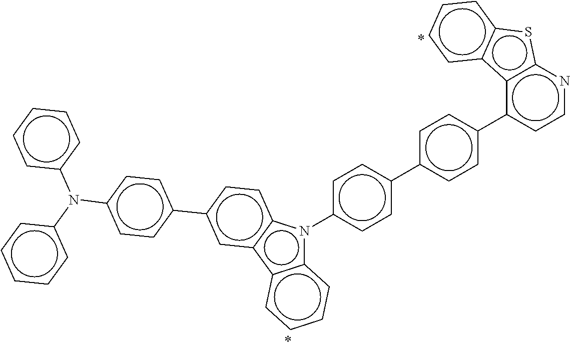 | N41 |
| 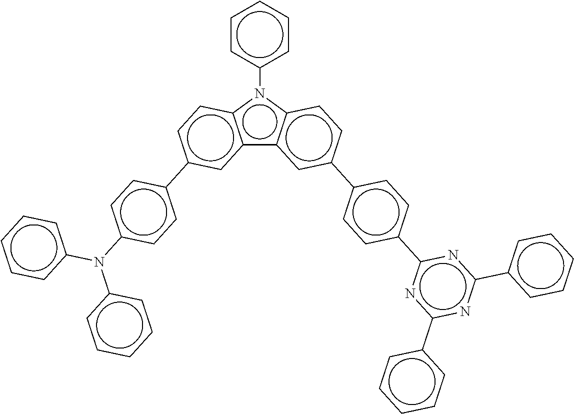 | N42 |
| 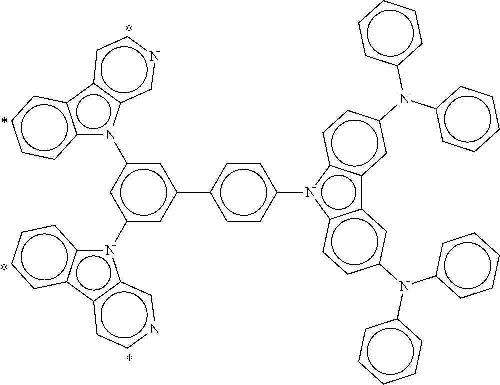 | N43 |
| 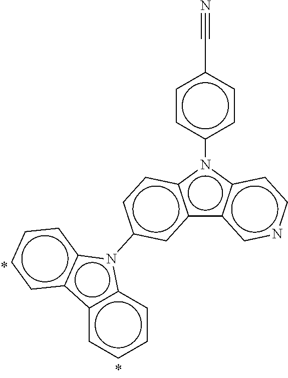 | N44 |
| 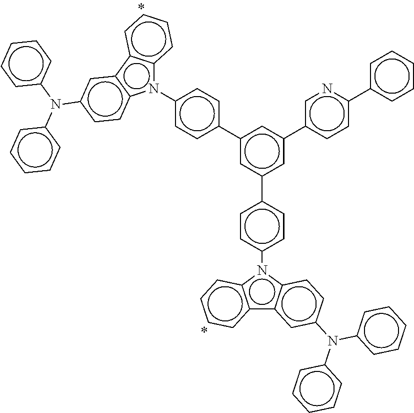 | N45 |
| 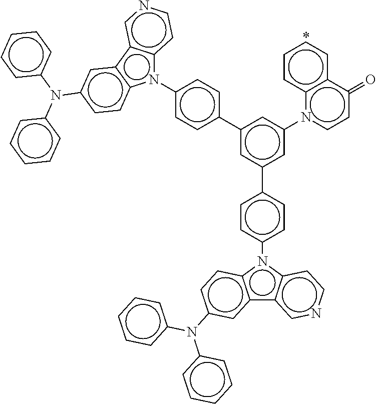 | N46 |
| 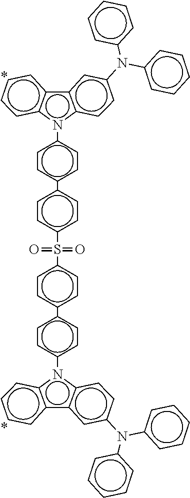 | N47 |
| 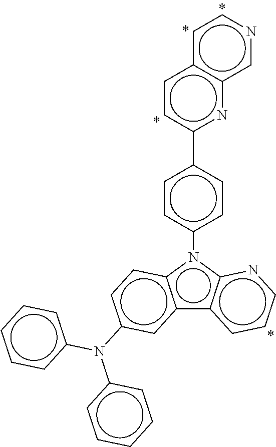 | N48 |
| 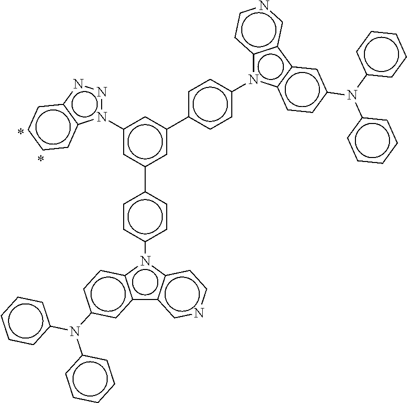 | N49 |
| 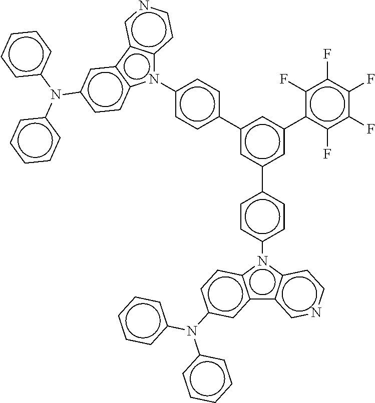 | N50 |
| 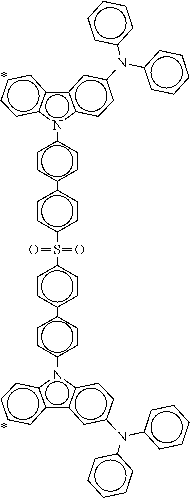 | N51 |
| 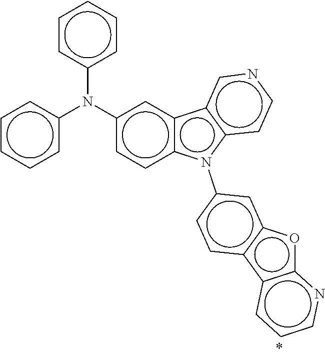 | N52 |
| 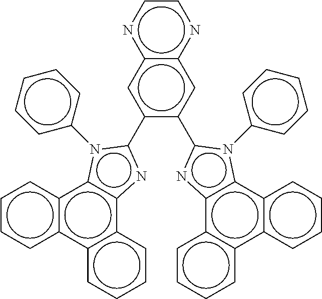 | N53 |
| 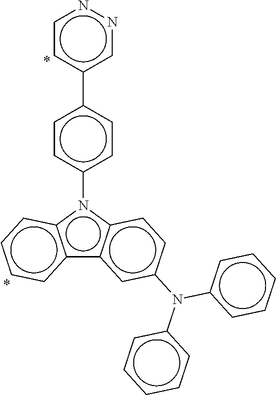 | N54 |
| 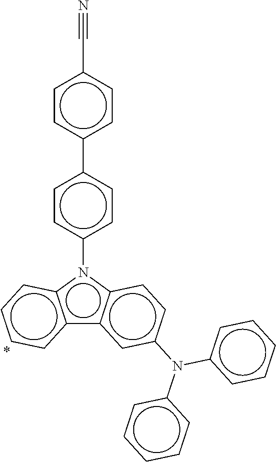 | N55 |
| 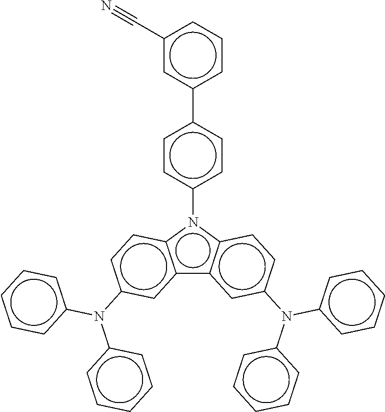 | N56 |
| 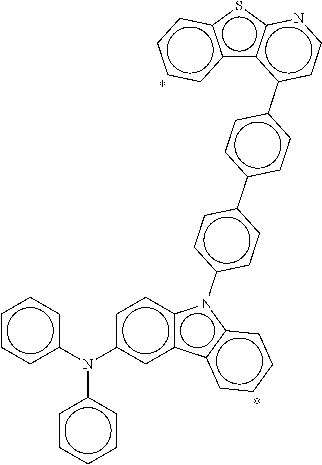 | N57 |
| 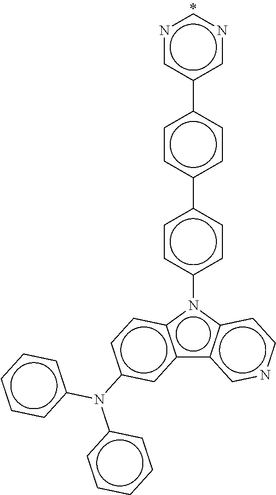 | N58 |
| 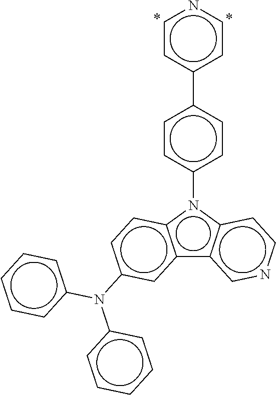 | N59 |
| 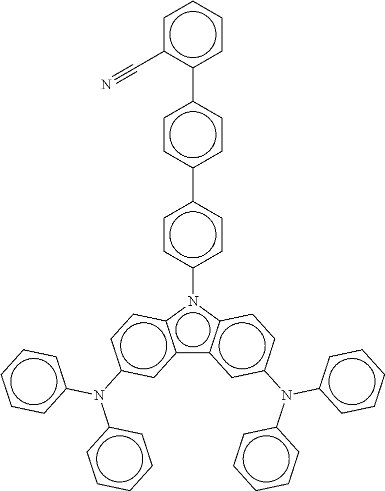 | N60 |
| 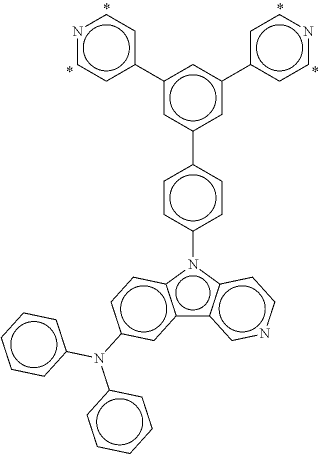 | N61 |
| 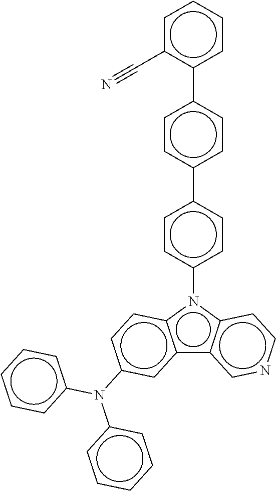 | N62 |
| 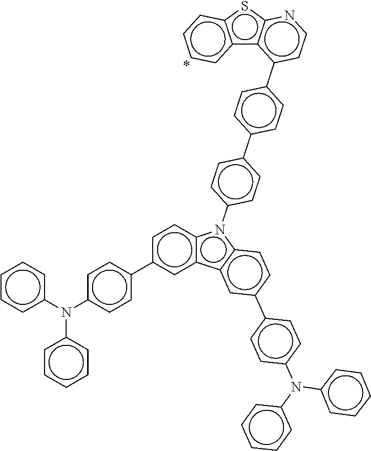 | N63 |
| 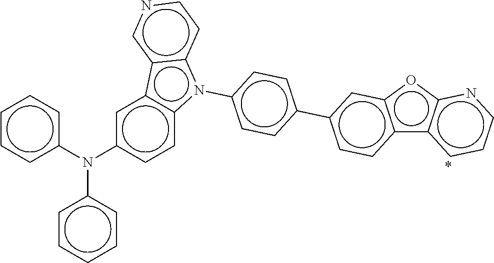 | N64 |
| 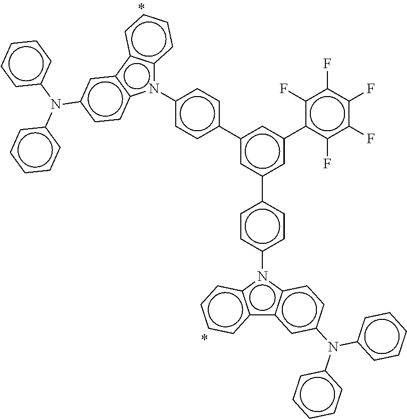 | N65 |
| 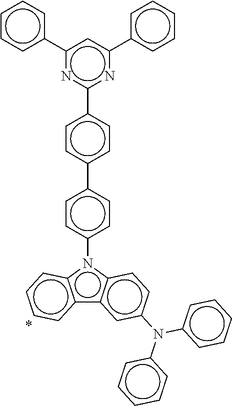 | N66 |
| 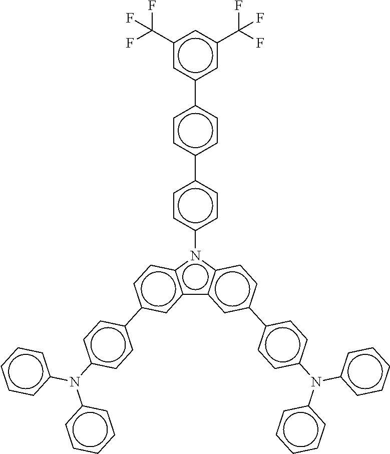 | N67 |
| 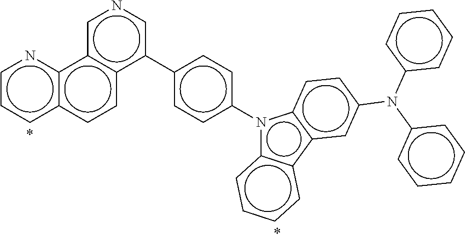 | N68 |
| 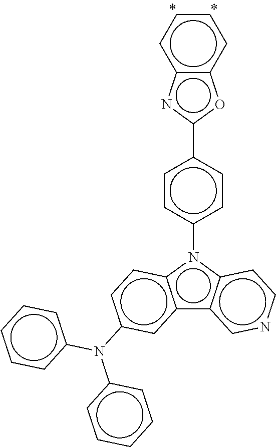 | N69 |
| 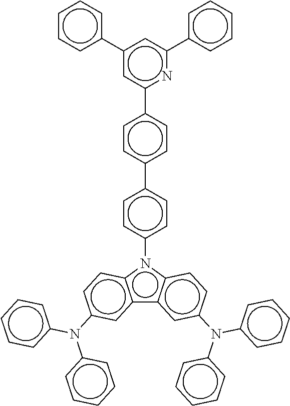 | N70 |
| 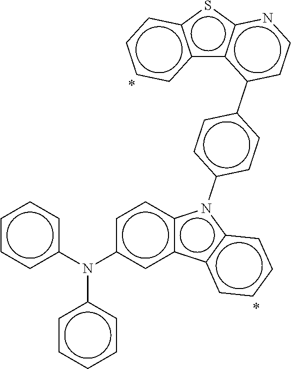 | N71 |
| 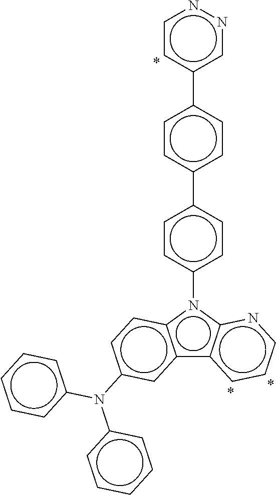 | N72 |
| 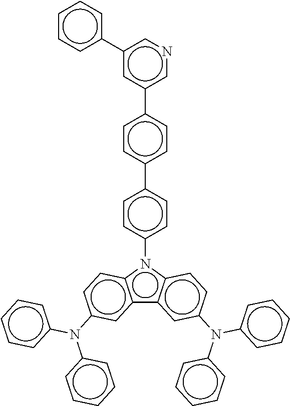 | N73 |
| 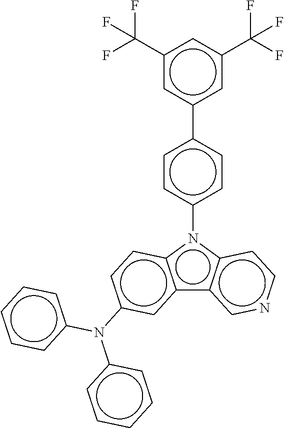 | N74 |
| 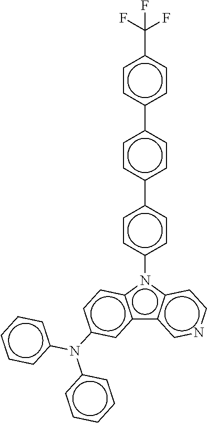 | N75 |
| 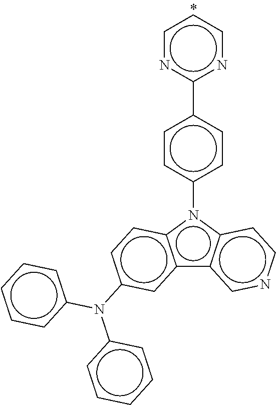 | N76 |
| 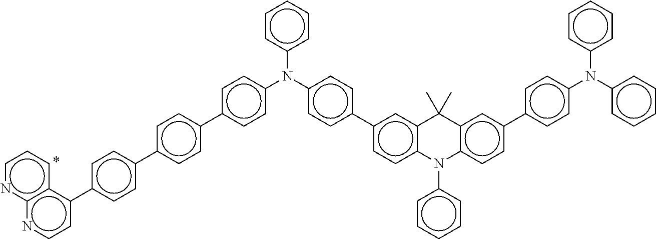 | N77 |
| 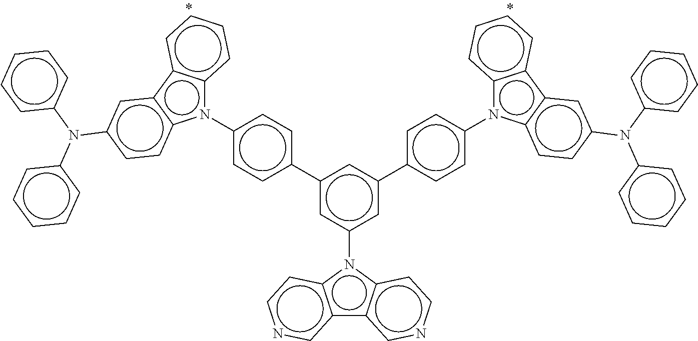 | N78 |
| 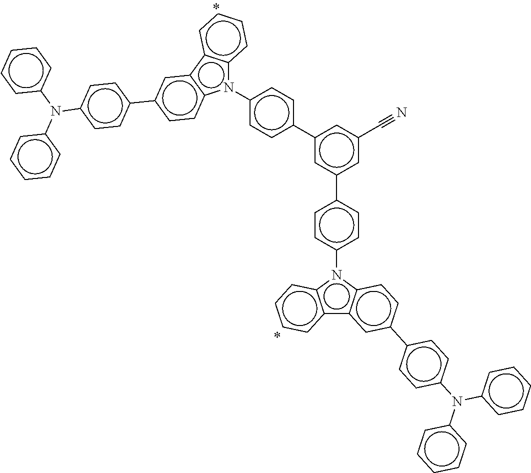 | N79 |
| 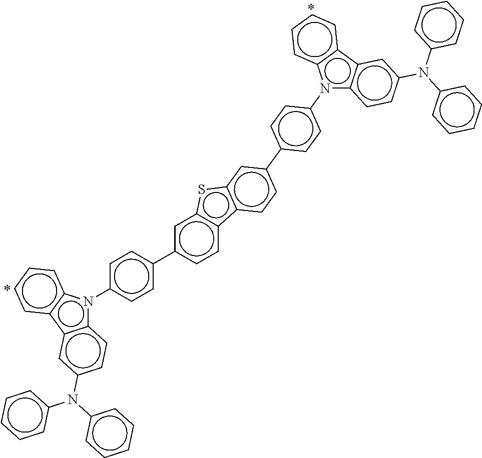 | N80 |
| 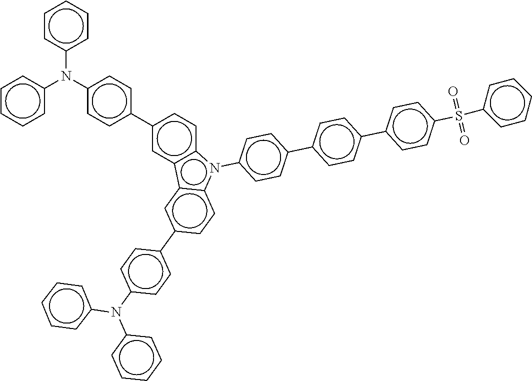 | N81 |
| 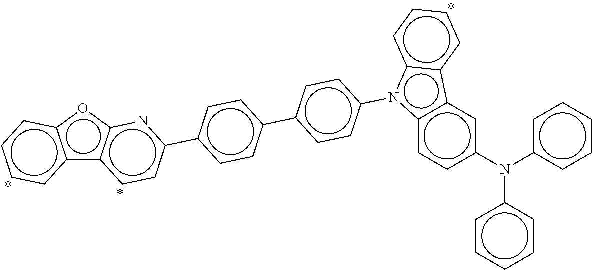 | N82 |
| 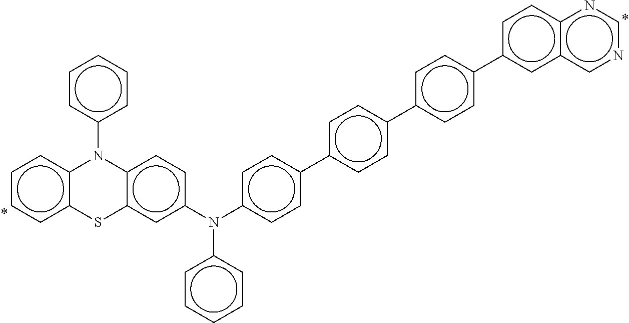 | N83 |
| 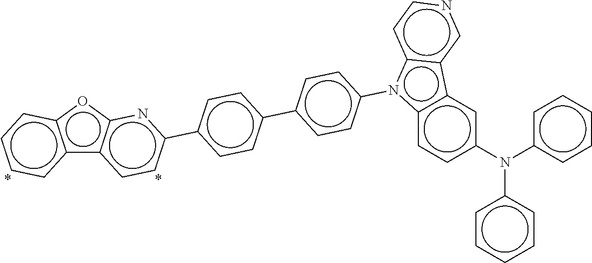 | N84 |
| 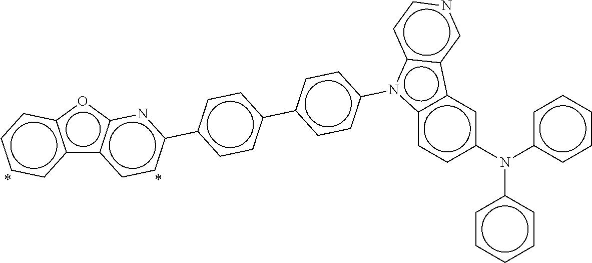 | N85 |
| 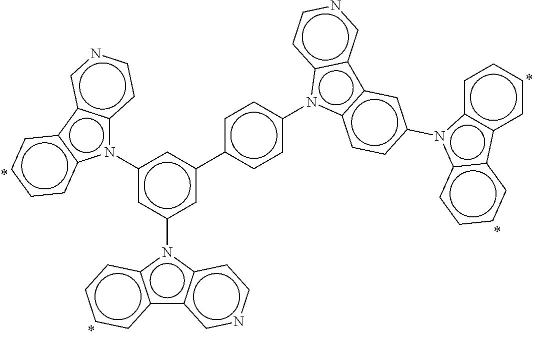 | N86 |
| 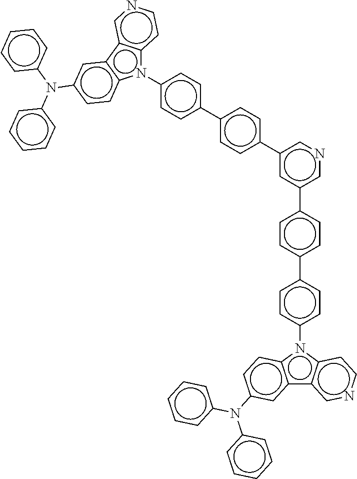 | N87 |
| 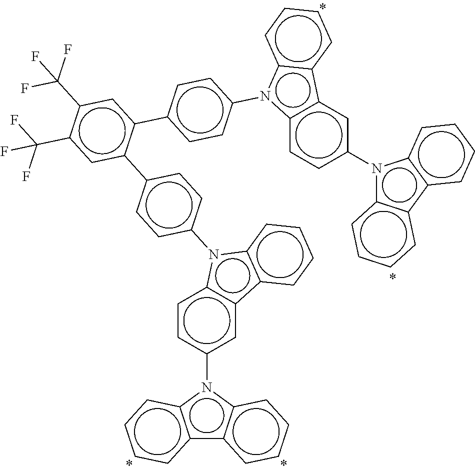 | N88 |
| 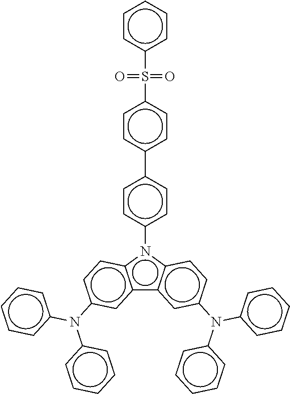 | N89 |
| 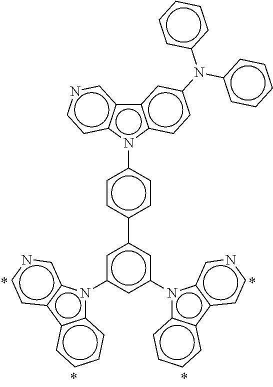 | N90 |
| 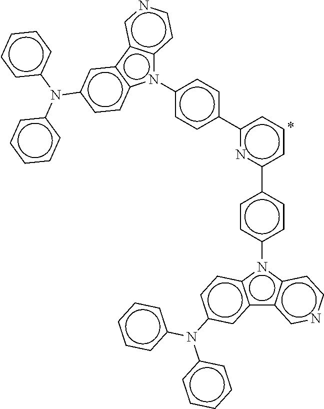 | N91 |
| 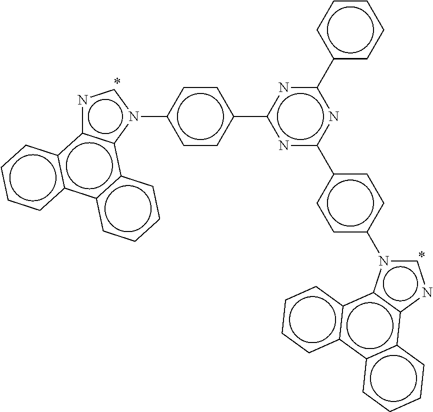 | N92 |
| 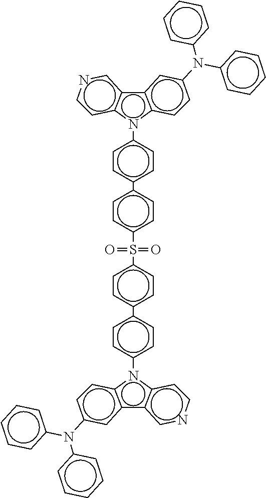 | N93 |
| 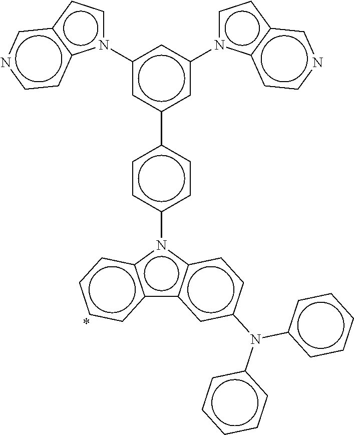 | N94 |
| 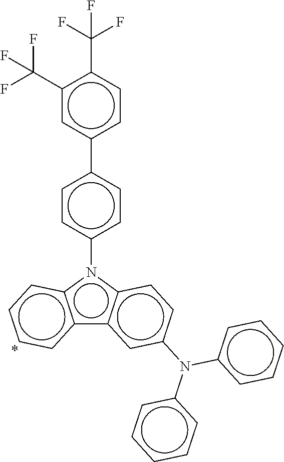 | N95 |
| 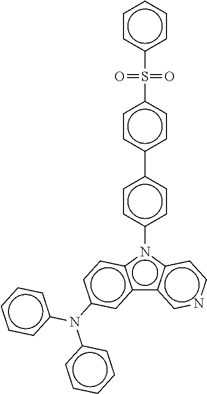 | N96 |
| 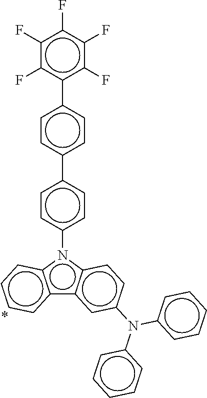 | N97 |
| 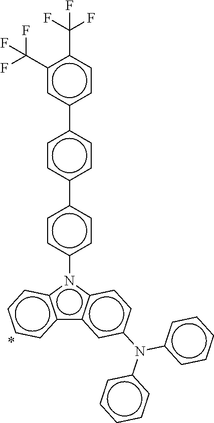 | N98 |
| 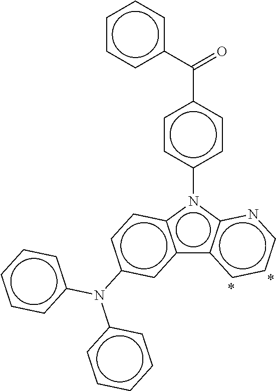 | N99 |
| 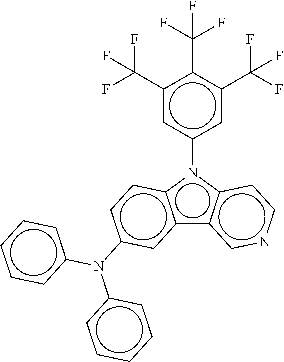 | N100 |
| 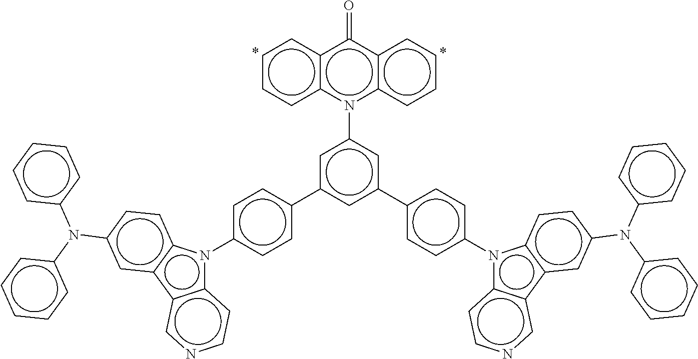 | N101 |
| 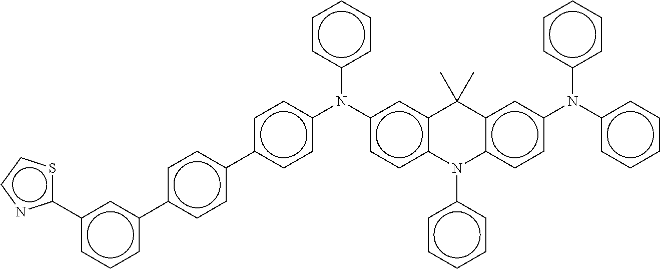 | N102 |
| 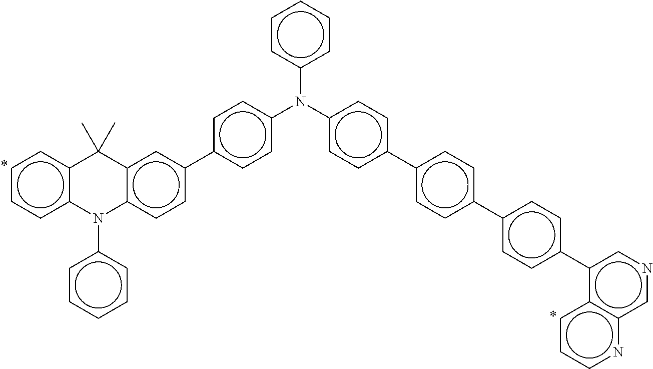 | N103 |
| 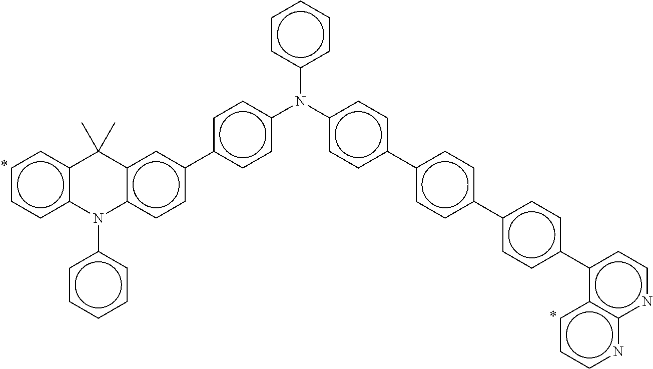 | N104 |
| 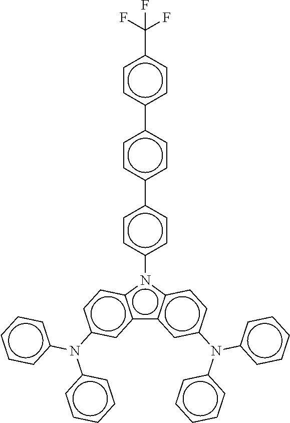 | N105 |
| 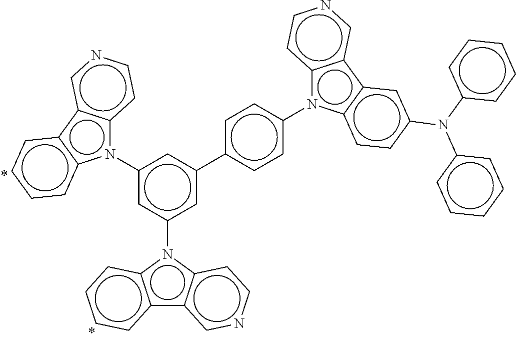 | N106 |
| 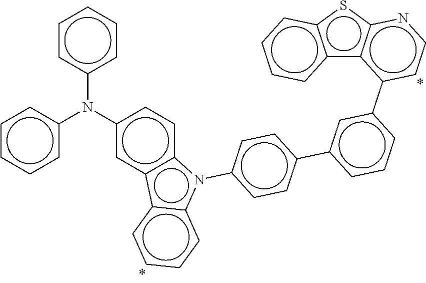 | N107 |
| 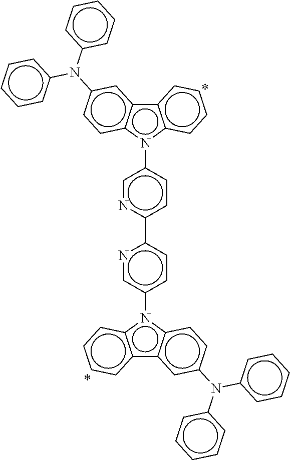 | N108 |
| 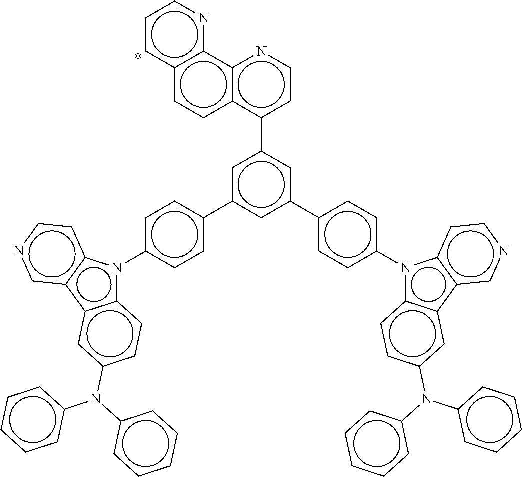 | N109 |
| 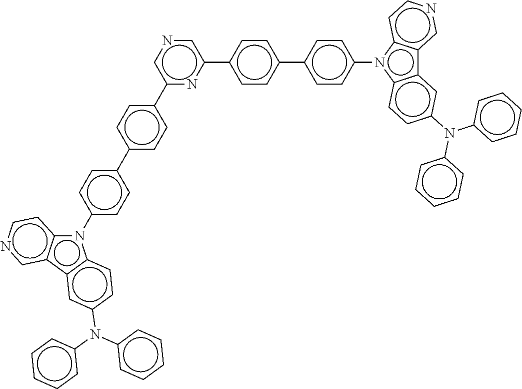 | N110 |
| 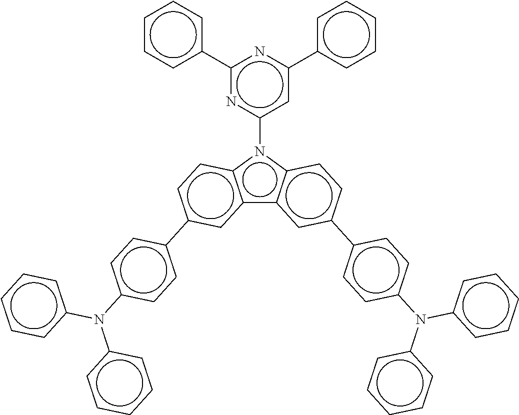 | N111 |
| 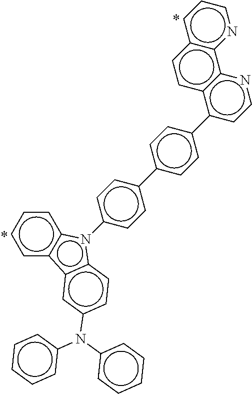 | N112 |
| 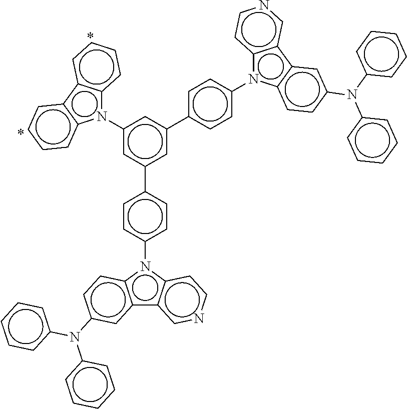 | N113 |
| 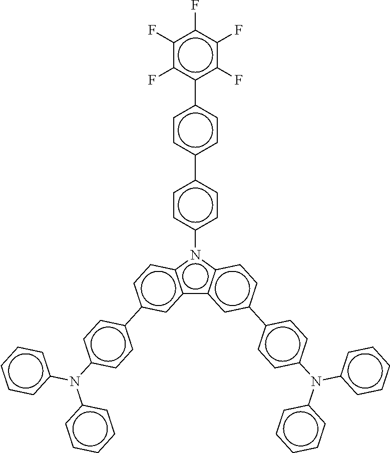 | N114 |
| 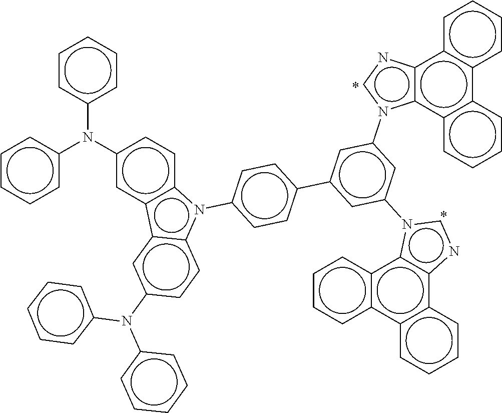 | N115 |
| 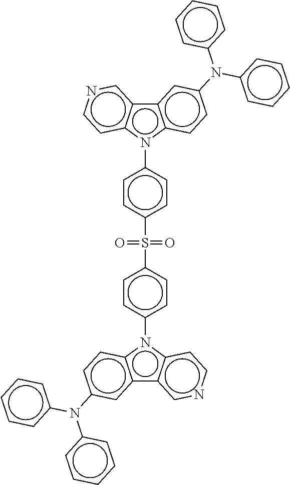 | N116 |
| 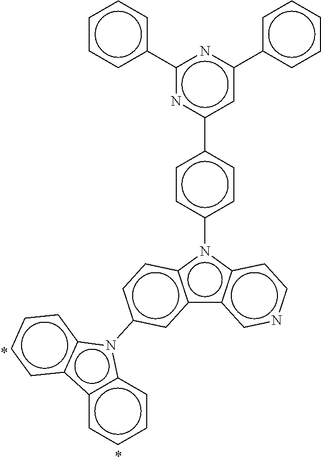 | N117 |
| 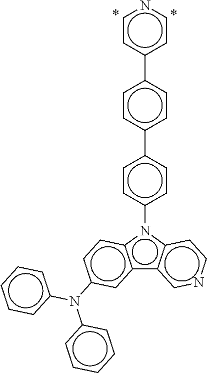 | N118 |
| 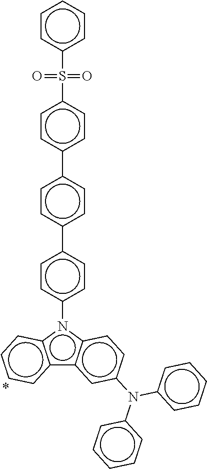 | N119 |
| 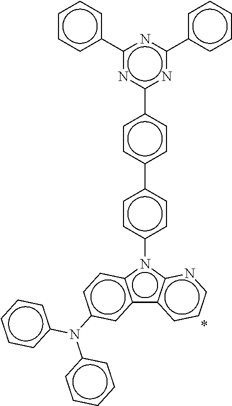 | N120 |
| 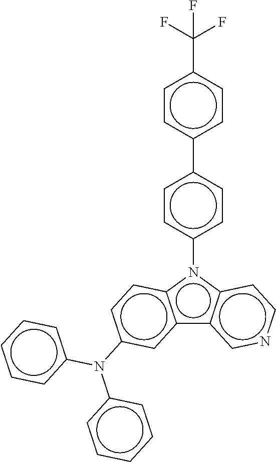 | N121 |
| 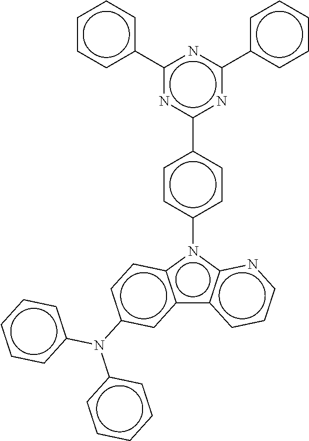 | N122 |
| 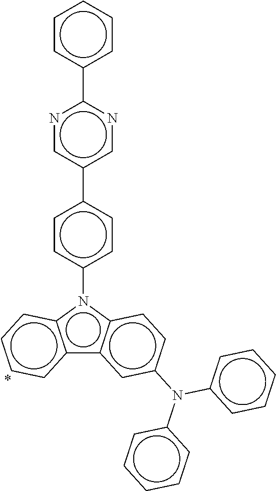 | N123 |
| 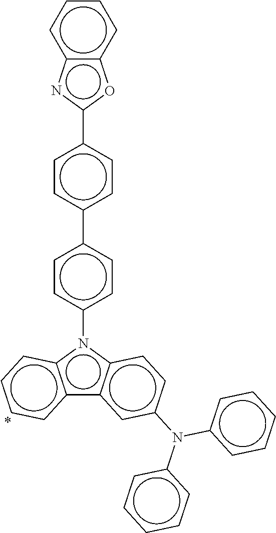 | N124 |
| 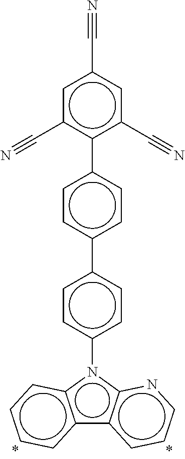 | N125 |
| 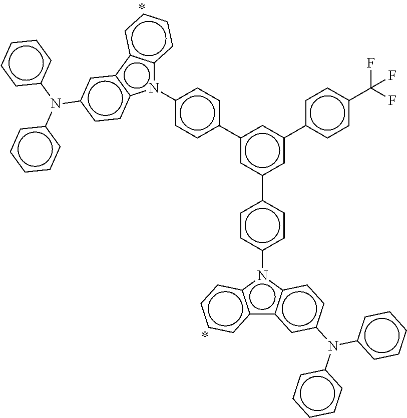 | N126 |
| 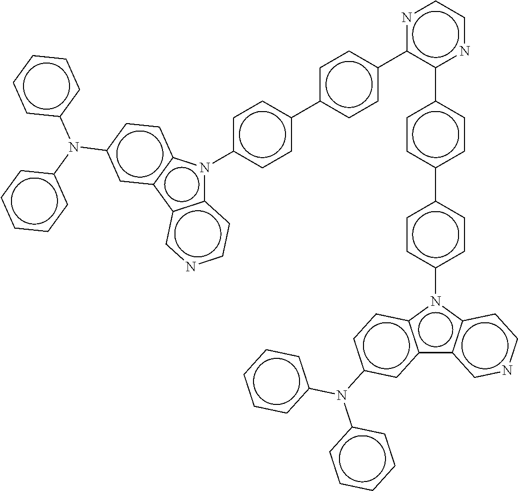 | N127 |
| 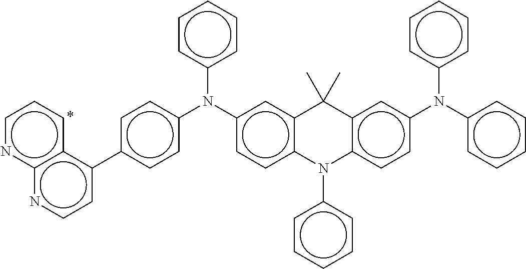 | N128 |
| 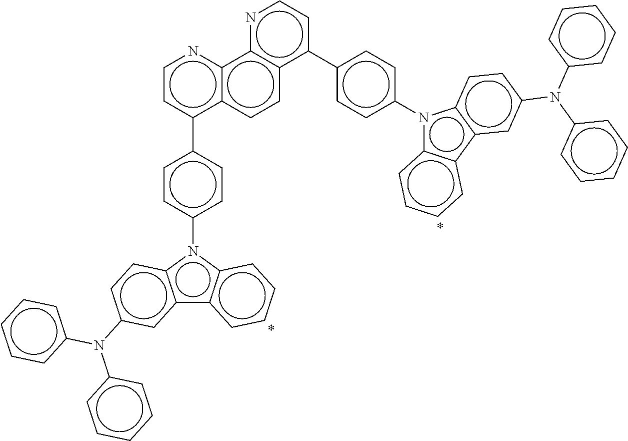 | N129 |
| 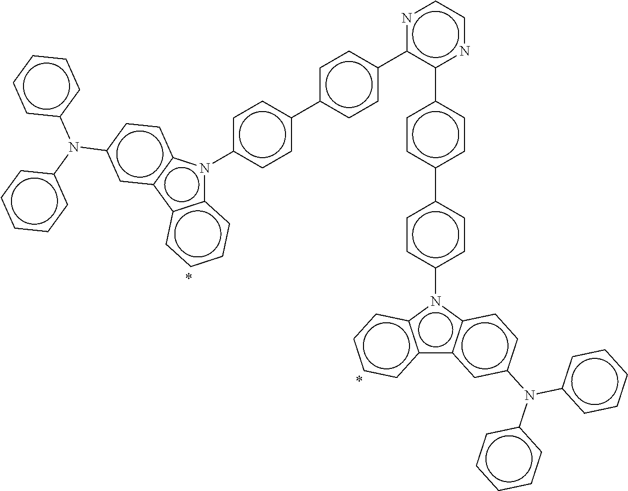 | N130 |
| 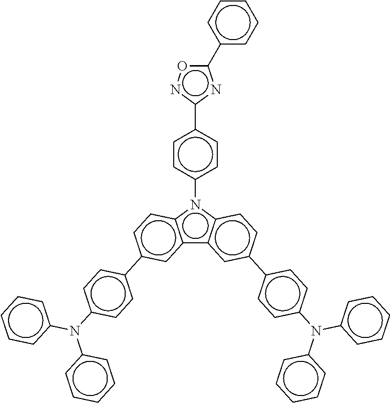 | N131 |
| 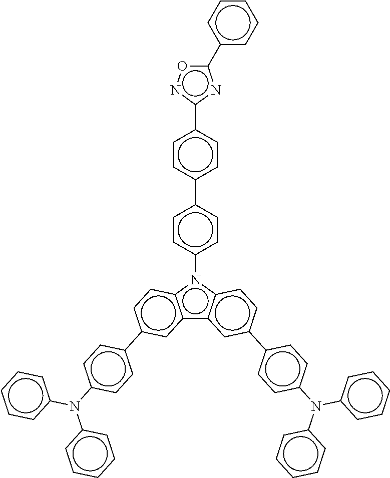 | N132 |
| 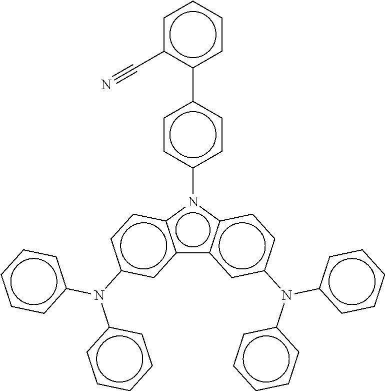 | N133 |
| 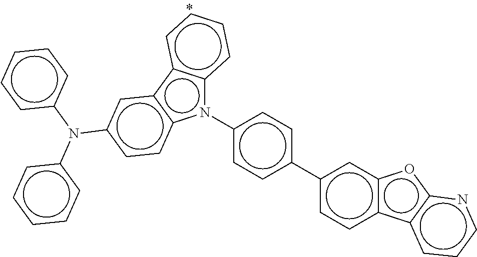 | N134 |
| 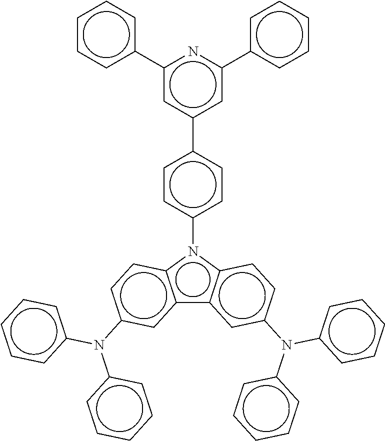 | N135 |
| 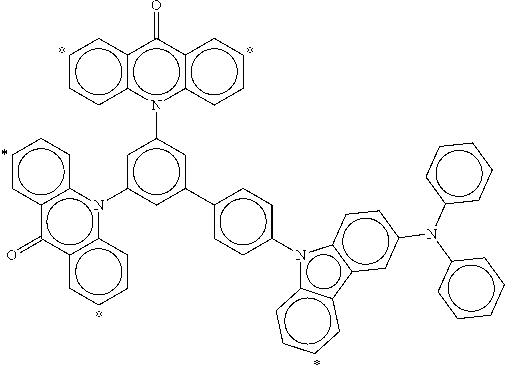 | N136 |
| 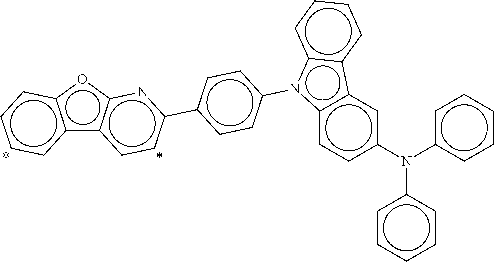 | N137 |
| 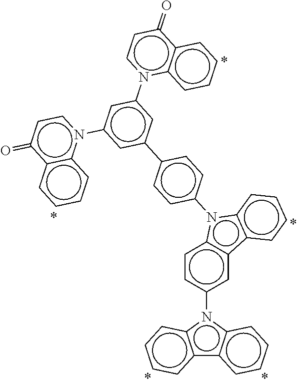 | N138 |
| 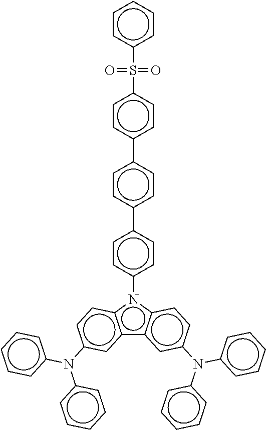 | N139 |
| 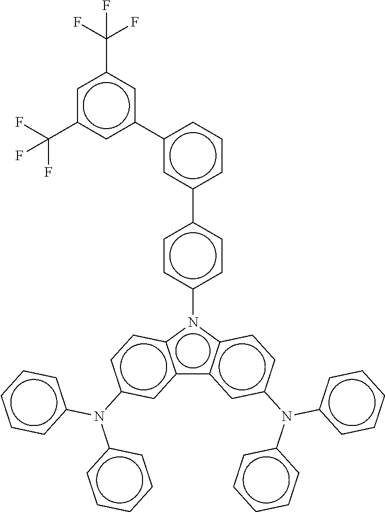 | N140 |
| 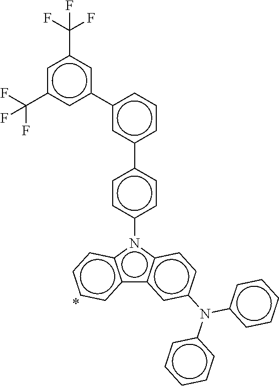 | N141 |
| 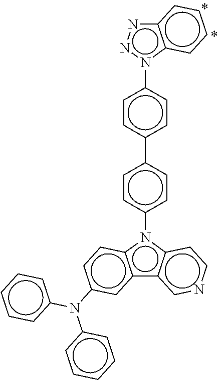 | N142 |
| 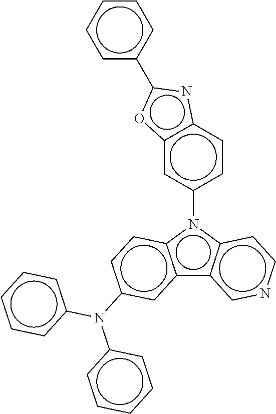 | N143 |
| 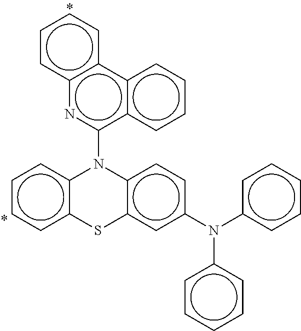 | N144 |
| 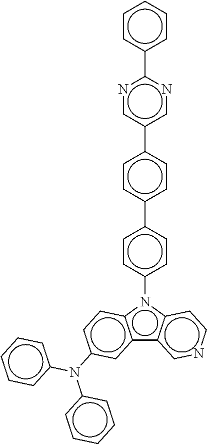 | N145 |
| 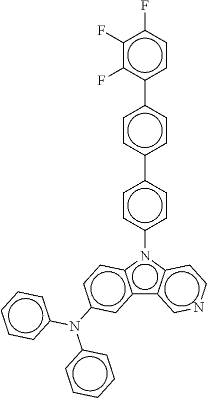 | N146 |
| 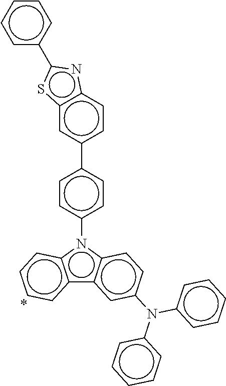 | N147 |
| 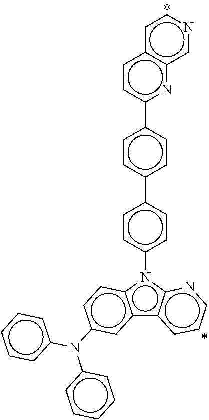 | N148 |
| 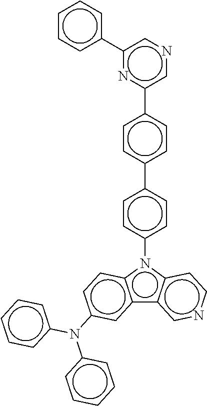 | N149 |
| 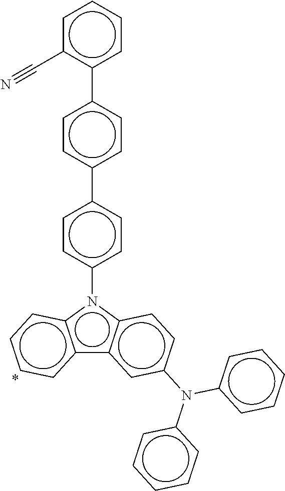 | N150 |
| 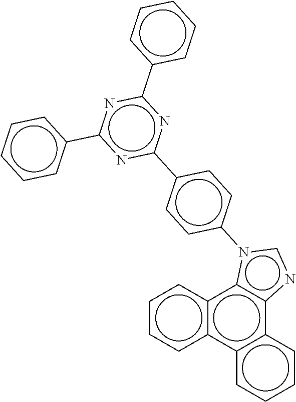 | N151 |

In example embodiments of the fourteenth aspect, the invention is any one compound selected from Table N′. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE N′

| | |
|---|---|
| 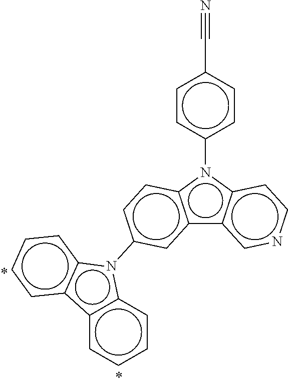 | N44 |
| 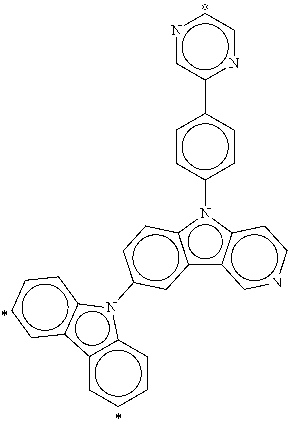 | N34 |
| 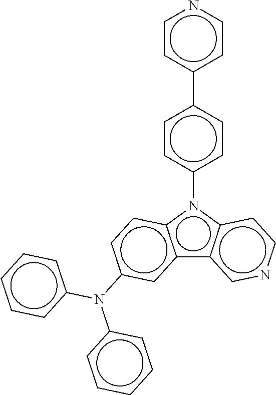 | N59 |
| 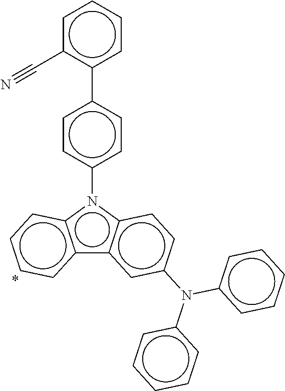 | N17 |
| 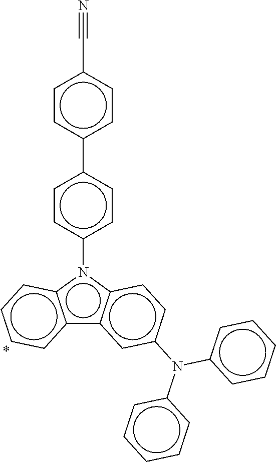 | N55 |
| 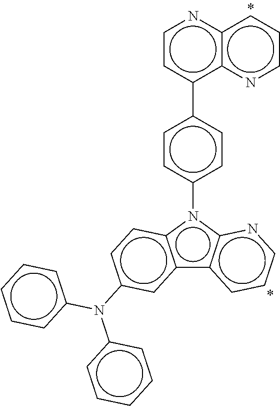 | N14 |
| 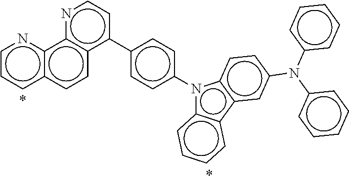 | N68 |

In example embodiments of the fourteenth aspect, the invention is any one compound selected from Table N″. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE N″

| | |
|---|---|
| 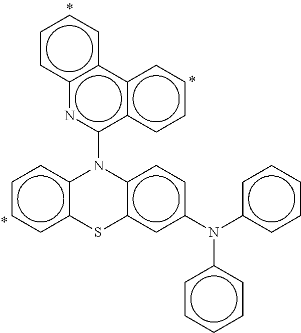 | N144 |
| 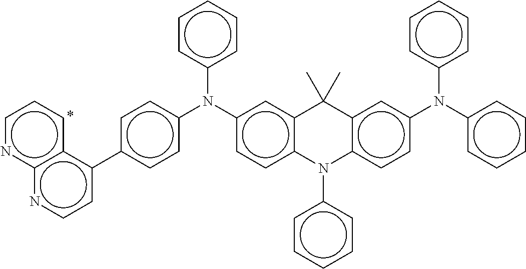 | N128 |

In example embodiments of the fourteenth aspect, the invention is any one compound selected from Table N′″. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE N′′′

| | |
|---|---|
| 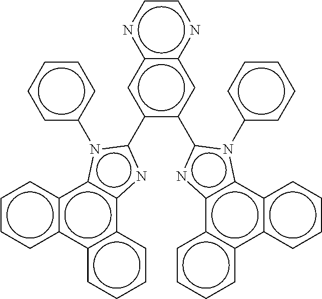 | N53 |
| 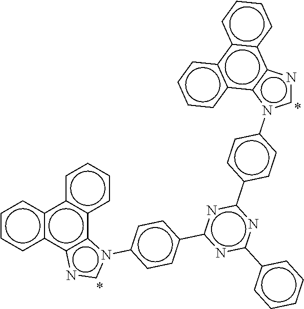 | N92 |
| 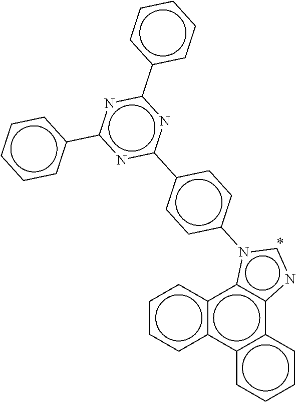 | N151 |

In example embodiments of the fourteenth aspect, the present invention is any one molecule selected from compounds O1 to O123, listed in Table O. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE O

| | |
|---|---|
| 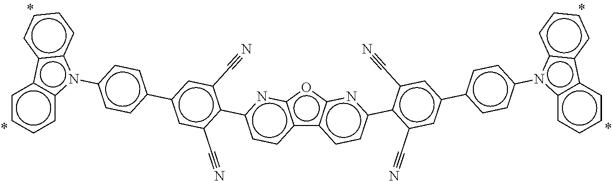 | O1 |
| 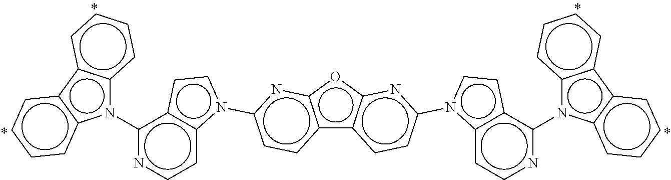 | O2 |
| 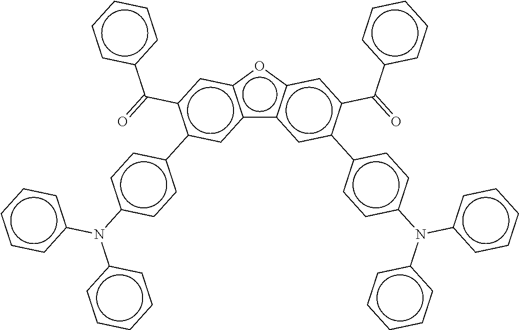 | O3 |
| 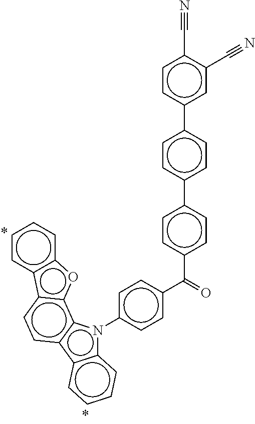 | O4 |
| 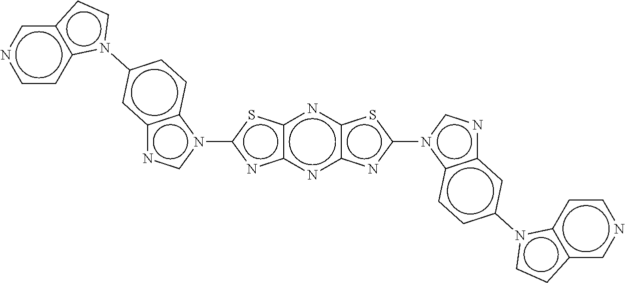 | O5 |
| 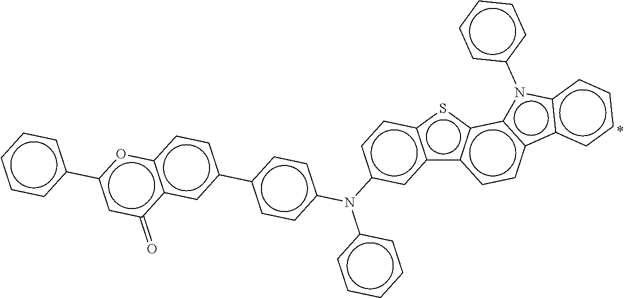 | O6 |
| 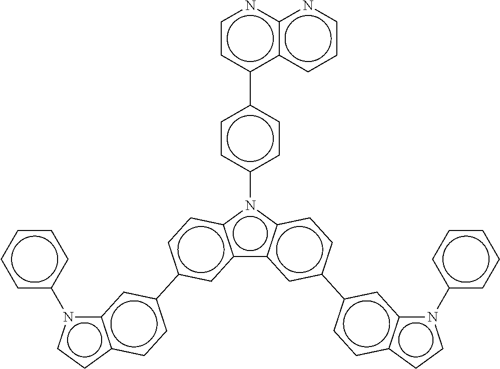 | O7 |
| 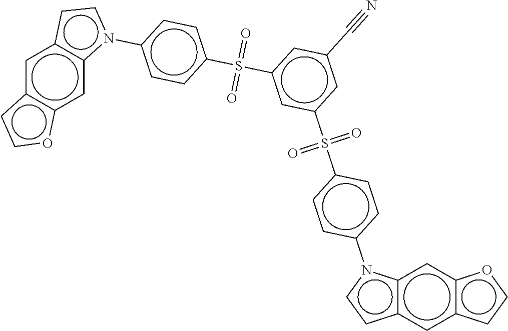 | O8 |
| 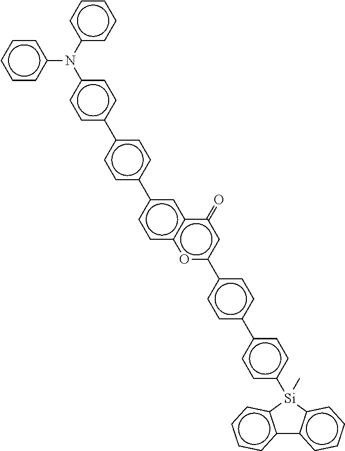 | O9 |
| 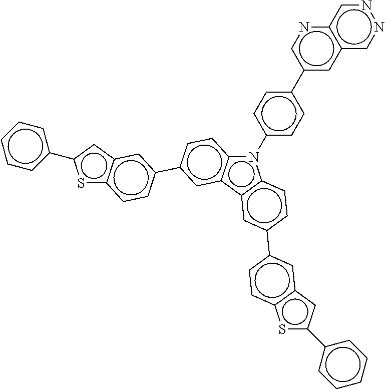 | O10 |
| 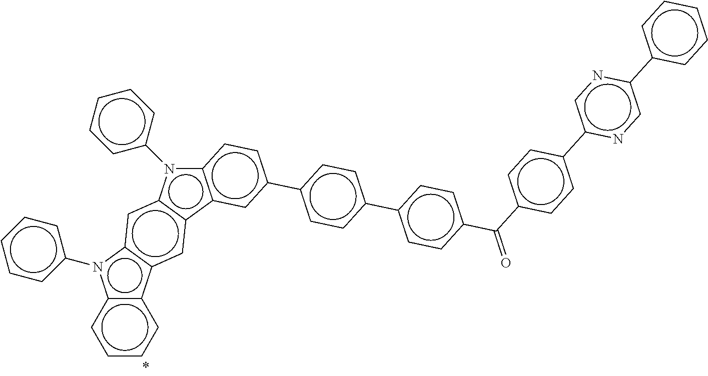 | O11 |
| 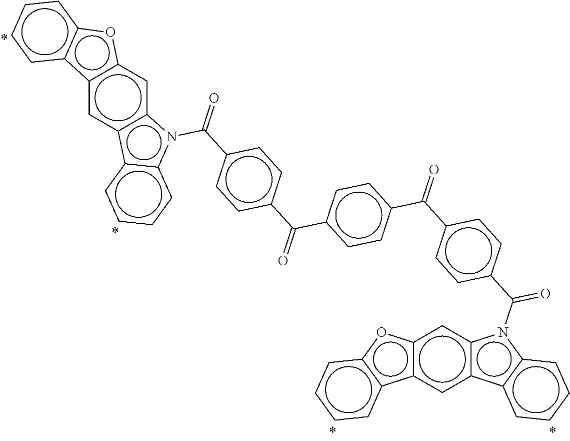 | O12 |
| 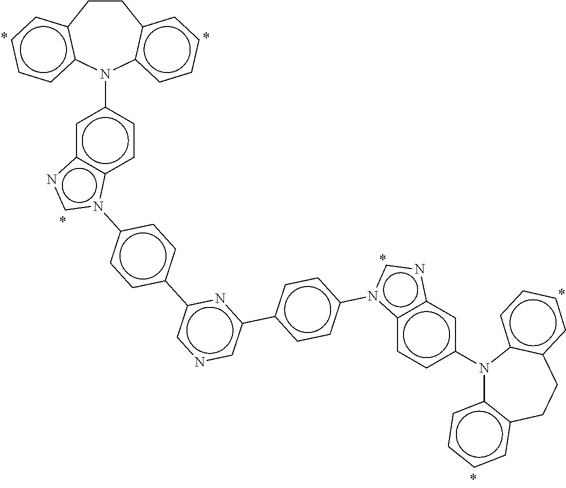 | O13 |
| 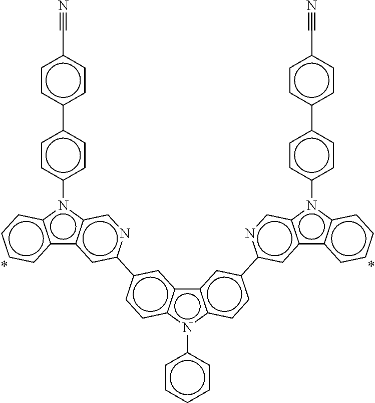 | O14 |
| 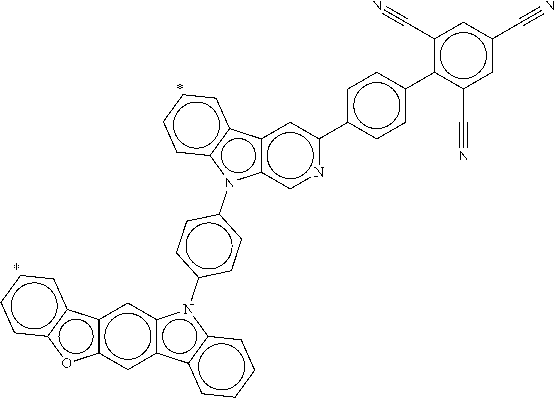 | O15 |
| 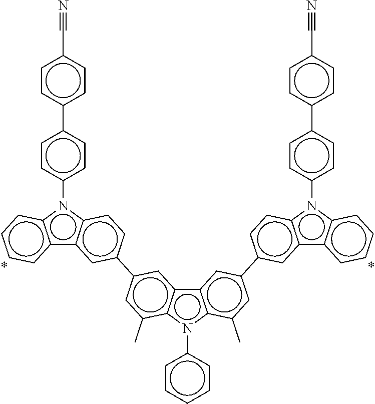 | O16 |
| 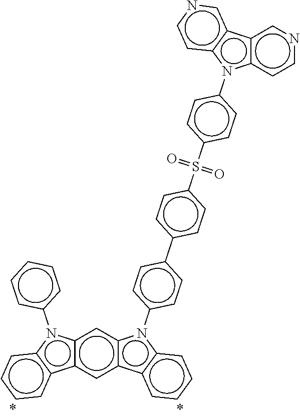 | O17 |
| 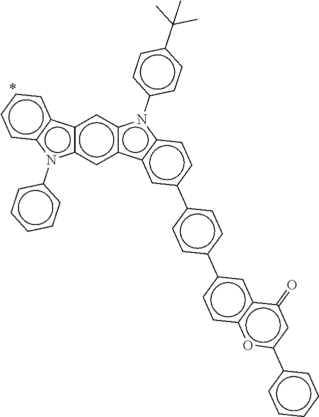 | O18 |
| 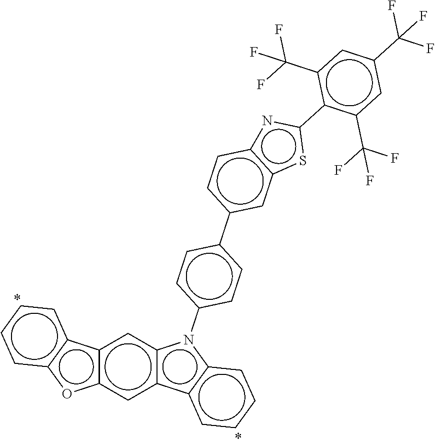 | O19 |
| 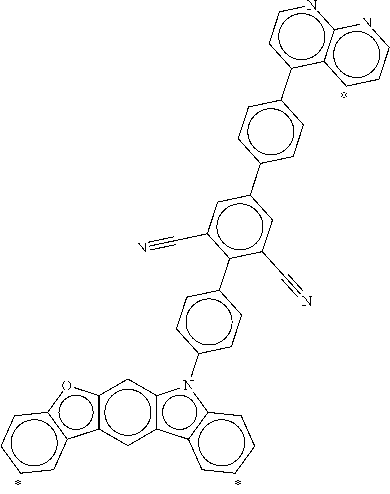 | O20 |
| 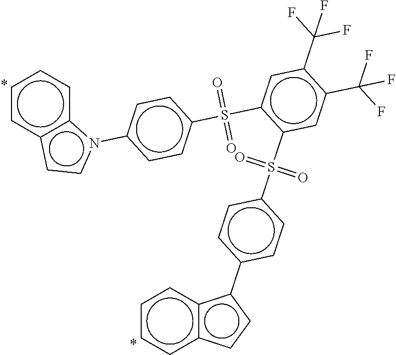 | O21 |
| 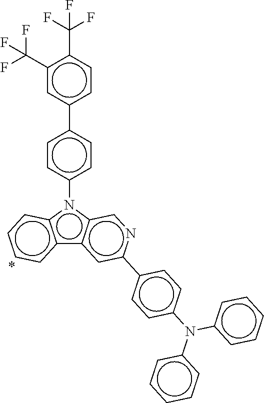 | O22 |
| 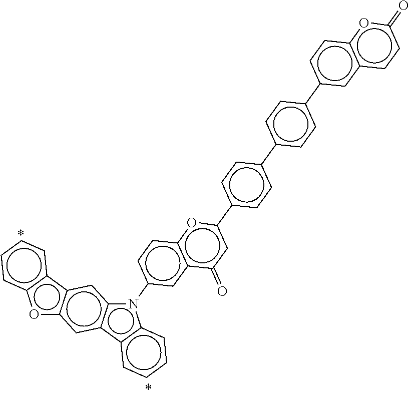 | O23 |
| 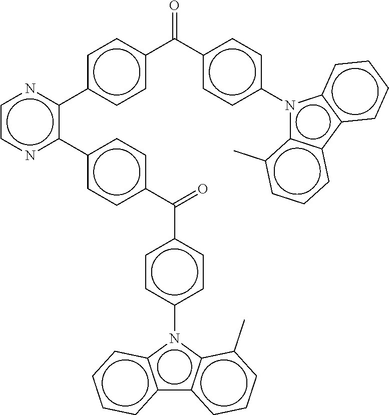 | O24 |
| 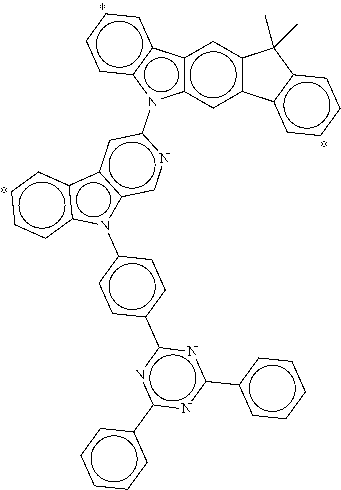 | O25 |
| 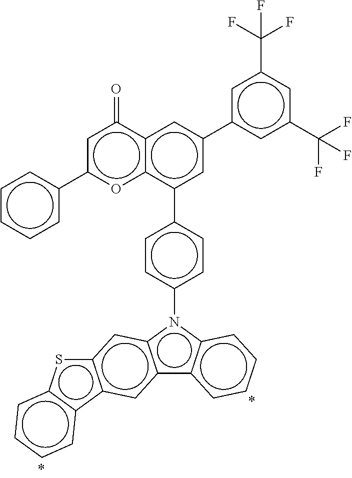 | O26 |
| 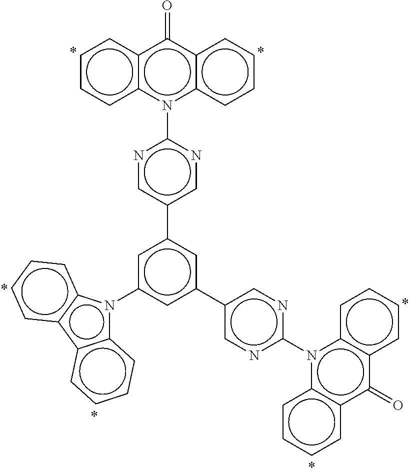 | O27 |
| 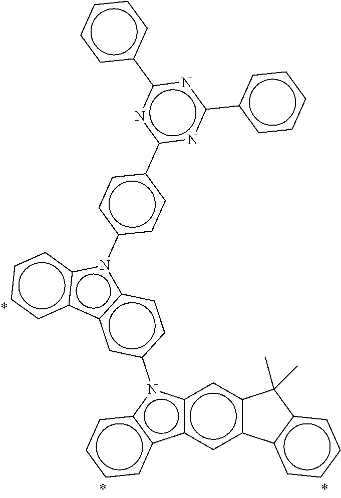 | O28 |
| 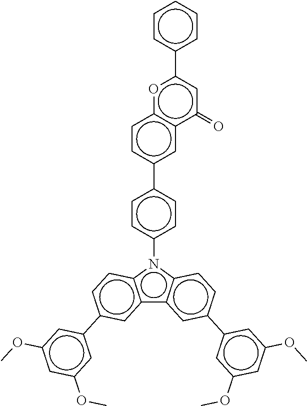 | O29 |
| 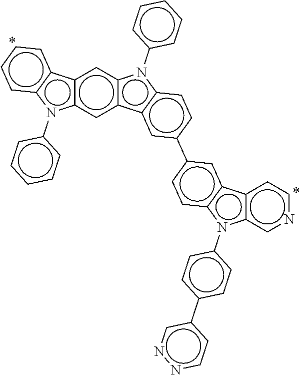 | O30 |
| 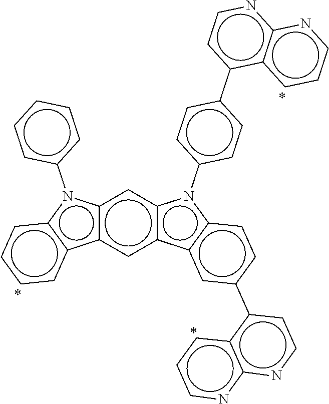 | O31 |
| 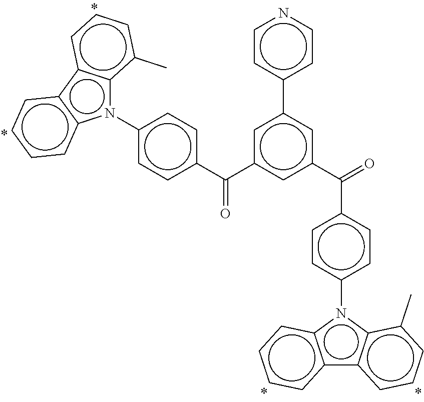 | O32 |
| 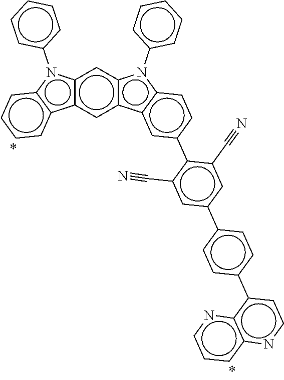 | O33 |
| 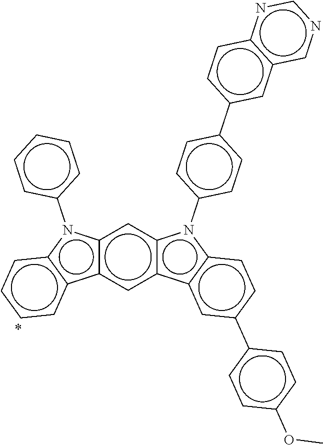 | O34 |
| 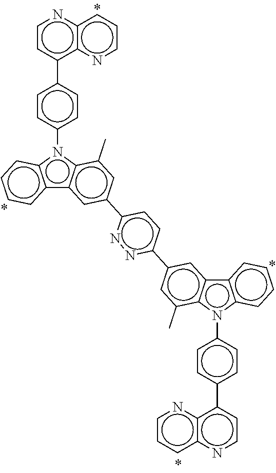 | O35 |
| 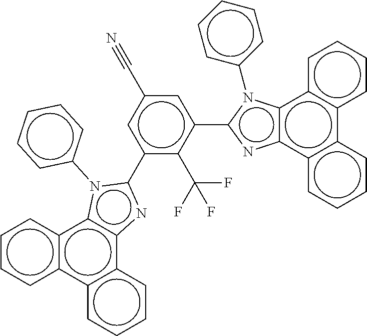 | O36 |
| 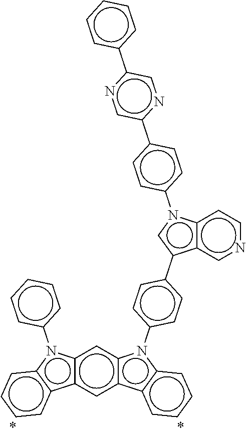 | O37 |
| 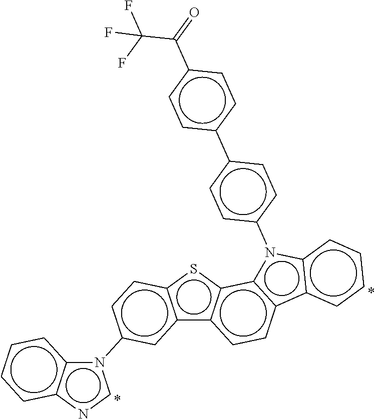 | O38 |
| 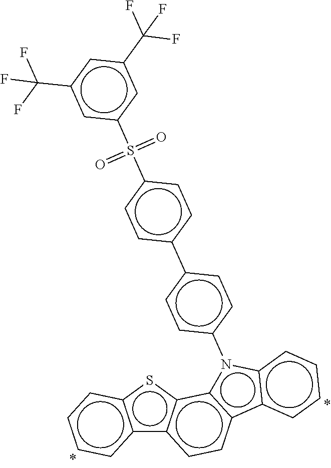 | O39 |
| 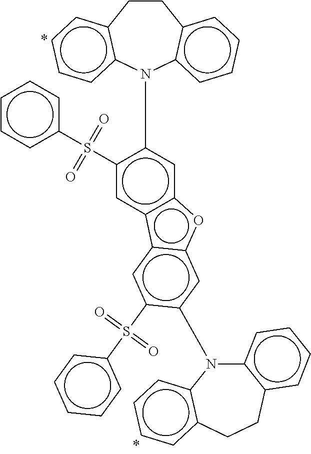 | O40 |
| 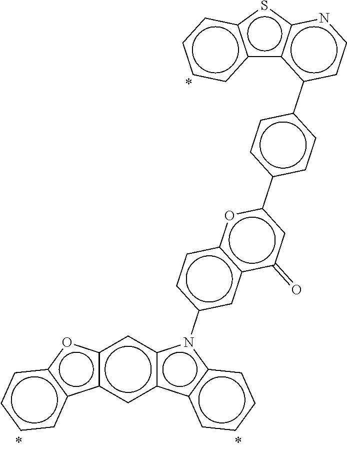 | O41 |
| 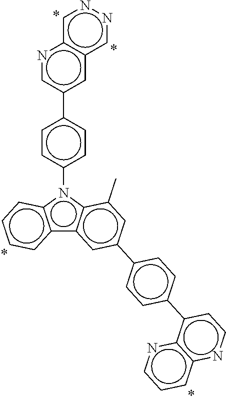 | O42 |
| 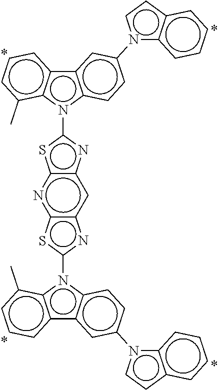 | O43 |
| 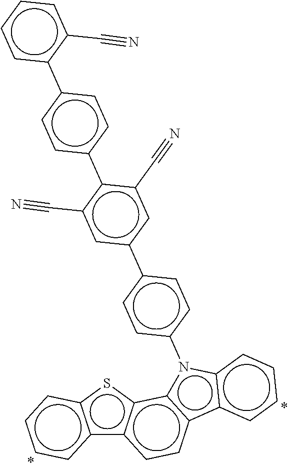 | O44 |
| 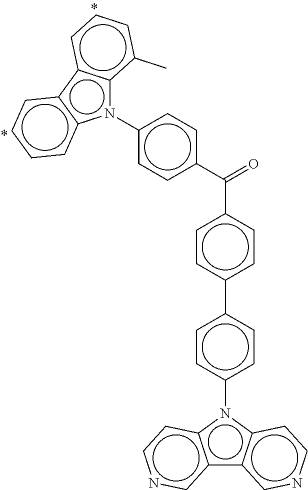 | O45 |
| 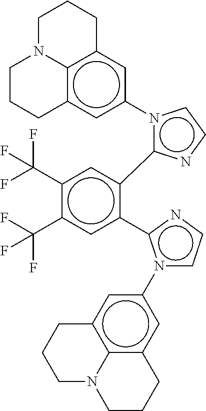 | O46 |
| 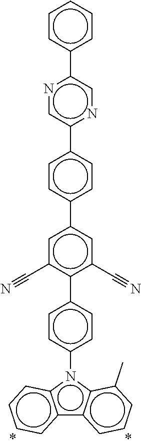 | O47 |
| 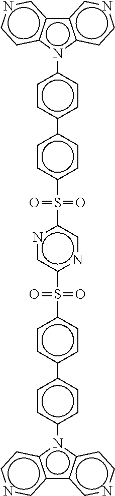 | O48 |
| 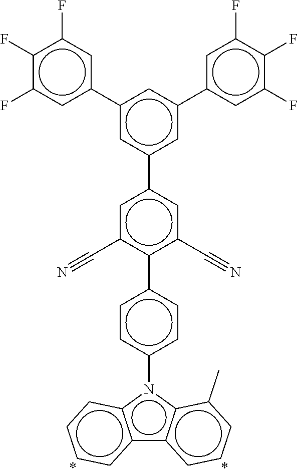 | O49 |
| 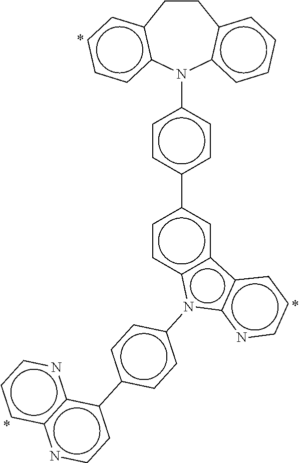 | O50 |
| 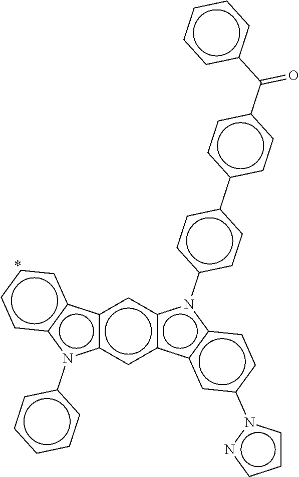 | O51 |
| 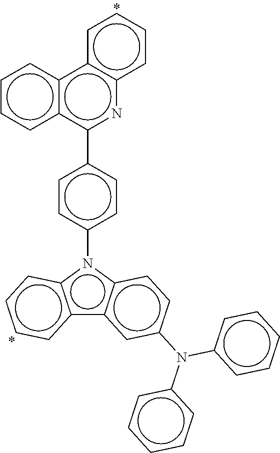 | O52 |
| 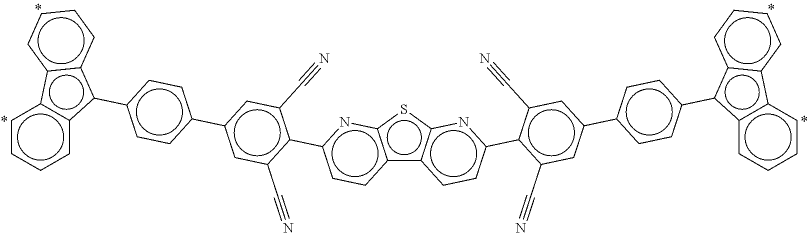 | O53 |
| 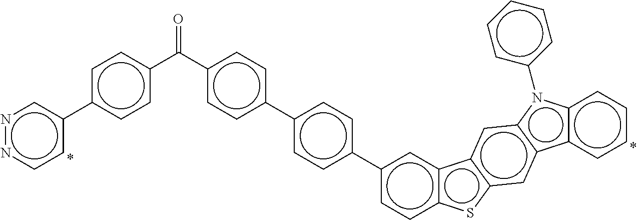 | O54 |
| 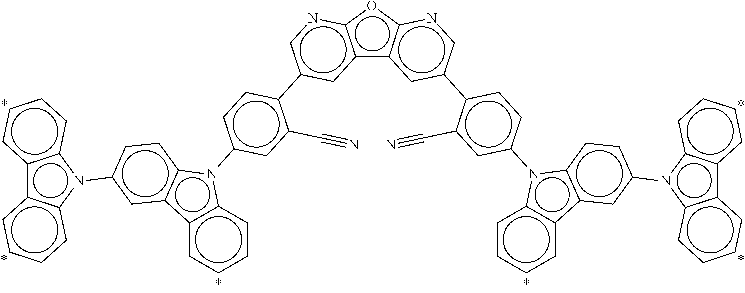 | O55 |
| 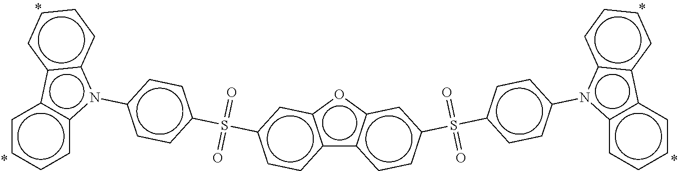 | O56 |
| 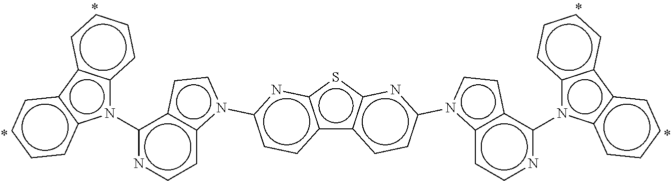 | O57 |
| 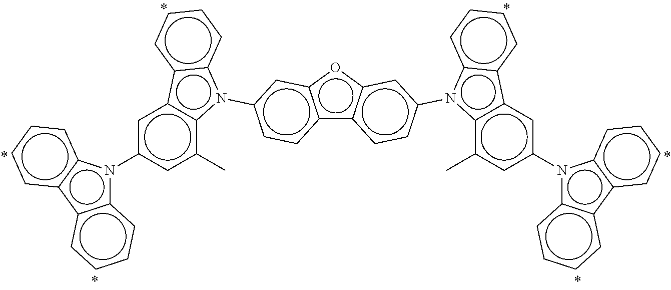 | O58 |
| 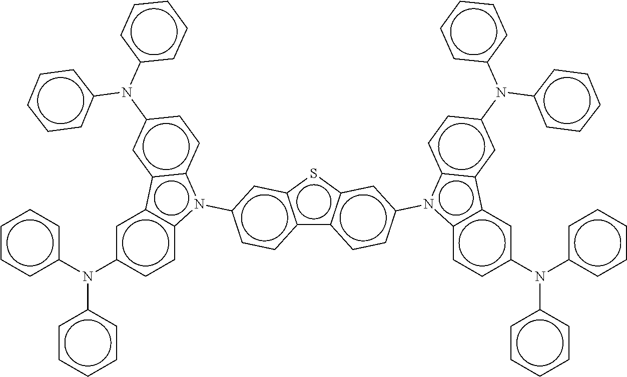 | O59 |
| 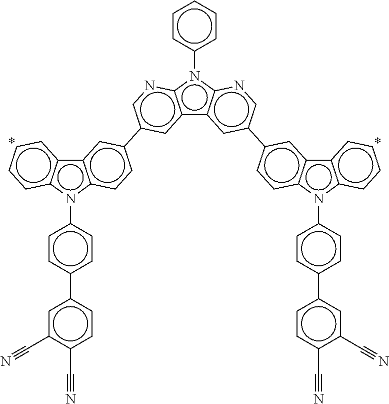 | O60 |
| 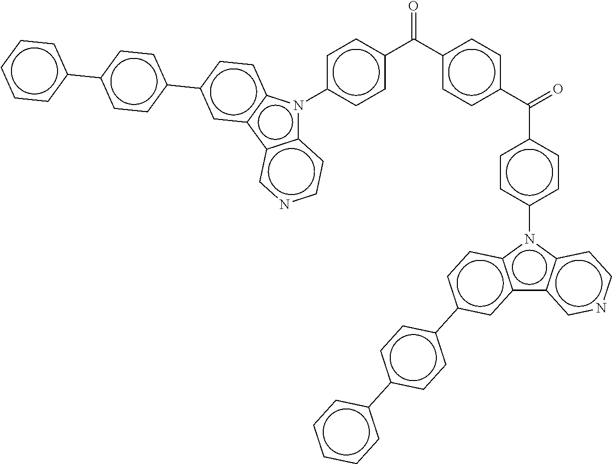 | O61 |
| 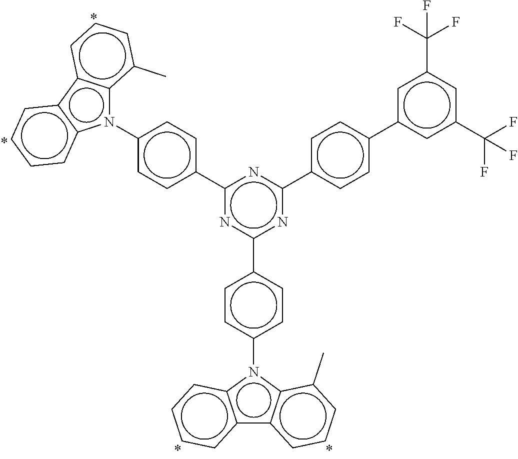 | O62 |
| 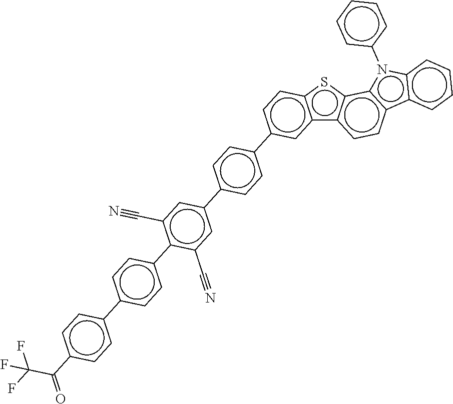 | O63 |
| 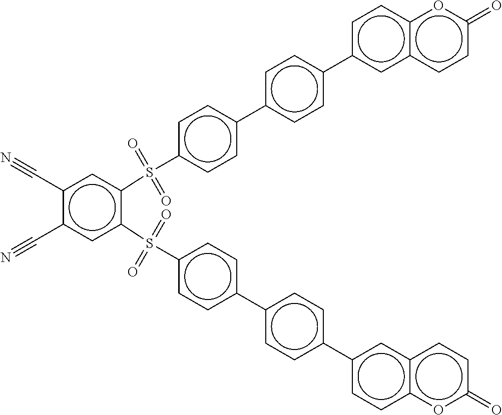 | O64 |
| 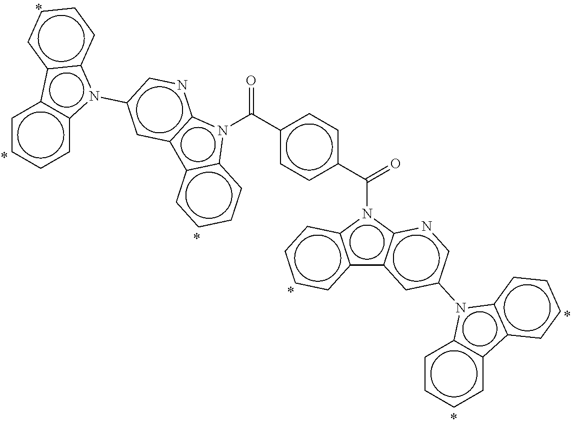 | O65 |
| 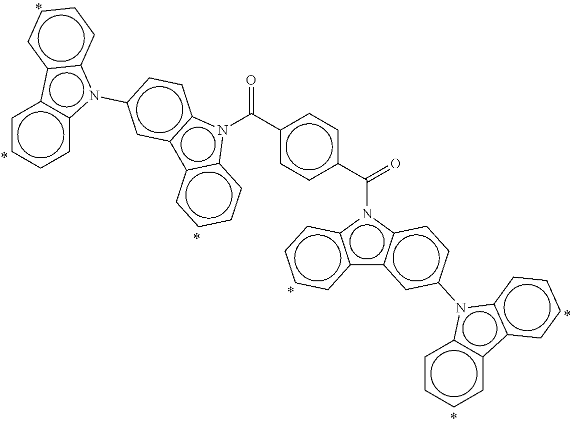 | O66 |
| 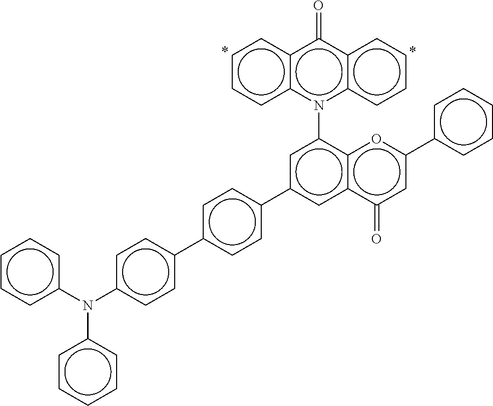 | O67 |
| 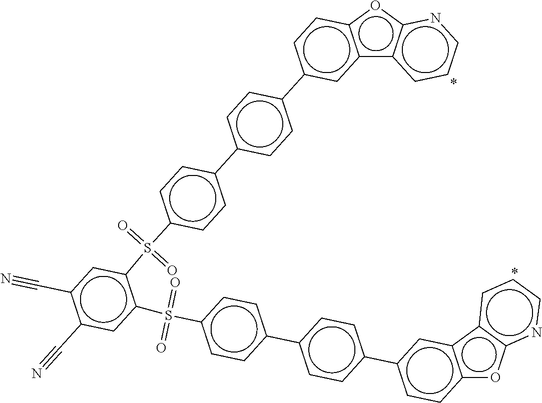 | O68 |
| 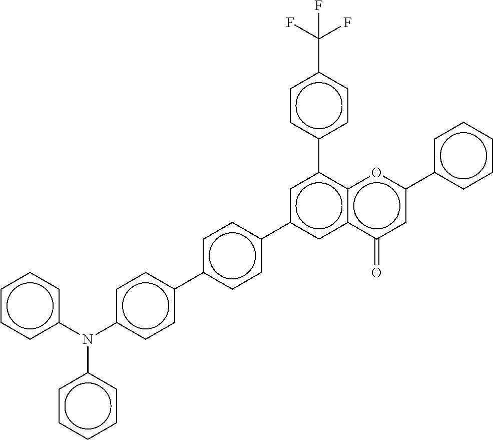 | O69 |
| 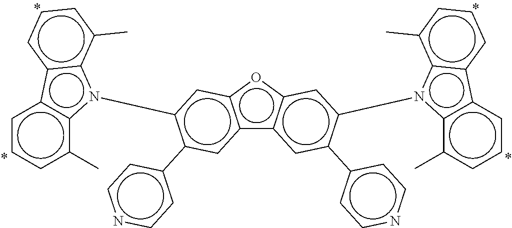 | O70 |
| 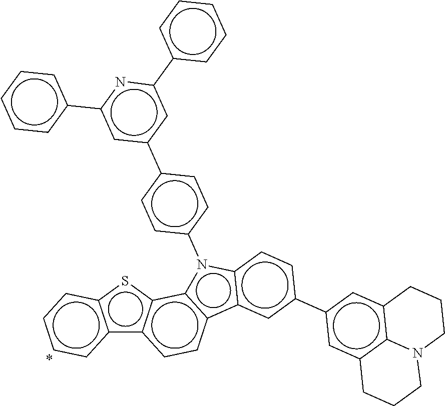 | O71 |
| 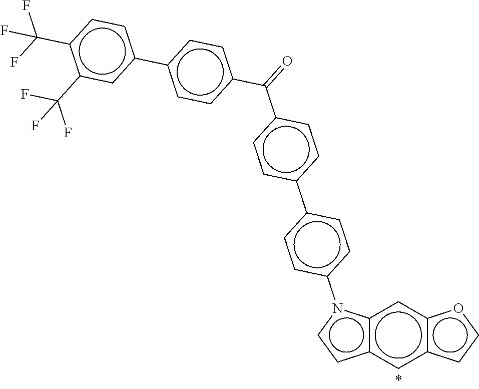 | O72 |
| 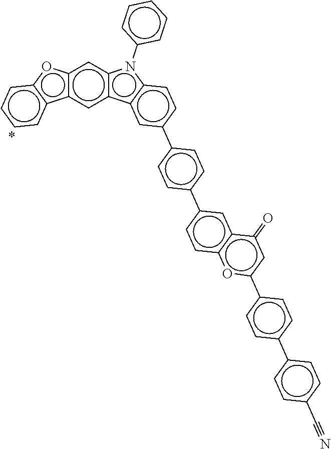 | O73 |
| 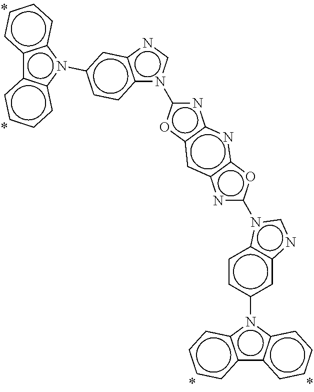 | O74 |
| 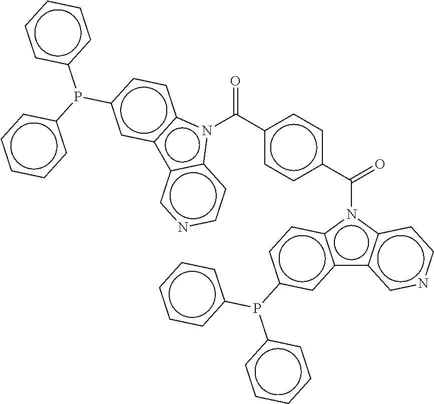 | O75 |
| 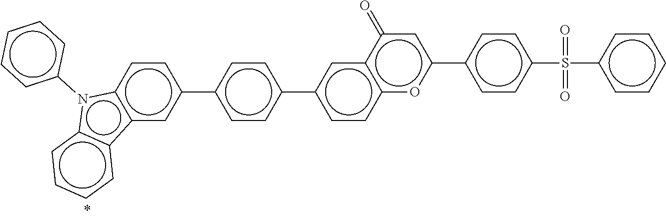 | O76 |
| 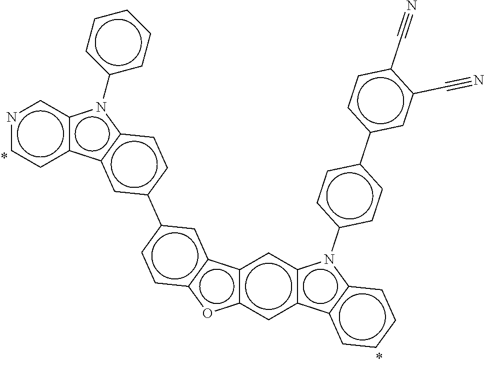 | O77 |
| 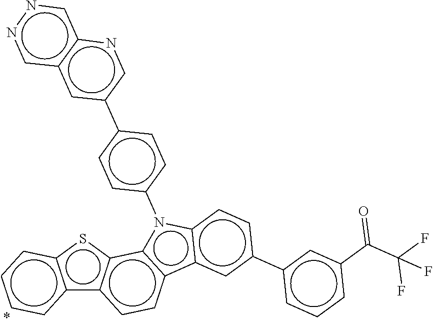 | O78 |
| 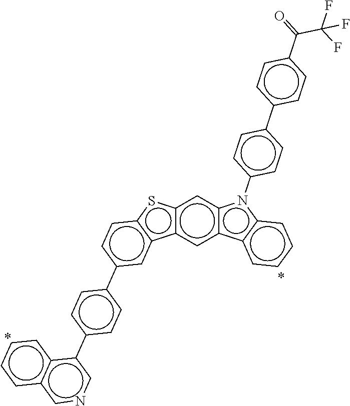 | O79 |
| 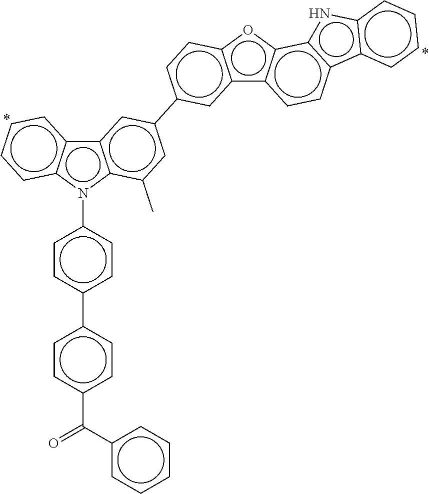 | O80 |
| 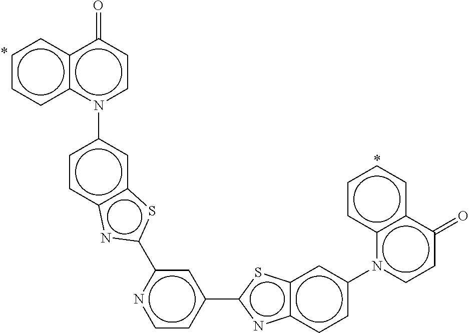 | O81 |
| 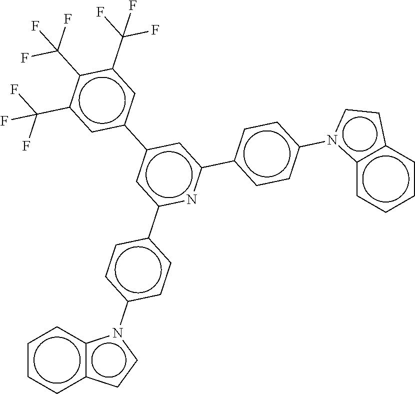 | O82 |
| 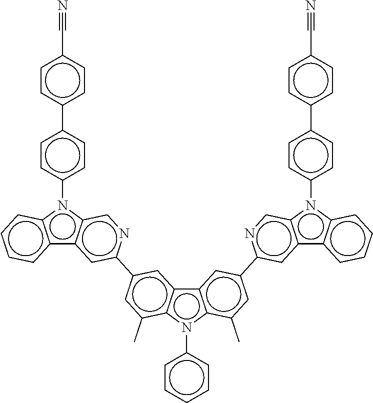 | O83 |
| 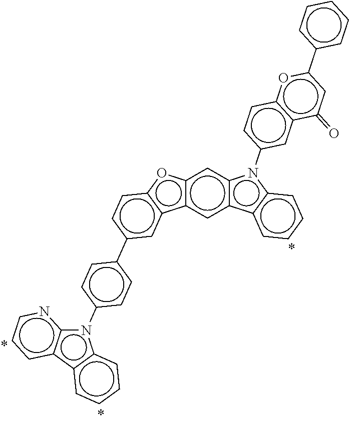 | O84 |
| 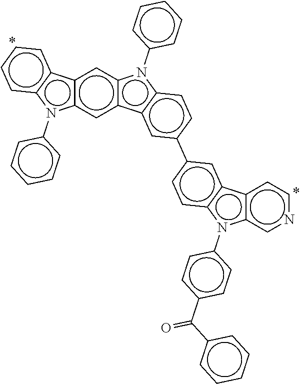 | O85 |
| 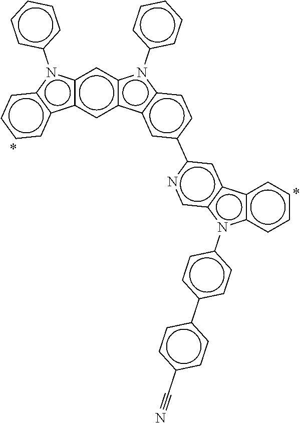 | O86 |
| 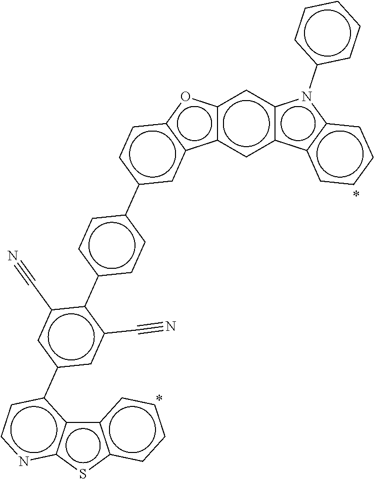 | O87 |
| 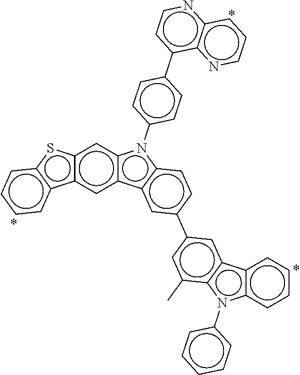 | O88 |
| 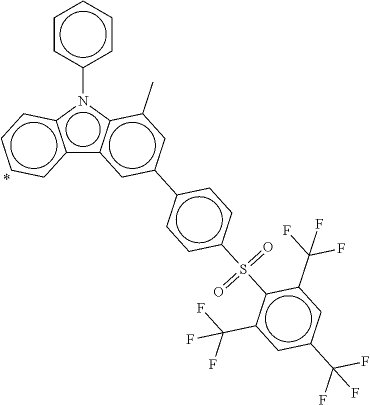 | O89 |
| 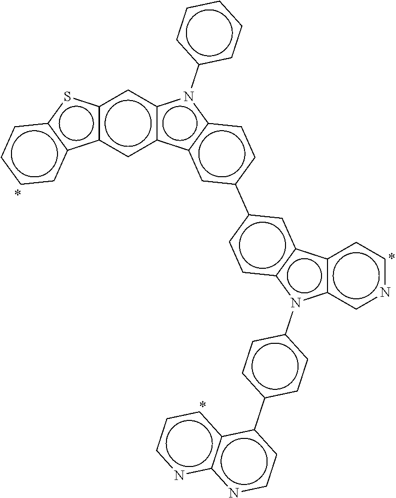 | O90 |
| 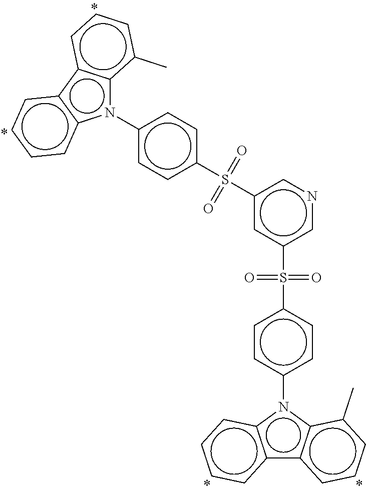 | O91 |
| 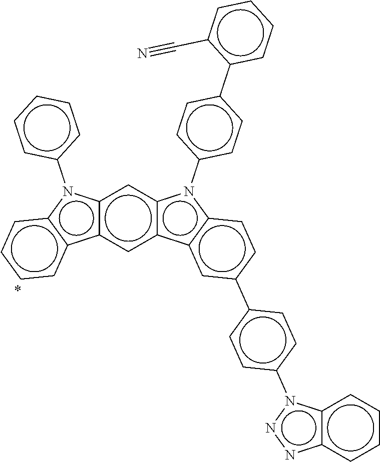 | O92 |
| 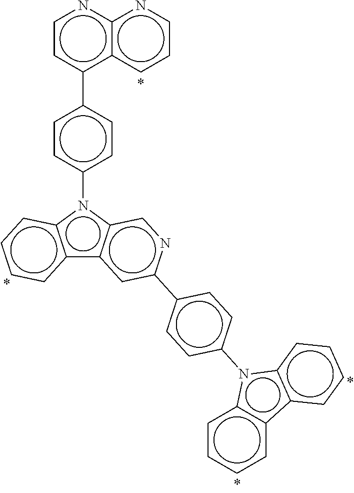 | O93 |
| 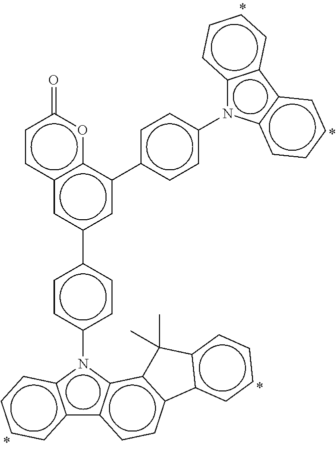 | O94 |
| 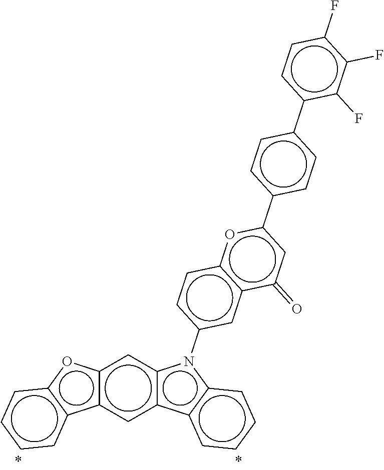 | O95 |
| 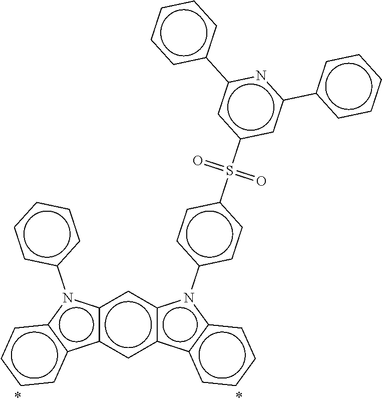 | O96 |
| 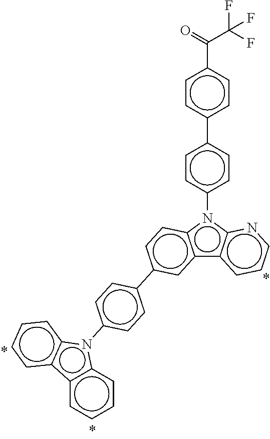 | O97 |
| 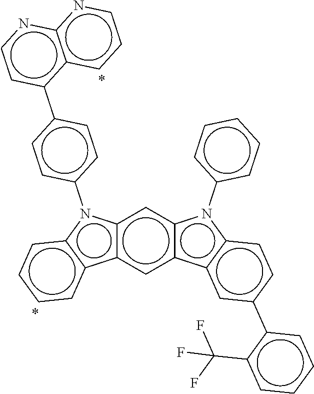 | O98 |
| 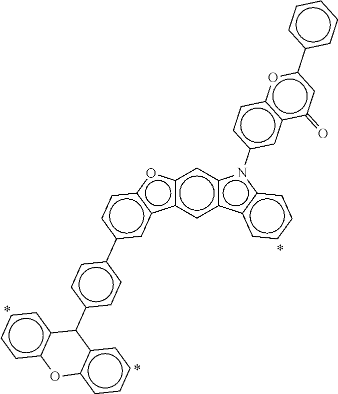 | O99 |
| 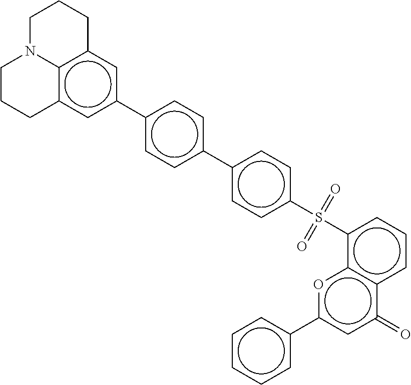 | O100 |
| 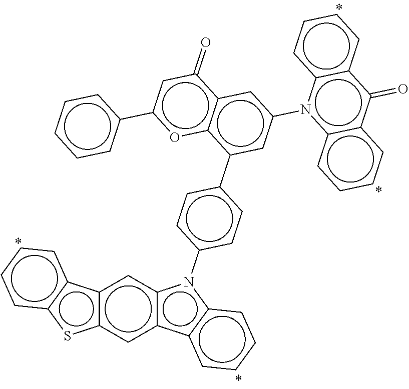 | O101 |
| 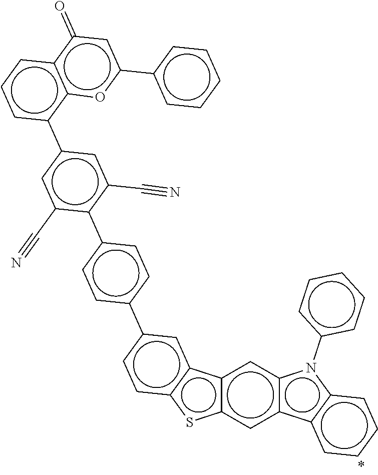 | O102 |
| 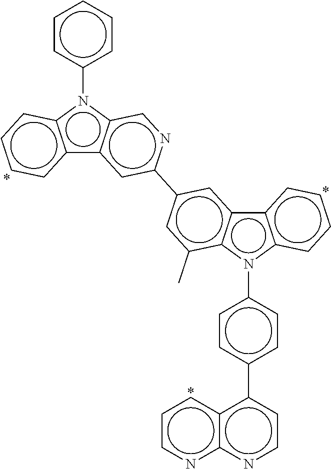 | O103 |
| 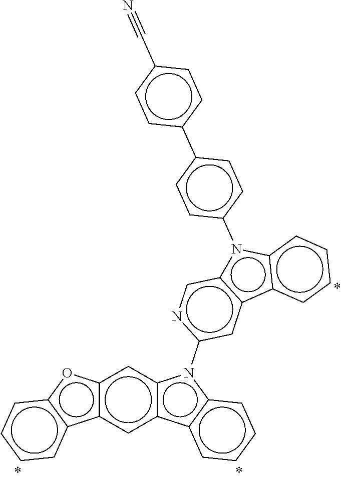 | O104 |
| 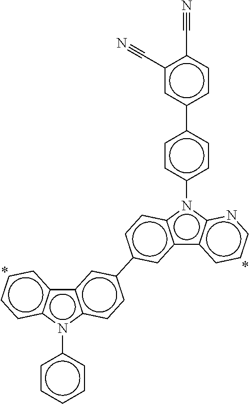 | O105 |
| 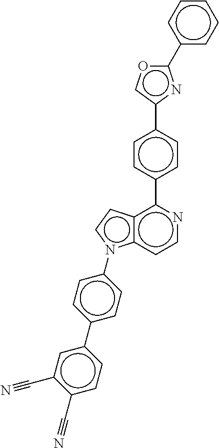 | O106 |
| 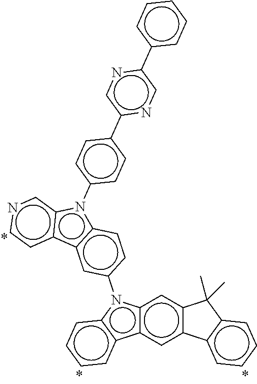 | O107 |
| 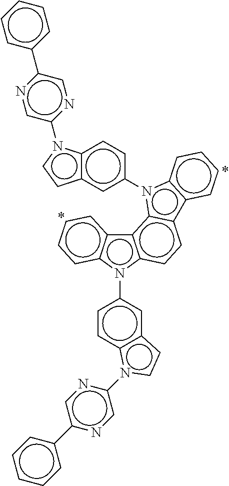 | O108 |
| 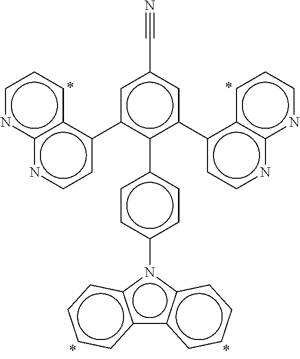 | O109 |
| 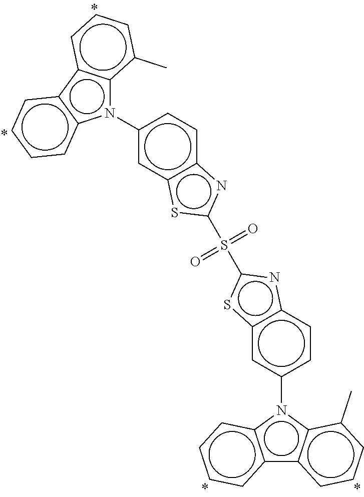 | O110 |
| 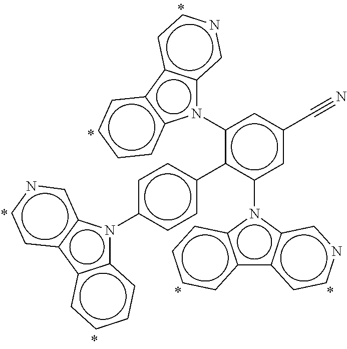 | O111 |
| 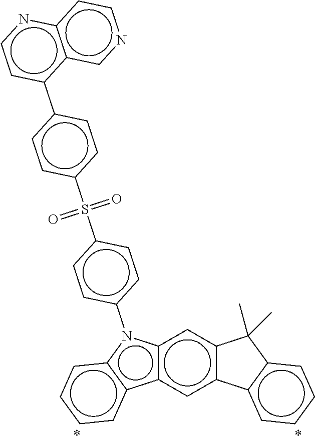 | O112 |
| 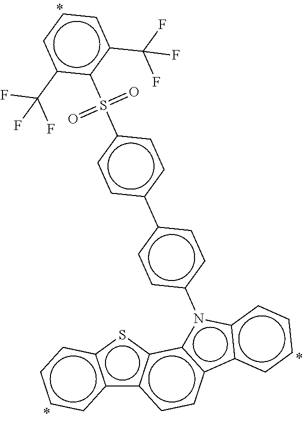 | O113 |
| 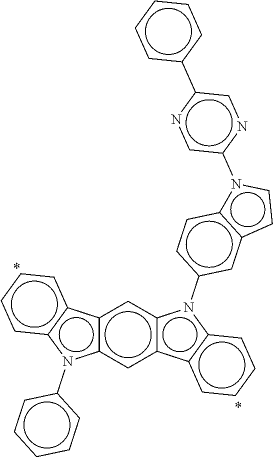 | O114 |
| 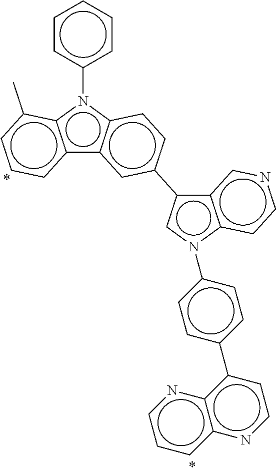 | O115 |
| 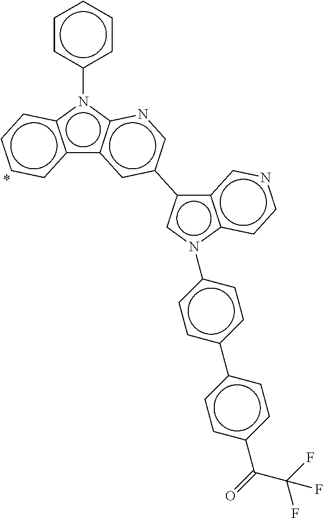 | O116 |
| 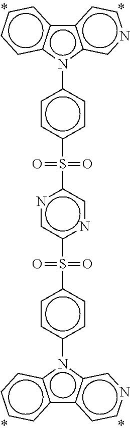 | O117 |
| 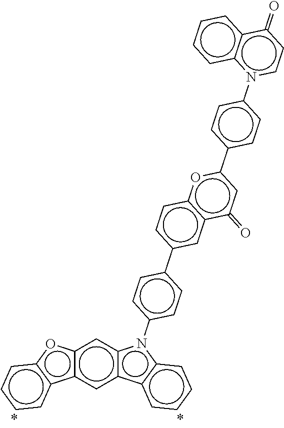 | O118 |
| 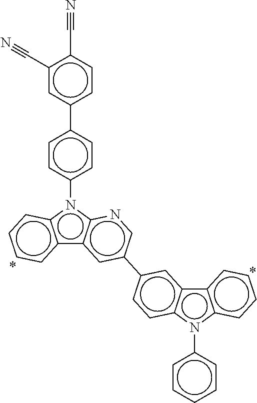 | O119 |
| 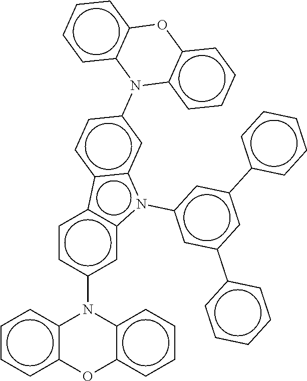 | O120 |
| 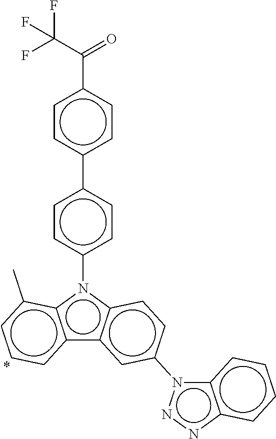 | O121 |
| 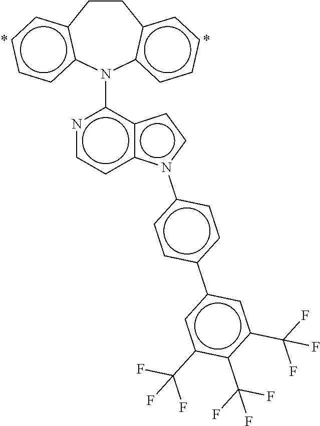 | O122 |
| 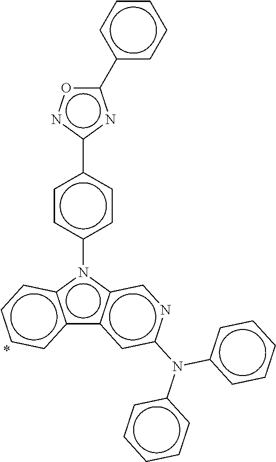 | O123 |

In example embodiments of the fourteenth aspect, the present invention is a molecule selected from compounds Q1 to Q12, listed in Table Q. The variables and substitution patterns on the molecule may be selected as described above with respect to the fourteenth aspect. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE Q

| | |
|---|---|
| 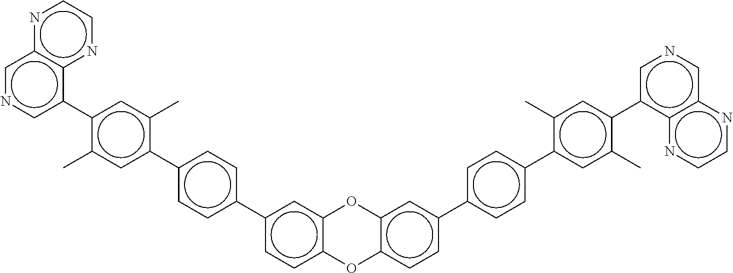 | Q1 |
| 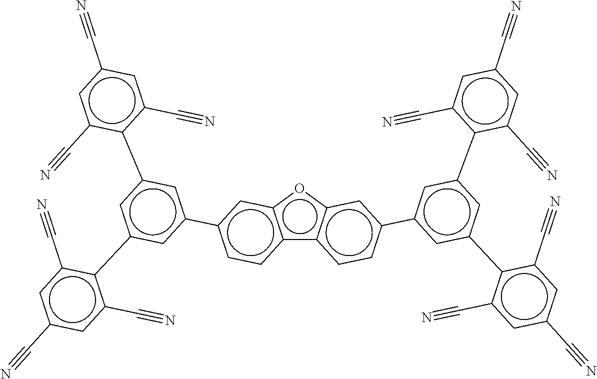 | Q2 |
| 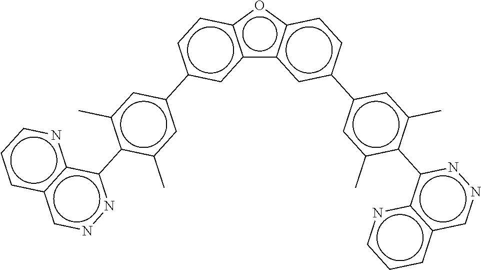 | Q3 |
| 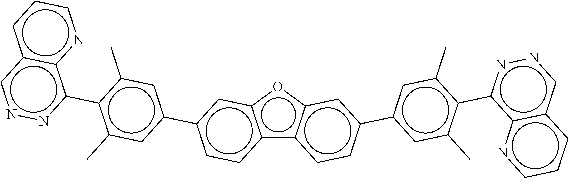 | Q4 |
| 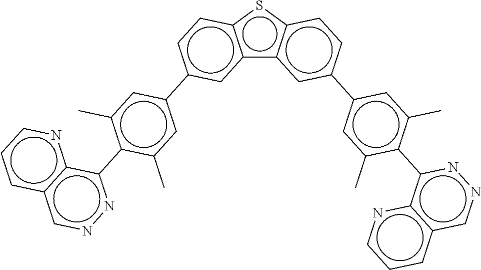 | Q5 |
| 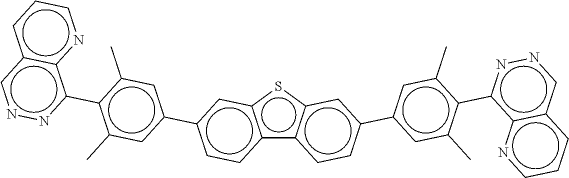 | Q6 |
| 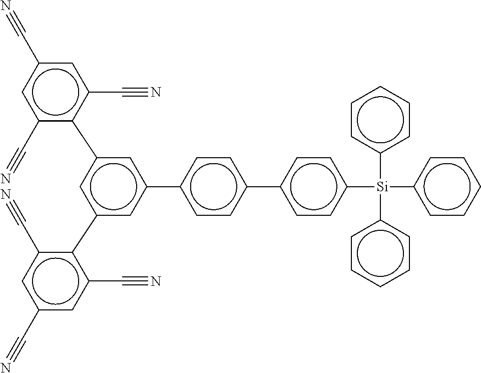 | Q7 |
| 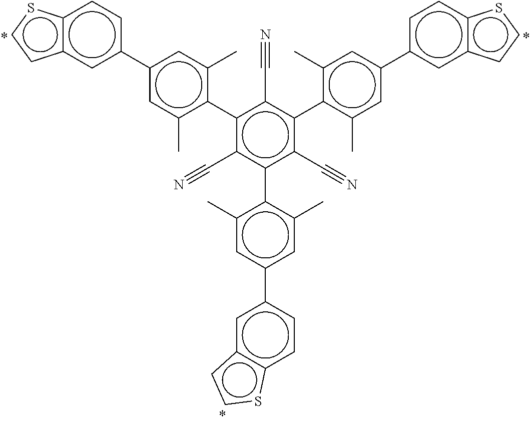 | Q8 |
| 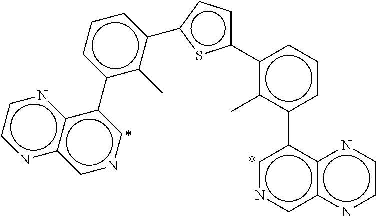 | Q9 |
| 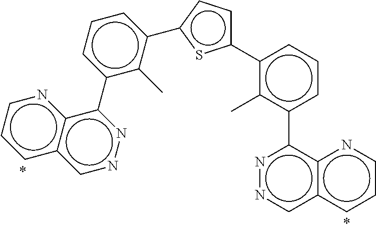 | Q10 |
| 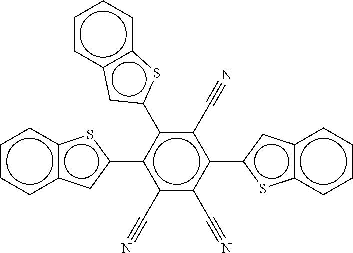 | Q11 |
| 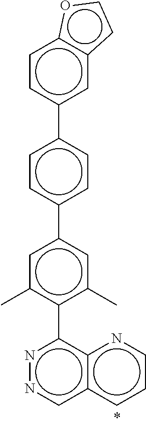 | Q12 |

In example embodiments of the fourteenth aspect, the present invention is the molecule of Table Q′. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE Q′

| | |
|---|---|
| 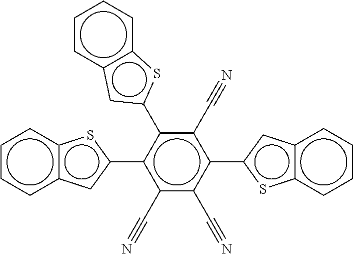 | Q11 |

In example embodiments of the fourteenth aspect, the present invention is any one molecule selected from compounds B1 to B35, listed in Table B. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE B

| | |
|---|---|
| 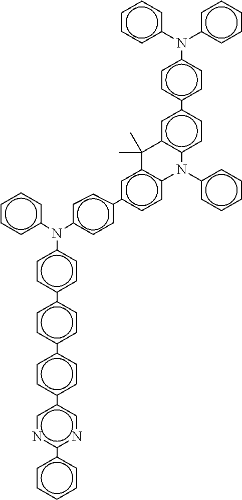 | B1 |
| 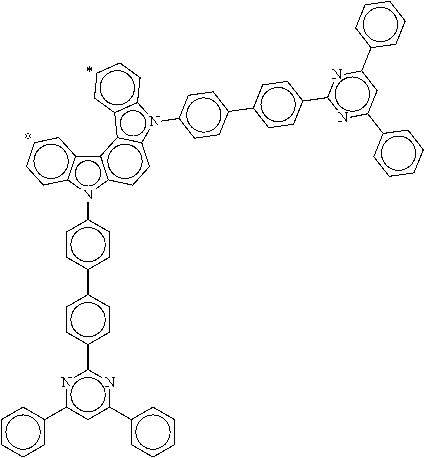 | B2 |
| 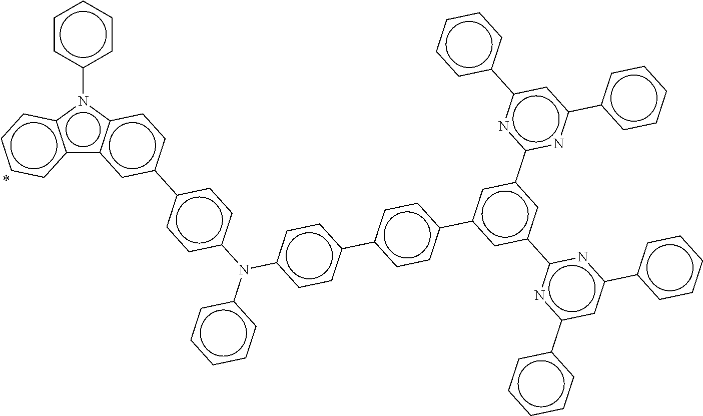 | B3 |
| 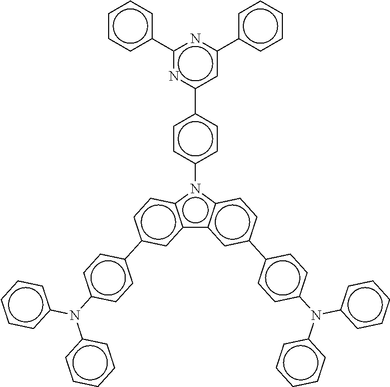 | B4 |
| 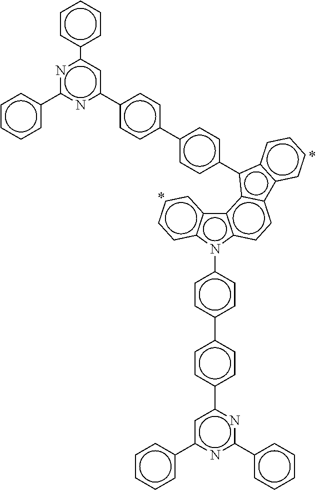 | B5 |
| 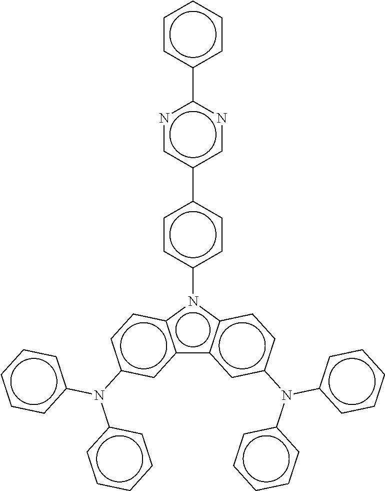 | B6 |
| 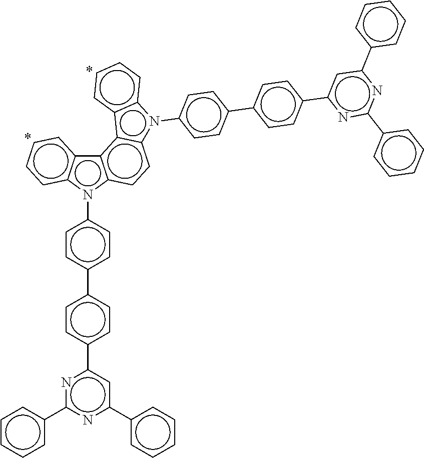 | B7 |
| 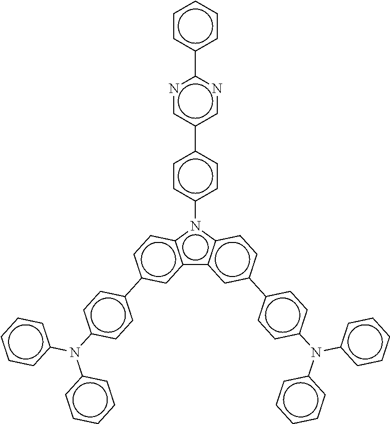 | B8 |
| 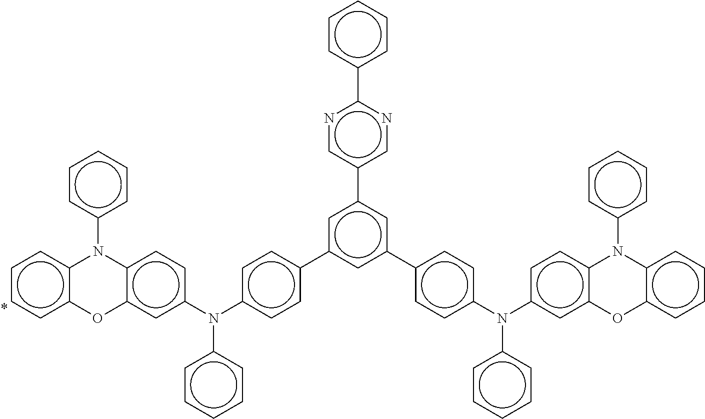 | B9 |
| 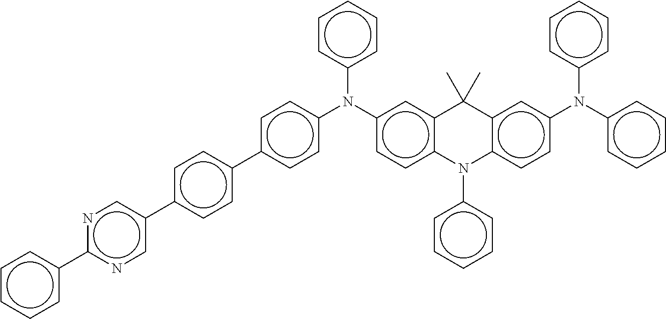 | B10 |
| 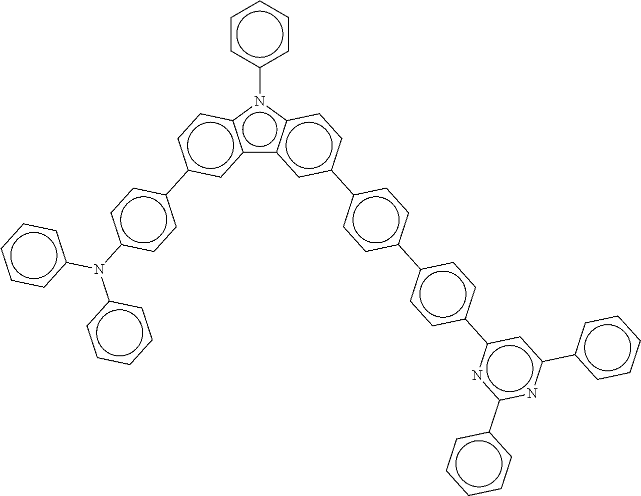 | B11 |
| 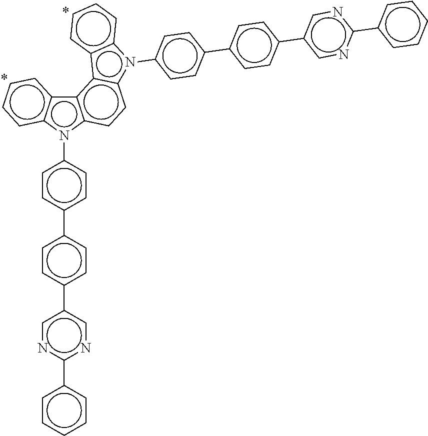 | B12 |
| 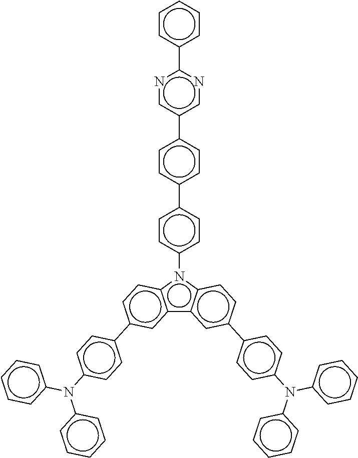 | B13 |
| 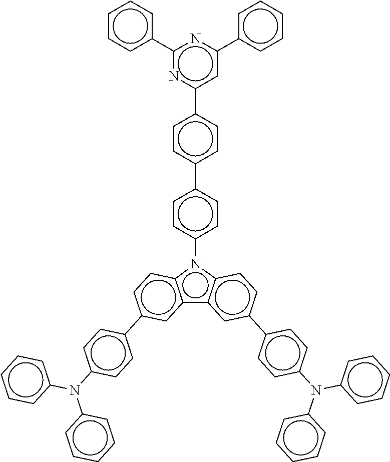 | B14 |
| 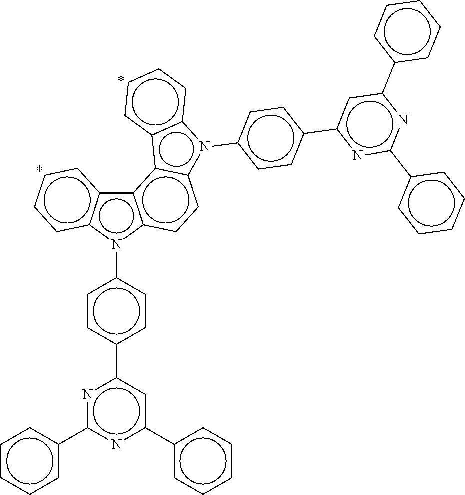 | B15 |
| 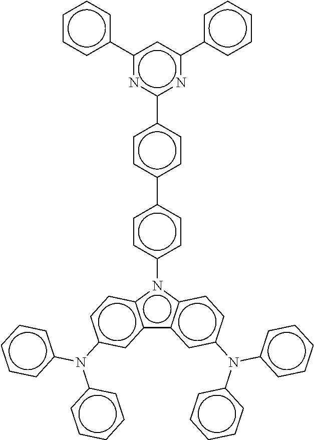 | B16 |
| 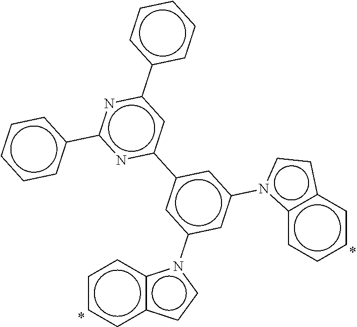 | B17 |
| 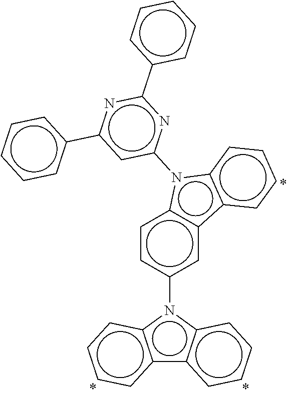 | B18 |
| 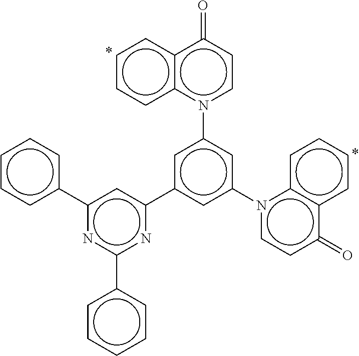 | B19 |
| 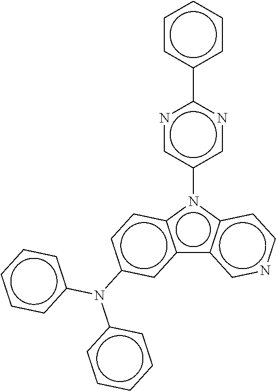 | B20 |
| 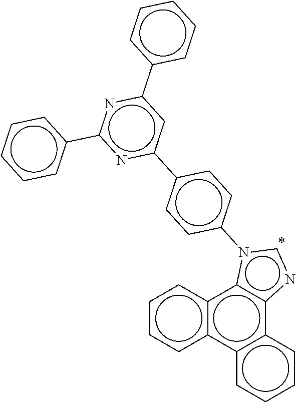 | B21 |
| 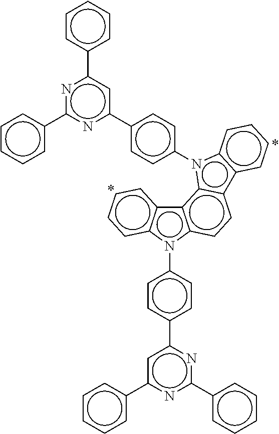 | B22 |
| 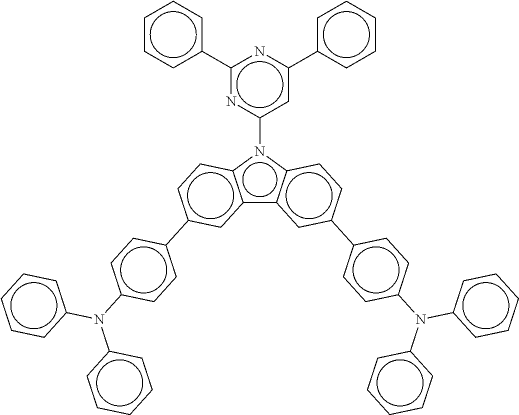 | B23 |
| 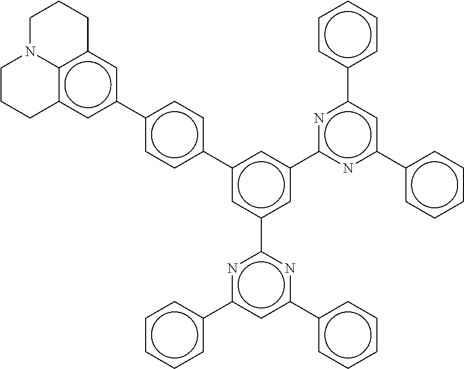 | B24 |
| 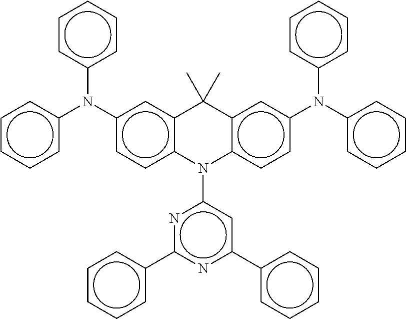 | B25 |
| 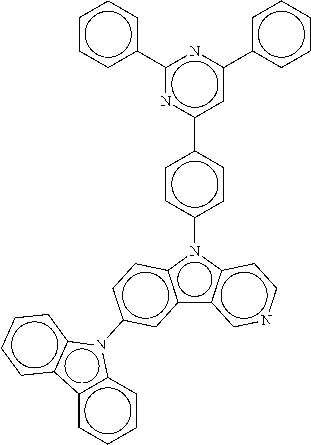 | B26 |
| 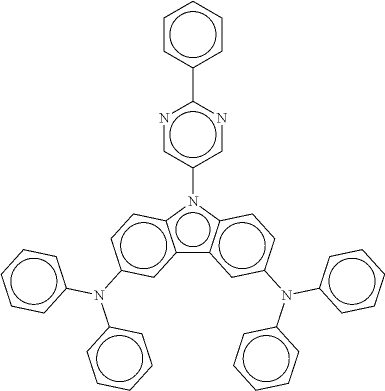 | B27 |
| 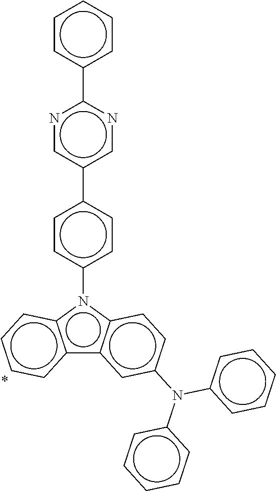 | B28 |
| 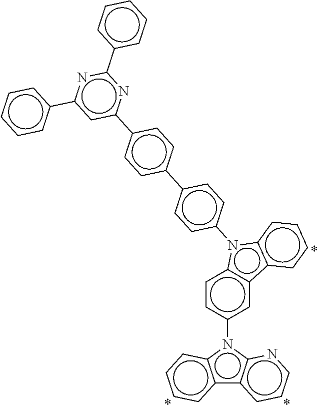 | B29 |
| 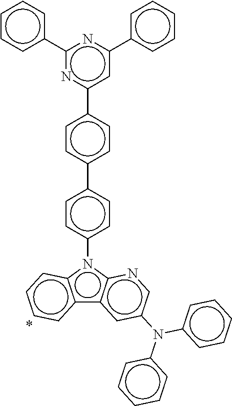 | B30 |
| 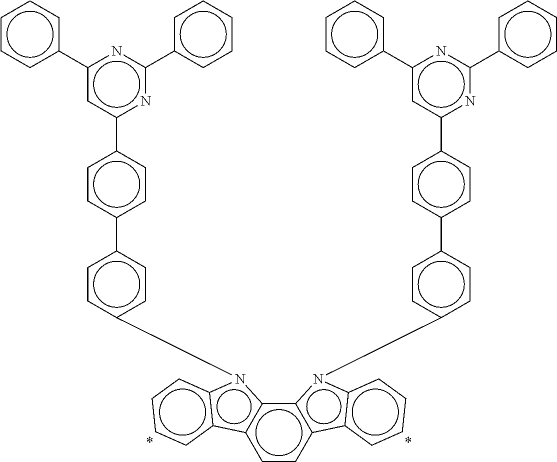 | B31 |
| 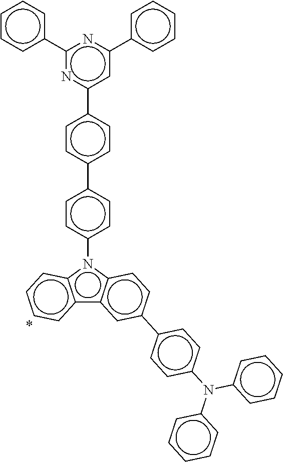 | B32 |
| 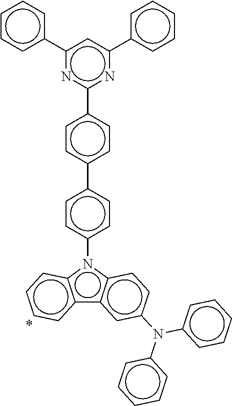 | B33 |
| 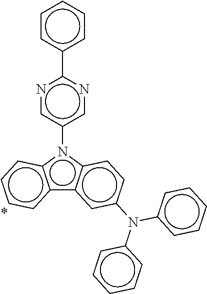 | B34 |
| 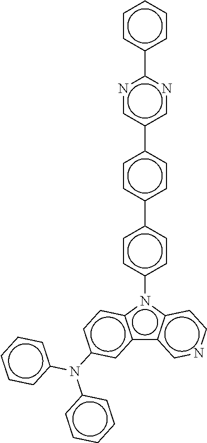 | B35 |

In example embodiments of the fourteenth aspect, the present invention is a molecule selected from compounds R1 to R108, listed in Table R. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE R

| | |
|---|---|
| 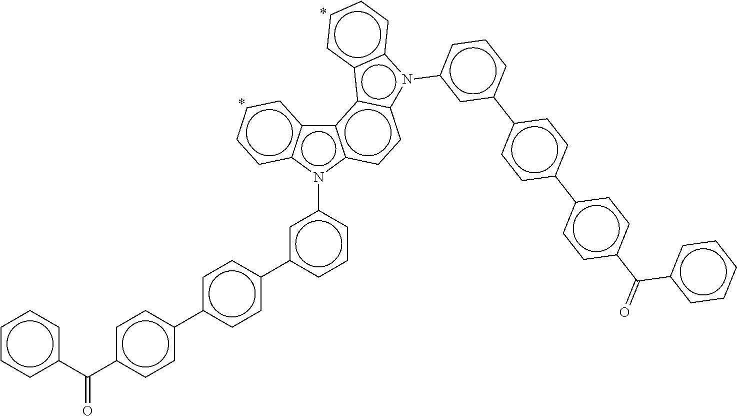 | R1 |
| 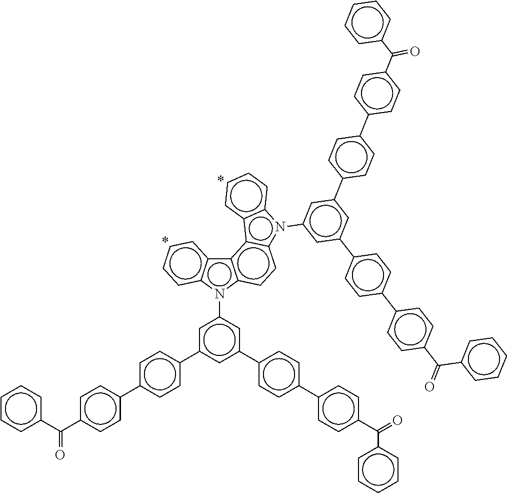 | R2 |
| 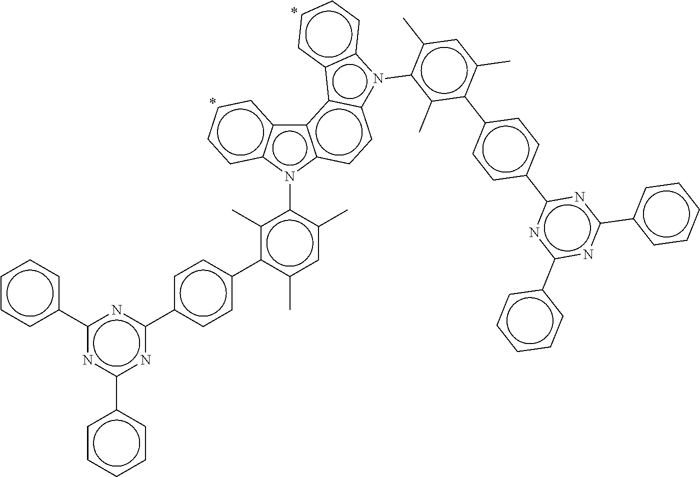 | R3 |
| 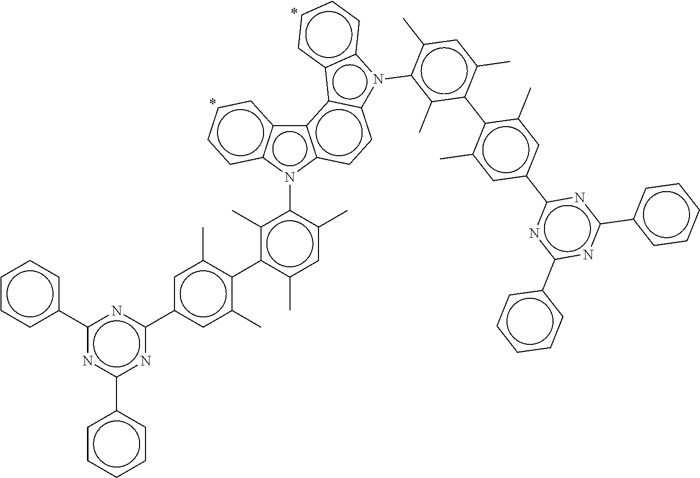 | R4 |
| 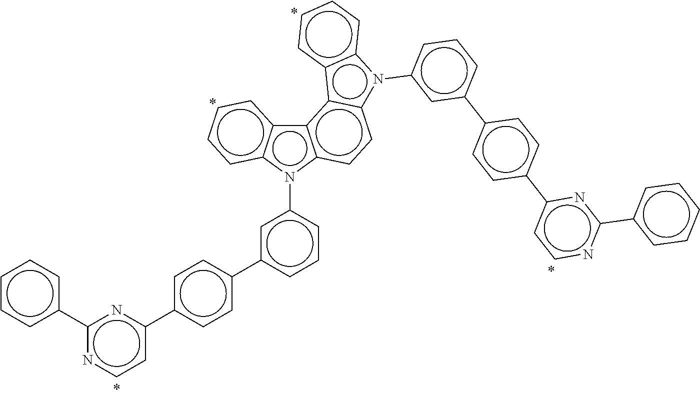 | R5 |
| 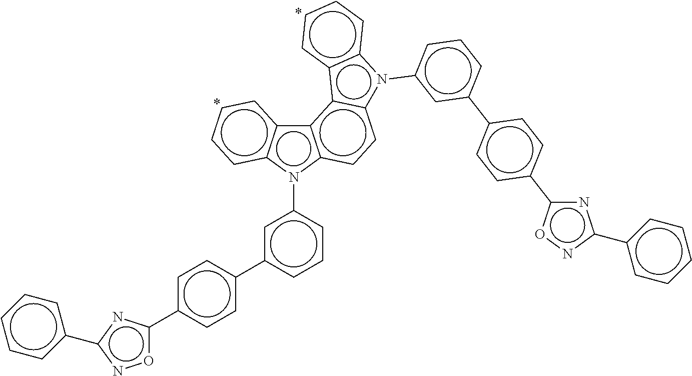 | R6 |
| 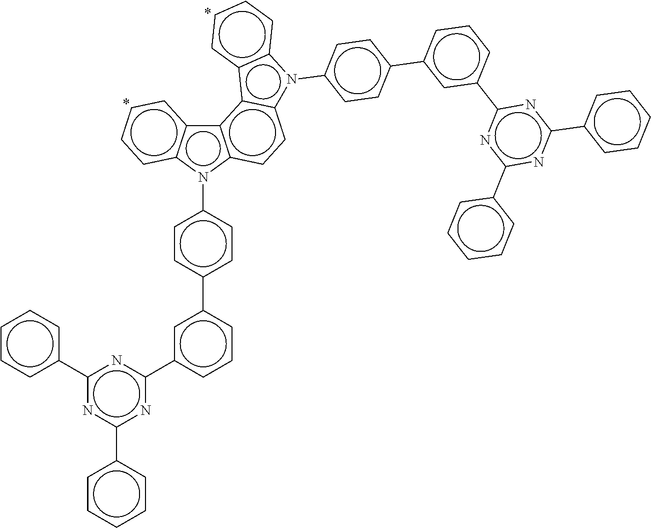 | R7 |
| 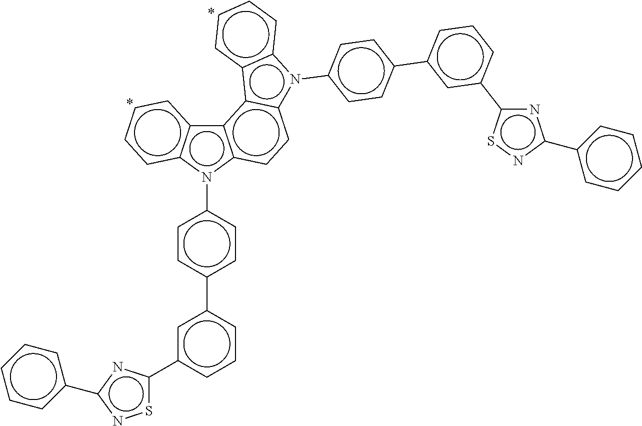 | R8 |
| 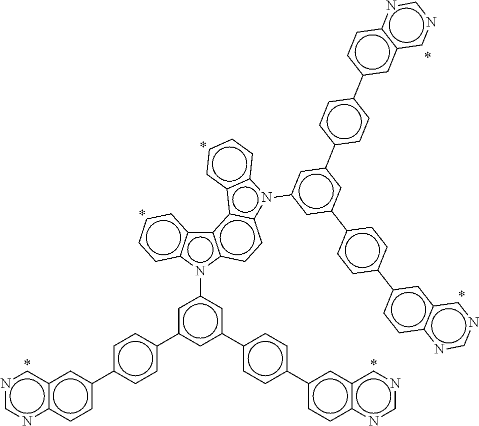 | R9 |
| 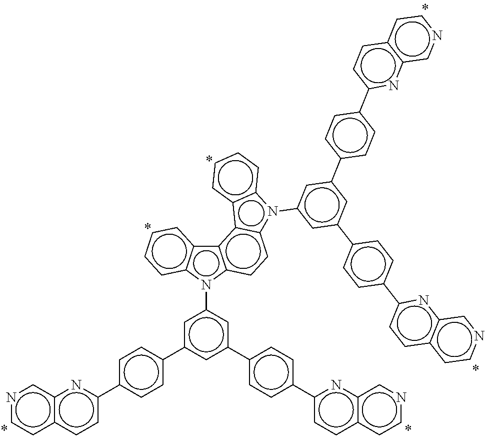 | R10 |
| 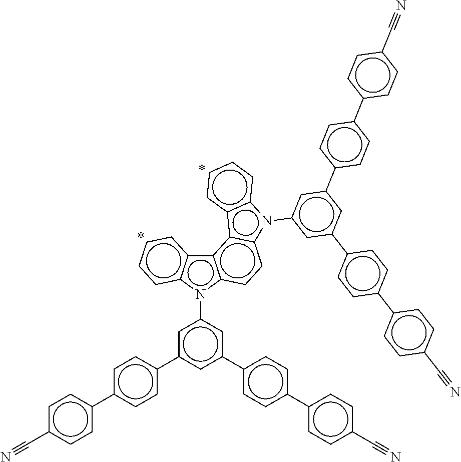 | R11 |
| 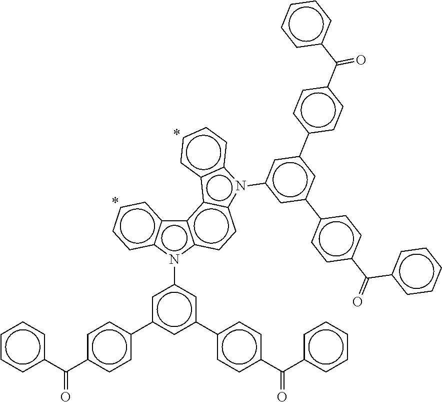 | R12 |
| 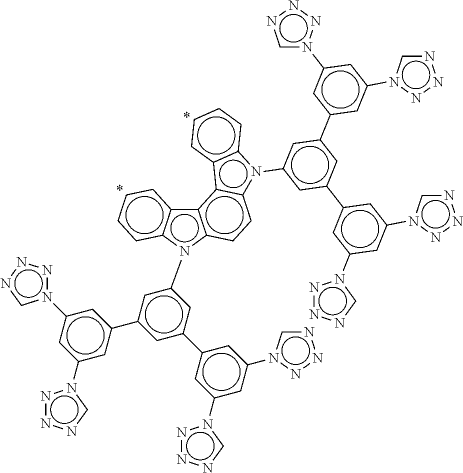 | R13 |
| 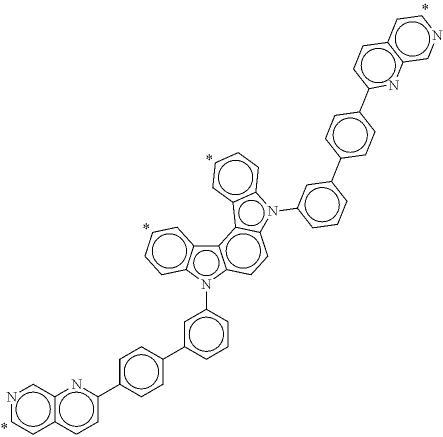 | R14 |
| 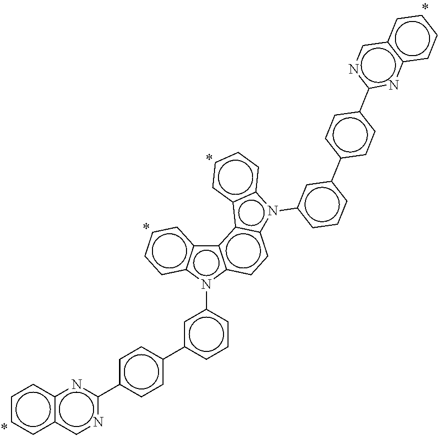 | R15 |
| 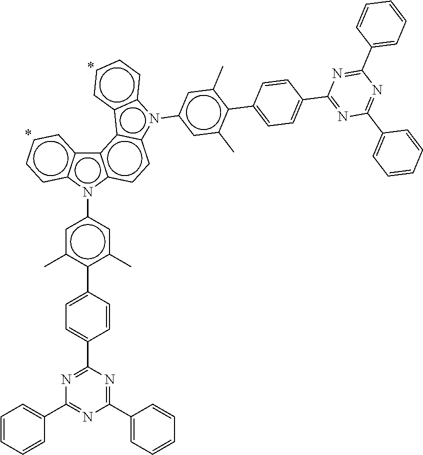 | R16 |
| 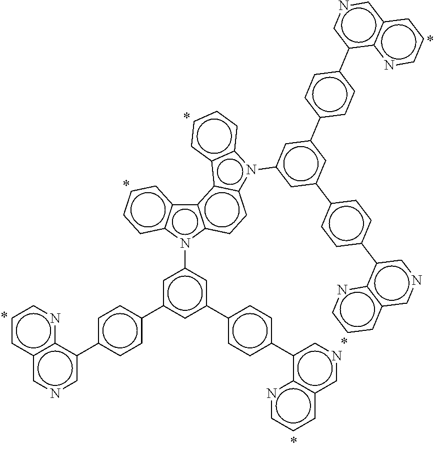 | R17 |
| 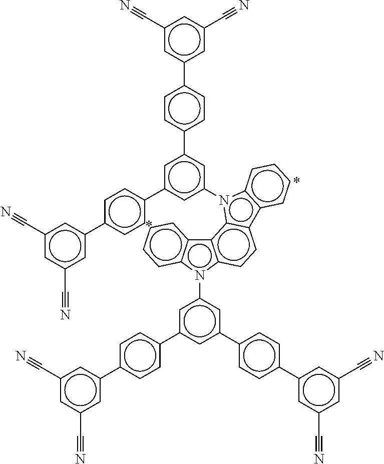 | R18 |
| 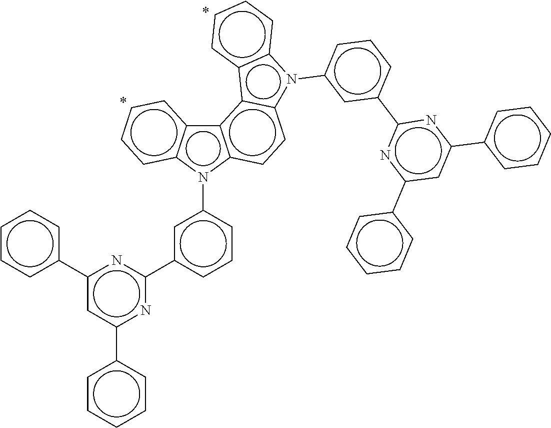 | R19 |
| 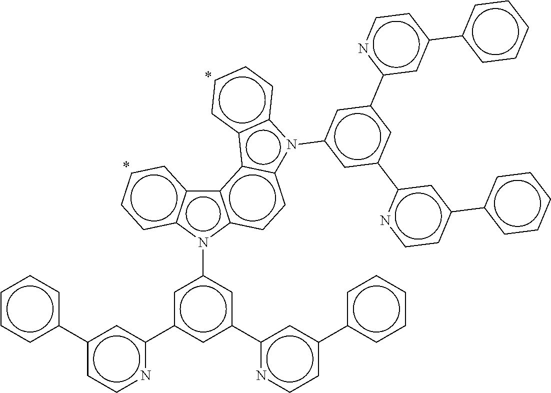 | R20 |
| 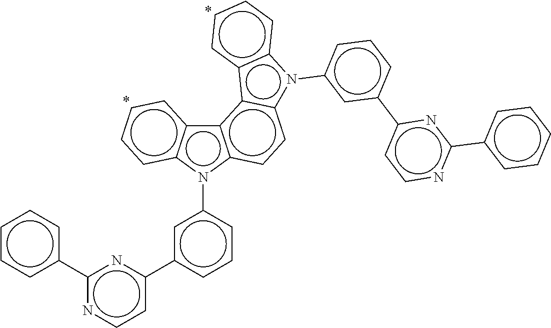 | R21 |
| 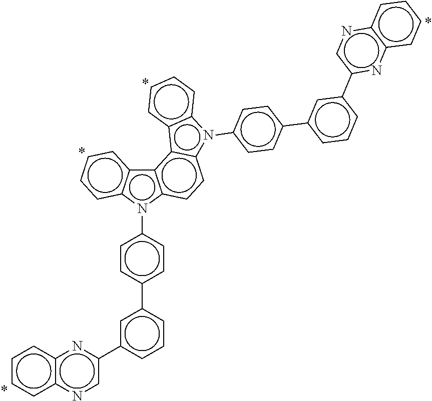 | R22 |
| 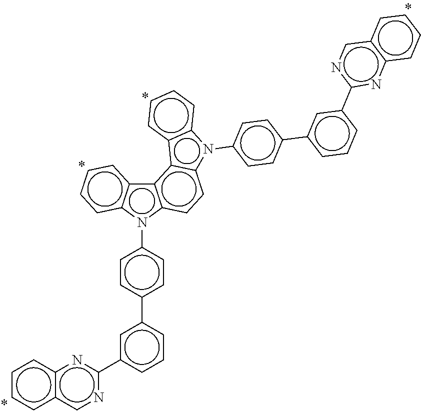 | R23 |
| 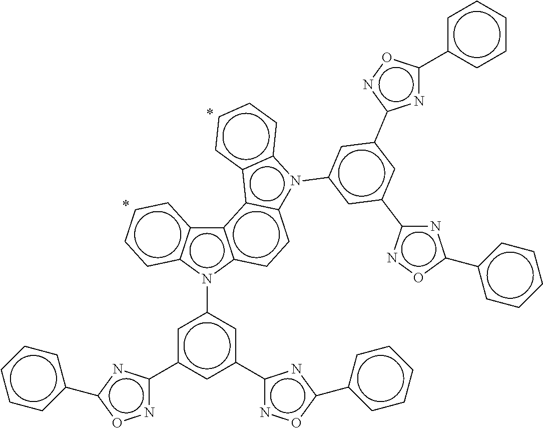 | R24 |
| 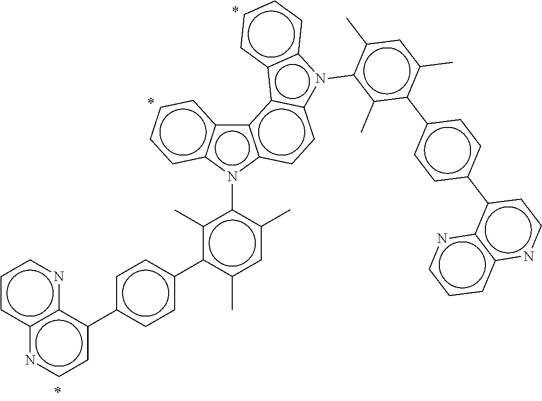 | R25 |
| 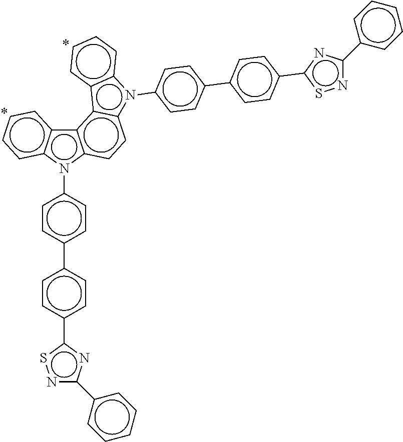 | R26 |
| 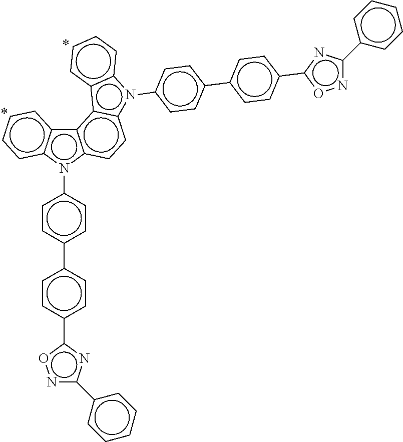 | R27 |
| 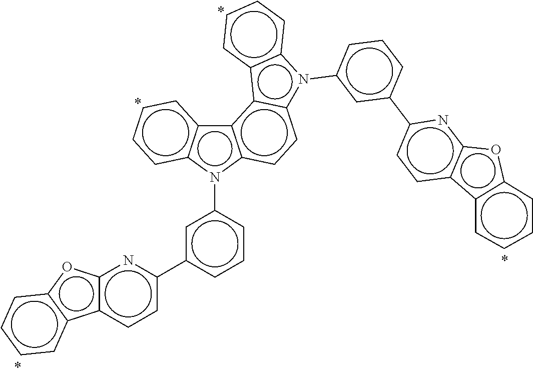 | R28 |
| 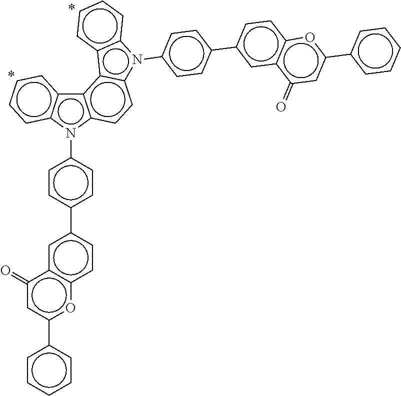 | R29 |
| 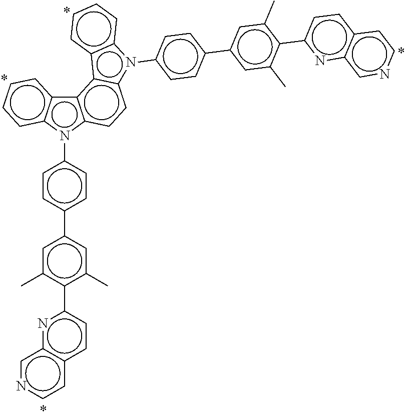 | R30 |
| 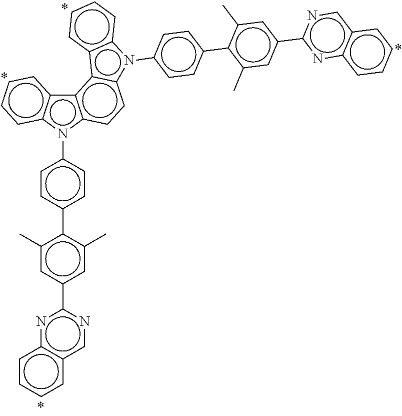 | R31 |
| 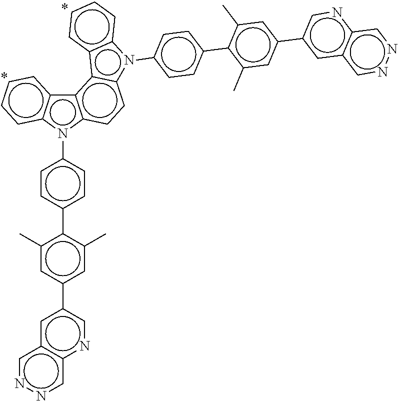 | R32 |
| 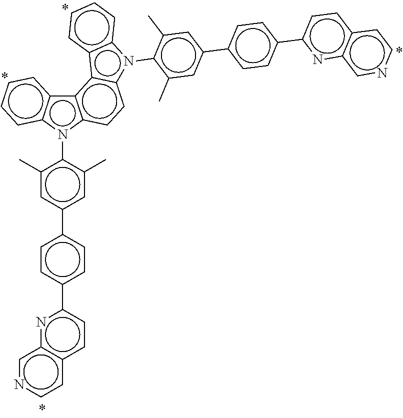 | R33 |
| 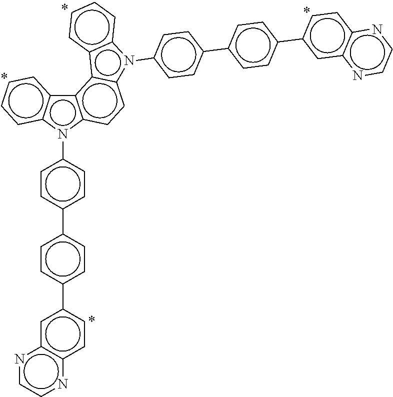 | R34 |
| 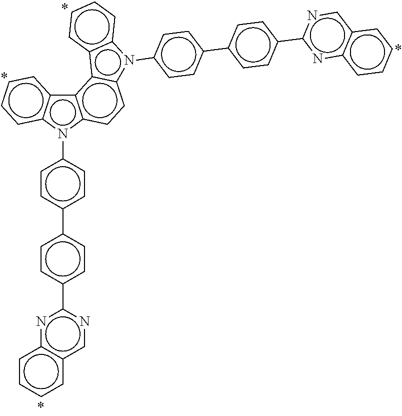 | R35 |
| 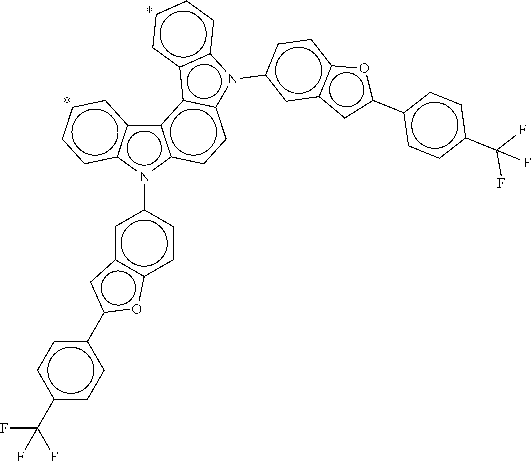 | R36 |
| 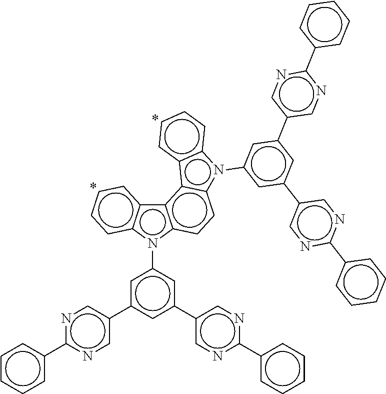 | R37 |
| 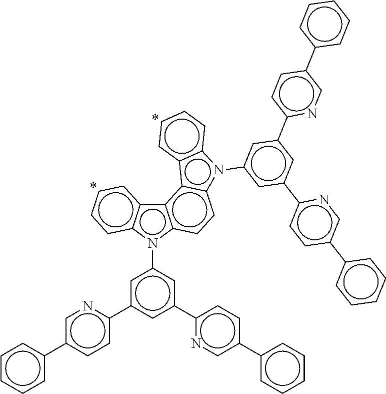 | R38 |
| 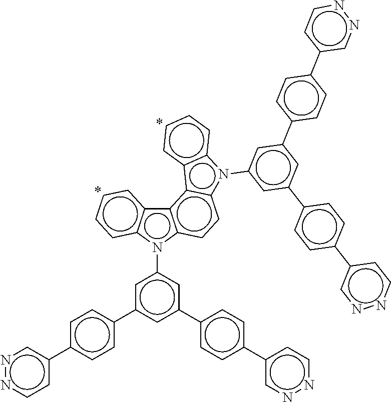 | R39 |
| 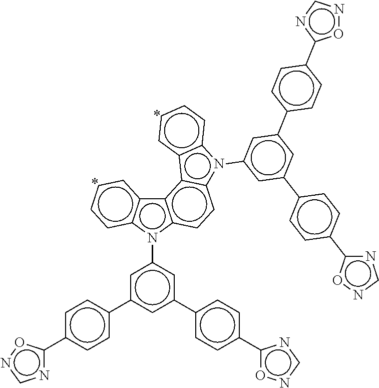 | R40 |
| 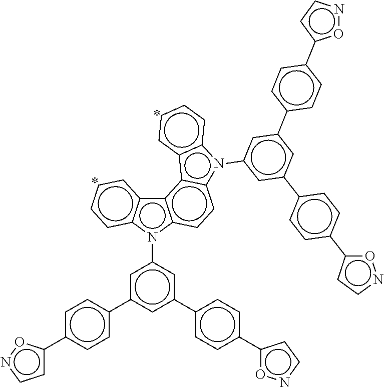 | R41 |
| 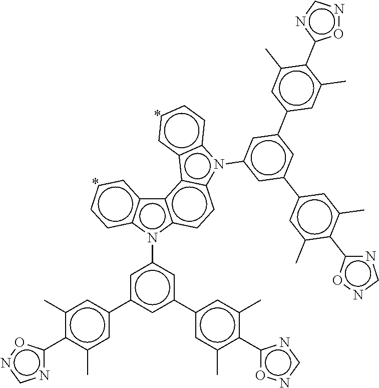 | R42 |
| 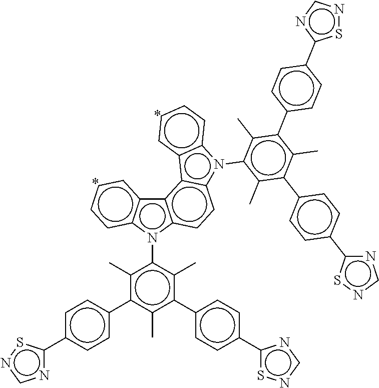 | R43 |
| 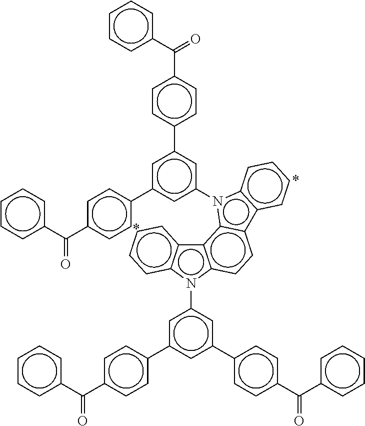 | R44 |
| 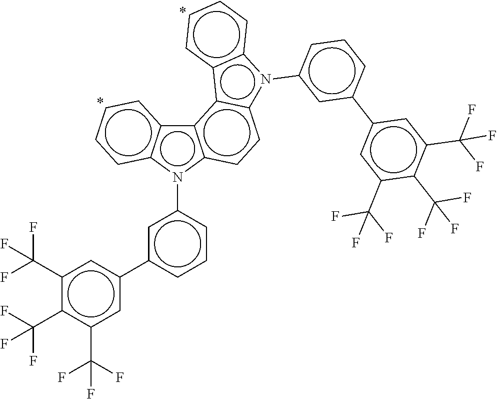 | R45 |
| 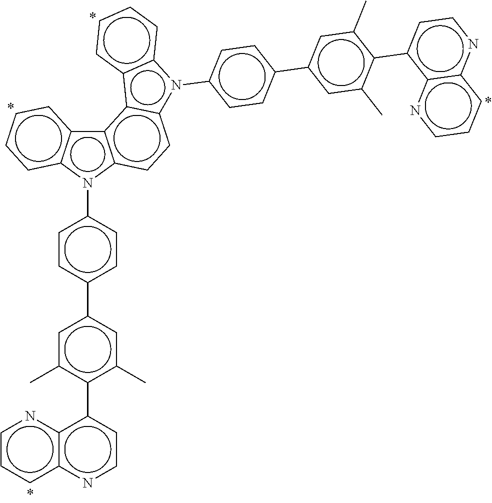 | R46 |
| 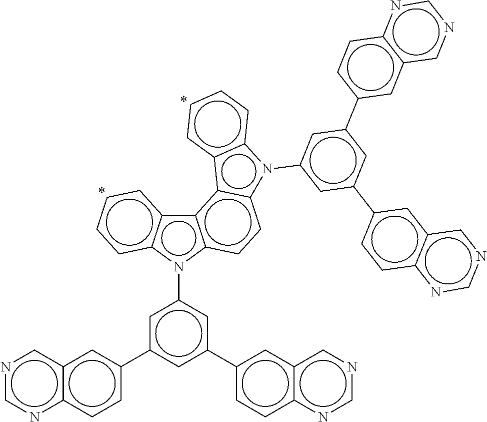 | R47 |
| 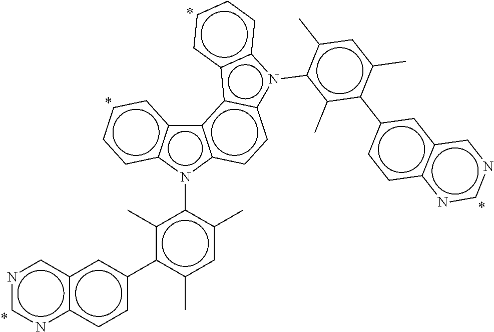 | R48 |
| 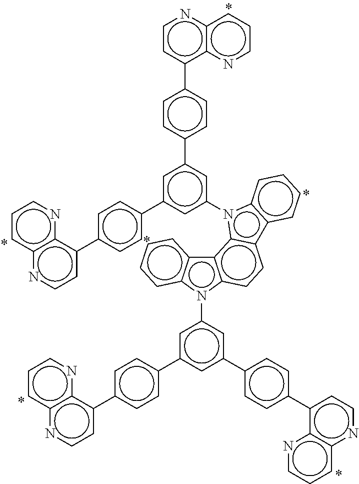 | R49 |
| 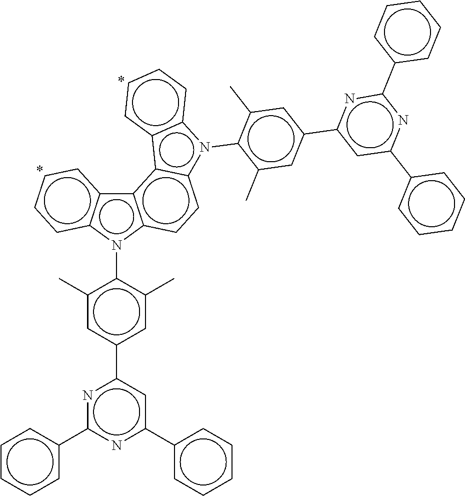 | R50 |
| 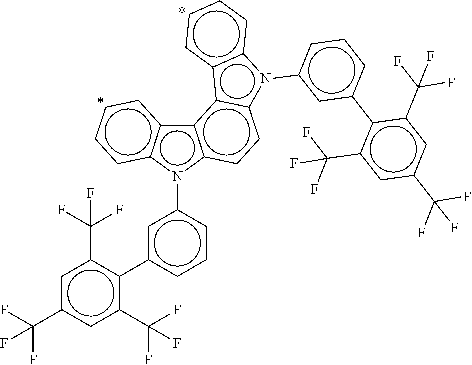 | R51 |
| 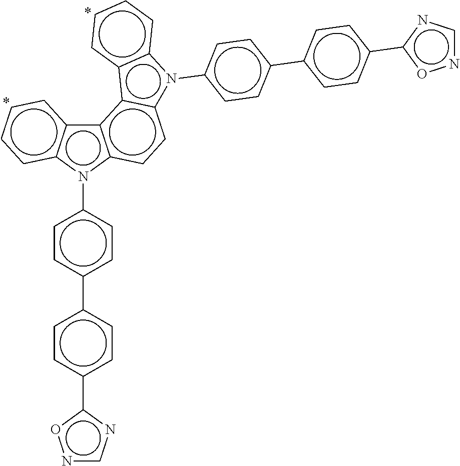 | R52 |
| 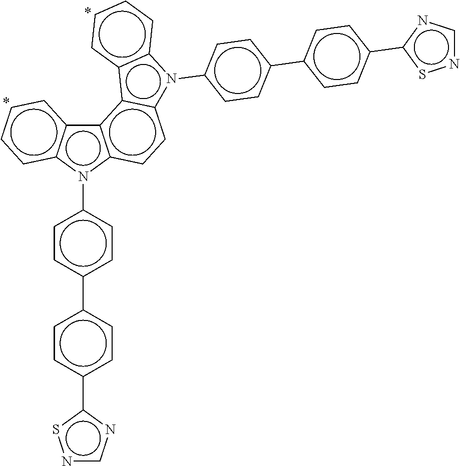 | R53 |
| 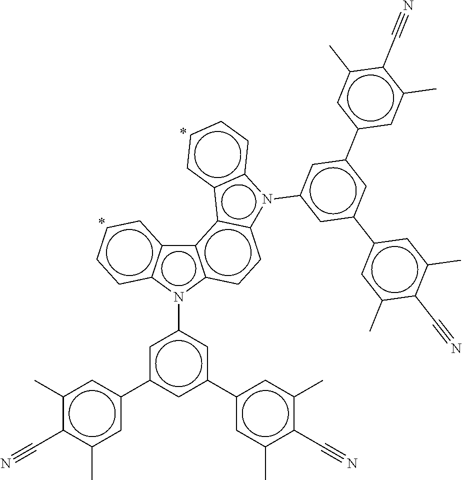 | R54 |
| 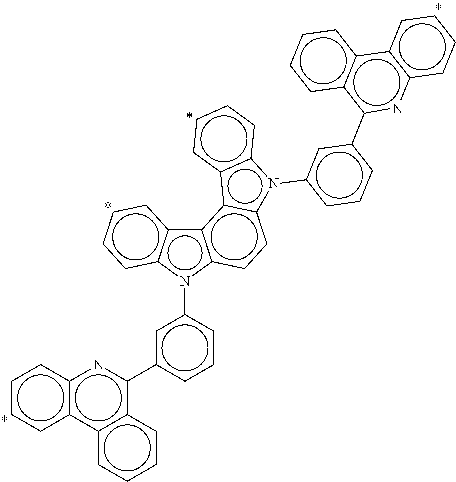 | R55 |
| 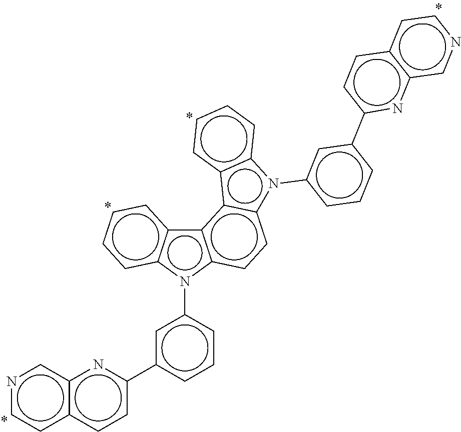 | R56 |
| 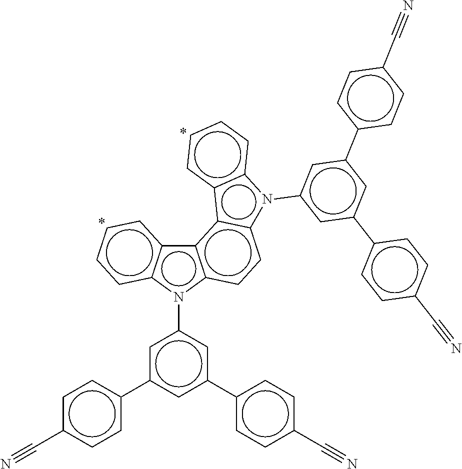 | R57 |
| 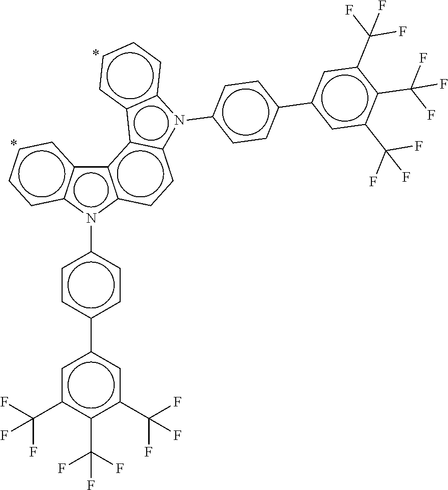 | R58 |
| 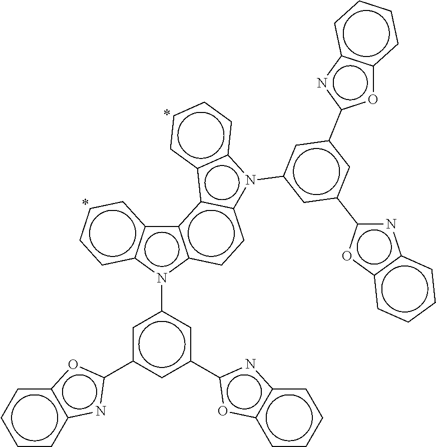 | R59 |
| 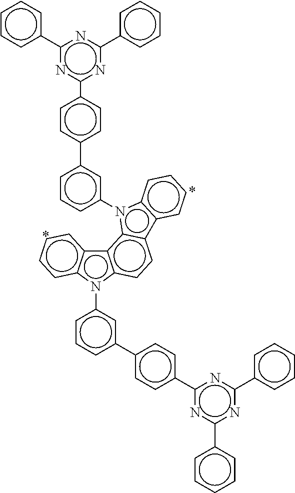 | R60 |
| 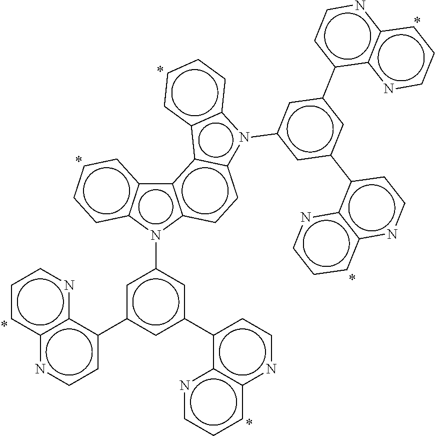 | R61 |
| 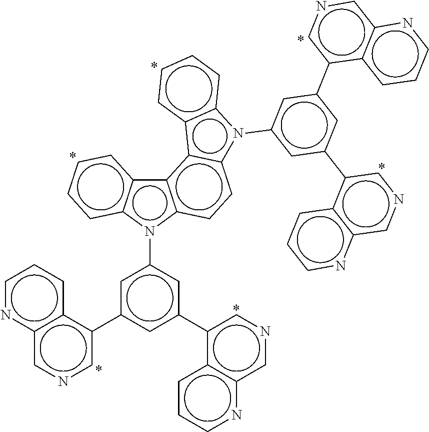 | R62 |
| 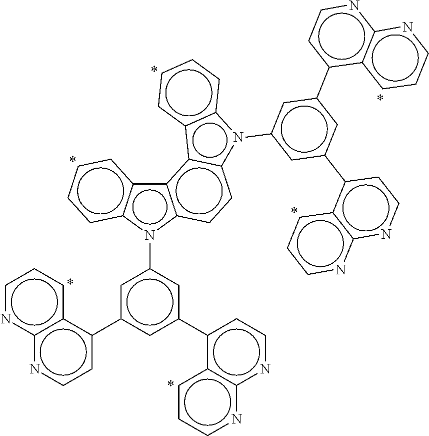 | R63 |
| 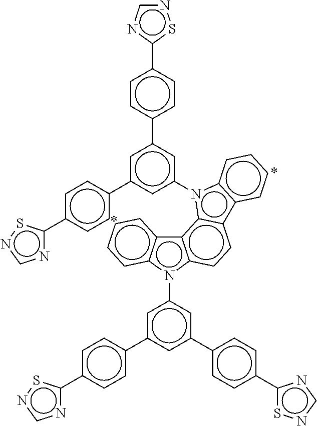 | R64 |
| 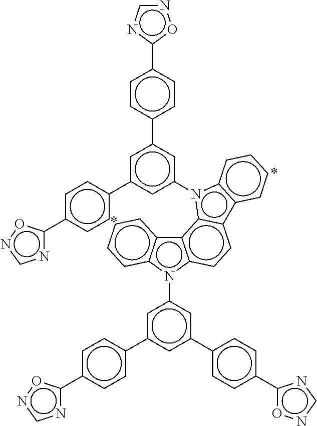 | R65 |
| 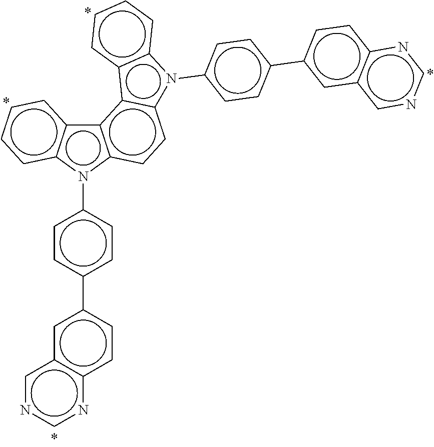 | R66 |
| 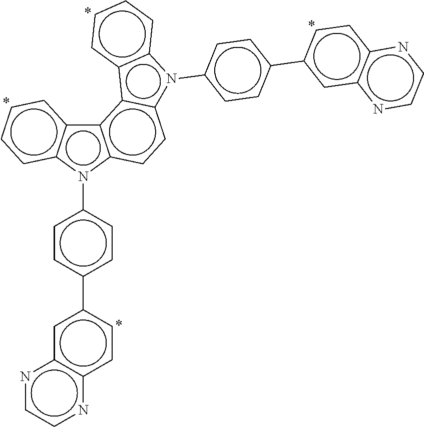 | R67 |
| 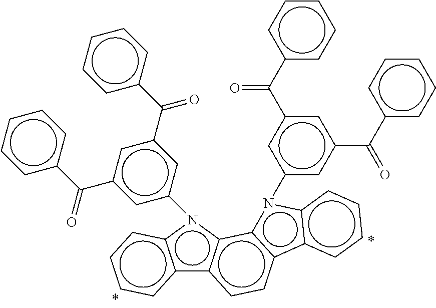 | R68 |
| 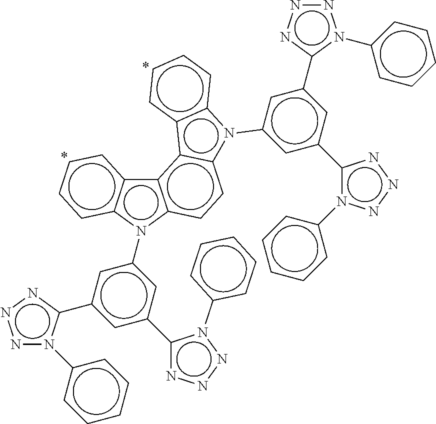 | R69 |
| 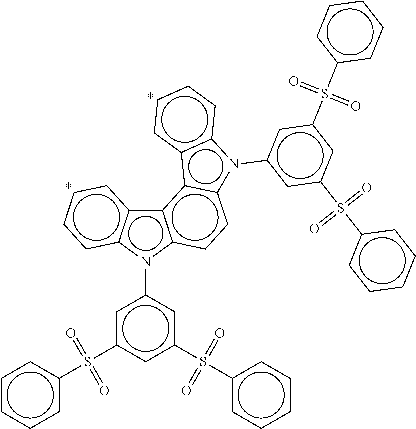 | R70 |
| 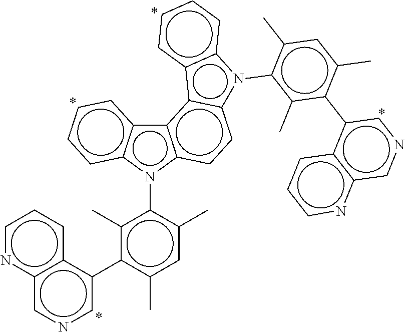 | R71 |
| 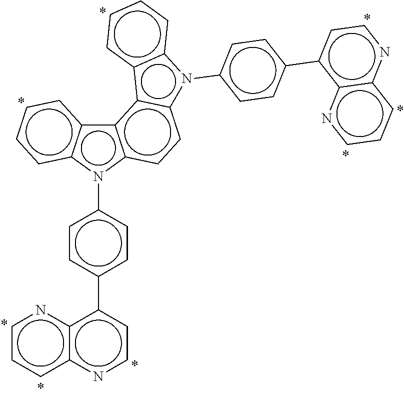 | R72 |
| 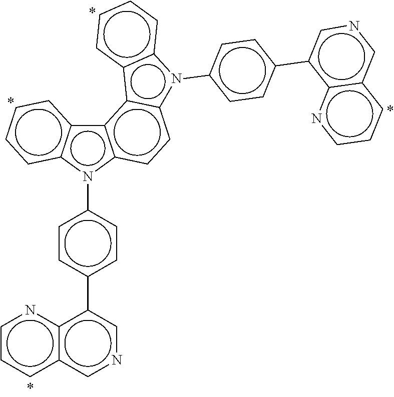 | R73 |
| 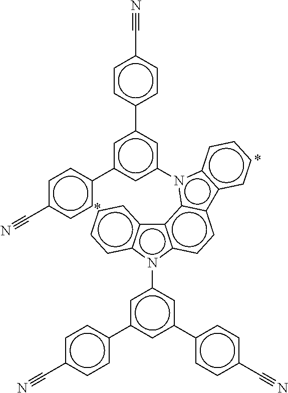 | R74 |
| 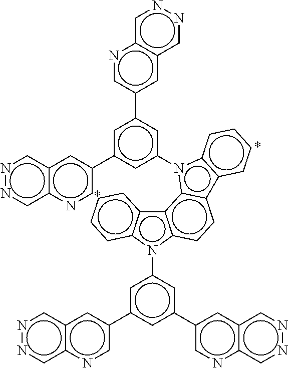 | R75 |
| 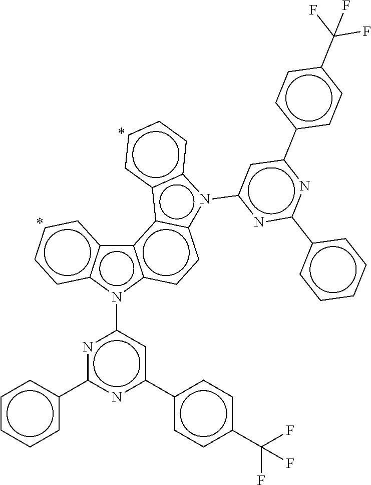 | R76 |
| 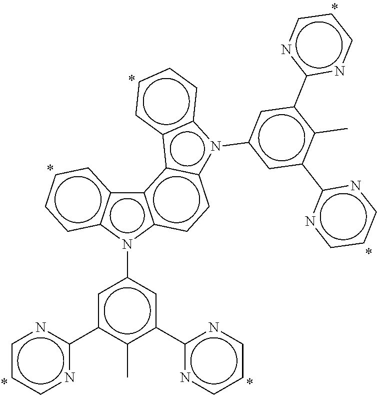 | R77 |
| 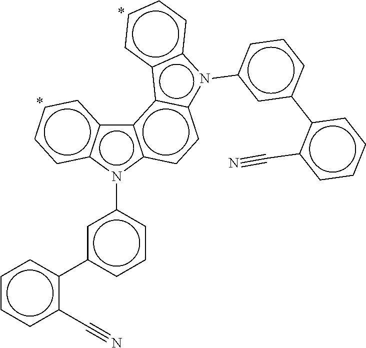 | R78 |
| 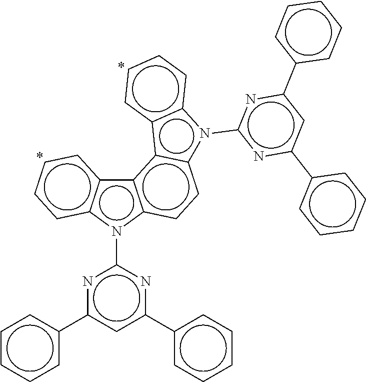 | R79 |
| 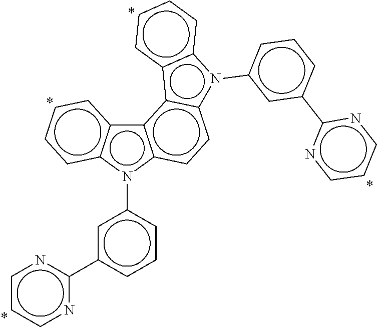 | R80 |
| 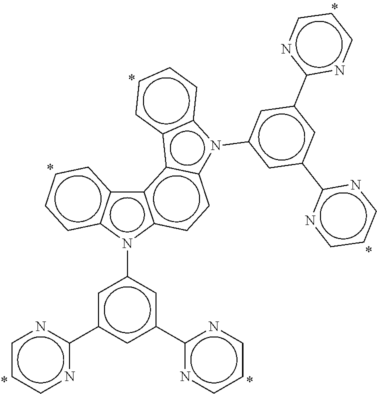 | R81 |
| 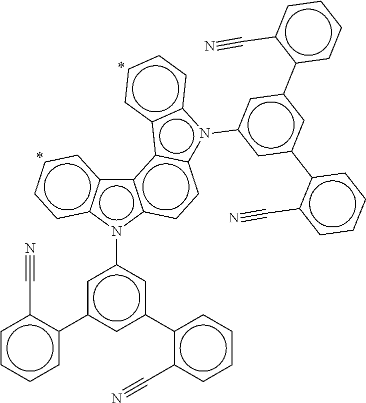 | R82 |
| 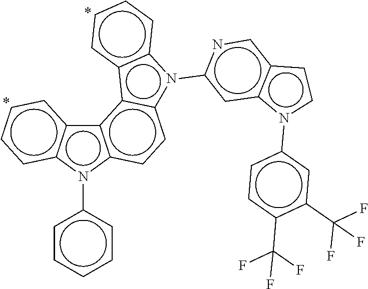 | R83 |
| 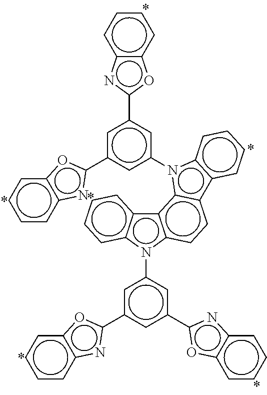 | R84 |
| 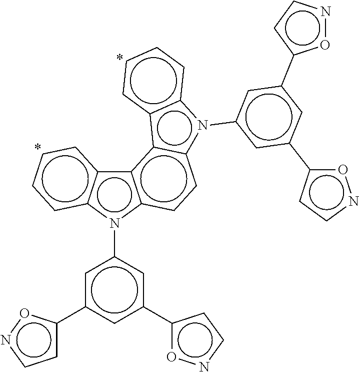 | R85 |
| 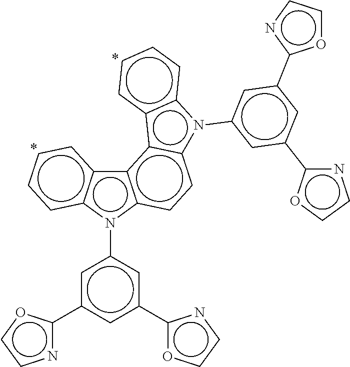 | R86 |
| 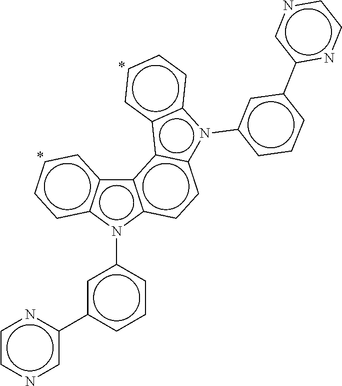 | R87 |
| 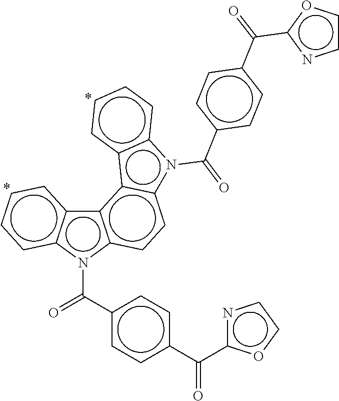 | R88 |
| 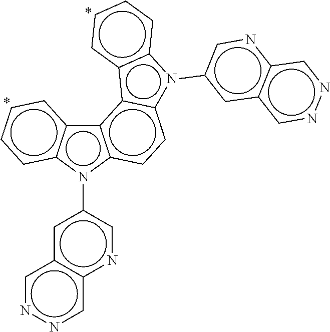 | R89 |
| 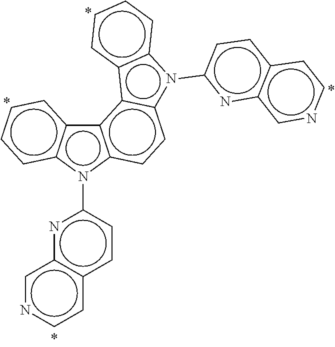 | R90 |
| 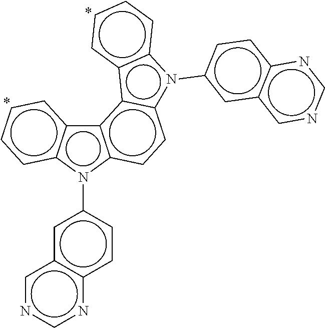 | R91 |
| 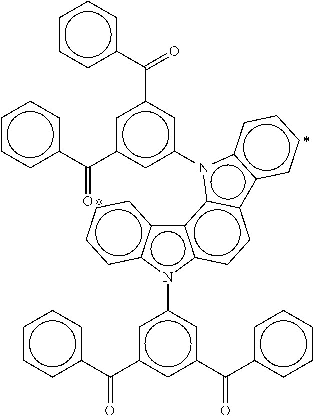 | R92 |
| 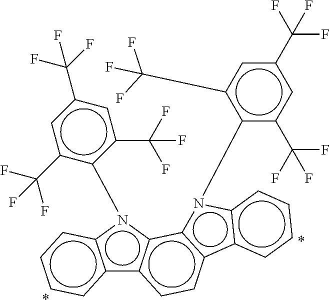 | R93 |
| 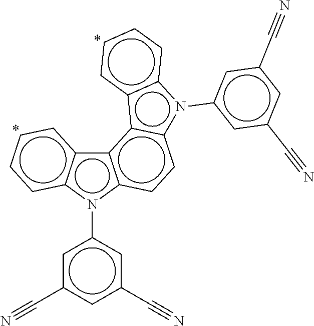 | R94 |
| 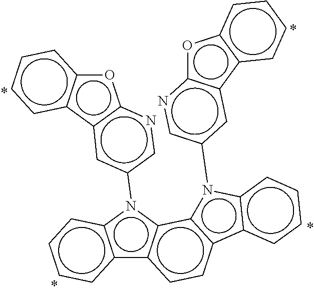 | R95 |
| 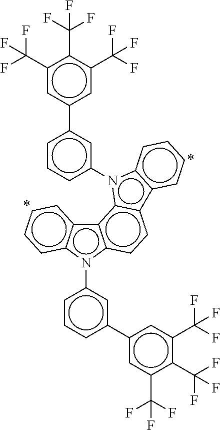 | R96 |
| 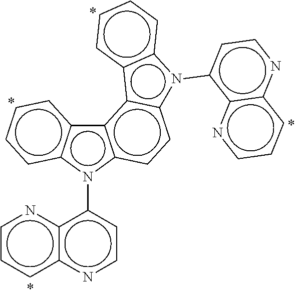 | R97 |
| 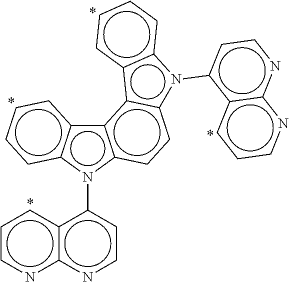 | R98 |
| 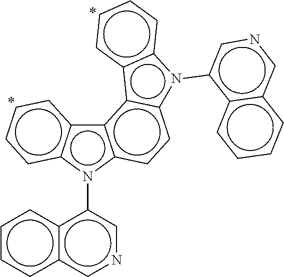 | R99 |
| 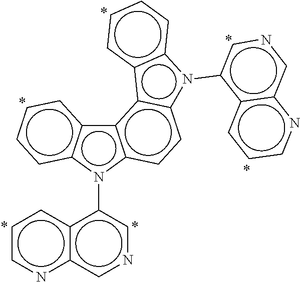 | R100 |
| 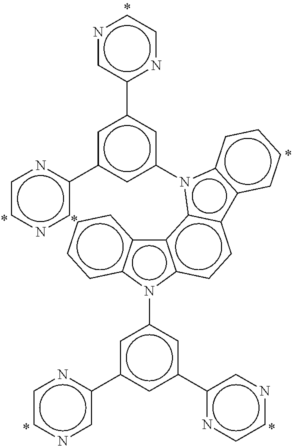 | R101 |
| 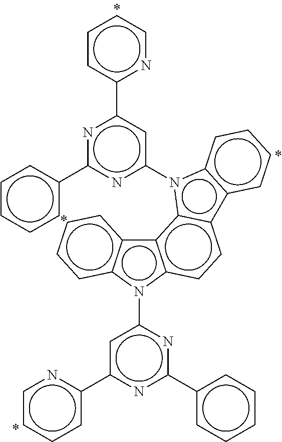 | R102 |
| 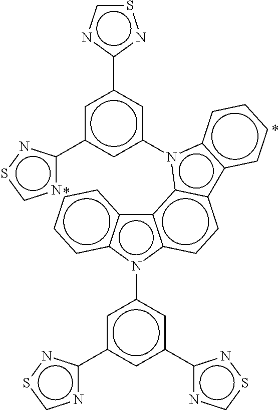 | R103 |
| 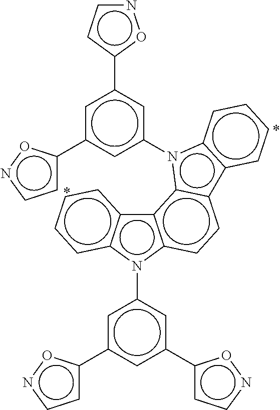 | R104 |
| 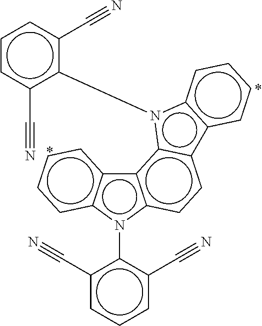 | R105 |
| 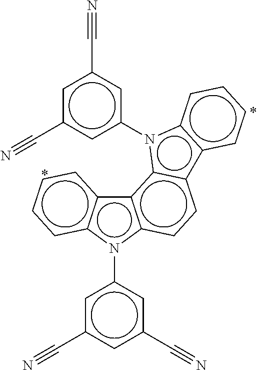 | R106 |
| 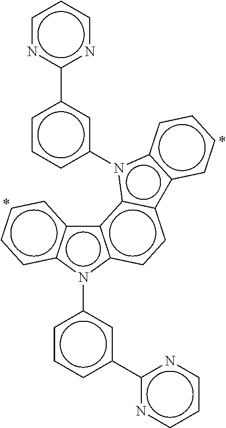 | R107 |
| 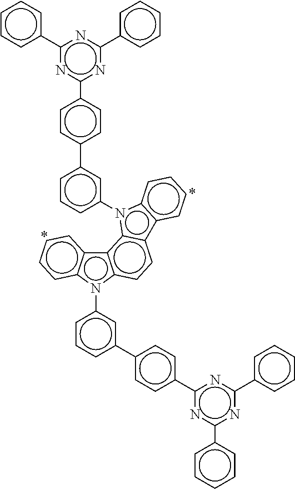 | R108 |
| 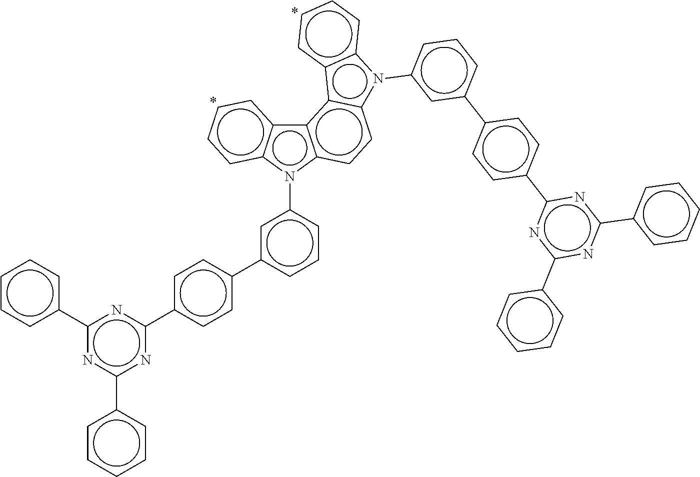 | R109 |
| 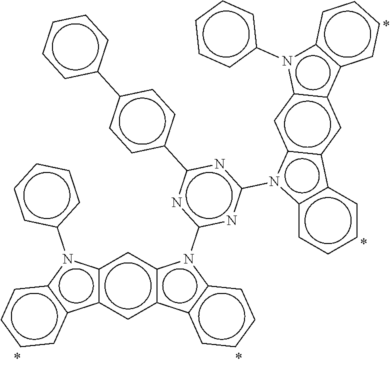 | R110 |

In example embodiments of the fourteenth aspect, the invention is any one compound selected from Table R′. As described above with respect to the fourteenth aspect, in some embodiments, any substitutable carbon in the molecule is optionally substituted by R^{C}. In some embodiments, at least one substitutable carbon in the molecule is substituted by R^{C}. In some embodiments, atoms indicated by * are optionally substituted by R^{C}. In some embodiments, at least one atom indicated by * is substituted by R^{C}. In some embodiments, the molecule is not substituted, and is represented by the structural formula as depicted. The variables may be selected as described above with respect to the fourteenth aspect.

TABLE R′

| | |
|---|---|
| 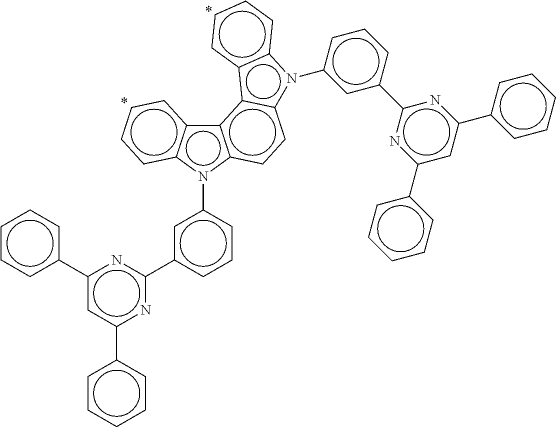 | R19 |
| 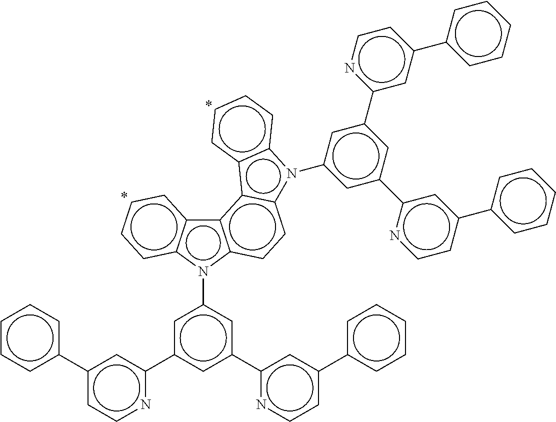 | R20 |
| 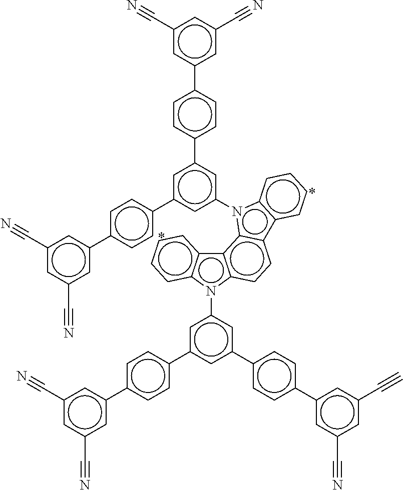 | R18 |
| 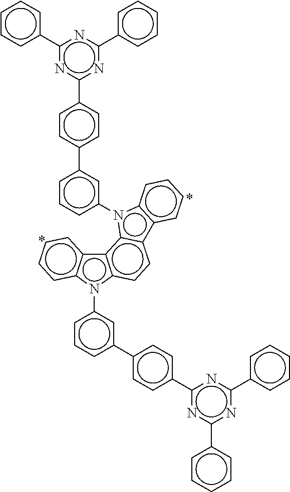 | R108 |
| 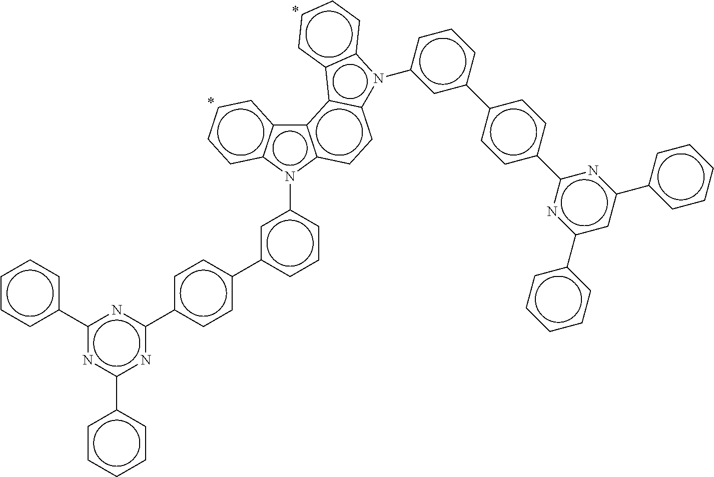 | R109 |
| 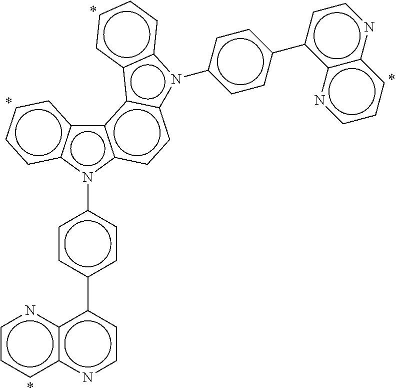 | R72 |
| 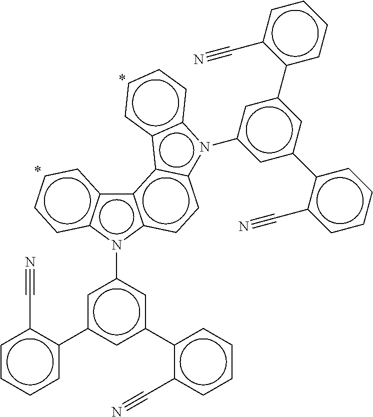 | R82 |
| 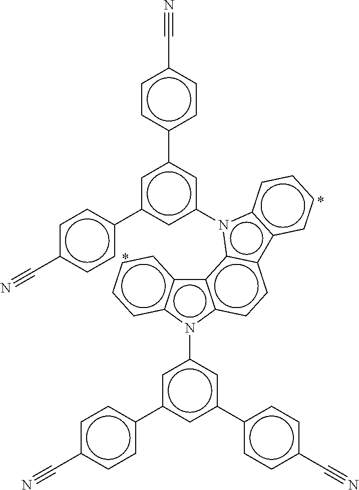 | R74 |
| 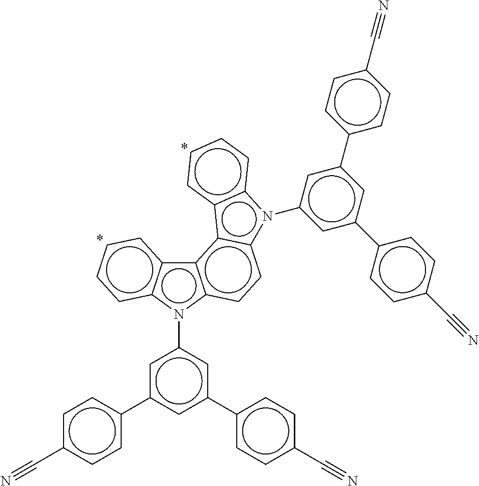 | R57 |
| 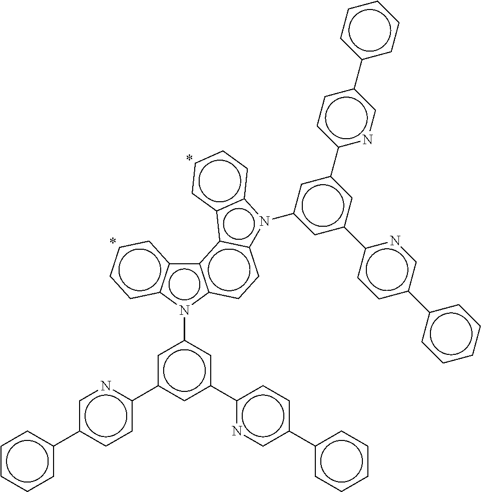 | R38 |
| 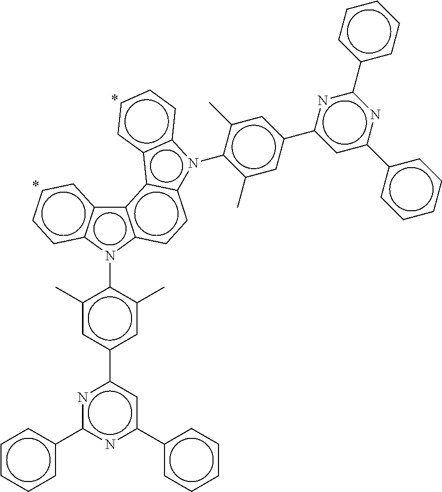 | R50 |
| 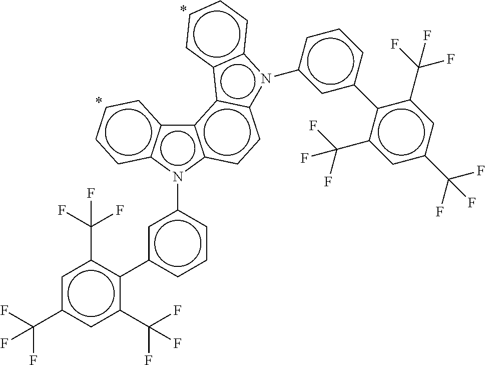 | R51 |
| 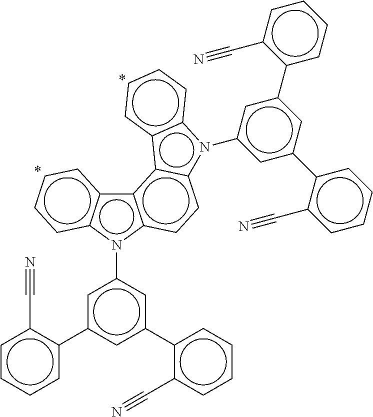 | R82 |
| 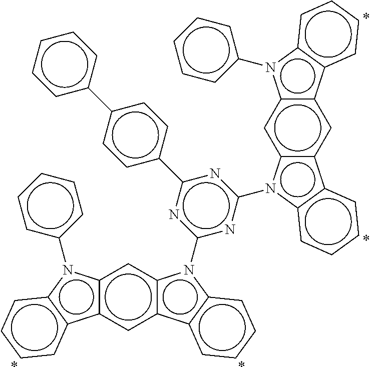 | R110 |
| 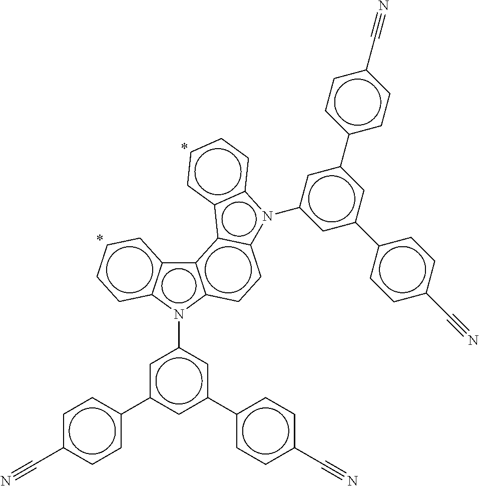 | R57 |

In a fifteenth aspect, the present invention is a molecule represented by structural formula (I):

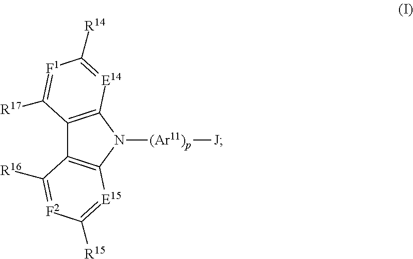

wherein:

E¹⁴, and E¹⁵, are, each independently, CR^{A} or N, wherein R^{A}, for each occurrence independently, is H, a C₁-C₆ alkyl, a C₃-C₁₈ cycloalkyl, a C₆-C₁₈ aryl, a 5-20 atom heteroaryl, a halo, or —CN;

J is any moiety selected from —CN,

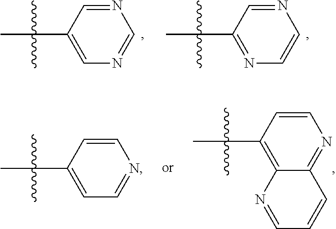 [SMILES: *c1cncnc1 ; *c1cnccn1 ; *c1ccncc1 ; "or" ; *c1ccnc2cccnc12]

and is optionally substituted with one or more R¹¹, each independently selected from C₁-C₆ alkyl, a C₃-C₁₈ cycloalkyl, a C₆-C₁₈ aryl, a 5-20 atom heteroaryl, a halo, or —CN;

R¹⁴, R¹⁵, R¹⁶, and R^{17 are}, each independently, H, a C₁-C₆ alkyl, a C₃-C₁₈ cycloalkyl, a C₆-C₁₈ aryl, a 5-20 atom heteroaryl, a halo, or —CN;

F¹ is C—(Ar¹²)_{q}-G;

F² is CR^{B} or N, wherein R^{B} is H, a C₁-C₆ alkyl, a C₃-C₆ cycloalkyl, a C₆-C₁₈aryl, a 5-20 atom heteroaryl, a halo, or —CN, or —(Ar¹²)_{q}-G;

Ar¹¹, for each occurrence independently, is phenyl optionally substituted with one to four C₁-C₆ alkyls;

Ar¹², for each occurrence independently, is phenyl optionally substituted with one to four C₁-C₆ alkyls;

p is 0, 1, or 2;

q is 0 or 1; and

G, for each occurrence independently, is a moiety represented by one of the following structural formulas:

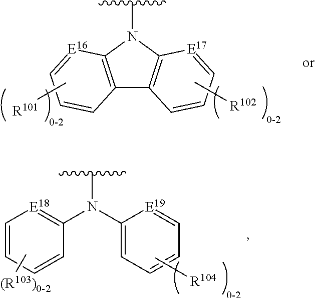

wherein:

E¹⁶, E¹⁷, E¹⁸, and E¹⁹ are, each independently, CR^{C} or N, wherein R^{C} is H, a C₁-C₃ alkyl, halo, or —CN; and

R101, R¹⁰², R¹⁰³, and R¹⁰⁴ are, each independently, a C₁-C₆ alkyl, a C₃-C₁₈ cycloalkyl, a C₆-C₁₈ aryl, a 5-20 atom heteroaryl, a halo, or —CN.

In certain embodiments of the fifteenth aspect, the molecule is not represented by any molecule represented by a structural formula in Table NN1. In certain embodiments of the fifteenth aspect, the molecule is not represented by any molecule represented by a structural formula in Table NN1, wherein the carbon or heteroatom denoted by (*) is unsubstituted or substituted by a C₁-C₆ alkyl, —OH, —CN, a halo, a C₆-C₁2 aryl, a 5-20 atom heteroaryl, —N(R¹⁹)₂, or —N(R²⁰)₂, wherein each R¹⁹, independently, is H or a C₁-C₆ alkyl, or a C₅-C₁₂ cycloalkyl, and wherein each R²⁰, independently, is H or a C₆-C₁₈ aryl.

In certain embodiments of the fifteenth aspect, E¹⁴, and E¹⁵ are, each independently, CR^{A} or N. In certain embodiments, E¹⁴ is CR^{A}. In certain embodiments, E¹⁴ is N. In certain embodiments, E¹⁵ is CR^{A}. In certain embodiments, E¹⁵ is N. The remainder of the variables in structural formula (I) are as described above and below with respect to the fifteenth aspect.

In certain embodiments of the fifteenth aspect, J is any moiety selected from —CN,

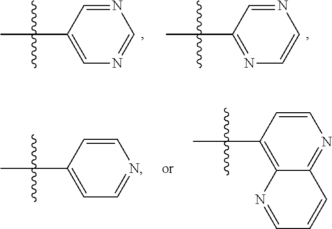 [SMILES: *c1cncnc1 ; *c1cnccn1 ; *c1ccncc1 ; "or" ; *c1ccnc2cccnc12]

and is optionally substituted with one or more R¹¹. In certain embodiments, J is unsubstituted. In certain embodiments, J is —CN. In certain embodiments, J is

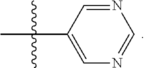 [SMILES: *c1cncnc1]

In certain embodiments, J is

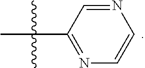 [SMILES: *c1cnccn1]

In certain embodiments, J is

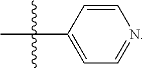 [SMILES: *c1ccncc1]

In certain embodiments, J is

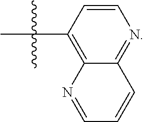 [SMILES: *c1ccnc2cccnc12]

The remainder of the variables in structural formula (I) are as described above and below with respect to the fifteenth aspect.

In certain embodiments of the fifteenth aspect, each R¹¹ is independently selected from C₁-C₆ alkyl, a C₃-C₁₈ cycloalkyl, a C₆-C₁₈ aryl, a 5-20 atom heteroaryl, a halo, or —CN. In certain embodiments, each R¹¹ is independently selected from C₁-C₆ alkyl or C₆-C₁₈ aryl. In certain embodiments, each R¹¹ is independently selected from C₁-C₆ alkyl or phenyl. In certain embodiments, each R¹¹ is independently selected from C₁-C₃ alkyl or phenyl. In certain embodiments, each R¹¹ is independently selected from methyl or phenyl. The remainder of the variables in structural formula (I) are as described above and below with respect to the fifteenth aspect.

In certain embodiments of the fifteenth aspect, R¹⁴ is H, a C₁-C₆ alkyl, a C₃-C₁₈ cycloalkyl, a C₆-C₁₈ aryl, a 5-20 atom heteroaryl, a halo, or —CN. In certain embodiments, R¹⁴ is H, a C₁-C₆ alkyl, a C₃-C₁₈ cycloalkyl, a halo, or —CN. In certain embodiments, R¹⁴ is H, a C₁-C₆ alkyl, or a C₃-C₆ cycloalkyl. In certain embodiments, R¹⁴ is H or methyl. In certain embodiments, R¹⁴ is H. In certain embodiments, R¹⁴ is a C₁-C₆ alkyl, a C₃-C₁₈ cycloalkyl, a C₆-C₁₈ aryl, a 5-20 atom heteroaryl, a halo, or —CN. In certain embodiments, R¹⁴ is a C₁-C₆ alkyl, a C₃-C₁₈ cycloalkyl, a halo, or —CN. In certain embodiments, R¹⁴ is a C₁-C₆ alkyl, or a C₃-C₆ cycloalkyl. In certain embodiments, R¹⁴ is methyl. The remainder of the variables in structural formula (I) are as described above and below with respect to the fifteenth aspect.

In certain embodiments of the fifteenth aspect, R¹⁵ is H, a C₁-C₆ alkyl, a C₃-C₁₈ cycloalkyl, a C₆-C₁₈ aryl, a 5-20 atom heteroaryl, a halo, or —CN. In certain embodiments, R¹⁵ is H, a C₁-C₆ alkyl, a C₃-C₁₈ cycloalkyl, a halo, or —CN. In certain embodiments, R¹⁵ is H, a C₁-C₆ alkyl, or a C₃-C₆ cycloalkyl. In certain embodiments, R¹⁵ is H or methyl. In certain embodiments, R¹⁵ is H. In certain embodiments, R¹⁵ is a C₁-C₆ alkyl, a C₃-C₁₈ cycloalkyl, a C₆-C₁₈ aryl, a 5-20 atom heteroaryl, a halo, or —CN. In certain embodiments, R¹⁵ is a C₁-C₆ alkyl, a C₃-C₁₈ cycloalkyl, a halo, or —CN. In certain embodiments, R¹⁵ is a C₁-C₆ alkyl, or a C₃-C₆ cycloalkyl. In certain embodiments, R¹⁵ is methyl. The remainder of the variables in structural formula (I) are as described above and below with respect to the fifteenth aspect.

In certain embodiments of the fifteenth aspect, R¹⁶ is H, a C₁-C₆ alkyl, a C₃-C₁₈ cycloalkyl, a C₆-C₁₈ aryl, a 5-20 atom heteroaryl, a halo, or —CN. In certain embodiments, R¹⁶ is H, a C₁-C₆ alkyl, a C₃-C₁₈ cycloalkyl, a halo, or —CN. In certain embodiments, R¹⁶ is H, a C₁-C₆ alkyl, or a C₃-C₆ cycloalkyl. In certain embodiments, R¹⁶ is H or methyl. In certain embodiments, R¹⁶ is H. In certain embodiments, R¹⁶ is a C₁-C₆ alkyl, a C₃-C₁₈ cycloalkyl, a C₆-C₁₈ aryl, a 5-20 atom heteroaryl, a halo, or —CN. In certain embodiments, R¹⁶ is a C₁-C₆ alkyl, a C₃-C₁₈ cycloalkyl, a halo, or —CN. In certain embodiments, R¹⁶ is a C₁-C₆ alkyl, or a C₃-C₆ cycloalkyl. In certain embodiments, R¹⁶ is methyl. The remainder of the variables in structural formula (I) are as described above and below with respect to the fifteenth aspect.

In certain embodiments of the fifteenth aspect, R¹⁷ is H, a C₁-C₆ alkyl, a C₃-C₁₈ cycloalkyl, a C₆-C₁₈ aryl, a 5-20 atom heteroaryl, a halo, or —CN. In certain embodiments, R¹⁷ is H, a C₁-C₆ alkyl, a C₃-C₁₈ cycloalkyl, a halo, or —CN. In certain embodiments, R¹⁷ is H, a C₁-C₆ alkyl, or a C₃-C₆ cycloalkyl. In certain embodiments, R¹⁶ is H or methyl. In certain embodiments, R¹⁷ is H. In certain embodiments, R¹⁷ is a C₁-C₆ alkyl, a C₃-C₁₈ cycloalkyl, a C₆-C₁₈ aryl, a 5-20 atom heteroaryl, a halo, or —CN. In certain embodiments, R¹⁷ is a C₁-C₆ alkyl, a C₃-C₁₈ cycloalkyl, a halo, or —CN. In certain embodiments, R¹⁷ is a C₁-C₆ alkyl, or a C₃-C₆ cycloalkyl. In certain embodiments, R¹⁷ is methyl. The remainder of the variables in structural formula (I) are as described above and below with respect to the fifteenth aspect.

In certain embodiments of the fifteenth aspect, F¹ is C—(Ar¹²)_{q}-G. The remainder of the variables in structural formula (I) are as described above and below with respect to the fifteenth aspect.

In certain embodiments of the fifteenth aspect, F² is N or CR^{B}. In certain embodiments, F² is CR^{B}. In certain embodiments, F² is N. The remainder of the variables in structural formula (I) are as described above and below with respect to the fifteenth aspect.

In certain embodiments of the fifteenth aspect, each R^{B} is, independently, H, a C₁-C₆ alkyl, a C₃-C₆ cycloalkyl, a C₆-C₁₈aryl, a 5-20 atom heteroaryl, a halo, or —CN, or —(Ar¹²)_{q}-G. In certain embodiments, each R^{B} is, independently, H, a C₁-C₆ alkyl, a C₃-C₆ cycloalkyl, or —(Ar¹²)_{q}-G. In certain embodiments, each R^{B} is, independently, H, a C₁-C₆ alkyl, a C₃-C₆ cycloalkyl, or a moiety represented by one of the following structural formulas:

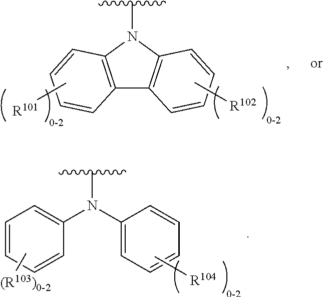

In certain embodiments, each R^{B} is, independently, H or a moiety represented by the following structural formula:

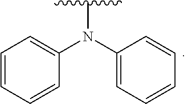 [SMILES: *N(c1ccccc1)c1ccccc1]

In certain embodiments, each R^{B} is, independently, H or a moiety represented by the following structural formula:

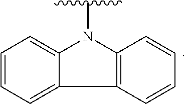 [SMILES: *n1c2ccccc2c2ccccc21]

The remainder of the variables in structural formula (I) are as described above and below with respect to the fifteenth aspect.

In certain embodiments of the fifteenth aspect, Ar¹¹, for each occurrence independently, is phenyl optionally substituted with one to four C₁-C₆ alkyls. In certain embodiments, Ar¹¹, for each occurrence independently, is phenyl optionally substituted with one to four C₁-C₃ alkyls. In certain embodiments, Ar¹¹, for each occurrence independently, is phenyl optionally substituted with one to four methyls. In certain embodiments, Ar¹¹, for each occurrence, is unsubstituted phenyl. In certain embodiments, Ar¹¹, for each occurrence independently, is phenyl optionally substituted with one to four C₂-C₆ alkyls. The remainder of the variables in structural formula (I) are as described above and below with respect to the fifteenth aspect.

In certain embodiments of the fifteenth aspect, Ar¹², for each occurrence independently, is phenyl optionally substituted with one to four C₁-C₆ alkyls. In certain embodiments, Ar¹², for each occurrence independently, is phenyl optionally substituted with one to four C₁-C₃ alkyls. In certain embodiments, Ar¹², for each occurrence independently, is phenyl optionally substituted with one to four methyls. In certain embodiments, Ar¹², for each occurrence, is unsubstituted phenyl. In certain embodiments, Ar¹², for each occurrence independently, is phenyl optionally substituted with one to four C₂-C₆ alkyls. The remainder of the variables in structural formula (I) are as described above and below with respect to the fifteenth aspect.

In certain embodiments of the fifteenth aspect, p is 0 or 1. In certain embodiments, p is 0. In certain embodiments, p is 1. The remainder of the variables in structural formula (I) are as described above and below with respect to the fifteenth aspect.

In certain embodiments of the fifteenth aspect, q is 0 or 1. In certain embodiments, q is 0. In certain embodiments, q is 1. The remainder of the variables in structural formula (I) are as described above and below with respect to the fifteenth aspect.

In certain embodiments of the fifteenth aspect, G, for each occurrence independently, is a moiety represented by one of the following structural formulas:

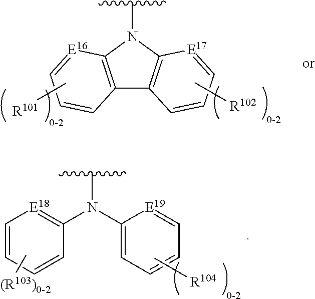

In certain embodiments, G is a moiety represented by one of the following structural formulas:

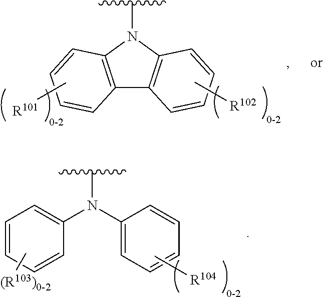

In certain embodiments, G is a moiety represented the following structural formula:

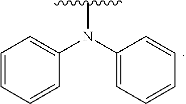 [SMILES: *N(c1ccccc1)c1ccccc1]

In certain embodiments, G is a moiety represented the following structural formula:

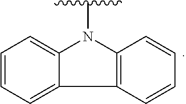 [SMILES: *n1c2ccccc2c2ccccc21]

The remainder of the variables in structural formula (I) are as described above and below with respect to the fifteenth aspect.

In certain embodiments of the fifteenth aspect, E¹⁶ is CR^{C} or N. In certain embodiments, E¹⁶ is CR^{C}. In certain embodiments, E¹⁶ is N. The remainder of the variables in structural formula (I) are as described above and below with respect to the fifteenth aspect.

In certain embodiments of the fifteenth aspect, E¹⁷ is CR^{C} or N. In certain embodiments, E¹⁷ is CR^{C}. In certain embodiments, E¹⁷ is N. The remainder of the variables in structural formula (I) are as described above and below with respect to the fifteenth aspect.

In certain embodiments of the fifteenth aspect, E¹⁸ is CR^{C} or N. In certain embodiments, E¹⁸ is CR^{C}. In certain embodiments, E¹⁸ is N. The remainder of the variables in structural formula (I) are as described above and below with respect to the fifteenth aspect.

In certain embodiments of the fifteenth aspect, E¹⁹ is CR^{C} or N. In certain embodiments, E¹⁹ is CR^{C}. In certain embodiments, E¹⁹ is N. The remainder of the variables in structural formula (I) are as described above and below with respect to the fifteenth aspect.

In certain embodiments of the fifteenth aspect, each R^{C} is, independently, H, a C₁-C₃ alkyl, halo, or —CN. In certain embodiments, each R^{C} is H. The remainder of the variables in structural formula (I) are as described above and below with respect to the fifteenth aspect.

In certain embodiments of the fifteenth aspect, each R¹⁰² is, independently, a C₁-C₆ alkyl, a C₃-C₁₈ cycloalkyl, a C₆-C₁₈ aryl, a 5-20 atom heteroaryl, a halo, or —CN. In certain embodiments of the fifteenth aspect, each R¹⁰² is, independently, a C₁-C₆ alkyl or a C₃-C₆ cycloalkyl. The remainder of the variables in structural formula (I) are as described above and below with respect to the fifteenth aspect.

In certain embodiments of the fifteenth aspect, each R¹⁰² is, independently, a C₁-C₆ alkyl, a C₃-C₁₈ cycloalkyl, a C₆-C₁₈ aryl, a 5-20 atom heteroaryl, a halo, or —CN. In certain embodiments of the fifteenth aspect, each R¹⁰² is, independently, a C₁-C₆ alkyl or a C₃-C₆ cycloalkyl. The remainder of the variables in structural formula (I) are as described above and below with respect to the fifteenth aspect.

In certain embodiments of the fifteenth aspect, each R¹⁰³ is, independently, a C₁-C₆ alkyl, a C₃-C₁₈ cycloalkyl, a C₆-C₁₈ aryl, a 5-20 atom heteroaryl, a halo, or —CN. In certain embodiments of the fifteenth aspect, each R¹⁰³ is, independently, a C₁-C₆ alkyl or a C₃-C₆ cycloalkyl. The remainder of the variables in structural formula (I) are as described above and below with respect to the fifteenth aspect.

In certain embodiments of the fifteenth aspect, each R¹⁰⁴ is, independently, a C₁-C₆ alkyl, a C₃-C₁₈ cycloalkyl, a C₆-C₁₈ aryl, a 5-20 atom heteroaryl, a halo, or —CN. In certain embodiments of the fifteenth aspect, each R¹⁰⁴ is, independently, a C₁-C₆ alkyl or a C₃-C₆ cycloalkyl. The remainder of the variables in structural formula (I) are as described above and below with respect to the fifteenth aspect.

In certain embodiments of the fifteenth aspect:

R^{A}, for each occurrence independently, is H or a C₁-C₆ alkyl;

R¹⁴, R¹⁵, R¹⁶, and R¹⁷ are, each independently, H, a C₁-C₆ alkyl, or a C₃-C₆ cycloalkyl;

F² is CR^{B}; and

R^{B} is H, a C₁-C₆ alkyl, a C₃-C₆ cycloalkyl, or —(Ar¹²)_{q}-G.

The remainder of the variables in structural formula (I) are as described above and below with respect to the fifteenth aspect.

In certain embodiments of the fifteenth aspect, the molecule is represented by the following structural formula:

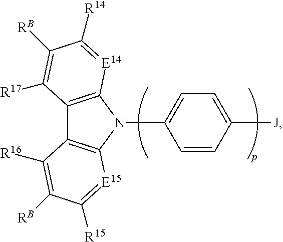

wherein the variables in structural formula (II) are as described above and below with respect to the sixteenth aspect. In certain embodiments:

E¹⁴, and E¹⁵, are, each independently, CR^{A} or N, wherein R^{A}, for each occurrence independently, is H or a C₁-C₆ alkyl;

p is 0 or 1;

R¹¹ is a C₆-C₁₈ aryl or a 5-20 atom heteroaryl;

R¹⁴, R¹⁵, R¹⁶, and R¹⁷ are, each independently, H, a C₁-C₆ alkyl, a C₃-C₁₈ cycloalkyl, a halo, or —CN;

R^{B} is, for each occurrence independently, H, a C₁-C₆ alkyl, a C₃-C₆ cycloalkyl, or a moiety represented by one of the following structural formulas:

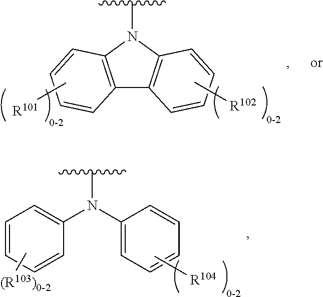

wherein:

R¹⁰¹, R¹⁰², R¹⁰³, and R¹⁰⁴ are, each independently, a C₁-C₆ alkyl or a C₃-C₆ cycloalkyl.

In certain embodiments of the fifteenth aspect, J is —CN. In certain embodiments, the molecule is represented by one of the following structural formulas:

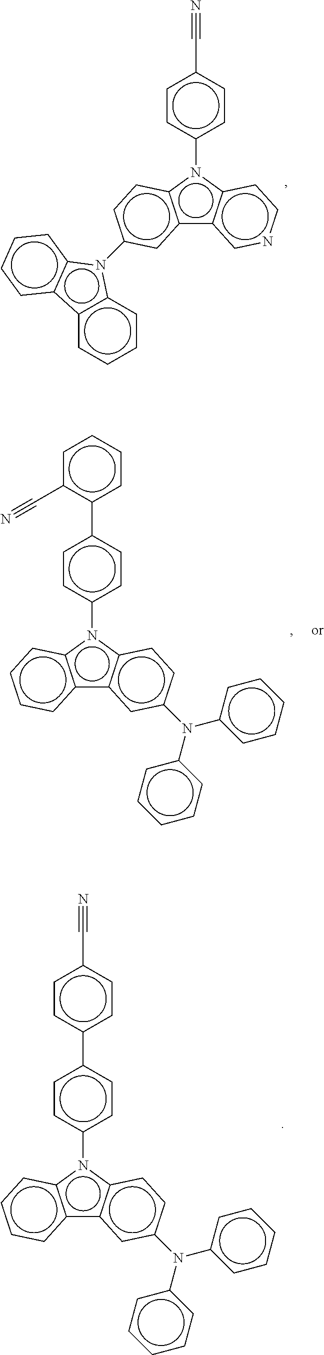

In certain embodiments of the fifteenth aspect, J is

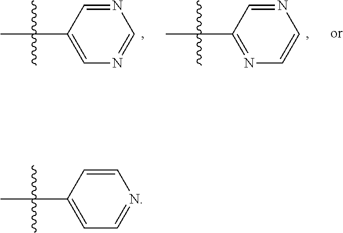 [SMILES: *c1cncnc1 ; *c1cnccn1 ; *c1ccncc1]

In certain embodiments, the molecule is represented by one of the following structural formulas:

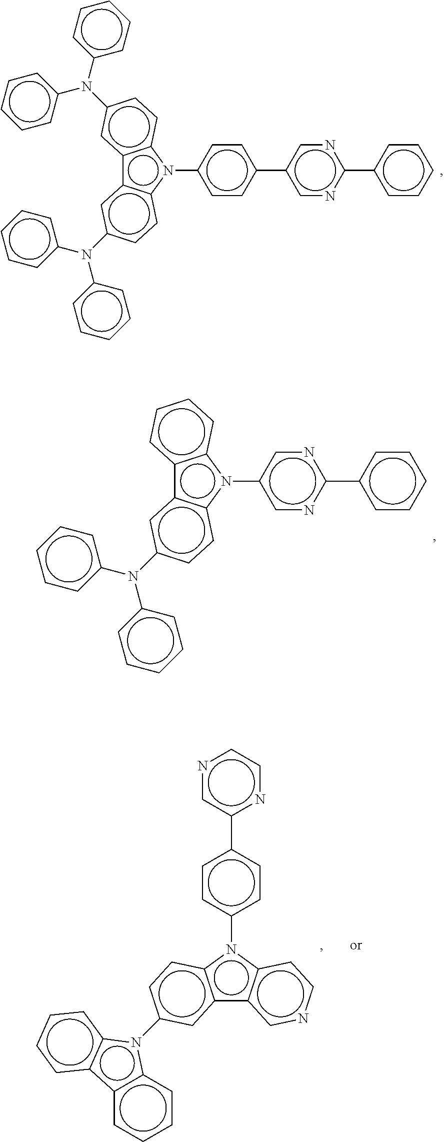

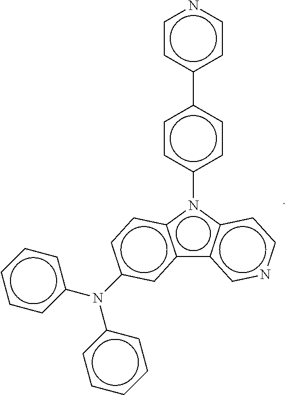

In certain embodiments of the fifteenth aspect, J is

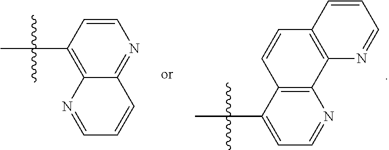 [SMILES: *c1ccnc2cccnc12 ; "or" ; *c1ccnc2c1ccc1cccnc12]

In certain embodiments, the molecule is represented by one of the following structural formulas:

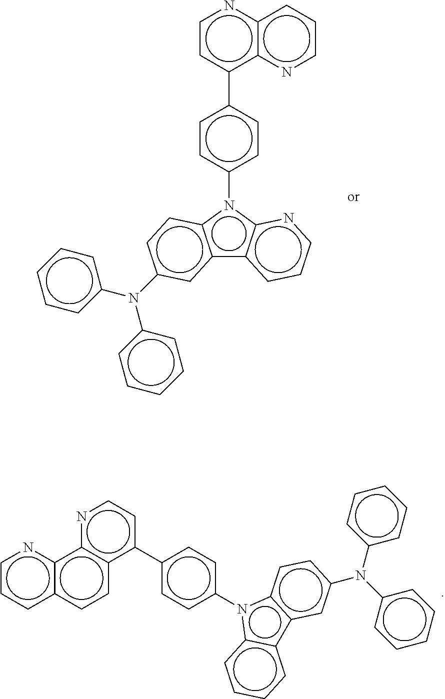

TABLE NN1

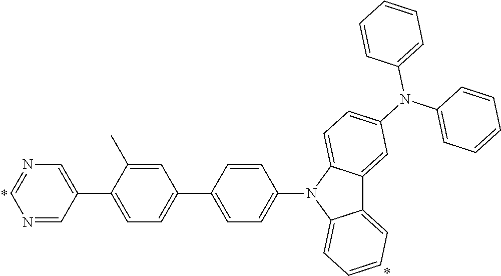

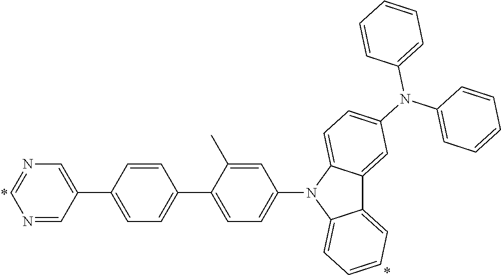

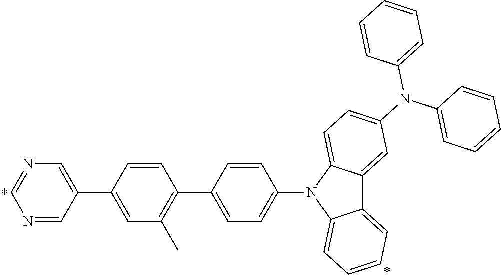

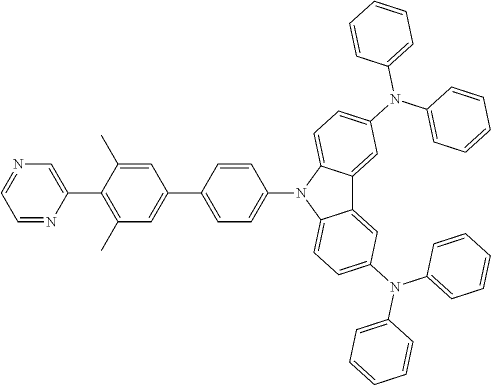 [SMILES: Cc1cc(-c2ccc(-n3c4ccc(N(c5ccccc5)c5ccccc5)cc4c4cc(N(c5ccccc5)c5ccccc5)ccc43)cc2)cc(C)c1-c1cnccn1]

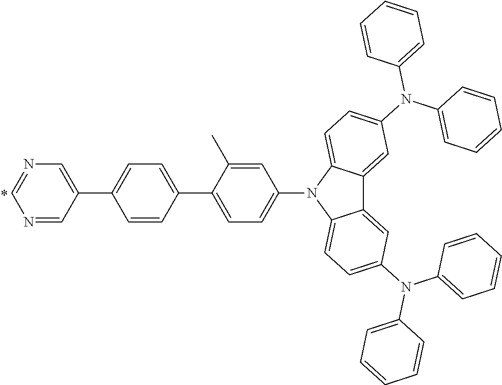

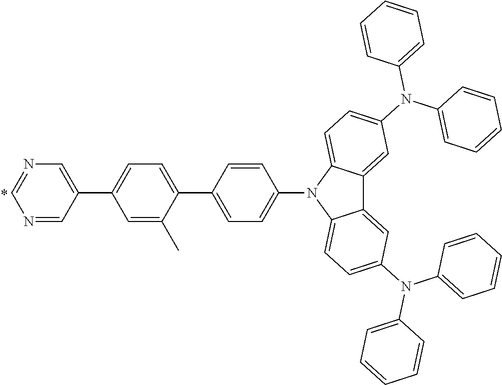

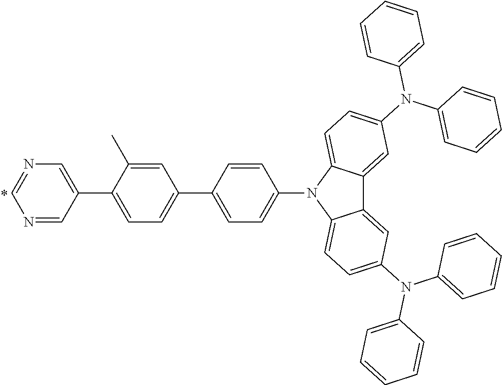

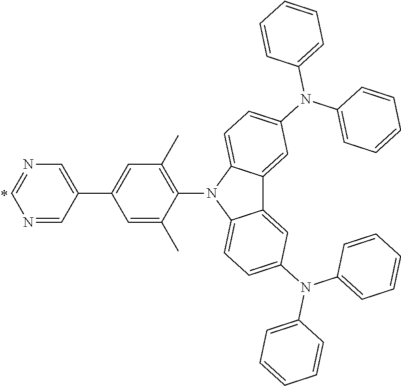

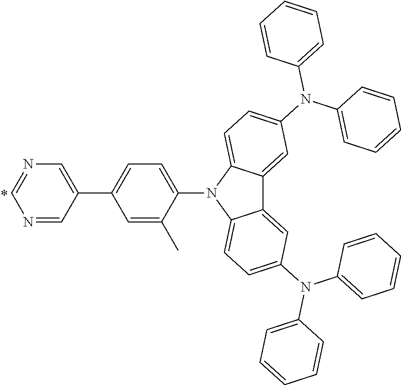

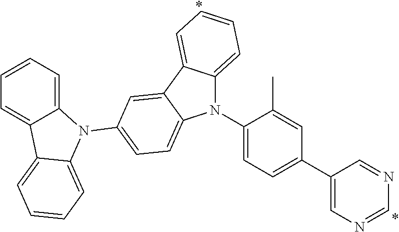

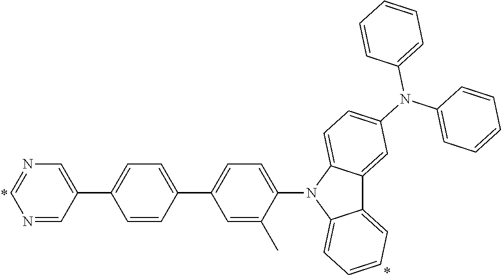

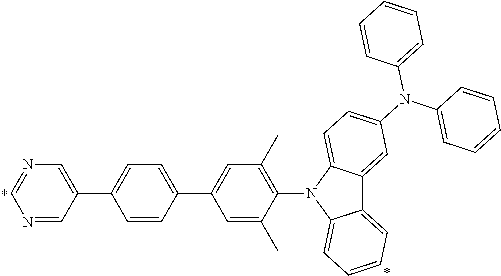

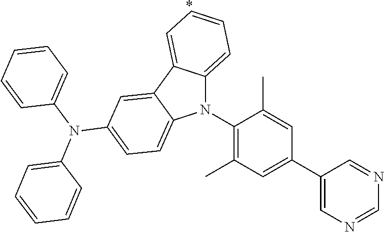

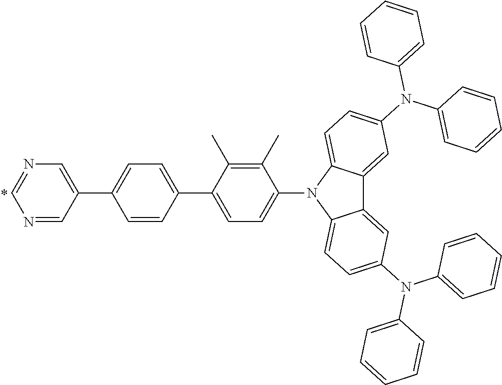

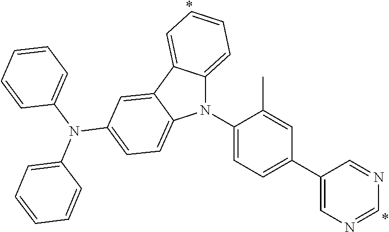

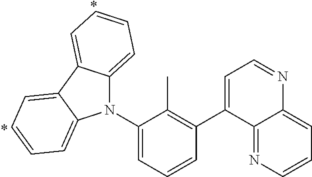

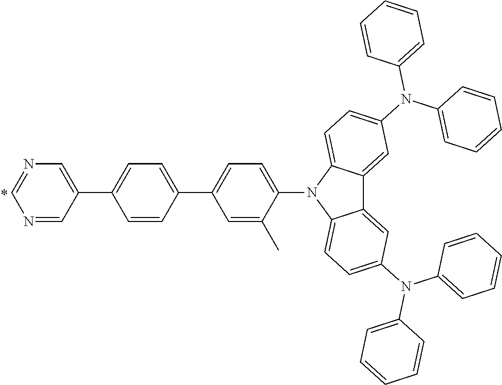

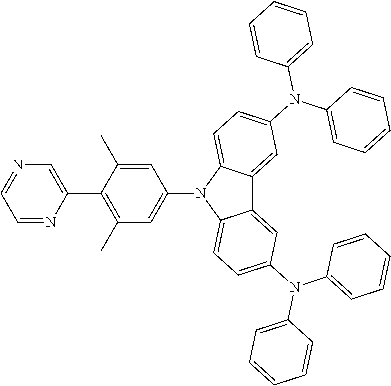 [SMILES: Cc1cc(-n2c3ccc(N(c4ccccc4)c4ccccc4)cc3c3cc(N(c4ccccc4)c4ccccc4)ccc32)cc(C)c1-c1cnccn1]

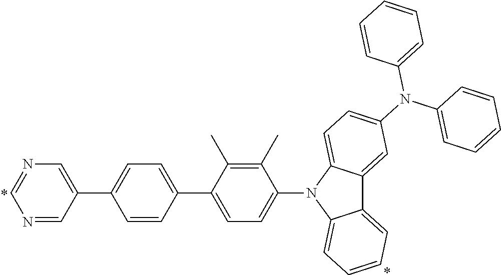

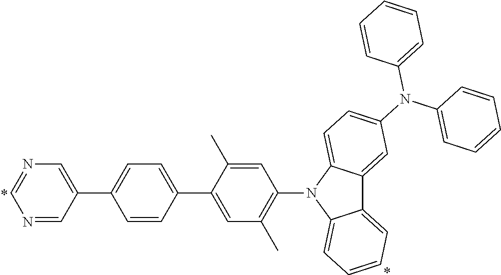

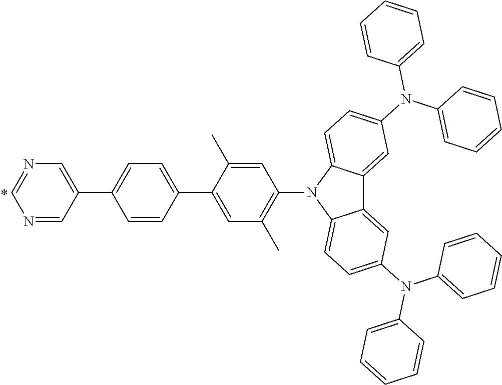

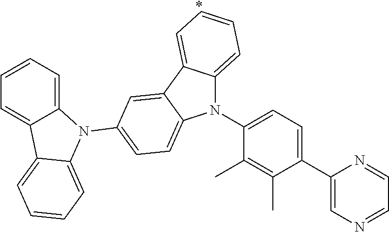

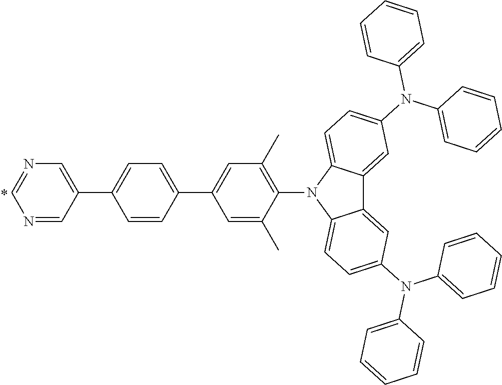

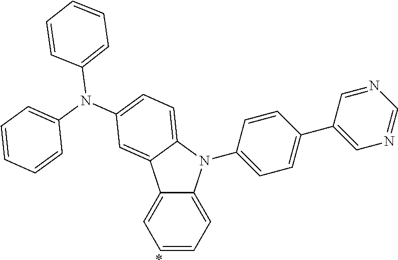

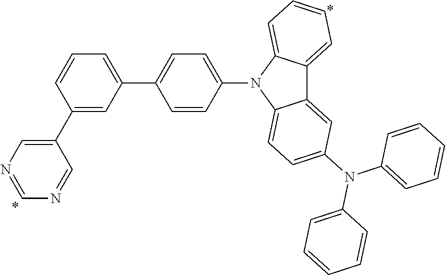

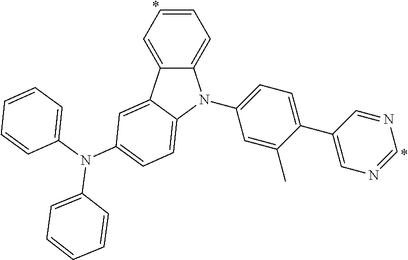

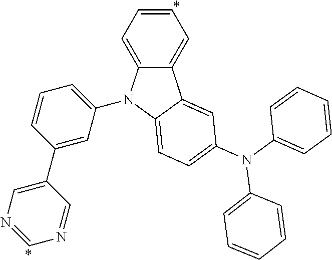

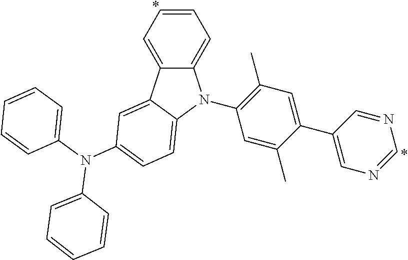

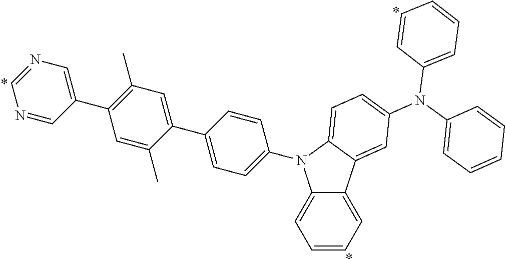

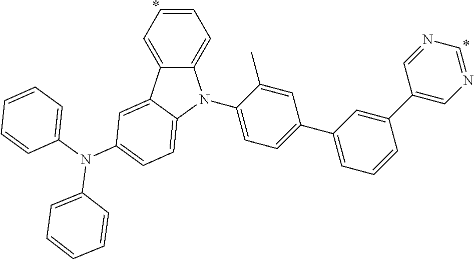

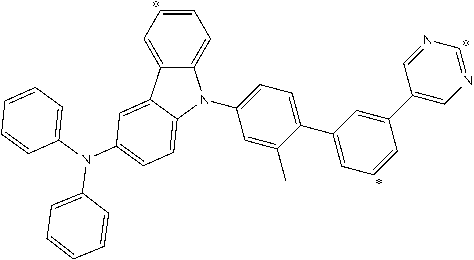

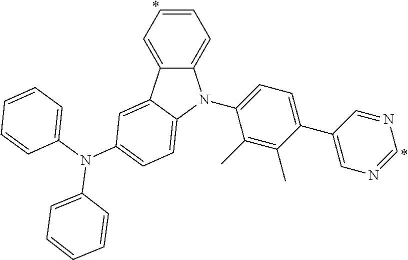

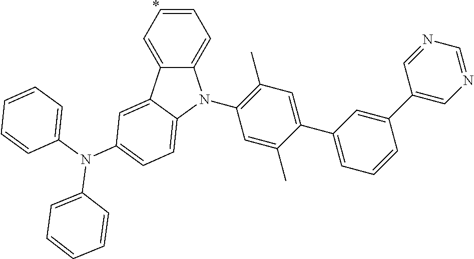

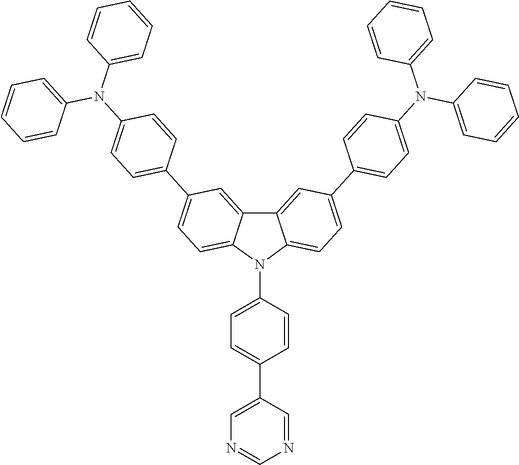 [SMILES: c1ccc(N(c2ccccc2)c2ccc(-c3ccc4c(c3)c3cc(-c5ccc(N(c6ccccc6)c6ccccc6)cc5)ccc3n4-c3ccc(-c4cncnc4)cc3)cc2)cc1]

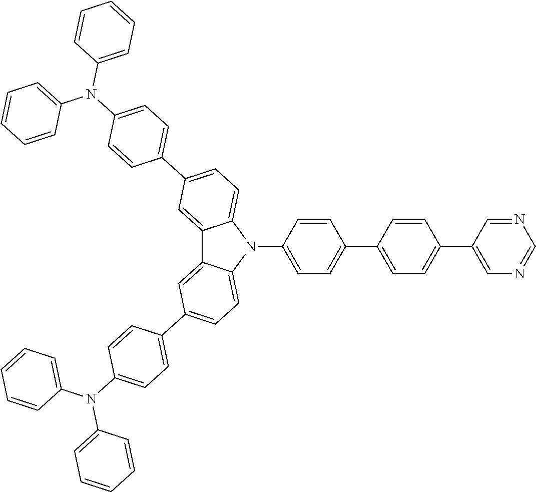 [SMILES: c1ccc(N(c2ccccc2)c2ccc(-c3ccc4c(c3)c3cc(-c5ccc(N(c6ccccc6)c6ccccc6)cc5)ccc3n4-c3ccc(-c4ccc(-c5cncnc5)cc4)cc3)cc2)cc1]

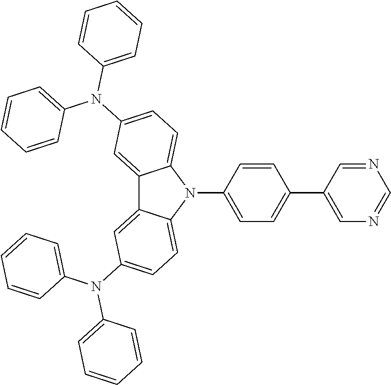 [SMILES: c1ccc(N(c2ccccc2)c2ccc3c(c2)c2cc(N(c4ccccc4)c4ccccc4)ccc2n3-c2ccc(-c3cncnc3)cc2)cc1]

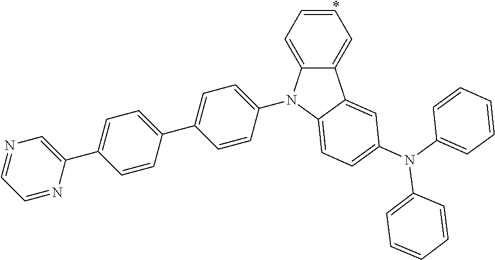

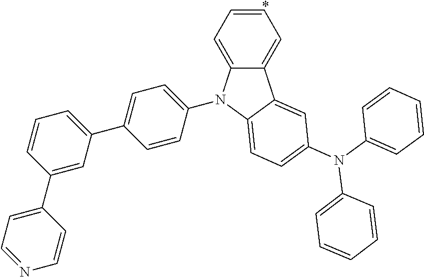

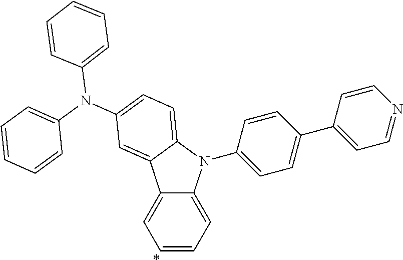

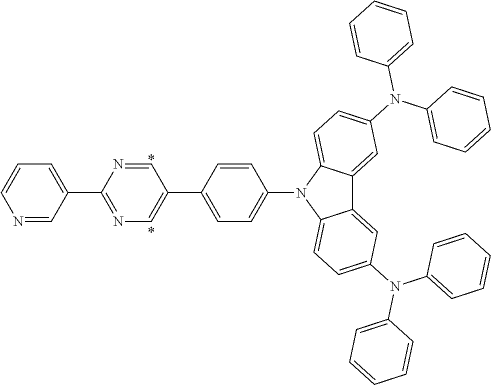

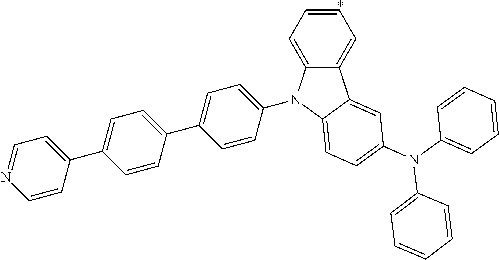

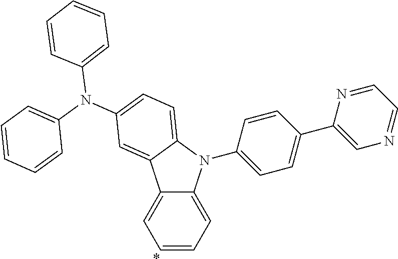

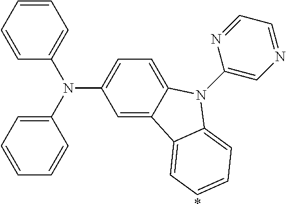

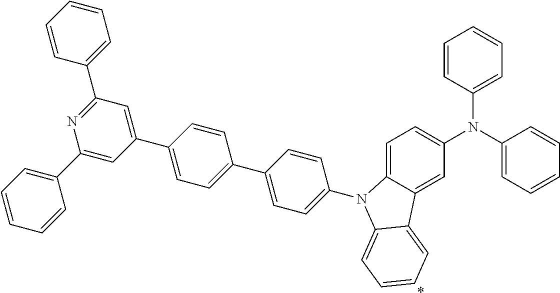

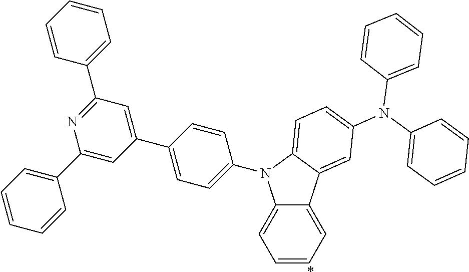

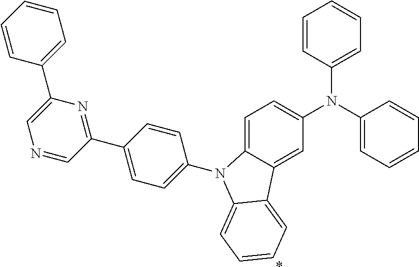

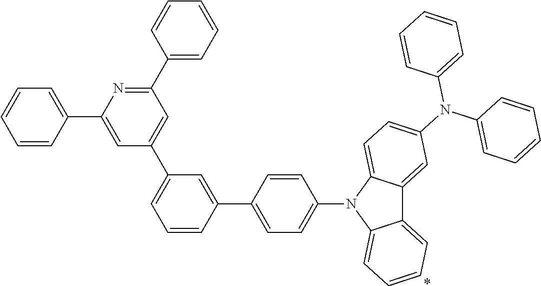

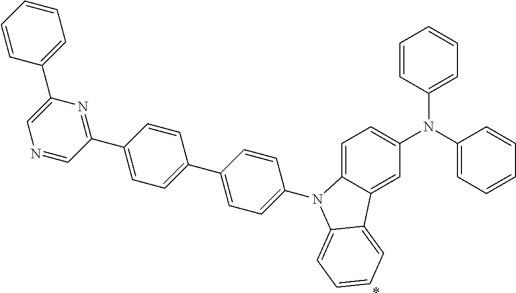

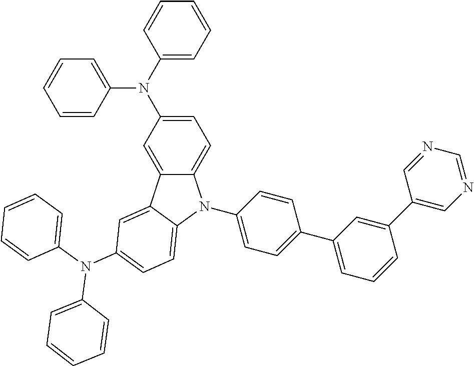 [SMILES: c1ccc(N(c2ccccc2)c2ccc3c(c2)c2cc(N(c4ccccc4)c4ccccc4)ccc2n3-c2ccc(-c3cccc(-c4cncnc4)c3)cc2)cc1]

In a sixteenth aspect, the present invention is a compound represented by structural formula (II):

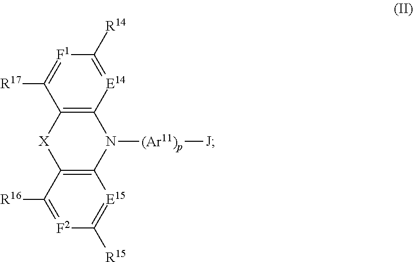

wherein:

X is O, S, or C(R^{D})₂;

R^{D}, independently for each occurrence, is a C₁-C₆ alkyl or a C₃-C₁₈ cycloalkyl;

E¹⁴, and E¹⁵, are, each independently, CR^{A} or N, wherein R^{A}, for each occurrence independently, is H, a C₁-C₆ alkyl, a C₃-C₁₈ cycloalkyl, a C₆-C₁₈ aryl, a 5-20 atom heteroaryl, a halo, or —CN;

J is any moiety selected from H,

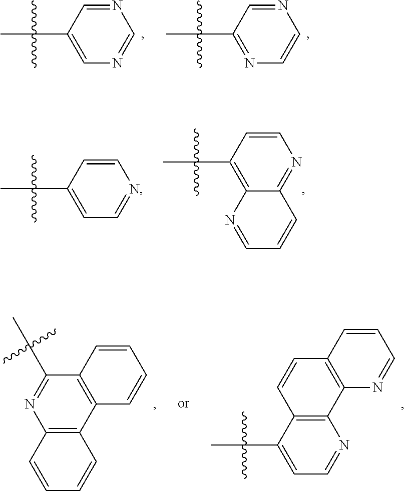

and is optionally substituted with one or more R″, each independently selected from is a C₁-C₆ alkyl, a C₃-C₁₈ cycloalkyl, a C₆-C₁₈ aryl, a 5-20 atom heteroaryl, a halo, or —CN;

R¹⁴, R¹⁵, R¹⁶, and R¹⁷ are, each independently, H, a C₁-C₆ alkyl, a C₃-C₁₈ cycloalkyl, a C₆-C₁₈ aryl, a 5-20 atom heteroaryl, a halo, or —CN;

F¹ is C—(Ar¹²)_{q}-G;

F² is CR^{B} or N, wherein R^{B} is H, a C₁-C₆ alkyl, a C₃-C₆ cycloalkyl, a C₆-C₁₈aryl, a 5-20 atom heteroaryl, a halo, or —CN, or —(Ar¹²)_{q}-G;

Ar¹¹, for each occurrence independently, is phenyl optionally substituted with one to four C₁-C₆ alkyls;

Ar¹², for each occurrence independently, is phenyl optionally substituted with one to four C₁-C₆ alkyls;

p is 0, 1, or 2;

q is 0 or 1; and

G, for each occurrence independently, is a moiety represented by one of the following structural formulas:

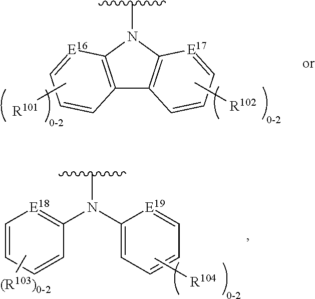

wherein:

E¹⁶, E¹⁷, E¹⁸, and B¹⁹ are, each independently, CR^{C} or N, wherein R^{C} is H, a C₁-C₃ alkyl, halo, or —CN; and

R¹⁰¹, R¹⁰², R¹⁰³, and R¹⁰⁴ are, each independently, a C₁-C₆ alkyl, a C₃-C₁₈ cycloalkyl, a C₆-C₁₈ aryl, a 5-20 atom heteroaryl, a halo, or —CN.

In certain embodiments of the sixteenth aspect, if (Ar¹¹)ₚ-J is phenyl, then F¹ and F² are other than CH. In certain embodiments of the fifteenth aspect, the present invention is not represented by any of the compounds in Table NN2. In certain embodiments of the fifteenth aspect, the molecule is not represented by any molecule represented by a structural formula in Table NN2, wherein the carbon or heteroatom denoted by (*) is unsubstituted or substituted by a C₁-C₆ alkyl, —OH , —CN, a halo, a C₆-C₁2 aryl, a 5-20 atom heteroaryl, —N(R¹⁹)₂, or —N(R²⁰)₂, wherein each R¹⁹, independently, is H or a C₁-C₆ alkyl, or a C₅-C₁₂ cycloalkyl, and wherein each R²⁰, independently, is H or a C₆-C₁₈ aryl.

In certain embodiments of the sixteenth aspect, X is O, S, or C(R^{D})₂. In certain embodiments, X is O. In certain embodiments, X is S. In certain embodiments, X is C(R^{D})₂. The remainder of the variables in structural formula (II) are as described above and below with respect to the sixteenth aspect.

In certain embodiments of the sixteenth aspect, R^{D}, independently for each occurrence, is a C₁-C₆ alkyl or a C₃-C₁₈ cycloalkyl. In certain embodiments, R^{D}, independently for each occurrence, is a C₁-C₃ alkyl. In certain embodiments, R^{D}, for each occurrence, is methyl. The remainder of the variables in structural formula (II) are as described above and below with respect to the sixteenth aspect.

In certain embodiments of the sixteenth aspect, E¹⁴, and E¹⁵ are, each independently, CR^{A} or N. In certain embodiments, E¹⁴ is CR^{A}. In certain embodiments, E¹⁴ is N. In certain embodiments, E¹⁵ is CR^{A}. In certain embodiments, E¹⁵ is N. The remainder of the variables in structural formula (II) are as described above and below with respect to the sixteenth aspect.

In certain embodiments of the sixteenth aspect, J is any moiety selected from —CN,

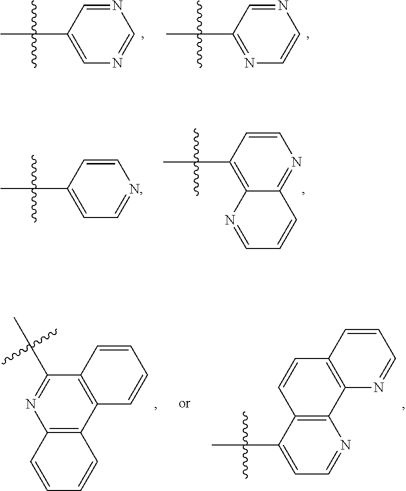

and is optionally substituted with one or more R¹¹. In certain embodiments, J is unsubstituted. In certain embodiments, J is —CN. In certain embodiments, J is

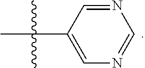 [SMILES: *c1cncnc1]

In certain embodiments, J is —CN. In certain embodiments, J is

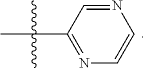 [SMILES: *c1cnccn1]

In certain embodiments, J is —CN. In certain embodiments, J is

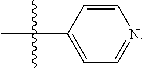 [SMILES: *c1ccncc1]

In certain embodiments, J is

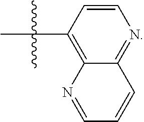 [SMILES: *c1ccnc2cccnc12]

### In

certain embodiments, J is

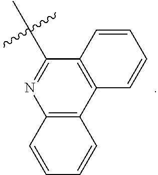 [SMILES: *c1nc2ccccc2c2ccccc12]

In certain embodiments, J is

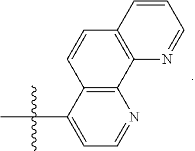 [SMILES: *c1ccnc2c1ccc1cccnc12]

The remainder of the variables in structural formula (II) are as described above and below with respect to the sixteenth aspect.

In certain embodiments of the sixteenth aspect, each R¹¹ is independently selected from C₁-C₆ alkyl, a C₃-C₁₈ cycloalkyl, a C₆-C₁₈ aryl, a 5-20 atom heteroaryl, a halo, or —CN. In certain embodiments, each is independently selected from C₁-C₆ alkyl or C₆-C₁₈ aryl. In certain embodiments, each is independently selected from C₁-C₆ alkyl or phenyl. In certain embodiments, each R¹¹ is independently selected from C₁-C₃ alkyl or phenyl. In certain embodiments, each R¹¹ is independently selected from methyl or phenyl. The remainder of the variables in structural formula (II) are as described above and below with respect to the sixteenth aspect.

In certain embodiments of the sixteenth aspect, R¹⁴ is H, a C₁-C₆ alkyl, a C₃-C₁₈ cycloalkyl, a C₆-C₁₈ aryl, a 5-20 atom heteroaryl, a halo, or —CN. In certain embodiments, R¹⁴ is H, a C₁-C₆ alkyl, a C₃-C₁₈ cycloalkyl, a halo, or —CN. In certain embodiments, R¹⁴ is H, a C₁-C₆ alkyl, or a C₃-C₆ cycloalkyl. In certain embodiments, R¹⁴ is H or methyl. In certain embodiments, R¹⁴ is H. In certain embodiments, R¹⁴ is a C₁-C₆ alkyl, a C₃-C₁₈ cycloalkyl, a C₆-C₁₈ aryl, a 5-20 atom heteroaryl, a halo, or —CN. In certain embodiments, R¹⁴ is a C₁-C₆ alkyl, a C₃-C₁₈ cycloalkyl, a halo, or —CN. In certain embodiments, R¹⁴ is a C₁-C₆ alkyl, or a C₃-C₆ cycloalkyl. In certain embodiments, R¹⁴ is methyl. The remainder of the variables in structural formula (II) are as described above and below with respect to the sixteenth aspect.

In certain embodiments of the sixteenth aspect, R¹⁵ is H, a C₁-C₆ alkyl, a C₃-C₁₈ cycloalkyl, a C₆-C₁₈ aryl, a 5-20 atom heteroaryl, a halo, or —CN. In certain embodiments, R¹⁵ is H, a C₁-C₆ alkyl, a C₃-C₁₈ cycloalkyl, a halo, or —CN. In certain embodiments, R¹⁵ is H, a C₁-C₆ alkyl, or a C₃-C₆ cycloalkyl. In certain embodiments, R¹⁵ is H or methyl. In certain embodiments, R¹⁵ is H. In certain embodiments, R¹⁵ is a C₁-C₆ alkyl, a C₃-C₁₈ cycloalkyl, a C₆-C₁₈ aryl, a 5-20 atom heteroaryl, a halo, or —CN. In certain embodiments, R¹⁵ is a C₁-C₆ alkyl, a C₃-C₁₈ cycloalkyl, a halo, or —CN. In certain embodiments, R¹⁵ is a C₁-C₆ alkyl, or a C₃-C₆ cycloalkyl. In certain embodiments, R¹⁵ is methyl. The remainder of the variables in structural formula (II) are as described above and below with respect to the sixteenth aspect.

In certain embodiments of the sixteenth aspect, R¹⁶ is H, a C₁-C₆ alkyl, a C₃-C₁₈ cycloalkyl, a C₆-C₁₈ aryl, a 5-20 atom heteroaryl, a halo, or —CN. In certain embodiments, R¹⁶ is H, a C₁-C₆ alkyl, a C₃-C₁₈ cycloalkyl, a halo, or —CN. In certain embodiments, R¹⁶ is H, a C₁-C₆ alkyl, or a C₃-C₆ cycloalkyl. In certain embodiments, R¹⁶ is H or methyl. In certain embodiments, R¹⁶ is H. In certain embodiments, R¹⁶ is a C₁-C₆ alkyl, a C₃-C₁₈ cycloalkyl, a C₆-C₁₈ aryl, a 5-20 atom heteroaryl, a halo, or —CN. In certain embodiments, R¹⁶ is a C₁-C₆ alkyl, a C₃-C₁₈ cycloalkyl, a halo, or —CN. In certain embodiments, R¹⁶ is a C₁-C₆ alkyl, or a C₃-C₆ cycloalkyl. In certain embodiments, R¹⁶ is methyl. The remainder of the variables in structural formula (II) are as described above and below with respect to the sixteenth aspect.

In certain embodiments of the sixteenth aspect, R¹⁷ is H, a C₁-C₆ alkyl, a C₃-C₁₈ cycloalkyl, a C₆-C₁₈ aryl, a 5-20 atom heteroaryl, a halo, or —CN. In certain embodiments, R¹⁷ is H, a C₁-C₆ alkyl, a C₃-C₁₈ cycloalkyl, a halo, or —CN. In certain embodiments, R¹⁷ is H, a C₁-C₆ alkyl, or a C₃-C₆ cycloalkyl. In certain embodiments, R¹⁶ is H or methyl. In certain embodiments, R¹⁷ is H. In certain embodiments, R¹⁷ is a C₁-C₆ alkyl, a C₃-C₁₈ cycloalkyl, a C₆-C₁₈ aryl, a 5-20 atom heteroaryl, a halo, or —CN. In certain embodiments, R¹⁷ is a C₁-C₆ alkyl, a C₃-C₁₈ cycloalkyl, a halo, or —CN. In certain embodiments, R¹⁷ is a C₁-C₆ alkyl, or a C₃-C₆ cycloalkyl. In certain embodiments, R¹⁷ is methyl. The remainder of the variables in structural formula (II) are as described above and below with respect to the sixteenth aspect.

In certain embodiments of the sixteenth aspect, F¹ is C—(Ar¹²)_{q}-G. The remainder of the variables in structural formula (II) are as described above and below with respect to the sixteenth aspect.

In certain embodiments of the sixteenth aspect, F² is N or CR^{B}. In certain embodiments, F² is CR^{B}. In certain embodiments, F² is N. The remainder of the variables in structural formula (II) are as described above and below with respect to the sixteenth aspect.

In certain embodiments of the sixteenth aspect, each R^{B} is, independently, H, a C₁-C₆ alkyl, a C₃-C₆ cycloalkyl, a C₆-C₁₈ aryl, a 5-20 atom heteroaryl, a halo, or —CN, or —(Ar¹²)_{q}-G. In certain embodiments, each R^{B} is, independently, H, a C₁-C₆ alkyl, a C₃-C₆ cycloalkyl, or —(Ar¹²)_{q}-G. In certain embodiments, each R^{B} is, independently, H, a C₁-C₆ alkyl, a C₃-C₆ cycloalkyl, or a moiety represented by one of the following structural formulas:

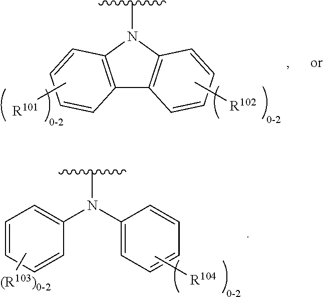

In certain embodiments, each R^{B} is, independently, H or a moiety represented by the following structural formula:

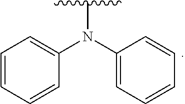 [SMILES: *N(c1ccccc1)c1ccccc1]

In certain embodiments, each R^{B} is, independently, H or a moiety represented by the following structural formula:

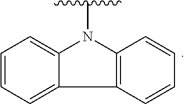 [SMILES: *n1c2ccccc2c2ccccc21]

In certain embodiments, each R^{B} is, independently, H or a moiety represented by the following structural formula:

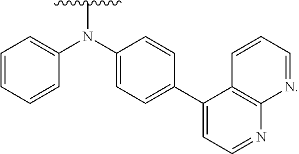 [SMILES: *N(c1ccccc1)c1ccc(-c2ccnc3ncccc23)cc1]

The remainder of the variables in structural formula (II) are as described above and below with respect to the sixteenth aspect.

In certain embodiments of the sixteenth aspect, Ar¹¹, for each occurrence independently, is phenyl optionally substituted with one to four C₁-C₆ alkyls. In certain embodiments, Ar¹¹, for each occurrence independently, is phenyl optionally substituted with one to four C₁-C₃ alkyls. In certain embodiments, Ar¹¹, for each occurrence independently, is phenyl optionally substituted with one to four methyls. In certain embodiments, Ar¹¹, for each occurrence, is unsubstituted phenyl. In certain embodiments, Ar¹¹, for each occurrence independently, is phenyl optionally substituted with one to four C₂-C₆ alkyls. The remainder of the variables in structural formula (II) are as described above and below with respect to the sixteenth aspect.

In certain embodiments of the sixteenth aspect, Ar¹², for each occurrence independently, is phenyl optionally substituted with one to four C₁-C₆ alkyls. In certain embodiments, Ar¹², for each occurrence independently, is phenyl optionally substituted with one to four C₁-C₃ alkyls. In certain embodiments, Ar¹², for each occurrence independently, is phenyl optionally substituted with one to four methyls. In certain embodiments, Ar¹², for each occurrence, is unsubstituted phenyl. In certain embodiments, Ar¹², for each occurrence independently, is phenyl optionally substituted with one to four C₂-C₆ alkyls. The remainder of the variables in structural formula (II) are as described above and below with respect to the sixteenth aspect.

In certain embodiments of the sixteenth aspect, p is 0, 1, or 2. In certain embodiments, p is 0. In certain embodiments, p is 1. In certain embodiments, p is 2. The remainder of the variables in structural formula (II) are as described above and below with respect to the sixteenth aspect.

In certain embodiments of the sixteenth aspect, q is 0 or 1. In certain embodiments, q is 0. In certain embodiments, q is 1. The remainder of the variables in structural formula (II) are as described above and below with respect to the sixteenth aspect.

In certain embodiments of the sixteenth aspect, G, for each occurrence independently, is a moiety represented by one of the following structural formulas:

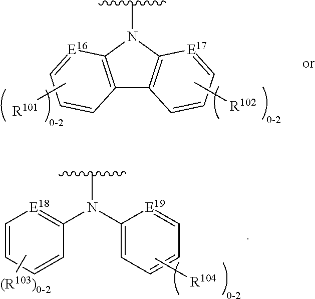

In certain embodiments, G is a moiety represented by one of the following structural formulas:

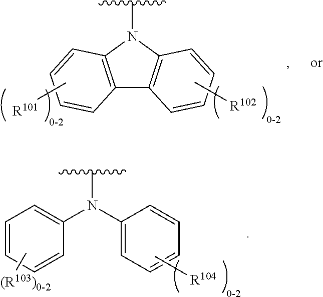

In certain embodiments, G is a moiety represented the following structural formula:

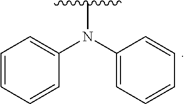 [SMILES: *N(c1ccccc1)c1ccccc1]

In certain embodiments, G is a moiety represented the following structural formula:

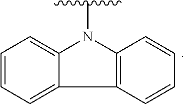 [SMILES: *n1c2ccccc2c2ccccc21]

In certain embodiments, G is a moiety represented the following structural formula:

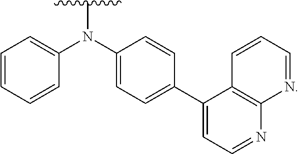 [SMILES: *N(c1ccccc1)c1ccc(-c2ccnc3ncccc23)cc1]

The remainder of the variables in structural formula (II) are as described above and below with respect to the sixteenth aspect.

In certain embodiments of the sixteenth aspect, E¹⁶ is CR^{C} or N. In certain embodiments, E¹⁶ is CR^{C}. In certain embodiments, E¹⁶ is N. The remainder of the variables in structural formula (II) are as described above and below with respect to the sixteenth aspect.

In certain embodiments of the sixteenth aspect, E¹⁷ is CR^{C} or N. In certain embodiments, E¹⁷ is CR^{C}. In certain embodiments, E¹⁷ is N. The remainder of the variables in structural formula (II) are as described above and below with respect to the sixteenth aspect.

In certain embodiments of the sixteenth aspect, E¹⁸ is CR^{C} or N. In certain embodiments, E¹⁸ is CR^{C}. In certain embodiments, E¹⁸ is N. The remainder of the variables in structural formula (II) are as described above and below with respect to the sixteenth aspect.

In certain embodiments of the sixteenth aspect, E¹⁹ is CR^{C} or N. In certain embodiments, E¹⁹ is CR^{C}. In certain embodiments, E¹⁹ is N. The remainder of the variables in structural formula (II) are as described above and below with respect to the sixteenth aspect.

In certain embodiments of the sixteenth aspect, each R^{C} is, independently, H, a C₁-C₃ alkyl, halo, or —CN. In certain embodiments, each R^{C} is H. The remainder of the variables in structural formula (II) are as described above and below with respect to the sixteenth aspect.

In certain embodiments of the sixteenth aspect, each R¹⁰² is, independently, a C₁-C₆ alkyl, a C₃-C₁₈ cycloalkyl, a C₆-C₁₈ aryl, a 5-20 atom heteroaryl, a halo, or —CN. In certain embodiments, each R¹⁰² is, independently, a C₁-C₆ alkyl or a C₃-C₆ cycloalkyl. The remainder of the variables in structural formula (II) are as described above and below with respect to the sixteenth aspect.

In certain embodiments of the sixteenth aspect, each R¹⁰² is, independently, a C₁-C₆ alkyl, a C₃-C₁₈ cycloalkyl, a C₆-C₁₈ aryl, a 5-20 atom heteroaryl, a halo, or —CN. In certain embodiments, each R¹⁰² is, independently, a C₁-C₆ alkyl or a C₃-C₆ cycloalkyl. The remainder of the variables in structural formula (II) are as described above and below with respect to the sixteenth aspect.

In certain embodiments of the sixteenth aspect, each R¹⁰³ is, independently, a C₁-C₆ alkyl, a C₃-C₁₈ cycloalkyl, a C₆-C₁₈ aryl, a 5-20 atom heteroaryl, a halo, or —CN. In certain embodiments, each R¹⁰³ is, independently, a C₁-C₆ alkyl or a C₃-C₆ cycloalkyl. The remainder of the variables in structural formula (II) are as described above and below with respect to the sixteenth aspect.

In certain embodiments of the sixteenth aspect, each R¹⁰⁴ is, independently, a C₁-C₆ alkyl, a C₃-C₁₈ cycloalkyl, a C₆-C₁₈ aryl, a 5-20 atom heteroaryl, a halo, or —CN. In certain embodiments, each R¹⁰⁴ is, independently, a C₁-C₆ alkyl or a C₃-C₆ cycloalkyl. The remainder of the variables in structural formula (II) are as described above and below with respect to the sixteenth aspect.

In certain embodiments of the sixteenth aspect:

R^{A}, for each occurrence independently, is H or a C₁-C₆ alkyl;

R¹⁴, R¹⁵, R¹⁶, and R¹⁷ are, each independently, H, a C₁-C₆ alkyl, or a C₃-C₆ cycloalkyl; F² is CR^{B}; and

R^{B} is H, a C₁-C₆ alkyl, a C₃-C₆ cycloalkyl, or —(Ar¹²)_{q}-G.

The remainder of the variables in structural formula (II) are as described above and below with respect to the sixteenth aspect.
In certain embodiments of the sixteenth aspect, the molecule is represented by the following structural formula:

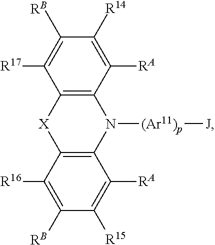

wherein the variables in structural formula (II) are as described above and below with respect to the sixteenth aspect. In certain embodiments:

R^{A}, for each occurrence independently, is H or a C₁-C₆ alkyl;

R^{D}, for each occurrence, is methyl;

p is 0 or 1;

R¹¹ is a C₆-C₁₈ aryl or a 5-20 atom heteroaryl;

R¹⁴, R¹⁵, R¹⁶, and R¹⁷ are, each independently, H, a C₁-C₆ alkyl, a C₃-C₁₈ cycloalkyl, a halo, or —CN;

R^{B} is, for each occurrence independently, H, a C₁-C₆ alkyl, a C₃-C₆ cycloalkyl, or a moiety represented by one of the following structural formulas:

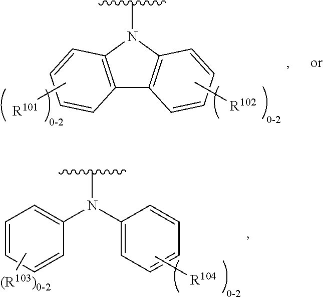

R¹⁰¹, R¹⁰², R¹⁰³, and R¹⁰⁴ are, each independently, a C₁-C₆ alkyl, a C₃-C₁₀ cycloalkyl, a C₆-C₁₀ aryl, or a 5-10 atom heteroaryl.

In certain embodiments of the sixteenth aspect, the molecule is represented by one of the following structural formulas:

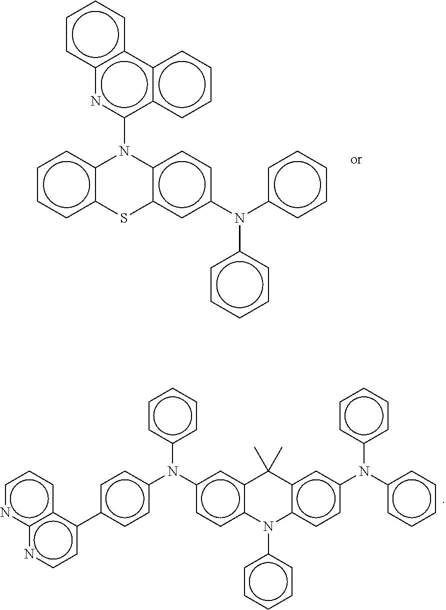

TABLE NN2

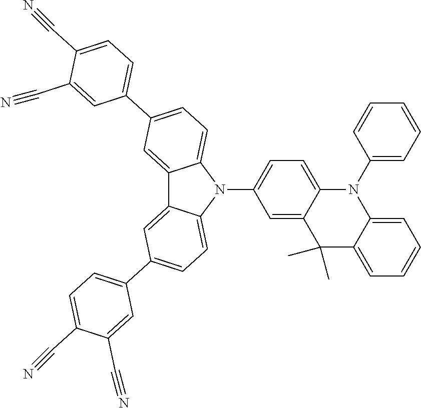 [SMILES: CC1(C)c2ccccc2N(c2ccccc2)c2ccc(-n3c4ccc(-c5ccc(C#N)c(C#N)c5)cc4c4cc(-c5ccc(C#N)c(C#N)c5)ccc43)cc21]

In a seventeenth aspect, the present invention is a compound represented by one of structural formulas (IIIA), (IIIB), (IIIC), (IIID), or (IIIE):

PA (IIIA)

PAP (IIIB)

PPA (IIIC)

PAA (IIID)

APA (IIIE)

In structural formulas (IIIA)-(IIIE):

The moieties P and A are either covalently linked or are linked by a moiety φ;

P is represented by the following structural formula:

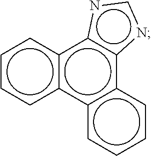

each instance of P, independently, is optionally substituted with one or more groups R³¹, each independently selected from a C₁-C₆ alkyl, a C₃-C₁₈ cycloalkyl, a C₆-C₁₈ aryl, a 5-20 atom heteroaryl, a halo, or —CN;

each instance of P is, independently, linked to the remainder of the molecule by any one atom in the heterocyclic ring portion;

each instance of φ, independently, phenyl optionally substituted with one to four C₁-C₆ alkyls; and

A is a 5-20 atom heteroaryl, optionally substituted with one or more groups R³², each independently selected from a C₁-C₆ alkyl, a C₃-C₁₈ cycloalkyl, a C₆-C₁₈ aryl, a 5-20 atom heteroaryl, a halo, or —CN.

In certain embodiments of the seventeenth aspect, P is represented by the following structural formula:

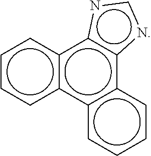

In certain embodiments, each instance of P, independently, is optionally substituted with one or more R³¹. In certain embodiments, each instance of P is unsubstituted. The remainder of the variables in structural formulas (IIIA)-(IIIE)are as defined above and below with respect to the seventeenth aspect.

In certain embodiments of the seventeenth aspect, each instance of φ, independently, is phenyl optionally substituted with one to four C₁-C₆ alkyls. In certain embodiments, each instance of φ, independently, is phenyl optionally substituted with one to four C₁-C₃ alkyls. In certain embodiments, each instance of φ, independently, is phenyl optionally substituted with one to four methyls. In certain embodiments, each instance of φ, independently, is unsubstituted phenyl. The remainder of the variables in structural formulas (IIIA)-(IIIE) are as defined above and below with respect to the seventeenth aspect.

In certain embodiments of the seventeenth aspect, each instance of A, independently, is a 5-20 atom heteroaryl, optionally substituted with one or more R³². In certain embodiments, each instance of A, independently, is pyridinyl, pyrimidinyl, triazinyl, quinoline, isoquinoline, or a diazanaphthalene. In certain embodiments, each instance of A, independently, is triazinyl or 1,4-diazanaphthalene. In certain embodiments, each instance of A, independently, is 1,4-diazanaphthalene. In certain embodiments, each instance of A, independently, is triazinyl. In certain embodiments, each instance of A, independently, is unsubstituted. The remainder of the variables in structural formulas (IIIA)-(IIIE)are as defined above and below with respect to the seventeenth aspect.

In certain embodiments of the seventeenth aspect, each instance of R³¹ is independently selected from φ, a C₁-C₆ alkyl, a C₃-C₁₈ cycloalkyl, a C₆-C₁₈ aryl, a 5-20 atom heteroaryl, a halo, or —CN. In certain embodiments, each instance of R³¹ is independently selected from φ, C₁-C₆ alkyl, a C₃-C₁₈ cycloalkyl, a halo, or —CN. In certain embodiments, each instance of R³¹ is independently selected from φ or methyl. In certain embodiments, each instance of R³¹ is independently selected from φ. The remainder of the variables in structural formulas (IIIA)-(IIIE)are as defined above and below with respect to the seventeenth aspect.

In certain embodiments of the seventeenth aspect, each instance of R³² is independently selected from φ, a C₁-C₆ alkyl, a C₃-C₁₈ cycloalkyl, a C₆-C₁₈ aryl, a 5-20 atom heteroaryl, a halo, or —CN. In certain embodiments, each instance of R³² is independently selected from φ, C₁-C₆ alkyl, a C₃-C₁₈ cycloalkyl, a halo, or —CN. In certain embodiments, each instance of R³² is independently selected from φ or methyl. In certain embodiments, each instance of R³² is independently selected from φ. The remainder of the variables in structural formulas (IIIA)-(IIIE) are as defined above and below with respect to the seventeenth aspect.

In certain embodiments of the seventeenth aspect, the molecule is represented by structural formula (IIIA). The variables are as defined above with respect to the seventeenth aspect. In certain embodiments, the molecule is represented by the following structural formula:

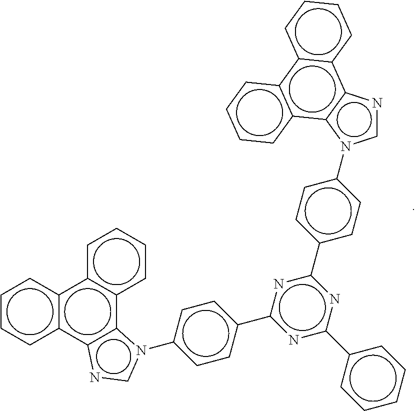

In certain embodiments of the seventeenth aspect, the molecule is represented by structural formula (IIIB). The variables are as defined above with respect to the seventeenth aspect. In certain embodiments, the molecule is represented by the following structural formula:

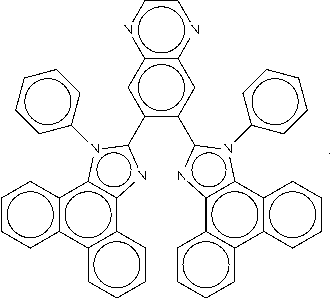

In certain embodiments of the seventeenth aspect, the molecule is represented by structural formula (IIIC). The variables are as defined above with respect to the seventeenth aspect. In certain embodiments, the molecule is represented by the following structural formula:

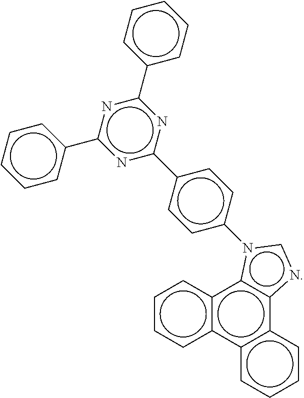

In an eighteenth aspect, the present invention is a compound represented by structural formula (IV):

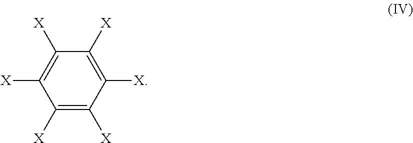

In structural formula (IV):

each X is, independently, selected from H, C₁-C₆ alkyl, a C₃-C₁₈ cycloalkyl, a C₆-C₁₈ aryl, a 5-20 atom heteroaryl, halo, —CN, or —(Ar²¹)_{d}-G;

d, for each occurrence independently, is 0, 1, or 2;

Ar²¹, for each occurrence independently, is phenyl optionally substituted with one to four C₁-C₆ alkyls;

G, for each occurrence independently, is C₆-C₁₈ aryl or 5-20 atom heteroaryl. In certain embodiments, at least one instance of X is —(Ar²¹)_{d}-G⁴, wherein G⁴ is benzothiophene. In certain embodiments, at least one instance of X is —CN. In certain embodiments, the molecule is not represented by the following structural formula:

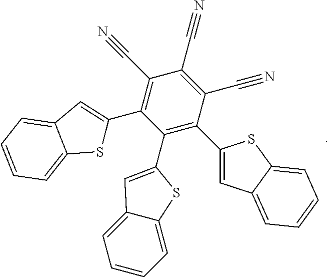 [SMILES: N#Cc1c(C#N)c(-c2cc3ccccc3s2)c(-c2cc3ccccc3s2)c(-c2cc3ccccc3s2)c1C#N]

In certain embodiments of the eighteenth aspect, each X is, independently, selected from H, C₁-C₆ alkyl, a C₃-C₁₈ cycloalkyl, a C₆-C₁₈ aryl, a 5-20 atom heteroaryl, halo, —CN, or —(Ar²¹)_{d}-G. In certain embodiments, each X is, independently, selected from C₁-C₆ alkyl, a C₃-C₁₈ cycloalkyl, a C₆-C₁₈ aryl, a 5-20 atom heteroaryl, halo, —CN, or —(Ar²¹)_{d}-G. In certain embodiments, each X is, independently, selected from H, —CN, or —(Ar²¹)_{d}-G. In certain embodiments, at least two instances of X are —(Ar²¹)_{d}-G⁴. In certain embodiments, at least three instances of X are —(Ar²¹)_{d}-G⁴. In certain embodiments, at least four instances of X are —(Ar²¹)_{d}-G⁴. In certain embodiments, at least two instances of X are —CN. In certain embodiments, at least three instances of X are —CN. In certain embodiments, at least four instances of X are —CN. In certain embodiments, three instances of X are —(Ar²¹)_{d}-G⁴ and three instances of X are —CN. The remainder of the variables in structural formula (IV) are as defined above and below with respect to the eighteenth aspect.

In certain embodiments of the eighteenth aspect, d, for each occurrence independently, is 0, 1, or 2. In certain embodiments, d, for each occurrence independently, is 0 or 1. In certain embodiments, d, for each occurrence independently, is 0. In certain embodiments, d, for each occurrence independently, is 1. In certain embodiments, d, for each occurrence independently, is 2. The remainder of the variables in structural formula (IV) are as defined above and below with respect to the eighteenth aspect.

In certain embodiments of the eighteenth aspect, Ar²¹, for each occurrence independently, is phenyl optionally substituted with one to four C₁-C₆ alkyls. In certain embodiments, Ar²¹, for each occurrence independently, is phenyl optionally substituted with one to four C₁-C₃ alkyls. In certain embodiments, Ar²¹, for each occurrence independently, is phenyl optionally substituted with one to four methyls. In certain embodiments, Ar²¹, for each occurrence, is unsubstituted phenyl. In certain embodiments, Ar²¹, for each occurrence independently, is phenyl optionally substituted with one to four C₂-C₆ alkyls. The remainder of the variables in structural formula (IV) are as defined above and below with respect to the eighteenth aspect.

In certain embodiments of the eighteenth aspect, G, for each occurrence independently, is C₆-C₁₈ aryl or 5-20 atom heteroaryl. In certain embodiments, G is benzothiophene. In certain embodiments, G is represented by the following structural formula:

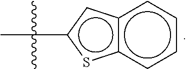

The remainder of the variables in structural formula (IV) are as defined above and below with respect to the eighteenth aspect.

In certain embodiments of the eighteenth aspect, the molecule is represented by the following structural formula:

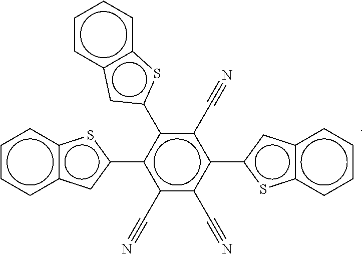

In a nineteenth aspect, the present invention is a compound represented by structural formula (VA), (VB), or (VC):

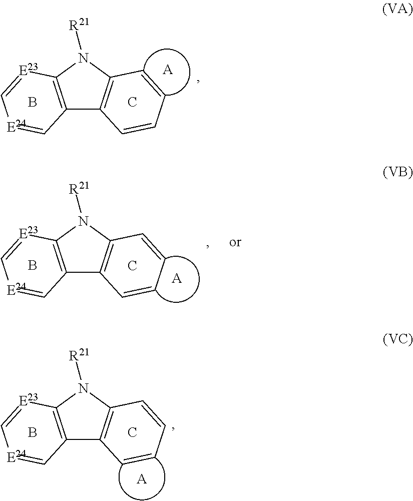

According to structural formulas (VA), (VB), and (VC):

Ring A, for each occurrence independently, is represented by the following structural formula:

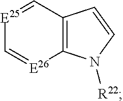

Rings A, B, and C, each independently, are optionally substituted with 1 or 2 substituents selected from a C₁-C₆ alkyl, a C₃-C₁₈ cycloalkyl, a C₆-C₁₈ aryl, a 5-20 atom heteroaryl, halo, or —CN;

R²¹ and R²², each independently, are selected from H, a C₁-C₆ alkyl, a C₃-C₁₈ cycloalkyl, a C₆-C₁₈ aryl, a 5-20 atom heteroaryl, halo, —CN, —(Ar²¹)_{d}-G, or —Ar²², provided that at least one of R²¹ and R²² is —(Ar²¹)_{d}-G or —(Ar²¹)_{d}—Ar²²;

Ar²¹, for each occurrence independently, is phenyl optionally substituted with one to four C₁-C₆ alkyls;

Ar²², for each occurrence independently, is:

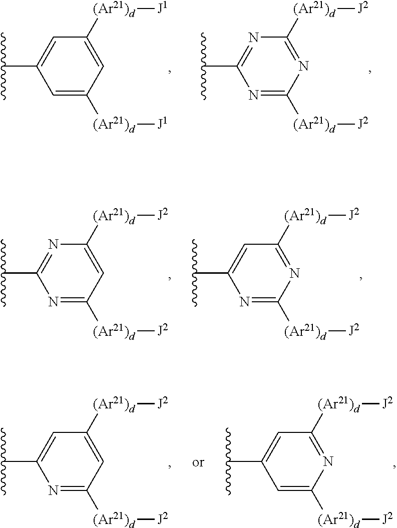

and is optionally substituted with one to three C₁-C₆ alkyls;

d, for each occurrence independently, is 0, 1, or 2;

J¹, for each occurrence independently, is H, C₆-C₁₈ aryl or 5-20 atom heteroaryl and is optionally substituted by one or more —CN, —C(O)phenyl, C₁-C₆ alkyl, C₁-C₆ haloalkyl, C₆-C₁₈ aryl, or (5-6 atom) heteroaryl, provided that if at least one instance of Ar²² is

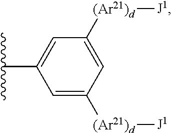

then at least one J¹ is not H or unsubstituted phenyl;

J², for each occurrence independently, is H, C₆-C₁₈ aryl or 5-20 atom heteroaryl and is optionally substituted by one or more —CN, —C(O)phenyl, C₁-C₆ alkyl, C₁-C₆ haloalkyl, C₆-C₁₈ aryl, or (5-6 atom) heteroaryl;

G, for each occurrence independently, is C₆-C₁₈ aryl or 5-20 atom heteroaryl, provided that G is not triazinyl, pyrimidinyl, tetrazolyl, oxadiazolyl, diazanaphthyl, or pentafluorophenyl, further provided that if G is phenyl it is not unsubstituted and is not substituted with carbonyl, trifluoromethyl, triazinyl, pyrimidinyl, tetrazolyl, oxadiazolyl, diazanaphthyl, or pentafluorophenyl; and

E²³, E²⁴, E²⁵, and E²⁶ are, each independently, CR^{Y} or N, wherein R^{Y}, for each occurrence independently, is H, a C₁-C₆ alkyl, a C₃-C₁₈ cycloalkyl, a C₆-C₁₈ aryl, a 5-20 atom heteroaryl, halo, or —CN.

In certain embodiments of the nineteenth aspect, the compound is not represented by any structural formula in Table NR. In certain embodiments of the fifteenth aspect, the molecule is not represented by any molecule represented by a structural formula in Table NR, wherein the carbon or heteroatom denoted by (*) is unsubstituted or substituted by a C₁-C₆ alkyl, —OH , —CN, a halo, a C₆-C₁2 aryl, a 5-20 atom heteroaryl, —N(R¹⁹)₂, or —N(R²⁰)₂, wherein each R¹⁹, independently, is H or a C₁-C₆ alkyl, or a C₅-C₁₂ cycloalkyl, and wherein each R²⁰, independently, is H or a C₆-C₁₈ aryl.

In certain embodiments of the nineteenth aspect, the molecule is represented by any one of structural formulas (VD), (VE), or (VF):

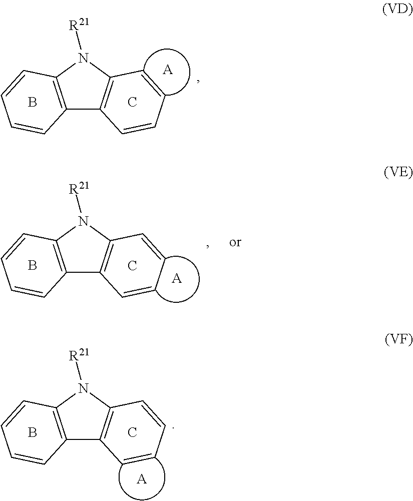

In structural formulas (VD), (VE), and (VF):

Ring A, for each occurrence independently, is represented by the following structural formula:

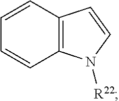

and rings A, B, and C, each independently, are optionally substituted with 1 to 4 substituents selected from a C₁-C₆ alkyl, a C₃-C₁₈ cycloalkyl, a C₆-C₁₈ aryl, a 5-20 atom heteroaryl, halo, or —CN. The remainder of the variables in structural formulas (VD), (VE), and (VF) are as defined above and below with respect to the nineteenth aspect.

In certain embodiments of the nineteenth aspect, the molecule is represented by structural formula (VG):

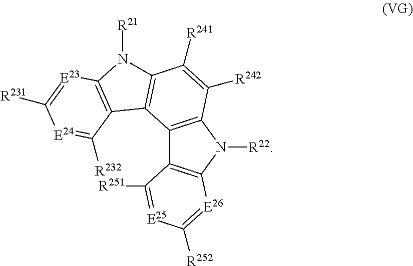

In structural formula (VG), R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN. The remainder of the variables in structural formula (VG) are as defined above and below with respect to the nineteenth aspect.

In certain embodiments of the nineteenth aspect, the molecule is represented by structural formula (VH):

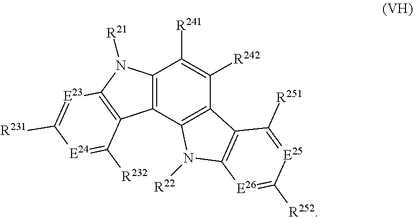

In structural formula (VH), R²³¹, R²³², R²⁴¹, R²⁴², R²⁵¹, and R²⁵², and R²⁵², each independently, are H, a C₁-C₆ alkyl, a C₃-C₁₈ cycloalkyl, a C₆-C₁₈ aryl, a 5-20 atom heteroaryl, halo, or —CN. The remainder of the variables in structural formula (VH) are as defined above and below with respect to the nineteenth aspect.

In certain embodiments of the nineteenth aspect, the molecule is represented by structural formula (VJ):

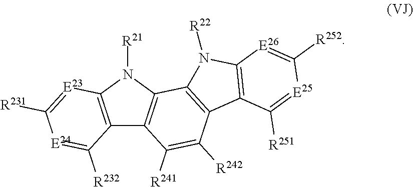

In structural formula (VJ), R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN. The remainder of the variables in structural formula (VJ) are as defined above and below with respect to the nineteenth aspect.

In certain embodiments of the nineteenth aspect, the molecule is represented by structural formula (VK):

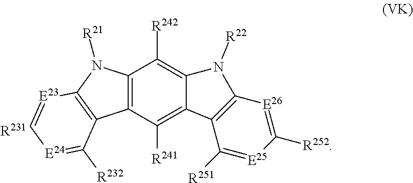

In structural formula (VK), R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN. The remainder of the variables in structural formula (VK) are as defined above and below with respect to the nineteenth aspect.

In certain embodiments of the nineteenth aspect, the molecule is represented by structural formula (VL):

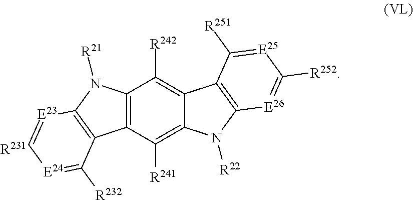

In structural formula (VL), R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN. The remainder of the variables in structural formula (VL) are as defined above and below with respect to the nineteenth aspect.

In certain embodiments of the nineteenth aspect, ring A may be fused to ring C in any orientation. In certain embodiments, any two atoms of the six-membered ring in ring A may be shared with ring C. In other embodiments, the two carbon atoms of the five-membered ring ing ring A may be shared with ring C. In example embodiments, the compounds of structures (VA), (VB), and (VC) can be represented by the following structural formulas:

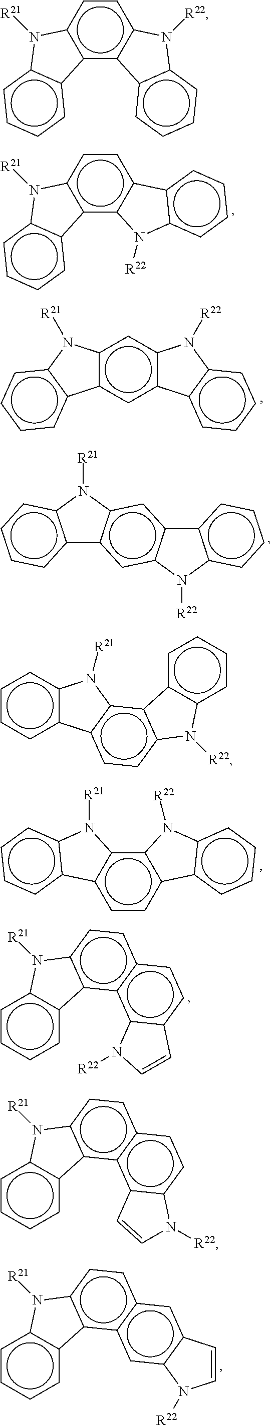

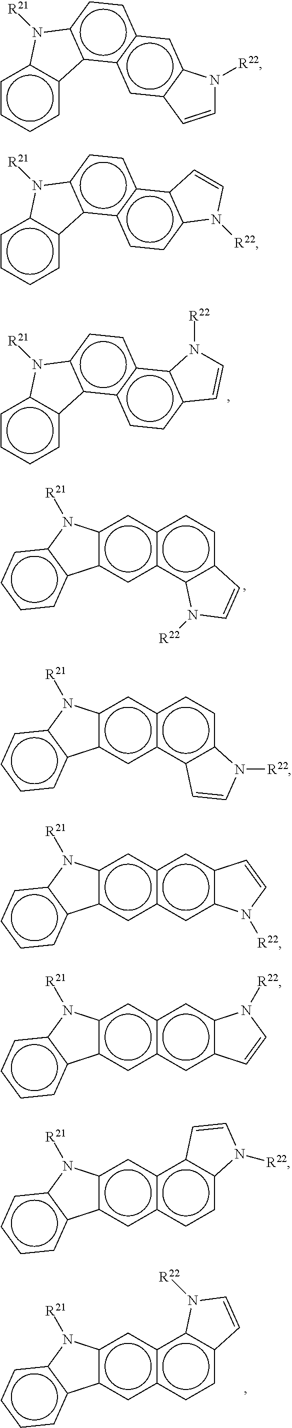

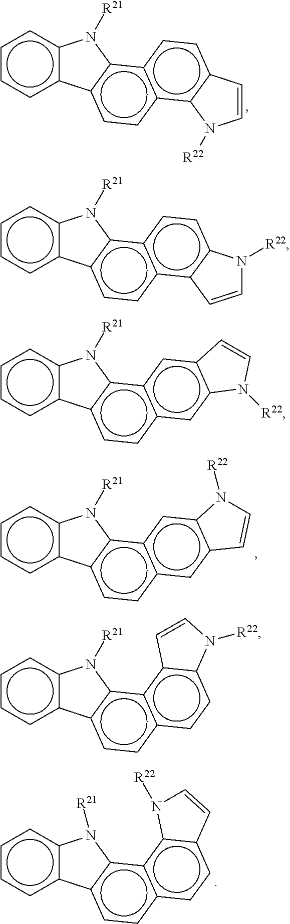

The remainder of the variables in structural formulas (VA)-(VL) are as defined above and below with respect to the nineteenth aspect.

In certain embodiments of the nineteenth aspect, R²¹ is selected from H, a C₁-C₆ alkyl, a C₃-C₁₈ cycloalkyl, a C₆-C₁₈ aryl, a 5-20 atom heteroaryl, halo, —CN, —(Ar²¹)_{d}-G, or —Ar²². According to certain embodiments, R²¹ is selected from H, a C₁-C₆ alkyl, a C₃-C₁₈ cycloalkyl, a C₆-C₁₈ aryl, a 5-20 atom heteroaryl, halo, —CN, or —(Ar²¹)_{d}—Ar²². According to certain embodiments, H, a C₁-C₆ alkyl, a C₃-C₁₈ cycloalkyl, a C₆-C₁₈ aryl, a 5-20 atom heteroaryl, halo, —CN, or —(Ar²¹)_{d}-G. According to certain embodiments, R²¹ is selected from H, a C₁-C₆ alkyl, a C₃-C₆ cycloalkyl, —(Ar²¹)_{d}-G, or —Ar²². According to certain embodiments, R²¹ is selected from H, a C₁-C₆ alkyl, a C₃-C₆ cycloalkyl, or —Ar²². According to certain embodiments, R²¹ is selected from H, a C₁-C₆ alkyl, a C₃-C₆ cycloalkyl, or —(Ar²¹)_{d}-G. The remainder of the variables in structural formulas (VA)-(VL) are as defined above and below with respect to the nineteenth aspect.

In certain embodiments of the nineteenth aspect, R²² is selected from H, a C₁-C₆ alkyl, a C₃-C₁₈ cycloalkyl, a C₆-C₁₈ aryl, a 5-20 atom heteroaryl, halo, —CN, —(Ar²¹⁻)_{d}-G, or —Ar²². According to certain embodiments, R²² is selected from H, a C₁-C₆ alkyl, a C₃-C₁₈ cycloalkyl, a C₆-C₁₈ aryl, a 5-20 atom heteroaryl, halo, —CN, or —(Ar²¹)_{d}—Ar²². According to certain embodiments, H, a C₁-C₆ alkyl, a C₃-C₁₈ cycloalkyl, a C₆-C₁₈ aryl, a 5-20 atom heteroaryl, halo, —CN, or —(Ar²¹)_{d}-G. According to certain embodiments, R²² is selected from H, a C₁-C₆ alkyl, a C₃-C₆ cycloalkyl, —(Ar²¹)_{d}-G, or —Ar²². According to certain embodiments, R²² is selected from H, a C₁-C₆ alkyl, a C₃-C₆ cycloalkyl, or —Ar²². According to certain embodiments, R²² is selected from H, a C₁-C₆ alkyl, a C₃-C₆ cycloalkyl, or —(Ar²¹)_{d}-G. The remainder of the variables in structural formulas (VA)-(VL) are as defined above and below with respect to the nineteenth aspect.

According to certain embodiments of the nineteenth aspect, at least one of R²¹ and R²² is —(Ar²¹)_{d}-G or —(Ar²¹)_{d}—Ar²². According to certain embodiments, at least one of R²¹ and R²² is —(Ar²¹)_{d}-G. According to certain embodiments, at least one of R²¹ and R²² is —(Ar²¹)_{d}-Ar²². According to certain embodiments, one of R²¹ and R²² is H or unsubstituted phenyl. According to certain embodiments, one of R²¹ and R²² is H. According to certain embodiments, one of R²¹ and R²² is unsubstituted phenyl. According to certain embodiments, R²¹ and R²² are identical.

In certain embodiments of the nineteenth aspect, Ar²¹, for each occurrence independently, is phenyl optionally substituted with one to four C₁-C₆ alkyls. In certain embodiments, Ar²¹, for each occurrence independently, is phenyl optionally substituted with one to four C₁-C₃ alkyls. In certain embodiments, Ar²¹, for each occurrence independently, is phenyl optionally substituted with one to four methyls. In certain embodiments, Ar²¹, for each occurrence, is unsubstituted phenyl. In certain embodiments, Ar²¹, for each occurrence independently, is phenyl substituted with one to four C₂-C₆ alkyls. The remainder of the variables in formulas (VA)-(VL) are as described above and below with respect to the nineteenth aspect.

In certain embodiments of the nineteenth aspect, Ar²², for each occurrence independently, is:

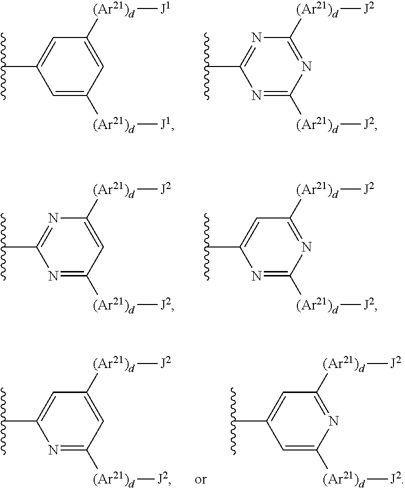

In certain embodiments, Ar²², for each occurrence independently, is:

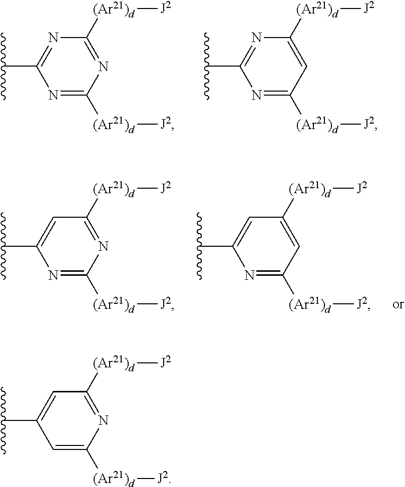

In certain embodiments, Ar²², for each occurrence independently, is:

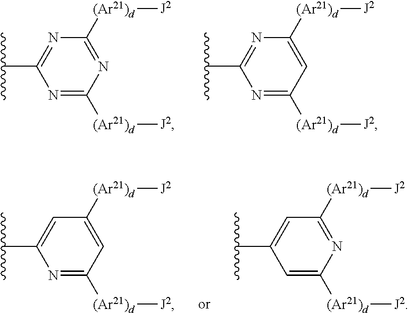

In certain embodiments, Ar²², for each occurrence, is

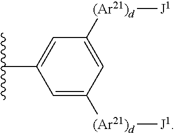

In certain embodiments, Ar²², for each occurrence, is

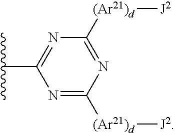

In certain embodiments, Ar²², for each occurrence, is

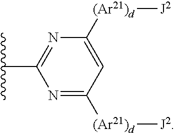

In certain embodiments, Ar²², for each occurrence, is

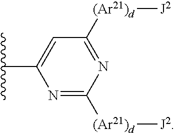

In certain embodiments, Ar²², for each occurrence, is

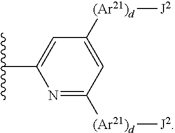

In certain embodiments, Ar²², for each occurrence, is

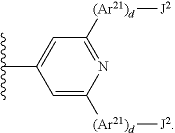

In certain embodiments, Ar²², for each occurrence independently, is optionally substituted with one to four C₁-C₆ alkyls. In certain embodiments, Ar²², for each occurrence independently, is optionally substituted with one to four C₁-C₃ alkyls. In certain embodiments, Ar²², for each occurrence independently, is optionally substituted with one to four methyls. In certain embodiments, Ar²², for each occurrence, is unsubstituted. In certain embodiments, Ar²², for each occurrence independently, is substituted with one to four C₂-C₆ alkyls. The remainder of the variables in formulas (VA)-(VL) are as described above and below with respect to the nineteenth aspect.

In certain embodiments of the nineteenth aspect, d, for each occurrence independently, is 0, 1, or 2. In certain embodiments, d, for each occurrence independently, is 0 or 1. In certain embodiments, d is 0. In certain embodiments, d is 1. In certain embodiments, d is 2. The remainder of the variables in formulas (VA)-(VL) are as described above and below with respect to the nineteenth aspect.

In certain embodiments of the nineteenth aspect, J¹, for each occurrence independently, is H, C₆-C₁₈ aryl or 5-20 atom heteroaryl. In certain embodiments of the nineteenth aspect, J¹, for each occurrence independently, is C₆-C₁₈ aryl or 5-20 atom heteroaryl. In certain embodiments of the nineteenth aspect, J¹, for each occurrence independently, is phenyl or pyridinyl. In certain embodiments of the nineteenth aspect, J¹, for each occurrence, is phenyl. In certain embodiments of the nineteenth aspect, J¹, for each occurrence, is pyridinyl. In certain embodiments, J¹ is optionally substituted with one or more —CN, —C(O)phenyl, C₁-C₆ alkyl, C₁-C₆ haloalkyl, C₆-C₁₈ aryl, or (5-6 atom) heteroaryl. In certain embodiments, J¹ is optionally substituted with —CN, C₁-C₃ alkyl, C₁-C₃ haloalkyl, C₆-C₁₀ aryl, or (5-6 atom) heteroaryl. In certain embodiments, J¹ is optionally substituted with —CN, or C₁-C₆ haloalkyl. In certain embodiments, J¹ is optionally substituted with phenyl, trifluoromethyl, or cyano. In certain embodiments, J¹ is unsubstituted. In certain embodiments, J¹ is substituted as described herein. In certain embodiments, if at least one instance of Ar²² is

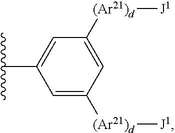

then at least one J¹ is not H or unsubstituted phenyl. The remainder of the variables in formulas (VA)-(VL) are as described above and below with respect to the nineteenth aspect.

In certain embodiments of the nineteenth aspect, J², for each occurrence independently, is H, C₆-C₁₈ aryl or 5-20 atom heteroaryl. In certain embodiments of the nineteenth aspect, J², for each occurrence independently, is C₆-C₁₈ aryl or 5-20 atom heteroaryl. In certain embodiments of the nineteenth aspect, J², for each occurrence independently, is phenyl or pyridinyl. In certain embodiments of the nineteenth aspect, J², for each occurrence, is phenyl. In certain embodiments of the nineteenth aspect, J², for each occurrence, is pyridinyl. In certain embodiments, J² is optionally substituted with one or more —CN, —C(O)phenyl, C₁-C₆ alkyl, C₁-C₆ haloalkyl, C₆-C₁₈ aryl, or (5-6 atom) heteroaryl. In certain embodiments, J² is optionally substituted with —CN, C₁-C₃ alkyl, C₁-C₃ haloalkyl, C₆-C₁₀ aryl, or (5-6 atom) heteroaryl. In certain embodiments, J² is optionally substituted with —CN, or C₁-C₆ haloalkyl. In certain embodiments, J² is optionally substituted with phenyl, trifluoromethyl, or cyano. In certain embodiments, J² is unsubstituted. In certain embodiments, J² is substituted as described herein. The remainder of the variables in formulas (VA)-(VL) are as described above and below with respect to the nineteenth aspect.

In certain embodiments of the nineteenth aspect, G, for each occurrence independently, is C₆-C₁₈ aryl or 5-20 atom heteroaryl. In certain embodiments, G is optionally substituted with one or more —CN, —C(O)phenyl, C₁-C₆ alkyl, C₁-C₆ haloalkyl, C₆-C₁₈ aryl, or (5-6 atom) heteroaryl. In certain embodiments, G is optionally substituted with —CN, C₁-C₃ alkyl, C₁-C₃ haloalkyl, C₆-C₁₀ aryl, or (5-6 atom) heteroaryl. In certain embodiments, G is optionally substituted with —CN, or C₁-C₆ haloalkyl. In certain embodiments, G is optionally substituted with phenyl, trifluoromethyl, or cyano. In certain embodiments, G is unsubstituted. In certain embodiments, G is substituted as described herein. In certain embodiments, G is not triazinyl, pyrimidinyl, tetrazolyl, oxadiazolyl, diazanaphthyl, or pentafluorophenyl. In certain embodiments, if G is phenyl it is not unsubstituted and is not substituted with carbonyl, trifluoromethyl, triazinyl, pyrimidinyl, tetrazolyl, oxadiazolyl, diazanaphthyl, or pentafluorophenyl. In certain embodiments, G is phenyl substituted with up to 5 C₁-C₆ haloalkyls. In certain embodiments, G is phenyl substituted with up to 5 trifluoromethyls. The remainder of the variables in formulas (VA)-(VL) are as described above and below with respect to the nineteenth aspect.

In certain embodiments of the nineteenth aspect, E²³ is CR^{Y} or N. In certain embodiments, E²³ is N. In certain embodiments, E²³ is CR^{Y}. The remainder of the variables in formulas (VA)-(VL) are as described above and below with respect to the nineteenth aspect.

In certain embodiments of the nineteenth aspect, E²⁴ is CR^{Y} or N. In certain embodiments, E²⁴ is N. In certain embodiments, E²⁴ is CR^{Y}. The remainder of the variables in formulas (VA)-(VL) are as described above and below with respect to the nineteenth aspect.

In certain embodiments of the nineteenth aspect, E²⁵ is CR^{Y} or N. In certain embodiments, E²⁵ is N. In certain embodiments, E²⁵ is CR^{Y}. The remainder of the variables in formulas (VA)-(VL) are as described above and below with respect to the nineteenth aspect.

In certain embodiments of the nineteenth aspect, E²⁶ is CR^{Y} or N. In certain embodiments, E²⁶ is N. In certain embodiments, E²⁶ is CR^{Y}. The remainder of the variables in formulas (VA)-(VL) are as described above and below with respect to the nineteenth aspect.

In certain embodiments of the nineteenth aspect, R^{Y}, for each occurrence independently, is H, a C₁-C₆ alkyl, a C₃-C₁₈ cycloalkyl, a C₆-C₁₈ aryl, a 5-20 atom heteroaryl, halo, or —CN. In certain embodiments, R^{Y}, for each occurrence independently, is H or a C₁-C₆ alkyl. The remainder of the variables in formulas (VA)-(VL) are as described above and below with respect to the nineteenth aspect.

In certain embodiments of the nineteenth aspect, R²³¹ is H, a C₁-C₆ alkyl, a C₃-C₁₈ cycloalkyl, a C₆-C₁₈ aryl, a 5-20 atom heteroaryl, halo, or —CN. In certain embodiments, R²³¹ is H, a C₁-C₆ alkyl, or a C₃-C₆ cycloalkyl. In certain embodiments, R²³¹ is H. In certain embodiments, R²³¹ is a C₁-C₆ alkyl, phenyl, or a C₃-C₆ cycloalkyl. The remainder of the variables in formulas (VA)-(VL) are as described above and below with respect to the nineteenth aspect.

In certain embodiments of the nineteenth aspect, R²³² is H, a C₁-C₆ alkyl, a C₃-C₁₈ cycloalkyl, a C₆-C₁₈ aryl, a 5-20 atom heteroaryl, halo, or —CN. In certain embodiments, R²³² is H, a C₁-C₆ alkyl, or a C₃-C₆ cycloalkyl. In certain embodiments, R²³² is H. In certain embodiments, R²³² is a C₁-C₆ alkyl, phenyl, or a C₃-C₆ cycloalkyl. The remainder of the variables in formulas (VA)-(VL) are as described above and below with respect to the nineteenth aspect.

In certain embodiments of the nineteenth aspect, R²⁴¹ is H, a C₁-C₆ alkyl, a C₃-C₁₈ cycloalkyl, a C₆-C₁₈ aryl, a 5-20 atom heteroaryl, halo, or —CN. In certain embodiments, R²⁴¹ is H, a C₁-C₆ alkyl, or a C₃-C₆ cycloalkyl. In certain embodiments, R²⁴¹ is H. In certain embodiments, R²⁴¹ is a C₁-C₆ alkyl, phenyl, or a C₃-C₆ cycloalkyl. The remainder of the variables in formulas (VA)-(VL) are as described above and below with respect to the nineteenth aspect.

In certain embodiments of the nineteenth aspect, R²⁴² is H, a C₁-C₆ alkyl, a C₃-C₁₈ cycloalkyl, a C₆-C₁₈ aryl, a 5-20 atom heteroaryl, halo, or —CN. In certain embodiments, R²⁴² is H, a C₁-C₆ alkyl, or a C₃-C₆ cycloalkyl. In certain embodiments, R²⁴² is H. In certain embodiments, R²⁴² is a C₁-C₆ alkyl, phenyl, or a C₃-C₆ cycloalkyl. The remainder of the variables in formulas (VA)-(VL) are as described above and below with respect to the nineteenth aspect.

In certain embodiments of the nineteenth aspect, R²⁵¹ is H, a C₁-C₆ alkyl, a C₃-C₁₈ cycloalkyl, a C₆-C₁₈ aryl, a 5-20 atom heteroaryl, halo, or —CN. In certain embodiments, R²⁵¹ is H, a C₁-C₆ alkyl, or a C₃-C₆ cycloalkyl. In certain embodiments, R²⁵¹ is H. In certain embodiments, R²⁵¹ is a C₁-C₆ alkyl, phenyl, or a C₃-C₆ cycloalkyl. The remainder of the variables in formulas (VA)-(VL) are as described above and below with respect to the nineteenth aspect.

In certain embodiments of the nineteenth aspect, R²⁵² is H, a C₁-C₆ alkyl, a C₃-C₁₈ cycloalkyl, a C₆-C₁₈ aryl, a 5-20 atom heteroaryl, halo, or —CN. In certain embodiments, R²⁵² is H, a C₁-C₆ alkyl, or a C₃-C₆ cycloalkyl. In certain embodiments, R²⁵² is H. In certain embodiments, R²⁵² is a C₁-C₆ alkyl, phenyl, or a C₃-C₆ cycloalkyl. The remainder of the variables in formulas (VA)-(VL) are as described above and below with respect to the nineteenth aspect.

In certain embodiments of the nineteenth aspect,

E²³, E²⁴, E²⁵, and E²⁶ are, each independently, CR^{Y} or N, wherein R^{Y}, for each occurrence independently, is H or a C₁-C₆ alkyl;

R²³¹, R²³², R²⁴¹, R²⁴², R²⁵¹, and R²⁵² are, each independently, H, a C₁-C₆ alkyl, or a C₃-C₆ cycloalkyl;

R²¹ and R²², each independently, are selected from H, a C₁-C₆ alkyl, a C₃-C₆ cycloalkyl, —(Ar²¹)_{d}-G, or —Ar²²;

Ar²¹, for each occurrence independently, is phenyl optionally substituted with one to four C₁-C₃ alkyls;

Ar²², for each occurrence independently, is

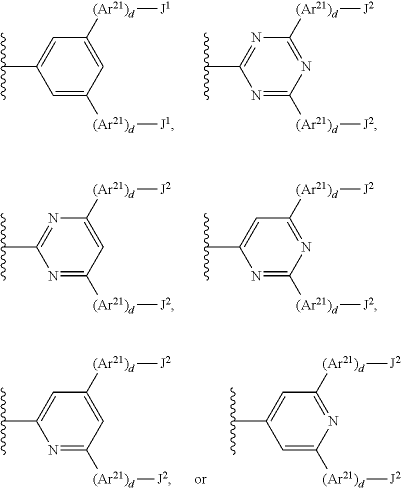

and is optionally substituted with one to three C₁-C₃ alkyls;

d, for each occurrence independently, is 0, 1, or 2;

G, for each occurrence independently, is phenyl substituted with 1, 2, 3, 4, or 5 trifluoromethyls; and

each occurrence of J¹ or J², is independently, phenyl or pyridinyl, and is optionally substituted with 1, 2, 3, 4, or 5 substituents selected from phenyl, trifluoromethyl, or cyano.

In certain embodiments of the nineteenth aspect, the molecule is represented by one of the following structural formulas:

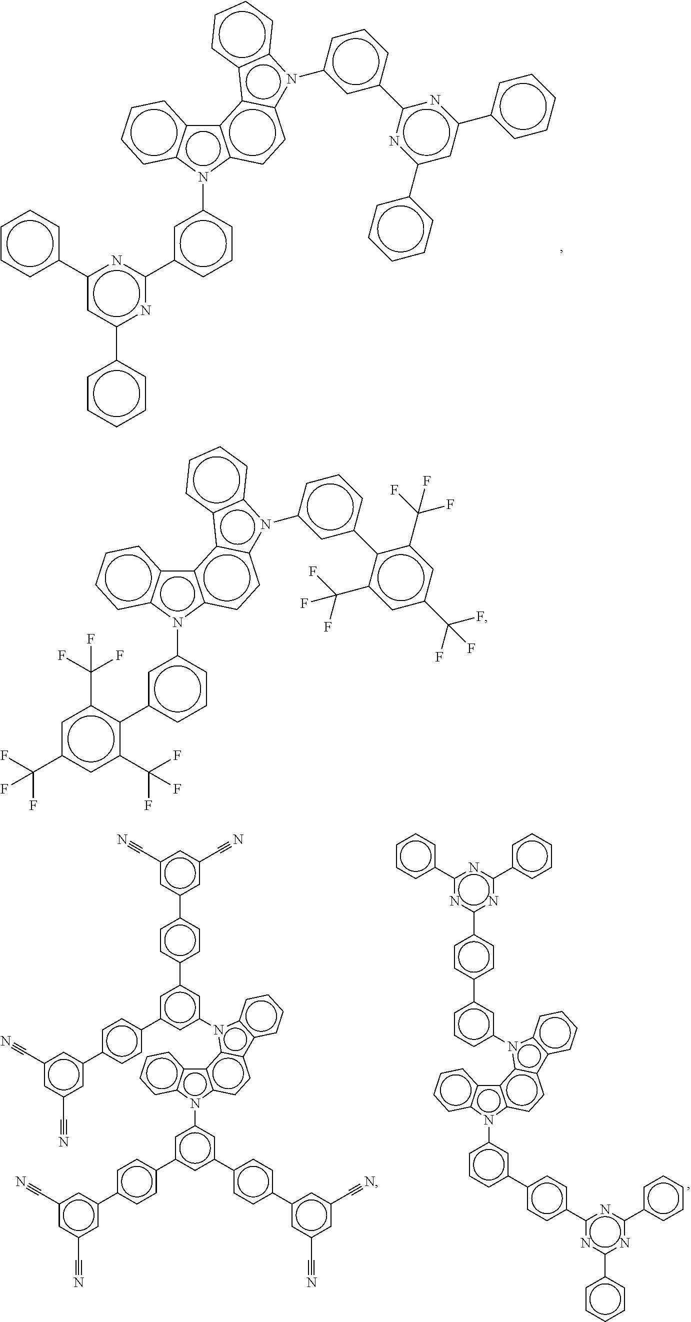

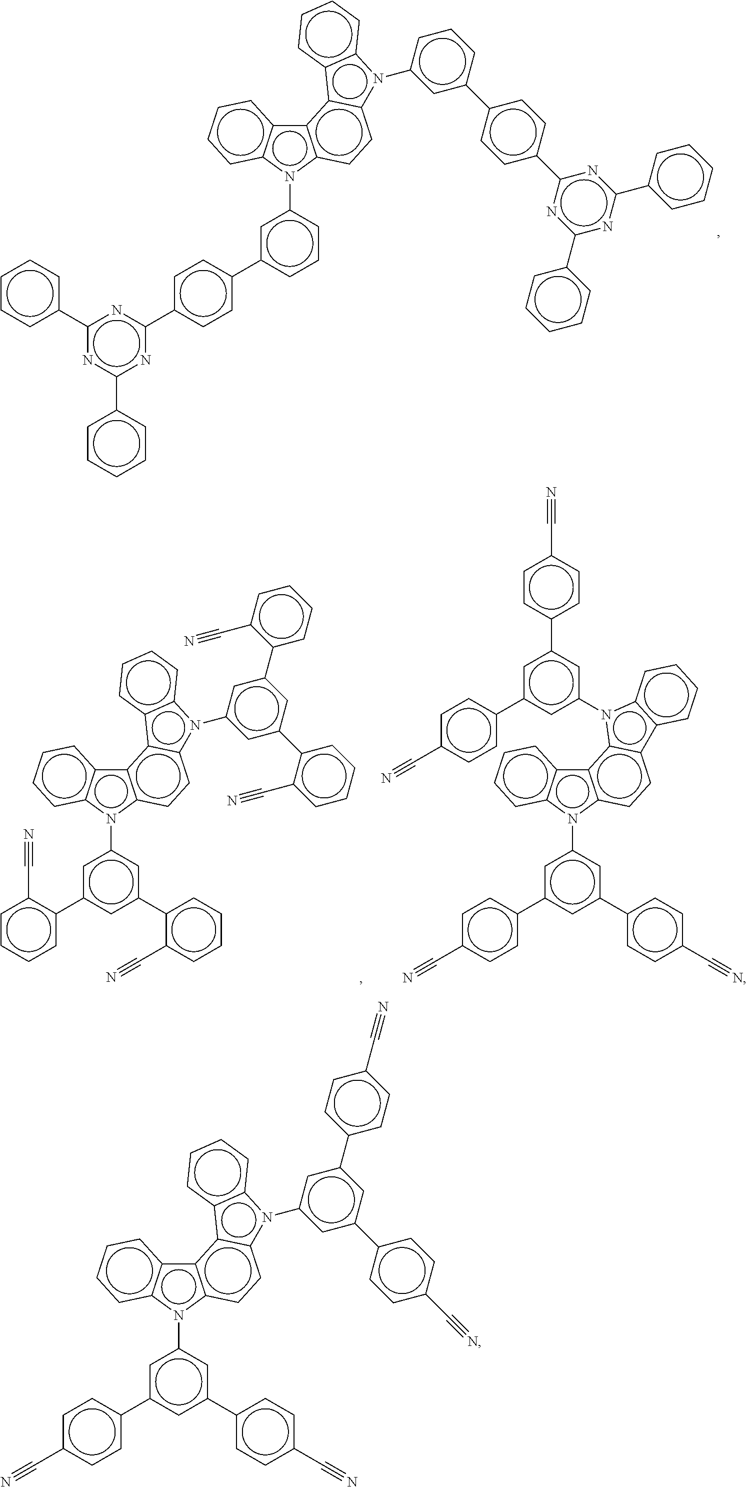

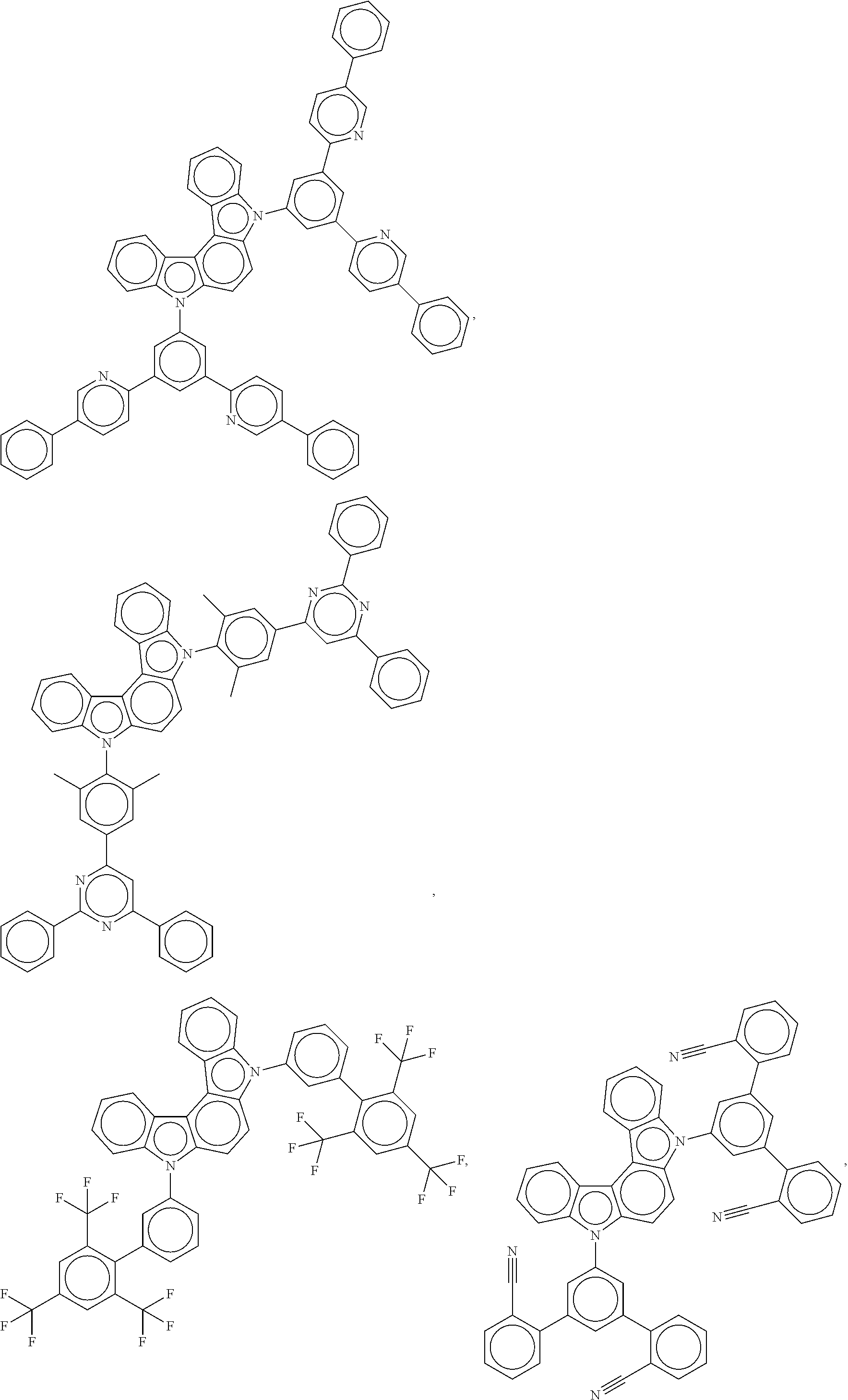

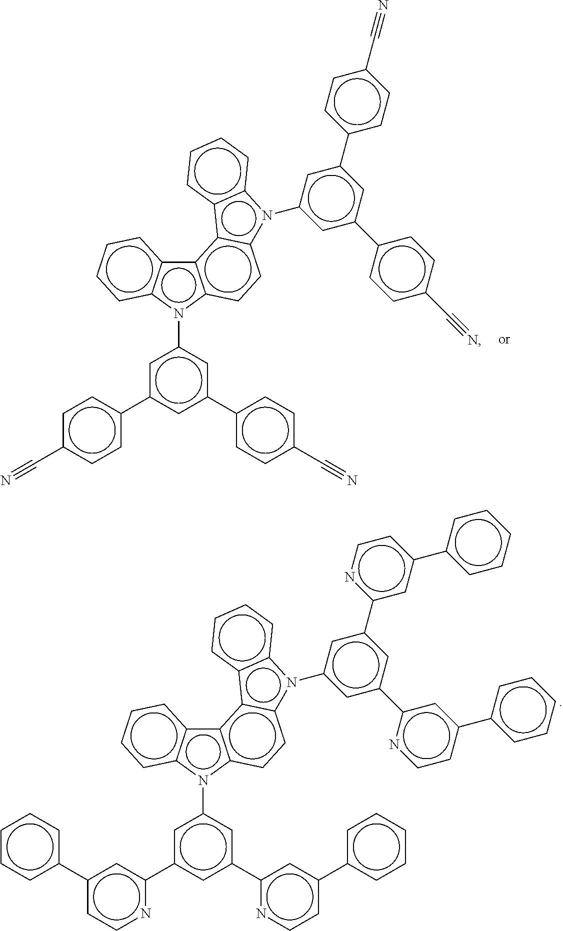

TABLE NR

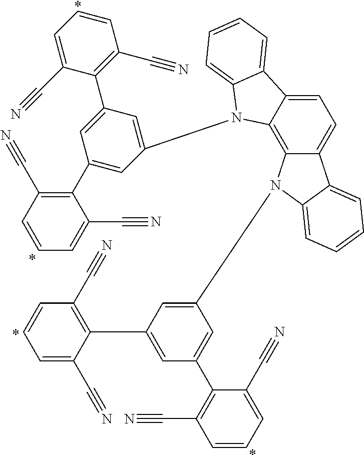

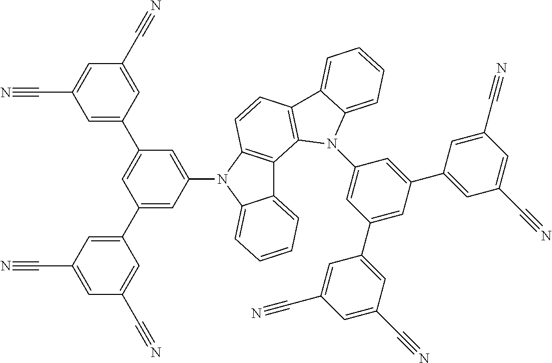 [SMILES: N#Cc1cc(C#N)cc(-c2cc(-c3cc(C#N)cc(C#N)c3)cc(-n3c4ccccc4c4c3ccc3c5ccccc5n(-c5cc(-c6cc(C#N)cc(C#N)c6)cc(-c6cc(C#N)cc(C#N)c6)c5)c34)c2)c1]

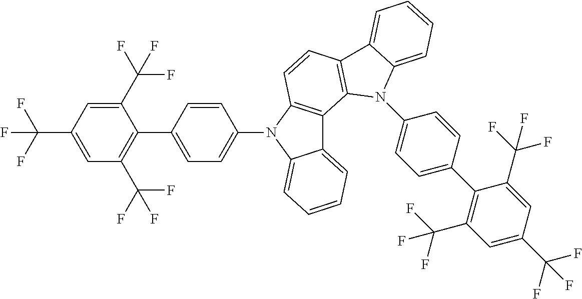 [SMILES: FC(F)(F)c1cc(C(F)(F)F)c(-c2ccc(-n3c4ccccc4c4c3ccc3c5ccccc5n(-c5ccc(-c6c(C(F)(F)F)cc(C(F)(F)F)cc6C(F)(F)F)cc5)c34)cc2)c(C(F)(F)F)c1]

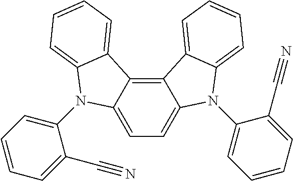 [SMILES: N#Cc1ccccc1-n1c2ccccc2c2c3c4ccccc4n(-c4ccccc4C#N)c3ccc21]

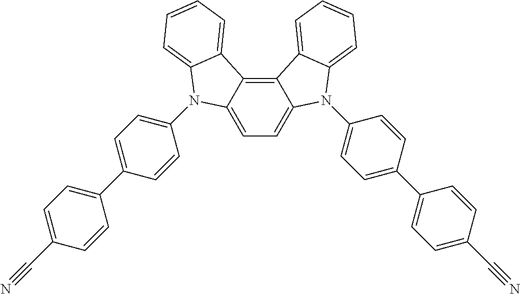 [SMILES: N#Cc1ccc(-c2ccc(-n3c4ccccc4c4c5c6ccccc6n(-c6ccc(-c7ccc(C#N)cc7)cc6)c5ccc43)cc2)cc1]

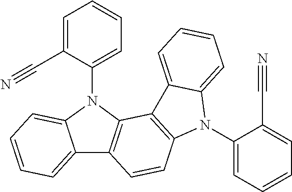 [SMILES: N#Cc1ccccc1-n1c2ccccc2c2c1ccc1c3ccccc3n(-c3ccccc3C#N)c12]

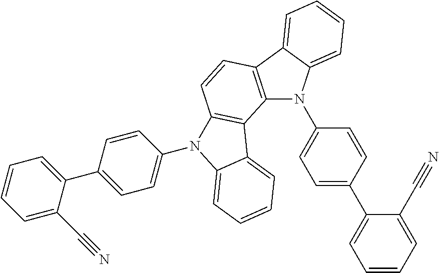 [SMILES: N#Cc1ccccc1-c1ccc(-n2c3ccccc3c3c2ccc2c4ccccc4n(-c4ccc(-c5ccccc5C#N)cc4)c23)cc1]

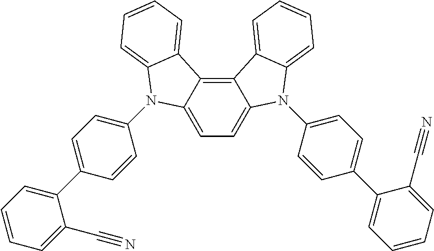 [SMILES: N#Cc1ccccc1-c1ccc(-n2c3ccccc3c3c4c5ccccc5n(-c5ccc(-c6ccccc6C#N)cc5)c4ccc32)cc1]

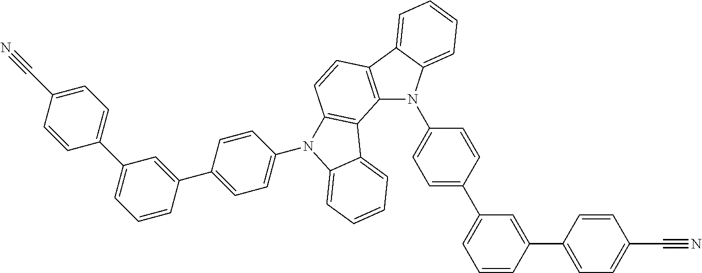 [SMILES: N#Cc1ccc(-c2cccc(-c3ccc(-n4c5ccccc5c5c4ccc4c6ccccc6n(-c6ccc(-c7cccc(-c8ccc(C#N)cc8)c7)cc6)c45)cc3)c2)cc1]

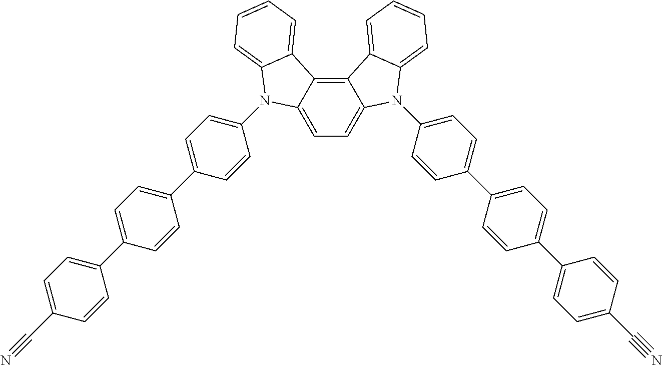 [SMILES: N#Cc1ccc(-c2ccc(-c3ccc(-n4c5ccccc5c5c6c7ccccc7n(-c7ccc(-c8ccc(-c9ccc(CN)cc9)cc8)cc7)c6ccc54)cc3)cc2)cc1]

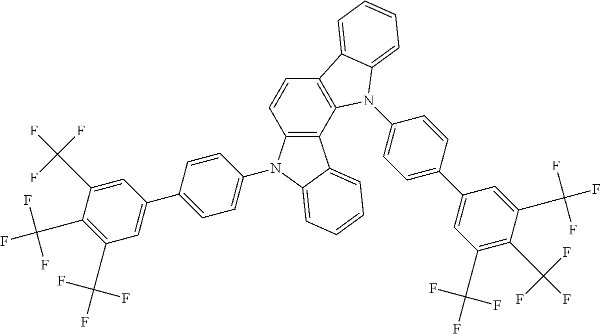 [SMILES: FC(F)(F)c1cc(-c2ccc(-n3c4ccccc4c4c3ccc3c5ccccc5n(-c5ccc(-c6cc(C(F)(F)F)c(C(F)(F)F)c(C(F)(F)F)c6)cc5)c34)cc2)cc(C(F)(F)F)c1C(F)(F)F]

In a twentieth aspect, the present invention is a molecule represented by structural formula (VI):

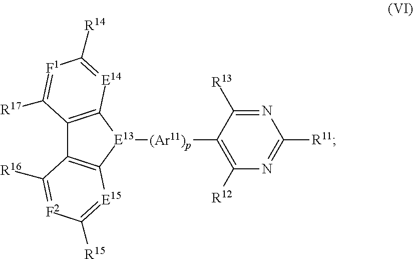

In structural formula (VI) of the present invention:

E¹³ E¹⁴, and E¹⁵ are, each independently, CR^{A} or N.

R^{A}, for each occurrence independently, is H, a C₁-C₆ alkyl, a C₃-C₁₈ cycloalkyl, a C₆-C₁₈ aryl, a 5-20 atom heteroaryl, a halo, or —CN.

At least one of E¹³, E¹⁴, and E¹⁵ is N.

R¹¹, R¹², and R¹³ are, each independently, H, a C₁-C₆ alkyl, a C₃-C₁₈ cycloalkyl, a C₆-C₁₈ aryl, a 5-20 atom heteroaryl, a halo, or —CN.

R¹⁴, R¹⁵, R¹⁶, and R¹⁷ are, each independently, H, a C₁-C₆ alkyl, a C₃-C₁₈ cycloalkyl, a C₆-C₁₈ aryl, a 5-20 atom heteroaryl, a halo, or —CN.

F^{l} is C—(Ar¹²)_{q}-G.

F² is CR^{B} or N, wherein R^{B} is H, a C₁-C₆ alkyl, a C₃-C₆ cycloalkyl, a C₆-C₁₈aryl, a 5-20 atom heteroaryl, a halo, or —CN, or —(Ar¹²)_{q}-G.

Ar¹¹, for each occurrence independently, is phenyl optionally substituted with one to four C₁-C₆ alkyls.

Ar¹², for each occurrence independently, is phenyl optionally substituted with one to four C₁-C₆ alkyls.

p is 0, 1, or 2.

q is 0 or 1.

G, for each occurrence independently, is a moiety represented by one of the following structural formulas:

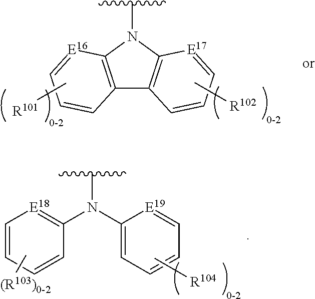

E¹⁶, E¹⁷, E¹⁸, and E¹⁹ are, each independently, CR^{C} or N, wherein R^{C} is H, a C₁-C₃ alkyl, halo, or —CN.

R¹⁰¹, R¹⁰², R¹⁰³, and R¹⁰⁴ are, each independently, a C₁-C₆ alkyl, a C₃-C₁₈ cycloalkyl, a C₆-C₁₈ aryl, a 5-20 atom heteroaryl, a halo, or —CN.

In one example embodiment of the twentieth aspect, the molecule of structural formula (VI) is not represented by any structural formula in Table 11. In a further example embodiment, in Table 11, each carbon or heteroatom denoted by * in the structural formulas therein is unsubstituted or substituted by a C₁-C₆ alkyl, —OH, —CN, a halo, a C₆-C₁₂ aryl, a 5-20 atom heteroaryl, —N(R³⁰⁰)₂, or —N(R³⁰¹)₂, wherein each R³⁰⁰, independently, is H or a C₁-C₆ alkyl and wherein each R³⁰¹, independently, is H or a C₆-C₁₈ aryl.

In another example embodiment of the twentieth aspect:

R¹¹ is a C₁-C₆ alkyl, a C₃-C₁₈ cycloalkyl, a C₆-C₁₈ aryl, a 5-20 atom heteroaryl, a halo, or —CN.

Ar¹¹, for each occurrence independently, is phenyl optionally substituted with one to four C₂-C₆ alkyls.

p is 0 or 1.

Provided that if p is 1, Ar¹¹ is unsubstituted phenyl, and R¹¹ is unsubstituted phenyl, then F² is not CH.

In an example embodiment of the twentieth aspect:

R¹¹ is C₁-C₆ alkyl or C₆-C₁₈ aryl

R¹² and R¹³ are, each independently, H, C₁-C₆ alkyl, or C₆-C₁₈ aryl.

In an example embodiment of the twentieth aspect:

R¹¹ is a C₆-C₁₈ aryl and R¹² and R¹³ are, each independently, H or a C₁-C₆ alkyl.

In an example embodiment of the twentieth aspect:

Ar¹¹ is a phenyl.

In an example embodiment of the twentieth aspect:

F² is CR^{B}.

In an example embodiment of the twentieth aspect:

G, for each occurrence independently, is a moiety represented by one of the following structural formulas:

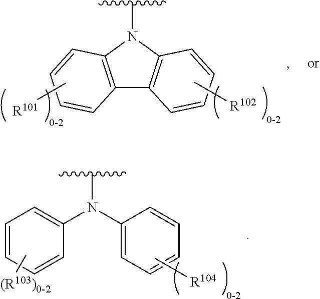

In an example embodiment of the twentieth aspect:

R^{A}, for each occurrence independently, is H or a C₁-C₆ alkyl.

R¹¹ is a C₆-C₁₈ aryl, and R¹² and R¹³ are, each independently, H or a C₁-C₃ alkyl.

R14_{, R}¹⁵, R¹⁶, and R¹⁷ are, each independently, H, a C₁-C₆ alkyl, or a C₃-C₆ cycloalkyl.

F₁ and F₂ are CR^{B} wherein R^{B} is, for each occurrence independently, H, a C₁-C₆ alkyl, a C₃-C₆ cycloalkyl, or —(Ar¹²)_{q}-G.

In an example embodiment of the twentieth aspect, structural formula (I) can be represented by the following structural formula:

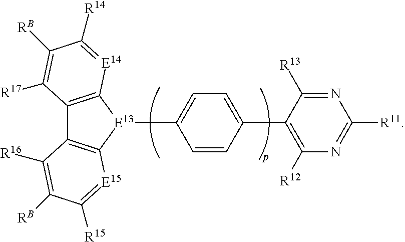

E¹³, E¹⁴, and E¹⁵, are, each independently, CR^{A} or N, wherein R^{A}, for each occurrence independently, is H or a C₁-C₆ alkyl.

p is 0 or 1.

R¹¹ is a C₆-C₁₈ aryl or a 5-20 atom heteroaryl.

R¹² and R¹³ are, each independently, H, a C₁-C₆ alkyl, a halo, or —CN.

R¹⁴, R¹⁵, R¹⁶, and R¹⁷ are, each independently, H, a C₁-C₆ alkyl, a C₃-C₁₈ cycloalkyl, a halo, or —CN.

R^{B} is, for each occurrence independently, H, a C₁-C₆ alkyl, a C₃-C₆ cycloalkyl, or a moiety represented by one of the following structural formulas:

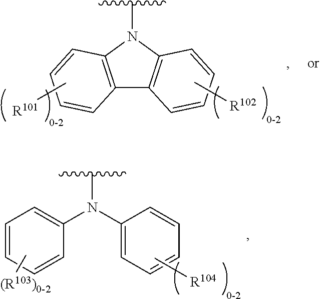

R¹⁰¹, R¹⁰², R¹⁰³, and R¹⁰⁴ are, each independently, a C₁-C₆ alkyl or a C₃-C₆ cycloalkyl.

In an example embodiment of the twentieth aspect:

E¹³ is N; and

E¹⁴ and E¹⁵ are CR^{A}.

In an example embodiment of the twentieth aspect, R^{B} is, for each occurrence independently, H or a moiety represented by the following structural formula:

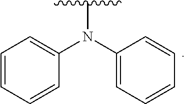 [SMILES: *N(c1ccccc1)c1ccccc1]

In an example embodiment of the twentieth aspect:

R¹¹ is a C₆-C₁₈ aryl.

R¹² and R¹³ are, each independently, H or a C₁-C₃ alkyl.

R¹⁴, R¹⁵, R¹⁶, and R¹⁷ are, each independently, H, a C₁-C₆ alkyl, or a C₃-C₁₈ cycloalkyl.

In an example embodiment of the twentieth aspect, molecular structure (VI) can be represented by the following structural formula:

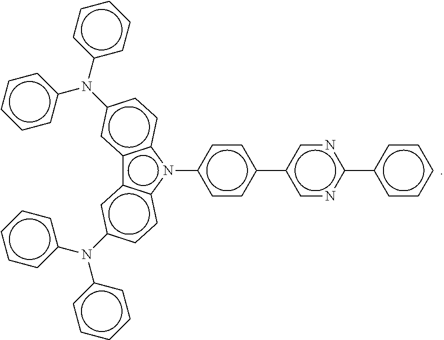

In an example embodiment of the twentieth aspect, molecular structure (VI) can be represented by the following structural formula:

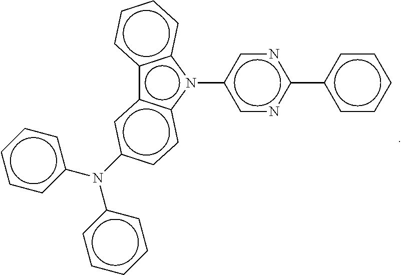

In a twenty-first aspect, the present invention is a molecule that can be represented by one of structural formulas (VIIA), (VIM), or (VIIC):

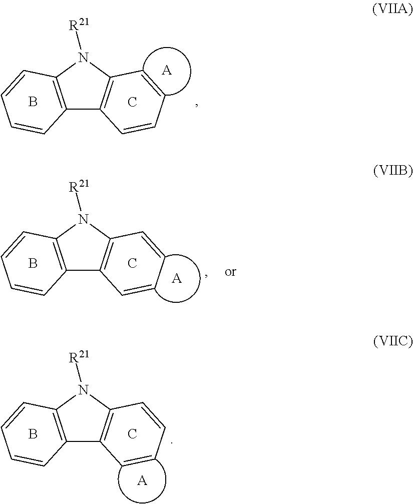

In structural formulas (VIIA), (VIIB), and (VIIC):

Ring A, for each occurrence independently, is represented by the following structural formula:

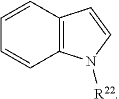

Ring A may be fused to ring C in any orientation. In certain embodiments, any two atoms of the hexacycle of ring A may be shared with ring C. In other embodiments, the two carbon atoms of the pentacycle of ring A may be shared with ring C. In example embodiments, the compounds of structures (VIIA), (VIIB), and (VIIC) can be represented by the following structural formulas:

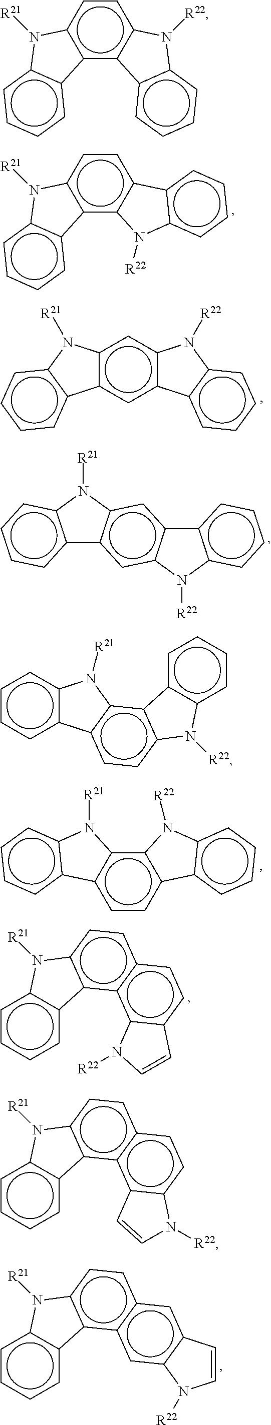

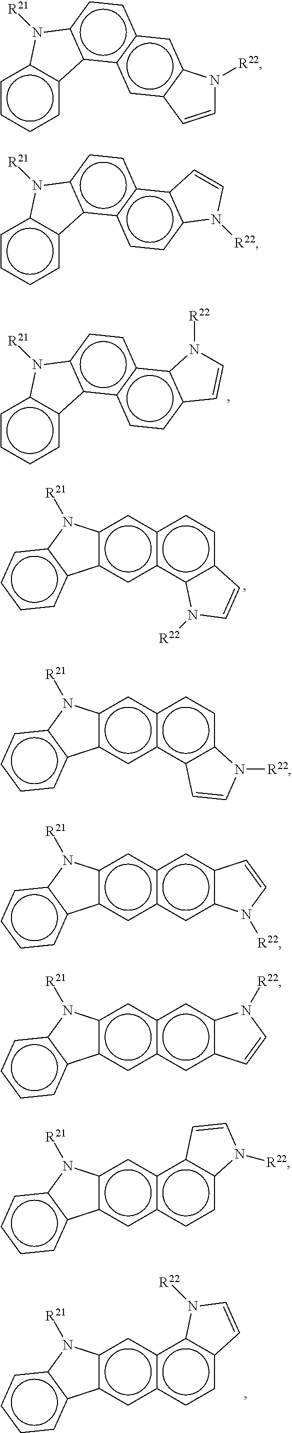

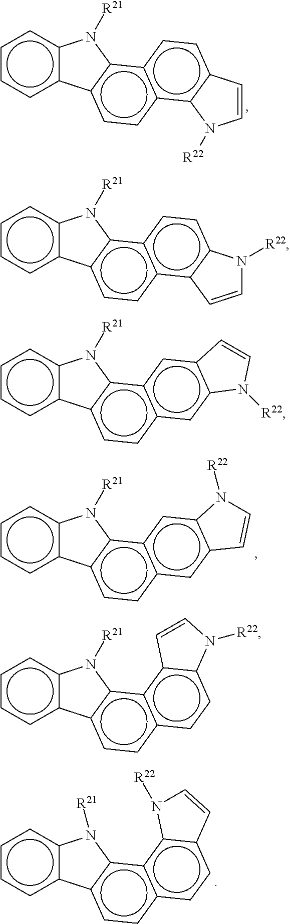

Rings A, B, and C, each independently, are optionally substituted with 1 to 4 substituents selected from a C₁-C₆ alkyl, a C₃-C₁₈ cycloalkyl, a C₆-C₁₈ aryl, a 5-20 atom heteroaryl, halo, or —CN.

R²¹ and R²², each independently, are selected from H, a C₁-C₆ alkyl, a C₃-C₁₈ cycloalkyl, a C₆-C₁₈ aryl, a 5-20 atom heteroaryl, halo, —CN, or —(Ar²¹)_{d}-G. At least one of R²¹ and R²² is —(Ar²¹)_{d}-G.

Ar²¹, for each occurrence independently, is phenyl optionally substituted with one to four C₁-C₆ alkyls.

d, for each occurrence independently, is 0, 1, or 2.

G, for each occurrence independently, is:

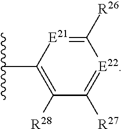

E²¹ and E²² are, each independently, CR^{X} or N, wherein R^{X}, for each occurrence independently, is H, a C₁-C₆ alkyl, a C₃-C₁₈ cycloalkyl, a C₆-C₁₈ aryl, a 5-20 atom heteroaryl, a halo, or —CN. At least one of E²¹ and E²² is N.

R²⁶, R²⁷, and R²⁸, for each occurrence independently, are H, a C₁-C₆ alkyl, a C₃-C₁₈ cycloalkyl, a C₆-C₁₈ aryl, a 5-20 atom heteroaryl, a halo, or —CN.

In an example embodiment of the twenty-first aspect, the molecule is represented by structural formula (VIID):

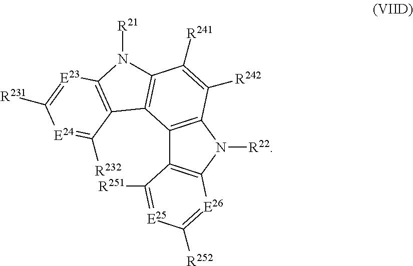

In structural formula (III):

E²³, E²⁴, E²⁵, and E²⁶ are, each independently, CR^{Y} or N, wherein R^{Y}, for each occurrence independently, is H, a C₁-C₆ alkyl, a C₃-C₁₈ cycloalkyl, a C₆-C₁₈ aryl, a 5-20 atom heteroaryl, halo, or —CN.

R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN.

In an example embodiment of the twenty-first aspect:

R²⁶, R²⁷, and R²⁸ are, each independently, H, C₁-C₆ alkyl, or C₆-C₁₈ aryl.

d is 1 or 2.

In an example embodiment of the twenty-first aspect:

R²⁶ and R²⁷, each independently, are a C₆-C₁₈ aryl, and R²⁸ is H or a C₁-C₆ alkyl.

In an example embodiment of the twenty-first aspect:

Ar²¹ is a moiety represented by the following structural formula:

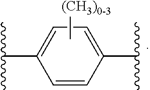

In an example embodiment of the twenty-first aspect:

E²³, E²⁴, E²⁵, and E²⁶ are, each independently, CR^{Y} or N, wherein R^{Y}, for each occurrence independently, is H or a C₁-C₆ alkyl;

R²⁶ and R²⁷, each independently, are a C₆-C₁₈ aryl, and R²⁸ is H or a C₁-C₃ alkyl; and

R²³¹, R²³², R²⁴¹, R²⁴², R²⁵¹, and R²⁵² are, each independently, H, a C₁-C₆ alkyl, or a C₃-C₆ cycloalkyl;

R²¹ and R²², each independently, are selected from H, a C₁-C₆ alkyl, a C₃-C₆ cycloalkyl, or —(Ar²¹)_{d}-G;

Ar²¹, for each occurrence independently, is phenyl optionally substituted with one to four C₁-C₃ alkyls;

d, for each occurrence independently, is 0, 1, or 2;

G, for each occurrence independently, is:

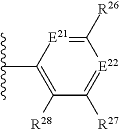

E²¹ and E²² are, each independently, CR^{X} or N, wherein R^{X}, for each occurrence independently, is H, or a C₁-C₆ alkyl.

In an example embodiment of the twenty-first aspect:

E²¹ and E²² are N; and

E²³, E²⁴, E²⁵, and E²⁶ are, each independently, CR^{Y}.

In an example embodiment of the twenty-first aspect:

R²¹ and R²² are, each independently, selected from H, C₁-C₆ alkyl, C₆-C₁₈ aryl, 5-20 atom heteroaryl, or a moiety represented by the following structural formula:

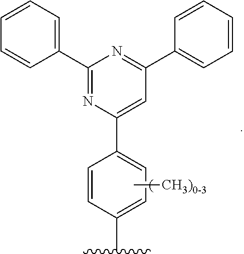

In an example embodiment of the twenty-first aspect:

R¹¹ is a C₆-C₁₈ aryl.

R¹² and R¹³ are, each independently, H or a C₁-C₃ alkyl.

R¹⁴, R¹⁵, R¹⁶, and R¹⁷ are, each independently, H, a C₁-C₆ alkyl, or a C₃-C₁₈ cycloalkyl.

In an example embodiment of the twenty-first aspect, the molecule is represented by the structure:

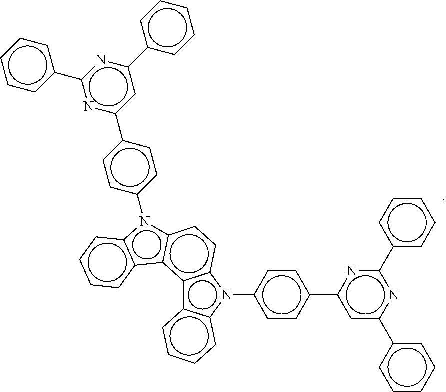

In another example embodiment of the twenty-first aspect, the molecule is represented by the structure:

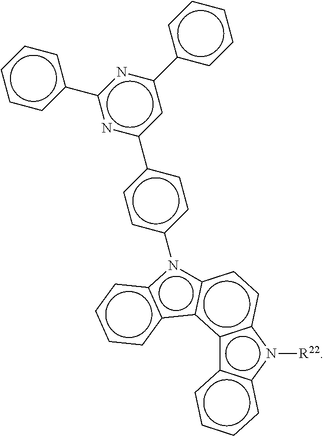

In this structure, R²² is H or C₁-C₆ alkyl.

In an example embodiment of the twenty-first aspect, the molecule is represented by structural formula (VIIE):

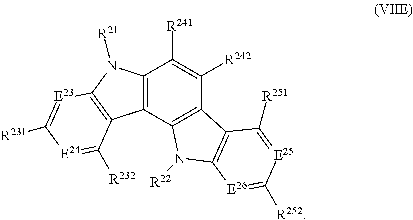

In structural formula (VIIE):

E²³, E²⁴, E²⁵, and E²⁶ are, each independently, CR^{Y} or N, wherein R^{Y}, for each occurrence independently, is H, a C₁-C₆ alkyl, a C₃-C₁₈ cycloalkyl, a C₆-C₁₈ aryl, a 5-20 atom heteroaryl, halo, or —CN.

R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN.

In an example embodiment of the twenty-first aspect:

R²⁶, R²⁷, and R²⁸ are, each independently, H, C₁-C₆ alkyl, or C₆-C₁₈ aryl.

d is 1 or 2.

In an example embodiment of the twenty-first aspect:

R²⁶ and R²⁷, each independently, are a C₆-C₁₈ aryl, and R²⁸ is H or a C₁-C₆ alkyl.

In an example embodiment of the twenty-first aspect:

Ar²¹ is a moiety represented by the following structural formula:

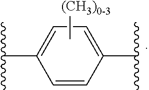

In an example embodiment of the twenty-first aspect:

E²³, E²⁴, E²⁵, and E²⁶ are, each independently, CR^{Y} or N, wherein R^{Y}, for each occurrence independently, is H or a C₁-C₆ alkyl;

R²⁶ and R²⁷, each independently, are a C₆-C₁₈ aryl, and R²⁸ is H or a C₁-C₃ alkyl; and

R231, R²³², R²⁴¹, R²⁴², R²⁵¹, and R²⁵² are, each independently, H, a C₁-C₆ alkyl, or a C₃-C₆ cycloalkyl;

R²¹ and R²², each independently, are selected from H, a C₁-C₆ alkyl, a C₃-C₆ cycloalkyl, or —(Ar²¹)_{d}-G;

Ar²¹, for each occurrence independently, is phenyl optionally substituted with one to four C₁-C₃ alkyls;

d, for each occurrence independently, is 0, 1, or 2;

G, for each occurrence independently, is:

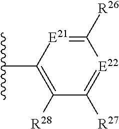

E²¹ and E²² are, each independently, CR^{X} or N, wherein R^{X}, for each occurrence independently, is H, or a C₁-C₆ alkyl.

In an example embodiment of the twenty-first aspect:

E²¹ and E²² are N; and

E²³, E²⁴, E²⁵, and E²⁶ are, each independently, CR^{Y}.

In an example embodiment of the twenty-first aspect:

R²¹ and R²² are, each independently, selected from H, C₁-C₆ alkyl, C₆-C₁₈ aryl, 5-20 atom heteroaryl, or a moiety represented by the following structural formula:

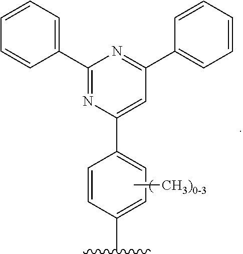

In an example embodiment of the twenty-first aspect:

R¹¹ is a C₆-C₁₈ aryl.

R¹² and R¹³ are, each independently, H or a C₁-C₃ alkyl.

R¹⁴, R¹⁵, R¹⁶, and R¹⁷ are, each independently, H, a C₁-C₆ alkyl, or a C₃-C₁₈ cycloalkyl.

In an example embodiment of the twenty-first aspect, the molecule is represented by the following structure:

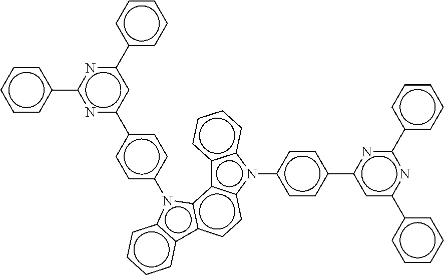

In another example embodiment of the twenty-first aspect, the molecule is represented by the following structure:

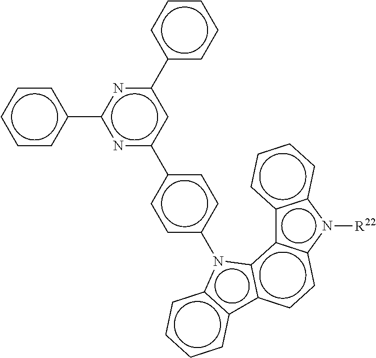

In this structure, R²² is H or C₁-C₆ alkyl.

In a twenty-second aspect, the present invention is a molecule represented by any one of the following structural formulas:

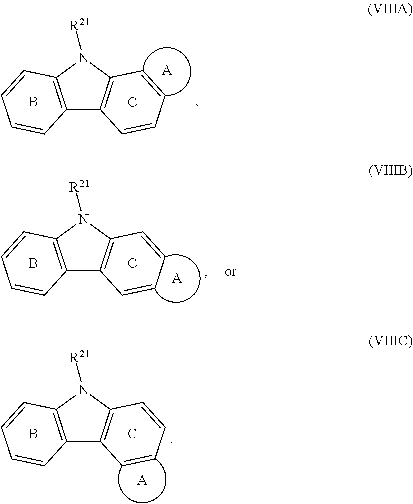

In structural formulas (VIIIA), (VIIIB), and (VIIIC) of the present invention: Ring A, for each occurrence independently, is represented by the following structural formula:

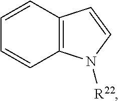

Ring A may be fused to ring C in any orientation. In certain embodiments, any two atoms of the hexacycle of ring A may be shared with ring C. In other embodiments, the two carbon atoms of the pentacycle of ring A may be shared with ring C. In example embodiments, the compounds of structures (VIIIA), (VIIIB), and (VIIIC) can be represented by the following structural formulas:

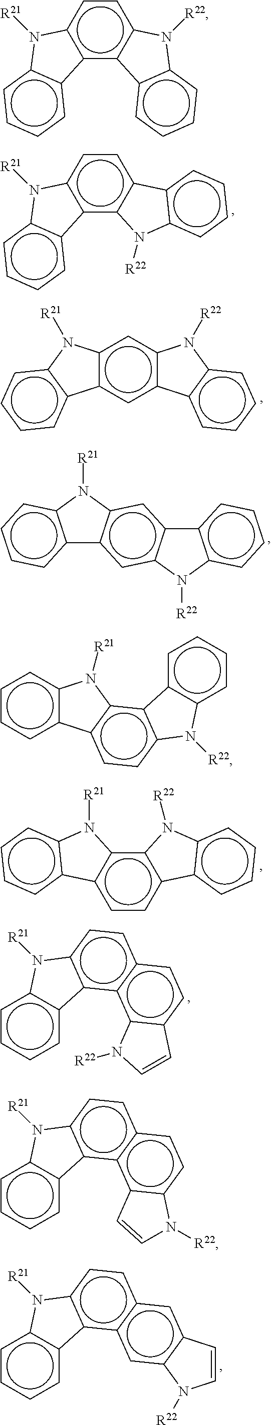

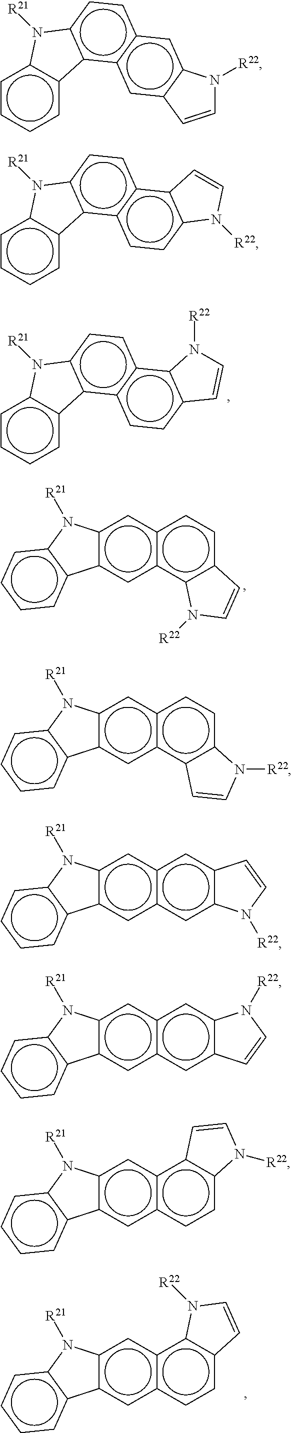

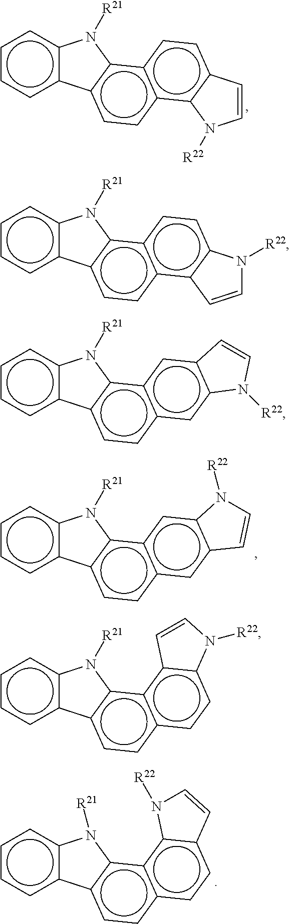

Rings A, B, and C, each independently, are optionally substituted with 1 to 4 substituents selected from a C₁-C₆ alkyl, a C₃-C₁₈ cycloalkyl, a C₆-C₁₈ aryl, a 5-20 atom heteroaryl, halo, or —CN.

R²¹ and R²², each independently, are selected from H, a C₁-C₆ alkyl, a C₃-C₁₈ cycloalkyl, a C₆-C₁₈ aryl, a 5-20 atom heteroaryl, halo, —CN, —(Ar²¹)_{d}-G, or —Ar²², provided that at least one of R²¹ and R²² is —(Ar²¹)_{d}-G or Ar²²;

Ar²¹, for each occurrence independently, is phenyl optionally substituted with one to four C₁-C₆ alkyls;

Ar²², for each occurrence independently, is:

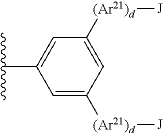

and is optionally substituted with one to three C₁-C₆ alkyls;

d, for each occurrence independently, is 0, 1, or 2;

J, for each occurrence independently, is C₆-C₁₈ aryl or 5-20 atom heteroaryl, provided that J is not unsubstituted phenyl;

G, for each occurrence independently, is C₆-C₁₈ aryl or 5-20 atom heteroaryl, provided that G is not triazinyl, pyrimidinyl, tetrazolyl, oxadiazolyl, diazanaphthyl, or pentafluorophenyl, further provided that if G is phenyl it is not unsubstituted and is not substituted with carbonyl, trifluoromethyl, triazinyl, pyrimidinyl, tetrazolyl, oxadiazolyl, diazanaphthyl, or pentafluorophenyl.

In an example embodiment of the twenty-second aspect, the present invention is a molecule represented by one of the following structural formulas:

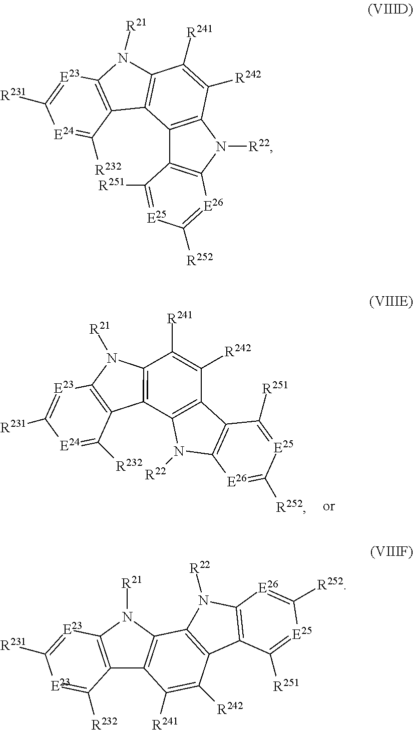

In structural formulas (VIIIA), (VIIIB), and (VIIIC) of the present invention:

R²¹ and R²², each independently, are selected from H, a C₁-C₆ alkyl, a C₃-C₁₈ cycloalkyl, a C₆-C₁₈ aryl, a 5-20 atom heteroaryl, halo, —CN, —(Ar²¹)_{d}-G, or —Ar²², provided that at least one of R²¹ and R²² is —(Ar²¹)_{d}-G or Ar²²;

Ar²¹, for each occurrence independently, is phenyl optionally substituted with one to four C₁-C₆ alkyls;

Ar²², for each occurrence independently, is:

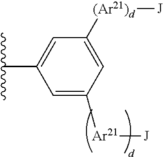

and is optionally substituted with one to three C₁-C₆ alkyls;

d, for each occurrence independently, is 0, 1, or 2;

J, for each occurrence independently, is C₆-C₁₈ aryl or 5-20 atom heteroaryl, provided that J is not unsubstituted phenyl;

G, for each occurrence independently, is C₆-C₁₈ aryl or 5-20 atom heteroaryl, provided that G is not triazinyl, pyrimidinyl, tetrazolyl, oxadiazolyl, diazanaphthyl, or pentafluorophenyl, further provided that if G is phenyl it is not unsubstituted and is not substituted with carbonyl, trifluoromethyl, triazinyl, pyrimidinyl, tetrazolyl, oxadiazolyl, diazanaphthyl, or pentafluorophenyl;

E²³, E²⁴, E²⁵, and E²⁶ are, each independently, CR^{Y} or N, wherein R^{Y}, for each occurrence independently, is H, a C₁-C₆ alkyl, a C₃-C₁₈ cycloalkyl, a C₆-C₁₈ aryl, a 5-20 atom heteroaryl, halo, or —CN; and

R²³¹, R²³², R²⁴¹, R²⁴², R²⁵¹, and R²⁵², each independently, are H, a C₁-C₆ alkyl, a C₃-C₁₈ cycloalkyl, a C₆-C₁₈ aryl, a 5-20 atom heteroaryl, halo, or —CN.

In a twenty-second aspect, the present invention is an organic light-emitting device comprising a first electrode, a second electrode, and an organic layer disposed between the first electrode and the second electrode. In an example embodiment, the organic layer comprises a molecule from any one of the one through eighteen aspects of the present invention described above. In another example embodiment, the organic layer comprises at least one light-emitting molecule represented by a structural formula selected from Table M, N, N′, N″, N′″, O, Q, Q′, B, R, or R′. In yet another example embodiment, the organic layer comprises at least one light-emitting molecule represented by any one of the structural formulas in Table M, N, N′, N″, N′″, O, Q, Q′, B, R, or R′.

In an example embodiment of any one of the one through twenty-second aspects of the present invention described above, the moiety A and the moiety D are different.

In an example embodiment of any one of the one through twenty-second aspects of the present invention described above, the moiety D has a highest occupied molecular orbital (HOMO) energy above −6.5 eV and the moiety A has a lowest unoccupied molecular orbital (LUMO) energy below −0.5 eV.

In an example embodiment of any one of the one through twenty-second aspects of the present invention described above, the molecule is group symmetric or synthetic symmetric.

### Combinatorial Assembly and Screening

Example molecules of the present invention having desirable properties, such as color of visible emission, can be constructed from the acceptor, donor, and bridge moieties described above using a combinatorial process described below. While only a few example compounds are illustrated below, it is understood that different combinations of different moieties can be used to create a combinatorial library of compounds. The example moieties below are intended only to illustrate the concepts herein, and are not intended to be limiting.

In the first step, a library of chemical moieties are screened for their abilities to function as acceptor or donor moieties. Example properties examined include desirable quantum mechanical computations such as the ionization potential of the highest occupied molecular orbital (i.e., a “donor” moiety) and the electron affinity of the lowest unoccupied molecular orbital (i.e., an “acceptor” moiety). In an example embodiment, a donor moiety can be selected if it is calculated that it has an ionization potential of greater than or equal to −6.5 eV. In another example embodiment, an acceptor moiety can be selected if it is calculated that it has an electron affinity of less than or equal to −0.5 eV. An example donor moiety selected after screening could be:

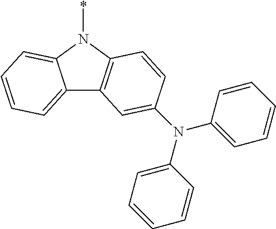 [SMILES: *n1c2ccccc2c2cc(N(c3ccccc3)c3ccccc3)ccc21]

and an example acceptor moiety selected after screening could be:

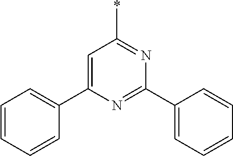 [SMILES: *c1cc(-c2ccccc2)nc(-c2ccccc2)n1]

wherein (*) represents a point of attachment for the donor and acceptor moieties either to each other or to a bridge moiety.

In a second, optional, step, if the selected donor and/or acceptor is “multi-site,” the multi-site donor moiety is combined with a single-site bridge moiety, and/or the multi-site acceptor moiety is combined with a single-site bridge moiety. If the donor and/or acceptor moieties are “single-site” moieties, then multi-site bridge moieties can be combined with the selected moieties. For the purposes of the combinatorial assembly, the number of “sites” refers to how many potentially different moieties can be attached. For example, the moiety below has one “site”:

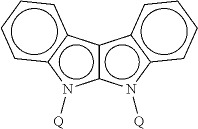

because all moieties attached at the position labeled Q must be the same. Similarly, the moiety below has two “sites” because Q and M can be the same or different:

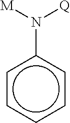

Thus, the nitrogen atom in the molecule is “multi-site.”

In the example moieties from the first step, both moieties are single-site. An example “multi-site” bridge could be:

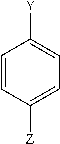

wherein the moieties attached at Y and Z are different. If the donor moiety combines with a bridge, and the acceptor combines with a bridge, the following moieties are created:

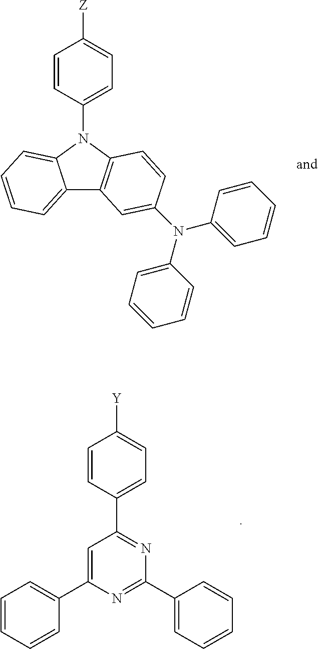

In a third step, the second step can be repeated to continuously add bridge moieties to the molecule. The only limitation is the size of final molecules that are going to be generated. The bridge molecules can be added at position Y or Z, indicated above, and can be the same bridge moiety, or a different bridge moiety. In one example embodiment, the number of bridge moieties can be limited to a number between 0 and 3. In another example, the number of donor moieties and acceptor moieties, or the total molecular weight of the molecule can be limited. In an example embodiment, the molecules are symmetrical. The symmetry can be used to limit the molecules in the combinatorial process to those that are stable. Therefore, for example, an additional bridge moiety added to the moieties from step two could be:

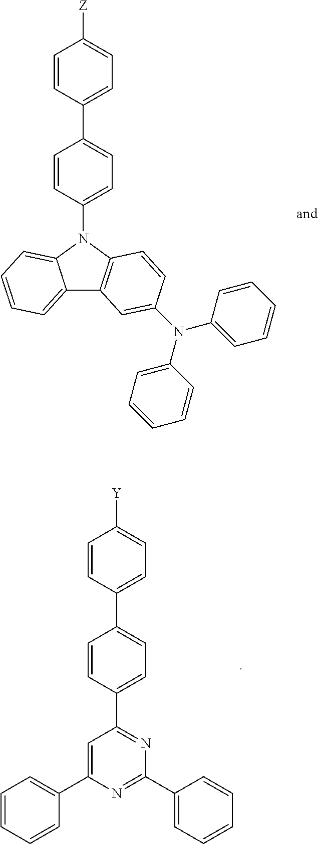

In a fourth step, the unattached point on the bridge moieties only combine with either (1) a donor moiety or an acceptor moiety that does not have a bridge moiety attached; or (2) other bridge moieties that is attached to either an acceptor moiety or a donor moiety such that the size limitation in step three is not violated, and that each molecule comprises at least one donor moiety and one acceptor moiety.

Using the example moieties and the rules described above, the following example molecules can be created:

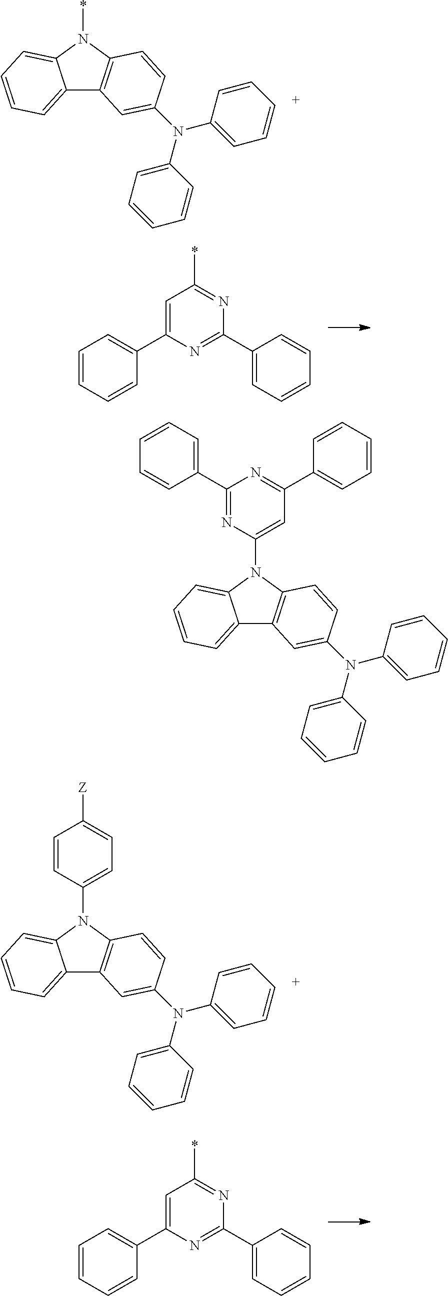

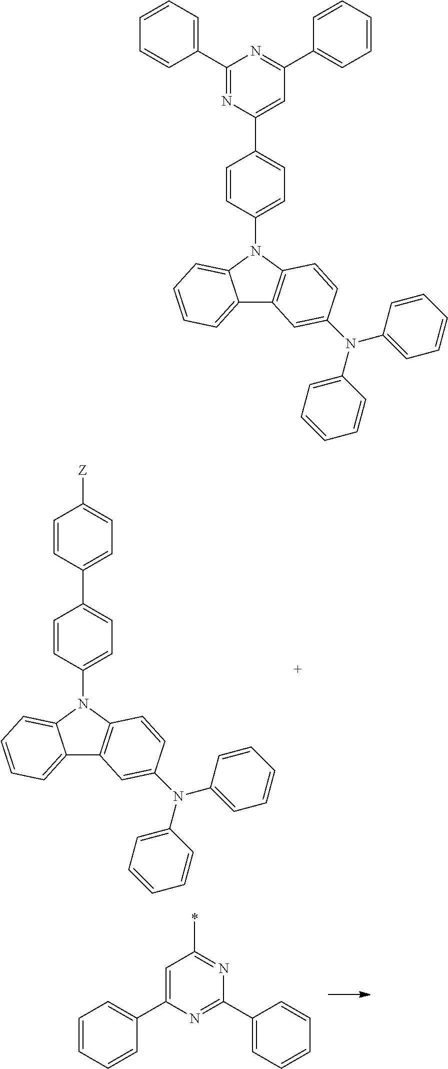

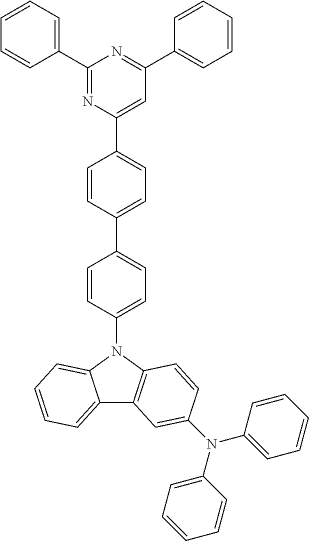 [SMILES: c1ccc(-c2cc(-c3ccc(-c4ccc(-n5c6ccccc6c6cc(N(c7ccccc7)c7ccccc7)ccc65)cc4)cc3)nc(-c3ccccc3)n2)cc1]

In the fifth step, the combined potential donors, acceptors, and bridges can be screened based on quantum mechanical computations such as desired HOMO and LUMO values, as well as vertical absorption (the energy required to excite the molecule from the ground state to the excited state), rate of decay (S1 to S0 oscillator strength, e.g., how fast and/or how bright the molecule's emission after excitation), estimated color of visible light emission in nanometers, and the singlet-triplet gap (the energy difference between the lowest singlet excited state, S1, the lowest triplet excited state, T1). Examples of these calculations for molecules embodied in the present invention are provided in Tables 1-10 and 12.

### Exemplification

The compounds described herein may be prepared by synthetic methods known to those of skill in the art. Provided below are exemplary reaction schemes for example embodiments of the present invention. Reactants and conditions suitable for carrying out the reactions described below can be found, for example in: PCT Publication WO2005/070916, Mansanet Ana Maria Castano, et al.; PCT Publication WO2010/050778, Sung Jin Eum et al.; PCT Publication WO2014/021569, Yu-Mi Chang et al.; PCT Publication WO2015/175678; PCT Publication WO2012/080062; U.S. Pat. No. 9,240,559, Oh et al.; U.S. Pat. No. 8,865,322; U.S. Patent Publication 2012/273766; U.S. Patent Publication 2016/006925; European Patent Publication EP2910555; Korean Patent KR101297162; J. Am. Chem. Soc. 1984, 106, 2569-2579; J. Am. Chem. Soc. 2016, 138, 1709-1716; J. Am. Chem. Soc. 2000, 122, 1822-1823; J. Org. Chem. 2013, 78, 2639-2648; J. Org. Chem. 2002, 67, 7185-7192; Org. Lett. 2004, 6, 985-987; Chem. Commun. 2015, 51, 12641-12644; Chem. Commun. 2012, 48, 5367-5369; Chem. Commun. 2014, 50, 13683-13686; Advanced Synthesis and Catalysis 2008, 350, 2653-2660; Org. Lett. 2004, 6, 985-987; Org. Lett. 2004, 6, 985-987; Gu Angew. Chem. Int. Ed. 2014, 53, 4850; Stille, J. K. Angew. Chem. Int. Ed. 1986, 25, 508; Miyaura, N.; Suzuki, A. Chem. Rev. 1995, 95, 2457; Synthesis 2005, 4, 547-550; J. Chem. Soc. Perk. 2 1985, 705-710; Synlett 2013, 24, 603-606; Advanced Synthesis and Catalysis 2009, 351, 931-937; Chem. A Eur. Journal 2016, 22, 6637-6642; Chem. A Eur. Journal 2006, 12, 2222-2234; Tetrahedron Letters 2014, 55, 6976-6978; and J. Organometallic Chem. 1987, 325, 13-24.

### Compound N44

Compound N44 may be prepared by a person of ordinary skill following Scheme 1. Starting materials S1-1, S1-7, S1-3, and S1-6 are commercially available, for instance from Acros.

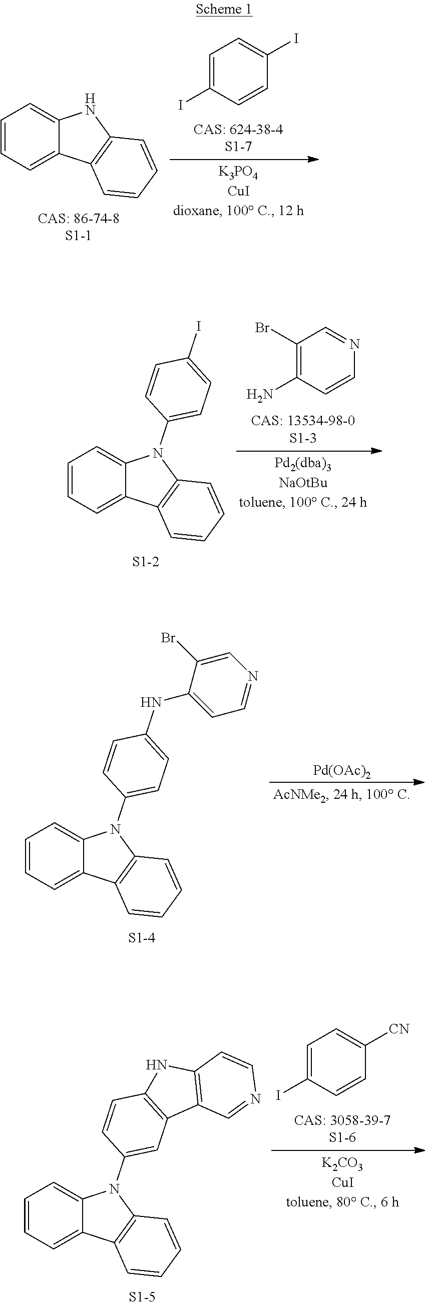

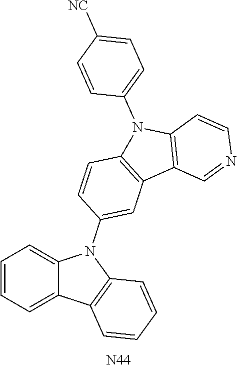 [SMILES: N#Cc1ccc(-n2c3ccncc3c3cc(-n4c5ccccc5c5ccccc54)ccc32)cc1 ; "N44"]

### Compound N34

Compound N34 may be prepared by a person of ordinary skill following Scheme 2. Starting materials S2-1, S2-2, S2-10, S2-8, and S2-9 are commercially available, for instance from Acros or Aldrich.

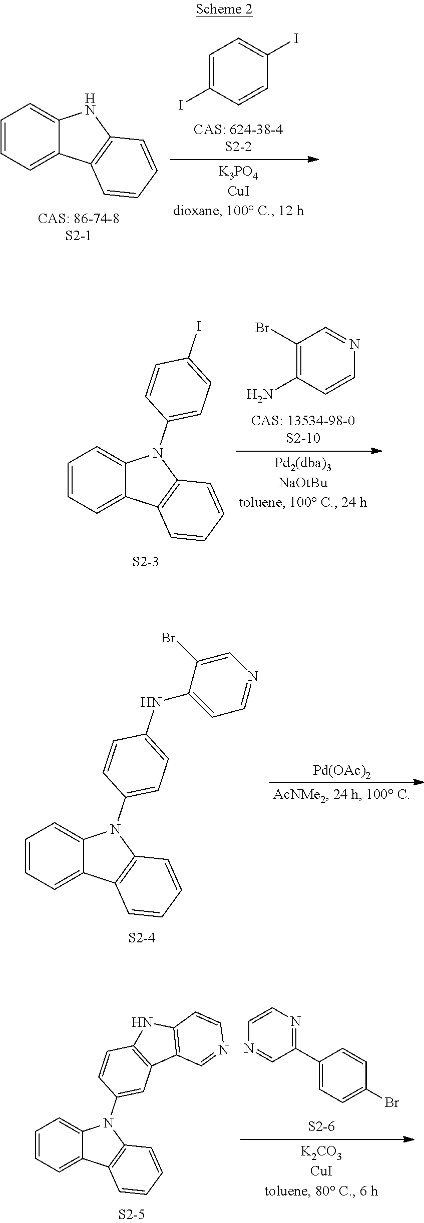

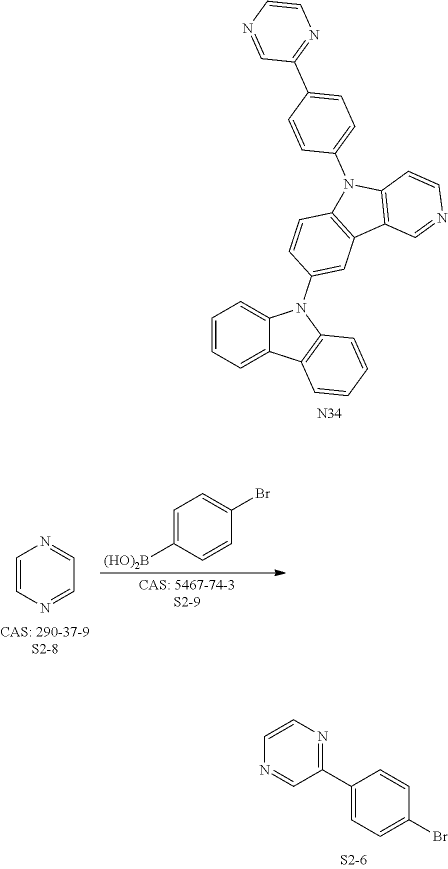

### Compound N59

Compound N59 may be prepared by a person of ordinary skill following Scheme 3. Starting materials S3-1, S3-2, S3-5, and S3-8 are commercially available, for instance from Acros or Arkpharm.

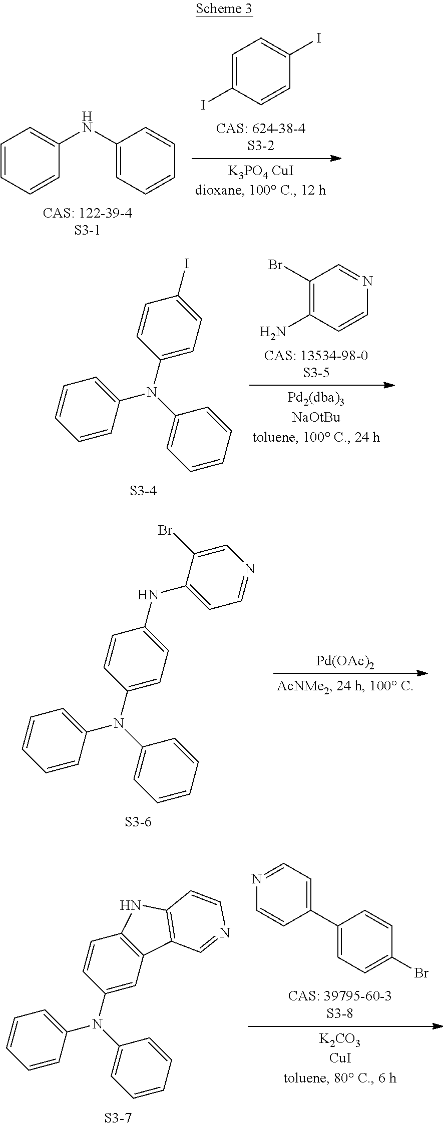

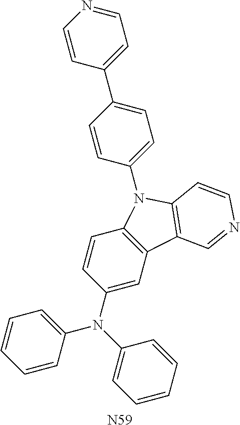 [SMILES: c1ccc(N(c2ccccc2)c2ccc3c(c2)c2cnccc2n3-c2ccc(-c3ccncc3)cc2)cc1 ; "N59"]

### Compound N17

Compound N17 may be prepared by a person of ordinary skill following Scheme 4. Starting materials S4-1, S4-4, and S4-5 are commercially available, for instance from Acros or Aldrich.

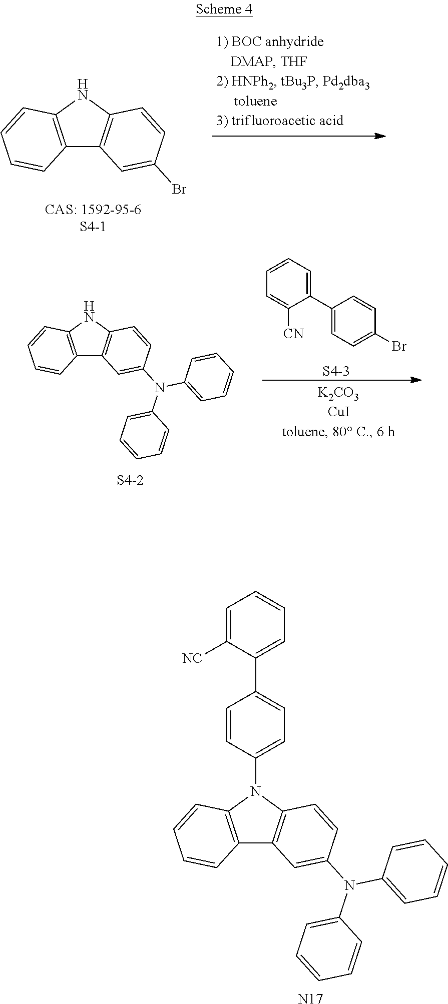

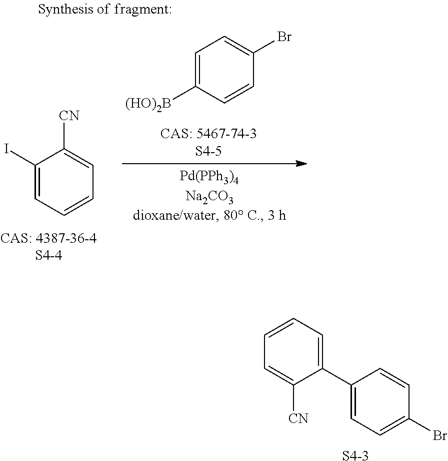

### Compound N55

Compound N55 may be prepared by a person of ordinary skill following Scheme 5. Starting materials S5-1, S5-4, and S5-5 are commercially available, for example from Acros or Aldrich.

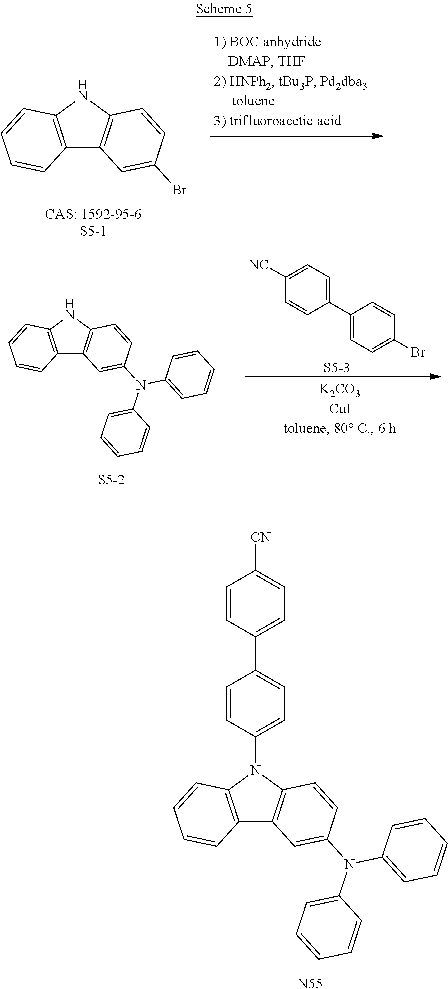

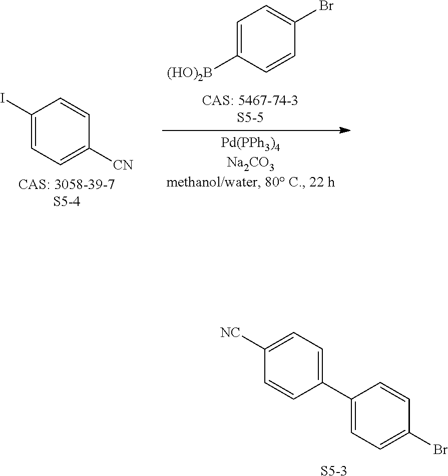

### Compound N14

Compound N14 may be prepared by a person of ordinary skill following Scheme 6. Starting materials S6-1, S6-2, S6-8, S6-9, and S6-10 are commercially available, for instance, from Acros, Aldrich, or Belpharm.

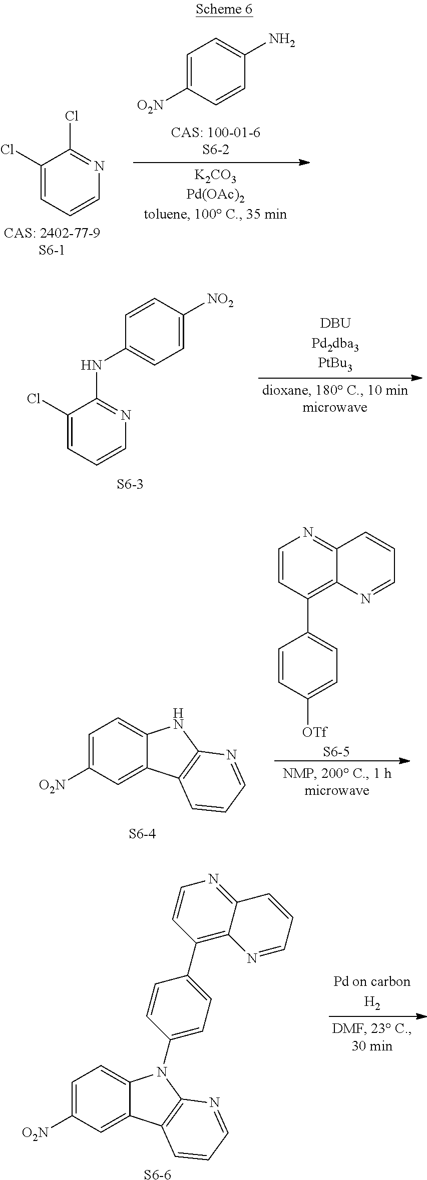

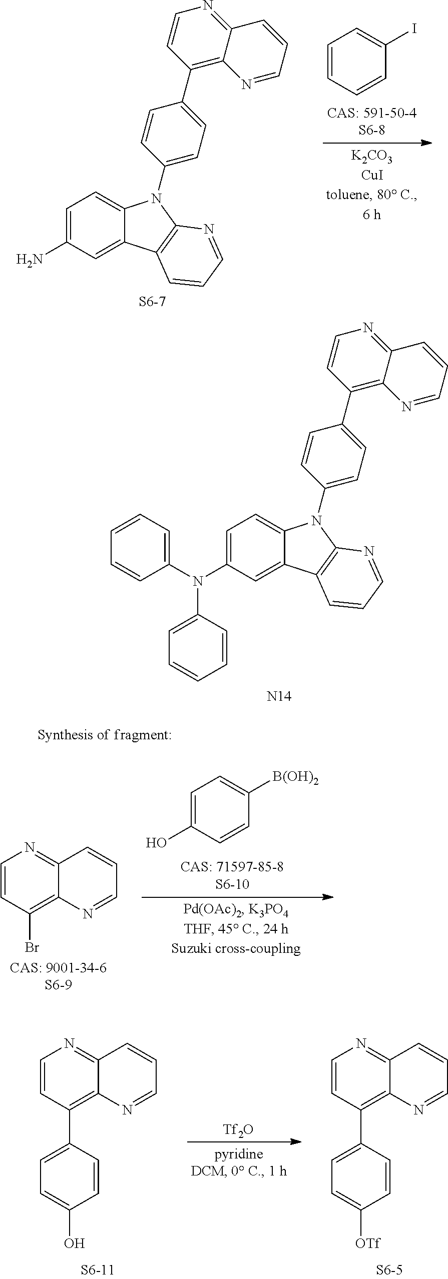

### Compound N68

Compound N14 may be prepared by a person of ordinary skill following Scheme 7. Starting materials S7-1, S7-4, and S7-5 are commercially available, for instance, from Acros or Aldrich.

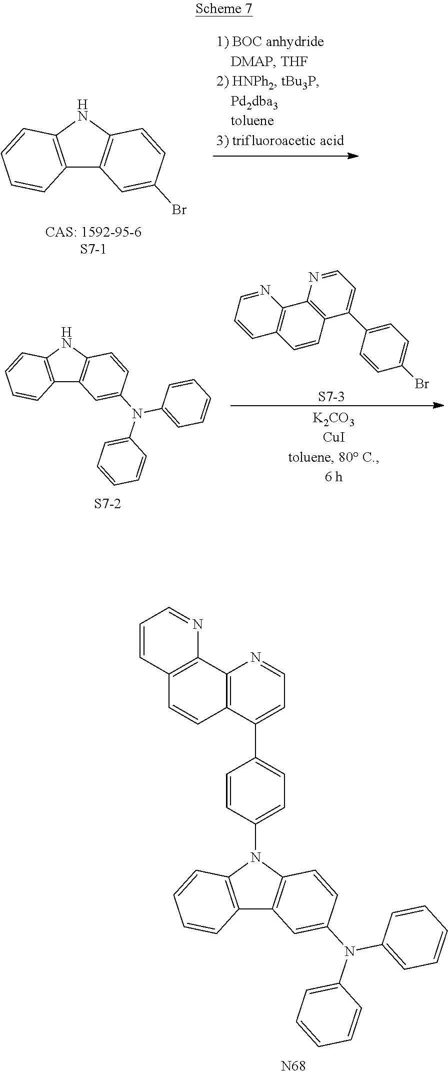

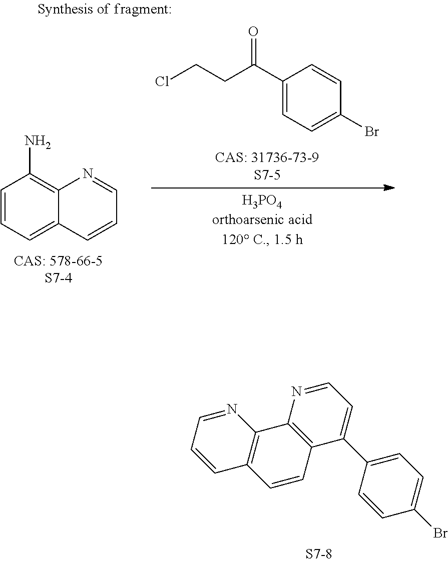 [SMILES: O=C(CCCl)c1ccc(Br)cc1 ; Nc1cccc2cccnc12 ; Brc1ccc(-c2ccnc3c2ccc2cccnc23)cc1]

### Compound N144

Compound N144 may be prepared by a person of ordinary skill following Scheme 8. Starting materials S8-1, S8-3, S8-6, and S8-7 are commercially available, for instance from Aldrich or Arkpharm.

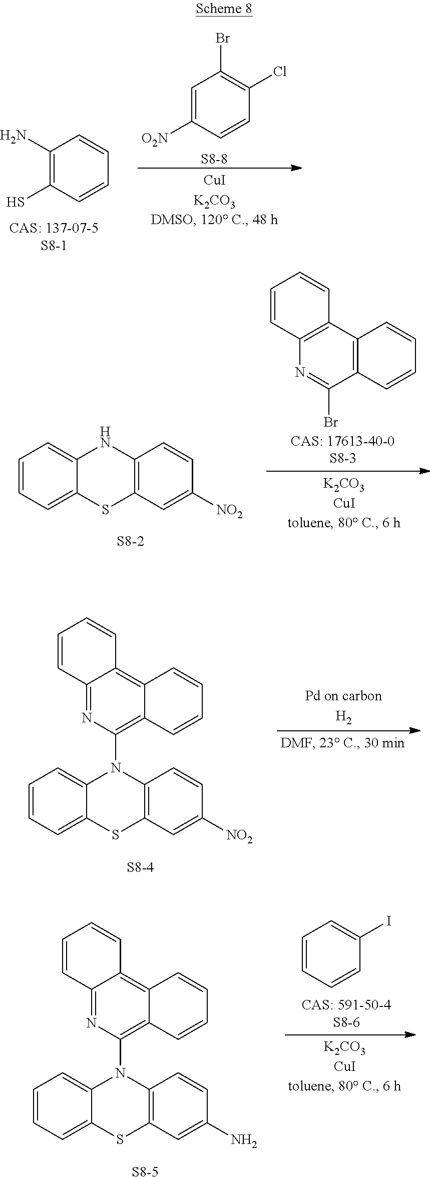

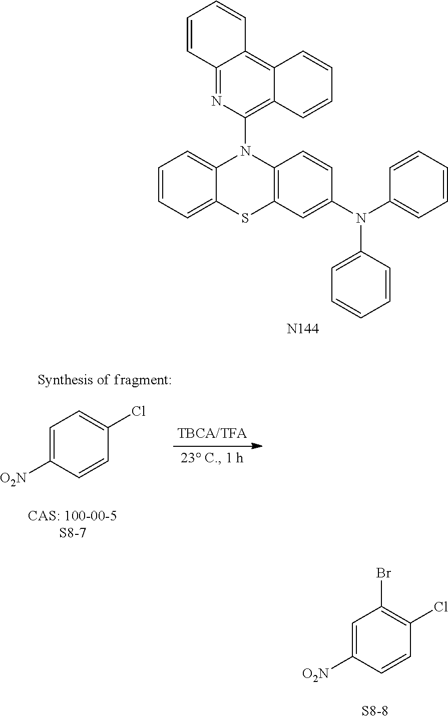

### Compound N128

Compound N128 may be prepared by a person of ordinary skill following Scheme 9. Starting materials S9-1, S9-2, S9-5, S9-8, S9-9 are commercially available, for example, from ArkPharm, Aldrich, or Acros.

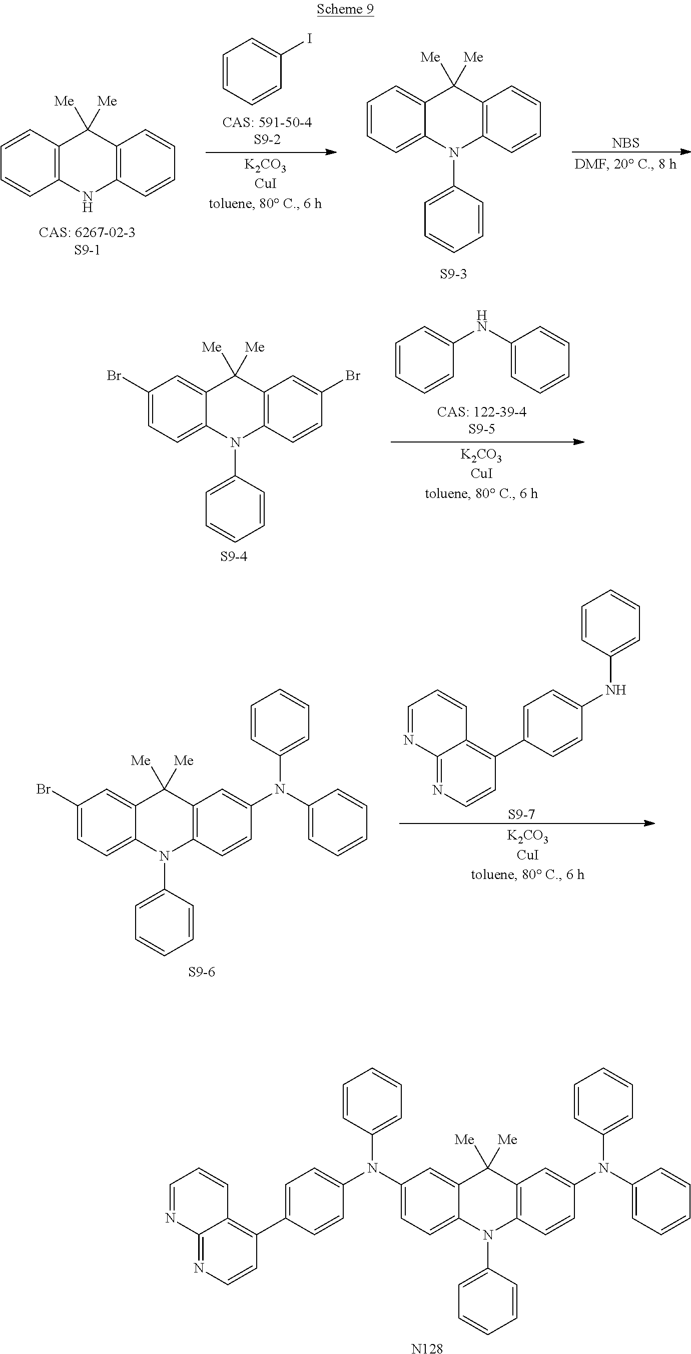

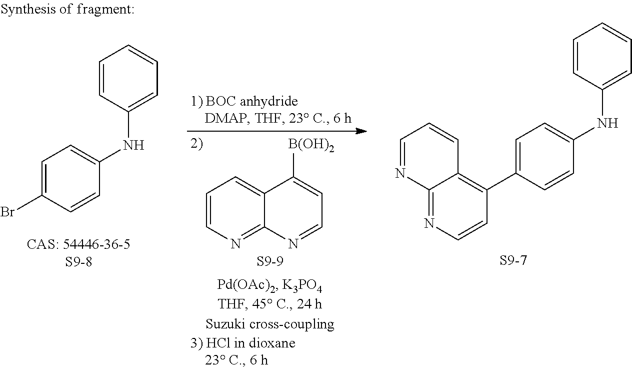

### Compound N53

Compound N53 may be prepared by a person of ordinary skill following Scheme 10. Starting materials S10-1, S10-3, and S10-7 are commercially available, for instance from Aldrich or Arkpharm.

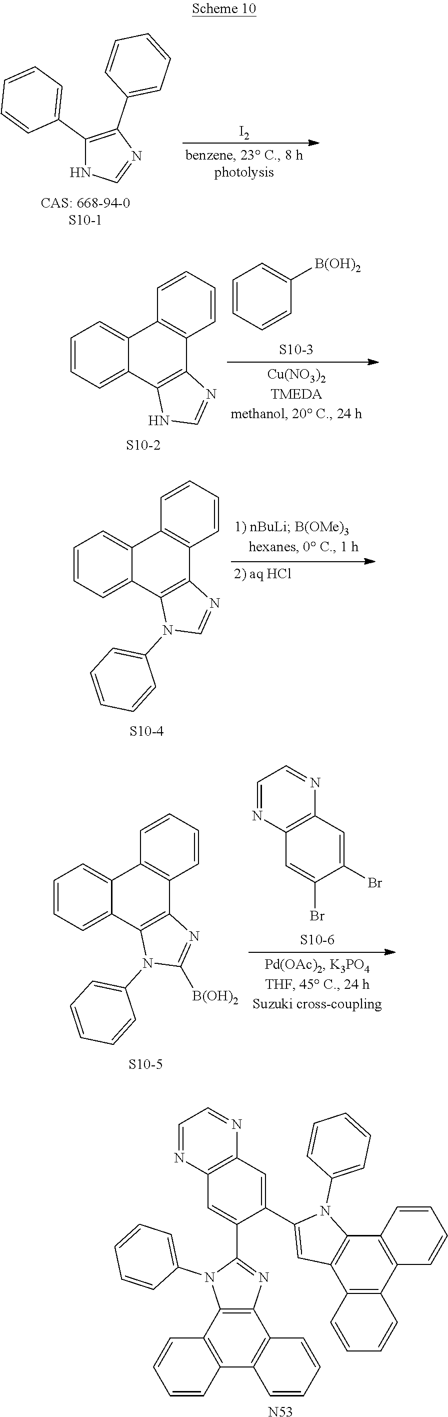

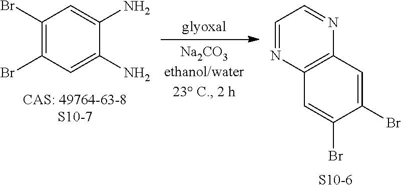 [SMILES: Nc1cc(Br)c(Br)cc1N ; Brc1cc2nccnc2cc1Br]

### Compound N92

Compound N92 may be prepared by a person of ordinary skill following Scheme 11. Starting materials S11-1, S11-4, and S11-5 are commercially available, for instance from Aldrich.

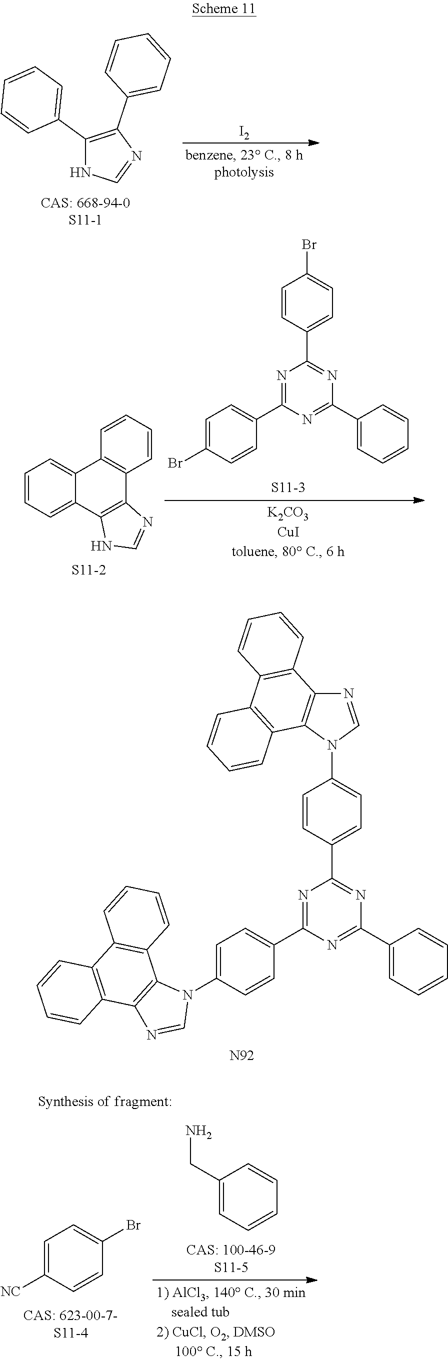

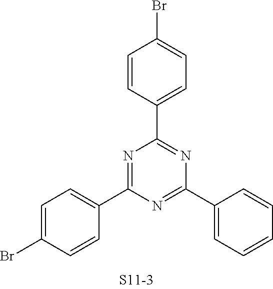 [SMILES: Brc1ccc(-c2nc(-c3ccccc3)nc(-c3ccc(Br)cc3)n2)cc1]

### Compound Q11

Compound Q11 may be prepared by a person of ordinary skill following Scheme 12. Starting materials S12-1 and S12-4 are commercially available and may be purchased, for example, from Acros or Aldrich.

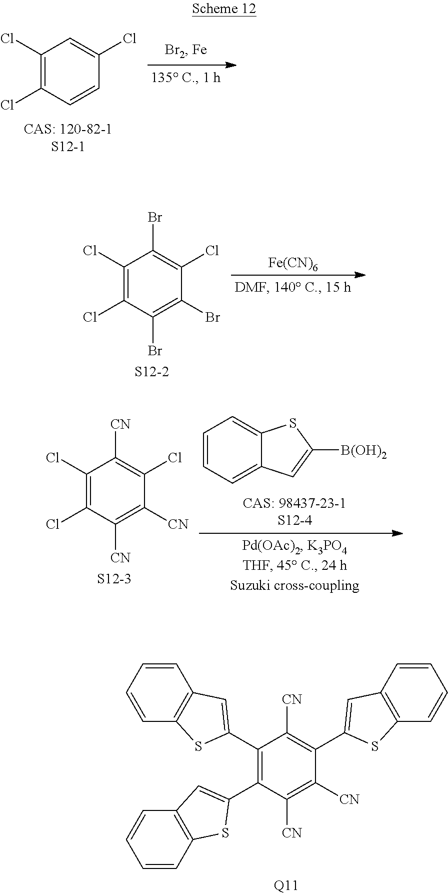

### Compound R19

Compound R19 may be prepared by a person of ordinary skill following Scheme 13. Starting materials S13-1, S13-2, and S13-5 are commercially available, for instance, from Acros or Aldrich.

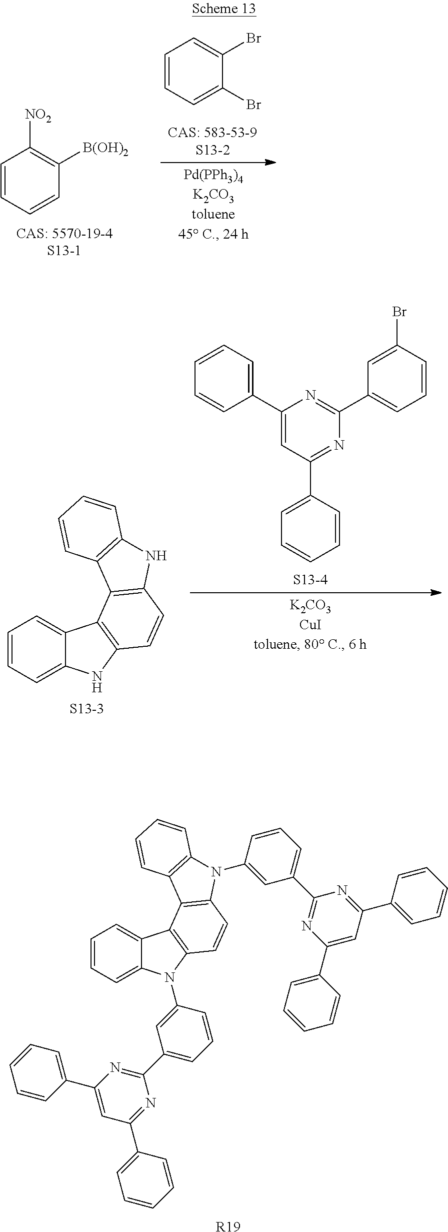

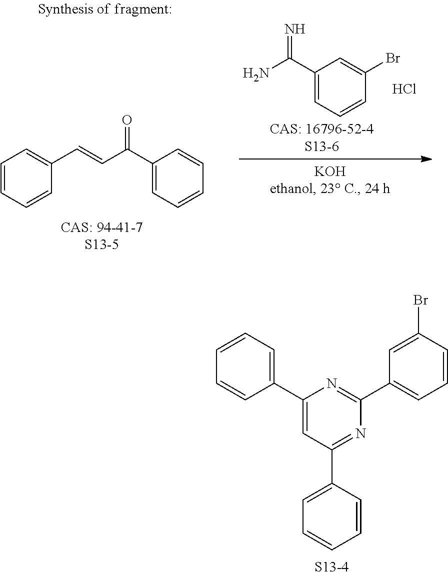 [SMILES: N=C(N)c1cccc(Br)c1 ; Cl ; O=C(/C=C/c1ccccc1)c1ccccc1 ; Brc1cccc(-c2nc(-c3ccccc3)cc(-c3ccccc3)n2)c1]

### Compound R51

Compound R51 may be prepared by a person of ordinary skill following Scheme 14. Starting materials S14-1, S14-2, S14-5, and S14-7 are commercially available, for example, from Acros or Aldrich.

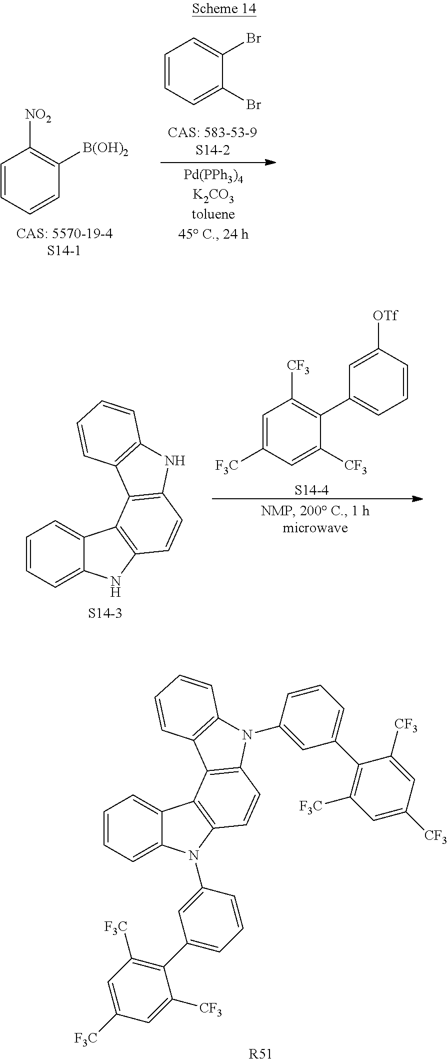

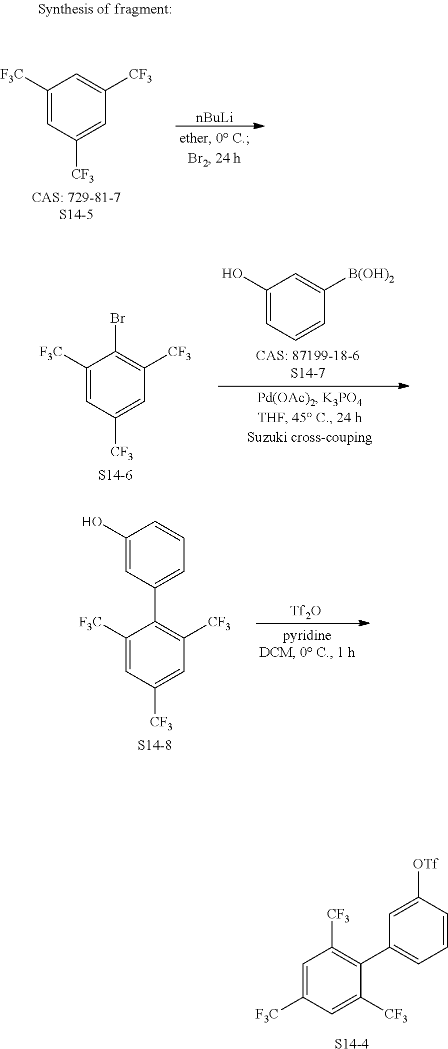

### Compound R18

Compound R18 may be prepared by a person of ordinary skill following Scheme 15. Starting materials S15-1, S15-5, S15-7, and S15-9 are commercially available and may be purchased, for example, from Acros or Aldrich.

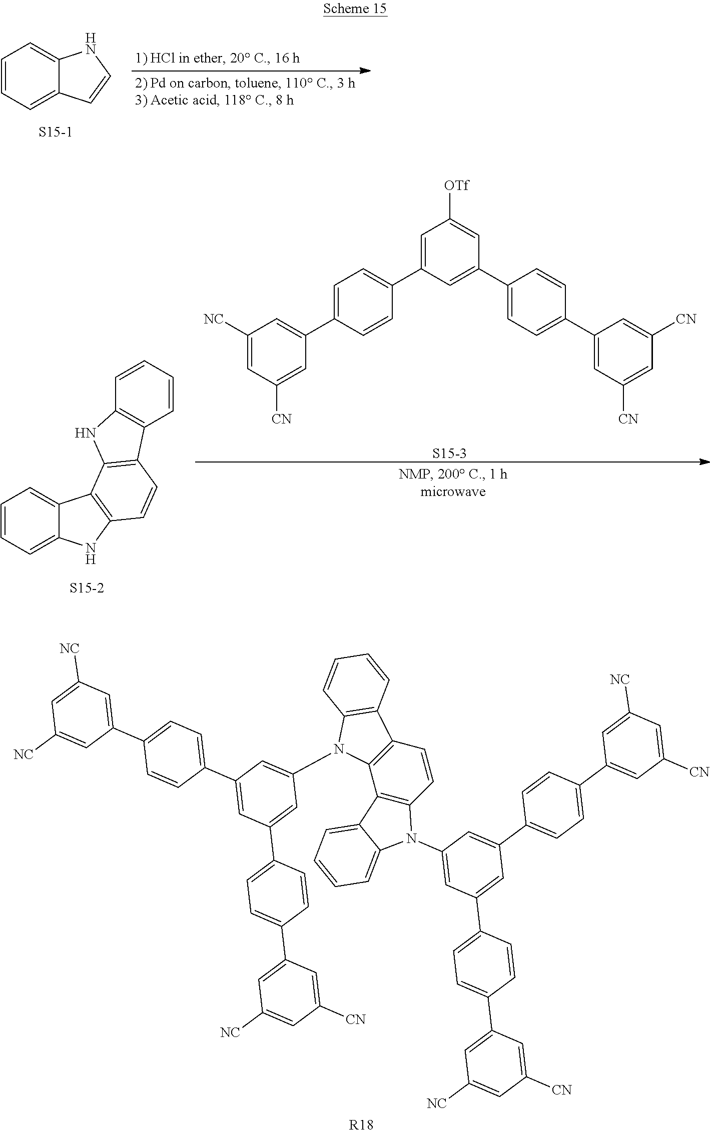

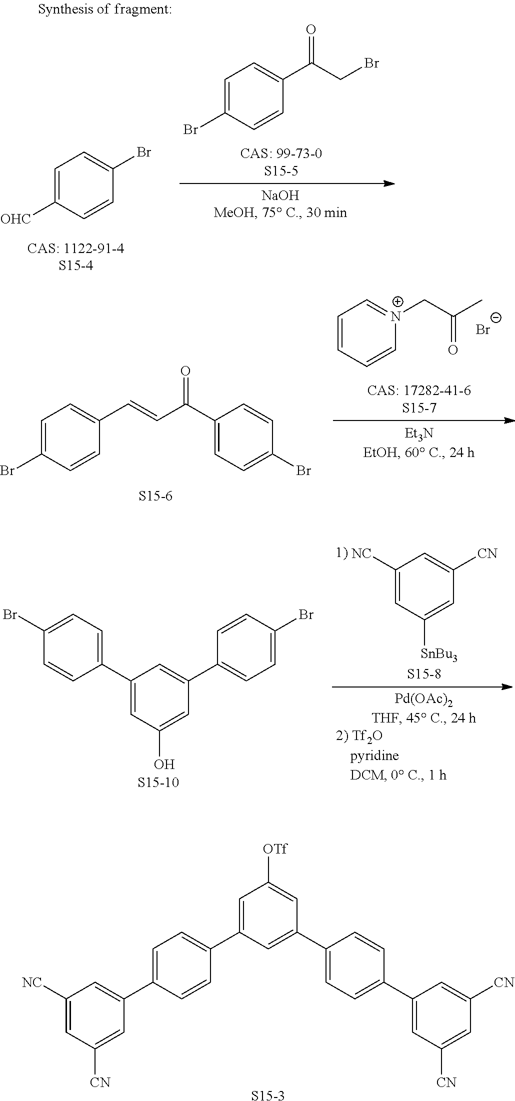

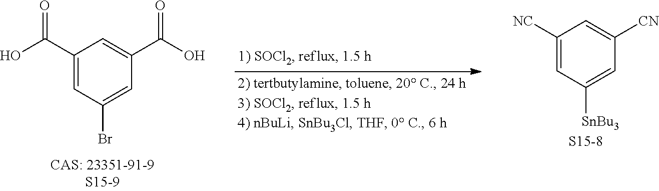

### Compound R108

Compound R108 may be prepared by a person of ordinary skill following Scheme 16. Starting materials S16-1, S16-4, S16-5, S16-8, and S16-9 are commercially available and may be purchased, for example, from Acros or Aldrich.

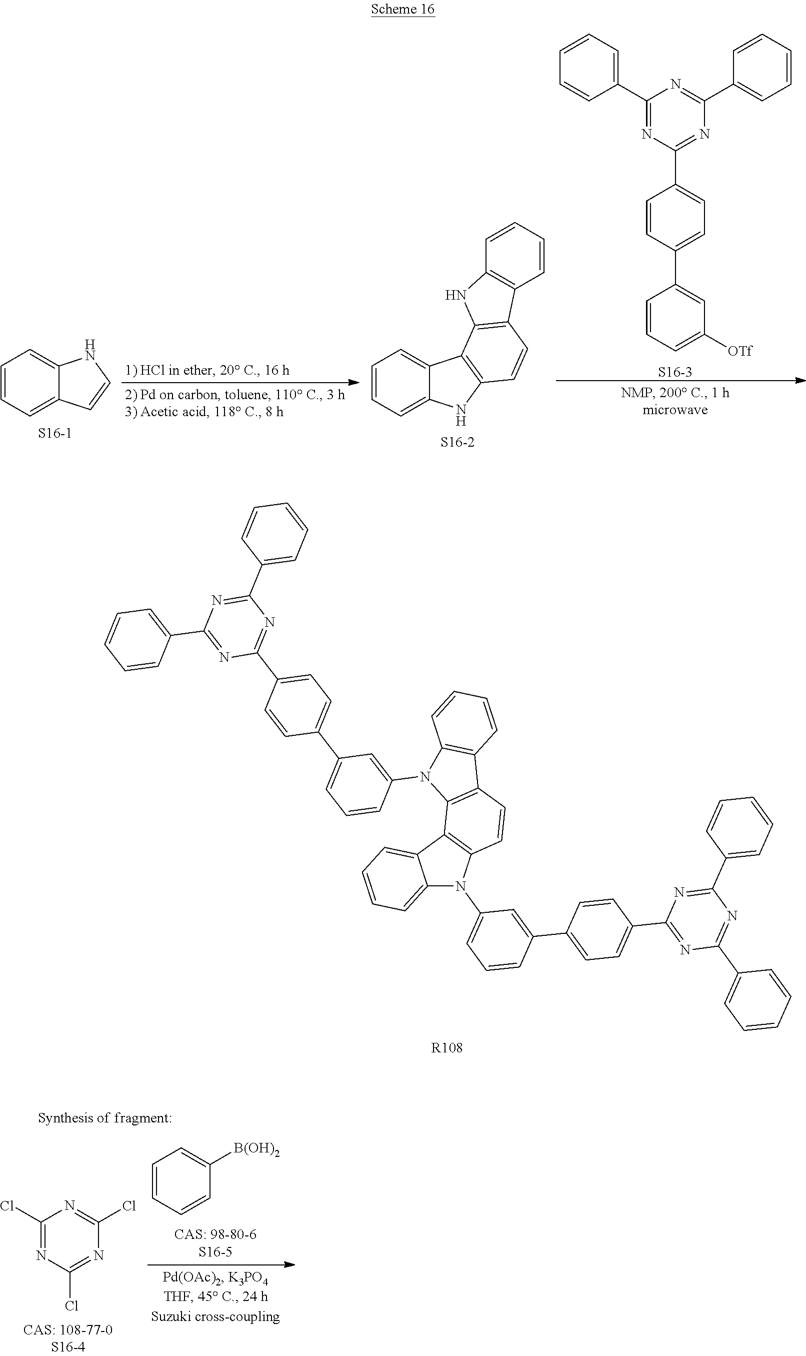

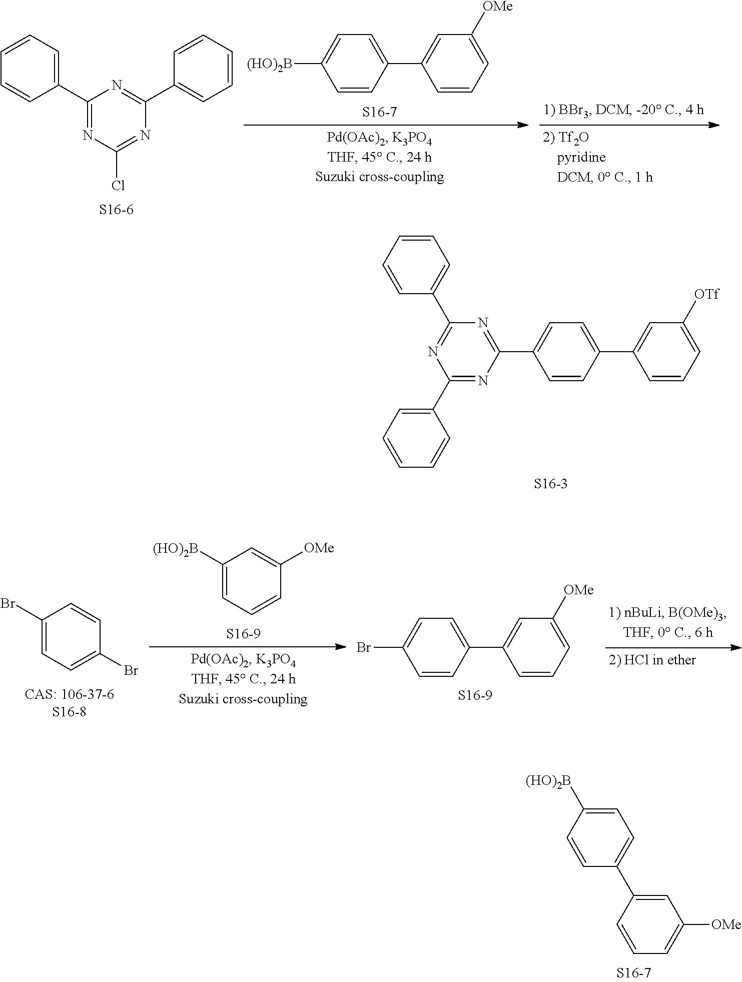

### Compound R109

Compound R109 may be prepared by a person of ordinary skill following Scheme 17. Starting materials S17-1, S17-2, S17-5, S17-6, S17-8, and S17-9 are commercially available and may be purchased, for example, from Acros or Aldrich.

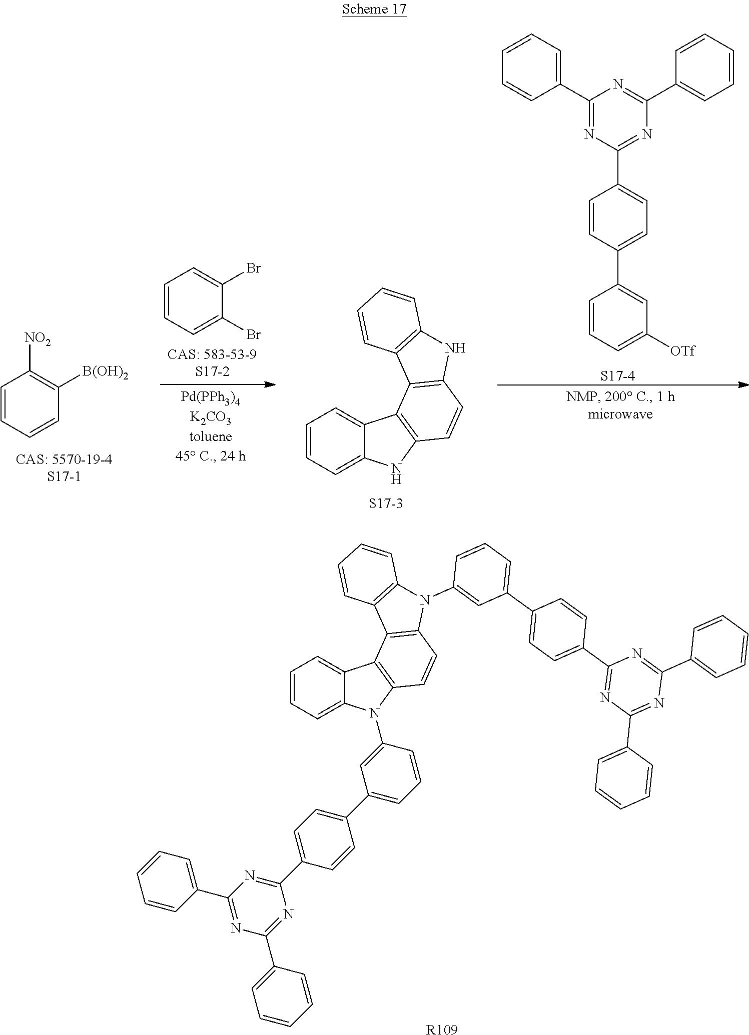

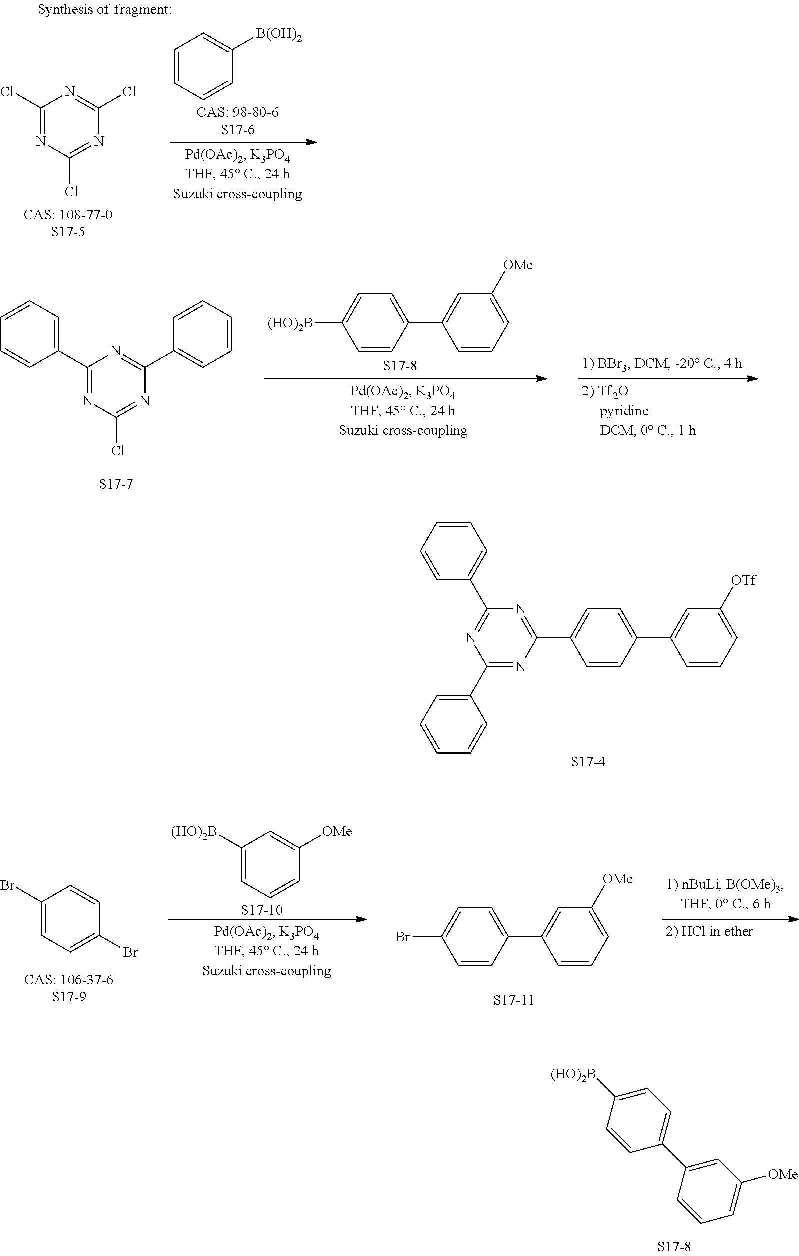

### Compound R72

Compound R72 may be prepared by a person of ordinary skill following Scheme 18. Starting materials S18-1, S18-2, S18-5, and S18-6 are commercially available and may be purchased, for example, from Acros, Aldrich, or Bepharm.

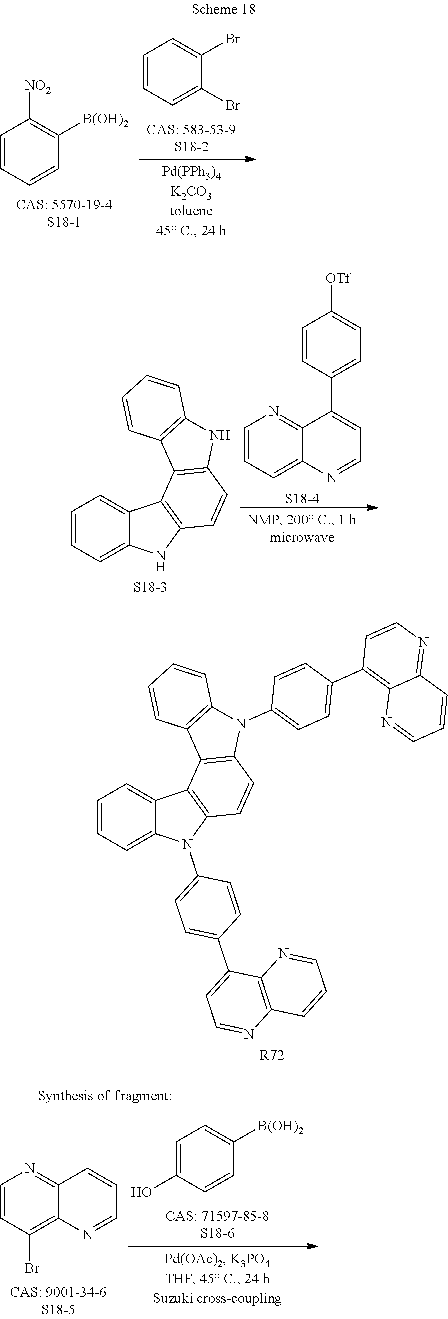

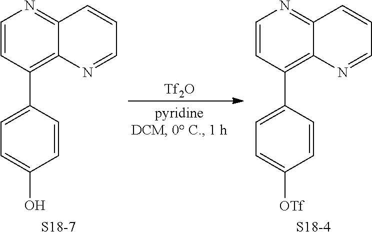

### Compound R82

Compound R82 may be prepared by a person of ordinary skill following Scheme 19. Starting materials S19-1, S19-2, S19-5, and S19-7 are commercially available, for example, from Acros or Aldrich.

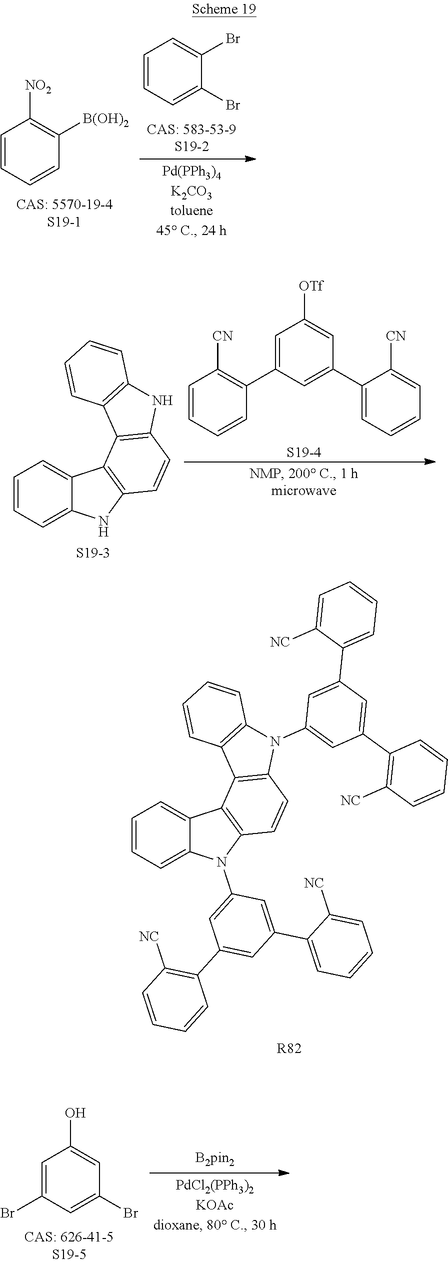

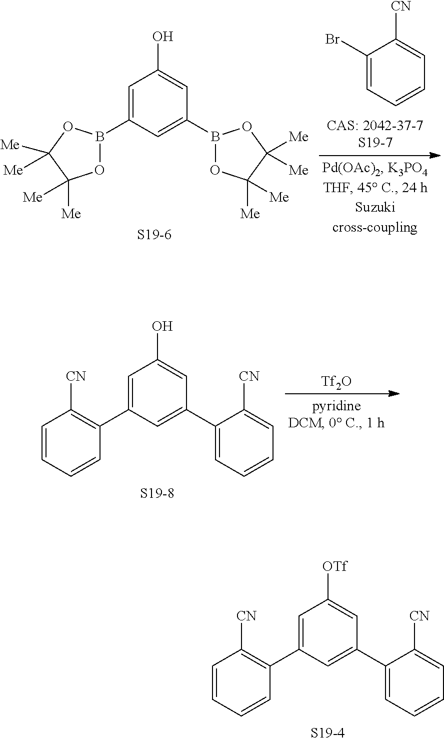

### Compound R74

Compound R74 may be prepared by a person of ordinary skill following Scheme 20. Starting materials S20-1, S20-4, and S20-6 are commercially available and may be purchased, for example, from Acros or Aldrich.

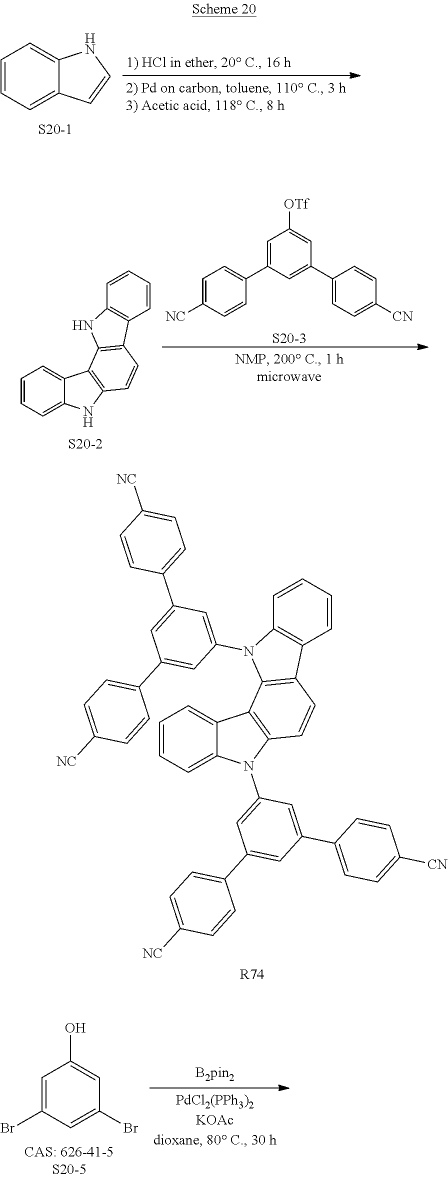

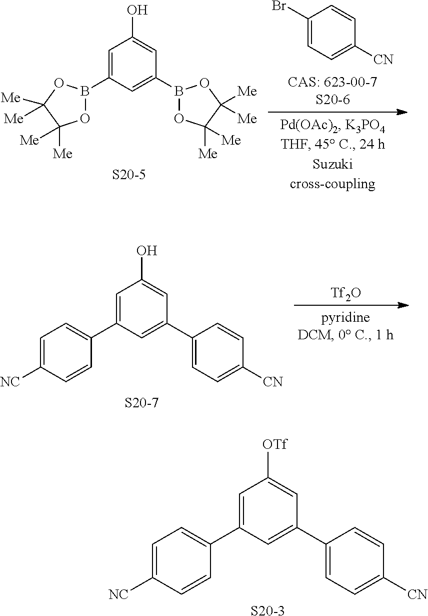

### Compound R57

Compound R57 may be prepared by a person of ordinary skill following Scheme 21. Starting materials S21-1, S21-2, S21-5, and S21-7 are commercially available, for example, from Acros, Aldrich, or Alfa-Aesar.

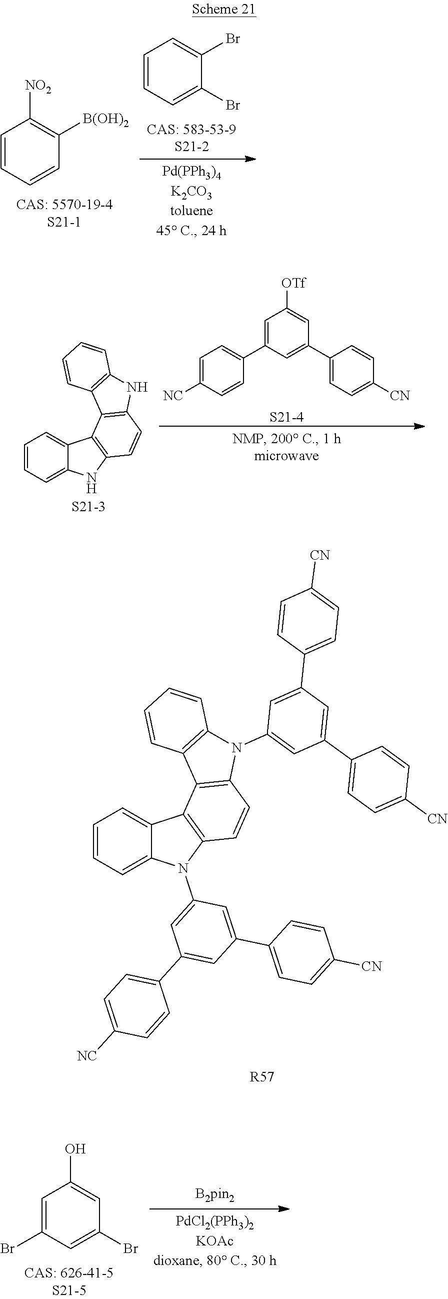

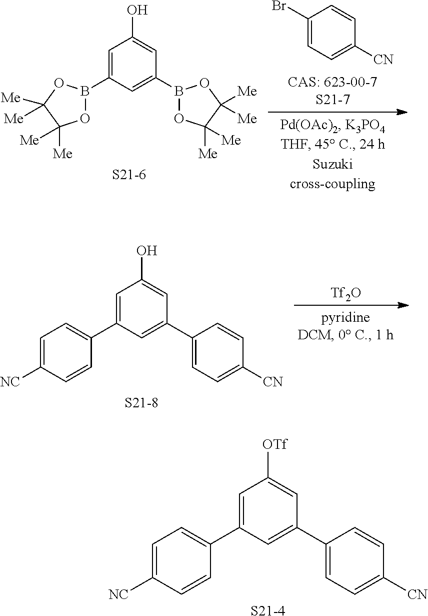

### Compound R38

Compound R38 may be prepared by a person of ordinary skill following Scheme 22. Starting materials S22-1, S22-2, S22-5, and S22-7 are commercially available, for example, from Acros, Arkpharm, or Aldrich.

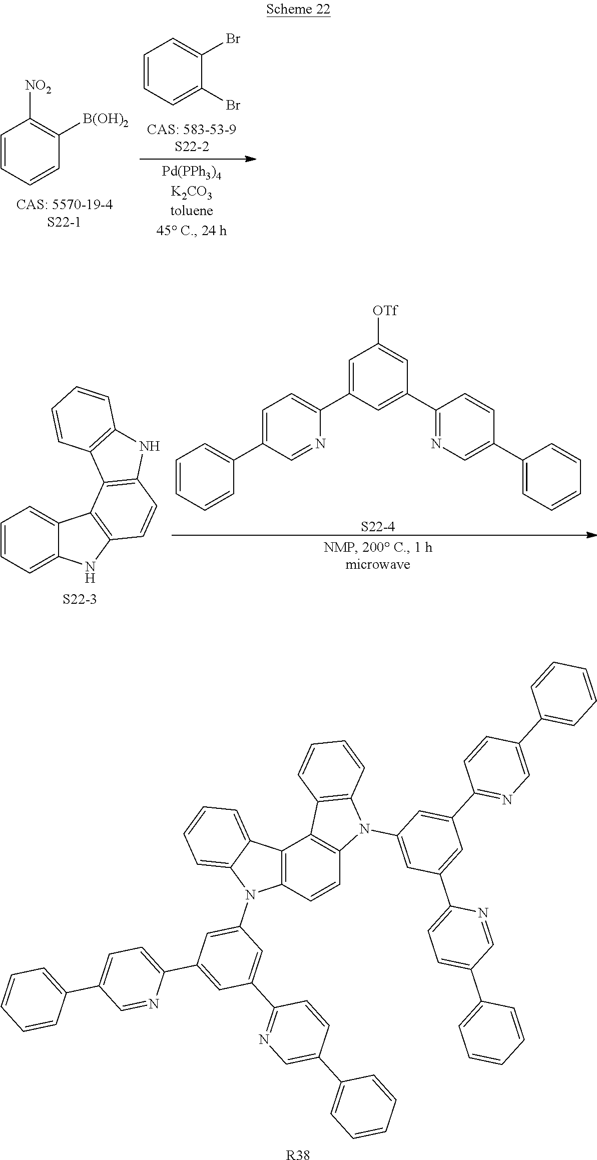

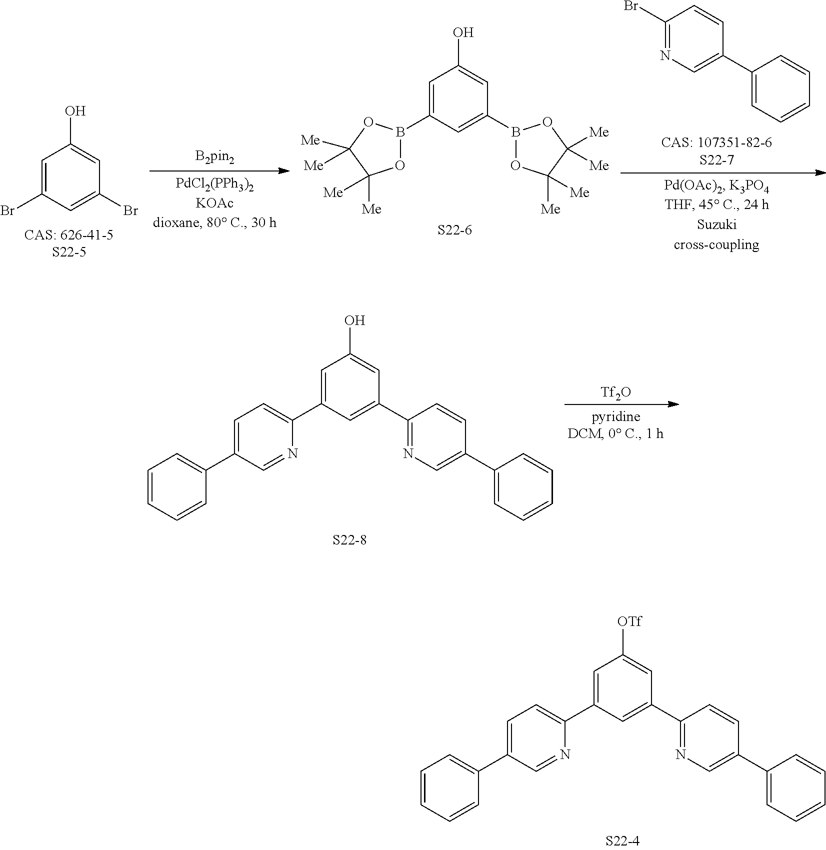

### Compound R50

Compound R50 may be prepared by a person of ordinary skill following Scheme 23. Starting materials S23-1, S23-2, S23-5, and S23-6 are commercially available, for example, from Acros, TCI America, or Aldrich.

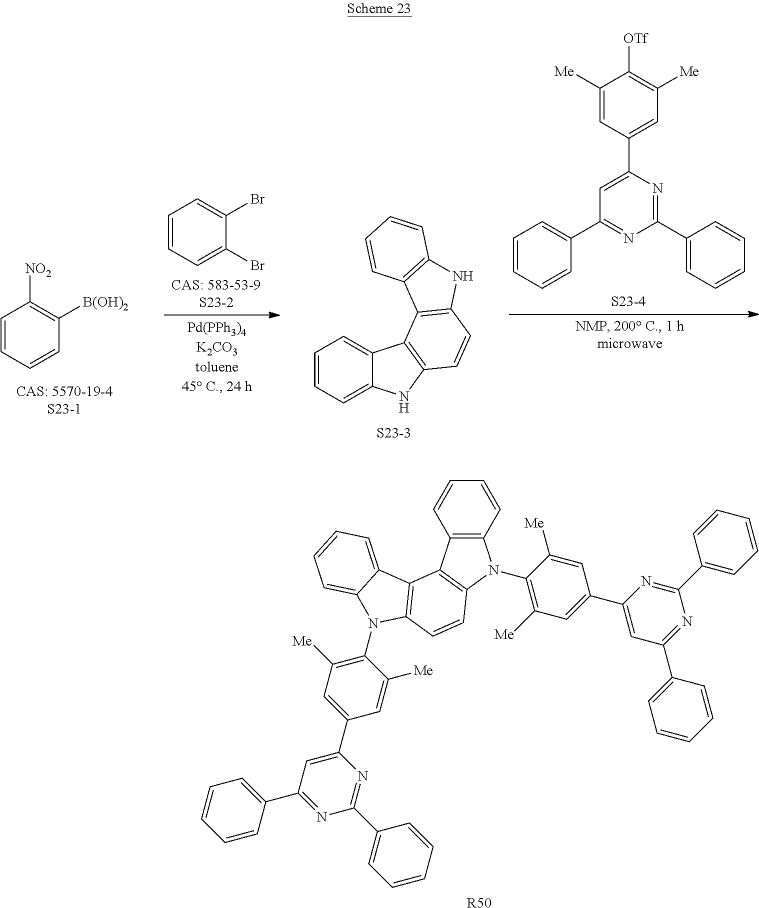

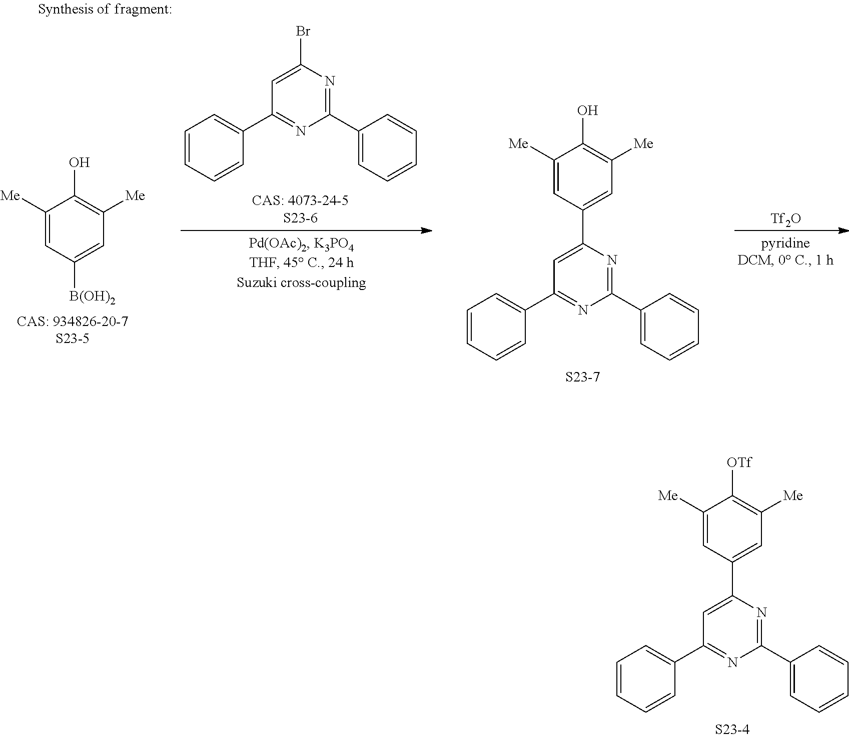 [SMILES: "Tf2O" ; Cc1cc(B(O)O)cc(C)c1O ; Brc1cc(-c2ccccc2)nc(-c2ccccc2)n1 ; Cc1cc(-c2cc(-c3ccccc3)nc(-c3ccccc3)n2)cc(C)c1O ; Cc1cc(-c2cc(-c3ccccc3)nc(-c3ccccc3)n2)cc(C)c1OS(=O)(=O)C(F)(F)F]

### Compound R110

Compound R110 may be prepared by a person of ordinary skill following Scheme 24. Starting materials S25-1, S25-2, S25-5 and S25-6 are commercially available, for example, from Acros or Aldrich.

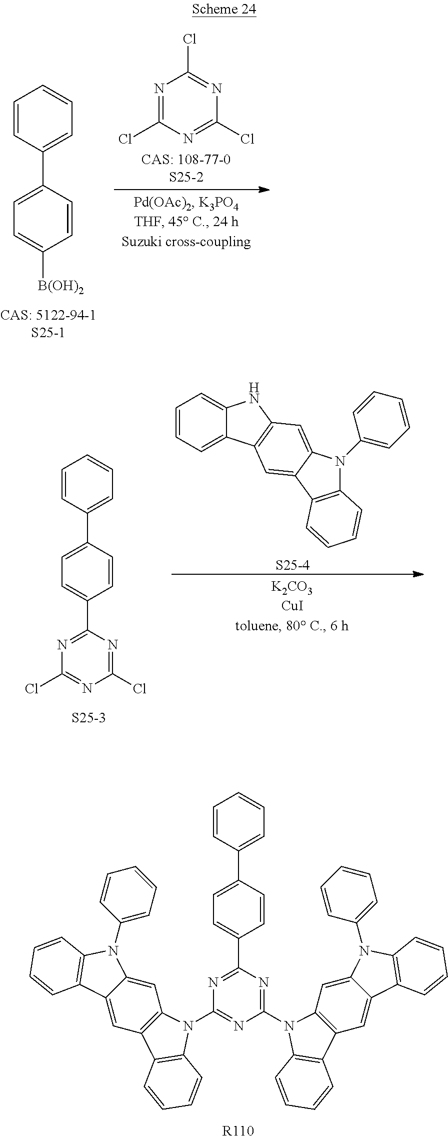

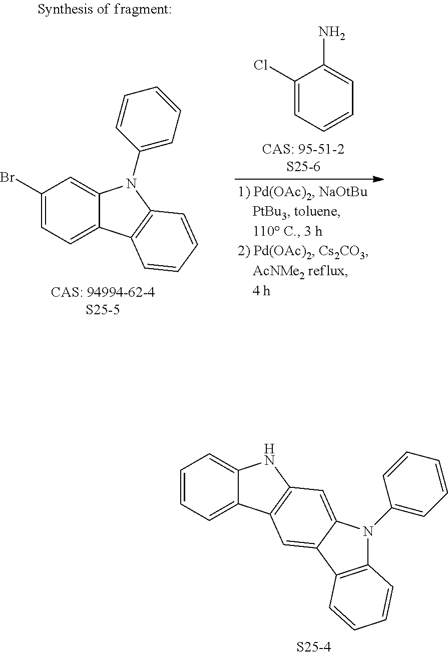 [SMILES: Brc1ccc2c3ccccc3n(-c3ccccc3)c2c1 ; Nc1ccccc1Cl ; "S25-4" ; c1ccc(-n2c3ccccc3c3cc4c(cc32)[nH]c2ccccc24)cc1]

### Compound R20

Compound R20 may be prepared by a person of ordinary skill following Scheme 25. Starting materials S27-1, S27-2, S27-5, and S27-7 are commercially available, for example, from Acros, Arkpharm, or Aldrich.

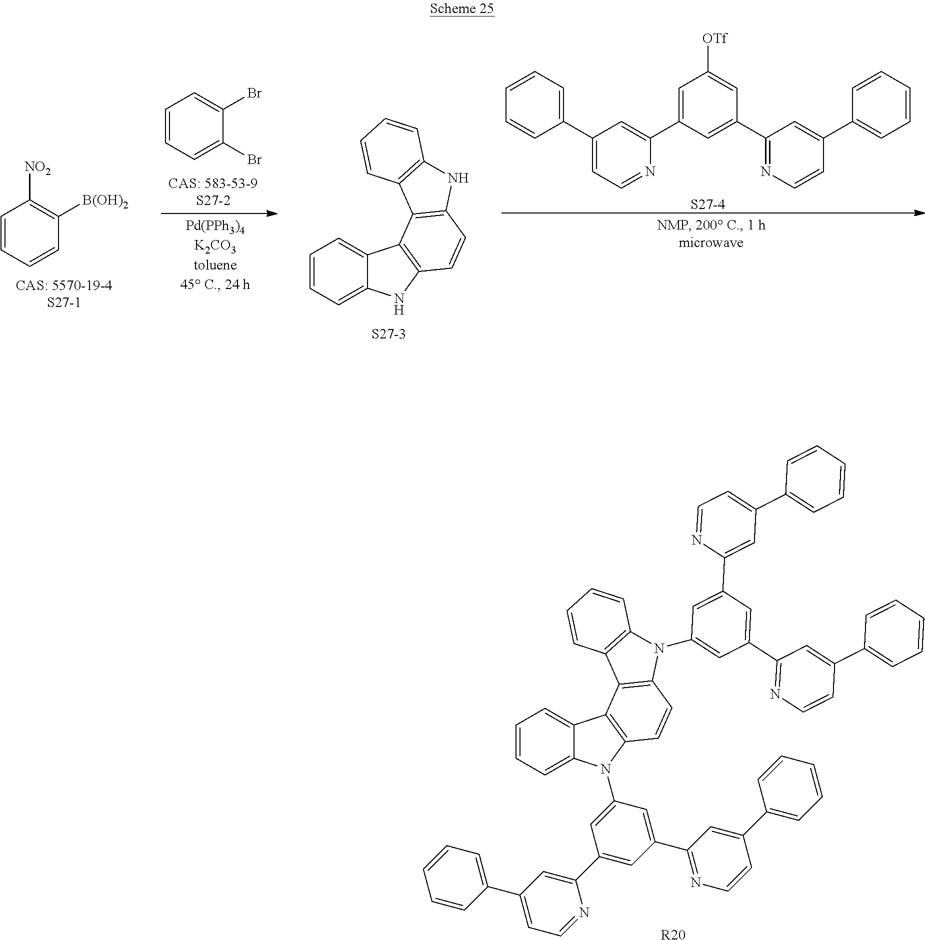

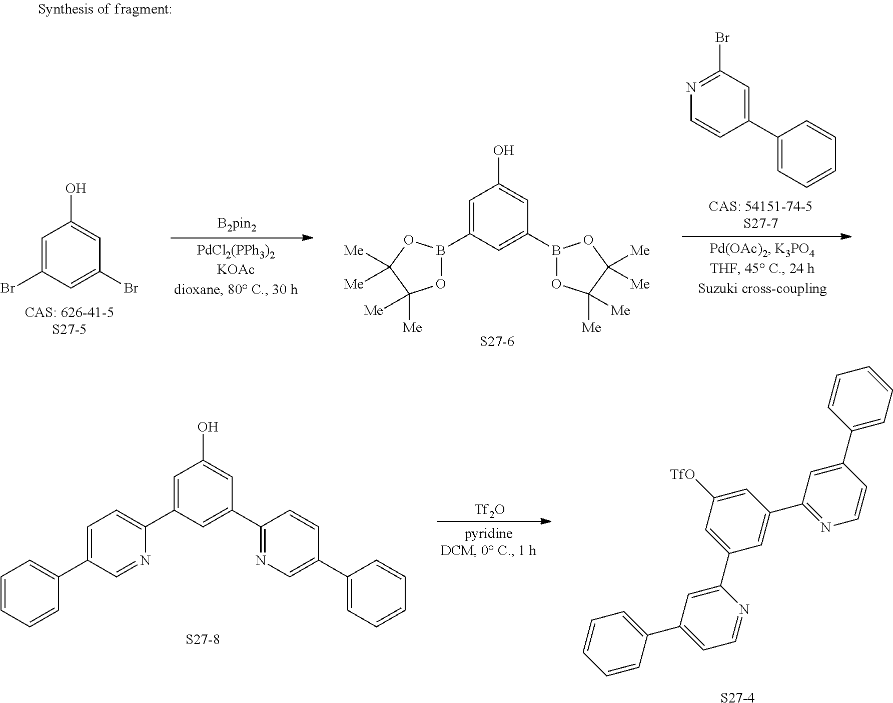

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A molecule represented by structural formula (I): 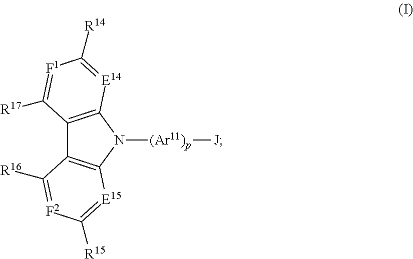
wherein:
E¹⁴, and E¹⁵ are, each independently, CR^{A} or N, wherein R^{A}, for each occurrence independently, H, a C₁-C₆ alkyl, a C₃-C₁₈ cycloalkyl, a C₆-C₁₈ aryl, a 5-20 atom heteroaryl, a halo, or —CN;
J is any moiety selected from —CN, 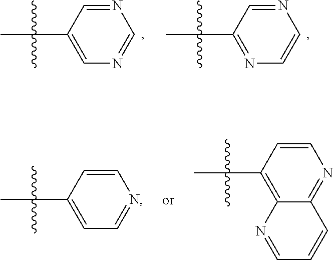 [SMILES: *c1cncnc1 ; *c1cnccn1 ; *c1ccncc1 ; "or" ; *c1ccnc2cccnc12]
and is optionally substituted with one or more R¹¹, each independently selected from C₁-C₆ alkyl, a C₃-C₁₈ cycloalkyl, a C₆-C₁₈ aryl, a 5-20 atom heteroaryl, a halo, or —CN;
R¹⁴, R¹⁵, R¹⁶, and R¹⁷ are, each independently, H, a C₁-C₆ alkyl, a C₃-C₁₈ cycloalkyl, a C₆-C₁₈ aryl, a 5-20 atom heteroaryl, a halo, or —CN;
F¹ is C—(Ar¹²)_{q}-G;
F² is CR^{B} or N, wherein R^{B} is H, a C₁-C₆ alkyl, a C₃-C₆ cycloalkyl, a C₆-C₁₈ aryl, a 5-20 atom heteroaryl, a halo, or —CN, or —(Ar¹²)_{q}-G;
Ar¹¹, for each occurrence independently, is phenyl optionally substituted with one to four C₁-C₆ alkyls;
Ar¹², for each occurrence independently, is phenyl optionally substituted with one to four C₁-C₆ alkyls;
p is 0, 1, or 2;
q is 0 or 1; and
G, for each occurrence independently, is a moiety represented by one of the following structural formulas: 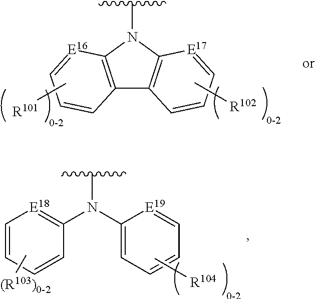
wherein:
E¹⁶, E¹⁷, E¹⁸, and E¹⁹ are, each independently, CR^{C} or N, wherein R^{C} is H, a C₁-C₃ alkyl, halo, or —CN; and
R¹⁰¹, R¹⁰², R¹⁰³, and R¹⁰⁴ are, each independently, a C₁-C₆ alkyl, a C₃-C₁₈ cycloalkyl, a C₆-C₁₈ aryl, a 5-20 atom heteroaryl, a halo, or —CN;
provided that the molecule is not represented by any structural formula in Table NN1.

2. The molecule of claim 1, wherein J is unsubstituted.

3. The molecule of claim 1, wherein J is substituted with one or more R¹¹ selected from C₁-C₆ alkyl or C₆-C₁₈ aryl.

4. The molecule of claim 3, wherein J is substituted with one or more R¹¹ selected from C₁-C₆ alkyl or phenyl.

5. The molecule of claim 1, wherein F² is CR^{B}.

6. The molecule of claim 1, wherein G, for each occurrence independently, is a moiety represented by one of the following structural formulas:

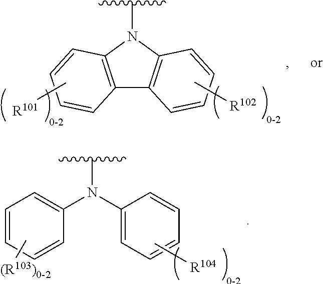

7. The molecule of claim 1 any one of the preceding claims, wherein:
R^{A}, for each occurrence independently, is H or a C₁-C₆ alkyl;
R¹⁴, R¹⁵, R¹⁶, and R¹⁷ are, each independently, H, a C₁-C₆ alkyl, or a C₃-C₆ cycloalkyl; and
F² is CR^{B}; and
R^{B} is H, a C₁-C₆ alkyl, a C₃-C₆ cycloalkyl, or —(Ar¹²)_{q}-G.

8. The molecule of claim 1, represented by the following structural formula: 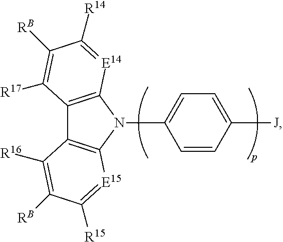
wherein:
E¹⁴, and E¹⁵, are, each independently, CR^{A} or N, wherein R^{A}, for each occurrence independently, is H or a C₁-C₆ alkyl;
p is 0 or 1;
R^{H} is a C₆-C₁₈ aryl or a 5-20 atom heteroaryl;
R¹⁴, R¹⁵, R¹⁶, and R¹⁷ are, each independently, H, a C₁-C₆ alkyl, a C₃-C₁₈ cycloalkyl, a halo, or —CN;
R^{B} is, for each occurrence independently, H, a C₁-C₆ alkyl, a C₃-C₆ cycloalkyl, or a moiety represented by one of the following structural formulas: 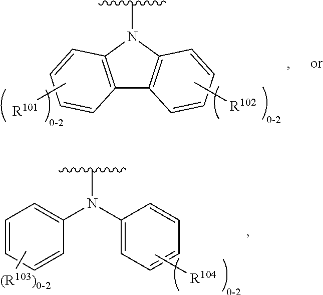
wherein:
R¹⁰¹, R¹⁰², R¹⁰³, and R¹⁰⁴ are, each independently, a C₁-C₆ alkyl or a C₃-C₆ cycloalkyl.

9. The molecule of claim 1, wherein E¹⁴ and E¹⁵ are CR^{A}.

10. The molecule of claim 1, wherein R^{B} is, for each occurrence independently, H or a moiety represented by the following structural formula:

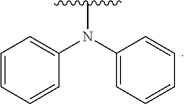 [SMILES: *N(c1ccccc1)c1ccccc1]

11. The molecule of claim 1, wherein R^{B} is, for each occurrence independently, H or a moiety represented by the following structural formula:

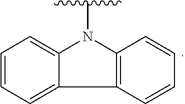 [SMILES: *n1c2ccccc2c2ccccc21]

12. The molecule of claim 1, wherein J is —CN.

13. The molecule of claim 12, wherein the molecule is represented by one of the following structural formulas:

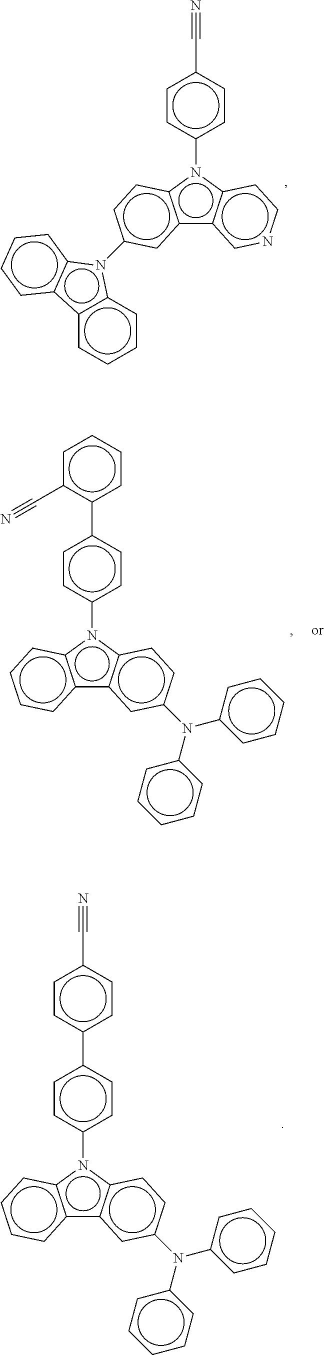

14. The molecule of claim 1, wherein J is

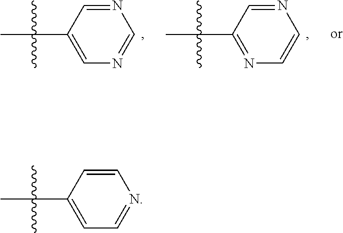 [SMILES: *c1cncnc1 ; *c1cnccn1 ; *c1ccncc1]

15. The molecule of claim 14, wherein the molecule is represented by one of the following structural formulas:

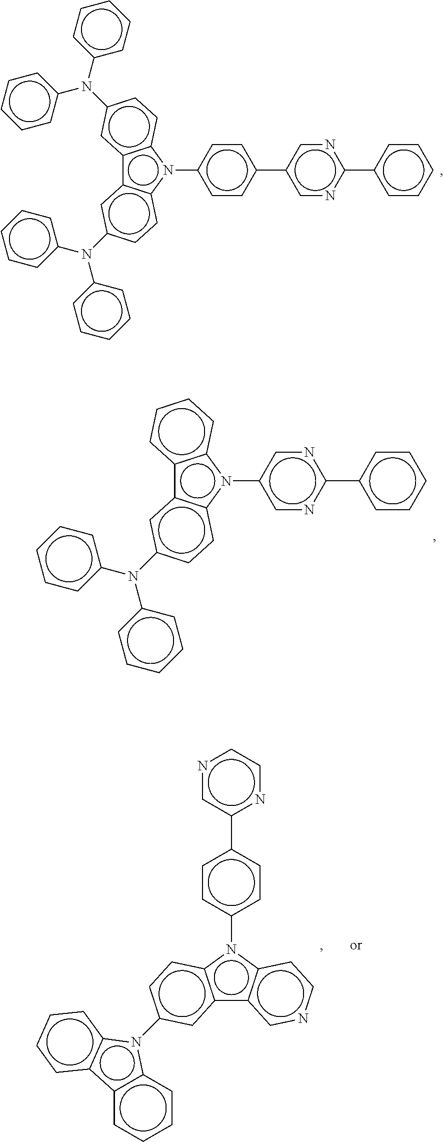

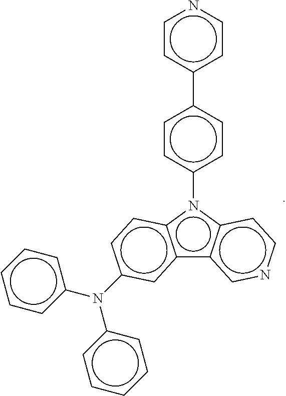

16. The molecule of claim 1, wherein J is

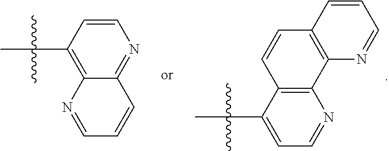 [SMILES: *c1ccnc2cccnc12 ; "or" ; *c1ccnc2c1ccc1cccnc12]

17. The molecule of claim 16, wherein the molecule is represented by one of the following structural formulas:

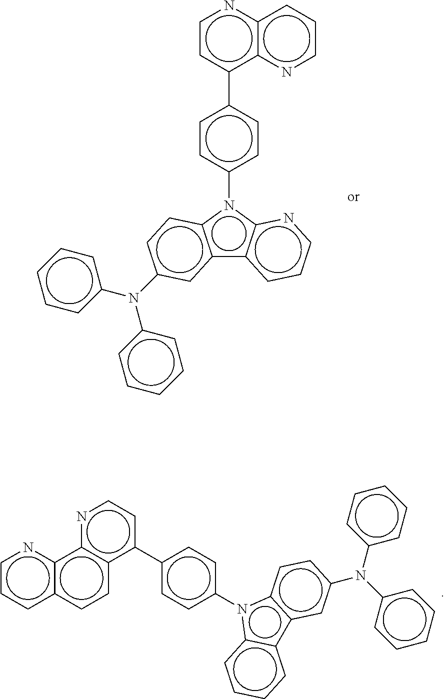

18-127. (canceled)

128. An organic light-emitting device containing:
a first electrode;
a second electrode; and
an organic layer disposed between the first electrode and the second electrode, wherein the organic layer comprises at least one molecule as defined by claim 1.
